(19) United States
(12) Patent Application Publication
(10) Pub. No.: US 2009/0280545 A1
(43) Pub. Date: Nov. 12, 2009

(54) MOLECULE PRODUCTION BY PHOTOSYNTHETIC ORGANISMS

(75) Inventors: Michael Mendez, Del Mar, CA (US); Stephen Mayfield, Cardiff by-the-Sea, CA (US); Bryan O'Neill, San Diego, CA (US); Yan Poon, Pasadena, CA (US); Phillip Lee, San Diego, CA (US); Craig Aaron Behnke, San Diego, CA (US); Su-Chiung Fang, San Diego, CA (US)

Correspondence Address: QUINE INTELLECTUAL PROPERTY LAW GROUP, P.C., P O BOX 458, ALAMEDA, CA, 94501, US

(73) Assignees: Sapphire Energy, San Diego, CA (US); The Scripps Research Institute, La Jolla, CA (US)

(21) Appl. No.: 12/207,761

(22) Filed: Sep. 10, 2008

Related U.S. Application Data
(60) Provisional application No. 60/971,418, filed on Sep. 11, 2007.
(60) Provisional application No. 60/971,412, filed on Sep. 11, 2007.
(60) Provisional application No. 61/130,892, filed on Jun. 2, 2008.

(51) Int. Cl. C12P 7/04; C12N 15/00; C12N 1/13; C12P 5/02

(52) U.S. Cl. 435/157; 435/320.1; 435/317.1; 435/257.2; 435/167

(57) ABSTRACT

The present invention provides compositions and methods for producing products by photosynthetic organisms. The photosynthetic organisms are genetically modified to effect production, secretion, or both, of products. The methods and compositions are particularly useful in the petrochemical industry.

### CROSS-REFERENCE TO RELATED APPLICATIONS

This application claims priority to and benefit of U.S. Provisional Application Nos. 60/971,418 (filed Sep. 11, 2007), 60/971,412 (filed Sep. 11, 2007), and 61/130,892 (filed Jun. 2, 2008), which applications are incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

Fuel products, such as oil, petrochemicals, and other substances useful for the production of petrochemicals are increasingly in demand. Much of today's fuel products are generated from fossil fuels, which are not considered renewable energy sources, as they are the result of organic material being covered by successive layers of sediment over the course of millions of years. There is also a growing desire to lessen dependence on imported crude oil. Public awareness regarding pollution and environmental hazards has also increased. As a result, there has been a growing interest and need for alternative methods to produce fuel products. Thus, there exists a pressing need for alternative methods to develop fuel products that are renewable, sustainable, and less harmful to the environment.

### SUMMARY OF THE INVENTION

The present invention relates to compositions and methods for creating products, such as isoprenoids, which can be used for multiple purposes (e.g., fuel, fuel feedstocks, fragrances and insecticides), using photosynthetic organisms. The compositions include expression vector comprising one or more nucleotide sequences that initiate, increase, or effect the production of a product in a non-vascular, photosynthetic organism.

In some instances, such nucleotide sequence(s) encode one or more polypeptides in the mevalonate pathway (MVA pathway). Examples of polypeptides in the MVA pathway include, but are not limited to, thiolase, HMG-CoA synthase, HMG-CoA reductase, mevalonate kinase, phosphemevalonate kinase, or mevalonate-5-pyrophosphate decarboxylase. In other embodiments, the nucleotide sequence encodes a polypeptide in the non-mevalonate pathway (MEP pathway). The polypeptide may be DOXP synthase, DOXP reductase, 4-diphosphocytidyl-2-C-methyl-D-erythritol synthase, 4-diphophocytidyl-2-C-methyl-D-erythritol kinase, 2-C-methyl-D-erythritol 2,4,-cyclodiphosphate synthase, HMB-PP synthase, HMB-PP reductase, and DOXP reductoisomerase.

One aspect of the present invention provides a vector comprising a nucleic acid encoding an enzyme that produces an isoprenoid with two phosphates and a promoter configured for expression of the nucleic acid in a chloroplast of a non-vascular, photosynthetic organism (NVPO) and does not comprise the entire genome of a chloroplast. In practice, insertion of the vector into a chloroplast genome may not disrupt photosynthetic capability of the chloroplast(s). In other instances, the vectors of the present invention further comprise a nucleic acid sequence which facilitates homologous recombination with a chloroplast genome. In some instances, an isoprenoid produced by an enzyme encoded on a vecor as disclosed herein is GPP, IPP, FPP, GGPP or DMAPP. In some vectors disclosed herein, a second nucleic acid encoding a second enzyme which modifies an isoprenoid with two phosphates is also present on the vector. Specific examples of a second enzyme which may be encoded by the vectors of the present invention include, but are not limited to, botyrococcene synthase, limonene synthase, cineole synthase, pinene synthase, camphene synthase, sabinene synthase, myrcene synthase, abietadiene synthase, taxadiene synthase, FPP synthase, bisabolene synthase, diapophytoene desaturase, diapophytoene synthase, GPP synthase, IPP isomerase, monoterpene synthase, terpinolene synthase, zingiberene synthase, ocimene synthase, sesquiterpene synthase, curcumene synthase, farnesene synthase, geranylgeranyl reductase, chlorophyllidohydrolase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, gamma-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase. The vectors may also contain a selectable marker. Specific vectors of the present invention are capable of stable transformation in microalga. In some embodiments, the algal species is C. reinhardtii, D. salina, H. pluvalis, S. dimorphus, D. viridis, or D. tertiolecta. A nucleic nucleic acid encoding an enzyme may be biased for a nonvascular photosynthetic microorganism nuclear and/or chloroplast expression. Sequences encoding the enzymes useful in the present invention may be any of the sequences specifically disclosed herein (e.g., the sequences in Tables 5-8), or sequences with 60, 65, 70, 75, 80, 85, 90, 95 or higher identity thereto. Some vectors of the present invention comprise any of the sequences specifically disclosed herein (e.g., the sequences in Tables 5-8), or sequences with 60, 65, 70, 75, 80, 85, 90, 95 or higher identity thereto.

Another vector disclosed herein comprises a nucleic acid encoding an enzyme that produces an isoprenoid with two phosphates; and a nucleic acid encoding a chloroplast targeting molecule for targeting the enzyme to a chloroplast. Such vectors may further comprise a selectable marker, a nucleic acid sequence which facilitates homologous recombination with a chloroplast or nuclear genome or a combination of these features. In some instances, the enzyme encoded by the nucleic acid produces GPP, IPP, FPP, GGPP or DMAPP. Such vectors disclosed herein may further comprise a nucleic acid encoding a second enzyme which modifies an isoprenoid with two phosphates and a nucleic acid encoding a chloroplast targeting molecule for targeting the second enzyme to a chloroplast. Specific examples of a second enzyme which may be encoded by the vectors of the present invention include, but are not limited to, botyrococcene synthase, limonene synthase, cineole synthase, pinene synthase, camphene synthase, sabinene synthase, myrcene synthase, abietadiene synthase, taxadiene synthase, FPP synthase, bisabolene synthase, diapophytoene desaturase, diapophytoene synthase, GPP synthase, IPP isomerase, monoterpene synthase, terpinolene synthase, zingiberene synthase, ocimene synthase, sesquiterpene synthase, curcumene synthase, farnesene synthase, geranylgeranyl reductase, chlorophyllidohydrolase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, gamma-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase. Nucleic acid(s) encoding an enzyme for use in the present invention may be codon biased for an NVPO. Specific vectors of the present invention are capable of stable transformation in microalga. In some embodiments, the algal species is C. reinhardtii, D. salina, H. pluvalis, S. dimorphus, D. viridis, or D. tertiolecta. Sequences encoding the enzymes useful in the present invention may be any of the sequences specifically disclosed herein (e.g., the sequences in Table 5-8), or sequences with 60, 65, 70, 75, 80, 85, 90, 95 or higher identity thereto. Some vectors of the present invention comprise any of the sequences specifically disclosed herein (e.g., the sequences in Tables 5-8), or sequences with 60, 65, 70, 75, 80, 85, 90, 95 or higher identity thereto.

The present disclosure also provides a host cell comprising; 1) a vector comprising a nucleic acid encoding an enzyme that produces an isoprenoid with two phosphates and a promoter configured for expression of the nucleic acid in a chloroplast of an NVPO and does not comprise the entire genome of a chloroplast; or 2) a vector comprising a nucleic acid encoding an enzyme that produces an isoprenoid with two phosphates and a nucleic acid encoding a chloroplast targeting molecule for targeting an enzyme to a chloroplast. The host cell may be homoplasmic for one or more of the nucleic acids present on a vector. Some examples of the host cells contemplated herein include cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton. Some specific examples of host cells include the algal species C. reinhardtii, D. salina, H. pluvalis, S. dimorphus, D. viridis, or D. tertiolecta. In some instances, chlorophyll levels are sufficient for the host cell to be photoautotrophic following transformation. In other instances, the host cell may produce at least one naturally occurring isoprenoid at levels greater than a wild-type strain of the same organism.

In another embodiment of the present invention, a host cell is provided which comprises at least two copies of a nucleotide sequence described herein (e.g., in Tables 5-8), or a nucleotide sequence having at least 70% identity to any of these sequences. The host cell may be a non-vascular photosynthetic organism, particularly C. reinhardtii, D. salina, H. pluvalis, S. dimorphus, D. viridis, or D. tertiolecta. In some instances, the host cell is homoplasmic for the nucleotide sequence. The present disclosure also provides a genetically modified chloroplast containing a vector comprising a nucleic acid encoding an enzyme that produces an isoprenoid with two phosphates and a promoter configured for expression of the nucleic acid in a chloroplast of a non-vascular, photosynthetic organism (NVPO).

Also provided herein, is a method for producing an isoprenoid-containing composition comprising the steps of transforming a chloroplast of a non-vascular, photosynthetic organism with a nucleic acid encoding an enzyme that produces an isoprenoid with two phosphates and collecting at least one isoprenoid produced by the transformed NVPO. Such methods may further comprise growing the organism in an aqueous environment, wherein CO₂ is supplied to the organism. CO₂ provided may be at least partially derived from a burned fossil fuel and/or may be at least partially derived from flue gas. Such methods may include production of GPP, IPP, FPP, GGPP or DMAPP. In some instances, the collection process may comprise one or more of the following: harvesting a transformed NVPO, harvesting an isoprenoid from a cell medium; mechanically disrupting a transformed organism and; chemically disrupting an organism. A microalga may be utilized in some aspects of this invention, and the microalga may be C. reinhardtii, D. salina, H. pluvalis, S. dimorphus, D. viridis, or D. tertiolecta.

Another method for producing an isoprenoid is also described wherein the method comprises the steps of transforming the chloroplast of a non-vascular, photosynthetic organism to produce said isoprenoid, wherein said organism is not transformed with isoprene synthase or a methyl-butenol synthase; and (b) collecting said isoprenoid. This method may further comprise growing the organism in an aqueous environment, wherein CO₂ is supplied to the organism. The CO₂ may be at least partially derived from a burned fossil fuel and/or flue gas. Such methods may include production of GPP, IPP, FPP, GGPP or DMAPP. A chloroplast of this method may be transformed with a nucleic acid encoding botyrococcene synthase, limonene synthase, cineole synthase, pinene synthase, camphene synthase, sabinene synthase, myrcene synthase, abietadiene synthase, taxadiene synthase, bisabolene synthase, diapophytoene desaturase, diapophytoene synthase, monoterpene synthase, terpinolene synthase, zingiberene synthase, ocimene synthase, sesquiterpene synthase, curcumene synthase, farnesene synthase, geranylgeranyl reductase, chlorophyllidohydrolase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, gamma-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase. In some instances, the collection process may comprise one or more of the following: harvesting a transformed NVPO, harvesting an isoprenoid from a cell medium; mechanically disrupting a transformed organism and; chemically disrupting an organism. A microalga may be utilized in some aspects of this invention, and the microalga may be C. reinhardtii, D. salina, H. pluvalis, S. dimorphus, D. viridis, or D. tertiolecta.

A further embodiment of the present invention provides an organism with a genetically modified chloroplast wherein the chloroplast comprises a nucleic acid encoding an isoprenoid producing enzyme and wherein the organism can grow in a high saline environment. Such an organism may be an NVPO, specifically D. salina, D. viridis, or D. tertiolecta. In such embodiments, that high saline environment may comprise about 0.5-4.0 molar sodium chloride. Also provided is a method for preparing an isoprenoid comprising transforming an organism with a nucleic acid to increase or initiate production of the isoprenoid, wherein the organism is grown in a high-saline environment; and collecting the isoprenoid. Such an organism may be an NVPO, specifically D. salina, D. viridis, or D. tertiolecta. In such embodiments, that high saline environment may comprise about 0.5-4.0 molar sodium chloride. In some instances, the transforming step is a chloroplast transformation. In other instances, the collecting step comprises one or more of the following steps: (a) harvesting said transformed organism; (b) harvesting said isoprenoid from a cell medium; (c) mechanically disrupting said organism; or (d) chemically disrupting said organism.

The disclosure herein also provides a vector comprising a heterologous nucleic acid encoding one or more isoprenoid producing enzymes; and a promoter configured for expression of said nucleic acid in a photosynthetic bacteria. In some instances, the photosynthetic bacteria is a cyanobacterial species and may be a member of the genera Synechocystis, Synechococcus, and/or Athrospira. A host cell comprising such a vector is provided. A host cell may be a cyanobacterial species and may be a member of the genera Synechocystis, Synechococcus, and/or Athrospira.

The present disclosure further provides a vector comprising: a first nucleic acid encoding a protein, a second nucleic acid encoding a selectable marker, wherein the first and second nucleic acids comprise one open reading frame, and a promoter configured for expression of said first and second nucleic acids in a non-vascular, photosynthetic organism. In some instances, the protein is an isoprenoid producing enzyme or a biomass degrading enzyme. In other instances, the isoprenoid producing enzyme is botyrococcene synthase, limonene synthase, cineole synthase, pinene synthase, camphene synthase, sabinene synthase, myrcene synthase, abietadiene synthase, taxadiene synthase, FPP synthase, bisabolene synthase, diapophytoene desaturase, diapophytoene synthase, GPP synthase, IPP isomerase, monoterpene synthase, terpinolene synthase, zingiberene synthase, ocimene synthase, sesquiterpene synthase, curcumene synthase, farnesene synthase, geranylgeranyl reductase, chlorophyllidohydrolase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, gamma-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase. In still other instances, the biomass degrading enzyme is exo-β-glucanase, endo-β-glucanase, β-glucosidase, endoxylanase, or ligninase. Some of the vectors described herein comprise a first or second nucleic acid in which at least one codon is optimized for expression in the nucleus of a non-vascular, photosynthetic organism is present.

In some instances, vectors described herein comprise a third nucleic acid encoding a cleavage moiety in-frame with said first and second nucleic acids. The cleavage moiety may be a self-cleaving protease and may specifically be a functional portion of the A2 region from foot and mouth disease virus. In other instances, the cleavage moiety is capable of being cleaved by a protease naturally produced by said organism. Also described herein are vectors comprising regulatory elements including an HSP70 promoter, a functional portion of HSP70 promoter, rbcS2 5′ upstream translated region (UTR), a functional portion of rbcS2 5′ UTR, or a combination thereof. In some instances, the regulatory element is derived from the organism to be transformed. Still other vectors comprise a fourth nucleic acid encoding a secretion signal in-frame with the first, second and/or third nucleic acids. A secretion signal useful in the present vectors is a C. reinhardtii carbonic anhydrase secretion signal. Vectors of the invention may be useful in multiple NVPOs, including photosynthetic bacteria, cyanobacteria, cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton. In some instances, the vector is capable of stable transformation in C. reinhardtii, D. salina, H. pluvalis, S. dimorphus, D. viridis, D. tertiolecta, a bacterium of the genus Synechocystis, a bacterium of the genus Synechococcus, or a bacterium of the genus Athrospira. The vectors described herein may comprise any nucleotide sequence described herein (e.g., in Tables 5-8), or a nucleotide sequence having at least 70% identity to any of these sequences. Still other vectors comprise a fifth nucleic acid in-frame with said first, second, third and/or fourth nucleic acids, where the fifth nucleic acid encodes a tag. The encoded tag may be an epitope tag or a metal affinity tag.

The present disclosure also provides a host cell comprising a vector, wherein the vector comprises a first nucleic acid encoding a protein, a second nucleic acid encoding a selectable marker, wherein the first and second nucleic acids comprise one open reading frame, a promoter configured for expression of the first and second nucleic acids in a non-vascular, photosynthetic organism and optionally one or more additional nucleic acids encoding a cleavage moiety, a secretion signal, a tag or a combination thereof, wherein the one or more additional nucleic acids are in-frame with said first and second nucleic acids. The host cell may be a photosynthetic bacteria, cyanobacteria, cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, or phytoplankton. In some instances, the host cell is C. reinhardtii, D. salina, H. pluvalis, S. dimorphus, D. viridis, D. tertiolecta, a bacterium of the genus Synechocystis, a bacterium of the genus Synechococcus, or a bacterium of the genus Athrospira. In other instances, the vector is stably integrated into the nuclear genome of said host cell.

Further provided herein is a method of producing a protein in a non-vascular photosynthetic organism, comprising: growing said organism, wherein the organism comprises an exogenous nucleic acid comprising a single open reading frame, wherein the open reading frame comprises a first nucleic acid encoding a protein and a second nucleic acid encoding a selectable marker, and wherein the organism further comprises a promoter configured for expression of said open reading frame in said organism, thereby producing said protein. In some instances, the protein is an isoprenoid producing enzyme or a biomass degrading enzyme. In other instances, the open reading frame comprises at least one codon optimized for expression in the nucleus of a non-vascular, photosynthetic organism. An open reading frame may further comprise a third nucleic acid encoding a cleavage moiety in-frame with the first and second nucleic acids. The cleavage moiety may be a self-cleaving protease, and in a particular embodiment may be a functional portion of the A2 region from foot and mouth disease virus. In other embodiments, a cleavage moiety is capable of being cleaved by a protease naturally produced by said organism. An open reading frame may further comprise a fourth nucleic acid encoding a secretion signal in-frame with all other nucleic acids comprising the open reading frame. In one embodiment, the secretion signal is a C. reinhardtii carbonic anhydrase secretion signal. As disclosed herein, the organism useful for such a method may be C. reinhardtii, D. salina, H. pluvalis, S. dimorphus, D. viridis, D. tertiolecta, a bacterium of the genus Synechocystis, the genus Synechococcus, or the genus Athrospira. An open reading frame for use in this method may further comprise a fifth nucleic acid encoding a tag in-frame with all other nucleic acids comprising the open reading frame. The tag may be an epitope tag or a metal affinity tag.

Further disclosed herein is a host cell comprising a fusion protein, wherein the fusion protein comprises a first nucleic acid encoding a protein and a second nucleic acid encoding a selectable marker and wherein the host cell is a non-vascular photosynthetic organism. The host cell may be C. reinhardtii, D. salina, H. pluvalis, a bacterium of the genus Synechocystis, the genus Synechococcus, or the genus Athrospira. In some instances, the vector is stably integrated into a nuclear genome of the host cell. A fusion protein may further comprise a cleavage moiety, a secretion signal, a tag, or a combination thereof. A cleavage moiety may be a self-cleaving protease, such as a functional portion of the A2 region from foot and mouth disease virus. Alternately, a cleavage moiety may capable of being cleaved by a protease naturally produced by said organism. One secretion signal which may be utilized is a C. reinhardtii carbonic anhydrase secretion signal. In fusion proteins comprising a tag, the tag may be an epitope tag or a metal affinity tag.

Provided herein is a method of producing a transgenic non-vascular photosynthetic organism expressing a protein of interest under selective conditions, where the method comprises the step of: transforming the organism with a nucleic acid comprising a single open reading frame, wherein the open reading frame encodes a fusion protein comprising said protein of interest and a selectable marker; wherein the organism is capable of expressing the selectable marker under environmental conditions which require expression of the selectable marker for continued viability of the organism, thereby resulting in expression of said protein of interest. In some instances, the protein is an isoprenoid producing enzyme or a biomass degrading enzyme. A fusion protein may further comprise a cleavage moiety, a secretion signal, a tag, or a combination thereof. A cleavage moiety may be a self-cleaving protease, such as a functional portion of the A2 region from foot and mouth disease virus. Alternately, a cleavage moiety may capable of being cleaved by a protease naturally produced by said organism. One secretion signal which may be utilized is a C. reinhardtii carbonic anhydrase secretion signal. In fusion proteins comprising a tag, the tag may be an epitope tag or a metal affinity tag.

Further provided by the disclosure herein is a method of increasing phytol production in a non-vascular photosynthetic organism, comprising the step of transforming the organism with a nucleic acid which results in an increase in production of phytol by the organism above a level produced by the organism not containing said nucleic acid. In some instances, the nucleic acid encodes a GPP synthase, a FPP synthase, a geranylgeranyl reductase, a chlorophyllidohydrolase, or a pyrophosphatase. In some embodiments, a transformation step may comprise a chloroplast transformation. In still other embodiments, the enzyme is endogenous to the organism or is homologous to an endogenous enzyme of the organism or is exogenous to the organism. In some instances, the enzyme is overexpressed. Expression of the enzyme may be regulated by an inducible promoter. The disclosed method may further comprise transformation of the organism with a nucleic acid which results in production of dimethylallyl alcohol, isopentyl alcohol, geraniol, farnesol or geranylgeraniol. In some instances, the organism used in practicing the method is a photosynthetic bacteria, cyanobacteria, cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton. The organism may be C. reinhardtii, D. salina, H. pluvalis, S. dimorphus, D. viridis, or D. tertiolecta.

Also provided herein, is a host cell comprising an introduced nucleic acid, wherein the nucleic acid results in an increase in production of phytol by the host cell above a level produced by a host cell not containing the nucleic acid, wherein the host cell is a non-vascular photosynthetic organism. In some embodiments, the host cell can grow in a high-saline environment, for example, the host cell may be, D. viridis, or D. tertiolecta. In some instances, the high-saline environment comprises 0.5-4.0 molar sodium chloride. In some host cells, the nucleic acid is present in a chloroplast. In still other host cells, the nucleic acid encodes an enzyme selected from the group consisting of a GPP synthase, a FPP synthase, a geranylgeranyl reductase, a chlorophyllidohydrolase, and a pyrophosphatase. The host cell may further comprise a nucleic acid which results in production of dimethylallyl alcohol, isopentyl alcohol, geraniol, farnesol or geranylgeraniol. The host cell may be a photosynthetic bacteria, cyanobacteria, cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton. In some instances, the host cell is C. reinhardtii, D. salina, H. pluvalis, S. dimorphus, D. viridis, or D. tertiolecta.

Another method disclosed herein provides a method of producing phytol in a non-vascular photosynthetic organism, comprising the steps of transforming the organism with a nucleic acid which results in an increase in production of phytol by the organism above a level produced under given environmental conditions; and collecting the phytol from the organism. In some instances of this method, the nucleic acid encodes a GPP synthase, a FPP synthase, a geranylgeranyl reductase, a chlorophyllidohydrolase, or a pyrophosphatase. In still other instances, the transformation is a chloroplast transformation. In still other embodiments, the enzyme is endogenous to the organism or is homologous to an endogenous enzyme of the organism or is exogenous to the organism. In some instances, the enzyme is overexpressed. Expression of the enzyme may be regulated by an inducible promoter. The disclosed method may further comprise transformation of the organism with a nucleic acid which results in production of dimethylallyl alcohol, isopentyl alcohol, geraniol, farnesol or geranylgeraniol. In some instances, the organism used in practicing the method is a photosynthetic bacteria, cyanobacteria, cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton. The organism may be C. reinhardtii, D. salina, H. pluvalis, S. dimorphus, D. viridis, or D. tertiolecta.

Further provided herein is a composition comprising at least 3% phytol and at least a trace amount of a cellular portion of a genetically modified non-vascular photosynthetic organism. In some instances, the genetically modified organism is modified by an endogenous, heterologous, or exogenous GPP synthase, FPP synthase, geranylgeranyl reductase, chlorophyllidohydrolase, or pyrophosphatase. In other instances, a chloroplast of the organism is genetically modified. The disclosed compositions may further comprise dimethylallyl alcohol, isopentyl alcohol, geraniol, farnesol or geranylgeraniol. In some instances, the cellular portion present in the composition is a from a photosynthetic bacteria, cyanobacteria, cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton. In other instances, the organism may be C. reinhardtii, D. salina, H. pluvalis, S. dimorphus, D. viridis, or D. tertiolecta.

In some instances, such nucleotide sequence(s) encode one or more polypeptides that function in isoprenoid synthetic pathway. Examples of polypeptides in the isoprenoid biosynthetic pathway include synthases such as C5, C10, C15, C20, C30, and C40 synthases. More specific examples of polypeptides in the isoprenoid pathway limonene synthase, 1,8 cineole synthase, α-pinene synthase, camphene synthase, (+)-sabinene synthase, myrcene synthase, abietadiene synthase, taxadiene synthase, farnesyl pyrophosphate synthase, amorphadiene synthase, (E)-α-bisabolene synthase, diapophytoene synthase, or diapophytoene desaturase. In other embodiments, the synthase is β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, δ-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase.

Any of the nucleotides sequences contemplated herein can include one or more heterologous sequences and/or one or more homologous sequences.

In some instances, the products produced can be naturally produced by the organism that is transformed. In other instances, the products are not naturally produced by the organism that is transformed.

In some instances, a product (e.g. fuel, fuel feedstock, fragrance, insecticide) is a hydrocarbon-rich molecule, e.g. an isoprenoid. An isoprenoid (classified by the number of isoprene units) can be a hemiterpene, monoterpene, sesquiterpene, diterpene, triterpene, or tetraterpene. In specific embodiments, the isoprenoid may be a naturally occurring isoprenoid, such as a steroid or carotenoid. Subclasses of carotenoids include carotenes and xanthophylls. Some isoprenoids are pure hydrocarbons (e.g. limonene) and others are hydrocarbon derivatives (e.g. cineole).

Any of the nucleotide sequences herein can further include codons biased for expression of the nucleotide sequences in the organism transformed. In some instances, codons in the nucleotide sequences are A/T rich in a third nucleotide position of the codons. For example, at least 50% of the third nucleotide position of the codons may be A or T. In other instances, the codons are G/C rich, for example at least 50% of the third nucleotide positions of the codons may be G or C.

The nucleotide sequences herein can be adapted for chloroplast expression. For example, a nucleotide sequence herein can comprise a chloroplast specific promoter or chloroplast specific regulatory control region. The nucleotide sequences can also be adapted for nuclear expression. For example, a nucleotide sequence can comprise a nuclear specific promoter or nuclear specific regulatory control regions. The nuclear sequences can encode a protein with a targeting sequence that encodes a chloroplast targeting protein (e.g., a chloroplast transit peptide), or a signal peptide that directs a protein to the endomembrane system for deposition in the endoplasmic reticulum or plasma membrane.

Fuel products are produced by altering the enzymatic content of the cell to increase the biosynthesis of specific fuel molecules. For example, nucleotide sequences encoding biosynthetic enzymes can be introduced into the chloroplast of a photosynthetic organism. Nucleotide sequences encoding fuel biosynthetic enzymes can also be introduced into the nuclear genome of the photosynthetic organisms. Nucleotide sequences introduced into the nuclear genome can direct accumulation of the biosynthetic enzyme in the cytoplasm of the cell, or may direct accumulation of the biosynthetic enzyme in the chloroplast of the photosynthetic organism.

Any of the nucleotide sequences herein may further comprise a regulatory control sequence. Regulatory control sequences can include one or more of the following: a promoter, an intron, an exon, processing elements, 3′ untranslated region, 5′ untranslated region, RNA stability elements, or translational enhancers A promoter may be one or more of the following: a promoter adapted for expression in the organism, an algal promoter, a chloroplast promoter, and a nuclear promoter, any of which may be a native or synthetic promoters. A regulatory control sequence can be inducible or autoregulatable. A regulatory control sequence can include autologous and/or heterologous sequences. In some cases, control sequences can be flanked by a first homologous sequence and a second homologous sequence. The first and second homologous sequences can each be at least 500 nucleotides in length. The homologous sequences can allow for either homologous recombination or can act to insulate the heterologous sequence to facilitate gene expression.

In some instances, a nucleotide sequence may allow for secretion of the product (e.g., a protein) from the cell. In these cases, the nucleotide sequences herein may encode a protein that enhances or initiates or increases the rate of secretion of a product from an organism to the external environment.

The present invention also contemplates organisms transformed with the one or more nucleotide sequences or expression vectors herein. Such organisms are preferably photosynthetic and can be, e.g., unicellular or mutlicellular. For example, such organisms can be multicellular or unicellular algae or cyanobacteria. Some examples of algae contemplated herein include rhodophyta, chlorophyta, heterokontophyta, tribophyta, glaucophyta, chlorarachniophytes, euglenoids, haptophyta, cryptomonads, dinoflagellata, and phytoplankton.

Any of the organisms contemplated herein can be transiently or stably transformed with one or more of the expression vectors described herein. Preferably, the production of the product by the organism does not render the organism unviable.

The present invention also contemplates methods for producing a fuel product. The method can include transforming a non-vascular, photosynthetic organism with an expression vector, growing the organism; and collecting the fuel product produced by the organism. The expression vector can encode a protein that alters the biosynthetic pathway of a photosynthetic organism to allow for increased production or accumulation of a fuel molecule. The expression vector might also encode regulatory elements that alter a native enzyme in a biosynthetic pathway to allow for increased fuel production or accumulation. The vector may also encode a protein or regulatory elements that allows for secretion or increased secretion of a fuel molecule.

The present invention also provides a business method comprising providing a carbon credit to a party growing a genetically modified non-vascular, photosynthetic organism adapted to produce a fuel product. The organism may be any of the ones described herein. In some embodiments, the carbon credit is exchanged for one or more of the following: a substantially liquid monetary instrument, commitment of at least one of present and future business opportunity, a legal grant regarding an intellectual property right, government tax subsidy, access to purchasers of a given market; or use of a carbon emission process not comprising growing the organism. The carbon credit may be substantially received directly from a regulatory agency. Alternatively, the carbon credit is substantially received directly from an administrative entity. The carbon credit may be regulated by at least one entity selected from the group consisting of: a city, county, state, provincial, national, regional, multi-national, and international sovereign entity.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a representation of a naturally occurring enzyme pathway in C. reinhardtii.

FIG. 2 is a representation of one example of a modification of enzyme pathway in C. reinhardtii.

FIG. 3, panels A-D provide a schematic representation of nucleic acid constructs of the present invention.

FIG. 4, panels A-D show PCR and Western analysis of C. reinhardtii transformed with FPP synthase and bisabolene synthase.

FIG. 5 shows gas chromatography—mass spectrometry analysis of C. reinhardtii transformed with FPP synthase and bisabolene synthase.

FIG. 6, panels A-E show PCR and Western analysis of C. reinhardtii transformed with FPP synthase and squalene synthase.

FIG. 7 shows gas chromatography—mass spectrometry analysis of C. reinhardtii transformed with FPP synthase and squalene synthase.

FIG. 8 shows Western analysis of C. reinhardtii transformed with limonene synthase.

FIG. 9 shows gas chromatography—mass spectrometry analysis of C. reinhardtii transformed with limonene synthase.

FIG. 10, panels A-C show PCR and Western analysis of C. reinhardtii transformed with GPP synthase.

FIG. 11 shows PCR and Western analysis of C. reinhardtii transformed with FPP synthase and zingiberene synthase.

FIG. 12 shows Western analysis of C. reinhardtii transformed with FPP synthase and sesquiterpene synthase.

FIG. 13 shows gas chromatography—mass spectrometry analysis of phytol production in C. reinhardtii transformed with FPP synthase and sesquiterpene synthase.

FIG. 14 shows Western analysis of E. coli transformed with FPP synthase and sesquiterpene synthase.

FIG. 15 shows gas chromatography—mass spectrometry analysis of FPP and sesquiterpene production in E. coli transformed with FPP synthase and sesquiterpene synthase.

FIG. 16 is a graphic representation of nucleic acid constructs of the present invention.

FIG. 17 shows Western analysis of C. reinhardtii expressing xylanase 2 from the nucleus.

FIG. 18 shows a comparison of xylanase activity from exogenous enzymes expressed in the nucleus and chloroplast of C. reinhardtii.

FIG. 19 shows Western analysis of C. reinhardtii expressing endoglucanase from the nucleus.

FIG. 20 shows Western analysis of C. reinhardtii expressing CBH1 from the nucleus.

### DETAILED DESCRIPTION OF THE INVENTION

The present invention relates to compositions and methods for creating product(s) using one or more photosynthetic organisms. In some instances, the photosynthetic organisms are non-vascular organisms (e.g., cyanobacteria, algae). As detailed herein, a non-vascular photosynthetic organism (NVPO) may be transformed with exogenous, heterologous or autologous nucleic acids which encode one or more enzymes that effect the production of the product(s) of the invention. For example, an NVPO (e.g., C. reinhardtii) may be transformed with one or more nucleic acids encoding enzyme(s) (e.g., bisabolene synthase, sesquiterpene synthase) which effect the production of a desired product (e.g., bisabolene, squalene). The product(s) produced may be naturally, or not naturally, produced by the photosynthetic organism. When naturally produced, production may be enhanced by introduction of the nucleic acids of the present invention. For example, transformation of an NVPO with one or more nucleic acids encoding enzymes which effect the production of a desired product (e.g., zingiberene, bisabolene), may result in increased production of another product (e.g., phytol). In still other instances, multiple products may be produced by a transformed NVPO and the multiple products may be naturally occurring, non-naturally occurring, or a combination thereof. The compositions of the present invention may comprise mixtures of naturally and non-naturally occurring products in a ratio of 1:0.1, 1:0.2, 1:0.3, 1:0.4, 1:0.5, 1:0.6, 1:0.7, 1:0.8, 1:0.9, 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100 or higher.

The products which are produced by the methods of the present invention include hydrocarbons and hydrocarbon derivatives. In certain aspects, the hydrocarbon and/or derivative is an isoprenoid (or terpenoid). The isoprenoids contemplated by the present invention may contain any number of carbon atoms, with isoprenoids containing five to fifty carbon atoms being exemplary. An isoprenoid of the present invention may be one naturally produced by the NVPO prior to transformation (e.g., phytol), or may be produced only after insertion of an exogenous nucleic acid (e.g., zingiberene). A product of the present invention may also contain one or more non-naturally produced isoprenoids in addition to one or more naturally produced isoprenoids. Additionally, the products are produced intracellularly and may be sequestered in the organism. Thus, collection of the product may involve disruption of one or more cells of the organism(s) of the present invention and/or collection of the product from the environment surrounding the organism(s). Collection of the product(s) of the present invention may involve collecting all or part of a liquid environment in which the cells are grown, isolating the cells from the liquid environment, and disrupting the cells prior to or following isolation from the growth environment, or a combination of these.

The collected product may be purified (e.g., refined) following collection. The product may be utilized in the form in which it is collected, or may be altered prior to, or after collection. For example, where the product is a sesquiterpene (C15), the sesquiterpene may be hydrogenated, cracked, or otherwise modified, resulting in a compound with a different number of carbon atoms. In some instances, alteration of the product may yield a fuel product (e.g., octane, butane).

Organisms

Examples of organisms that can be transformed using the compositions and methods herein include vascular and non-vascular organisms. The organism can be prokaroytic or eukaroytic. The organism can be unicellular or multicellular.

Examples of non-vascular photosynthetic organisms include bryophtyes, such as marchantiophytes or anthocerotophytes. In some instances, the organism is a cyanobacteria. In some instances, the organism is algae (e.g., macroalgae or microalgae). The algae can be unicellular or multicellular algae. In some instances, the organism is a rhodophyte, chlorophyte, heterokontophyte, tribophyte, glaucophyte, chlorarachniophyte, euglenoid, haptophyte, cryptomonad, dinoflagellum, or phytoplankton. For example, the microalgae Chlamydomonas reinhardtii may be transformed with a vector, or a linearized portion thereof, encoding limonene synthase to produce limonene.

The methods of the present invention are exemplified using the microalga, C. reinhardtii. The use of microalgae to express a polypeptide or protein complex according to a method of the invention provides the advantage that large populations of the microalgae can be grown, including commercially (Cyanotech Corp.; Kailua-Kona Hi.), thus allowing for production and, if desired, isolation of large amounts of a desired product. However, the ability to express, for example, functional mammalian polypeptides, including protein complexes, in the chloroplasts of any plant allows for production of crops of such plants and, therefore, the ability to conveniently produce large amounts of the polypeptides. Accordingly, the methods of the invention can be practiced using any plant having chloroplasts, including, for example, microalga and macroalgae, for example, marine algae and seaweeds, as well as plants that grow in soil.

The term “plant” is used broadly herein to refer to a eukaryotic organism containing plastids, particularly chloroplasts, and includes any such organism at any stage of development, or to part of a plant, including a plant cutting, a plant cell, a plant cell culture, a plant organ, a plant seed, or a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like.

A method of the invention can generate a plant containing chloroplasts that are genetically modified to contain a stably integrated polynucleotide (Hager and Bock, Appl. Microbiol. Biotechnol. 54:302-310, 2000). Accordingly, the present invention further provides a transgenic (transplastomic) plant, e.g. C. reinhardtii, which comprises one or more chloroplasts containing a polynucleotide encoding one or more heterologous polypeptides, including polypeptides that can specifically associate to form a functional protein complex. A photosynthetic organism of the present invention comprises at least one host cell that is modified to generate a product.

Vectors, Transformation and Methods.

The organisms/host cells herein can be transformed to modify the production and/or secretion of a product(s) with an expression vector, or a linearized portion thereof, for example, to increase production and/or secretion of a product(s). The product(s) can be naturally or not naturally produced by the organism.

The expression vector, or a linearized portion thereof, can encode one or more homologous or heterologous nucleotide sequences (derived from the host organism or from a different organism) and/or one or more autologous nucleotide sequences (derived from the same organism) and/or those that encode homologous or heterologous polypeptides. Examples of heterologous nucleotide sequences that can be transformed into an algal host cell include genes from bacteria, fungi, plants, photosynthetic bacteria or other algae. Examples of autologous nucleotide sequences that can be transformed into an algal host cell include isoprenoid producing genes, including genes which encode for proteins which produce isoprenoids with two phosphates (e.g., GPP synthase, FPP synthase), endogenous promoters and 5′ UTRs from the psbA, atpA, or rbcL genes. In some instances, a heterolgous sequence is flanked by two autologous sequences or homologous sequences. Homologous sequences include those that have at least 50%, 60%, 70%, 80%, or 90% homology to the sequence in the host cell. In some instances, a homologous sequence is flanked by two autologous sequences. The first and second homologous sequences enable recombination of the heterologous sequence into the genome of the host organism. The first and second homologous sequences can be at least 100, 200, 300, 400, or 500 nucleotides in length.

The expression vector may comprise nucleotide sequences that are codon biased for expression in the organism being transformed. The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. In some organisms, codon bias differs between the nuclear genome and organelle genomes, thus, codon optimization or biasing may be performed for the target genome (e.g., nuclear codon biased, chloroplast codon biased). The codons of the present invention are generally A/T rich, for example, A/T rich in the third nucleotide position of the codons. Typically, the A/T rich codon bias is used for algae. In some embodiments, at least 50% of the third nucleotide position of the codons are A or T. In other embodiments, at least 60%, 70%, 80%, 90%, or 99% of the third nucleotide position of the codons are A or T.

One approach to construction of a genetically manipulated strain of alga involves transformation with a nucleic acid which encodes a gene of interest, typically an enzyme capable of converting a precursor into a fuel product or precursor of a fuel product. In some embodiments, a transformation may introduce nucleic acids into any plastid of the host alga cell (e.g., chloroplast). Transformed cells are typically plated on selective media following introduction of exogenous nucleic acids. This method may also comprise several steps for screening. Initially, a screen of primary transformants is typically conducted to determine which clones have proper insertion of the exogenous nucleic acids. Clones which show the proper integration may be propagated and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (e.g., nested PCR, real time PCR). For any given screen, one of skill in the art will recognize that PCR components may be varied to achieve optimal screening results. For example, magnesium concentration may need to be adjusted upwards when PCR is performed on disrupted alga cells to which EDTA (which chelates magnesium) is added to chelate toxic metals. In such instances, magnesium concentration may need to be adjusted upward, or downward (compared to the standard concentration in commercially available PCR kits) by 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mM. Thus, after adjusting, final magnesium concentration in a PCR reaction may be, for example 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 mM or higher. Particular examples are utilized in the examples described herein; however, one of skill in the art will recognize that other PCR techniques may be substituted for the particular protocols described. Following screening for clones with proper integration of exogenous nucleic acids, typically clones are screened for the presence of the encoded protein. Protein expression screening typically is performed by Western blot analysis and/or enzyme activity assays.

A recombinant nucleic acid molecule useful in a method of the invention can be contained in a vector. Furthermore, where the method is performed using a second (or more) recombinant nucleic acid molecules, the second recombinant nucleic acid molecule also can be contained in a vector, which can, but need not, be the same vector as that containing the first recombinant nucleic acid molecule. The vector can be any vector useful for introducing a polynucleotide into a chloroplast and, preferably, includes a nucleotide sequence of chloroplast genomic DNA that is sufficient to undergo homologous recombination with chloroplast genomic DNA, for example, a nucleotide sequence comprising about 400 to 1500 or more substantially contiguous nucleotides of chloroplast genomic DNA. Chloroplast vectors and methods for selecting regions of a chloroplast genome for use as a vector are well known (see, for example, Bock, J. Mol. Biol. 312:425-438, 2001; see, also, Staub and Maliga, Plant Cell 4:39-45, 1992; Kavanagh et al., Genetics 152:1111-1122, 1999, each of which is incorporated herein by reference).

In some instances, such vectors include promoters. Promoters useful for the present invention may come from any source (e.g., viral, bacterial, fungal, protist, animal). The promoters contemplated herein can be specific to photosynthetic organisms, non-vascular photosynthetic organisms, and vascular photosynthetic organisms (e.g., algae, flowering plants). As used herein, the term “non-vascular photosynthetic organism,” refers to any macroscopic or microscopic organism, including, but not limited to, algae, cyanobacteria and photosynthetic bacteria, which does not have a vascular system such as that found in higher plants. In some instances, the nucleic acids above are inserted into a vector that comprises a promoter of a photosynthetic organism, e.g., algae. The promoter can be a promoter for expression in a chloroplast and/or other plastid. In some instances, the nucleic acids are chloroplast based. Examples of promoters contemplated for insertion of any of the nucleic acids herein into the chloroplast include those disclosed in US Application No. 2004/0014174. The promoter can be a constitutive promoter or an inducible promoter. A promoter typically includes necessary nucleic acid sequences near the start site of transcription, (e.g., a TATA element).

A “constitutive” promoter is a promoter that is active under most environmental and developmental conditions. An “inducible” promoter is a promoter that is active under environmental or developmental regulation. Examples of inducible promoters/regulatory elements include, for example, a nitrate-inducible promoter (Bock et al, Plant Mol. Biol. 17:9 (1991)), or a light-inducible promoter, (Feinbaum et al, Mol. Gen. Genet. 226:449 (1991); Lam and Chua, Science 248:471 (1990)), or a heat responsive promoter (Muller et al., Gene 111: 165-73 (1992)).

The entire chloroplast genome of C. reinhardtii is available to the public on the world wide web, at the URL “biology.duke.edu/chlamy_genome/-chloro.html” (see “view complete genome as text file” link and “maps of the chloroplast genome” link), each of which is incorporated herein by reference (J. Maul, J. W. Lilly, and D. B. Stern, unpublished results; revised Jan. 28, 2002; to be published as GenBank Acc. No. AF396929). Generally, the nucleotide sequence of the chloroplast genomic DNA is selected such that it is not a portion of a gene, including a regulatory sequence or coding sequence, particularly a gene that, if disrupted due to the homologous recombination event, would produce a deleterious effect with respect to the chloroplast, for example, for replication of the chloroplast genome, or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector of the invention. For example, the chloroplast vector, p322, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link; also accessible directly on world wide web at URL “biology.duke.edu/chlam-y/chloro/chloro140.html”).

A vector utilized in the practice of the invention also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, sequences that encode a selectable marker, and the like. As such, the vector can contain, for example, one or more cloning sites such as a multiple cloning site, which can, but need not, be positioned such that a heterologous polynucleotide can be inserted into the vector and operatively linked to a desired element. The vector also can contain a prokaryote origin of replication (ori), for example, an E. coli ori or a cosmid ori, thus allowing passage of the vector in a prokaryote host cell, as well as in a plant chloroplast, as desired.

A regulatory element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, and an IRES. Additionally, a cell compartmentalization signal (i.e., a sequence that targets a polypeptide to the cytosol, nucleus, chloroplast membrane or cell membrane). In some aspects of the present invention, a cell compartmentalization signal (e.g., a chloroplast targeting sequence) may be ligated to a gene and/or transcript, such that translation of the gene occurs in the chloroplast. In other aspects, a cell compartmentalization signal may be ligated to a gene such that, following translation of the gene, the protein is transported to the chloroplast. Such signals are well known in the art and have been widely reported. See, e.g., U.S. Pat. No. 5,776,689; Quinn et al., J. Biol. Chem. 1999; 274(20): 14444-54; von Heijne et al., Eur. J. Biochem. 1989; 180(3): 535-45.

A vector, or a linearized portion thereof, may include a nucleotide sequence encoding a reporter polypeptide or other selectable marker. The term “reporter” or “selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (Giacomin, Plant Sci. 116:59-72, 1996; Scikantha, J. Bacteriol. 178:121, 1996; Gerdes, FEBS Lett. 389:44-47, 1996; see, also, Jefferson, EMBO J. 6:3901-3907, 1997, f1-glucuronidase). A selectable marker generally is a molecule that, when present or expressed in a cell, provides a selective advantage (or disadvantage) to the cell containing the marker, for example, the ability to grow in the presence of an agent that otherwise would kill the cell.

A selectable marker can provide a means to obtain prokaryotic cells or plant cells or both that express the marker and, therefore, can be useful as a component of a vector of the invention (see, for example, Bock, supra, 2001). One class of selectable markers are native or modified genes which restore a biological or physiological function to a host cell (e.g., restores photosynthetic capability, restores a metabolic pathway). Other examples of selectable markers include, but are not limited to, those that confer antimetabolite resistance, for example, dihydrofolate reductase, which confers resistance to methotrexate (Reiss, Plant Physiol. (Life Sci. Adv.) 13:143-149, 1994); neomycin phosphotransferase, which confers resistance to the aminoglycosides neomycin, kanamycin and paromycin (Herrera-Estrella, EMBO J. 2:987-995, 1983), hygro, which confers resistance to hygromycin (Marsh, Gene 32:481-485, 1984), trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (White et al., Nucl. Acids Res. 18:1062, 1990; Spencer et al., Theor. Appl. Genet. 79:625-631, 1990), a mutant EPSPV-synthase, which confers glyphosate resistance (Hinchee et al., BioTechnology 91:915-922, 1998), a mutant acetolactate synthase, which confers imidazolione or sulfonylurea resistance (Lee et al., EMBO J. 7:1241-1248, 1988), a mutant psbA, which confers resistance to atrazine (Smeda et al., Plant Physiol. 103:911-917, 1993), or a mutant protoporphyrinogen oxidase (see U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells and tetracycline; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide and sulfonylurea resistance in plants (see, for example, Maliga et al., Methods in Plant Molecular Biology, Cold Spring Harbor Laboratory Press, 1995, page 39).

Reporter genes have been successfully used in chloroplasts of higher plants, and high levels of recombinant protein expression have been reported. In addition, reporter genes have been used in the chloroplast of C. reinhardtii, but, in most cases very low amounts of protein were produced. Reporter genes greatly enhance the ability to monitor gene expression in a number of biological organisms. In chloroplasts of higher plants, ββ-glucuronidase (uidA, Staub and Maliga, EMBO J. 12:601-606, 1993), neomycin phosphotransferase (nptII, Carrer et al., Mol. Gen. Genet. 241:49-56, 1993), adenosyl-3-adenyltransf-erase (aadA, Svab and Maliga, Proc. Natl. Acad. Sci., USA 90:913-917, 1993), and the Aequorea victoria GFP (Sidorov et al., Plant J. 19:209-216, 1999) have been used as reporter genes (Heifetz, Biochemie 82:655-666, 2000). Each of these genes has attributes that make them useful reporters of chloroplast gene expression, such as ease of analysis, sensitivity, or the ability to examine expression in situ. Based upon these studies, other heterologous proteins have been expressed in the chloroplasts of higher plants such as Bacillus thuringiensis Cry toxins, conferring resistance to insect herbivores (Kota et al., Proc. Natl. Acad. Sci., USA 96:1840-1845, 1999), or human somatotropin (Staub et al., Nat. Biotechnol. 18:333-338, 2000), a potential biopharmaceutical. Several reporter genes have been expressed in the chloroplast of the eukaryotic green alga, C. reinhardtii, including aadA (Goldschmidt-Clermont, Nucl. Acids Res. 19:4083-4089 1991; Zerges and Rochaix, Mol. Cell. Biol. 14:5268-5277, 1994), uidA (Sakamoto et al., Proc. Natl. Acad. Sci., USA 90:477-501, 19933, Ishikura et al., J. Biosci. Bioeng. 87:307-314 1999), Renilla luciferase (Minko et al., Mol. Gen. Genet. 262:421-425, 1999) and the amino glycoside phosphotransferase from Acinetobacter baumanii, aphA6 (Bateman and Purton, Mol. Gen. Genet. 263:404-410, 2000).

The vectors described herein may contain modified genes and/or open reading frames containing one or more recombinantly produced features. For example, a gene encoding a protein of interest may be tagged with a useful molecular marker. In some instances, the tag may be an epitope tag or a tag polypeptide. Generally, epitope tags comprise a sufficient number of amino acid residues to provide an epitope against which an antibody can be made, yet is short enough such that it does not interfere with activity of the polypeptide to which it is fused. Preferably a tag is also fairly unique so that an antibody raised to the tag does not substantially cross-react with other epitopes (e.g., a FLAG tag). Other appropriate tags may be used, for example, affinity tags. Affinity tags are appended to proteins so that they can be purified from their crude biological source using an affinity technique. Examples of such tags include, but are not limited to, chitin binding protein (CBP), maltose binding protein (MBP), glutathione-s-transferase (GST) and metal affinity tags (e.g., pol(His). Positioning of tags at the C- and/or N-terminal may be determined based on, for example, protein function. One of skill in the art will recognize that selection of an appropriate tag will be based on multiple factors, including the intended use, the target protein, cost, etc.

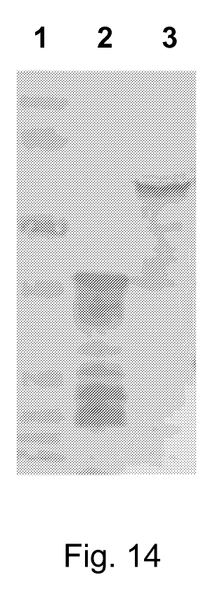

Another example of a modification which may be made to a gene encoding a protein of the present invention is the addition of a cleavage moiety. Typically, the cleavage moiety is a polypeptide of appropriate length to be targeted by a protease. The protease may be naturally occurring in the organism which is intended to be the host for the vectors of the present invention. For example, where the target host is C. reinhardtii, a protein may be engineered to contain an amino acid region recognized by membrane-bound proteases (see, e.g., Hoober et al., Plant Physiol. 1992 July; 99(3): 932-937) or a ClpP protease (NCBI #3053). In other instances, a self-cleaving protease, such as the A2 region (or a functional portion thereof) of Foot and Mouth Disease Virus may be utilized. Halpin, et al., Plant J., 1999; 17(4), 453-459. Typically, cleavage moieties will be utilized for vectors of the present invention which contain fusion proteins. For example, in some instances a vector may comprise a single open reading frame which encodes a fusion protein, a cleavage moiety may be inserted between the sequences encoding portions of the fusion protein (e.g., see FIG. 14A).

Still another modification which may be made to a gene encoding a protein of the present invention is the addition of a secretion signal. Protein secretion is typically conferred by a hydrophobic secretion signal usually located at the N-terminal of the polypeptide which targets the protein to the endoplasmic reticulum and, eventually, the cell membrane. Secretion signals allow for the production and secretion of recombinant proteins in numerous hosts, including NVPOs. One example of a secretion signal which may be utilized in the present invention is the signal from the C. reinhardtii carbonic anhydrase protein. Toguri, et al., Eur. J. Biochem. 1986; 158, 443-450. Many such signals are known in the art, and the selection of an appropriate signal depends on, for example, the host cell and protein folding.

In some instances, the vectors of the present invention will contain elements such as an E. coli or S. cerevisiae origin of replication. Such features, combined with appropriate selectable markers, allows for the vector to be “shuttled” between the target host cell and the bacterial and/or yeast cell. The ability to passage a shuttle vector of the invention in a secondary host may allow for more convenient manipulation of the features of the vector. For example, a reaction mixture containing the vector and putative inserted polynucleotides of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the invention.

A polynucleotide or recombinant nucleic acid molecule of the invention, can be introduced into plant chloroplasts or nucleus using any method known in the art. A polynucleotide can be introduced into a cell by a variety of methods, which are well known in the art and selected, in part, based on the particular host cell. For example, the polynucleotide can be introduced into a plant cell using a direct gene transfer method such as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (Potrykus, Ann. Rev. Plant. Physiol. Plant Mol. Biol. 42:205-225, 1991).

The term “exogenous” is used herein in a comparative sense to indicate that a nucleotide sequence (or polypeptide) being referred to is from a source other than a reference source, or is linked to a second nucleotide sequence (or polypeptide) with which it is not normally associated, or is modified such that it is in a form that is not normally associated with a reference material. For example, a polynucleotide encoding an enzyme is heterologous with respect to a nucleotide sequence of a plant chloroplast, as are the components of a recombinant nucleic acid molecule comprising, for example, a first nucleotide sequence operatively linked to a second nucleotide sequence, as is a mutated polynucleotide introduced into a chloroplast where the mutant polynucleotide is not normally found in the chloroplast.

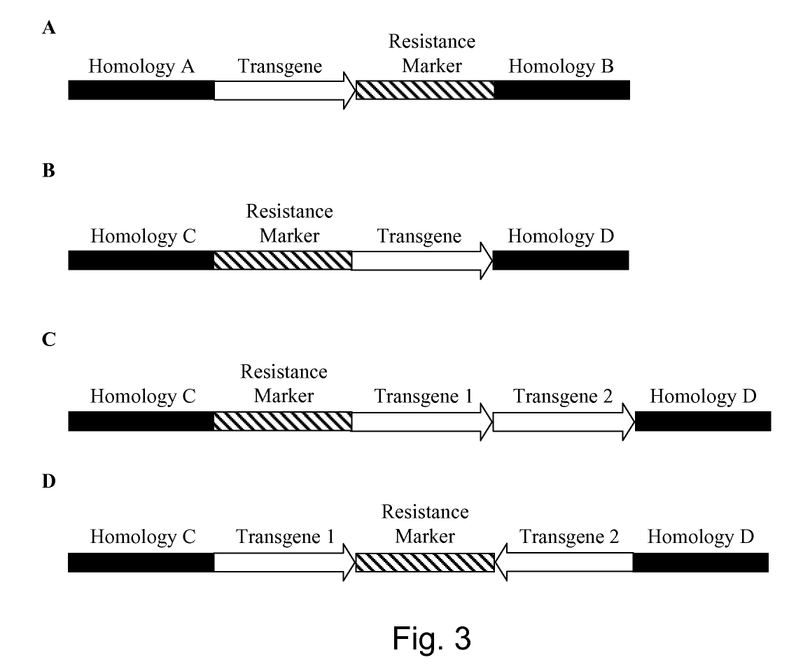

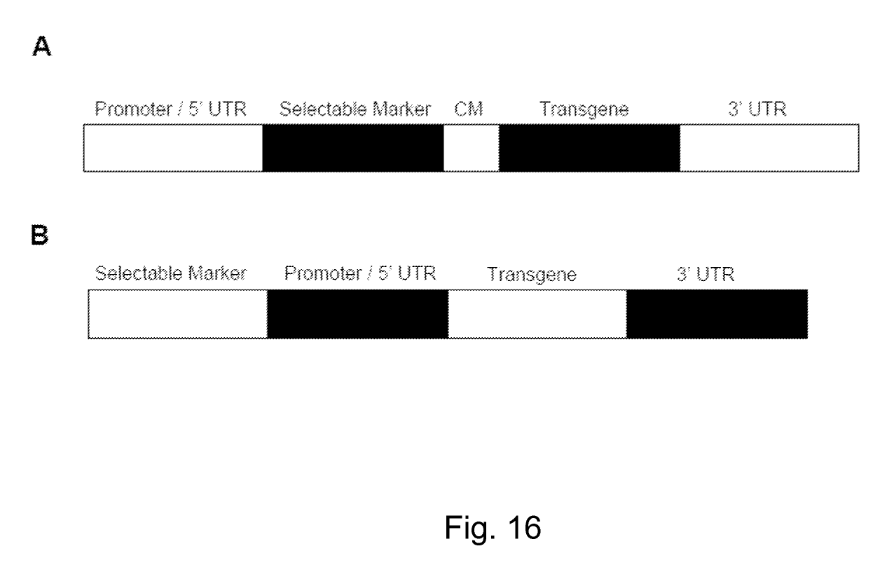

Plastid transformation is a method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. The description herein provides that host cells may be transformed with vectors. One of skill in the art will recognize that such transformation includes transformation with circular or linearized vectors, or linearized portions of a vector. Thus, a host cell comprising a vector may contain the entire vector in the cell (in either circular or linear form), or may contain a linearized portion of a vector of the present invention (e.g., constructs graphically depicted in FIGS. 3 and 16). In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves.

Microprojectile mediated transformation also can be used to introduce a polynucleotide into a plant cell chloroplast (Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PD-1000 particle gun (BioRad; Hercules Calif.). Methods for the transformation using biolistic methods are well known in the art (see, e.g.; Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (Duan et al., Nature Biotech. 14:494-498, 1996; Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, the glass bead agitation method, and the like.

Transformation frequency may be increased by replacement of recessive rRNA or r-protein antibiotic resistance genes with a dominant selectable marker, including, but not limited to the bacterial aadA gene (Svab and Maliga, Proc. Natl. Acad. Sci., USA 90:913-917, 1993). Approximately 15 to 20 cell division cycles following transformation are generally required to reach a homoplastidic state. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein.

A method of the invention can be performed by introducing a recombinant nucleic acid molecule into a chloroplast, wherein the recombinant nucleic acid molecule includes a first polynucleotide, which encodes at least one polypeptide (i.e., 1, 2, 3, 4, or more). In some embodiments, a polypeptide is operatively linked to a second, third, fourth, fifth, sixth, seventh, eighth, ninth, tenth and/or subsequent polypeptide. For example, several enzymes in a hydrocarbon production pathway may be linked, either directly or indirectly, such that products produced by one enzyme in the pathway, once produced, are in close proximity to the next enzyme in the pathway.

For transformation of chloroplasts, one major benefit of the present invention is the utilization of a recombinant nucleic acid construct which contains both a selectable marker and one or more genes of interest. Typically, transformation of chloroplasts is performed by co-transformation of chloroplasts with two constructs: one containing a selectable marker and a second containing the gene(s) of interest. Screening of such transformants is laborious and time consuming for multiple reasons. First, the time required to grow some transformed organisms is lengthy. Second, transformants must be screened both for presence of the selectable marker and for the presence of the gene(s) of interest. Typically, secondary screening for the gene(s) of interest is performed by Southern blot (see, e.g. PCT/US2007/072465).

In chloroplasts, regulation of gene expression generally occurs after transcription, and often during translation initiation. This regulation is dependent upon the chloroplast translational apparatus, as well as nuclear-encoded regulatory factors (see Barkan and Goldschmidt-Clermont, Biochemie 82:559-572, 2000; Zerges, Biochemie 82:583-601, 2000). The chloroplast translational apparatus generally resembles that in bacteria; chloroplasts contain 70S ribosomes; have mRNAs that lack 5′ caps and generally do not contain 3′ poly-adenylated tails (Harris et al., Microbiol. Rev. 58:700-754, 1994); and translation is inhibited in chloroplasts and in bacteria by selective agents such as chloramphenicol.

Some methods of the present invention take advantage of proper positioning of a ribosome binding sequence (RBS) with respect to a coding sequence. It has previously been noted that such placement of an RBS results in robust translation in plant chloroplasts (see U.S. Application 2004/0014174, incorporated herein by reference), and that polypeptides that an advantage of expressing polypeptides in chloroplasts is that the polypeptides do not proceed through cellular compartments typically traversed by polypeptides expressed from a nuclear gene and, therefore, are not subject to certain post-translational modifications such as glycosylation. As such, the polypeptides and protein complexes produced by some methods of the invention can be expected to be produced without such post-translational modification.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition, except that the term “synthetic polynucleotide” as used herein refers to a polynucleotide that has been modified to reflect chloroplast codon usage.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997). Generally, a phosphodiester bond links the nucleotides of a polynucleotide of the present invention; however other bonds, including a thiodiester bond, a phosphorothioate bond, a peptide-like bond and any other bond known in the art may be utilized to produce synthetic polynucleotides (Tam et al., Nucl. Acids Res. 22:977-986, 1994; Ecker and Crooke, BioTechnology 13:351360, 1995).

Any of the products described herein can be prepared by transforming an organism to cause the production and/or secretion by such organism of the product. An organism is considered to be a photosynthetic organism even if a transformation event destroys or diminishes the photosynthetic capability of the transformed organism (e.g., exogenous nucleic acid is inserted into a gene encoding a protein required for photosynthesis).

Fusion Protein Vectors.

In some embodiments of the present invention, a host NVPO nuclear or plastid genome will be targeted for transformation with a construct comprising a fusion protein. Any of the vectors or linearized portions thereof described herein can be modified for nuclear or plastid transformation by incorporating appropriate signals (e.g., a host-cell nuclear origin of replication or plastid origin of replication) or appropriate flanking homology regions (for homologous recombination with the target genome). Some constructs of the present invention are graphically represented in FIG. 14. The construct shown in FIG. 14A comprises at least one regulatory element (“Promoter/5′ UTR” and/or “3′UTR”) and an open reading frame (i.e., a fusion protein) comprising at least two elements (“Selectable Marker” and “Transgene”). In some instances, one or more of the regulatory elements may be endogenous to the organism to be transformed (e.g., the 5′ and 3′ regulatory elements are the flanking homology sections directing insertion of the construct into the target genome). In this figure, the promoter is operably linked to the open reading frame, thus driving expression of the fusion protein. One potential advantage to using such an approach is to prevent recombination events, lowered expression or other effects which might lead to deletion or lowered expression of the transgene (i.e., an enzyme producing an isoprenoid). Additionally, by creating a fusion protein comprising a selectable marker and a transgene, insertion sites may be conserved to create multiply-transformed strains. However, in some instances, a fusion protein may comprise two or more transgenes (e.g., an FPP synthase and a zingiberene synthase).

Also shown in FIG. 14A is the inclusion of an optional cleavage moiety (“CM”), in-frame with the selectable marker and the transgene. As described above, this cleavage moiety, when present, may allow for cleavage of the gene of interest from the selectable marker. Typically, cleavage at the cleavage moiety will result in two functional proteins (e.g., the selectable marker and the transgene). Cleavage may result in a portion of the cleavage moiety remaining on one, both or neither of the flanking polypeptides. Cleavage may occur after or during translation. Thus, in some embodiments, a fusion protein consisting of the selectable marker, the transgene and the intervening cleavage moiety may be present in a host cell. In other embodiments, the fusion protein is cleaved prior to translation of the full-length fusion protein transcript.

Other modifications may be made to a sequence encoding a fusion protein (or any of the other proteins and classes of proteins described herein). One example is a linker polypeptide. Such polypeptides may provide spatial separation of the proteins of interest, allow for proper folding of the portions of a fusion protein, and/or result from recombinant construction of the fusion protein. In another example, a secretion signal may be fused in-frame with one or more portions of the fusion protein (e.g., the transgene, the selectable marker or both). Typically, a secretion signal will be utilized for fusion proteins targeted for expression in a nuclear genome. In other instances, one or more tags may be fused in-frame with one or more portions of the fusion protein. For example, a nucleotide sequence encoding a poly(His) tag may be ligated in-frame with the sequence encoding the transgene and a nucleotide sequence encoding a FLAG tag is ligated in-frame with the sequence encoding the selectable marker. In still other instances, a nucleic acid encoding a secretion signal may be attached in-frame with a portion of the fusion protein. In some instances, a secretion signal will be attached to the transgene. As is apparent, multiple combinations of selectable markers, transgenes, cleavage moieties, signal sequences and/or tags may be combined into a single open reading frame, based on the need of a practitioner. Thus, the simplified versions of the constructs shown in FIG. 14 are not meant to be limiting on the scope of the constructs of the present invention. One of skill in the art will also recognize that such combinations of components may also be utilized in constructs which are used to transform the chloroplast.

FIG. 14B shows another approach to nuclear transformation in which the transforming construct contains a selectable marker and a transgene under control of different regulatory elements. Although not indicated, such constructs may contain cleavage moieties, secretion signals, and/or tags as described above.

Nucleic Acids, Proteins and Enzymes.

The vectors and other nucleic acids disclosed herein can encode polypeptide(s) that promote the production of intermediates, products, precursors, and derivatives of the products described herein. For example, the vectors can encode polypeptide(s) that promote the production of intermediates, products, precursors, and derivatives in the isoprenoid pathway.

The enzymes utilized in practicing the present invention may be encoded by nucleotide sequences derived from any organism, including bacteria, plants, fungi and animals. In some instances, the enzymes are isoprenoid producing enzymes. As used herein, an “isoprenoid producing enzyme” is a naturally or non-naturally occurring enzyme which produces or increases production of an isoprenoid. In some instances, an isoprenoid producing enzyme produces isoprenoids with two phosphate groups (e.g., GPP synthase, FPP synthase, DMAPP synthase). In other instances, isoprenoid producing enzymes produce isoprenoids with zero, one, three or more phosphates or may produce isoprenoids with other functional groups. Non-limiting examples of such enzymes and their sources are shown in Table 1. Polynucleotides encoding enzymes and other proteins useful in the present invention may be isolated and/or synthesized by any means known in the art, including, but not limited to cloning, sub-cloning, and PCR.

TABLE 1
Examples of Synthases for Use in the Present Invention.

| Synthase | Source | NCBI protein ID |
|---|---|---|
| Limonene | M. spicata | 2ONH_A |
| Cineole | S. officinalis | AAC26016 |
| Pinene | A. grandis | AAK83564 |
| Camphene | A. grandis | AAB70707 |
| Sabinene | S. officinalis | AAC26018 |
| Myrcene | A. grandis | AAB71084 |
| Abietadiene | A. grandis | Q38710 |
| Taxadiene | T. brevifolia | AAK83566 |
| FPP | G. gallus | P08836 |
| Amorphadiene | A. annua | AAF61439 |
| Bisabolene | A. grandis | O81086 |
| Diapophytoene | S. aureus | |
| Diapophytoene desaturase | S. aureus | |
| GPPS-LSU | M. spicata | AAF08793 |
| GPPS-SSU | M. spicata | AAF08792 |
| GPPS | A. thaliana | CAC16849 |
| GPPS | C. reinhardtii | EDP05515 |
| FPP | E. coli | NP_414955 |
| FPP | A. thaliana | NP_199588 |
| FPP | A. thaliana | NP_193452 |
| FPP | C. reinhardtii | EDP03194 |
| IPP isomerase | E. coli | NP_417365 |
| IPP isomerase | H. pluvialis | ABB80114 |
| Limonene | L. angustifolia | ABB73044 |
| Monoterpene | S. lycopersicum | AAX69064 |
| Terpinolene | O. basilicum | AAV63792 |
| Myrcene | O. basilicum | AAV63791 |
| Zingiberene | O. basilicum | AAV63788 |
| Myrcene | Q. ilex | CAC41012 |
| Myrcene | P. abies | AAS47696 |
| Myrcene, ocimene | A. thaliana | NP_179998 |
| Myrcene, ocimene | A. thaliana | NP_567511 |
| Sesquiterpene | Z. mays; B73 | AAS88571 |
| Sesquiterpene | A. thaliana | NP_199276 |
| Sesquiterpene | A. thaliana | NP_193064 |
| Sesquiterpene | A. thaliana | NP_193066 |
| Curcumene | P. cablin | AAS86319 |
| Farnesene | M. domestica | AAX19772 |
| Farnesene | C. sativus | AAU05951 |
| Farnesene | C. junos | AAK54279 |
| Farnesene | P. abies | AAS47697 |
| Bisabolene | P. abies | AAS47689 |
| Sesquiterpene | A. thaliana | NP_197784 |
| Sesquiterpene | A. thaliana | NP_175313 |
| GPP Chimera | | |
| GPPS-LSU + SSU fusion | | |
| Geranylgeranyl reductase | A. thaliana | NP_177587 |
| Geranylgeranyl reductase | C. reinhardtii | EDP09986 |
| Chlorophyllidohydrolase | C. reinhardtii | EDP01364 |
| Chlorophyllidohydrolase | A. thaliana | NP_564094 |
| Chlorophyllidohydrolase | A. thaliana | NP_199199 |
| Phosphatase | S. cerevisiae | AAB64930 |
| FPP A118W | G. gallus | |

The synthase may also be botryococcene synthase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, γ-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase.

The vectors of the present invention may be capable of stable transformation of multiple photosynthetic organisms, including, but not limited to, photosynthetic bacteria (including cyanobacteria), cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton. Other vectors of the present invention are capable of stable transformation of C. reinhardtii, D. salina, H. pluvalis, S. dimorphus, D. viridis, or D. tertiolecta.

A vector herein may encode polypeptide(s) having a role in the mevalonate pathway, such as, for example, thiolase, HMG-CoA synthase, HMG-CoA reductase, mevalonate kinase, phosphemevalonate kinase, and mevalonate-5-pyrophosphate decarboxylase. In other embodiments, the polypeptides are enzymes in the non-mevalonate pathway, such as DOXP synthase, DOXP reductase, 4-diphosphocytidyl-2-C-methyl-D-erythritol synthase, 4-diphophocytidyl-2-C-methyl-D-erythritol kinase, 2-C-methyl-D-erythritol 2,4,-cyclodiphosphate synthase, HMB-PP synthase, HMB-PP reductase, or DOXP reductoisomerase.

In other instances, a vector may comprise a nucleotide sequence encoding a polypeptide in an isoprenoid pathway, such as, for example, a synthase-encoding sequence. The synthase may be a C10, C15, C20, C30, or C40 synthase. In some embodiments, the synthase is limonene synthase, 1,8 cineole synthase, α-pinene synthase, camphene synthase, (+)-sabinene synthase, myrcene synthase, abietadiene synthase, taxadiene synthase, farnesyl pyrophosphate synthase, amorphadiene synthase, (E)-α-bisabolene synthase, diapophytoene synthase, or diapophytoene desaturase. Non-limiting examples of synthases and their amino acid sequences are shown in Table 2.

TABLE 2
Protein sequences of synthases

| | | |
|---|---|---|
| SEQ | | |
| ID NO | Synthesized AA Seq | Enzyme |

| | | |
|---|---|---|
| 1 | MVPRRSGNYNPSRWDVNFIQSLLSDYKEDKHVIRASELVTLVKMELEKETDQI | Limonene |
| | RQLELIDDLQRMGLSDHFQNEFKEILSSIYLDHHYYKNPFPKEERDLYSTSLAF | synthase |
| | RLLREHGFQVAQEVFDSFKNEEGEFKESLSDDTRGLLQLYEASFLLTEGETTLE | |
| | SAREFATKFLEEKVNEGGVDGDLLTRIAYSLDIPLHWRIKRPNAPVWIEWYRK | |
| | RPDMNPVVLELAILDLNIVQAQFQEELKESFRWWRNTGFVEKLPFARDRLVE | |
| | CYFWNTGIIEPRQHASARIMMGKVNALITVIDDIYDVYGTLEELEQFTDLIRRW | |
| | DINSIDQLPDYMQLCFLALNNFVDDTSYDVMKEKGVNVIPYLRQSWVDLADK | |
| | YMVEARWFYGGHKPSLEEYLENSWQSISGPCMLTHIFFRVTDSFTKETVDSLY | |
| | KYHDLVRWSSFVLRLADDLGTSVEEVSRGDVPKSLQCYMSDYNASEAEARK | |
| | HVKWLIAEVWKKMNAERVSKDSPFGKDFIGCAVDLGRMAQLMYHNGDGHG | |
| | TQHPIIHQQMTRTLFEPFAGTGENLYFQGSGGGGSDYKDDDDKGTG | |
| 2 | MVPRRTGGYQPTLWDFSTIQLFDSEYKEEKHLMRAAGMIAQVNMLLQEEVD | Cineole |
| | SIQRLELIDDLRRLGISCHFDREIVEILNSKYYTNNEIDESDLYSTALRFKLLRQY | synthase |
| | DFSVSQEVFDCFKNDKGTDFKPSLVDDTRGLLQLYEASFLSAQGEETLHLARD | |
| | FATKFLHKRVLVDKDINLLSSIERALELPTHWRVQMPNARSFIDAYKRRPDMN | |
| | PTVLELAKLDFNMVQAQFQQELKEASRWWNSTGLVHELPFVRDRIVECYYW | |
| | TTGVVERREHGYERIMLTKINALVTTIDDVFDIYGTLEELQLFTTAIQRWDIES | |
| | MKQLPPYMQICYLALFNFVNEMAYDTLRDKGFNSTPYLRKAWVDLVESYLIE | |
| | AKWYYMGHKPSLEEYMKNSWISIGGIPILSHLFFRLTDSIEEEDAESMHKYHDI | |
| | VRASCTILRLADDMGTSLDEVERGDVPKSVQCYMNEKNASEEEAREHVRSLI | |
| | DQTWKMMNKEMMTSSFSKYFVQVSANLARMAQWIYQHESDGFGMQHSLV | |
| | NKMLRGLLFDRYEGTGENLYFQGSGGGGSDYKDDDDKGTG | |
| 3 | MVPRRMGDFHSNLWDDDVIQSLPTAYEEKSYLERAEKLIGEVENMFNSMSLE | Pinene |
| | DGELMSPLNDLIQRLWIVDSLGRLGIHRHFKDEIKSALDYVYSYWGENGIGCG | synthase |
| | RESAVTDLNSTALGFRTLRLHGYPVSSDVFKAFKGQNGQFSCSENIQTDEEIRG | |
| | VLNLFRASLIAFPGEKIMDEAEIFSTKYLKEALQKIPVSSLSREIGDVLEYGWHT | |
| | YLPRLEARNYIHVFGQDTENTKSYVKSKKLLELAKLEFNIFQSLQKRELESLVR | |
| | WWKESGFPEMTFCRHRHVEYYTLASCIAFEPQHSGFRLGFAKTCHLITVLDD | |
| | MYDTFGTVDELELFTATMKRWDPSSIDCLPEYMKGVYIAVYDTVNEMAREA | |
| | EEAQGRDTLTYAREAWEAYIDSYMQEARWIATGYLPSFDEYYENGKVSCGH | |
| | RISALQPILTMDIPFPDHILKEVDFPSKLNDLACAILRLRGDTRCYKADRARGEE | |
| | ASSISCYMKDNPGVSEEDALDHINAMISDVIKGLNWELLKPDINVPISAKKHAF | |
| | DIARAFHYGYKYRDGYSVANVETKSLVTRTLLESVPLGTGENLYFQGSGGGG | |
| | SDYKDDDDKGTG | |
| 4 | MVPRRVGNYHSNLWDDDFIQSLISTPYGAPDYRERADRLIGEVKDIMFNFKSL | Camphene |
| | EDGGNDLLQRLLLVDDVERLGIDRHFKKEIKTALDYVNSYWNEKGIGCGRES | synthase |
| | VVTDLNSTALGLRTLRLHGYTVSSDVLNVFKDKNGQFSSTANIQIEGEIRGVL | |
| | NLFRASLVAFPGEKVMDEAETFSTKYLREALQKIPASSILSLEIRDVLEYGWHT | |
| | NLPRLEARNYMDVFGQHTKNKNAAEKLLELAKLEFNIFHSLQERELKHVSRW | |
| | WKDSGSPEMTFCRHRHVEYYALASCIAFEPQHSGFRLGFTKMSHLITVLDDM | |
| | YDVFGTVDELELFTATIKRWDPSAMECLPEYMKGVYMMVYHTVNEMARVA | |
| | EKAQGRDTLNYARQAWEACFDSYMQEAKWIATGYLPTFEEYLENGKVSSAH | |
| | RPCALQPILTLDIPFPDHILKEVDFPSKLNDLICIILRLRGDTRCYKADRARGEEA | |
| | SSISCYMKDNPGLTEEDALNHINFMIRDAIRELNWELLKPDNSVPITSKKHAFD | |
| | ISRVWHHGYRYRDGYSFANVETKSLVMRTVIEPVPLGTGENLYFQGSGGGGS | |
| | DYKDDDDKGTG | |
| 5 | MVPRRSGDYQPSLWDFNYIQSLNTPYKEQRHFNRQAELIMQVRMLLKVKME | Sabinene |
| | AIQQLELIDDLQYLGLSYFFQDEIKQILSSIHNEPRYFHNNDLYFTALGFRILRQ | synthase |
| | HGFNVSEDVFDCFKIEKCSDFNANLAQDTKGMLQLYEASFLLREGEDTLELAR | |
| | RFSTRSLREKFDEGGDEIDEDLSSWIRHSLDLPLHWRVQGLEARWFLDAYARR | |
| | PDMNPLIFKLAKLNFNIVQATYQEELKDISRWWNSSCLAEKLPFVRDRIVECFF | |
| | WAIAAFEPHQYSYQRKMAAVIITFITIIDDVYDVYGTIEELELLTDMIRRWDNK | |
| | SISQLPYYMQVCYLALYNFVSERAYDILKDQHFNSIPYLQRSWVSLVEGYLKE | |
| | AYWYYNGYKPSLEEYLNNAKISISAPTIISQLYFTLANSIDETAIESLYQYHNIL | |
| | YLSGTILRLADDLGTSQHELERGDVPKAIQCYMNDTNASEREAVEHVKFLIRE | |
| | AWKEMNTVTTASDCPFTDDLVAAAANLARAAQFIYLDGDGHGVQHSEIHQQ | |
| | MGGLLFQPYVGTGENLYFQGSGGGGSDYKDDDDKGTG | |
| 6 | MVPRRIGDYHSNIWDDDFIQSLSTPYGEPSYQERAERLIVEVKKIFNSMYLDDG | Myrcene |
| | RLMSSFNDLMQRLWIVDSVERLGIARHFKNEITSALDYVFRYWEENGIGCGRD | synthase |
| | SIVTDLNSTALGFRTLRLHGYTVSPEVLKAFQDQNGQFVCSPGQTEGEIRSVLN | |
| | LYRASLIAFPGEKVMEEAEIFSTRYLKEALQKIPVSALSQEIKFVMEYGWHTNL | |
| | PRLEARNYIDTLEKDTSAWLNKNAGKKLLELAKLEFNIFNSLQQKELQYLLR | |
| | WWKESDLPKLTFARHRHVEFYTLASCIAIDPKHSAFRLGFAKMCHLVTVLDDI | |
| | YDTFGTIDELELFTSAIKRWNSSEIEHLPEYMKCVYMVVFETVNELTREAEKT | |
| | QGRNTLNYVRKAWEAYFDSYMEEAKWISNGYLPMFEEYHENGKVSSAYRV | |
| | ATLQPILTLNAWLPDYILKGIDFPSRFNDLASSFLRLRGDTRCYKADRDRGEEA | |
| | SCISCYMKDNPGSTEEDALNHINAMVNDIIKELNWELLRSNDNIPMLAKKHAF | |
| | DITRALHHLYIYRDGFSVANKETKKLVMETLLESMLFGTGENLYFQGSGGGG | |
| | SDYKDDDDKGTG | |
| 7 | MVPQSAEKNDSLSSSTLVKREFPPGFWKDDLIDSLTSSHKVAASDEKRIETLIS | Abietadiene |
| | EIKNMFRCMGYGETNPSAYDTAWVARIPAVDGSDNPHFPETVEWILQNQLKD | synthase |
| | GSWGEGFYFLAYDRILATLACIITLTLWRTGETQVQKGIEFFRTQAGKMEDEA | |
| | DSHRPSGFEIVFPAMLKEAKILGLDLPYDLPFLKQIIEKREAKLKRIPTDVLYAL | |
| | PTTLLYSLEGLQEIVDWQKIMKLQSKDGSFLSSPASTAAVFMRTGNKKCLDFL | |
| | NFVLKKFGNHVPCHYPLDLFERLWAVDTVERLGIDRHFKEEIKEALDYVYSH | |
| | WDERGIGWARENPVPDIDDTAMGLRILRLHGYNVSSDVLKTFRDENGEFFCFL | |
| | GQTQRGVTDMLNVNRCSHVSFPGETIMEEAKLCTERYLRNALENVDAFDKW | |
| | AFKKNIRGEVEYALKYPWHKSMPRLEARSYIENYGPDDVWLGKTVYMMPYI | |
| | SNEKYLELAKLDFNKVQSIHQTELQDLRRWWKSSGFTDLNFTRERVTEIYFSP | |
| | ASFIFEPEFSKCREVYTKTSNFTVILDDLYDAHGSLDDLKLFTESVKRWDLSLV | |
| | DQMPQQMKICFVGFYNTFNDIAKEGRERQGRDVLGYIQNVWKVQLEAYTKE | |
| | AEWSEAKYVPSFNEYIENASVSIALGTVVLISALFTGEVLTDEVLSKIDRESRFL | |
| | QLMGLTGRLVNDTKTYQAERGQGEVASAIQCYMKDHPKISEEEALQHVYSV | |
| | MENALEELNREFVNNKIPDIYKRLVFETARIMQLFYMQGDGLTLSHDMEIKEH | |
| | VKNCLFQPVAGTGENLYFQGSGGGGSDYKDDDDKGTG | |
| 8 | MVPSSSTGTSKVVSETSSTIVDDIPRLSANYHGDLWHHNVIQTLETPFRESSTY | Taxadiene |
| | QERADELVVKIKDMFNALGDGDISPSAYDTAWVARVATISSDGSEKPRFPQAL | synthase |
| | NWVFNNQLQDGSWGIESHFSLCDRLLNTTNSVIALSVWKTGHSQVQQGAEFI | |
| | AENLRLLNEEDELSPDFQIIFPALLQKAKALGINLPYDLPFIKYLSTTREARLTD | |
| | VSAAADNIPANMLNALEGLEEVIDWNKIMRFQSKDGSFLSSPASTACVLMNT | |
| | GDEKCFTFLNNLLDKFGGCVPCMYSIDLLERLSLVDNIEHLGIGRHFKQEIKGA | |
| | LDYVYRHWSERGIGWGRDSLVPDLNTTALGLRTLRMHGYNVSSDVLNNFKD | |
| | ENGRFFSSAGQTHVELRSVVNLFRASDLAFPDERAMDDARKFAEPYLREALA | |
| | TKISTNTKLFKEIEYVVEYPWHMSIPRLEARSYIDSYDDNYVWQRKTLYRMPS | |
| | LSNSKCLELAKLDFNIVQSLHQEELKLLTRWWKESGMADINFTRHRVAEVYF | |
| | SSATFEPEYSATRIAFTKIGCLQVLFDDMADIFATLDELKSFTEGVKRWDTSLL | |
| | HEIPECMQTCFKVWFKLMEEVNNDVVKVQGRDMLAHIRKPWELYFNCYVQE | |
| | REWLEAGYIPTFEEYLKTYAISVGLGPCTLQPILLMGELVKDDVVEKVHYPSN | |
| | MFELVSLSWRLTNDTKTYQAEKVRGQQASGIACYMKDNPGATEEDAIKHICR | |
| | VVDRALKEASFEYFKPSNDIPMGCKSFIFNLRLCVQIFYKFIDGYGIANEEIKDY | |
| | IRKVYIDPIQVGTGENLYFQGSGGGGSDYKDDDDKGTG | |
| 9 | MVPHKFTGVNAKFQQPALRNLSPVVVEREREEFVGFFPQIVRDLTEDGIGHPE | FPP |
| | VGDAVARLKEVLQYNAPGGKCNRGLTVVAAYRELSGPGQKDAESLRCALAV | synthase |
| | GWCIELFQAFFLVADDIMDQSLTRRGQLCWYKKEGVGLDAINDSFLLESSVY | |
| | RVLKKYCRQRPYYVHLLELFLQTAYQTELGQMLDLITAPVSKVDLSHFSEERY | |
| | KAIVKYKTAFYSFYLPVAAAMYMVGIDSKEEHENAKAILLEMGEYFQIQDDY | |
| | LDCFGDPALTGKVGTDIQDNKCSWLVVQCLQRVTPEQRQLLEDNYGRKEPEK | |
| | VAKVKELYEAVGMRAAFQQYEESSYRRLQELIEKHSNRLPKEIFLGLAQKIYK | |
| | RQKGTGENLYFQGSGGGGSDYKDDDDKGTG | |
| 10 | MVPSLTEEKPIRPIANFPPSIWGDQFLIYEKQVEQGVEQIVNDLKKEVRQLLKE | Amorphadiene |
| | ALDIPMKHANLLKLIDEIQRLGIPYHFEREIDHALQCIYETYGDNWNGDRSSL | synthase |
| | WFRLMRKQGYYVTCDVFNNYKDKNGAFKQSLANDVEGLLELYEATSMRVP | |
| | GEIILEDALGFTRSRLSIMTKDAFSTNPALFTEIQRALKQPLWKRLPRIEAAQYI | |
| | PFYQQQDSHNKTLLKLAKLEFNLLQSLHKEELSHVCKWWKAFDIKKNAPCLR | |
| | DRIVECYFWGLGSGYEPQYSRARVFFTKAVAVITLIDDTYDAYGTYEELKIFTE | |
| | AVERWSITCLDTLPEYMKPIYKLFMDTYTEMEEFLAKEGRTDLFNCGKEFVKE | |
| | FVRNLMVEAKWANEGHIPTTEEHDPVVIITGGANLLTTTCYLGMSDIFTKESV | |
| | EWAVSAPPLFRYSGILGRRLNDLMTHKAEQERKHSSSSLESYMKEYNVNEEY | |
| | AQTLIYKEVEDVWKDINREYLTTKNIPRPLLMAVIYLCQFLEVQYAGKDNFTR | |
| | MGDEYKHLIKSLLVYPMSIGTGENLYFQGSGGGGSDYKDDDDKGTG | |
| 12 | MVPAGVSAVSKVSSLVCDLSSTSGLIRRTANPHPNVWGYDLVHSLKSPYIDSS | Bisabolene |
| | YRERAEVLVSEIKAMLNPAITGDGESMITPSAYDTAWVARVPAIDGSARPQFP | synthase |
| | QTVDWILKNQLKDGSWGIQSHFLLSDRLLATLSCVLVLLKWNVGDLQVEQGI | |
| | EFIKSNLELVKDETDQDSLVTDFEIIFPSLLREAQSLRLGLPYDLPYIHLLQTKR | |
| | QERLAKLSREEIYAVPSPLLYSLEGIQDIVEWERIMEVQSQDGSFLSSPASTACV | |
| | FMHTGDAKCLEFLNSVMIKFGNFVPCLYPVDLLERLLIVDNIVRLGIYRHFEKE | |
| | IKEALDYVYRHWNERGIGWGRLNPIADLETTALGFRLLRLHRYNVSPAIFDNF | |
| | KDANGKFICSTGQFNKDVASMLNLYRASQLAFPGENILDEAKSFATKYLREAL | |
| | EKSETSSAWNNKQNLSQEIKYALKTSWHASVPRVEAKRYCQVYRPDYARIAK | |
| | CVYKLPYVNNEKFLELGKLDFNIIQSIHQEEMKNVTSWFRDSGLPLFTFARERP | |
| | LEFYFLVAAGTYEPQYAKCRFLFTKVACLQTVLDDMYDTYGTLDELKLFTEA | |
| | VRRWDLSFTENLPDYMKLCYQIYYDIVHEVAWEAEKEQGRELVSFFRKGWE | |
| | DYLLGYYEEAEWLAAEYVPTLDEYIKNGITSIGQRILLLSGVLIMDGQLLSQEA | |
| | LEKVDYPGRRVLTELNSLISRLADDTKTYKAEKARGELASSIECYMKDHPECT | |
| | EEEALDHIYSILEPAVKELTREFLKPDDVPFACKKMLFEETRVTMVIFKDGDGF | |
| | GVSKLEVKDHIKECLIEPLPLGTGENLYFQGSGGGGSDYKDDDDKGTG | |
| 13 | MVPTMMNMNFKYCHKIMKKHSKSFSYAFDLLPEDQRKAVWAIYAVCRKIDD | Diapophytoene |
| | SIDVYGDIQFLNQIKEDIQSIEKYPYEHHHFQSDRRIMMALQHVAQHKNIAFQS | synthase |
| | FYNLIDTVYKDQHFTMFETDAELFGYCYGVAGTVGEVLTPILSDHETHQTYD | |
| | VARRLGESLQLINILRDVGEDFDNERIYFSKQRLKQYEVDIAEVYQNGVNNHY | |
| | IDLWEYYAAIAEKDFQDVMDQIKVFSIEAQPIIELAARIYIEILDEVRQANYTLH | |
| | ERVFVDKRKKAKLFHENKGTGENLYFQGSGGGGSDYKDDDDKGTG | |
| 14 | MVPKIAVIGAGVTGLAAAARIASQGHEVTIFEKNNNVGGRMNQLKKDGFTFD | Diapophytoene |
| | MGPTIVMMPDVYKDVFTACGKNYEDYIELRQLRYIYDVYFDHDDRITVPTDL | desaturase |
| | AELQQMLESIEPGSTHGFMSFLTDVYKKYEIARRYFLERTYRKPSDFYNMTSL | |
| | VQGAKLKTLNHADQLIEHYIDNEKIQKLLAFQTLYIGIDPKRGPSLYSIIPMIEM | |
| | MFGVHFIKGGMYGMAQGLAQLNKDLGVNIELNAEIEQIIIDPKFKRADAIKVN | |
| | GDIRKFDKILCTADFPSVAESLMPDFAPIKKYPPHKIADLDYSCSAFLMYIGIDI | |
| | DVTDQVRLHNVIFSDDFRGNIEEIFEGRLSYDPSIYVYVPAVADKSLAPEGKTG | |
| | IYVLMPTPELKTGSGIDWSDEALTQQIKEIIYRKLATIEVFEDIKSHIVSETIFTPN | |
| | DFEQTYHAKFGSAFGLMPTLAQSNYYRPQNVSRDYKDLYFAGASTHPGAGVP | |
| | IVLTSAKITVDEMIKDIERGVGTGENLYFQGSGGGGSDYKDDDDKGTG | |
| 15 | MVPAFDFDGYMLRKAKSVNKALEAAVQMKEPLKIHESMRYSLLAGGKRVRP | GPPS- |
| | MLCIAACELVGGDESTAMPAACAVEMIHTMSLMHDDLPCMDNDDLRRGKPT | LSU |
| | NHMAFGESVAVLAGDALLSFAFEHVAAATKGAPPERIVRVLGELAVSIGSEGL | synthase |
| | VAGQVVDVCSEGMAEVGLDHLEFIHHHKTAALLQGSVVLGAILGGGKEEEV | |
| | AKLRKFANCIGLLFQVVDDILDVTKSSKELGKTAGKDLVADKTTYPKLIGVEK | |
| | SKEFADRLNREAQEQLLHFHPHRAAPLIALANYIAYRDNGTGENLYFQGSGG | |
| | GGSDYKDDDDKGTG | |
| 16 | MVPSQPYWAAIEADIERYLKKSITIRPPETVFGPMHHLTFAAPATAASTLCLAA | GPPS- |
| | CELVGGDRSQAMAAAAAIHLVHAAAYVHEHLPLTDGSRPVSKPAIQHKYGPN | SSU |
| | VELLTGDGIVPFGFELLAGSVDPARTDDPDRILRVIIEISRAGGPEGMISGLHRE | synthase |
| | EEIVDGNTSLDFIEYVCKKKYGEMHACGAACGAILGGAAEEEIQKLRNFGLYQ | |
| | GTLRGMMEMKNSHQLIDENIIGKLKELALEELGGFHGKNAELMSSLVAEPSLY | |
| | AAGTGENLYFQGSGGGGSDYKDDDDKGTG | |
| 17 | MVPLLSNKLREMVLAEVPKLASAAEYFFKRGVQGKQFRSTILLLMATALDVR | GPPS |
| | VPEALIGESTDIVTSELRVRQRGIAEITEMIHVASLLHDDVLDDADTRRGVGSL | |
| | NVVMGNKMSVLAGDFLLSRACGALAALKNTEVVALLATAVEHLVTGETMEI | |
| | TSSTEQRYSMDYYMQKTYYKTASLISNSCKAVAVLTGQTAEVAVLAFEYGRN | |
| | LGLAFQLIDDILDFTGTSASLGKGSLSDIRHGVITAPILFAMEEFPQLREVVDQV | |
| | EKDPRNVDIALEYLGKSKGIQRARELAMEHANLAAAAIGSLPETDNEDVKRSR | |
| | RALIDLTHRVITRNKGTGENLYFQGSGGGGSDYKDDDDKGTG | |
| 18 | MVPVVSERLRHSVTTGIPALKTAAEYFFRRGIEGKRLRPTLALLMSSALSPAAP | GPPS |
| | SPEYLQVDTRPAAEHPHEMRRRQQRLAEIAELIHVASLLHDDVIDDAQTRRGV | |
| | LSLNTSVGNKTAILAGDFLLARASVTLASLRNSEIVELMSQVLEHLVSGEIMQ | |
| | MTATSEQLLDLEHYLAKTYCKTASLMANSSRSVAVLAGAAPEVCDMAWSYG | |
| | RHLGIAFQVVDDLLDLTGSSSVLGKPALNDMRSGLATAPVLFAAQEEPALQA | |
| | LILRRFKHDGDVTKAMSLIERTQGLRRAEELAAQHAKAAADMIRCLPTAQSD | |
| | HAEIAREALIQITHRVLTRKKGTGENLYFQGSGGGGSDYKDDDDKGTG | |
| 19 | MVPDFPQQLEACVKQANQALSRFIAPLPFQNTPVVETMQYGALLGGKRLRPF | FPP |
| | LVYATGHMFGVSTNTLDAPAAAVECIHAYSLIHDDLPAMDDDDLRRGLPTCH | synthase |
| | VKFGEANAILAGDALQTLAFSILSDADMPEVSDRDRISMISELASASGIAGMCG | |
| | GQALDLDAEGKHVPLDALERIHRHKTGALIRAAVRLGALSAGDKGRRALPVL | |
| | DKYAESIGLAFQVQDDILDVVGDTATLGKRQGADQQLGKSTYPALLGLEQAR | |
| | KKARDLIDDARQSLKQLAEQSLDTSALEALADYIIQRNKGTGENLYFQGSGGG | |
| | GSDYKDDDDKGTG | |
| 20 | MVPSVSCCCRNLGKTIKKAIPSHHLHLRSLGGSLYRRRIQSSSMETDLKSTFLN | FPP |
| | VYSVLKSDLLHDPSFEFTNESRLWVDRMLDYNVRGGKLNRGLSVVDSFKLLK | synthase |
| | QGNDLTEQEVFLSCALGWCIEWLQAYFLVLDDIMDNSVTRRGQPCWFRVPQ | |
| | VGMVAINDGILLRNHIHRILKKHFRDKPYYVDLVDLFNEVELQTACGQMIDLI | |
| | TTFEGEKDLAKYSLSIHRRIVQYKTAYYSFYLPVACALLMAGENLENHIDVKN | |
| | VLVDMGIYFQVQDDYLDCFADPETLGKIGTDIEDFKCSWLVVKALERCSEEQT | |
| | KILYENYGKPDPSNVAKVKDLYKELDLEGVFMEYESKSYEKLTGAIEGHQSK | |
| | AIQAVLKSFLAKIYKRQKGTGENLYFQGSGGGGSDYKDDDDKGTG | |
| 21 | MVPADLKSTFLDVYSVLKSDLLQDPSFEFTHESRQWLERMLDYNVRGGKLNR | FPP |
| | GLSVVDSYKLLKQGQDLTEKETFLSCALGWCIEWLQAYFLVLDDIMDNSVTR | synthase |
| | RGQPCWFRKPKVGMIAINDGILLRNHIHRILKKHFREMPYYVDLVDLFNEVEF | |
| | QTACGQMIDLITTFDGEKDLSKYSLQIHRRIVEYKTAYYSFYLPVACALLMAG | |
| | ENLENHTDVKTVLVDMGIYFQVQDDYLDCFADPETLGKIGTDIEDFKCSWLV | |
| | VKALERCSEEQTKILYENYGKAEPSNVAKVKALYKELDLEGAFMEYEKESYE | |
| | KLTKLIEAHQSKAIQAVLKSFLAKIYKRQKGTGENLYFQGSGGGGSDYKDDD | |
| | DKGTG | |
| 22 | MVPSGEPTPKKMKATYVHDRENFTKVYETLRDELLNDDCLSPAGSPQAQAA | FPP |
| | QEWFKEVNDYNVPGGKLNRGMAVYDVLASVKGPDGLSEDEVFKANALGWC | synthase |
| | IEWLQAFFLVADDIMDGSITRRGQPCWYKQPKVGMIACNDYILLECCIYSILKR | |
| | HFRGHAAYAQLMDLFHETTFQTSHGQLLDLTTAPIGSVDLSKYTEDNYLRIVT | |
| | YKTAYYSFYLPVACGMVLAGITDPAAFDLAKNICVEMGQYFQIQDDYLDCYG | |
| | DPEVIGKIGTDIEDNKCSWLVCTALKIATEEQKEVIKANYGHKEAESVAAIKAL | |
| | YVELGIEQRFKDYEAASYAKLEGTISEQTLLPKAVFTSLLAKIYKRKKGTGENL | |
| | YFQGSGGGGSDYKDDDDKGTG | |
| 23 | MVPQTEHVILLNAQGVPTGTLEKYAAHTADTRLHLAFSSWLFNAKGQLLVTR | IPP |
| | RALSKKAWPGVWTNSVCGHPQLGESNEDAVIRRCRYELGVEITPPESIYPDFR | isomerase |
| | YRATDPSGIVENEVCPVFAARTTSALQINDDEVMDYQWCDLADVLHGIDATP | |
| | WAFSPWMVMQATNREARKRLSAFTQLKGTGENLYFQGSGGGGSDYKDDDD | |
| | KGTG | |
| 24 | MVPLRSLLRGLTHIPRVNSAQQPSCAHARLQFKLRSMQLLAENRTDHMRGAS | IPP |
| | TWAGGQSQDELMLKDECILVDADDNITGHASKLECHKFLPHQPAGLLHRAFS | isomerase |
| | VFLFDDQGRLLLQQRARSKITFPSVWANTCCSHPLHGQTPDEVDQQSQVADG | |
| | TVPGAKAAAIRKLEHELGIPAHQLPASAFRFLTRLHYCAADVQPAATQSALW | |
| | GEHEMDYILFIRANVTLAPNPDEVDEVRYVTQEELRQMMQPDNGLQWSPWF | |
| | RIIAARFLERWWADLDAALNTDKHEDWGTVHHINEAGTGENLYFQGSGGGG | |
| | SDYKDDDDKGTG | |
| 25 | MVPRRSGNYNPTAWDFNYIQSLDNQYKKERYSTRHAELTVQVKKLLEEEME | Limonene |
| | AVQKLELIEDLKNLGISYPFKDNIQQILNQIYNEHKCCHNSEVEEKDLYFTALR | synthase |
| | FRLLRQQGFEVSQEVFDHFKNEKGTDFKPNLADDTKGLLQLYEASFLLREAED | |
| | TLELARQFSTKLLQKKVDENGDDKIEDNLLLWIRRSLELPLHWRVQRLEARGF | |
| | LDAYVRRPDMNPIVFELAKLDFNITQATQQEELKDLSRWWNSTGLAEKLPFA | |
| | RDRVVESYFWAMGTFEPHQYGYQRELVAKIIALATVVDDVYDVYGTLEELEL | |
| | FTDAIRRWDRESIDQLPYYMQLCFLTVNNFVFELAHDVLKDKSFNCLPHLQRS | |
| | WLDLAEAYLVEAKWYHSRYTPSLEEYLNIARVSVTCPTIVSQMYFALPIPIEKP | |
| | VIEIMYKYHDILYLSGMLLRLPDDLGTASFELKRGDVQKAVQCYMKERNVPE | |
| | NEAREHVKFLIREASKQINTAMATDCPFTEDFAVAAANLGRVANFVYVDGDG | |
| | FGVQHSKIYEQIGTLMFEPYPGTGENLYFQGSGGGGSDYKDDDDKGTG | |
| 26 | MVPRRSGNYKPTMWDFQFIQSVNNLYAGDKYMERFDEVKKEMKKNLMMM | Monoterpene |
| | VEGLIEELDVKLELIDNLERLGVSYHFKNEIMQILKSVHQQITCRDNSLYSTAL | synthase |
| | KFRLLRQHGFHISQDIFNDFKDMNGNVKQSICNDTKGLLELYEASFLSTECETT | |
| | LKNFTEAHLKNYVYINHSCGDQYNNIMMELVVHALELPRHWMMPRLETRW | |
| | YISIYERMPNANPLLLELAKLDFNIVQATHQQDLKSLSRWWKNMCLAEKLSFS | |
| | RNRLVENLFWAVGTNFEPQHSYFRRLITKIIVFVGIIDDIYDVYGKLDELELFTL | |
| | AVQRWDTKAMEDLPYYMQVCYLALINTTNDVAYEVLRKHNINVLPYLTKSW | |
| | TDLCKSYLQEARWYYNGYKPSLEEYMDNGWISIAVPMVLAHALFLVTDPITK | |
| | EALESLTNYPDIIRCSATIFRLNDDLGTSSDELKRGDVPKSIQCYMNEKGVSEE | |
| | EAREHIRFLIKETWKFMNTAHHKEKSLFCETFVEIAKNIATTAHCMYLKGDSH | |
| | GIQNTDVKNSISNILFHPIIIGTGENLYFQGSGGGGSDYKDDDDKGTG | |
| 27 | MVPRRSGNYEPSAWDFNYLQSLNNYHHKEERYLRRQADLIEKVKMILKEEK | Terpinolene |
| | MEALQQLELIDDLRNLGLSYCFDDQINHILTTIYNQHSCFHYHEAATSEEANLY | synthase |
| | FTALGFRLLREHGFKVSQEVFDRFKNEKGTDFRPDLVDDTQGLLQLYEASFLL | |
| | REGEDTLEFARQFATKFLQKKVEEKMIEEENLLSWTLHSLELPLHWRIQRLEA | |
| | KWFLDAYASRPDMNPIIFELAKLEFNIAQALQQEELKDLSRWWNDTGIAEKLP | |
| | FARDRIVESHYWAIGTLEPYQYRYQRSLIAKIIALTTVVDDVYDVYGTLDELQ | |
| | LFTDAIRRWDIESINQLPSYMQLCYLAIYNFVSELAYDIFRDKGFNSLPYLHKS | |
| | WLDLVEAYFQEAKWYHSGYTPSLEQYLNIAQISVASPAILSQIYFTMAGSIDKP | |
| | VIESMYKYRHILNLSGILLRLPDDLGTASDELGRGDLAKAMQCYMKERNVSE | |
| | EEARDHVRFLNREVSKQMNPARAADDCPFTDDFVVAAANLGRVADFMYVE | |
| | GDGLGLQYPAIHQHMAELLFHPYAGTGENLYFQGSGGGGSDYKDDDDKGTG | |
| 28 | MVPRRSGNYQPSAWDFNYIQSLNNNHSKEERHLERKAKLIEEVKMLLEQEMA | Myrcene |
| | AVQQLELIEDLKNLGLSYLFQDEIKIILNSIYNHHKCFHNNHEQCIHVNSDLYF | synthase |
| | VALGFRLFRQHGFKVSQEVFDCFKNEEGSDFSANLADDTKGLLQLYEASYLV | |
| | TEDEDTLEMARQFSTKILQKKVEEKMIEKENLLSWTLHSLELPLHWRIQRLEA | |
| | KWFLDAYASRPDMNPIIFELAKLEFNIAQALQQEELKDLSRWWNDTGIAEKLP | |
| | FARDRIVESHYWAIGTLEPYQYRYQRSLIAKIIALTTVVDDVYDVYGTLDELQ | |
| | LFTDAIRRWDIESINQLPSYMQLCYLAIYNFVSELAYDIFRDKGFNSLPYLHKS | |
| | WLDLVEAYFVEAKWFHDGYTPTLEEYLNNSKITIICPAIVSEIYFAFANSIDKTE | |
| | VESIYKYHDILYLSGMLARLPDDLGTSSFEMKRGDVAKAIQCYMKEHNASEE | |
| | EAREHIRFLMREAWKHMNTAAAADDCPFESDLVVGAASLGRVANFVYVEGD | |
| | GFGVQHSKIHQQMAELLFYPYQGTGENLYFQGSGGGGSDYKDDDDKGTG | |
| 29 | MVPRRSANYQASIWDDNFIQSLASPYAGEKYAEKAEKLKTEVKTMIDQTRDE | Zingiberene |
| | LKQLELIDNLQRLGICHHFQDLTKKILQKIYGEERNGDHQHYKEKGLHFTALR | synthase |
| | FRILRQDGYHVPQDVFSSFMNKAGDFEESLSKDTKGLVSLYEASYLSMEGETI | |
| | LDMAKDFSSHHLHKMVEDATDKRVANQIIHSLEMPLHRRVQKLEAIWFIQFY | |
| | ECGSDANPTLVELAKLDFNMVQATYQEELKRLSRWYEETGLQEKLSFARHRL | |
| | AEAFLWSMGIIPEGHFGYGRMHLMKIGAYITLLDDIYDVYGTLEELQVLTEIIE | |
| | RWDINLLDQLPEYMQIFFLYMFNSTNELAYEILRDQGINVISNLKGLWVELSQ | |
| | CYFKEATWFHNGYTPTTEEYLNVACISASGPVILFSGYFTTTNPINKHELQSLE | |
| | RHAHSLSMILRLADDLGTSSDEMKRGDVPKAIQCFMNDTGCCEEEARQHVKR | |
| | LIDAEWKKMNKDILMEKPFKNFCPTAMNLGRISMSFYEHGDGYGGPHSDTKK | |
| | KMVSLFVQPMNITIGTGENLYFQGSGGGGSDYKDDDDKGTG | |
| 30 | MVPRRSANYQPSIWNHDYIESLRIEYVGETCTRQINVLKEQVRMMLHKVVNP | Myrcene |
| | LEQLELIEILQRLGLSYHFEEEIKRILDGVYNNDHGGDTWKAENLYATALKFR | synthase |
| | LLRQHGYSVSQEVFNSFKDERGSFKACLCEDTKGMLSLYEASFFLIEGENILEE | |
| | ARDFSTKHLEEYVKQNKEKNLATLVNHSLEFPLHWRMPRLEARWFINIYRHN | |
| | QDVNPILLEFAELDFNIVQAAHQADLKQVSTWWKSTGLVENLSFARDRPVEN | |
| | FFWTVGLIFQPQFGYCRRMFTKVFALITTIDDVYDVYGTLDELELFTDVVERW | |
| | DINAMDQLPDYMKICFLTLHNSVNEMALDTMKEQRFHIIKYLKKAWVDLCR | |
| | YYLVEAKWYSNKYRPSLQEYIENAWISIGAPTILVHAYFFVTNPITKEALDCLE | |
| | EYPNIIRWSSIIARLADDLGTSTDELKRGDVPKAIQCYMNETGASEEGAREYIK | |
| | YLISATWKKMNKDRAASSPFSHIFIEIALNLARMAQCLYQHGDGHGLGNRET | |
| | KDRILSLLIQPIPLNKDGTGENLYFQGSGGGGSDYKDDDDKGTG | |
| 31 | MVPRRIGDYHSNLWNDDFJQSLTTPYGAPSYIERADRLISEVKEMFNRMCMED | Myrcene |
| | GELMSPLNDLIQRLWTVDSVERLGIDRHFKNEIKASLDYVYSYWNEKGJGCGR | synthase |
| | QSVVTDLNSTALGLRILRQHGYTVSSEVLKVFEEENGQFACSPSQTEGEIRSFL | |
| | NLYRASLIAFPGEKVMEEAQIFSSRYLKEAVQKJPVSGLSREIGDVLEYGWHTN | |
| | LPRWEARNYMDVFGQDTNTSFNKNKMQYMNTEKILQLVKLEFNIFHSLQQRE | |
| | LQCLLRWWKESGLPQLTFARHRHVEFYTLASCIACEPKHSAFRLGFAKMCHL | |
| | VTVLDDVYDTFGKMDELELFTAAVKRWDLSETERLPEYMKGLYVVVFETVN | |
| | ELAQEAEKTQGRNTLNYVRKAWEAYFDSYMKEAEWISTGYLPTFEEYCENG | |
| | KVSSAYRVAALQPILTLDVQLPDDILKGIDFPSRFNDLASSFLRLRGDTRCYEA | |
| | DRARGEEASCISCYMKDNPGSTEEDALNHINAMINDIIRELNWEFLKPDSNIPM | |
| | PARKHAFDITRALHHLYIYRDGFSVANKETKNLVEKTLLESMLFGTGENLYFQ | |
| | GSGGGGSDYKDDDDKGTG | |
| 32 | MVPRRSANYQPSRWDHHHLLSVENKFAKDKRVRERDLLKEKVRKMLNDEQ | Myrcene, |
| | KTYLDQLEFIDDLQKLGVSYHFEAEIDNILTSSYKKDRTNIQESDLHATALEFR | ocimene |
| | LFRQHGFNVSEDVFDVFMENCGKFDRDDIYGLISLYEASYLSTKLDKNLQIFIR | synthase |
| | PFATQQLRDFVDTHSNEDFGSCDMVEIVVQALDMPYYWQMRRLSTRWYIDV | |
| | YGKRQNYKNLVVVEFAKIDFNIVQAIHQEELKNVSSWWMETGLGKQLYFAR | |
| | DRIVENYFWTIGQIQEPQYGYVRQTMTKINALLTTIDDIYDIYGTLEELQLFTV | |
| | AFENWDINRLDELPEYMRLCFLVIYNEVNSIACEILRTKNINVIPFLKKSWTDV | |
| | SKAYLVEAKWYKSGHKPNLEEYMQNARISISSPTIFVHFYCVFSDQLSIQVLET | |
| | LSQHQQNVVRCSSSVFRLANDLVTSPDELARGDVCKSIQCYMSETGASEDKA | |
| | RSHVRQMINDLWDEMNYEKMAHSSSILHHDFMETVINLARMSQCMYQYGD | |
| | GHGSPEKAKIVDRVMSLLFNPIPLDGTGENLYFQGSGGGGSDYKDDDDKGTG | |
| 33 | MVPRRSANYQPSLWQHEYLLSLGNTYVKEDNVERVTLLKQEVSKMLNETEG | Myrcene, |
| | LLEQLELIDTLQRLGVSYHFEQEIKKTLTNVHVKNVRAHKNRIDRNRWGDLY | ocimene |
| | ATALEFRLLRQHGFSIAQDVFDGNIGVDLDDKDIKGILSLYEASYLSTRIDTKL | synthase |
| | KESIYYTTKRLRKFVEVNKNETKSYTLRRMVIHALEMPYHRRVGRLEARWYI | |
| | EVYGERHDMNPILLELAKLDFNFVQAIHQDELKSLSSWWSKTGLTKHLDFVR | |
| | DRITEGYFSSVGVMYEPEFAYHRQMLTKVFMLITTIDDIYDIYGTLEELQLFTTI | |
| | VEKWDVNRLEELPNYMKLCFLCLVNEINQIGYFVLRDKGFNVIPYLKESWAD | |
| | MCTTFLKEAKWYKSGYKPNFEEYMQNGWISSSVPTILLHLFCLLSDQTLDILG | |
| | SYNHSVVRSSATILRLANDLATSSEELARGDTMKSVQCHMHETGASEAESRA | |
| | YIQGIIGVAWDDLNMEKKSCRLHQGFLEAAANLGRVAQCVYQYGDGHGCPD | |
| | KAKTVNHVRSLLVHPLPLNGTGENLYFQGSGGGGSDYKDDDDKGTG | |
| 34 | MVPASPPAHRSSKAADEELPKASSTFHPSLWGSFFLTYQPPTAPQRANMKERA | Sesquiterpene |
| | EVLRERVRKVLKGSTTDQLPETVNLILTLQRLGLGYYYENEIDKLLHQIYSNSD | synthase |
| | YNVKDLNLVSQRFYLLRKNGYDVPSDVFLSFKTEEGGFACAAADTRSLLSLY | |
| | NAAYLRKHGEEVLDEAISSTRLRLQDLLGRLLPESPFAKEVSSSLRTPLFRRVGI | |
| | LEARNYIPIYETEATRNEAVLELAKLNFNLQQLDFCEELKHCSAWWNEMIAKS | |
| | KLTFVRDRIVEEYFWMNGACYDPPYSLSRIILTKITGLITIIDDMFDTHGTTEDC | |
| | MKFAEAFGRWDESAIHLLPEYMKDFYILMLETFQSFEDALGPEKSYRVLYLK | |
| | QAMERLVELYSKEIKWRDDDYVPTMSEHLQVSAETIATIALTCSAYAGMGDM | |
| | SIRKETFEWALSFPQFIRTFGSFVRLSNDVVSTKREQTKDHSPSTVHCYMKEHG | |
| | TTMDDACEKIKELIEDSWKDMLEQSLALKGLPKVVPQLVFDFSRTTDNMYRD | |
| | RDALTSSEALKEMIQLLFVEPIPEGTGENLYFQGSGGGGSDYKDDDDKGTG | |
| 35 | MVPEALGNFDYESYTNFTKLPSSQWGDQFLKFSIADSDFDVLEREIEVLKPKV | Sesquiterpene |
| | RENIFVSSSTDKDAMKKTILSIHFLDSLGLSYHFEKEIEESLKHAFEKIEDLIADE | synthase |
| | NKLHTISTIFRVFRTYGYYMSSDVFKIFKGDDGKFKESLIEDVKGMLSFYEAVH | |
| | FGTTTDHILDEALSFTLNHLESLATGRRASPPHISKLIQNALHIPQHRNIQALVA | |
| | REYISFYEHEEDHDETLLKLAKLNFKFLQLHYFQELKTITMWWTKLDHTSNLP | |
| | PNFRERTVETWFAALMMYFEPQFSLGRIMSAKLYLVITFLDDACDTYGSISEV | |
| | ESLADCLERWDPDYMENLQGHMKTAFKFVMYLFKEYEEILRSQGRSFVLEK | |
| | MIEEFKIIARKNLELVKWARGGHVPSFDEYIESGGAEIGTYATIACSIMGLGEIG | |
| | KKEAFEWLISRPKLVRILGAKTRLMDDIADFEEDMEKGYTANALNYYMNEHG | |
| | VTKEEASRELEKMNGDMNKIVNEECLKITTMPRRILMQSVNYARSLDVLYTA | |
| | DDVYNHREGKLKEYMRLLLVDPILLGTGENLYFQGSGGGGSDYKDDDDKGTG | |
| 36 | MVPESQTTFKYESLAFTKLSHCQWTDYFLSVPIDESELDVITREIDILKPEVMEL | Sesquiterpene |
| | LSSQGDDETSKRKVLLIQLLLSLGLAFHFENEIKNILEHAFRKIDDITGDEKDLS | synthase |
| | TISIMFRVFRTYGHNLPSSVFKRFTGDDGKFQQSLTEDAKGILSLYEAAHLGTT | |
| | TDYILDEALKFTSSHLKSLLAGGTCRPHILRLIRNTLYLPQRWNMEAVIAREYI | |
| | SFYEQEEDHDKMLLRLAKLNFKLLQLHYIKELKSFIKWWMELGLTSKWPSQF | |
| | RERIVEAWLAGLMMYFEPQFSGGRVIAAKFNYLLTILDDACDHYFSIHELTRL | |
| | VACVERWSPDGIDTLEDISRSVFKLMLDVFDDIGKGVRSEGSSYHLKEMLEEL | |
| | NTLVRANLDLVKWARGIQTAGKEAYEWVRSRPRLIKSLAAKGRLMDDITDFD | |
| | SDMSNGFAANAINYYMKQFVVTKEEAILECQRMIVDINKTINEELLKTTSVPG | |
| | RVLKQALNFGRLLELLYTKSDDIYNCSEGKLKEYIVTLLIDPIRLGTGENLYFQ | |
| | GSGGGGSDYKDDDDKGTG | |
| 37 | MVPESQTKFDYESLAFTKLSHSQWTDYFLSVPIDDSELDAITREIDIIKPEVRKL | Sesquiterpene |
| | LSSKGDDETSKRKVLLIQSLLSLGLAFHFENEIKDILEDAFRRIDDITGDENDLS | synthase |
| | TISIMFRVFRTYGHNLPSSVFKRFTGDDGKFERSLTEDAKGILSLYEAAHLGTT | |
| | TDYILDEALEFTSSHLKSLLVGGMCRPHILRLIRNTLYLPQRWNMEAVIAREYI | |
| | SFYEQEEDHDKMLLRLAKLNFKLLQLHYIKELKTFIKWWMELGLTSKWPSQF | |
| | RERIVEAWLAGLMMYFEPQFSGGRVIAAKFNYLLTILDDACDHYFSIPELTRL | |
| | VDCVERWNHDGIHTLEDISRIIFKLALDVFDDIGRGVRSKGCSYYLKEMLEEL | |
| | KILVRANLDLVKWARGNQLPSFEEHVEVGGIALTTYATLMYSFVGMGEAVG | |
| | KEAYEWVRSRPRLIKSLAAKGRLMDDITDFEVKIINLFFDLLLFVFGTGENLYF | |
| | QGSGGGGSDYKDDDDKGTG | |
| 38 | MVPAAFTANAVDMRPPVITIHPRSKDIFSQFSLDDKLQKQYAQGIEALKEEAR | Curcumene |
| | SMLMAAKSAKVMILIDTLERLGLGYHFEKEIEEKLEAIYKKEDGDDYDLFTTA | synthase |
| | LRFRLLRQHQRRVPCSVFDKFMNKEGKFEEEPLISDVEGLLSLYDAAYLQIHG | |
| | EHILQEALIFTTHHLTRIEPQLDDHSPLKLKLNRALEFPFYREIPIIYAHFYISVYE | |
| | RDDSRDEVLLKMAKLSYNFLQNLYKKELSQLSRWWNKLELIPNLPYIRDSVA | |
| | GAYLWAVALYFEPQYSDVRMAIAKLIQIAAAVDDTYDNYATIREAQLLTEAL | |
| | ERLNVHEIDTLPDYMKIVYRFVMSWSEDFERDATIKEQMLATPYFKAEMKKL | |
| | GRAYNQELKWVMERQLPSFEEYMKNSEITSGVYIMFTVISPYLNSATQKNIDW | |
| | LLSQPRLASSTAIVMRCCNDLGSNQRESKGGEVMTSLDCYMKQHGASKQETI | |
| | SKFKLIIEDEWKNLNEEWAATTCLPKVMVEIFRNYARIAGFCYKNNGDAYTSP | |
| | KIVQQCFDALFVNPLRIGTGENLYFQGSGGGGSDYKDDDDKGTG | |
| 39 | MVPEFRVHLQADNEQKJFQNQMKPEPEASYLINQRRSANYKPNIWKNDFLDQ | Farnesene |
| | SLISKYDGDEYRKLSEKLIEEVKIYISAETMDLVAKLELIDSVRKLGLANLFEKE | synthase |
| | IKEALDSIAAIESDNLGTRDDLYGTALHFKILRQHGYKVSQDIFGRFMDEKGTL | |
| | ENHHFAHLKGMLELFEASNLGFEGEDILDEAKASLTLALRDSGHICYPDSNLS | |
| | RDVVHSLELPSHRRVQWFDVKWQINAYEKDICRVNATLLELAKLNFNVVQA | |
| | QLQKNLREASRWWANLGFADNLKFARDRLVECFSCAVGVAFEPEHSSFRICL | |
| | TKVINLVLIIDDVYDIYGSEEELKHFTNAVDRWDSRETEQLPECMKMCFQVLY | |
| | NTTCEIAREIEEENGWNQVLPQLTKVWADFCKALLVEAEWYNKSHIPTLEEYL | |
| | RNGCISSSVSVLLVHSFFSITHEGTKEMADFLHKNEDLLYNISLIVRLNNDLGTS | |
| | AAEQERGDSPSSIVCYMREVNASEETARKNIKGMIDNAWKKVNGKCFTTNQV | |
| | PFLSSFMNNATNMARVAHSLYKDGDGFGDQEKGPRTHILSLLFQPLVNGTGE | |
| | NLYFQGSGGGGSDYKDDDDKGTG | |
| 40 | MVPSSNVSAIPNSFELIRRSAQFQASVWGDYFLSYHSLPPEKGNKVMEKQTEE | Farnesene |
| | LKEEIKMELVSTTKDEPEKLRLIDLIQRLGVCYHFENEINNILQQLHHITITSEKN | synthase |
| | GDDNPYNMTLCFRLLRQQGYNVSSEPFDRFRGKWESSYDNNVEELLSLYEAS | |
| | QLRMQGEEALDEAFCFATAQLEAIVQDPTTDPMVAAEIRQALKWPMYKNLPR | |
| | LKARHHIGLYSEKPWRNESLLNFAKMDFNKLQNLHQTEIAYISKWWDDYGFA | |
| | EKLSFARNRIVEGYFFALGIFFEPQLLTARLIMTKVIAIGSMLDDIYDVYGTFEE | |
| | LKLLTLALERWDKSETKQLPNYMKMYYEALLDVFEEIEQEMSQKETETTPYCI | |
| | HHMKEATKELGRVFLVEATWCKEGYTPKVEEYLDIALISFGHKLLMVTALLG | |
| | MGSHMATQQIVQWITSMPNILKASAVICRLMNDIVSHKFEQERGHVASAIECY | |
| | MEQNHLSEYEALIALRKQIDDLWKDMVENYCAVITEDEVPRGVLMRVLNLTR | |
| | LFNVIYKDGDGYTQSHGSTKAHIKSLLVDSVPLGTGENLYFQGSGGGGSDYK | |
| | DDDDKGTG | |
| 41 | MVPKDMSIPLLAAVSSSTEETVRPIADFHPTLWGNHFLKSAADVETIDAATQE | Farnesene |
| | QHAALKQEVRRMITTTANKLAQKLHMIDAVQRLGVAYHFEKEIEDELGKVSH | synthase |
| | DLDSDDLYVVSLRFRLFRQQGVKISCDVFDKFKDDEGKFKESLINDIRGMLSL | |
| | YEAAYLAIRGEDILDEAIVFTTTHLKSVISISDHSHANSNLAEQIRHSLQIPLRKA | |
| | AARLEARYFLDIYSRDDLHDETLLKFAKLDFNILQAAHQKEASIMTRWWNDL | |
| | GFPKKVPYARDRIIETYIWMLLGVSYEPNLAFGRIFASKVVCMITTIDDTFDAY | |
| | GTFEELTLFTEAVTRWDIGLIDTLPEYMKFIVKALLDIYREAEEELAKEGRSYGI | |
| | PYAKQMMQELIILYFTEAKWLYKGYVPTFDEYKSVALRSIGLRTLAVASFVDL | |
| | GDFIATKDNFECILKNAKSLKATETIGRLMDDIAGYKFEQKRGHNPSAVECYK | |
| | NQHGVSEEEAVKELLLEVANSWKDINEELLNPTTVPLPMLQRLLYFARSGHFI | |
| | YDDGHDRYTHSLMMKRQVALLLTEPLAIGTGENLYFQGSGGGGSDYKDDDD | |
| | KGTG | |
| 42 | MVPDLAVEIAMDLAVDDVERRVGDYHSNLWDDDFIQSLSTPYGASSYRERAE | Farnesene |
| | RLVGEVKEMFTSISIEDGELTSDLLQRLWMVDNVERLGISRHFENEIKAAIDYV | synthase |
| | YSYWSDKGIVRGRDSAVPDLNSIALGFRTLRLHGYTVSSDVFKVFQDRKGEFA | |
| | CSAIPTEGDIKGVLNLLRASYIAFPGEKVMEKAQTFAATYLKEALQKIQVSSLS | |
| | REIEYVLEYGWLTNFPRLEARNYIDVFGEEICPYFKKPCIMVDKLLELAKLEFN | |
| | LFHSLQQTELKHVSRWWKDSGFSQLTFTRHRHVEFYTLASCIAIEPKHSAFRL | |
| | GFAKVCYLGIVLDDIYDTFGKMKELELFTAAIKRWDPSTTECLPEYMKGVYM | |
| | AFYNCVNELALQAEKTQGRDMLNYARKAWEALFDAFLEEAKWISSGYLPTF | |
| | EEYLENGKVSFGYRAATLQPILTLDIPLPLHILQQIDFPSRFNDLASSILRLRGDI | |
| | CGYQAERSRGEEASSISCYMKDNPGSTEEDALSHINAMISDNINELNWELLKP | |
| | NSNVPISSKKHAFDILRAFYHLYKYRDGFSIAKIETKNLVMRTVLEPVPMGTGE | |
| | NLYFQGSGGGGSDYKDDDDKGTG | |
| 43 | MVPTSVSVESGTVSCLSSNNLIRRTANPHPNIWGYDFVHSLKSPYTHDSSYRER | Bisabolene |
| | AETLISEIKVMLGGGELMMTPSAYDTAWVARVPSIDGSACPQFPQTVEWILKN | synthase |
| | QLKDGSWGTESHFLLSDRLLATLSCVLALLKWKVADVQVEQGIEFIKRNLQAI | |
| | KDERDQDSLVTDFEIIFPSLLKEAQSLNLGLPYDLPYIRLLQTKRQERLANLSM | |
| | DKIHGGTLLSSLEGIQDIVEWETIMDVQSQDGSFLSSPASTACVFMHTGDMKC | |
| | LDFLNNVLTKFGSSVPCLYPVDLLERLLIVDNVERLGIDRHFEKEIKEALDYVY | |
| | RHWNDRGIGWGRLSPIADLETTALGFRLLRLHRYNVSPVVLDNFKDADGEFF | |
| | CSTGQFNKDVASMLSLYRASQLAFPEESILDEAKSFSTQYLREALEKSETFSSW | |
| | NHRQSLSEEIKYALKTSWHASVPRVEAKRYCQVYRQDYAHLAKSVYKLPKV | |
| | NNEKILELAKLDFNIIQSIHQKEMKNVTSWFRDSGLPLFTFARERPLEFYFLIAG | |
| | GTYEPQYAKCRFLFTKVACLQTVLDDMYDTYGTPSELKLFTEAVRRWDLSFT | |
| | ENLPDYMKLCYKIYYDIVHEVAWEVEKEQGRELVSFFRKGWEDYLLGYYEE | |
| | AEWLAAEYVPTLDEYIKNGITSIGQRILLLSGVLIMEGQLLSQEALEKVDYPGR | |
| | RVLTELNSLISRLADDTKTYKAEKARGELASSIECYMKDHPGCQEEEALNHIY | |
| | GILEPAVKELTREFLKADHVPFPCKKMLFDETRVTMVIFKDGDGFGISKLEVK | |
| | DHIKECLIEPLPLGTGENLYFQGSGGGGSDYKDDDDKGTG | |
| 44 | MVPGSEVNRPLADFPANIWEDPLTSFSKSDLGTETFKEKHSTLKEAVKEAFMS | Sesquiterpene |
| | SKANPIENIKFIDALCRLGVSYHFEKDIVEQLDKSFDCLDFPQMVRQEGCDLYT | synthase |
| | VGIIFQVFRQFGFKLSADVFEKFKDENGKFKGHLVTDAYGMLSLYEAAQWGT | |
| | HGEDIIDEALAFSRSHLEEISSRSSPHLAIRIKNALKHPYHKGISRIETRQYISYYE | |
| | EEESCDPTLLEFAKIDFNLLQILHREELACVTRWHHEMEFKSKVTYTRHRITEA | |
| | YLWSLGTYFEPQYSQARVITTMALILFTALDDMYDAYGTMEELELFTDAMDE | |
| | WLPVVPDEIPIPDSMKFIYNVTVEFYDKLDEELEKEGRSGCGFHLKKSLQKTA | |
| | NGYMQEAKWLKKDYIATFDEYKENAILSSGYYALIAMTFVRMTDVAKLDAF | |
| | EWLSSHPKIRVASEIISRFTDDISSYEFEHKREHVATGIDCYMQQFGVSKERAV | |
| | EVMGNIVSDAWKDLNQELMRPHVFPFPLLMRVLNLSRVIDVFYRYQDAYTNP | |
| | KLLKEHIVSLLIETIPIGTGENLYFQGSGGGGSDYKDDDDKGTG | |
| 45 | MVPEAIRVFGLKLGSKLSIHSQTNAFPAFKLSRFPLTSFPGKHAHLDPLKATTH | Sesquiterpene |
| | PLAFDGEENNREFKNLGPSEWGHQFLSAHVDLSEMDALEREIEALKPKVRDM | synthase |
| | LISSESSKKKILFLYLLVSLGLAYHFEDEIKESLEDGLQKIEEMMASEDDLRFKG | |
| | DNGKFKECLAKDAKGILSLYEAAHMGTTTDYILDEALSFTLTYMESLAASGTC | |
| | KINLSRRIRKALDQPQHKNMEIIVAMKYIQFYEEEEDCDKTLLKFAKLNFKFLQ | |
| | LHYLQELKILSKWYKDQDFKSKLPPYFRDRLVECHFASLTCFEPKYARARIFLS | |
| | KIFTVQIFIDDTCDRYASLGEVESLADTIERWDPDDHAMDGLPDYLKSVVKFV | |
| | FNTFQEFERKCKRSLRINLQVAKWVKAGHLPSFDEYLDVAGLELAISFTFAGIL | |
| | MGMENVCKPEAYEWLKSRDKLVRGVITKVRLLNDIFGYEDDMRRGYVTNSI | |
| | NCYKKQYGVTEEEAIRKLHQIVADGEKMMNEEFLKPINVPYQVPKVVILDTL | |
| | RAANVSYEKDDEFTRPGEHLKNCITSIYFDLGTGENLYFQGSGGGGSDYKDD | |
| | DDKGTG | |
| 46 | MVPTTTLSSNLNSQFMQVYETLKSELIHDPLFEFDDDSRQWVERMIDYTVPGG | GPP |
| | KMVRGYSVVDSYQLLKGEELTEEEAFLACALGWCTEWFQAFILLHDDMMDG | Chimera |
| | SHTRRGQPCWFRLPEVGAVAINDGVLLRNHVHRILKKHFQGKAYYVHLVDLF | synthase |
| | NETEFQTISGQMIDLITTLVGEKDLSKYSLSIHRRIVQYKTAYYSFYLPVACALL | |
| | MFGEDLDKHVEVKNVLVEMGTYFQVQDDYLDCFGAPEVIGKIGTDIEDFKCS | |
| | WLVVKALELANEEQKKTLHENYGKKDPASVAKVKEVYHTLNLQAVFEDYE | |
| | ATSYKKLITSIENHPSKAVQAVLKSFLGKIYKRQKGTGENLYFQGSGGGGSDY | |
| | KDDDDKGTG | |
| 47 | MVPSQPYWAAIEADIERYLKKSITIRPPETVFGPMHHLTFAAPATAASTLCLAA | GPPS- |
| | CELVGGDRSQAMAAAAAIHLVHAAAYVHEHLPLTDGSRPVSKPAIQHKYGPN | LSU + SSU |
| | VELLTGDGIVPFGFELLAGSVDPARTDDPDRILRVIIEISRAGGPEGMISGLHRE | fusion |
| | EEIVDGNTSLDFIEYVCKKKYGEMHACGAACGAILGGAAEEEIQKLRNFGLYQ | |
| | GTLRGMMEMKNSHQLIDENIIGKLKELALEELGGFHGKNAELMSSLVAEPSLY | |
| | AASSNNLGIEGRFDFDGYMLRKAKSVNKALEAAVQMKEPLKIHESMRYSLLA | |
| | GGKRVRPMLCIAACELVGGDESTAMPAACAVEMIHTMSLMHDDLPCMDND | |
| | DLRRGKPTNHMAFGESVAVLAGDALLSFAFEHVAAATKGAPPERIVRVLGEL | |
| | AVSIGSEGLVAGQVVDVCSEGMAEVGLDHLEFIHHHKTAALLQGSVVLGAIL | |
| | GGGKEEEVAKLRKFANCIGLLFQVVDDILDVTKSSKELGKTAGKDLVADKTT | |
| | YPKLIGVEKSKEFADRLNREAQEQLLHFHPHRAAPLIALANYIAYRDNGTGEN | |
| | LYFQGSGGGGSDYKDDDDKGTG | |
| 48 | MVPVTAARATPKLSNRKLRVAVIGGGPAGGAAAETLAQGGIETILIERKMDN | Geranyl |
| | CKPCGGAIPLCMVGEFNLPLDIIDRRVTKMKMISPSNIAVDIGRTLKEHEYIGM | geranyl |
| | VRREVLDAYLRERAEKSGATVINGLFLKMDHPENWDSPYTLHYTEYDGKTG | reductase |
| | ATGTKKTMEVDAVIGADGANSRVAKSIDAGDYDYAIAFQERIRIPDEKMTYY | |
| | EDLAEMYVGDDVSPDFYGWVFPKCDHVAVGTGTVTHKGDIKKFQLATRNRA | |
| | KDKILGGKIIRVEAHPIPEHPRPRRLSKRVALVGDAAGYVTKCSGEGIYFAAKS | |
| | GRMCAEAIVEGSQNGKKMIDEGDLRKYLEKWDKTYLPTYRVLDVLQKVFYR | |
| | SNPAREAFVEMCNDEYVQKMTFDSYLYKRVAPGSPLEDIKLAVNTIGSLVRA | |
| | NALRREIEKLSVGTGENLYFQGSGGGGSDYKDDDDKGTG | |
| 49 | MVPVAVIGGGPSGACAAETLAKGGVETFLLERKLDNCKPCGGAIPLCMVEEF | Geranyl- |
| | DLPMEIIDRRVTKMKMISPSNREVDVGKTLSETEWIGMCRREVFDDYLRNRA | geranyl |
| | QKLGANIVNGLFMRSEQQSAEGPFTIHYNSYEDGSKMGKPATLEVDMIIGADG | reductase |
| | ANSRIAKEIDAGEYDYAIAFQERIRIPDDKMKYYENLAEMYVGDDVSPDFYG | |
| | WVFPKYDHVAVGTGTVVNKTAIKQYQQATRDRSKVKTEGGKIIRVEAHPIPE | |
| | HPRPRRCKGRVALVGDAAGYVTKCSGEGIYFAAKSGRMAAEAIVEGSANGT | |
| | KMCGEDAIRVYLDKWDRKYWTTYKVLDILQKVFYRSNPAREAFVELCEDSY | |
| | VQKMTFDSYLYKTVVPGNPLDDVKLLVRTVSSILRSNALRSVNSKSVNVSFGS | |
| | KANEERVMAAGTGENLYFQGSGGGGSDYKDDDDKGTG | |
| 50 | MVPAMAVPLDVVITYPSSGAAAYPVLVMYNGFQAKAPWYRGIVDHVSSWG | Chlorophyllido- |
| | YTVVQYTNGGLFPIVVDRVELTYLEPLLTWLETQSADAKSPLYGRADVSRLG | hydrolase |
| | TMGHSRGGKLAALQFAGRTDVSGCVLFDPVDGSPMTPESADYPSATKALAA | |
| | AGRSAGLVGAAITGSCNPVGQNYPKFWGALAPGSWQMVLSQAGHMQFART | |
| | GNPFLDWSLDRLCGRGTMMSSDVITYSAAFTVAWFEGIFRPAQSQMGISNFKT | |
| | WANTQVAARSITFDIKPMQSPQGTGENLYFQGSGGGGSDYKDDDDKGTG | |
| 51 | MVPAPPKPVRITCPTVAGTYPVVLFFHGFYLRNYFYSDVLNHIASHGYILVAP | Chlorophyllido- |
| | QLCKLLPPGGQVEVDDAGSVINWASENLKAHLPTSVNANGKYTSLVGHSRGG | hydrolase |
| | KTAFAVALGHAATLDPSITFSALIGIDPVAGTNKYIRTDPHILTYKPESFELDIPV | |
| | AVVGTGLGPKWNNVMPPCAPTDLNHEEFYKECKATKAHFVAADYGHMDML | |
| | DDDLPGFVGFMAGCMCKNGQRKKSEMRSFVGGIVVAFLKYSLWGEKAEIRLI | |
| | VKDPSVSPAKLDPSPELEEASGIFVGTGENLYFQGSGGGGSDYKDDDDKGTG | |
| 52 | MVPATPVEEGDYPVVMLLHGYLLYNSFYSQLMLHVSSHGFILIAPQLYSIAGP | Chlorophyllido- |
| | DTMDEIKSTAEIMDWLSVGLNHFLPAQVTPNLSKFALSGHSRGGKTAFAVAL | hydrolase |
| | KKFGYSSNLKISTLIGIDPVDGTGKGKQTPPPVLAYLPNSFDLDKTPILVIGSGL | |
| | GETARNPLFPPCAPPGVNHREFFRECQGPAWHFVAKDYGHLDMLDDDTKGIR | |
| | GKSSYCLCKNGEERRPMRRFVGGLVVSFLKAYLEGDDRELVKIKDGCHEDVP | |
| | VEIQEFEVIMGTGENLYFQGSGGGGSDYKDDDDKGTG | |
| 53 | MVPSHKKKNVIFFVTDGMGPASLSMARSFNQHVNDLPIDDILTLDEHFIGSSRT | Phosphatase |
| | RSSDSLVTDSAAGATAFACALKSYNGAIGVDPHHRPCGTVLEAAKLAGYLTG | |
| | LVVTTRITDATPASFSSHVDYRWQEDLIATHQLGEYPLGRVVDLLMGGGRSH | |
| | FYPQGEKASPYGHHGARKDGRDLIDEAQSNGWQYVGDRKNFDSLLKSHGEN | |
| | VTLPFLGLFADNDIPFEIDRDEKEYPSLKEQVKVALGALEKASNEDKDSNGFFL | |
| | MVEGSRIDHAGHQNDPASQVREVLAFDEAFQYVLEFAENSDTETVLVSTSDH | |
| | ETGGLVTSRQVTASYPQYVWYPQVLANATHSGEFLKRKLVDFVHEHKGASS | |
| | KIENFIKHEILEKDLGIYDYTDSDLETLIHLDDNANAIQDKLNDMVSFRAQIGW | |
| | TTHGHSAVDVNIYAYANKKATWSYVLNNLQGNHENTEVGQFLENFLELNLN | |
| | EVTDLIRDTKHTSDFDATEIASEVQHYDEYYHELTNGTGENLYFQGSGGGGSD | |
| | YKDDDDKGTG | |
| 54 | MVPHKFTGVNAKFQQPALRNLSPVVVEREREEFVGFFPQIVRDLTEDGIGHPE | FPP |
| | VGDAVARLKEVLQYNAPGGKCNRGLTVVAAYRELSGPGQKDAESLRCALAV | A118W |
| | GWCIELFQAFFLVWDDIMDQSLTRRGQLCWYKKEGVGLDAINDSFLLESSVY | |
| | RVLKKYCRQRPYYVHLLELFLQTAYQTELGQMLDLITAPVSKVDLSHFSEERY | |
| | KAIVKYKTAFYSFYLPVAAAMYMVGIDSKEEHENAKAILLEMGEYFQIQDDY | |
| | LDCFGDPALTGKVGTDIQDNKCSWLVVQCLQRVTPEQRQLLEDNYGRKEPEK | |
| | VAKVKELYEAVGMRAAFQQYEESSYRRLQELIEKHSNRLPKEIFLGLAQKIYK | |
| | RQKGTGENLYFQGSGGGGSDYKDDDDKGTG | |

One or more codons of an encoding polynucleotide can be biased to reflect chloroplast and/or nuclear codon usage. Most amino acids are encoded by two or more different (degenerate) codons, and it is well recognized that various organisms utilize certain codons in preference to others. Such preferential codon usage, which also is utilized in chloroplasts, is referred to herein as “chloroplast codon usage”. The codon bias of Chlamydomonas reinhardtii has been reported. See U.S. Application 2004/0014174. Examples of nucleic acids encoding isoprenoid biosynthetic enzymes which are biased for expression in C. reinhardtii are provided in Tables 5-8. Percent identity to the native sequence (in the organism from which the sequence was isolated) may be about 50%, about 60%, about 70%, about 80%, about 90% or higher. Some vectors of the present invention comprise one or more of the nucleic provided in Table 5 and/or nucleic acids with about 70% identity thereto.

One example of an algorithm that is suitable for determining percent sequence identity or sequence similarity between nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnology Information. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915). In addition to calculating percent sequence identity, the BLAST algorithm also can perform a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “biased,” when used in reference to a codon, means that the sequence of a codon in a polynucleotide has been changed such that the codon is one that is used preferentially in the target which the bias is for, e.g., alga cells, chloroplasts. A polynucleotide that is biased for chloroplast codon usage can be synthesized de novo, or can be genetically modified using routine recombinant DNA techniques, for example, by a site directed mutagenesis method, to change one or more codons such that they are biased for chloroplast codon usage. Chloroplast codon bias can be variously skewed in different plants, including, for example, in alga chloroplasts as compared to tobacco. Generally, the chloroplast codon bias selected reflects chloroplast codon usage of the plant which is being transformed with the nucleic acids of the present invention. For example, where C. reinhardtii is the host, the chloroplast codon usage is biased to reflect alga chloroplast codon usage (about 74.6% AT bias in the third codon position).

One method of the invention can be performed using a polynucleotide that encodes a first polypeptide and at least a second polypeptide. As such, the polynucleotide can encode, for example, a first polypeptide and a second polypeptide; a first polypeptide, a second polypeptide, and a third polypeptide; etc. Furthermore, any or all of the encoded polypeptides can be the same or different. The polypeptides expressed in chloroplasts of the microalga C. reinhardtii may be assembled to form functional polypeptides and protein complexes. As such, a method of the invention provides a means to produce functional protein complexes, including, for example, dimers, trimers, and tetramers, wherein the subunits of the complexes can be the same or different (e.g., homodimers or heterodimers, respectively).

The term “recombinant nucleic acid molecule” is used herein to refer to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked and, for example, can encode a fusion polypeptide, or can comprise an encoding nucleotide sequence and a regulatory element. A recombinant nucleic acid molecule also can be based on, but manipulated so as to be different, from a naturally occurring polynucleotide, (e.g. biased for chloroplast codon usage, insertion of a restriction enzyme site, insertion of a promoter, insertion of an origin of replication). A recombinant nucleic acid molecule may further contain a peptide tag (e.g., His-6 tag), which can facilitate identification of expression of the polypeptide in a cell. Additional tags include, for example: a FLAG epitope, a c-myc epitope; biotin; and glutathione S-transferase. Such tags can be detected by any method known in the art (e.g., anti-tag antibodies, streptavidin). Such tags may also be used to isolate the operatively linked polypeptide(s), for example by affinity chromatography.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995). Polynucleotides useful for practicing a method of the present invention may be isolated from any organism.

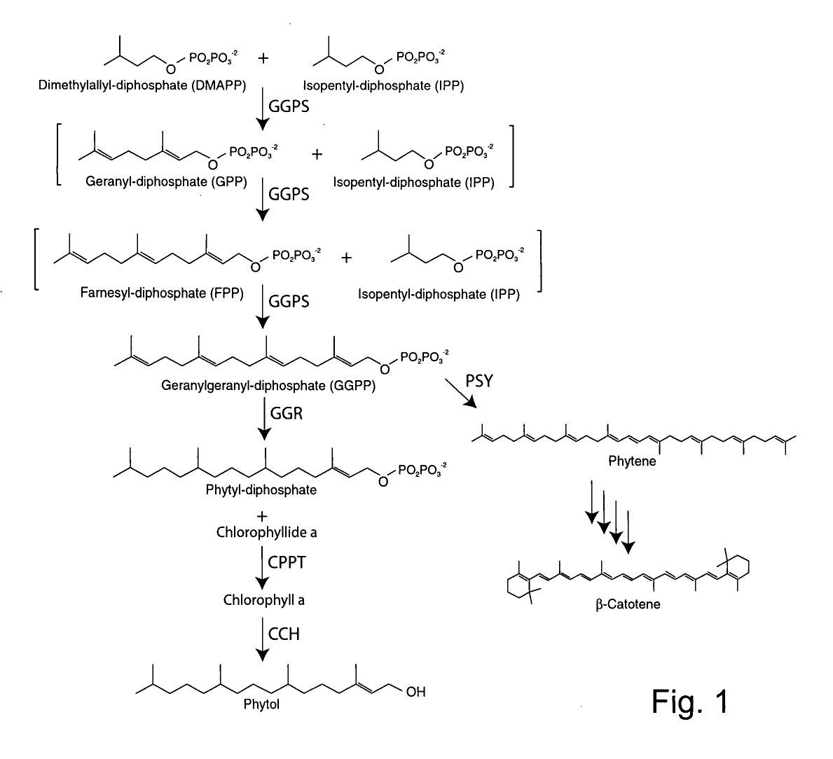

The invention may take advantage of naturally occurring product production pathways in an NVPO. An example of such a pathway (for the production of phytol and β-carotene) is shown in FIG. 1. One of skill in the art will recognize that this isoprenoid production pathway is provided merely by way of example to further illustrate one embodiment.

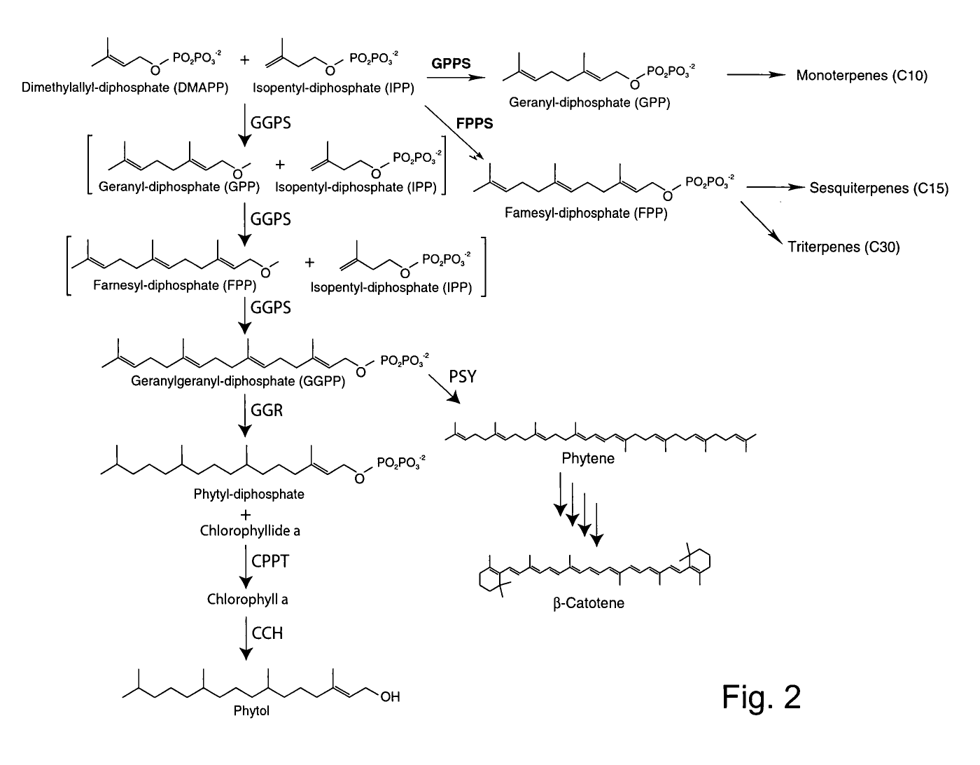

One aspect of the present invention is to modify the phytol/β-carotene pathway to produce non-naturally occurring products and/or increase the production of naturally occurring products. FIG. 2 illustrates one potential for modification of the pathway illustrated in FIG. 1. By inserting an exogenous geranyl-diphosphate synthase (GPPS) and/or farsenyl-diphosphate synthase (FPPS), the production of GPP and FPP can be increased. For example, a host organism (e.g., C. reinhardtii, D. salina) can be transformed with any of the sequences encoding GPP or FPP synthases listed in Tables 5 or 7 (e.g., SEQ ID NOs. 82, 87-94, 118, and/or 180-191). Furthermore, as exemplified in the examples below, introduction of a GPPS or FPPS may be accompanied by the insertion of an exogenous gene encoding an enzyme (e.g., limonene synthase, zingiberene synthase, chlorophyllohydrolase) which leads to the production of isoprenoids of interest (e.g., monoterpenes, sesquiterpenes, and triterpenes) which are not naturally produced by the NVPO. A non-limiting list of enzymes which may be used to transform NVPOs—alone, or in combination—is provided in Tables 5-8.

Insertion of genes encoding enzymes of the present invention may lead to increased production of a naturally occurring isoprenoid (e.g., GPP, FPP, phytol, phytoene, β-carotene). For example, production of naturally occurring isoprenoids (e.g., GPP, FPP, phytoene) may be increased by: 1) introducing extra copies of an endogenous or exogenous gene encoding a synthetic enzyme which produces the isoprenoid; 2) introducing a regulatory element (e.g., constitutive promoter, inducible promoter) to control expression of a naturally occurring synthetic enzyme; and/or 3) introduction of an exogenous nucleic acid which increases production of a naturally occurring isoprenoid through an indirect route (e.g., an exogenous GPPS may increase the intracellular concentration of GPP, providing more substrate for a phytol/chlorophyll synthesis pathway).

Thus, production of certain naturally occurring isoprenoids may be increased. For purposes of illustration only, the isoprenoid, phytol, is naturally produced by a number of NVPOs, including C. reinhardtii. Generally, the amount of phytol in wild type strains of C. reinhardtii is less than 1% by weight. The present disclosure provides for several mechanisms which may increase production of phytol. In one example, a regulatory element which drives constitutive or inducible expression of an endogenous gene (e.g., GPP synthase) may be introduced into a genome of the organism to express the gene at a higher level than that which is achieved by the naturally occurring regulatory elements. Alternately, one or more exogenous isoprenoid synthases may be introduced into a genome of the organism. Such synthases may be homologous or non-homologous to the target NVPO (e.g. a GPP synthase from a related organism, an FPP synthase). Alternately, exogenous enzymes (e.g., phosphatases, pyrophosphatases) may be introduced into the target NVPO. Such enzymes may act on naturally occurring substrates (e.g., GGPP, phytyl-diphosphate) or may act on substrates produced by other exogenous genes introduced into the host NVPO. In some instances, exogenous enzymes may produce the isoprenoid of interest (e.g., phytol) or may produce a precursor for an enzyme which then acts to produce the isoprenoid of interest. In still another approach, an enzyme may be introduced or upregulated which causes the degradation of a product produced by the host NVPO—either naturally or as the result of an introduced gene—thereby producing the isoprenoid of interest. For example, a chlorophyllidohydrolase may be introduced into the host cell to promote degradation of chlorophyll into phytol.

Utilizing such approaches, a modified NVPO may comprise about 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, or more. Where desired, phytol can be collected from modified NVPOs and concentrated to about 5%, 10%, 15%, 20%, 25%, 30%, 35%, 40%, 45%, 50%, 55%, 60%, 65%, 70%, 76%, 80%, 85%, 90%, 95%, or higher. In some instances, compositions comprising phytol collected from an NVPO of the present invention may also comprise portions of the cells of the NVPO (e.g., cell wall material, cell membrane material, proteins, carbohydrates, nucleic acids, etc.).

Pathways utilized for the present invention may involve enzymes present in the cytosol, in a plastid (e.g., chloroplast), or both. Exogenous nucleic acids encoding the enzymes of embodiments of the invention may be introduced into a host cell, such that the enzyme encoded is active in the cytosol or in a plastid, or both. In some embodiments, a naturally occurring enzyme which is present in one intracellular compartment (e.g., in the cytosol) may be expressed in a different intracellular locale (e.g., in the chloroplast), or in both the naturally occurring and non-naturally occurring locales following transformation of the host cell.

To illustrate this concept, and merely by way of example, a non-vascular photosynthetic microalga species can be genetically engineered to produce an isoprenoid, such as limonene (a molecule of high value in the specialty chemical and petrochemical industries). Limonene is a monoterpene that is a pure hydrocarbon, only composed of hydrogen and carbon atoms. Limonene is not naturally produced in the species, Chlamydomonas rheinhardii. Production of limonene in these microalgae can be achieved by engineering the microalgae to express the heterologous enzyme limonene synthase in the chloroplast. Limonene synthase can convert the terpene precursor geranyl pyrophosphate into limonene. Unlike limonene, geranyl pyrophosphate is naturally present in the chloroplast of microalgae. The expression of the limonene synthase can be accomplished by inserting the heterologous gene encoding limonene synthase into the chloroplast genome of the microalgae. The modified strain of microalgae is then made homoplasmic to ensure that the limonene gene will be stably maintained in the chloroplast genome of all descendents. A microalga is homoplasmic for a gene when the inserted gene is present in all copies of the chloroplast genome. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein.

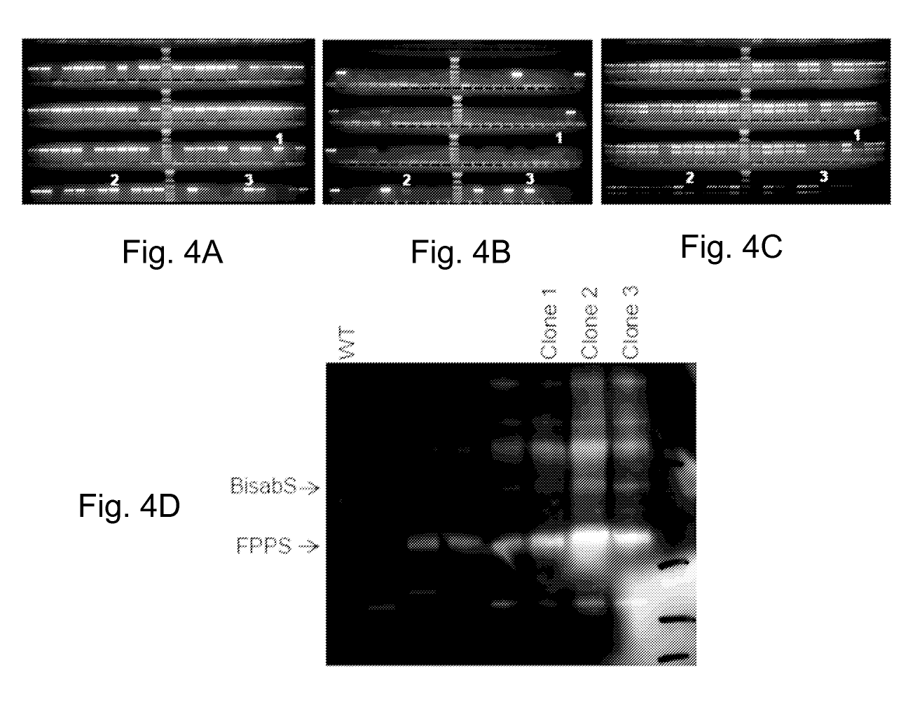

Briefly, the process of determining plasmic state of an organism of the present invention involves screening transformants for the presence of exogenous nucleic acids and the absence of wild-type nucleic acids at a given locus of interest. Such approaches are utilized in Examples 1 and 2 below (FIGS. 4A-C and 6A-C).

Any of the vectors herein (e.g. including any of the expression vectors described above) can comprise a nucleotide sequence(s) encoding a protein or polypeptide that allows or improves secretion of a product produced by a transformed organism. The protein or polypeptide molecule affects, increases, upregulates, or modulates the secretion of a product from a host cell or organism.

An expression vector may comprise a sequence encoding a protein or polypeptide that allows or improves secretion of a product molecule and a nucleotide sequence encoding a synthase from an isoprenoid pathway.

Selectable Markers.

In some instances, a heterologous sequence in an expression vector encodes a dominant selectable marker for selection of the transformed organisms. Thus, organisms that have been subjected to transformation can be cultured in the presence of a compound (e.g., to which a dominant selectable marker confers resistance) that allows for the dominant selection of transformed host cells. Examples of compounds to which selectable markers confer resistance when expressed in the host organism include metabolic inhibitors (i.e., compounds that inhibit algal metabolism), such as antibiotics, fungicides, algicides, bactericides, and herbicides. Functionally, such compounds may be toxic to the cell or otherwise inhibit metabolism by functioning as protein or nucleic acid binding agents. For example, such compounds can inhibit translation, transcription, enzyme function, cell growth, cell division and/or microtubule formation. Dominant selectable markers suitable for use in the present invention can be selected from any known or subsequently identified selectable markers, including markers derived from fungal and bacterial sources (see e.g. U.S. Pat. No. 5,661,017). In other embodiments, wild-type homologous genes that complement auxotrophic mutant strains may also be used as selectable marker systems, for example in some green algae (see e.g. Kindle et al., J. Cell Biol., 109:2589-2601 (1989) which discusses the transformation of a nitrate reductase deficient mutant of Chlamydomonas reinhardtii with a gene encoding nitrate reductase).

Regulatory Control Sequences.

Any of the expression vectors herein can further comprise a regulatory control sequence. A regulatory control sequence may include for example, promoter(s), operator(s), repressor(s), enhancer(s), transcription termination sequence(s), sequence(s) that regulate translation, or other regulatory control sequence(s) that are compatible with the host cell and control the expression of the nucleic acid molecules of the present invention. In some cases, a regulatory control sequence includes transcription control sequence(s) that are able to control, modulate, or effect the initiation, elongation, and/or termination of transcription. For example, a regulatory control sequence can increase transcription and translation rate and/or efficiency of a gene or gene product in an organism, wherein expression of the gene or gene product is upregulated resulting (directly or indirectly) in the increased production, secretion, or both, of a product described herein. The regulatory control sequence may also result in the increase of production, secretion, or both, of a product by increasing the stability of a gene or gene product.

A regulatory control sequence can be autologous or heterologous, and if heterologous, may be homologous. The regulatory control sequence may encode one or more polypeptides which are enzymes that promote expression and production of products. For example, a heterologous regulatory control sequence may be derived from another species of the same genus of the organism (e.g., another algal species) and encode a synthase in an algae. In another example, an autologous regulatory control sequence can be derived from an organism in which an expression vector is to be expressed.

Depending on the application, regulatory control sequences can be used that effect inducible or constitutive expression. The algal regulatory control sequences can be used, and can be of nuclear, viral, extrachromosomal, mitochondrial, or chloroplastic origin.

Suitable regulatory control sequences include those naturally associated with the nucleotide sequence to be expressed (for example, an algal promoter operably linked with an algal-derived nucleotide sequence in nature). Suitable regulatory control sequences include regulatory control sequences not naturally associated with the nucleic acid molecule to be expressed (for example, an algal promoter of one species operatively linked to an nucleotide sequence of another organism or algal species). The latter regulatory control sequences can be a sequence that controls expression of another gene within the same species (i.e., autologous) or can be derived from a different organism or species (i.e., heterologous).

To determine whether a putative regulatory control sequence is suitable, the putative regulatory control sequence is linked to a nucleic acid molecule typically encodes a protein that produces an easily detectable signal. The construction may then be introduced into an alga or other organism by standard techniques and expression thereof is monitored. For example, if the nucleic acid molecule encodes a dominant selectable marker, the alga or organism to be used is tested for the ability to grow in the presence of a compound for which the marker provides resistance.

In some cases, a regulatory control sequence is a promoter, such as a promoter adapted for expression of a nucleotide sequence in a non-vascular, photosynthetic organism. For example, the promoter may be an algal promoter, for example as described in U.S. Publ. Appl. Nos. 2006/0234368 and 2004/0014174, and in Hallmann, Transgenic Plant J. 1:81-98 (2007). The promoter may be a chloroplast specific promoter or a nuclear promoter. The promoter may an EF1-α gene promoter or a D promoter. In some embodiments, the synthase is operably linked to the EF1-α gene promoter. In other embodiments, the synthase is operably linked to the D promoter.

A regulatory control sequences herein can be found in a variety of locations, including for example, coding and non-coding regions, 5′ untranslated regions (e.g., regions upstream from the coding region), and 3′ untranslated regions (e.g., regions downstream from the coding region). Thus, in some instances an autologous or heterologous nucleotide sequence can include one or more 3′ or 5′ untranslated regions, one or more introns, or one or more exons.

For example, in some embodiments, a regulatory control sequence can comprise a Cyclotella cryptica acetyl-CoA carboxylase 5′ untranslated regulatory control sequence or a Cyclotella cryptica acetyl-CoA carboxylase 3′-untranslated regulatory control sequence (U.S. Pat. No. 5,661,017).

A regulatory control sequence may also encode chimeric or fusion polypeptides, such as protein AB, or SAA, that promotes expression of heterologous nucleotide sequences and proteins. Other regulatory control sequences include autologous intron sequences that may promote translation of a heterologous sequence.

The regulatory control sequences used in any of the expression vectors herein may be inducible. Inducible regulatory control sequences, such as promoters, can be inducible by light, for example. Regulatory control sequences may also be autoregulatable. Examples of autoregulatable regulatory control sequences include those that are autoregulated by, for example, endogenous ATP levels or by the product produced by the organism. In some instances, the regulatory control sequences may be inducible by an exogenous agent. Other inducible elements are well known in the art and may be adapted for use in the present invention.

Various combinations of the regulatory control sequences described herein may be embodied by the present invention and combined with other features of the present invention. In some cases, an expression vector comprises one or more regulatory control sequences operatively linked to a nucleotide sequence encoding a polypeptide. Such sequences may, for example, upregulate secretion, production, or both, of a product described herein. In some cases, an expression vector comprises one or more regulatory control sequences operatively linked to a nucleotide sequence encoding a polypeptide that effects, for example, upregulates secretion, production, or both, of a product.

Expression.

Chloroplasts are a productive organelle of photosynthetic organisms and a site of large of amounts of protein synthesis. Any of the expression vectors herein may be selectively adapted for chloroplast expression. A number of chloroplast promoters from higher plants have been described in Kung and Lin, Nucleic Acids Res. 13: 7543-7549 (1985). Gene products may be expressed from the expression vector in the chloroplast. Gene products encoded by expression vectors may also be targeted to the chloroplast by chloroplast targeting sequences. For example, targeting an expression vector or the gene product(s) encoded by an expression vector to the chloroplast may further enhance the effects provided by the regulatory control sequences and sequence(s) encoding a protein or peptide that allows or improves secretion of a fuel molecule.

Various combinations of the chloroplast targeting described herein may be embodied by the present invention and combined with other features of the present invention. For example, a nucleotide sequence encoding a terpene synthase may be operably linked to a nucleotide sequence encoding a chloroplast targeting sequence. A host cell may be transformed with an expression vector encoding limonene synthase targeted to the chloroplast, and thus, may produce more limonene synthase as compared to a host cell transformed with an expression vector encoding limonene synthase but not a chloroplast targeting sequence. The increased limonene synthase expression may produce more of the limonene in comparison to the host cell that produces less. Tables 5 and 7 provide examples of nucleic acids encoding isoprenoid producing enzymes useful in the present invention. Tables 6 and 8 provide these nucleic acid sequences with the addition of restriction enzyme sites. The sequences in Tables 5-8 are also codon-biased for expression in C. reinhardtii. Such sites, as will be readily apparent, can be used to integrate the nucleic acids into a vector.

In yet another example, an expression vector comprising a nucleotide sequence encoding an enzyme that produces a product (e.g. fuel product, fragrance product, insecticide product) not naturally produced by the organism by using precursors that are naturally produced by the organism as substrates, is targeted to the chloroplast. By targeting the enzyme to the chloroplast, production of the product may be increased in comparison to a host cell wherein the enzyme is expressed, but not targeted to the chloroplast. Without being bound by theory, this may be due to increased precursors being produced in the chloroplast and thus, more product may be produced by the enzyme encoded by the introduced nucleotide sequence.

Products.

Examples of products contemplated herein include hydrocarbon products and hydrocarbon derivative products. A hydrocarbon product is one that consists of only hydrogen molecules and carbon molecules. A hydrocarbon derivative product is a hydrocarbon product with one or more heteroatoms, wherein the heteroatom is any atom that is not hydrogen or carbon. Examples of heteroatoms include, but not limited to, nitrogen, oxygen, sulfur, and phosphorus. Some products are hydrocarbon-rich, wherein as least 50%, 60%, 70%, 80%, 90%, or 95% of the product by weight is made up carbon and hydrogen.

Examples of hydrocarbon and hydrocarbon derivative products that can be produced using the compositions and methods herein include terpenes, and their derivatives, terpenoids. A terpene is a molecule made of isoprene (C5) units and is not necessarily a pure a hydrocarbon. Terpenes are typically derived from isoprene units. Isoprene units are five-carbon units (C5). Terpenes are hydrocarbons that can be modified (e.g. oxidized, methyl groups removed, etc.) or its carbon skeleton rearranged, to form derivatives of terpenes, such as isoprenoids.

Isoprenoids (also known as terpenoids) are derived from isoprene subunits but are modified, such as by the addition of heteroatoms such as oxygen, by carbon skeleton rearrangement, and by alkylation. Isoprenoids generally have a number of carbon atoms which is evenly divisible by five, but this is not a requirement as “irregular” terpenoids are known. Carotenoids, such as carotenes and xanthophylls, are an example of a isoprenoid as a useful product. A steroid is another example of a terpenoid. Examples of isoprenoids include, but are not limited to, hemiterpenes (C5), monoterpenes (C10), sesquiterpenes (C15), diterpenes (C20), triterpenes (C30), tetraterpenes (C40), and polyterpenes (Cₙ, wherein “n” is equal to or greater than 45). Other examples of isoprenoids include, but are not limited to, limonene, 1,8-cineole, α-pinene, camphene, (+)-sabinene, myrcene, abietadiene, taxadiene, farnesyl pyrophosphate, amorphadiene, (E)-α-bisabolene, zingiberene, or diapophytoene, and their derivatives.

Isoprenoid precursors are thought to be generated by two pathways. The mevalonate pathway, or HMG-CoA reductase pathway, generates dimethylallyl pyrophosphate (DMAPP) and isopentyl pyrophosphate (IPP), the common C5 precursor for isoprenoids. The non-mevalonate pathway is an alternative pathway to form DMAPP and IPP. The DMAPP and IPP may be condensed to form geranyl-diphosphate (GPP), or other precursors, such as farnesyl-diphosphate (FPP), geranylgeranyl-diphosphate (GGPP), from which higher isoprenes are formed.

Examples of products which can include the isoprenoids of the present invention include, but are not limited to, fuel products, fragrance products, and insecticide products. In some instances, a product may be used directly. In other instances, the product may be used as a “feedstock” to produce another product. For example, where the product is an isoprenoid, the isoprenoid may be hydrogenated and “cracked” to produce a shorter chain hydrocarbon (e.g., farnesene is hydrogenated to produce farnesane which is then cracked to produce propane, butane, octane, or other fuel products).

The products produced by the present invention may be naturally, or non-naturally (e.g., as a result of transformation) produced by the host cell(s) and/or organism(s) transformed. The product may also be a novel molecule not present in nature. For example, products naturally produced in algae may be terpenes such as carotenoids (e.g. beta-carotene). Examples of products not naturally produced by algae may include a non-native terpene such as limonene. The host cell may be genetically modified, for example by transformation with a sequence to encourage the secretion of limonene.

Fuel Products

Examples of fuel products include petrochemical products and their precursors and all other substances that may be useful in the petrochemical industry. Fuel products include, for example, petroleum products, precursors of petroleum, as well as petrochemicals and precursors thereof. The fuel or fuel products may be used in a combustor such as a boiler, kiln, dryer or furnace. Other examples of combustors are internal combustion engines such as vehicle engines or generators, including gasoline engines, diesel engines, jet engines, and others. Fuel products may also be used to produce plastics, resins, fibers, elastomers, lubricants, and gels.

Fuel products can include small alkanes (for example, 1 to approximately 4 carbons) such as methane, ethane, propane, or butane, which may be used for heating (such as in cooking) or making plastics. Fuel products may also include molecules with a carbon backbone of approximately 5 to approximately 9 carbon atoms, such as naptha or ligroin, or their precursors. Other fuel products may be about 5 to about 12 carbon atoms or cycloalkanes used as gasoline or motor fuel. Molecules and aromatics of approximately 10 to approximately 18 carbons, such as kerosene, or its precursors, may also be fuel products. Fuel products may also include molecules, or their precursors, with more than 12 carbons, such as used for lubricating oil. Other fuel products include heavy gas or fuel oil, or their precursors, typically containing alkanes, cycloalkanes, and aromatics of approximately 20 to approximately 70 carbons. Fuel products also includes other residuals that can be derived from or found in crude oil, such as coke, asphalt, tar, and waxes, generally containing multiple rings with about 70 or more carbons, and their precursors.

The various fuel products may be further refined to a final product for an end user by a number of processes. Refining can occur by fractional distillation. For example, a mixture of fuel products, such as a mix of different hydrocarbons with different various chain lengths may be separated into various components by fractional distillation.

Refining may also include any one or more of the following steps; cracking, unifying, or altering the fuel product. Large fuel products, such as large hydrocarbons (e.g. ≧C10), may be broken down into smaller fragments by cracking. Cracking may be performed by heat or high pressure, such as by steam, visbreaking, or coking. Fuel products may also be refined by visbreaking, for example reducing the viscosity of heavy oils. Refining may also include coking, wherein a heavy, almost pure carbon residue is produced. Cracking may also be performed by catalytic means to enhance the rate of the cracking reaction by using catalysts such as, but not limited to, zeolite, aluminum hydrosilicate, bauxite, or silica-alumina. Catalysis may be by fluid catalytic cracking, whereby a hot catalyst, such as zeolite, is used to catalyze cracking reactions. Catalysis may also be performed by hydrocracking, where lower temperatures are generally used in comparison to fluid catalytic cracking. Hydrocracking typically occurs in the presence of elevated partial pressure of hydrogen gas. Fuel products may be refined by catalytic cracking to generate diesel, gasoline, and/or kerosene.

The fuel products may also be refined by combining them in a unification step, for example by using catalysts, such as platinum or a platinum-rhenium mix. The unification process typically produces hydrogen gas, a by-product which may be used in cracking.

The fuel products may also be refined by altering or rearranging or restructuring hydrocarbons into smaller molecules. There are a number of chemical reactions that occur in the catalytic reforming process of which are known to one of ordinary skill in the arts. Generally, catalytic reforming is performed in the presence of a catalyst and high partial pressure of hydrogen. One common process is alkylation. For example, propylene and butylene are mixed with a catalyst such as hydrofluoric acid or sulfuric acid.

The fuel products may also be blended or combined into mixtures to obtain an end product. For example, the fuel products may be blended to form gasoline of various grades, gasoline with or without additives, lubricating oils of various weights and grades, kerosene of various grades, jet fuel, diesel fuel, heating oil, and chemicals for making plastics and other polymers. Compositions of the fuel products described herein may be combined or blended with fuel products produced by other means.

Some fuel products produced from the host cells of the invention, especially after refining, will be identical to existing petrochemicals, i.e. same structure. Some of the fuel products may not be the same as existing petrochemicals. However, although a molecule may not exist in conventional petrochemicals or refining, it may still be useful in these industries. For example, a hydrocarbon could be produced that is in the boiling point range of gasoline, and that could be used as gasoline or an additive, even though it does not normally occur in gasoline.

Methods.

Thus, a product (e.g. isoprenoid, fuel product, fragrance product, insecticide product) may be produced by a method that comprises: transforming a host organism (e.g., non-vascular, photosynthetic organism) with an expression vector; growing the organism; and collecting the product produced by the organism. In a related yet distinct aspect, the present invention provides a method for producing a product comprising: transforming a photosynthetic organism with an expression vector, growing the organism; and collecting the product produced by the oganism. The expression vector is typically the type of expression vector described herein, and is specifically used to add additional biosynthetic capacity to an organism or to modify an existing biosynthetic pathway within the organisms, either with the intension of increasing or allowing the production of a molecule by the photosynthetic organism.

The methods herein comprise selecting genes that are useful to produce products, such as isoprenoids, fuels, fragrances, and insecticides, transforming a cell of a photosynthetic organism with such gene(s), and growing such organisms under conditions suitable to allow the product to be produced. Organisms of the present invention can be cultured in conventional fermentation bioreactors, which include, but are not limited to, batch, fed-batch, cell recycle, and continuous fermentors. Further, they may be grown in photobioreactors (see e.g. US Appl. Publ. No. 20050260553; U.S. Pat. No. 5,958,761; U.S. Pat. No. 6,083,740). Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

A host organism is an organism comprising a host cell. In preferred embodiments, the host organism is photosynthetic. A photosynthetic organism is one that naturally photosynthesizes (has a plastid) or that is genetically engineered or otherwise modified to be photosynthetic. In some instances, a photosynthetic organism may be transformed with a construct of the invention which renders all or part of the photosynthetic apparatus inoperable. In some instances a host organism is non-vascular and photosynthetic. The host cell can be prokaryotic. Examples of some prokaryotic organisms of the present invention include, but are not limited to, cyanobacteria (e.g., Synechococcus, Synechocystis, Athrospira). The host organism can be unicellular or multicellular. In most embodiments, the host organism is eukaryotic (e.g. green algae, red algae, brown algae). In preferred embodiments, the host cell is a microalga (e.g., Chlamydomonas reinhardtii, Dunaliella salina, Haematococcus pluvalis, Scenedesmus dimorphus, D. viridis, or D. tertiolecta). Examples of organisms contemplated herein include, but are not limited to, rhodophyta, chlorophyta, heterokontophyta, tribophyta, glaucophyta, chlorarachniophytes, euglenoids, haptophyta, cryptomonads, dinoflagellata, and phytoplankton.

Some of the host organisms which may be used to practice the present invention are halophilic (e.g., Dunaliella salina, D. viridis, or D. tertiolecta). For example, D. salina can grow in ocean water and salt lakes (salinity from 30-300 parts per thousand) and high salinity media (e.g., artificial seawater medium, seawater nutrient agar, brackish water medium, seawater medium, etc.). In some embodiments of the invention, a host cell comprising a vector of the present invention can be grown in a liquid environment which is 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, etc.) may also be present in the liquid environments.

Where a halophilic organism is utilized for the present invention, it may be transformed with any of the vectors described herein. For example, D. salina may be transformed with a vector which is capable of insertion into the chloroplast genome and which contains nucleic acids which encode an isoprenoid producing enzyme (e.g., FPP synthase, zingiberene synthase, squalene synthase). Transformed halophilic organisms may then be grown in high-saline environments (e.g., salt lakes, salt ponds, high-saline media, etc.) to produce the products (e.g., isoprenoids) of interest. Isolation of the products may involve removing a transformed organism from a high-saline environment prior to extracting the product from the organism. In instances where the product is secreted into the surrounding environment, it may be necessary to desalinate the liquid environment prior to any further processing of the product.

A host organism may be grown under conditions which permit photosynthesis, however, this is not a requirement (e.g., a host organism may be grown in the absence of light). In some instances, the host organism may be genetically modified in such a way that photosynthetic capability is diminished and/or destroyed (see examples below). In growth conditions where a host organism is not capable of photosynthesis (e.g., because of the absence of light and/or genetic modification), typically, the organism will be provided with the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, or an organism-specific requirement. Organic carbon sources include any source of carbon which the host organism is able to metabolize including, but not limited to, acetate, simple carbohydrates (e.g., glucose, sucrose, lactose), complex carbohydrates (e.g., starch, glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

A host organism may also be grown on land, e.g., landfills. In some cases, host organism(s) are grown near ethanol production plants or other facilities or regions (e.g., cities, highways, etc.) generating CO₂. As such, the methods herein contemplate business methods for selling carbon credits to ethanol plants or other facilities or regions generating CO₂ while making fuels by growing one or more of the modified organisms described herein near the ethanol production plant.

Further, the organisms may be grown in outdoor open water, such as ponds, the ocean, sea, rivers, waterbeds, marsh water, shallow pools, lakes, reservoirs, etc. When grown in water, the organisms can be contained in a halo like object comprising of lego-like particles. The halo object encircles the algae and allows it to retain nutrients from the water beneath while keeping it in open sunlight.

In some instances, organisms can be grown in containers wherein each container comprises 1 or 2 or a plurality of organisms. The containers can be configured to float on water. For example, a container can be filled by a combination of air and water to make the container and the host organism(s) in it buoyant. A host organism that is adapted to grow in fresh water can thus be grown in salt water (i.e., the ocean) and vice versa. This mechanism allows for automatic death of the organism if there is any damage to the container.

In some instances a plurality of containers can be contained within a halo-like structure as described above. For example, up to 100, 1,000, 10,000, 100,000, or 1,000,000 containers can be arranged in a meter-square of a halo-like structure.

In some embodiments, the product (e.g. fuel product, fragrance product, insecticide product) is collected by harvesting the organism. The product may then be extracted from the organism. In some instances, the product may be produced without killing the organisms. Producing and/or expressing the product may not render the organism unviable.

In some embodiments, the production of the product (e.g. fuel product, fragrance product, insecticide product) is inducible. The product may be induced to be expressed and/or produced, for example, by exposure to light. In yet other embodiments, the production of the product is autoregulatable. The product may form a feedback loop, wherein when the product (e.g. fuel product, fragrance product, insecticide product) reaches a certain level, expression or secretion of the product may be inhibited. In other embodiments, the level of a metabolite of the organism inhibits expression or secretion of the product. For example, endogenous ATP produced by the organism as a result of increased energy production to express or produce the product, may form a feedback loop to inhibit expression of the product. In yet another embodiment, production of the product may be inducible, for example, by light or an exogenous agent. For example, an expression vector for effecting production of a product in the host organism may comprise an inducible regulatory control sequence that is activated or inactivated by an exogenous agent.

The present invention also relates to methods for screening for new genes/expression vectors to create any of the fuel products described herein. Such methods comprise the steps of: (1) inserting a candidate expression vector of nucleic acids into a photosynthetic organism, (2) collecting a putative fuel product produced there from, (3) applying the putative fuel product to a mass spectrometer to determine a characteristic of the putative fuel product, and whether it may be used as a fuel product. In some embodiments, step (2) may comprise collecting a known fuel product and whether a candidate expression vector increases production or secretion of the fuel product relative to a photosynthetic organism without the candidate expression vector.

Other Methods

The present invention also provides a business method comprising providing a carbon credit to a party growing a genetically modified non-vascular, photosynthetic organism adapted to produce a fuel product. The method of producing a fuel product provided by the present invention provides a possibly more environmentally friendly way of generating fuel products relative to current methods. As such, the methods and compositions described herein may be used in a business method in exchange for carbon credits.

Carbon credits may be an allowance, permit, credit, or the like which are or have been allowed, authorized, or recognized by some relevant sovereign entity (such as but not limited to a city (including municipalities of all sizes and types including both incorporated and unincorporated municipalities), a county, a state or province, or a nation, as well as related governmental entities such regional, multi-national, or other international bodies such as the United Nations or the European Union).

The carbon credit may be substantially received directly from a regulatory agency or administrative entity. In other instances, they may be received indirectly, for example, an entity using the methods or compositions herein may receive the carbon credits directly from a regulatory agency, and may then transfer the carbon credits to another entity. Transfer of the carbon credit may be in association with a given process, product using the genetically modified non-vascular, photosynthetic organism adapted to produce a fuel product.

For example, a first entity may be identified that provides a consumable product that is distributed for consumption in an end-user mobile platform, wherein the consumption and/or production of the consumable product includes a corresponding resultant emission. For example, combustion of diesel fuel often results in the environmental release of corresponding nitrogen oxides combustion of diesel fuel often results in the environmental release of corresponding nitrogen oxides and combustion of gasoline often results in the environmental release of corresponding sulfur oxide.

The first party may adopt a method of producing its products using the genetically modified organisms described above, or use the products generated by the genetically modified organisms described above in their compositions, resulting in less harmful effects on the environment than conventional methods of generating, for example, diesel fuel. Thus off-setting the environmental effects of the end product. The first party may then receive a carbon, or emission, credit as a result of a reduction of the total emission. The carbon credit may be received from a regulatory or administrative agency, or may be transferred to the first party from a second party, wherein the second party may have sold the genetically modified organism or the products of the genetically modified organism to the first party.

The carbon credit may be exchanged for a substantially liquid monetary instrument. For example, the carbon credit may be exchanged for a cash equivalent, such as cash, check, and the like. The carbon credit may also be exchanged for a legal grant regarding an intellectual property right, for example, but not limited to, an assignment or a license. The carbon credit may also be exchanged for a government tax subsidy or access to purchasers of a given market. The carbon credit may also be exchanged for use of another carbon emission process, such as one not comprising growing the organism. For example, a party may have a limited number of emissions it may release in a time period, for example, a month or a year, and going over the limit may incur fines and penalties. However, with carbon credits, the party going over the limit may exchange of carbon credits to offset the fines or penalties or may be taken into account when determining the amount of emissions generated by the party.

The business methods of the invention can also involve the production of products other than fuel products, such as fragrances and insecticides. Business methods associated with fuel products, including those involving the use of carbon credits, are also relevant to the production of other types of useful products and materials.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The following examples merely illustrate the invention disclosed herein, but do not limit it.

### EXAMPLES

### Example 1

### Production of FPP Synthases and Sesquiterpene Synthases in C. reinhardtii

In this example a nucleic acids encoding FPP synthase from G. gallus and bisabolene synthase from P. abies were introduced into C. reinhardtii. Transforming DNA is shown graphically in FIG. 3. For these examples, the transforming DNA was contained in a vector with E. coli elements (e.g., origin of replication, antibiotic resistance marker). In this instance the gene encoding FPP synthase (SEQ ID NO. 82, Table 5; SEQ ID NO. 135, Table 6) is the segment labeled “transgene” in FIG. 3 and is regulated by the 5′ UTR and promoter sequence for the psbA gene from C. reinhardtii and the 3′ UTR for the psbA gene from C. reinhardtii, and the segment labeled “Selection Marker” is the kanamycin resistance encoding gene from bacteria, which is regulated by the 5′ UTR and promoter sequence for the atpA gene from C. reinhardtii and the 3′ UTR sequence for the rbcL gene from C. reinhardtii. The bisabolene synthase gene (SEQ ID NO. 115, Table 5; SEQ ID NO. 168, Table 6) is the segment labeled “transgene” in FIG. 3 and is regulated by the 5′ UTR and promoter sequence for the psbA gene from C. reinhardtii and the 3′ UTR for the psbA gene from C. reinhardtii, and the segment labeled “Selection Marker” is the streptomycin resistance encoding gene from bacteria, which is regulated by the 5′ UTR and promoter sequence for the atpA gene from C. reinhardtii and the 3′ UTR sequence for the rbcL gene from C. reinhardtii. The FPP synthase transgene cassette is targeted to the psbA loci of C. reinhardtii via the segments labeled “Homology A” and “Homology B,” which are identical to sequences of DNA flanking the psbA loci on the 5′ and 3′ sides, respectively. The bisabolene synthase transgene cassette is targeted to the 3HB locus of C. reinhardtii via the segments labeled “Homology C” and “Homology D,” which are identical to sequences of DNA flanking the 3HB locus on the 5′ and 3′ sides, respectively. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain 137c (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells resuspended in 4 ml TAP medium for subsequent chloroplast transformation by particle bombardment (Cohen et al., supra, 1998). All transformations were carried out under kanamycin selection (100 μg/ml) in which resistance was conferred by the gene encoded by the segment in FIG. 3 labeled “Selection Marker.” (Chlamydomonas Stock Center, Duke University).

PCR was used to identify transformed strains. For PCR analysis, 106 algae cells (from agar plate or liquid culture) were suspended in 10 mM EDTA and heated to 95° C. for 10 minutes, then cooled to near 23° C. A PCR cocktail consisting of reaction buffer, MgCl2, dNTPs, PCR primer pair(s) (Table 4), DNA polymerase, and water was prepared. Algae lysate in EDTA was added to provide template for reaction. Magnesium concentration was varied to compensate for amount and concentration of algae lysate in EDTA that was added. Annealing temperature gradients were employed to determine optimal annealing temperature for specific primer pairs.

To identify strains that contain the FPP synthase gene, a primer pair was used in which one primer anneals to a site within the psbA 5′UTR (SEQ ID NO. 55) and the other primer (SEQ ID NO. 66) anneals within the FPP synthase coding segment. Desired clones are those that yield a PCR product of expected size. To identify strains that contain the bisabolene synthase gene, a primer pair was used in which one primer anneals to a site within the psbA 5′UTR (SEQ ID NO. 55) and the other primer anneals within the bisabolene synthase coding segment (SEQ ID NO. 73). Desired clones are those that yield a PCR product of expected size in both reactions.

To determine the degree to which the endogenous psbA gene locus is displaced (heteroplasmic vs. homoplasmic), a PCR reaction consisting of two sets of primer pairs were employed (in the same reaction). The first pair of primers amplifies the endogenous locus targeted by the expression vector and consists of a primer that anneals within the psbA 5′UTR (SEQ ID NO. 57) and one that anneals within the psbA coding region (SEQ ID NO. 58). The second pair of primers (SEQ ID NOs. 59 and 60) amplifies a constant, or control region that is not targeted by the expression vector, so should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used was >30 to increase sensitivity. The most desired clones are those that yield a product for the constant region but not for the endogenous gene locus. Desired clones are also those that give weak-intensity endogenous locus products relative to the control reaction. Results from this PCR are shown in FIG. 4, panels A, B, and C.

Results from this PCR on 96 clones were determined and the results are shown in FIG. 4. FIGS. 4A and 4B show PCR results using the pairs specific for the FPP synthase and squalene synthase genes, respectively. As can be seen, multiple transformed clones are positive for insertion of both the FPP synthase and squalene synthase genes (e.g. numbers 1-3). FIG. 4C shows the PCR results using the primer pairs to differentiate homoplasmic from heteroplasmic clones. As can be seen, multiple transformed clones are either homoplasmic or heteroplasmic to a degree in favor of incorporation of the transgene (e.g. numbers 1-3). Unnumbered clones demonstrate the presence of wild-type locus and, thus, were not selected for further analysis.

To determine if the FPP synthase gene led to expression of the FPP synthase and if the bisabolene synthase gene led to expression of the bisabolene synthase in transformed algae cells, both soluble proteins were immunoprecipitated and visualized by Western blot. Briefly, 500 ml of algae cell culture was harvested by centrifugation at 4000×g at 4° C. for 15 min. The supernatant was decanted and the cells resuspended in 10 ml of lysis buffer (100 mM Tris-HCl, pH=8.0, 300 mM NaCl, 2% Tween-20). Cells were lysed by sonication (10×30 sec at 35% power). Lysate was clarified by centrifugation at 14,000×g at 4° C. for 1 hour. The supernatant was removed and incubated with anti-FLAG antibody-conjugated agarose resin at 4° C. for 10 hours. Resin was separated from the lysate by gravity filtration and washed 3× with wash buffer ((100 mM Tris-HCl, pH=8.0, 300 mM NaCl, 2% Tween-20). Resin was mixed 4:1 with loading buffer (XT Sample buffer; Bio-Rad), samples were heated to 95° C. for 1 min, cooled to 23° C., and insoluble proteins were removed by centrifugation. Soluble proteins were separated by SDS-PAGE, followed by transfer to PVDF membrane. The membrane was blocked with TBST+0.5% dried, nonfat milk at 23° C. for 30 min, incubated with anti-FLAG, alkaline phosphatase-conjugate antibody (diluted 1:2,500 in TBST+0.5% dried, nonfat milk) at 4° C. for 10 hours, washed three times with TBST. Proteins were visualized with chemifluorenscent detection. Results from multiple clones (FIG. 4D) show that expression of the FPP synthase gene led to expression of the FPP synthase and expression of the bisabolene synthase gene led to expression of the bisabolene synthase.

Cultivation of C. reinhardtii transformants for expression of FPP synthase and bisabolene synthase was carried out in liquid TAP medium at 23° C. under constant illumination of 5,000 Lux on a rotary shaker set at 100 rpm, unless stated otherwise. Cultures were maintained at a density of 1×10⁷ cells per ml for at least 48 hr prior to harvest.

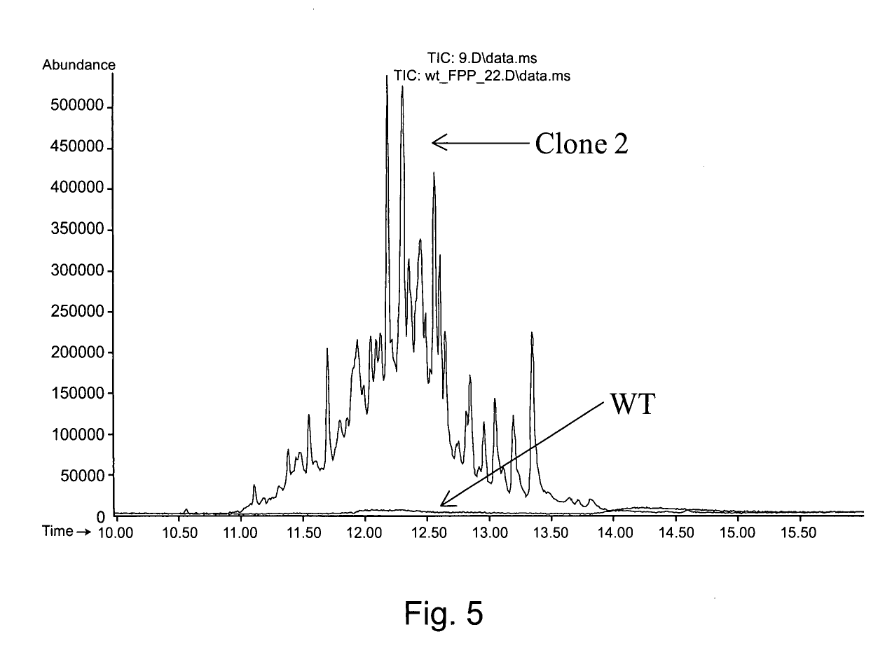

To determine whether bisabolene synthase produced in the algae chloroplast is a functional enzyme, sesquiterpene production from FPP was examined. Briefly, 50 mL of algae cell culture was harvested by centrifugation at 4000×g at 4° C. for 15 min. The supernatant was decanted and the cells resuspended in 0.5 mL of reaction buffer (25 mM HEPES, pH=7.2, 100 mM KCl, 10 mM MnCl2, 10% glycerol, and 5 mM DTT). Cells were lysed by sonication (10×30 sec at 35% power). 0.33 mg/mL of FPP were added to the lysate and the mixture was transferred to a glass vial. The reaction was overlaid with heptane and incubated at 23° C. for 12 hours. The reaction was quenched and extracted by vortexing the mixture. 0.1 mL of heptane was removed and the sample was analyzed by gas chromatography—mass spectrometry (GC-MS). Results are shown in FIG. 5. The results show a large increase (indicated by the peaks) in sesquiterpene over a wild-type strain.

### Example 2

### Production of Triterpene Molecules in C. reinhardtii

In this example a nucleic acids encoding FPP synthase from G. gallus and squalene synthase S. aureus were introduced into C. reinhardtii. Transforming DNA is shown graphically in FIG. 3. In this instance the segment labeled “Transgene 1” is the gene encoding FPP synthase (SEQ ID NO. 82, Table 5; SEQ ID NO. 135, Table 6), the segment labeled “Transgene 2” is the gene encoding squalene synthase (SEQ ID NO. 85, Table 5; SEQ ID NO. 138, Table 6), the segments labeled “5′ UTR” are the 5′ UTR and promoter sequence for the rbcL gene from C. reinhardtii, the segments labeled “3′ UTR” contain the 3′ UTR for the psbA gene from C. reinhardtii, and the segment labeled “Selection Marker” is the kanamycin resistance encoding gene from bacteria, which is regulated by the 5′ UTR and promoter sequence for the atpA gene from C. reinhardtii and the 3′ UTR sequence for the rbcL gene from C. reinhardtii. The transgene cassette is targeted to the 3HB locus of C. reinhardtii via the segments labeled “5′ Homology” and “3′ Homology,” which are identical to sequences of DNA flanking the 3HB locus on the 5′ and 3′ sides, respectively. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain 137c (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells resuspended in 4 ml TAP medium for subsequent chloroplast transformation by particle bombardment (Cohen et al., supra, 1998). All transformations were carried out under kanamycin selection (150 μg/ml) in which resistance was conferred by the gene encoded by the segment in FIG. 3 labeled “Selection Marker.” (Chlamydomonas Stock Center, Duke University).

PCR was used to identify transformed strains. For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgCl2, dNTPs, PCR primer pair(s) (Table 4), DNA polymerase, and water was prepared. Algae lysate in EDTA was added to provide template for reaction. Magnesium concentration is varied to compensate for amount and concentration of algae lysate in EDTA added. Annealing temperature gradients were employed to determine optimal annealing temperature for specific primer pairs.

To identify strains that contain the FPP synthase gene, a primer pair was used in which one primer anneals to a site within the psbC 5′UTR (SEQ ID NO. 64) and the other primer anneals within the FPP synthase coding segment (SEQ ID NO. 66). To identify strains that contain the squalene synthase gene, a primer pair was used in which one primer anneals to a site within the psbC 5′UTR (SEQ ID NO. 64) and the other primer anneals within the squalene synthase coding segment (SEQ ID NO. 72). Desired clones are those that yield a PCR product of expected size in both reactions. To determine the degree to which the endogenous gene locus is displaced (heteroplasmic vs. homoplasmic), a PCR reaction consisting of two sets of primer pairs were employed (in the same reaction). The first pair of primers amplifies the endogenous locus targeted by the expression vector (SEQ ID NOs. 68 and 69). The second pair of primers (SEQ ID NOs. 59 and 60) amplifies a constant, or control region that is not targeted by the expression vector, so should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used was >30 to increase sensitivity. The most desired clones are those that yield a product for the constant region but not for the endogenous gene locus. Desired clones are also those that give weak-intensity endogenous locus products relative to the control reaction.

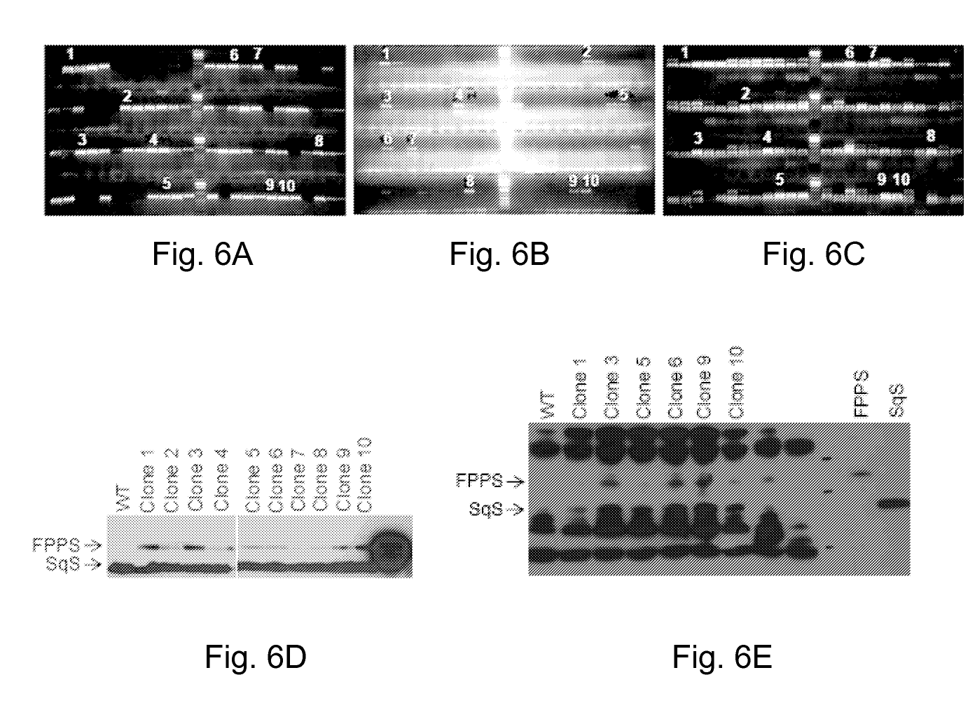

Results from this PCR on 96 clones were determined and the results are shown in FIG. 6. FIGS. 6A and 6B show PCR results using the pairs specific for the FPP synthase and squalene synthase genes, respectively. As can be seen, multiple transformed clones are positive for insertion of both the FPP synthase and squalene synthase genes (e.g. numbers 1-10). FIG. 6C shows the PCR results using the primer pairs to differentiate homoplasmic from heteroplasmic clones. As can be seen, multiple transformed clones are either homoplasmic or heteroplasmic to a degree in favor of incorporation of the transgene (e.g. numbers 1-10). Unnumbered clones demonstrate the presence of wild-type locus and, thus, were not selected for further analysis.

To ensure that the presence of the FPP synthase and squalene synthase genes led to expression of the FPP synthase and squalene synthase enzymes, a Western blot was performed. Approximately 1×10⁸ algae cells were collected from TAP agar medium and suspended in 0.5 ml of lysis buffer (750 mM Tris, pH=8.0, 15% sucrose, 100 mM beta-mercaptoethanol). Cells were lysed by sonication (5×30 sec at 15% power). Lysate was mixed 1:1 with loading buffer (5% SDS, 5% beta-mercaptoethanol, 30% sucrose, bromophenol blue) and proteins were separated by SDS-PAGE, followed by transfer to PVDF membrane. The membrane was blocked with TBST+5% dried, nonfat milk at 23° C. for 30 min, incubated with anti-FLAG antibody (diluted 1:1,000 in TBST+5% dried, nonfat milk) at 4° C. for 10 hours, washed three times with TBST, incubated with horseradish-linked anti-mouse antibody (diluted 1:10,000 in TBST+5% dried, nonfat milk) at 23° C. for 1 hour, and washed three times with TBST. Proteins were visualized with chemiluminescent detection. Results from multiple clones (FIG. 6D) show that expression of the FPP synthase gene in C. reinhardtii cells resulted in production of the protein. Visualization of the product of the squalene synthase gene was occluded by the signal from an unidentified protein present in all samples.

Cultivation of C. reinhardtii transformants for expression of endo-β-glucanase was carried out in liquid TAP medium at 23° C. under constant illumination of 5,000 Lux on a rotary shaker set at 100 rpm, unless stated otherwise. Cultures were maintained at a density of 1×10⁷ cells per ml for at least 48 hr prior to harvest.

To determine if the FPP synthase and squalene synthase enzymes were produced in transformed algae cells, both enzymes were immunopreciptated and visualized by Western blot. Briefly, 500 ml of algae cell culture was harvested by centrifugation at 4000×g at 4° C. for 15 min. The supernatant was decanted and the cells resuspended in 10 ml of lysis buffer (100 mM Tris-HCl, pH=8.0, 300 mM NaCl, 2% Tween-20). Cells were lysed by sonication (10×30 sec at 35% power). Lysate was clarified by centrifugation at 14,000×g at 4° C. for 1 hour. The supernatant was removed and incubated with anti-FLAG antibody-conjugated agarose resin at 4° C. for 10 hours. Resin was separated from the lysate by gravity filtration and washed 3× with wash buffer ((100 mM Tris-HCl, pH=8.0, 300 mM NaCl, 2% Tween-20). Results from Western blot analysis of multiple samples (FIGS. 6D and 6E) show that the both enzymes are indeed produced.

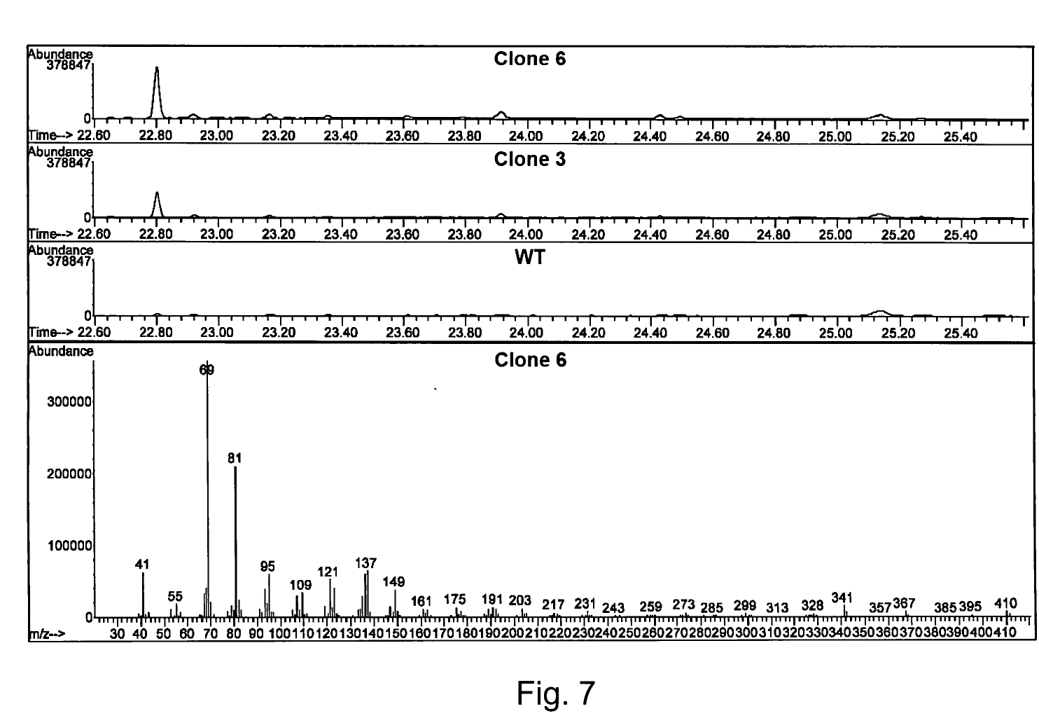

To determine whether FPP synthase and squalene synthase comprise a functional squalene biosynthesis pathway in vivo, the accumulation of squalene was determined. Briefly, 1.2 L of algae cell culture was harvested by centrifugation at 4000×g at 4° C. for 5 min. The supernatant was poured off and the remaining cell pellet resuspended in 10 ml of TAP medium and transferred to 50 mL centrifuge tube. Cells were pelleted again by centrifugation at 3,000 RPM for 5 minutes. All of the supernatant was removed and the cell mass determined. Samples were kept on ice and cell pellets resuspended in ice-cold methanol (MeOH) at a ratio of 0.75 mg biomass:5 mL MeOH. A solvent blank consisting of 30 mL MeOH was stored on ice in a 50 mL conical vial to control for leaching. Cell pellets were solubilized by repeated pipetting and lysates stored on ice to precipitate protein. Lysates were then clarified by centrifugation at 1,000 RPM for 5 min. 4 mL of soluble fraction was transferred to amber glass vials and overlaid with 8 mL of heptane. Lysates were extracted overnight at 23° C. on a rotating wheel. 1.5 mL of heptane from each sample was lyophilized to complete dryness, and then resuspended in 100 uL heptane. Analysis was performed on GC-MS. Results are shown in FIG. 7. These analyses were conducted with 5 replicates per strain.

### Example 3

### Production of Monoterpene Synthases in C. reinhardtii

In this example a nucleic acids encoding limonene synthase from M. spicata was introduced into C. reinhardtii. Transforming DNA is shown graphically in FIG. 3. In this instance the segment labeled “Transgene” is the gene encoding limonene synthase (SEQ ID NO. 74, Table 5; SEQ ID NO. 127, Table 6) that is regulated by the 5′ UTR and promoter sequence for the psbA gene from C. reinhardtii and the 3′ UTR for the psbA gene from C. reinhardtii, and the segment labeled “Selection Marker” is the kanamycin resistance encoding gene from bacteria, which is regulated by the 5′ UTR and promoter sequence for the atpA gene from C. reinhardtii and the 3′ UTR sequence for the rbcL gene from C. reinhardtii. The transgene cassette is targeted to the psbA loci of C. reinhardtii via the segments labeled “Homology A” and “Homology B,” which are identical to sequences of DNA flanking the psbA locus on the 5′ and 3′ sides, respectively. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain 137c (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells resuspended in 4 ml TAP medium for subsequent chloroplast transformation by particle bombardment (Cohen et al., supra, 1998). All transformations were carried out under kanamycin selection (100 μg/ml) in which resistance was conferred by the gene encoded by the segment in FIG. 3 labeled “Selection Marker.” (Chlamydomonas Stock Center, Duke University).

PCR was used to identify transformed strains. For PCR analysis, 106 algae cells (from agar plate or liquid culture) were suspended in 10 mM EDTA and heated to 95° C. for 10 minutes, then cooled to near 23° C. A PCR cocktail consisting of reaction buffer, MgCl2, dNTPs, PCR primer pair(s) (Table 4), DNA polymerase, and water was prepared. Algae lysate in EDTA was added to provide template for reaction. Magnesium concentration is varied to compensate for amount and concentration of algae lysate in EDTA added. Annealing temperature gradients were employed to determine optimal annealing temperature for specific primer pairs.

To identify strains that contain the limonene synthase gene, a primer pair was used in which one primer anneals to a site within the psbA 5′UTR (SEQ ID NO. 55) and the other primer anneals within the limonene synthase coding segment (SEQ ID NO. 56). Desired clones are those that yield a PCR product of expected size. To determine the degree to which the endogenous gene locus is displaced (heteroplasmic vs. homoplasmic), a PCR reaction consisting of two sets of primer pairs were employed (in the same reaction). The first pair of primers amplifies the endogenous locus targeted by the expression vector and consists of a primer that anneals within the psbA 5′UTR (SEQ ID NO. 57) and one that anneals within the psbA coding region (SEQ ID NO. 58). The second pair of primers (SEQ ID NOs. 59 and 60) amplifies a constant, or control region that is not targeted by the expression vector, so should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used was >30 to increase sensitivity. The most desired clones are those that yield a product for the constant region but not for the endogenous gene locus. Desired clones are also those that give weak-intensity endogenous locus products relative to the control reaction.

Cultivation of C. reinhardtii transformants for expression of limonene synthase was carried out in liquid TAP medium at 23° C. in the dark on a rotary shaker set at 100 rpm, unless stated otherwise. Cultures were maintained at a density of 1×10⁷ cells per ml for at least 48 hr prior to harvest.

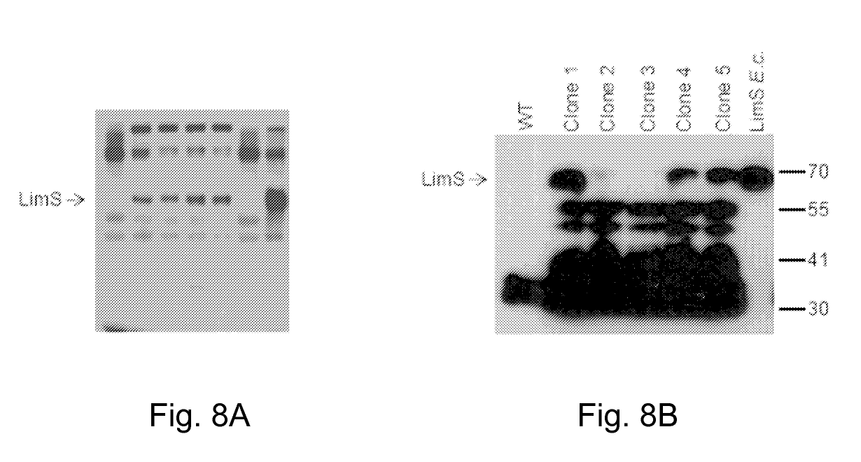

To determine if the limonene synthase gene led to expression of the limonene synthase in transformed algae cells, both soluble proteins were immunoprecipitated and visualized by Western blot. Briefly, 500 ml of algae cell culture was harvested by centrifugation at 4000×g at 4° C. for 15 min. The supernatant was decanted and the cells resuspended in 10 ml of lysis buffer (100 mM Tris-HCl, pH=8.0, 300 mM NaCl, 2% Tween-20). Cells were lysed by sonication (10×30 sec at 35% power). Lysate was clarified by centrifugation at 14,000×g at 4° C. for 1 hour. The supernatant was removed and incubated with anti-FLAG antibody-conjugated agarose resin at 4° C. for 10 hours. Resin was separated from the lysate by gravity filtration and washed 3× with wash buffer ((100 mM Tris-HCl, pH=8.0, 300 mM NaCl, 2% Tween-20). Results from Western blot analysis of multiple samples (FIG. 8) show that limonene synthase is indeed produced.

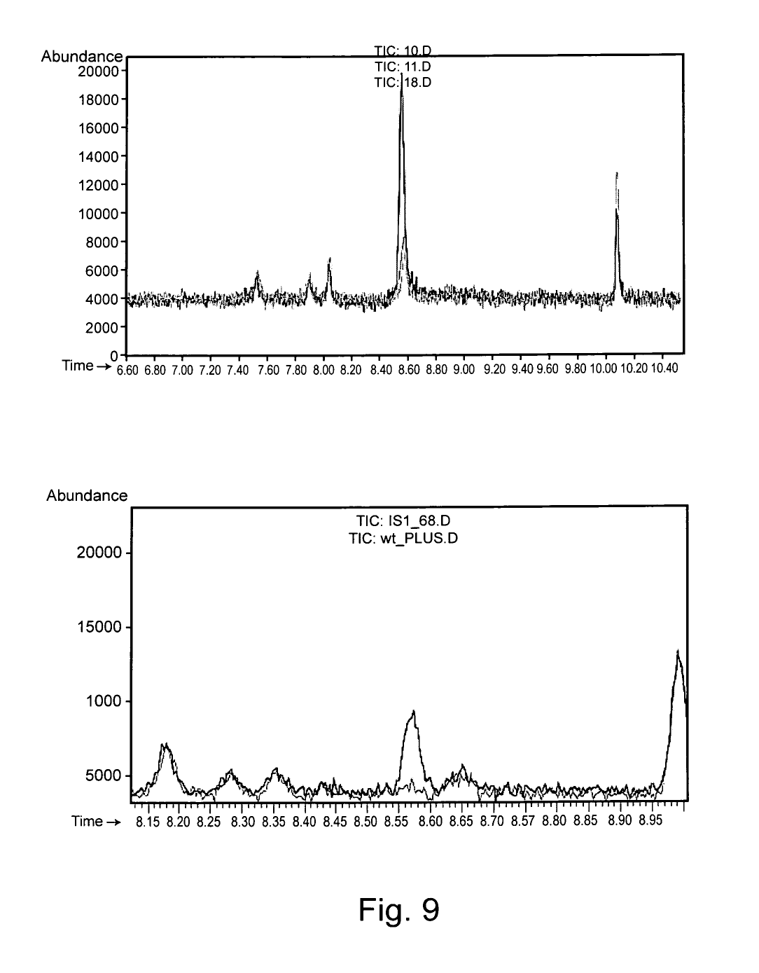

To determine whether limonene synthase produced in the algae chloroplast is a functional enzyme, limonene production from GPP was examined. Briefly, 50 uL of the limonene synthase-bound agarose (same samples prepared above) was suspended in 300 uL of reaction buffer (25 mM HEPES, pH=7.2, 100 mM KCl, 10 mM MnCl2, 10% glycerol, and 5 mM DTT) with 0.33 mg/mL GPP and transferred to a glass vial. The reaction was overlaid with heptane and incubated at 23° C. for 12 hours. The reaction was quenched and extracted by vortexing the mixture. 0.1 mL of heptane was removed and the sample was analyzed by GC-MS. Results are shown in FIG. 9. The results show that the isolated enzyme was capable of converting GPP to limonene in vitro.

Limonene synthase activity from crude cell lysates was also examined. Briefly, 50 mL of algae cell culture was harvested by centrifugation at 4000×g at 4° C. for 15 min. The supernatant was decanted and the cells resuspended in 0.5 mL of reaction buffer (25 mM HEPES, pH=7.2, 100 mM KCl, 10 mM MnCl2, 10% glycerol, and 5 mM DTT). Cells were lysed by sonication (10×30 sec at 35% power). 0.33 mg/mL of GPP was added to the lysate and the mixture was transferred to a glass vial. The reaction was overlaid with heptane and incubated at 23° C. for 12 hours. The reaction was quenched and extracted by vortexing the mixture. 0.1 mL of heptane was removed and the sample was analyzed by GC-MS. Results are shown in FIG. 9. The results show that the strain producing limonene synthase is capable of producing limonene in vivo.

### Example 4

### Production of GPP Synthases in C. reinhardtii

In this example a nucleic acids encoding GPP synthase from A. thaliana was introduced into C. reinhardtii. Transforming DNA is shown graphically in FIG. 3. In this instance the segment labeled “Transgene” is the gene encoding GPP synthase (SEQ ID NO. 89, Table 5; SEQ ID NO. 142, Table 6) that is regulated by the 5′ UTR and promoter sequence for the psbA gene from C. reinhardtii and the 3′ UTR for the psbA gene from C. reinhardtii, and the segment labeled “Selection Marker” is the kanamycin resistance encoding gene from bacteria, which is regulated by the 5′ UTR and promoter sequence for the atpA gene from C. reinhardtii and the 3′ UTR sequence for the rbcL gene from C. reinhardtii. The transgene cassette is targeted to the psbA loci of C. reinhardtii via the segments labeled “Homology A” and “Homology B,” which are identical to sequences of DNA flanking the psbA locus on the 5′ and 3′ sides, respectively. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain 137c (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells resuspended in 4 ml TAP medium for subsequent chloroplast transformation by particle bombardment (Cohen et al., supra, 1998). All transformations were carried out under kanamycin selection (100 μg/ml) in which resistance was conferred by the gene encoded by the segment in FIG. 3 labeled “Selection Marker.” (Chlamydomonas Stock Center, Duke University).

PCR was used to identify transformed strains. For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgCl2, dNTPs, PCR primer pair(s) (Table 4), DNA polymerase, and water was prepared. Algae lysate in EDTA was added to provide template for reaction. Magnesium concentration is varied to compensate for amount and concentration of algae lysate in EDTA added. Annealing temperature gradients were employed to determine optimal annealing temperature for specific primer pairs.

To identify strains that contain the GPP synthase gene, a primer pair was used in which one primer anneals to a site within the psbA 5′UTR (SEQ ID NO. 55) and the other primer anneals within the GPP synthase coding segment (SEQ ID NO. 61). Desired clones are those that yield a PCR product of expected size. To determine the degree to which the endogenous gene locus is displaced (heteroplasmic vs. homoplasmic), a PCR reaction consisting of two sets of primer pairs were employed (in the same reaction). The first pair of primers amplifies the endogenous locus targeted by the expression vector and consists of a primer that anneals within the psbA 5′UTR (SEQ ID NO. 57) and one that anneals within the psbA coding region (SEQ ID NO.58). The second pair of primers (SEQ ID NOs. 59 and 60) amplifies a constant, or control region that is not targeted by the expression vector, so should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used was >30 to increase sensitivity. The most desired clones are those that yield a product for the constant region but not for the endogenous gene locus. Desired clones are also those that give weak-intensity endogenous locus products relative to the control reaction. Results from this PCR are shown in FIGS. 10 (A and B).

To ensure that the presence of the GPP synthase gene led to expression of the GPP synthase, a Western blot was performed. Approximately 1×10⁸ algae cells were collected from TAP agar medium and suspended in 0.05 ml of lysis buffer (Bugbuster; Novagen). Solutions were heated to 95° C. for 5 min and then cooled to 23° C. Lysate was mixed 3:1 with loading buffer (XT Sample buffer; Bio-Rad), samples were heated to 95° C. for 1 min, cooled to 23° C., and insoluble proteins were removed by centrifugation. Soluble proteins were separated by SDS-PAGE, followed by transfer to PVDF membrane. The membrane was blocked with TBST+5% dried, nonfat milk at 23° C. for 30 min, incubated with anti-FLAG antibody (diluted 1:2,500 in TBST+5% dried, nonfat milk) at 4° C. for 10 hours, washed three times with TBST, incubated with horseradish-linked anti-mouse antibody (diluted 1:5,000 in TBST+5% dried, nonfat milk) at 23° C. for 1 hour, and washed three times with TBST. Proteins were visualized with chemiluminescent detection. Results from multiple clones (FIG. 10C) show that expression of the GPP synthase gene in C. reinhardtii cells resulted in production of the protein. FIG. 10C.

Cultivation of C. reinhardtii transformants for expression of GPP synthase was carried out in liquid TAP medium at 23° C. under constant illumination of 5,000 Lux on a rotary shaker set at 100 rpm, unless stated otherwise. Cultures were maintained at a density of 1×10⁷ cells per ml for at least 48 hr prior to harvest.

To determine whether GPP synthase produced in the algae chloroplast is a functional enzyme, limonene production from IPP and DMAPP is examined. Briefly, 50 mL of algae cell culture is harvested by centrifugation at 4000×g at 4° C. for 15 min. The supernatant is decanted and the cells resuspended in reaction buffer (25 mM HEPES, pH=7.2, 100 mM KCl, 10 mM MnCl2, 10% glycerol, and 5 mM DTT). Cells are lysed by sonication (10×30 sec at 35% power). One ug of limonene synthase (prepared from E. coli) is added to the lysate along with 0.33 mg/mL IPP and 0.33 mg/mL DMAPP and the mixture is transferred to a glass vial. The reaction is overlaid with heptane and incubated at 23° C. for 12 hours. The reaction is quenched and extracted by vortexing the mixture. 0.1 mL of heptane is removed and the sample analyzed by GC-MS.

### Example 5

### Production of FPP Synthases and Sesquiterpene Synthases in C. reinhardtii

In this example a nucleic acids encoding FPP synthase from G. gallus and zingiberene synthase from O. basilicum were introduced into C. reinhardtii. Transforming DNA is shown graphically in FIG. 3. In this instance the segment labeled “Transgene 1” is the gene encoding FPP synthase (SEQ ID NO. 82, Table 5; SEQ ID NO. 135, Table 6) that is regulated by the 5′ UTR and promoter sequence for the psbD gene from C. reinhardtii and the 3′ UTR for the psbA gene from C. reinhardtii, the segment labeled “Transgene 2” is the gene encoding zingiberene synthase (SEQ ID NO. 101, Table 5; SEQ ID NO. 154, Table 6) that is regulated by the 5′ UTR and promoter sequence for the psbD gene from C. reinhardtii and the 3′ UTR for the psbA gene from C. reinhardtii, and the segment labeled “Selection Marker” is the kanamycin resistance encoding gene from bacteria, which is regulated by the 5′ UTR and promoter sequence for the atpA gene from C. reinhardtii and the 3′ UTR sequence for the rbcL gene from C. reinhardtii. The transgene cassette is targeted to the 3HB locus of C. reinhardtii via the segments labeled “Homology C” and “Homology D,” which are identical to sequences of DNA flanking the 3HB locus on the 5′ and 3′ sides, respectively. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain 137c (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells resuspended in 4 ml TAP medium for subsequent chloroplast transformation by particle bombardment (Cohen et al., supra, 1998). All transformations were carried out under kanamycin selection (100 μg/ml) in which resistance was conferred by the gene encoded by the segment in FIG. 3 labeled “Selection Marker.” (Chlamydomonas Stock Center, Duke University).

PCR was used to identify transformed strains. For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgCl2, dNTPs, PCR primer pair(s) (Table 4), DNA polymerase, and water was prepared. Algae lysate in EDTA was added to provide template for reaction. Magnesium concentration is varied to compensate for amount and concentration of algae lysate in EDTA added. Annealing temperature gradients were employed to determine optimal annealing temperature for specific primer pairs. To identify strains that contain the FPP synthase gene, a primer pair was used in which one primer anneals to a site within the psbD 5′UTR (SEQ ID NO. 62) and the other primer anneals within the FPP synthase coding segment (SEQ ID NO. 66). To identify strains that contain the zingiberene synthase gene, a primer pair was used in which one primer anneals to a site within the psbD 5′UTR (SEQ ID NO. 62) and the other primer anneals within the zingiberene synthase coding segment (SEQ ID NO. 67). Desired clones are those that yield a PCR product of expected size in both reactions. To determine the degree to which the endogenous gene locus is displaced (heteroplasmic vs. homoplasmic), a PCR reaction consisting of two sets of primer pairs were employed (in the same reaction). The first pair of primers amplifies the endogenous locus targeted by the expression vector (SEQ ID NOs. 68 and 69). The second pair of primers (SEQ ID NOs. 59 and 60) amplifies a constant, or control region that is not targeted by the expression vector, so should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used was >30 to increase sensitivity. The most desired clones are those that yield a product for the constant region but not for the endogenous gene locus. Desired clones are also those that give weak-intensity endogenous locus products relative to the control reaction.

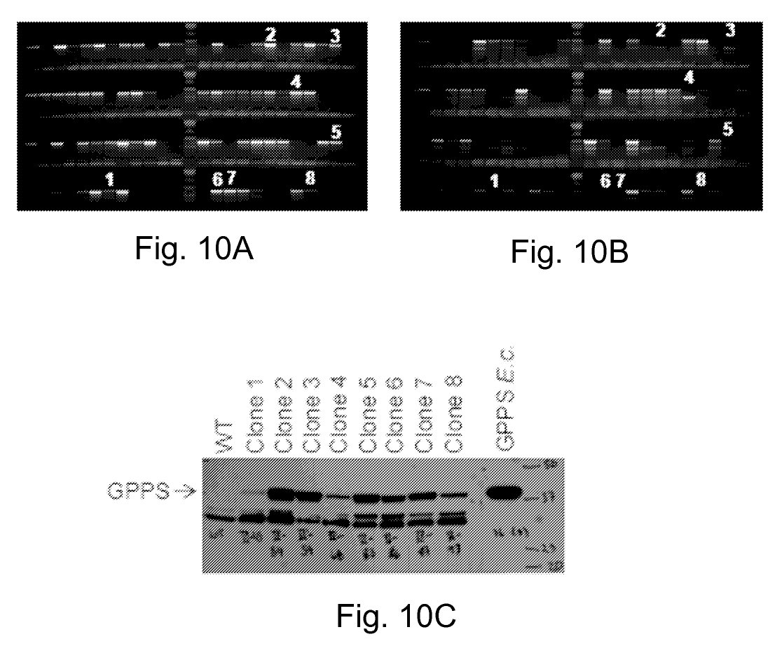

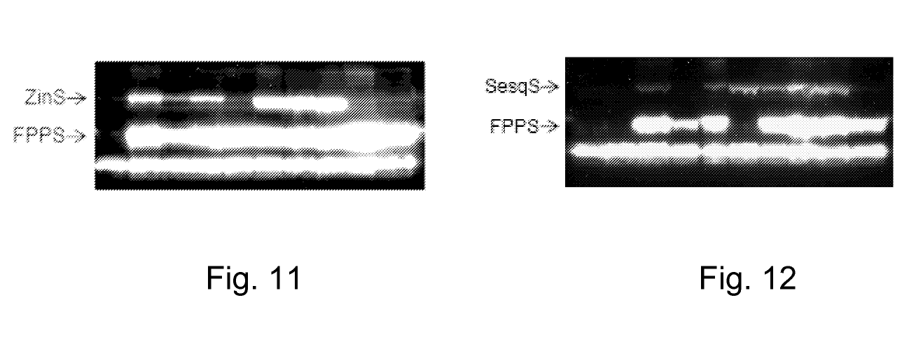

To ensure that the presence of the FPP synthase and zingiberene synthase genes led to expression of the FPP synthase and zingiberene synthase enzymes, a Western blot was performed. Approximately 1×10⁸ algae cells were collected from TAP agar medium and suspended in 0.05 ml of lysis buffer (Bugbuster; Novagen). Solutions were heated to 95° C. for 5 min and then cooled to 23° C. Lysate was mixed 3:1 with loading buffer (XT Sample buffer; Bio-Rad), samples were heated to 95° C. for 1 min, cooled to 23° C., and insoluble proteins were removed by centrifugation. Soluble proteins were separated by SDS-PAGE, followed by transfer to PVDF membrane. The membrane was blocked with TBST+5% dried, nonfat milk at 23° C. for 30 min, incubated with anti-FLAG antibody (diluted 1:2,500 in TBST+5% dried, nonfat milk) at 4° C. for 10 hours, washed three times with TBST, incubated with horseradish-linked anti-mouse antibody (diluted 1:5,000 in TBST+5% dried, nonfat milk) at 23° C. for 1 hour, and washed three times with TBST. Proteins were visualized with chemiluminescent detection. Results from multiple clones (FIG. 11) show expression of the GPP synthase gene in C. reinhardtii cells resulted in production of the protein.

Cultivation of C. reinhardtii transformants for expression of FPP synthase and zingiberene synthase was carried out in liquid TAP medium at 23° C. under constant illumination of 5,000 Lux on a rotary shaker set at 100 rpm, unless stated otherwise. Cultures were maintained at a density of 1×10⁷ cells per ml for at least 48 hr prior to harvest.

To determine whether FPP synthase and zingiberene synthase produced in the algae chloroplast are functional, sesquiterpene production from DMAPP and IPP is examined. Briefly, 50 mL of algae cell culture is harvested by centrifugation at 4000×g at 4° C. for 15 min. The supernatant is decanted and the cells resuspended in 0.5 mL of reaction buffer (25 mM HEPES, pH=7.2, 100 mM KCl, 10 mM MnCl2, 10% glycerol, and 5 mM DTT). Cells are lysed by sonication (10×30 sec at 35% power). 0.33 mg/mL of FPP are added to the lysate and the mixture transferred to a glass vial. The reaction is overlaid with heptane and incubated at 23° C. for 12 hours. The reaction is quenched and extracted by vortexing the mixture. 0.1 mL of heptane is removed and the sample analyzed by gas chromatography—mass spectrometry (GC-MS).

### Example 6

### Production of FPP Synthases and Sesquiterpene Synthases in C. reinhardtii

In this example a nucleic acids encoding FPP synthase from G. gallus and sesquiterpene synthase from Z. mays were introduced into C. reinhardtii. Transforming DNA is shown graphically in FIG. 3. In this instance the segment labeled “Transgene 1” is the gene encoding FPP synthase (SEQ ID NO. 82, Table 5; SEQ ID NO. 135, Table 6) that is regulated by the 5′ UTR and promoter sequence for the psbD gene from C. reinhardtii and the 3′ UTR for the psbA gene from C. reinhardtii, the segment labeled “Transgene 2” is the gene encoding sesquiterpene synthase (SEQ ID NO. 106, Table 5; SEQ ID NO. 159, Table 6) that is regulated by the 5′ UTR and promoter sequence for the psbD gene from C. reinhardtii and the 3′ UTR for the psbA gene from C. reinhardtii, and the segment labeled “Selection Marker” is the kanamycin resistance encoding gene from bacteria, which is regulated by the 5′ UTR and promoter sequence for the atpA gene from C. reinhardtii and the 3′ UTR sequence for the rbcL gene from C. reinhardtii. The transgene cassette is targeted to the 3HB locus of C. reinhardtii via the segments labeled “Homology C” and “Homology D,” which are identical to sequences of DNA flanking the 3HB locus on the 5′ and 3′ sides, respectively. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on C. reinhardtii strain 137c (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells resuspended in 4 ml TAP medium for subsequent chloroplast transformation by particle bombardment (Cohen et al., supra, 1998). All transformations were carried out under kanamycin selection (100 μg/ml) in which resistance was conferred by the gene encoded by the segment in FIG. 3 labeled “Selection Marker.” (Chlamydomonas Stock Center, Duke University).

PCR was used to identify transformed strains. For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgCl2, dNTPs, PCR primer pair(s) (Table 4), DNA polymerase, and water was prepared. Algae lysate in EDTA was added to provide template for reaction. Magnesium concentration is varied to compensate for amount and concentration of algae lysate in EDTA added. Annealing temperature gradients were employed to determine optimal annealing temperature for specific primer pairs.

To identify strains that contain the FPP synthase gene, a primer pair was used in which one primer anneals to a site within the psbD 5′UTR (SEQ ID NO. 62) and the other primer anneals within the FPP synthase coding segment (SEQ ID NO. 66). To identify strains that contain the sesquiterpene synthase gene, a primer pair was used in which one primer anneals to a site within the psbD 5′UTR (SEQ ID NO. 62) and the other primer anneals within the sesquiterpene synthase coding segment (SEQ ID NO. 70). Desired clones are those that yield a PCR product of expected size in both reactions. To determine the degree to which the endogenous gene locus is displaced (heteroplasmic vs. homoplasmic), a PCR reaction consisting of two sets of primer pairs were employed (in the same reaction). The first pair of primers amplifies the endogenous locus targeted by the expression vector (SEQ ID NOs. 68 and 69). The second pair of primers (SEQ ID NOs. 59 and 60) amplifies a constant, or control region that is not targeted by the expression vector, so should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used was >30 to increase sensitivity. The most desired clones are those that yield a product for the constant region but not for the endogenous gene locus. Desired clones are also those that give weak-intensity endogenous locus products relative to the control reaction.

To ensure that the presence of the FPP synthase and sesquiterpene synthase genes led to expression of the FPP synthase and sesquiterpene synthase enzymes, a Western blot was performed. Approximately 1×10⁸ algae cells were collected from TAP agar medium and suspended in 0.05 ml of lysis buffer (Bugbuster; Novagen). Solutions were heated to 95° C. for 5 min and then cooled to 23° C. Lysate was mixed 3:1 with loading buffer (XT Sample buffer; Bio-Rad), samples were heated to 95° C. for 1 min, cooled to 23° C., and insoluble proteins were removed by centrifugation. Soluble proteins were separated by SDS-PAGE, followed by transfer to PVDF membrane. The membrane was blocked with TBST+5% dried, nonfat milk at 23° C. for 30 min, incubated with anti-FLAG antibody (diluted 1:2,500 in TBST+5% dried, nonfat milk) at 4° C. for 10 hours, washed three times with TBST, incubated with horseradish-linked anti-mouse antibody (diluted 1:5,000 in TBST+5% dried, nonfat milk) at 23° C. for 1 hour, and washed three times with TBST. Proteins were visualized with chemiluminescent detection. Results from multiple clones (FIG. 12) show expression of the FPP synthase gene in C. reinhardtii cells resulted in production of the protein.

Cultivation of C. reinhardtii transformants for expression of FPP synthase and sesquiterpene synthase was carried out in liquid TAP medium at 23° C. under constant illumination of 5,000 Lux on a rotary shaker set at 100 rpm, unless stated otherwise. Cultures were maintained at a density of 1×10⁷ cells per ml for at least 48 hr prior to harvest.

To determine whether FPP synthase and sesquiterpene synthase produced in the algae chloroplast are functional, sesquiterpene production from DMAPP and IPP is examined. Briefly, 50 mL of algae cell culture is harvested by centrifugation at 4000×g at 4° C. for 15 min. The supernatant is decanted and the cells resuspended in 0.5 mL of reaction buffer (25 mM HEPES, pH=7.2, 100 mM KCl, 10 mM MnCl2, 10% glycerol, and 5 mM DTT). Cells are lysed by sonication (10×30 sec at 35% power). 0.33 mg/mL of FPP are added to the lysate and the mixture transferred to a glass vial. The reaction is overlaid with heptane and incubated at 23° C. for 12 hours. The reaction is quenched and extracted by vortexing the mixture. 0.1 mL of heptane is removed and the sample analyzed by gas chromatography—mass spectrometry (GC-MS).

### Example 7

### Production of Diterpene Molecules in C. reinhardtii

In this example, strains of C. reinhardtii were engineered to express FPP synthase from G. gallus and squalene synthase from S. aureus (as described above). Cultivation of C. reinhardtii transformants for expression of FPP synthase and squalene synthase was carried out in liquid HSM medium at 23° C. under constant illumination of 5,000 Lux on a rotary shaker set at 100 rpm, unless stated otherwise.

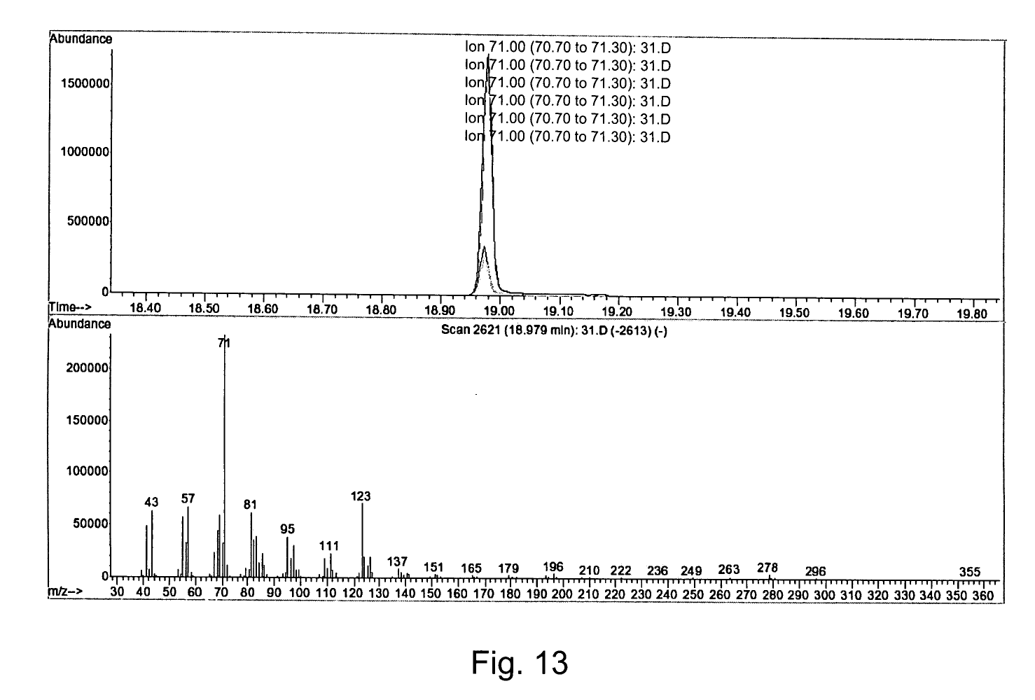

To determine if expression of either enzyme impacts the metabolic pathways that produce diterpenes, phytol production was examined. Briefly, 800 mL of algae cell culture was harvested by centrifugation at 4000×g at 4° C. for 5 min. The supernatant was poured off and the remaining cell pellet resuspended in 10 ml of HSM medium and transferred to 50 mL centrifuge tube. Cells were pelleted again by centrifugation at 3,000 RPM for 5 minutes. All of the supernatant was removed and the cell mass determined. Samples were maintained at 23° C. and cell pellets resuspended in MeOH:KOH (1:10) at a ratio of 0.75 mg biomass:5 mL MeOH. A solvent blank consisting of 30 mL MeOH:KOH (1:10) was stored in a 50 mL conical vial to control for leaching. Cell pellets were solubilized by repeated pipetting. Lysates were heated to 55° C. for 30 minutes (shaken at 10 minute intervals to ensure complete mixing). Lysates were cooled to approximately 23° C. and 4 mL of each samples was transferred to amber glass vials and overlaid with 8 mL of heptane and mixed for 10-12 hours at 23° C. on a rotating wheel. 100 uL of heptane was collected. Analysis was performed on GC-MS. Results are shown in FIG. 13. The results show that expression of these enzymes increases the production of phytol in C. reinhardtii.

### Example 8

### Production of Enzymes Comprising a Monoterpene Biosynthesis Pathway in Escherichia coli

In this example a nucleic acids encoding GPP synthase from A. thaliana and limonene synthase from M. spicata were introduced into E. coli BL-21 cells. In this instance the gene encoding GPP synthase (SEQ ID NO. 89, Table 5; SEQ ID NO. 142, Table 6) and the gene encoding limonene synthase (SEQ ID NO. 74, Table 5; SEQ ID NO. 127, Table 6) were each ligated into the plasmid pET-21a using the NdeI and XhoI sites. The resulting plasmid was transformed into E. coli BL-21 cells. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

Expression of the synthases was induced when cell density reached OD=0.6. Cells were grown at 30° C. for 5 hours and then harvested. To ensure that the presence of the GPP synthase and limonene synthase genes led to expression of the enzymes, a Western blot was performed essentially as described above. Results (FIG. 14; lane 1: MW ladder; lane 2: GPP synthase; and lane 3: limonene synthase) show expression of the GPP synthase and limonene synthase proteins in E. coli cells.

To determine whether the enzymes were functional, limonene production from IPP and DMAPP was examined. Briefly, 500 ml of E. coli cell culture was harvested by centrifugation at 4000×g at 4° C. for 15 min. The supernatant was decanted and the cells resuspended in 10 ml of lysis buffer (100 mM Tris-HCl, pH=8.0, 300 mM NaCl, 2% Tween-20). Cells were lysed by sonication (3×30 sec at 35% power). Lysate was clarified by centrifugation at 14,000×g at 4° C. for 1 hour. The supernatant was removed and incubated with anti-FLAG antibody-conjugated agarose resin at 4° C. for 10 hours. Resin was separated from the lysate by gravity filtration and washed 3× with wash buffer (100 mM Tris-HCl, pH=8.0, 300 mM NaCl, 2% Tween-20). Enzymes were eluted from the resin with elution buffer (100 mM Tris-HCl, pH=8.0, 300 mM NaCl, 250 μg/mL FLAG peptide).

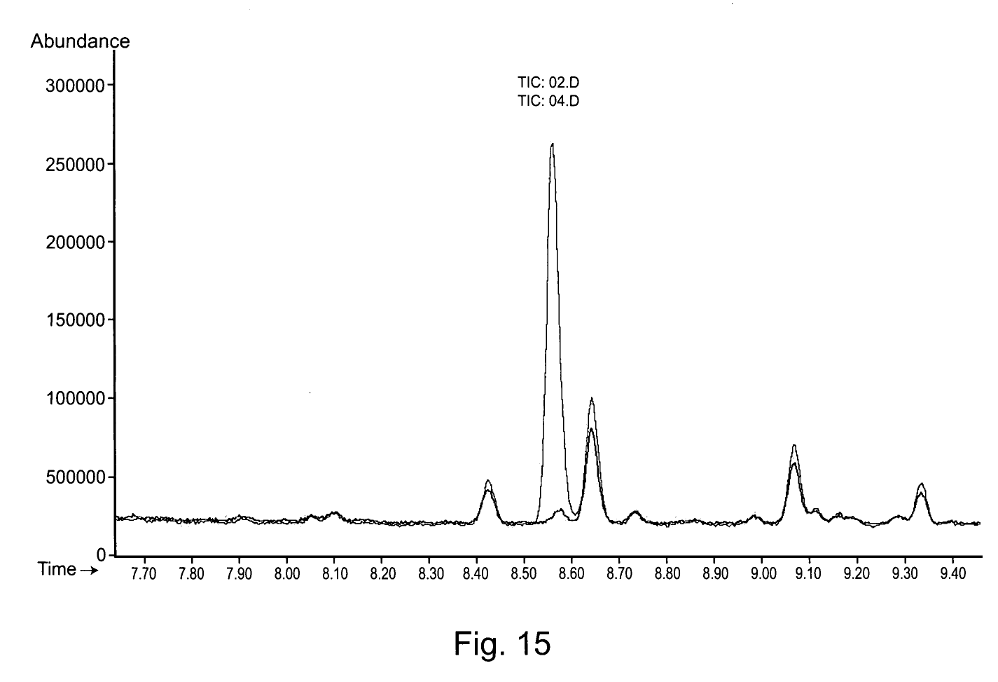

Reactions were carried out in reaction buffer (25 mM HEPES, pH=7.2, 100 mM KCl, mM MnCl2, 10% glycerol, and 5 mM DTT), with or without the addition of limonene synthase, and IPP and DMAPP. The reaction was overlaid with heptane and incubated at 23° C. for 12 hours. The reaction was quenched and extracted by vortexing the mixture. 0.1 mL of heptane is removed and the sample analyzed by gas chromatography—mass spectrometry (GC-MS). Results are shown in FIG. 15. A large peak resulted in the reaction containing the limonene synthase isolated from the transformed E. coli strain.

### Example 9

### Nuclear Transformation of C. reinhardtii with a Nucleic Acid Encoding a Fused Resistance Marker and Gene of Interest

In this example, a nucleic acid encoding xylanase 2 from T. reesei is introduced into C. reinhardtii. Transforming DNA is shown graphically in FIG. 16A. The segment labeled “Transgene” is xylanase 2 encoding gene, the segment labeled “Promoter/5′ UTR” is the C. reinhardtii HSP70/rbcS2 5′ UTR with introns, the segment labeled “Selectable Marker” is a bleomycin resistance gene, the segment labeled CM (cleavage moiety) is the A2 viral protease of foot and mouth disease virus (FMDV), and the segment labeled 3′ UTR is the 3′UTR from C. reinhardtii rbcS2. The bleomycin resistance gene, A2 and xylanase 2 coding regions are physically linked in-frame, resulting in a chimeric single ORF. A Metal Affinity Tag (MAT) and FLAG epitope tag were added to the 3′ end of the ORF, using standard techniques. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations are carried out on cell-wall-deficient C. reinhardtii strain CC3395 (an arginine auxotrophic mutant mt−). Cells were grown and transformed via electroporation. Cells are grown to mid-log phase (approximately 2-6×10⁶ cells/ml). Tween-20 was added into cell cultures to a concentration of 0.05% before harvest to prevent cells from sticking to centrifugation tubes. Spin cells down gently (between 2000 and 5000 g) for 5 min. The supernatant was removed and cells resuspended in TAP+40 mM sucrose media. 1 to 2 ug of transforming DNA was mixed with ˜1×10⁸ cells on ice and transferred to electroporation cuvettes. Electroporation was performed with the capacitance set at 25 uF, the voltage at 800 V to deliver V/cm of 2000 and a time constant for 10-14 ms. Following electroporation, the cuvette is returned to room temperature for 5-20 min. Cells were transferred to 10 ml of TAP+40 mM sucrose+50 ug/ml arginine and allowed to recover at room temperature for 12-16 hours with continuous shaking. Cells were then harvested by centrifugation at between 2000 g and 5000 g and resuspended in 0.5 ml TAP+40 mM sucrose medium. 0.25 ml of cells were plated on TAP+100 ug/ml bleomycin+50 ug/ml arginine. All transformations were carried out under bleomycin selection (100 μg/ml) in which resistance was conferred by the gene encoded by the segment in FIG. 16A labeled “Selection Marker.” Transformed strains are maintained in the presence of bleomycin to prevent loss of the exogenous DNA.

Colonies growing in the presence of bleomycin were screened by dot blot. Briefly, colonies were lysed by BugBuster Protein Extraction Reagent (Novagen) and MAT-tagged proteins were separated using Co2+ magnetic beads (Invitrogen), according to manufacturer's instructions. After exposure to the proteins, the beads were washed three times by 150 ul of 1× Tris Buffered Saline with 0.05% Tween-20 (TBST) at room temperature. Proteins were released from beads by 150 ul 10 uM EDTA, 25 mM Tris-HCl pH 7.0, 400 mM NaCl, and the 150 ul eluates were dot blotted onto nitrocellulose membranes. Membranes were blocked by Starting Block (TBS) blocking buffer (Thermo Scientific) and probed for one hour with mouse anti-FLAG antibody-horseradish peroxidase conjugate (Sigma) diluted 1:3000 in Starting Block buffer. After probing, membranes were washed four times with TBST, then developed with Supersignal West Dura chemiluminescent subrate (Thermo Scientific) and imaged using a CCD camera (Alpha Innotech). Colonies showing positive results in the dot blot analysis are then screened by western blotting.

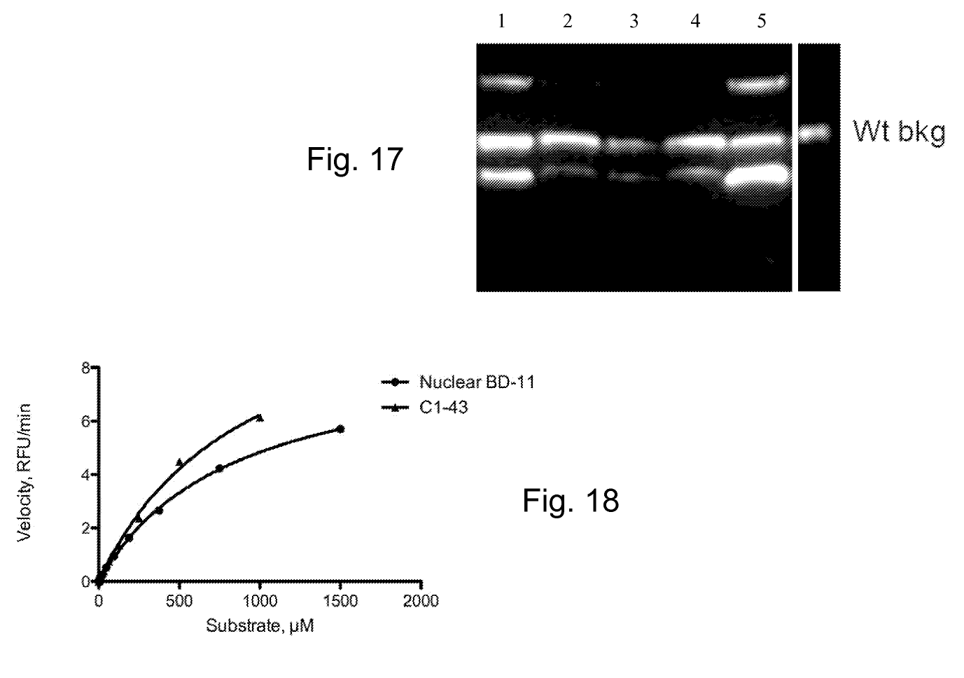

Patches of algae cells growing on TAP agar plates were lysed by resuspending cells in 50 ul of 1×SDS sample buffer with reducing agent (BioRad). Samples were then boiled and run on a 10% Bis-tris polyacrylamide gel (BioRad) and transferred to PVDF membranes using a Trans-blot semi-dry blotter (BioRad) according to manufacturers instructions. Membranes were blocked by Starting Block (TBS) blocking buffer (Thermo Scientific) and probed for one hour with mouse anti-FLAG antibody-horseradish peroxidase conjugate (Sigma) diluted 1:3000 in Starting Block buffer. After probing, membranes were washed four times with TBST, then developed with Supersignal West Dura chemiluminescent subrate (Thermo Scientific) and imaged using a CCD camera (Alpha Innotech). Results from 5 colonies (and wild-type control) are shown in FIG. 17. Positive colonies show a band with a lower molecular weight than the WT background. A small amount of intact fusion (Bleomycin resistance marker fused to xylanase) is translated by the cells; as the resistance marker forms a dimer, these products migrate at a higher molecular weight. The results indicate that xylanase 2 is being produced from the strains.

To determine whether xylanase produced is functional, enzyme activity is examined. Patches of cells were homogenized by 50 ul by BugBuster Protein Extraction Reagent (Novagen) and EnzCheck Ultra Xylanase Assay Kit (Molecular Probe) was used to examine xylanase activity according to manufacturer's instructions.

Results are shown in FIG. 18 and are compared with xylanase isolated from a C. reinhardtii strain producing exogenous xylanase from a transformed chloroplast.

Similar protocols for plasmid construction, transformation, colony selection Western blot, and enzyme analysis were performed with each of the enzymes listed in Table 3. For each of these enzymes, experiments were repeated where the fusion protein was prepared with the C. reinhardtii carbonic anhydrase secretion signal.

TABLE 3
Select enzymes expressed from the nucleus in C. reinhardtii

| Enzyme | Source |
|---|---|
| CBH1 | T. viride |
| CBHII | T. Reesei |
| CBH1 | A. aculeatus |
| Endoglucanase I | T. Reesei |
| Endoglucanase III | T. Reesei |
| Endoglucanase V | T. reesei |
| Endoglucanase A | A. niger |
| beta-D-glucoside | T. reesei |
| glucohydrolase | |
| beta-glucosidase | T. reesei |
| Beta glucosidase | A. niger |
| Xylanase 2 | T. reesei |
| Xylanase 1 | T. reesei |
| FPP synthase | Chicken |
| Squalene desaturase | S. aureus |

### Example 10

### Nuclear Transformation of C. reinhardtii with a Nucleic Acids Encoding a Resistance Marker and a Gene of Interest

In this example, a nucleic acid encoding endoglucanase from T. reesei is introduced into C. reinhardtii. Transforming DNA is shown graphically in FIG. 16B. The segment labeled “Transgene” is the endoglucanase encoding gene, the segment labeled “Promoter/5′ UTR” is the C. reinhardtii HSP70/rbcS2 5′ UTR, the segment labeled “Selectable Marker” is a hygromycin resistance gene (should we include statement regarding non-functional paromomycin resistance gene?maybe not I think), and the segment labeled 3′ UTR is the 3′UTR from C. reinhardtii rbcS2. The hygromycin resistance gene and endoglucanase coding regions are expressed separately with hygromycin marker driven by a beta 2-tubulin promoter Endoglucanase coding sequence was fused to a DNA fragment that encodes a C. reinhardtii carbonic anhydrase secretion signal. A Metal Affinity Tag (MAT) and FLAG epitope tag were added to the 3′ end of the ORF, using standard techniques. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations are carried out on cell-wall strain C. reinhardtii strain CC1960 (mt+), essentially as described above. All transformations were carried out under hygromycin selection (20 μg/ml) in which resistance was conferred by the gene encoded by the segment in FIG. 16B labeled “Selection Marker.” Transformed strains are maintained in the presence of hygromycin.

Colonies growing in the presence of hygromycin were screened by dot blot. Briefly, colonies were lysed and MAT-tagged proteins were purified for dot blots as previously described. After exposure to the proteins, the beads were washed two times with 150 ul of 1×TBST and one time with 150 ul of 100 mM Tris-HCl pH 7.5, 400 mM NaCl, and 20 mM Imidazole at room temperature. Proteins were released from beads by 150 ul 10 uM EDTA, 25 mM Tris-HCl pH 7.0, 400 mM NaCl and the 150 ul eluates were dot blotted onto nitrocellulose membranes. Membranes were blocked and probed with anti-FLAG antibody as previously described.

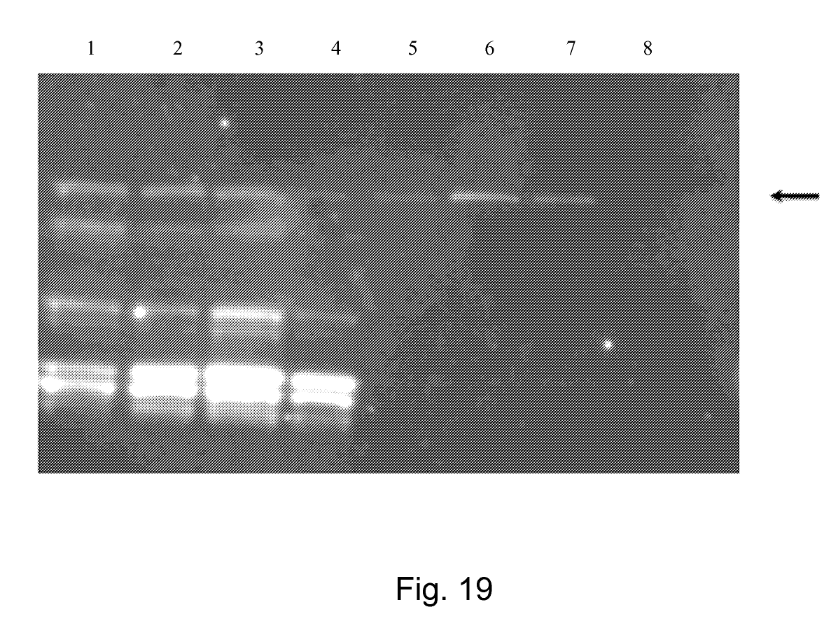

Colonies showing positive results in the dot blot analysis are then screened by western blotting. 1×10⁸ cells from log phase liquid cultures were harvested at 3000×g at for 5 min. The supernatant was decanted and the cells resuspended in 1 ml of protein binding buffer (100 mM Tris-HCl, pH=7.5, 400 mM NaCl, 20 mM Imidazole). Cells were lysed by vortexing in the presence of 500 ul zirconium beads at the highest speed (1 mm, BioSpec Products, Inc.). Lysate was clarified by centrifugation at 2400 g at 4° C. for 1 min. The supernatant was removed and incubated with Ni2+ agarose resin (Invitrogen) at 4° C. for 1 hour. Resin was separated from the lysate by centrifugation at 2400 g and washed 3× with protein binding buffer (100 mM Tris-HCl, pH=7.5, 400 mM NaCl, 20 mM Imidazole). Proteins were eluted by incubation of the resin with 100 ul elution buffer (25 mM Tris-HCl, pH=7.5, 400 mM NaCl, 20 mM EDTA). Results from 3 colonies (and wild-type control) from samples bound to the resin (lanes 1-4) and samples after elution (lanes 5-8) are shown in FIG. 19. Positive colonies show a band (indicated by arrow) that is missing in wild type control. The results indicate that endo-glucanase is being produced from the strains.

### Example 11

### Nuclear Transformation of C. reinhardtii with a Nucleic Acids Encoding a Resistance Marker and a Gene of Interest

In this example, a nucleic acid encoding CBH1 from A. aculeatus is introduced into C. reinhardtii. Transforming DNA is shown graphically in FIG. 16B. The segment labeled “Transgene” is the exoglucanase encoding gene, the segment labeled “Promoter/5′ UTR” is the C. reinhardtii HSP70/rbcS2 5′ UTR, the segment labeled “Selectable Marker” is a hygromycin resistance gene, and the segment labeled 3′ UTR is the 3′UTR from C. reinhardtii rbcS2. The hygromycin resistance gene and CBH1 coding regions were expressed separately with hygromycin marker driven by a beta-2 tubulin promoter. CBH1 coding sequence was fused to a DNA fragment that encodes a C. reinhardtii carbonic anhydrase secretion signal. A Metal Affinity Tag (MAT) and FLAG epitope tag were added to the 3′ end of the ORF, using standard techniques. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations are carried out on cell-wall strain C. reinhardtii strain CC1960 (mt+), essentially as described above. All transformations were carried out under hygromycin selection (20 μg/ml) in which resistance was conferred by the gene encoded by the segment in FIG. 16B labeled “Selection Marker.” Transformed strains are maintained in the presence of hygromycin.

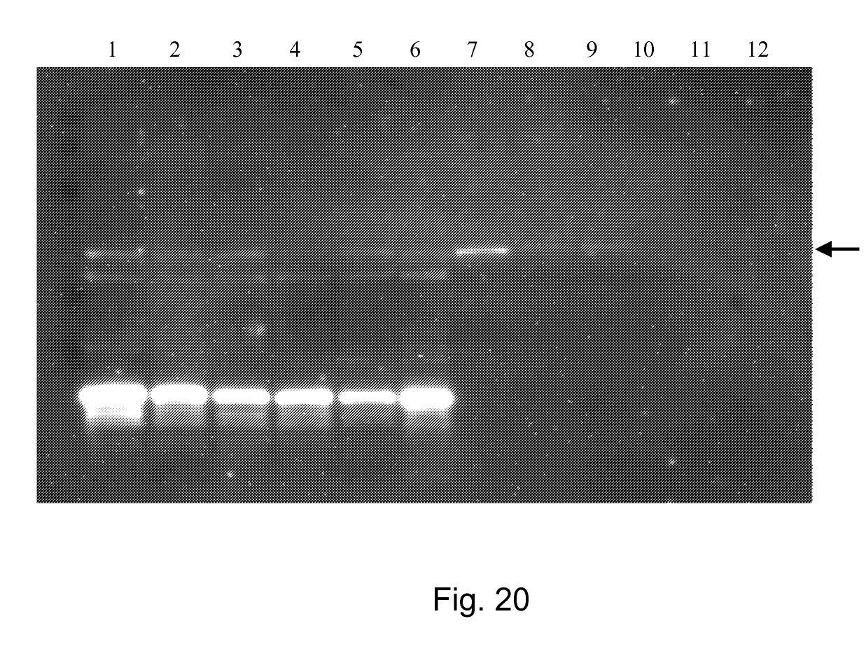

Colonies growing in the presence of hygromycin were screened by pull-down assay followed by Western blot analysis. Four ml of liquid cultures was harvested at 3000×g at for 5 min. The supernatant was decanted and the cells resuspended in 1 ml of protein binding buffer (100 mM Tris-HCl, pH=7.5, 400 mM NaCl, 20 mM Imidazole, 0.005% NP40). The protein purification and Western blot analysis were carried out as described above. Results from 5 colonies (and wild-type control) from samples bound to the resin (lanes 1-5) and samples after elution (lanes 7-11) are shown in FIG. 20. Positive colonies (7, 8, 9) show a band (indicated by arrow) that is missing in wild type control. The results indicate that CBH1 is being produced from the strains.

### Example 12

### Nuclear Transformation of C. reinhardtii with a Nucleic Acids Encoding a Resistance Marker and a Secreted Gene of Interest

In this example, a nucleic acid encoding Xylanase 2 from T. reesei is introduced into C. reinhardtii. Transforming DNA is shown graphically in FIG. 16A. The segment labeled “Transgene” is xylanase 2 encoding gene, the segment labeled “Promoter/5′ UTR” is the C. reinhardtii HSP70/rbcS2 5′ UTR with introns (intron position not indicated), the segment labeled “Selectable Marker” is a bleomycin resistance gene, the segment labeled CM (cleavage moiety) is the A2 viral protease of foot and mouth disease virus (FMDV), and the segment labeled 3′ UTR is the 3′UTR from C. reinhardtii rbcS2. The bleomycin resistance gene, A2 and xylanase 2 coding regions are physically linked in-frame, resulting in a chimeric single ORF. Nucleic acids encoding the secretion signal sequence of the Chlamydomonas carbonic anhydrase gene were placed between the A2 sequence and the 5′ end of the xylanase encoding sequence. A Metal Affinity Tag (MAT) and FLAG epitope tag were added to the 3′ end of the ORF, using standard techniques. All DNA manipulations carried out in the construction of this transforming DNA were essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, all transformations were carried out on cell-wall-deficient C. reinhardtii strain CC3395 (an arginine auxotrophic mutant mt−). Cells were grown and transformed via electroporation. Cells are grown to mid-log phase (approximately 2-6×10⁶ cells/ml). Tween-20 was added into cell cultures to a concentration of 0.05% before harvest to prevent cells from sticking to centrifugation tubes. Spin cells down gently (between 2000 and 5000 g) for 5 min. The supernatant was removed and cells resuspended in TAP+40 mM sucrose media. 1 to 2 ug of transforming DNA was mixed with ˜1×10⁸ cells on ice and transferred to a electroporation cuvettes. Electroporation was performed with the capacitance set at 25 uF, the voltage at 800 V to deliver V/cm of 2000 and a time constant for 10-14 ms. Following electroporation, the cuvette is returned to room temperature for 5-20 min. Cells were transferred to 10 ml of TAP+40 mM sucrose+50 ug/ml arginine and allowed to recover at room temperature for 12-16 hours with continuous shaking. Cells were then harvested by centrifugation at between 2000 g and 5000 g and resuspendeded in 0.5 ml TAP+40 mM sucrose medium. 0.25 ml of cells were plated on TAP+100 ug/ml bleomycin+50 ug/ml arginine. All transformations were carried out under bleomycin selection (100 μg/ml) in which resistance was conferred by the gene encoded by the segment in FIG. 16A labeled “Selection Marker.” Transformed strains are maintained in the presence of bleomycin to prevent loss of the exogenous DNA.

Colonies growing in the presence of bleomycin were screened by dot blot. Briefly, colonies were lysed by BugBuster Protein Extraction Reagent (Novagen) and MAT-tagged proteins were separated using Co2+ magnetic beads (Invitrogen), according to manufacturers instructions. After exposure to the proteins, the beads were washed three times by 150 ul of 1× Tris Buffered Saline with 0.05% Tween-20 (TBST) at room temperature. Proteins were released from beads by 150 ul 10 mM EDTA, 25 mM Tris-HCl pH 7.0, 400 mM NaCl, and the 150 ul eluates were dot blotted onto nitrocellulose membranes. Membranes were blocked by Starting Block (TBS) blocking buffer (Thermo Scientific) and probed for one hour with mouse anti-FLAG antibody-horseradish peroxidase conjugate (Sigma) diluted 1:3000 in Starting Block buffer. After probing, membranes were washed four times with TBST, then developed with Supersignal West Dura chemiluminescent subrate (Thermo Scientific) and imaged using a CCD camera (Alpha Innotech). Colonies showing positive results in the dot blot analysis are then screened by western blotting. Algae cells were cultured at a volume of 50 ml, and carefully centrifuged to avoid cell lysis. Cobalt-derivatized magnetic beads (Invitrogen) were added to the conditioned media, and incubated, and washed according to manufacturer instructions. Proteins were released from beads by 150 ul 10 mM EDTA, 25 mM Tris-HCl pH 7.0, 400 mM NaCl, and 50 ul of 4×SDS sample buffer with reducing agent (BioRad) was added to the eluates. Samples were then boiled and run on a 10% Bis-tris polyacrylamide gel (BioRad) and transferred to PVDF membranes using a Trans-blot semi-dry blotter (BioRad) according to manufacturers instructions. Membranes were blocked by Starting Block (TBS) blocking buffer (Thermo Scientific) and probed for one hour with mouse anti-FLAG antibody-horseradish peroxidase conjugate (Sigma) diluted 1:3000 in Starting Block buffer. After probing, membranes were washed four times with TBST, then developed with Supersignal West Dura chemiluminescent subrate (Thermo Scientific) and imaged using a CCD camera (Alpha Innotech).

TABLE 4
PCR Primers

| SEQ ID | | |
|---|---|---|
| NO. | Use | Sequence |
| 55 | psbA 5′ UTR forward primer | GTGCTAGGTAACTAACGTTTGATTTTT |
| 56 | Limonene synthase reverse | TGGGTTCATATCTGGACGTT |
| | primer (155) | |
| 57 | psbA 5′ UTR forward primer | GGAAGGGGACGTAGGTACATAAA |
| | (wild-type) | |
| 58 | psbA 3′ reverse primer | TTAGAACGTGTTTTGTTCCCAAT |
| | (wild-type) | |
| 59 | Control forward primer | CCGAACTGAGGTTGGGTTTA |
| 60 | Control reverse primer | GGGGGAGCGAATAGGATTAG |
| 61 | GPP synthase reverse primer | GCAACTTCAGCTGTTTGACCT |
| | (281) | |
| 62 | psbD 5′ UTR forward primer | AAATTTAACGTAACGATGAGTTG |
| 63 | psbA 3′ reverse primer | TTAGAACGTGTTTTGTTCCCAAT |
| | (wild-type) | |
| 64 | psbC 5′ UTR forward primer | TGGTACAAGAGGATTTTTGTTGTT |
| 65 | psbD 5′ UTR forward primer | AAATTTAACGTAACGATGAGTTG |
| 66 | FPP synthase reverse primer | CGTTCTTCTGAGAAATGGCTTA |
| | (163) | |
| 67 | Zingiberene synthase reverse | ATTAGCATCAGAGCCGCATT |
| | primer (293) | |
| 68 | 3HB forward primer (wild- | CTCGCCTATCGGCTAACAAG |
| | type) | |
| 69 | 3HB forward primer (wild- | CACAAGAAGCAACCCCTTGA |
| | type) | |
| 70 | Sesquiterpene synthase | CGACGGAATAAAGGTGTACGA |
| | reverse primer (298) | |
| 71 | psbC 5′ UTR forward primer | TGGTACAAGAGGATTTTTGTTGTT |
| 72 | Squalene synthase reverse | TCAGCAATTGCAGCGTAATA |
| | primer (166) | |
| 73 | Bisabolene synthase reverse | GACGTTCTTGACGTTTTGTTTG |
| | primer (307) | |

TABLE 5
Nucleic acids encoding exemplary isoprenoid producing enzymes.

| | | |
|---|---|---|
| | | Enzyme |
| SEQ | | (synthase) |
| ID NO | Codon-biased, Synthesized Gene Sequence | encoded |

| | | |
|---|---|---|
| 74 | ATGGTACCAAGACGTTCAGGTAACTATAATCCTAGCCGTTGGGACGT | Limonene |
| | AAATTTCATTCAATCTTTATTATCTGATTATAAAGAAGATAAACACG | (M. spicata) |
| | TTATTAGAGCTTCTGAATTAGTAACACTTGTTAAGATGGAATTAGAA | |
| | AAAGAAACAGATCAAATCCGTCAATTAGAATTAATTGACGATTTAC | |
| | AACGTATGGGTTTATCTGATCATTTCCAAAACGAATTTAAAGAAATC | |
| | TTATCAAGTATTTACTTAGATCATCATTATTACAAAAATCCATTTCCA | |
| | AAAGAAGAGCGTGATTTATACTCAACTAGCTTAGCTTTTCGTTTATT | |
| | ACGTGAACACGGTTTTCAAGTAGCACAAGAAGTTTTTGATTCATTCA | |
| | AAAATGAAGAGGGTGAATTTAAGGAGAGCTTATCTGACGATACTCG | |
| | TGGCTTATTACAATTATATGAAGCATCATTCTTATTAACAGAGGGTG | |
| | AAACAACCTTAGAAAGTGCACGCGAATTTGCTACAAAATTTTTAGA | |
| | AGAAAAAGTTAACGAAGGTGGCGTTGATGGTGACTTATTAACAAGA | |
| | ATTGCTTACTCATTAGATATTCCCTTACATTGGCGCATTAAACGTCCT | |
| | AATGCCCCAGTTTGGATTGAATGGTATCGTAAACGTCCAGATATGAA | |
| | CCCAGTGGTTTTAGAATTAGCAATTTTAGACTTAAACATTGTACAAG | |
| | CTCAATTTCAAGAGGAATTAAAAGAGTCTTTTCGCTGGTGGCGTAAT | |
| | ACTGGTTTTGTTGAGAAATTACCATTTGCACGTGATCGTTTAGTTGA | |
| | ATGTTACTTTTGGAACACTGGTATTATTGAACCACGTCAACACGCAT | |
| | CAGCTCGTATTATGATGGGTAAAGTAAATGCATTAATTACAGTAATT | |
| | GATGACATCTATGATGTTTATGGAACACTTGAAGAATTAGAACAATT | |
| | CACTGATTTAATTCGCAGATGGGACATAAACTCAATAGATCAATTAC | |
| | CAGATTATATGCAATTATGTTTTCTTGCATTAAACAATTTCGTTGATG | |
| | ACACTTCATACGATGTTATGAAAGAAAAGGGTGTTAATGTTATTCCT | |
| | TACTTACGTCAATCTTGGGTAGACCTTGCAGACAAATATATGGTAGA | |
| | AGCACGTTGGTTCTACGGTGGCCATAAACCATCATTAGAAGAATACT | |
| | TAGAAAATTCTTGGCAATCTATCTCAGGTCCATGTATGTTAACTCAT | |
| | ATATTCTTTCGTGTAACAGATAGCTTTACTAAAGAAACTGTTGATTC | |
| | TCTTTACAAATATCATGATTTAGTTAGATGGTCATCATTCGTGCTTCG | |
| | TCTTGCTGACGACTTAGGTACAAGCGTTGAAGAAGTATCTCGTGGTG | |
| | ATGTGCCAAAATCTTTACAATGCTACATGAGTGATTATAACGCTAGT | |
| | GAGGCTGAAGCACGTAAACACGTAAAATGGTTAATTGCAGAAGTAT | |
| | GGAAAAAGATGAATGCAGAACGTGTTTCTAAAGATAGTCCTTTTGGT | |
| | AAAGATTTTATAGGTTGTGCTGTTGATTTAGGTCGTATGGCTCAATT | |
| | AATGTATCACAATGGAGATGGTCATGGTACTCAACACCCTATTATTC | |
| | ATCAACAAATGACACGTACTTTATTTGAACCATTCGCTGGTACCGGT | |
| | GAAAACTTATACTTTCAAGGCTCAGGTGGCGGTGGAAGTGATTACA | |
| | AAGATGATGATGATAAAGGAACCGGTTAA | |
| 75 | ATGGTACCAAGACGTACTGGTGGCTATCAACCTACACTTTGGGATTT | Cineole (S. officinalis) |
| | TTCAACAATTCAATTATTTGATAGTGAATATAAAGAAGAAAAACATC | |
| | TTATGCGTGCTGCTGGTATGATTGCTCAAGTGAACATGTTACTTCAA | |
| | GAAGAAGTAGACAGCATCCAACGTCTTGAATTAATTGATGACTTAC | |
| | GTCGTTTAGGTATATCTTGCCACTTTGATCGTGAAATTGTAGAGATTT | |
| | TAAACAGTAAATACTACACCAACAATGAAATTGATGAATCAGATTT | |
| | ATACAGTACAGCACTTAGATTCAAACTTTTACGTCAATATGATTTTA | |
| | GCGTTAGCCAAGAAGTTTTTGATTGTTTTAAAAATGACAAAGGTACA | |
| | GATTTCAAACCATCATTAGTTGACGATACACGTGGCTTATTACAATT | |
| | ATATGAAGCATCATTTTTATCAGCTCAGGGTGAAGAAACTTTACATT | |
| | TAGCACGTGATTTTGCTACTAAATTCTTACATAAAAGAGTTTTAGTA | |
| | GATAAAGATATCAATTTATTATCTAGTATCGAGCGTGCTTTAGAATT | |
| | ACCAACACACTGGCGTGTACAAATGCCTAACGCTAGATCATTCATCG | |
| | ACGCATATAAAAGAAGACCAGACATGAACCCTACAGTATTAGAGTT | |
| | AGCAAAACTTGACTTTAACATGGTTCAAGCACAGTTCCAACAAGAA | |
| | TTAAAAGAAGCCAGTCGCTGGTGGAACTCTACAGGATTAGTACATG | |
| | AATTACCATTTGTACGTGATCGTATTGTGGAATGTTATTATTGGACT | |
| | ACTGGTGTAGTAGAACGTCGTGAACACGGTTACGAACGTATTATGTT | |
| | AACAAAAATTAACGCTTTAGTTACAACAATCGATGATGTTTTTGACA | |
| | TTTATGGTACTTTAGAAGAATTACAACTTTTTACAACTGCTATTCAA | |
| | AGATGGGACATTGAGTCTATGAAACAACTTCCACCCTATATGCAAAT | |
| | CTGCTACTTAGCTTTATTCAACTTCGTAAATGAGATGGCTTACGATA | |
| | CATTACGTGATAAAGGTTTTAATAGTACTCCATATTTACGCAAAGCC | |
| | TGGGTAGACTTAGTAGAAAGCTACTTAATTGAAGCTAAATGGTATTA | |
| | TATGGGTCACAAACCAAGTTTAGAAGAGTACATGAAAAACTCATGG | |
| | ATTTCTATCGGAGGTATTCCAATTTTATCACATTTATTCTTTCGTTTA | |
| | ACAGACAGTATCGAAGAAGAAGACGCTGAATCAATGCATAAATATC | |
| | ACGATATAGTACGTGCCTCTTGTACTATTTTACGTTTAGCTGATGATA | |
| | TGGGTACATCATTAGATGAAGTTGAACGTGGCGATGTTCCTAAATCT | |
| | GTACAATGCTATATGAATGAGAAAAACGCCTCTGAAGAAGAAGCAC | |
| | GTGAACATGTTCGTAGTTTAATTGATCAGACATGGAAGATGATGAAT | |
| | AAAGAAATGATGACTTCATCATTTTCAAAATACTTCGTACAAGTGTC | |
| | TGCAAATCTTGCTCGTATGGCACAATGGATATATCAACATGAAAGTG | |
| | ATGGTTTCGGTATGCAACACTCTTTAGTTAACAAAATGCTTCGTGGT | |
| | TTACTTTTTGACCGTTATGAAGGTACCGGTGAAAACTTATACTTTCA | |
| | AGGCTCAGGTGGCGGTGGAAGTGATTACAAAGATGATGATGATAAA | |
| | GGAACCGGTTAA | |
| 76 | ATGGTACCACGTCGCATGGGTGATTTTCATTCAAACTTATGGGATGA | Pinene (A. grandis) |
| | TGATGTAATTCAATCTTTACCCACAGCTTACGAAGAAAAATCTTATC | |
| | TTGAACGTGCTGAGAAGTTAATTGGAGAAGTTGAAAATATGTTCAA | |
| | CAGTATGAGTTTAGAAGATGGTGAACTTATGAGTCCATTAAATGATT | |
| | TAATTCAACGCCTTTGGATTGTTGATTCTTTAGGTAGATTAGGTATCC | |
| | ATCGTCACTTTAAAGATGAGATTAAAAGTGCTTTAGATTATGTTTAC | |
| | AGTTACTGGGGTGAAAACGGAATAGGTTGTGGTCGTGAAAGTGCTG | |
| | TAACTGATTTAAACAGTACAGCTTTAGGCTTTCGTACACTTCGTTTAC | |
| | ACGGTTATCCAGTTTCATCTGATGTATTTAAAGCATTTAAAGGTCAA | |
| | AATGGTCAATTCAGTTGTTCAGAAAATATCCAAACAGATGAAGAAA | |
| | TTCGTGGTGTTCTTAACTTATTTAGAGCCAGTTTAATAGCCTTCCCTG | |
| | GTGAGAAAATAATGGACGAAGCTGAAATCTTCTCTACAAAATACTT | |
| | AAAGGAAGCATTACAAAAGATCCCAGTTAGTTCATTATCACGTGAA | |
| | ATCGGTGATGTACTTGAATATGGATGGCATACATACTTACCACGTTT | |
| | AGAAGCACGTAACTATATTCATGTTTTCGGACAAGATACAGAGAAT | |
| | ACAAAAAGTTATGTAAAATCAAAGAAACTTTTAGAATTAGCTAAAT | |
| | TAGAATTTAACATTTTTCAGAGCTTACAAAAACGTGAATTAGAAAGC | |
| | CTTGTTCGTTGGTGGAAAGAATCTGGATTTCCTGAAATGACATTCTG | |
| | TAGACACAGACACGTGGAATATTACACACTTGCATCATGTATTGCAT | |
| | TCGAACCTCAGCATAGTGGTTTTCGTTTAGGTTTTGCTAAAACATGT | |
| | CACCTTATAACAGTTTTAGATGACATGTATGACACTTTCGGCACCGT | |
| | AGACGAATTAGAGTTATTTACAGCAACTATGAAACGTTGGGACCCA | |
| | AGTTCAATTGACTGCCTTCCAGAATACATGAAAGGAGTTTACATTGC | |
| | TGTGTATGATACAGTTAATGAAATGGCTCGTGAAGCTGAGGAAGCT | |
| | CAAGGTCGCGATACACTTACATACGCTCGTGAGGCCTGGGAGGCTT | |
| | ATATAGATTCTTATATGCAAGAAGCTCGCTGGATTGCTACTGGATAC | |
| | TTACCTTCTTTCGATGAATATTATGAAAATGGTAAGGTTTCATGTGG | |
| | TCACCGTATATCTGCTTTACAACCAATTCTTACTATGGATATTCCATT | |
| | TCCAGATCACATTTTAAAGGAAGTTGACTTTCCTTCTAAACTTAATG | |
| | ACTTAGCTTGTGCTATCTTACGCCTTCGCGGTGATACTCGTTGTTACA | |
| | AAGCAGACCGTGCACGTGGTGAAGAGGCTAGTTCTATTTCTTGTTAT | |
| | ATGAAAGATAATCCAGGTGTTTCTGAAGAAGATGCCTTAGATCATAT | |
| | TAACGCAATGATCAGTGATGTTATTAAGGGCTTAAACTGGGAATTAC | |
| | TTAAACCCGACATTAACGTACCTATTTCTGCTAAGAAACATGCTTTC | |
| | GACATTGCTCGTGCTTTTCACTACGGTTATAAATATCGTGATGGCTA | |
| | TTCAGTTGCTAATGTTGAAACAAAATCTTTAGTTACACGTACTTTACT | |
| | TGAATCAGTTCCATTAGGTACCGGTGAAAACTTATACTTTCAAGGCT | |
| | CAGGTGGCGGTGGAAGTGATTACAAAGATGATGATGATAAAGGAAC | |
| | CGGTTAA | |
| 77 | ATGGTACCACGTAGAGTTGGTAATTATCATTCTAATCTTTGGGATGA | Camphene |
| | TGATTTTATACAAAGTTTAATTTCTACACCTTACGGTGCTCCTGACTA | (A. grandis) |
| | CCGTGAACGCGCTGATCGTCTTATTGGTGAAGTAAAAGATATTATGT | |
| | TTAATTTCAAATCTTTAGAGGATGGTGGTAATGACTTATTACAACGT | |
| | TTACTTTTAGTTGATGACGTAGAACGTTTAGGCATTGATCGTCATTTC | |
| | AAAAAGGAAATTAAGACTGCATTAGATTATGTAAATAGTTATTGGA | |
| | ATGAAAAAGGAATTGGTTGTGGTCGTGAGTCTGTAGTTACAGACTTA | |
| | AATTCAACTGCTTTAGGCCTTCGTACCTTAAGATTACATGGTTATACT | |
| | GTTAGCTCTGACGTTTTAAATGTTTTTAAAGATAAAAATGGTCAATT | |
| | TTCATCTACAGCTAATATTCAAATTGAAGGTGAAATTCGTGGTGTTT | |
| | TAAATCTTTTTCGTGCCTCTCTTGTAGCTTTTCCAGGTGAGAAAGTGA | |
| | TGGATGAGGCTGAAACTTTTTCAACAAAATATCTTCGTGAAGCATTA | |
| | CAGAAAATTCCTGCCAGTTCAATTTTATCATTAGAAATACGTGATGT | |
| | ATTAGAATATGGATGGCATACTAATTTACCACGTTTAGAAGCACGTA | |
| | ATTACATGGATGTTTTCGGTCAGCACACCAAGAACAAAAATGCAGC | |
| | CGAAAAATTACTTGAATTAGCAAAATTAGAGTTCAATATCTTTCACA | |
| | GCTTACAAGAACGTGAATTAAAGCACGTTTCAAGATGGTGGAAAGA | |
| | CTCTGGTAGTCCAGAGATGACTTTCTGTCGCCACCGCCATGTGGAAT | |
| | ATTATGCTTTAGCTTCTTGTATTGCTTTCGAACCCCAGCACAGTGGTT | |
| | TCCGTTTAGGTTTTACTAAAATGAGTCATTTAATCACAGTGTTAGAT | |
| | GATATGTATGATGTATTCGGTACAGTTGATGAATTAGAGTTATTTAC | |
| | CGCCACTATTAAACGTTGGGACCCTTCTGCTATGGAATGTTTACCAG | |
| | AGTACATGAAAGGTGTTTACATGATGGTTTATCATACAGTTAACGAA | |
| | ATGGCTCGTGTGGCAGAAAAGGCTCAAGGTAGAGACACATTAAACT | |
| | ATGCTCGTCAAGCCTGGGAAGCATGTTTTGACTCTTATATGCAAGAA | |
| | GCAAAATGGATTGCAACAGGTTACTTACCTACATTCGAGGAATATTT | |
| | AGAAAATGGTAAAGTGAGTTCAGCACATCGTCCTTGTGCATTACAAC | |
| | CTATTTTAACTCTTGATATTCCATTTCCCGATCATATTCTTAAAGAAG | |
| | TGGATTTCCCAAGCAAACTTAATGACTTAATTTGTATTATCTTACGTC | |
| | TTAGAGGAGACACACGTTGCTATAAAGCAGACCGTGCCCGTGGTGA | |
| | AGAAGCATCATCAATATCTTGTTATATGAAAGATAACCCAGGTTTAA | |
| | CTGAAGAAGATGCTTTAAACCACATTAACTTTATGATTCGTGACGCA | |
| | ATCCGCGAATTAAACTGGGAGTTACTTAAACCAGATAATAGTGTTCC | |
| | AATTACTTCAAAGAAACATGCTTTTGATATTTCACGTGTGTGGCACC | |
| | ACGGATACCGTTATCGTGATGGTTACAGCTTTGCAAACGTGGAAACT | |
| | AAAAGTCTTGTAATGCGTACTGTAATAGAACCAGTACCATTAGGTAC | |
| | CGGTGAAAACTTATACTTTCAAGGCTCAGGTGGCGGTGGAAGTGATT | |
| | ACAAAGATGATGATGATAAAGGAACCGGTTAA | |
| 78 | ATGGTACCACGTCGTTCAGGAGATTATCAACCAAGTTTATGGGACTT | Sabinene (S. officinalis) |
| | TAATTACATTCAATCTTTAAACACACCTTACAAAGAACAACGTCATT | |
| | TTAATCGTCAAGCTGAGTTAATTATGCAAGTTCGTATGTTATTAAAG | |
| | GTAAAAATGGAAGCAATTCAACAATTAGAGTTAATAGATGATTTAC | |
| | AGTACTTAGGATTATCATATTTCTTTCAAGACGAAATTAAACAAATC | |
| | TTAAGCTCTATTCACAATGAACCTCGTTATTTTCATAATAATGACCTT | |
| | TATTTCACTGCTTTAGGTTTTAGAATTTTACGTCAACATGGTTTTAAT | |
| | GTTTCAGAAGACGTATTTGACTGCTTTAAAATCGAAAAATGTTCTGA | |
| | CTTTAATGCTAACTTAGCTCAGGACACAAAGGGTATGTTACAATTAT | |
| | ATGAAGCTAGTTTCTTATTAAGAGAAGGAGAAGATACACTTGAATT | |
| | AGCTCGTCGTTTTAGTACACGTTCTTTACGTGAAAAATTTGATGAAG | |
| | GTGGTGACGAGATAGATGAAGATTTAAGTAGTTGGATTCGTCATTCT | |
| | TTAGATTTACCATTACACTGGCGTGTTCAAGGTTTAGAAGCTCGTTG | |
| | GTTTTTAGATGCCTATGCTCGTCGTCCAGATATGAACCCTCTTATTTT | |
| | CAAATTAGCTAAATTAAATTTTAACATTGTTCAAGCTACATACCAAG | |
| | AAGAATTAAAAGACATCTCTCGTTGGTGGAACAGTAGTTGTTTAGCA | |
| | GAGAAATTACCCTTCGTTCGCGATCGTATTGTAGAATGTTTCTTCTG | |
| | GGCTATTGCTGCTTTCGAACCACACCAATACTCATATCAACGTAAAA | |
| | TGGCCGCTGTAATTATTACATTTATTACTATTATTGATGATGTTTACG | |
| | ATGTATATGGTACTATTGAAGAATTAGAGTTATTAACAGATATGATT | |
| | CGTAGATGGGATAATAAGAGTATTAGTCAACTTCCTTACTATATGCA | |
| | AGTTTGTTATTTAGCTCTTTATAACTTCGTAAGTGAACGCGCATACG | |
| | ACATCTTAAAAGATCAACACTTTAACAGTATTCCATACCTTCAAAGA | |
| | AGTTGGGTTTCATTAGTTGAGGGATACTTAAAAGAAGCATATTGGTA | |
| | CTATAACGGTTACAAACCAAGTCTTGAAGAATATCTTAATAATGCAA | |
| | AAATTAGTATTAGTGCACCCACCATTATTTCACAATTATACTTTACTT | |
| | TAGCAAATAGTATCGACGAAACTGCCATTGAAAGTTTATACCAATAT | |
| | CACAACATTTTATACTTATCAGGTACTATCTTACGTTTAGCTGATGAT | |
| | TTAGGAACTTCACAACATGAATTAGAACGTGGTGATGTTCCCAAAGC | |
| | TATTCAATGTTATATGAATGATACAAATGCATCAGAAAGAGAAGCT | |
| | GTAGAACATGTTAAATTTCTTATTCGTGAAGCCTGGAAAGAAATGAA | |
| | TACAGTTACTACCGCATCAGATTGTCCTTTTACAGACGATCTTGTTGC | |
| | CGCCGCAGCTAATTTAGCTCGTGCTGCTCAATTCATTTACTTAGATG | |
| | GTGATGGTCATGGTGTACAACATAGCGAAATTCATCAGCAAATGGG | |
| | CGGTCTTCTTTTTCAACCATACGTTGGTACCGGTGAAAACTTATACTT | |
| | TCAAGGCTCAGGTGGCGGTGGAAGTGATTACAAAGATGATGATGAT | |
| | AAAGGAACCGGTTAA | |
| 79 | ATGGTACCACGCAGAATTGGTGATTACCATAGTAACATTTGGGATGA | Myrcene (A. grandis) |
| | TGATTTTATCCAGTCACTTTCTACCCCTTATGGTGAACCATCTTACCA | |
| | AGAAAGAGCTGAACGTCTTATTGTAGAAGTGAAAAAGATTTTCAAC | |
| | AGTATGTACTTAGATGACGGTCGTTTAATGAGTTCTTTTAATGACTT | |
| | AATGCAACGTTTATGGATTGTAGACTCAGTAGAACGTTTAGGTATTG | |
| | CCCGTCACTTCAAAAATGAAATTACATCTGCCCTTGACTATGTTTTTC | |
| | GTTATTGGGAAGAAAACGGTATAGGTTGTGGTCGTGATTCTATTGTA | |
| | ACTGACTTAAATAGCACAGCTTTAGGTTTTCGTACACTTCGTTTACA | |
| | CGGTTATACAGTTTCTCCAGAGGTTTTAAAAGCATTTCAAGATCAAA | |
| | ATGGTCAATTCGTTTGTTCACCAGGACAAACAGAAGGTGAAATTCGT | |
| | TCAGTTTTAAATTTATATCGTGCAAGTTTAATTGCCTTTCCAGGCGAA | |
| | AAAGTTATGGAAGAAGCAGAAATCTTCTCTACTCGCTATTTAAAAGA | |
| | AGCTCTTCAAAAGATTCCAGTTAGCGCATTATCACAAGAAATCAAAT | |
| | TTGTTATGGAATATGGATGGCATACAAATTTACCTAGATTAGAAGCA | |
| | CGTAACTATATTGATACTTTAGAAAAGGATACATCAGCTTGGTTAAA | |
| | CAAAAATGCAGGTAAAAAGTTATTAGAATTAGCTAAATTAGAATTT | |
| | AACATCTTTAACTCATTACAACAAAAAGAATTACAATACTTACTTCG | |
| | CTGGTGGAAAGAATCTGACTTACCTAAATTAACCTTTGCACGTCATA | |
| | GACACGTTGAATTTTACACATTAGCTTCTTGTATTGCTATTGATCCCA | |
| | AACATTCAGCATTCCGTTTAGGATTCGCTAAAATGTGTCACTTAGTT | |
| | ACAGTTCTTGACGATATTTATGATACTTTCGGTACTATTGATGAACTT | |
| | GAGTTATTTACTTCTGCAATTAAACGTTGGAATAGTTCTGAAATTGA | |
| | ACACTTACCAGAATATATGAAATGCGTGTATATGGTTGTTTTTGAAA | |
| | CTGTTAATGAATTAACTCGTGAAGCTGAGAAAACACAAGGACGTAA | |
| | CACTTTAAACTATGTTCGTAAAGCATGGGAAGCATATTTTGATTCTT | |
| | ATATGGAGGAAGCAAAGTGGATCTCAAACGGATATTTACCAATGTT | |
| | TGAAGAATACCACGAAAATGGTAAAGTGTCATCTGCATACCGTGTA | |
| | GCAACATTACAACCAATTTTAACTTTAAACGCTTGGTTACCCGACTA | |
| | CATTCTTAAAGGAATTGATTTCCCAAGTCGTTTTAACGATTTAGCTA | |
| | GTTCATTCTTACGTTTACGTGGCGATACTCGCTGTTACAAAGCTGAC | |
| | CGTGATCGTGGTGAAGAAGCTAGCTGCATTTCTTGTTACATGAAAGA | |
| | TAATCCAGGTTCTACCGAAGAAGATGCACTTAATCACATTAACGCTA | |
| | TGGTAAATGACATCATTAAAGAATTAAACTGGGAATTATTACGCAGT | |
| | AATGATAATATTCCTATGTTAGCTAAAAAGCACGCTTTTGATATTAC | |
| | TCGTGCACTTCACCACTTATACATTTATCGCGATGGTTTCAGTGTTGC | |
| | TAATAAAGAAACTAAAAAGTTAGTTATGGAGACATTACTTGAATCA | |
| | ATGTTATTTGGTACCGGTGAAAACTTATACTTTCAAGGCTCAGGTGG | |
| | CGGTGGAAGTGATTACAAAGATGATGATGATAAAGGAACCGGTTAA | |
| 80 | ATGGTACCACAATCTGCTGAAAAGAACGACTCTTTATCAAGTTCTAC | Abietadiene |
| | ATTAGTTAAGAGAGAATTTCCACCCGGTTTCTGGAAAGACGACTTAA | (A. grandis) |
| | TCGACAGTTTAACTTCAAGTCACAAAGTAGCTGCTAGCGATGAAAA | |
| | ACGTATCGAAACCTTAATTTCAGAAATTAAGAATATGTTTCGTTGTA | |
| | TGGGTTATGGTGAGACAAATCCATCAGCTTATGATACTGCTTGGGTA | |
| | GCTCGCATCCCAGCAGTTGATGGATCAGATAATCCTCACTTTCCAGA | |
| | GACTGTGGAATGGATCTTACAAAATCAATTAAAAGATGGTTCTTGGG | |
| | GTGAAGGTTTTTACTTCCTTGCTTATGATCGCATTTTAGCCACTTTAG | |
| | CTTGTATTATCACACTTACACTTTGGCGTACTGGAGAAACACAAGTA | |
| | CAGAAAGGTATCGAATTTTTCCGCACTCAAGCAGGTAAAATGGAAG | |
| | ATGAAGCAGATTCACACCGTCCAAGTGGTTTTGAGATTGTATTTCCT | |
| | GCTATGTTAAAAGAGGCTAAGATTTTAGGCTTAGATTTACCTTATGA | |
| | TCTTCCTTTTCTTAAACAAATTATTGAAAAGAGAGAAGCTAAGTTAA | |
| | AACGTATTCCTACAGATGTTTTATATGCTTTACCAACTACTTTACTTT | |
| | ATTCATTAGAAGGTTTACAAGAAATAGTAGACTGGCAAAAAATCAT | |
| | GAAATTACAAAGTAAAGATGGTAGTTTCTTATCTTCTCCTGCCTCAA | |
| | CAGCAGCAGTATTTATGAGAACAGGTAACAAAAAGTGTTTAGATTT | |
| | CTTAAATTTCGTGCTTAAAAAGTTCGGTAATCATGTTCCATGCCACT | |
| | ATCCTTTAGACCTTTTTGAGCGTCTTTGGGCAGTTGATACTGTTGAAA | |
| | GATTAGGTATTGACCGTCATTTTAAAGAAGAAATAAAAGAGGCTTT | |
| | AGACTATGTGTATTCACACTGGGACGAACGTGGTATTGGTTGGGCTC | |
| | GTGAAAACCCCGTTCCAGATATTGACGATACAGCAATGGGTCTTCGT | |
| | ATTTTACGTCTTCATGGTTACAATGTTAGCAGCGATGTTCTTAAAAC | |
| | ATTTCGTGATGAAAATGGTGAGTTCTTTTGCTTTTTAGGACAAACAC | |
| | AAAGAGGTGTGACTGATATGTTAAATGTTAATCGTTGTAGCCATGTA | |
| | TCTTTCCCTGGTGAAACTATAATGGAAGAGGCAAAATTATGTACTGA | |
| | ACGTTACTTACGCAACGCATTAGAAAATGTAGACGCTTTTGATAAGT | |
| | GGGCATTTAAGAAAAACATTCGTGGTGAGGTAGAATATGCTCTTAA | |
| | ATATCCTTGGCATAAATCAATGCCACGTTTAGAAGCACGTTCATATA | |
| | TTGAAAATTACGGTCCAGATGATGTTTGGTTAGGTAAAACTGTTTAT | |
| | ATGATGCCTTACATTTCAAATGAAAAGTACTTAGAGTTAGCTAAACT | |
| | TGATTTTAACAAAGTTCAGTCAATCCACCAGACAGAACTTCAAGACT | |
| | TACGCCGTTGGTGGAAAAGTTCTGGTTTTACAGATTTAAACTTTACA | |
| | AGAGAACGTGTTACTGAAATTTACTTTTCACCTGCATCTTTTATCTTC | |
| | GAACCAGAATTTAGTAAATGTCGTGAGGTTTATACAAAAACTTCTAA | |
| | TTTTACTGTAATTTTAGACGATTTATATGACGCTCATGGCTCTTTAGA | |
| | TGACTTAAAACTTTTTACAGAGAGTGTTAAACGTTGGGATTTATCTT | |
| | TAGTTGACCAAATGCCCCAGCAGATGAAAATCTGTTTTGTAGGTTTC | |
| | TATAATACATTCAACGATATTGCTAAAGAAGGTAGAGAACGTCAAG | |
| | GTCGTGATGTTTTAGGTTATATTCAAAACGTATGGAAAGTACAACTT | |
| | GAAGCATATACTAAAGAAGCAGAATGGTCAGAAGCAAAATATGTTC | |
| | CTAGTTTTAACGAATACATTGAAAATGCTTCAGTTTCAATTGCCTTA | |
| | GGTACAGTAGTACTTATCAGTGCTTTATTTACCGGAGAAGTTTTAAC | |
| | AGATGAAGTTTTATCTAAAATTGACCGTGAAAGTAGATTCTTACAGT | |
| | TAATGGGCTTAACTGGACGTTTAGTAAATGATACTAAAACATATCAA | |
| | GCTGAGCGTGGTCAAGGTGAAGTTGCTAGTGCAATTCAATGTTATAT | |
| | GAAAGACCACCCTAAAATTAGTGAAGAAGAAGCATTACAACATGTA | |
| | TATTCTGTAATGGAAAATGCATTAGAAGAATTAAATCGTGAGTTCGT | |
| | TAACAACAAAATTCCAGACATCTATAAACGTCTTGTTTTCGAAACTG | |
| | CACGTATAATGCAATTATTTTACATGCAAGGTGATGGTTTAACATTA | |
| | AGTCACGATATGGAAATTAAAGAGCACGTAAAGAATTGTTTATTCC | |
| | AGCCAGTAGCTGGTACCGGTGAAAACTTATACTTTCAAGGCTCAGGT | |
| | GGCGGTGGAAGTGATTACAAAGATGATGATGATAAAGGAACCGGTT | |
| | AA | |
| 81 | ATGGTACCATCTTCATCAACAGGCACTTCAAAAGTAGTAAGCGAAA | Taxadiene |
| | CATCTTCAACTATTGTAGACGATATTCCACGTCTTTCAGCAAATTATC | (T. brevifolia) |
| | ATGGTGATTTATGGCATCACAACGTAATTCAGACTTTAGAAACACCA | |
| | TTTAGAGAAAGTTCAACTTATCAAGAGCGTGCAGATGAATTAGTAGT | |
| | GAAAATCAAAGATATGTTCAATGCATTAGGTGACGGTGACATCTCA | |
| | CCTTCAGCTTATGATACTGCATGGGTAGCTCGTGTTGCTACCATTTCT | |
| | TCTGATGGTAGCGAAAAACCACGTTTTCCTCAAGCTCTTAATTGGGT | |
| | TTTTAACAATCAATTACAAGATGGATCATGGGGTATTGAATCACATT | |
| | TTAGTTTATGCGATCGTTTACTTAATACTACAAATTCAGTTATTGCTT | |
| | TATCAGTATGGAAAACTGGTCACTCACAGGTTCAACAAGGTGCCGA | |
| | ATTTATTGCTGAAAATTTACGTCTTTTAAATGAAGAAGACGAATTAA | |
| | GTCCTGATTTTCAAATTATCTTCCCAGCTTTATTACAGAAAGCCAAG | |
| | GCTTTAGGAATCAATTTACCCTATGATTTACCATTCATCAAATATCTT | |
| | AGTACAACACGCGAAGCTCGTTTAACAGATGTGTCAGCTGCTGCTGA | |
| | CAACATACCAGCCAATATGCTTAATGCACTTGAAGGTTTAGAAGAA | |
| | GTGATTGATTGGAATAAAATCATGCGTTTTCAATCTAAAGATGGTTC | |
| | ATTTTTATCTTCTCCAGCTAGTACAGCCTGTGTTTTAATGAATACAGG | |
| | TGATGAAAAATGTTTCACATTCTTAAATAACTTATTAGATAAATTCG | |
| | GCGGTTGTGTTCCATGTATGTATAGCATTGATTTATTAGAACGTTTAT | |
| | CTTTAGTGGACAACATTGAACACTTAGGTATTGGTCGTCACTTTAAA | |
| | CAAGAAATCAAAGGTGCATTAGATTATGTATATCGTCATTGGTCTGA | |
| | ACGCGGTATCGGTTGGGGTAGAGACTCTTTAGTTCCAGATTTAAACA | |
| | CCACAGCTTTAGGTTTACGCACATTAAGAATGCACGGTTATAACGTG | |
| | TCTAGTGATGTACTTAACAATTTCAAAGACGAAAATGGTCGTTTCTT | |
| | TAGTAGTGCTGGTCAAACACACGTAGAGTTACGTTCTGTTGTAAATC | |
| | TTTTTCGCGCCTCAGATTTAGCCTTTCCAGACGAACGTGCAATGGAT | |
| | GATGCTCGTAAATTCGCAGAACCATATTTACGTGAAGCATTAGCTAC | |
| | AAAAATATCAACAAATACAAAGTTATTCAAAGAAATTGAATATGTT | |
| | GTTGAATACCCTTGGCACATGTCAATTCCACGTTTAGAAGCTCGTAG | |
| | TTATATTGACAGTTATGATGATAATTATGTATGGCAACGTAAGACTT | |
| | TATATCGTATGCCATCATTAAGTAATTCAAAATGTTTAGAACTTGCT | |
| | AAATTAGATTTCAATATTGTTCAATCTTTACACCAAGAAGAACTTAA | |
| | ACTTTTAACTCGTTGGTGGAAAGAATCTGGTATGGCAGACATAAATT | |
| | TCACCCGCCATCGTGTAGCTGAAGTTTACTTTTCTAGTGCTACATTTG | |
| | AGCCAGAATATAGTGCTACTCGTATTGCATTCACAAAAATTGGTTGC | |
| | TTACAAGTACTTTTCGATGATATGGCTGACATTTTCGCCACTTTAGAT | |
| | GAGTTAAAAAGTTTTACTGAAGGTGTTAAACGCTGGGACACATCATT | |
| | ATTACATGAAATTCCCGAATGTATGCAAACTTGTTTTAAAGTATGGT | |
| | TTAAACTTATGGAAGAAGTAAACAACGACGTAGTAAAAGTTCAAGG | |
| | AAGAGATATGTTAGCACATATTCGTAAACCCTGGGAATTATACTTTA | |
| | ATTGTTATGTTCAAGAACGTGAATGGTTAGAAGCTGGTTATATTCCT | |
| | ACATTCGAAGAATATCTTAAAACTTATGCTATTAGTGTAGGCCTTGG | |
| | TCCTTGTACCTTACAACCTATTCTTTTAATGGGTGAGTTAGTTAAAGA | |
| | TGATGTAGTAGAAAAAGTTCATTACCCTTCTAACATGTTCGAATTAG | |
| | TTTCTTTAAGCTGGCGTTTAACTAATGATACCAAAACATATCAAGCA | |
| | GAAAAAGTACGCGGTCAACAAGCTAGTGGCATTGCCTGTTATATGA | |
| | AAGACAATCCAGGTGCTACTGAAGAAGATGCTATTAAACACATTTG | |
| | TCGTGTTGTTGATCGTGCATTAAAAGAAGCAAGTTTCGAATATTTCA | |
| | AGCCTTCAAATGACATTCCTATGGGTTGTAAATCTTTTATCTTTAACT | |
| | TACGTTTATGTGTACAAATTTTCTATAAATTCATTGATGGTTATGGTA | |
| | TCGCAAACGAAGAAATTAAGGACTACATTCGTAAGGTTTATATTGAT | |
| | CCAATTCAAGTTGGTACCGGTGAAAACTTATACTTTCAAGGCTCAGG | |
| | TGGCGGTGGAAGTGATTACAAAGATGATGATGATAAAGGAACCGGT | |
| | TAA | |
| 82 | ATGGTACCACACAAGTTCACAGGTGTTAACGCTAAATTCCAGCAACC | FPP (G. gallus) |
| | AGCATTAAGAAATTTATCTCCAGTGGTAGTTGAGCGCGAACGTGAG | |
| | GAATTTGTAGGATTCTTTCCACAAATTGTTCGTGACTTAACTGAAGA | |
| | TGGTATTGGTCATCCAGAAGTAGGTGACGCTGTAGCTCGTCTTAAAG | |
| | AAGTATTACAATACAACGCACCTGGTGGTAAATGCAATAGAGGTTT | |
| | AACAGTTGTTGCAGCTTACCGTGAACTTTCTGGACCAGGTCAAAAAG | |
| | ACGCTGAAAGTCTTCGTTGTGCTTTAGCAGTAGGATGGTGTATTGAA | |
| | TTATTCCAAGCCTTTTTCTTAGTTGCTGACGATATAATGGACCAGTCA | |
| | TTAACTAGACGTGGTCAATTATGTTGGTACAAGAAAGAAGGTGTTG | |
| | GTTTAGATGCAATAAATGATTCTTTTCTTTTAGAAAGCTCTGTGTATC | |
| | GCGTTCTTAAAAAGTATTGCCGTCAACGTCCATATTATGTACATTTA | |
| | TTAGAGCTTTTTCTTCAAACAGCTTACCAAACAGAATTAGGACAAAT | |
| | GTTAGATTTAATCACTGCTCCTGTATCTAAGGTAGATTTAAGCCATTT | |
| | CTCAGAAGAACGTTACAAAGCTATTGTTAAGTATAAAACTGCTTTCT | |
| | ATTCATTCTATTTACCAGTTGCAGCAGCTATGTATATGGTTGGTATA | |
| | GATTCTAAAGAAGAACATGAAAACGCAAAAGCTATTTTACTTGAGA | |
| | TGGGTGAATACTTCCAAATTCAAGATGATTATTTAGATTGTTTTGGC | |
| | GATCCTGCTTTAACAGGTAAAGTAGGTACTGATATTCAAGATAACAA | |
| | ATGTTCATGGTTAGTTGTGCAATGCTTACAAAGAGTAACACCAGAAC | |
| | AACGTCAACTTTTAGAAGATAATTACGGTCGTAAAGAACCAGAAAA | |
| | AGTTGCTAAAGTTAAAGAATTATATGAGGCTGTAGGTATGAGAGCC | |
| | GCCTTTCAACAATACGAAGAAAGTAGTTACCGTCGTCTTCAAGAGTT | |
| | AATTGAGAAACATTCTAATCGTTTACCAAAAGAAATTTTCTTAGGTT | |
| | TAGCTCAGAAAATATACAAACGTCAAAAAGGTACCGGTGAAAACTT | |
| | ATACTTTCAAGGCTCAGGTGGCGGTGGAAGTGATTACAAAGATGAT | |
| | GATGATAAAGGAACCGGTTAA | |
| 83 | ATGGTACCATCATTAACTGAAGAAAAACCAATTCGCCCAATCGCAA | Amorphadiene |
| | ACTTTCCTCCAAGCATTTGGGGAGATCAATTCTTAATTTACGAAAAA | (A. annua) |
| | CAAGTAGAACAAGGTGTTGAACAGATTGTTAACGACCTTAAGAAAG | |
| | AAGTGCGCCAACTTTTAAAAGAGGCTTTAGATATTCCAATGAAACAC | |
| | GCAAACCTTTTAAAACTTATTGACGAAATTCAACGTCTTGGTATTCC | |
| | ATATCACTTTGAACGTGAAATTGATCATGCATTACAATGTATCTATG | |
| | AAACTTATGGTGATAATTGGAATGGTGATCGTTCTTCATTATGGTTC | |
| | CGTTTAATGCGTAAACAAGGTTATTATGTTACATGTGACGTGTTTAA | |
| | CAATTACAAAGATAAAAATGGTGCATTTAAACAATCTTTAGCTAATG | |
| | ATGTTGAAGGTTTATTAGAATTATATGAAGCTACTTCAATGCGTGTT | |
| | CCAGGTGAAATTATTCTTGAAGATGCATTAGGTTTTACACGTTCTCG | |
| | TTTATCTATTATGACAAAAGACGCATTTAGTACAAATCCTGCTTTATT | |
| | TACTGAAATTCAGCGTGCCCTTAAACAGCCTTTATGGAAACGTTTAC | |
| | CAAGAATTGAAGCTGCTCAATATATTCCATTTTATCAACAACAAGAT | |
| | TCTCACAATAAGACATTACTTAAATTAGCCAAATTAGAATTTAATCT | |
| | TTTACAATCATTACATAAAGAAGAATTAAGTCATGTGTGTAAATGGT | |
| | GGAAAGCATTTGATATTAAGAAGAATGCTCCATGTTTACGTGATAGA | |
| | ATTGTAGAGTGTTACTTTTGGGGCCTTGGTAGTGGTTACGAGCCACA | |
| | ATATTCACGTGCTCGTGTATTCTTTACAAAAGCTGTTGCAGTTATTAC | |
| | TTTAATTGACGATACCTATGATGCATACGGAACCTATGAGGAGCTTA | |
| | AAATTTTCACTGAAGCTGTAGAACGTTGGTCTATAACTTGTTTAGAT | |
| | ACTTTACCAGAATATATGAAACCCATCTACAAATTATTCATGGACAC | |
| | ATACACTGAAATGGAAGAATTTTTAGCAAAAGAAGGTCGCACAGAC | |
| | CTTTTTAACTGTGGTAAAGAATTTGTTAAAGAGTTTGTTCGTAACTTA | |
| | ATGGTAGAAGCTAAGTGGGCTAATGAAGGTCACATTCCTACTACAG | |
| | AAGAGCACGATCCAGTAGTAATAATTACAGGTGGAGCAAACTTACT | |
| | TACCACAACTTGTTACTTAGGTATGTCTGACATTTTTACAAAAGAAT | |
| | CAGTAGAGTGGGCAGTATCTGCACCACCATTATTCCGTTATTCTGGC | |
| | ATACTTGGTCGTCGTCTTAATGATTTAATGACTCATAAAGCTGAACA | |
| | AGAGCGTAAACACTCATCAAGTAGTTTAGAAAGCTATATGAAGGAA | |
| | TATAACGTTAACGAAGAGTATGCTCAAACACTTATTTACAAAGAGGT | |
| | TGAAGACGTTTGGAAGGACATTAACCGTGAATACTTAACAACTAAA | |
| | AACATTCCACGTCCTCTTTTAATGGCTGTAATATACTTATGTCAATTC | |
| | TTAGAAGTACAATACGCTGGAAAAGATAACTTTACACGTATGGGTG | |
| | ATGAATATAAACACTTAATAAAGAGTTTATTAGTTTATCCTATGTCA | |
| | ATAGGTACCGGTGAAAACTTATACTTTCAAGGCTCAGGTGGCGGTG | |
| | GAAGTGATTACAAAGATGATGATGATAAAGGAACCGGTTAA | |
| 84 | ATGGTACCAGCAGGTGTATCAGCTGTGTCAAAAGTTTCTTCATTAGT | Bisabolene |
| | ATGTGACTTAAGTAGTACTAGCGGCTTAATTCGTAGAACTGCAAATC | (A. grandis) |
| | CTCACCCTAATGTATGGGGTTATGACTTAGTTCATTCTTTAAAATCTC | |
| | CATATATTGATAGTAGCTATCGTGAACGTGCTGAAGTGCTTGTAAGT | |
| | GAAATAAAAGCTATGTTAAATCCAGCAATTACTGGAGATGGTGAAT | |
| | CAATGATTACACCTTCAGCTTATGACACTGCTTGGGTTGCACGTGTA | |
| | CCAGCAATTGATGGTAGCGCACGTCCACAATTTCCACAAACAGTAG | |
| | ATTGGATTTTAAAGAATCAATTAAAAGATGGTTCTTGGGGTATTCAA | |
| | TCACACTTTTTACTTTCAGACCGTTTATTAGCTACTCTTAGCTGTGTT | |
| | TTAGTTTTACTTAAATGGAATGTTGGTGATTTACAGGTTGAGCAAGG | |
| | TATTGAGTTTATTAAGTCAAACCTTGAATTAGTAAAAGATGAAACTG | |
| | ATCAAGATTCTTTAGTGACTGATTTTGAGATTATTTTCCCTAGCTTAC | |
| | TTCGTGAGGCCCAAAGTTTACGTTTAGGTCTTCCATACGATTTACCTT | |
| | ACATCCACTTATTACAAACAAAACGTCAGGAACGTTTAGCAAAATT | |
| | AAGCCGTGAAGAAATATATGCAGTTCCAAGTCCACTTTTATATTCTT | |
| | TAGAGGGTATTCAAGATATTGTTGAGTGGGAACGTATTATGGAAGT | |
| | ACAATCTCAGGATGGATCATTTTTAAGTTCTCCAGCATCAACCGCAT | |
| | GTGTTTTTATGCATACAGGTGACGCTAAGTGTTTAGAATTTCTTAAC | |
| | AGTGTAATGATTAAGTTTGGTAATTTTGTACCATGCCTTTATCCTGTA | |
| | GATTTATTAGAACGTTTACTTATAGTAGATAATATAGTTCGTCTTGGT | |
| | ATTTACCGTCACTTCGAAAAAGAAATTAAAGAAGCATTAGATTATGT | |
| | ATATCGCCATTGGAATGAACGTGGTATTGGTTGGGGTCGTTTAAATC | |
| | CAATTGCTGACTTAGAAACAACTGCTTTAGGTTTTCGTTTATTACGTT | |
| | TACACCGTTATAATGTATCTCCAGCAATCTTTGATAATTTCAAAGAT | |
| | GCCAATGGCAAATTCATTTGTAGCACTGGTCAGTTTAATAAGGATGT | |
| | GGCTTCAATGTTAAACTTATACCGTGCATCACAATTAGCATTCCCAG | |
| | GCGAAAACATTTTAGATGAAGCTAAATCTTTTGCCACCAAATACTTA | |
| | CGTGAAGCCCTTGAAAAATCTGAAACTTCATCAGCTTGGAACAATA | |
| | AACAGAATTTAAGTCAAGAAATCAAGTATGCATTAAAAACTTCATG | |
| | GCACGCTTCTGTACCACGTGTTGAAGCAAAACGTTATTGTCAAGTTT | |
| | ATCGTCCTGATTACGCTCGTATTGCTAAGTGTGTATACAAATTACCA | |
| | TACGTTAACAACGAAAAATTCTTAGAATTAGGTAAATTAGATTTTAA | |
| | CATCATTCAATCAATTCATCAAGAAGAAATGAAAAATGTGACAAGT | |
| | TGGTTTCGTGATTCTGGCTTACCATTATTTACTTTCGCTCGCGAACGT | |
| | CCTTTAGAATTTTACTTCTTAGTTGCTGCTGGTACTTATGAACCTCAA | |
| | TATGCTAAATGTCGTTTCTTATTCACAAAAGTAGCTTGTCTTCAAAC | |
| | AGTATTAGACGATATGTACGATACTTACGGTACTTTAGACGAATTAA | |
| | AACTTTTTACCGAGGCTGTGCGTCGTTGGGATTTATCTTTTACAGAA | |
| | AATTTACCTGACTATATGAAATTATGTTATCAAATCTATTATGACAT | |
| | CGTTCATGAAGTGGCTTGGGAAGCTGAAAAAGAACAAGGTAGAGAA | |
| | TTAGTGTCATTCTTCCGTAAAGGCTGGGAAGACTACTTATTAGGTTA | |
| | CTATGAAGAAGCAGAATGGTTAGCAGCAGAATACGTTCCAACATTA | |
| | GATGAATACATTAAAAACGGTATTACATCAATCGGCCAACGTATCTT | |
| | ATTACTTTCAGGTGTGTTAATTATGGATGGCCAACTTTTATCACAAG | |
| | AAGCATTAGAAAAAGTTGATTACCCTGGTCGTCGTGTTTTAACTGAG | |
| | TTAAACTCACTTATTAGCCGTTTAGCTGACGACACTAAAACTTATAA | |
| | AGCAGAAAAAGCTCGTGGAGAATTAGCCTCATCAATTGAATGCTAC | |
| | ATGAAAGATCATCCTGAATGTACAGAAGAAGAAGCCTTAGACCACA | |
| | TTTATTCTATTCTTGAACCAGCCGTAAAAGAATTAACTCGTGAATTT | |
| | CTTAAACCAGACGACGTTCCATTTGCTTGTAAAAAGATGTTATTCGA | |
| | AGAAACTCGTGTTACAATGGTGATCTTTAAAGATGGTGATGGTTTTG | |
| | GTGTATCTAAGTTAGAAGTTAAAGATCACATCAAAGAATGCTTAATT | |
| | GAACCATTACCATTAGGTACCGGTGAAAACTTATACTTTCAAGGCTC | |
| | AGGTGGCGGTGGAAGTGATTACAAAGATGATGATGATAAAGGAACC | |
| | GGTTAA | |
| 85 | ATGGTACCAACTATGATGAATATGAATTTTAAGTACTGTCACAAGAT | Diapophytoene |
| | TATGAAGAAACATTCAAAATCATTCAGTTATGCTTTTGACTTATTAC | (S. aureus) |
| | CAGAAGACCAACGTAAAGCTGTTTGGGCAATTTACGCCGTGTGCCG | |
| | CAAAATTGATGATTCTATTGATGTATATGGTGATATTCAATTCTTAA | |
| | ATCAGATTAAAGAAGACATACAAAGTATTGAAAAATATCCATACGA | |
| | ACATCATCATTTTCAATCTGACAGACGTATTATGATGGCCTTACAGC | |
| | ATGTTGCTCAGCATAAAAACATTGCATTTCAATCATTCTACAATTTA | |
| | ATTGACACAGTATATAAAGATCAACACTTTACAATGTTTGAAACAGA | |
| | TGCTGAACTTTTTGGTTATTGTTACGGTGTAGCTGGTACTGTGGGTG | |
| | AAGTTTTAACTCCTATATTATCTGATCACGAAACACATCAAACTTAT | |
| | GACGTTGCCCGTCGTTTAGGAGAGTCATTACAGTTAATCAATATTCT | |
| | TAGAGATGTAGGTGAAGACTTTGACAACGAACGTATTTACTTCTCTA | |
| | AACAACGTTTAAAACAATACGAAGTAGATATTGCAGAAGTGTACCA | |
| | AAATGGTGTAAACAATCACTATATTGATTTATGGGAATATTACGCTG | |
| | CAATTGCTGAAAAGGATTTTCAAGATGTTATGGACCAAATTAAAGTT | |
| | TTCTCTATTGAAGCTCAGCCAATTATTGAGTTAGCTGCACGTATTTAT | |
| | ATCGAAATTTTAGATGAAGTACGTCAAGCTAACTACACATTACATGA | |
| | ACGTGTTTTTGTAGATAAACGTAAAAAGGCTAAACTTTTTCACGAAA | |
| | ATAAAGGTACCGGTGAAAACTTATACTTTCAAGGCTCAGGTGGCGG | |
| | TGGAAGTGATTACAAAGATGATGATGATAAAGGAACCGGTTAA | |
| 86 | ATGGTACCAAAAATTGCTGTTATTGGTGCTGGTGTTACCGGATTAGC | Diapophytoene |
| | TGCTGCTGCTCGTATTGCTAGCCAAGGTCATGAAGTTACAATCTTCG | desaturase |
| | AAAAAAACAATAATGTAGGTGGTCGTATGAATCAATTAAAAAAAGA | (S. aureus) |
| | TGGTTTTACATTCGATATGGGACCTACAATTGTTATGATGCCAGATG | |
| | TATATAAAGATGTATTTACTGCTTGCGGTAAAAACTATGAAGATTAT | |
| | ATAGAGTTACGTCAACTTCGTTACATTTATGACGTATATTTCGATCA | |
| | CGATGATCGTATTACTGTTCCAACTGATTTAGCTGAATTACAACAAA | |
| | TGTTAGAATCAATTGAACCTGGTAGTACACACGGATTTATGTCATTT | |
| | TTAACAGATGTGTACAAGAAATATGAAATCGCTCGCAGATATTTCTT | |
| | AGAACGTACTTACCGTAAACCATCAGACTTCTACAATATGACCTCTT | |
| | TAGTACAAGGTGCTAAACTTAAAACTTTAAATCACGCTGATCAACTT | |
| | ATCGAACACTACATTGATAACGAAAAGATTCAAAAACTTTTAGCATT | |
| | CCAAACTCTTTATATCGGCATTGATCCAAAGCGTGGTCCTAGTTTAT | |
| | ATAGTATTATTCCTATGATTGAAATGATGTTCGGTGTACATTTTATCA | |
| | AAGGTGGTATGTATGGTATGGCTCAAGGATTAGCTCAACTTAACAA | |
| | AGATTTAGGTGTTAATATTGAATTAAATGCTGAAATTGAACAAATCA | |
| | TTATCGATCCTAAATTCAAACGCGCAGATGCAATTAAAGTTAATGGT | |
| | GACATTCGCAAATTTGATAAGATTTTATGTACTGCTGACTTTCCTTCA | |
| | GTTGCCGAATCACTTATGCCAGATTTCGCACCTATCAAAAAGTACCC | |
| | TCCACATAAAATTGCAGATTTAGATTATTCTTGTTCAGCTTTTCTTAT | |
| | GTATATTGGTATTGACATCGACGTAACTGACCAAGTTCGTTTACATA | |
| | ACGTAATTTTTAGCGACGATTTTCGTGGAAATATTGAAGAAATTTTC | |
| | GAAGGTCGCTTAAGTTACGACCCATCAATCTATGTTTATGTACCAGC | |
| | TGTAGCCGATAAATCTTTAGCTCCTGAAGGTAAAACAGGCATTTATG | |
| | TGTTAATGCCTACTCCTGAACTTAAAACAGGATCAGGTATTGACTGG | |
| | TCAGATGAGGCTTTAACTCAACAAATTAAAGAAATCATTTATCGTAA | |
| | ATTAGCAACAATTGAAGTATTTGAAGACATTAAATCACACATTGTAT | |
| | CAGAAACAATTTTTACTCCTAATGACTTTGAACAAACCTATCACGCT | |
| | AAATTTGGTTCTGCTTTCGGTTTAATGCCCACCTTAGCACAATCTAAT | |
| | TATTACAGACCTCAAAATGTGTCACGTGATTATAAAGACTTATATTT | |
| | CGCAGGTGCATCAACACATCCAGGTGCTGGAGTTCCAATTGTATTAA | |
| | CAAGTGCCAAGATAACAGTAGACGAAATGATTAAAGATATTGAGCG | |
| | TGGTGTGGGTACCGGTGAAAACTTATACTTTCAAGGCTCAGGTGGCG | |
| | GTGGAAGTGATTACAAAGATGATGATGATAAAGGAACCGGTTAA | |
| 87 | ATGGTACCAGCATTTGACTTCGATGGTTACATGCTTCGTAAAGCTAA | GPPS-LSU |
| | ATCTGTAAATAAAGCTCTTGAAGCTGCAGTACAAATGAAAGAACCA | (M. spicata) |
| | TTAAAAATTCATGAAAGTATGCGTTATTCTTTATTAGCTGGTGGTAA | |
| | ACGTGTACGTCCAATGTTATGTATTGCAGCTTGTGAATTAGTTGGTG | |
| | GTGACGAAAGTACTGCTATGCCTGCTGCTTGCGCTGTAGAAATGATT | |
| | CATACTATGAGTTTAATGCATGATGATTTACCATGTATGGATAATGA | |
| | CGATTTACGTCGTGGTAAACCAACAAACCACATGGCATTTGGTGAA | |
| | AGTGTAGCAGTATTAGCAGGTGATGCATTATTATCTTTTGCTTTTGA | |
| | ACATGTAGCAGCAGCAACAAAAGGTGCTCCTCCAGAACGTATTGTT | |
| | AGAGTTTTAGGTGAACTTGCAGTTTCTATTGGTTCAGAAGGTTTAGT | |
| | TGCTGGACAAGTAGTTGACGTTTGTTCTGAAGGTATGGCTGAGGTTG | |
| | GTTTAGATCATTTAGAATTTATTCATCACCACAAAACTGCTGCTTTAT | |
| | TACAAGGTTCTGTAGTATTAGGTGCAATATTAGGTGGTGGAAAAGA | |
| | AGAAGAGGTAGCAAAACTTCGTAAATTCGCTAACTGCATTGGTTTAC | |
| | TTTTCCAAGTAGTAGATGATATTCTTGATGTAACAAAATCATCTAAA | |
| | GAATTAGGTAAAACAGCAGGTAAAGATTTAGTTGCTGATAAAACTA | |
| | CTTATCCAAAATTAATCGGTGTTGAGAAAAGTAAAGAGTTCGCAGA | |
| | CCGTTTAAATCGTGAAGCTCAAGAACAACTTCTTCATTTTCATCCAC | |
| | ATAGAGCAGCACCTTTAATCGCTTTAGCAAACTATATTGCTTATCGT | |
| | GATAATGGTACCGGTGAAAATTTATATTTTCAAGGTTCAGGTGGCGG | |
| | AGGTTCTGATTATAAAGATGATGATGATAAAGGAACCGGTTAA | |
| 88 | ATGGTACCAAGTCAACCTTACTGGGCAGCAATTGAGGCAGATATTG | GPPS-SSU |
| | AACGTTACTTAAAAAAATCAATTACAATTCGTCCACCAGAAACTGTA | (M. spicata) |
| | TTTGGTCCAATGCACCACTTAACTTTTGCTGCACCAGCTACAGCTGC | |
| | TAGTACTTTATGTTTAGCAGCATGTGAACTTGTAGGTGGTGATCGTA | |
| | GTCAAGCTATGGCTGCAGCAGCAGCAATCCATCTTGTTCATGCAGCT | |
| | GCTTATGTACATGAACATTTACCATTAACTGATGGTAGTCGTCCAGT | |
| | AAGTAAACCAGCTATCCAACATAAATATGGTCCAAATGTAGAATTA | |
| | CTTACAGGTGACGGTATTGTACCATTTGGTTTTGAATTATTAGCAGG | |
| | TTCTGTTGATCCAGCACGTACAGATGATCCAGACCGTATTTTACGTG | |
| | TAATAATTGAAATAAGTCGTGCTGGTGGTCCAGAAGGTATGATTAGT | |
| | GGTTTACATCGTGAAGAAGAGATTGTAGATGGTAATACTTCTCTTGA | |
| | TTTTATTGAATACGTTTGCAAAAAAAAATATGGTGAAATGCACGCAT | |
| | GTGGTGCTGCATGCGGTGCAATTTTAGGTGGTGCAGCTGAAGAAGA | |
| | AATTCAAAAACTTCGTAACTTCGGATTATATCAAGGAACTTTACGTG | |
| | GTATGATGGAGATGAAAAACTCACACCAACTTATTGACGAAAATAT | |
| | CATTGGCAAACTTAAAGAATTAGCTTTAGAAGAATTAGGTGGATTTC | |
| | ATGGTAAAAATGCTGAATTAATGTCTAGTTTAGTAGCAGAACCATCA | |
| | TTATATGCTGCTGGTACCGGTGAAAATTTATACTTTCAAGGTTCTGG | |
| | TGGTGGTGGCAGTGATTATAAAGACGATGATGACAAAGGAACCGGT | |
| | TAA | |
| 89 | ATGGTACCACTTTTATCTAACAAATTAAGAGAGATGGTTTTAGCAGA | GPPS (A. thalania) |
| | AGTTCCTAAATTAGCATCTGCTGCTGAATATTTCTTTAAACGTGGTGT | |
| | TCAGGGTAAACAATTCCGTTCAACAATTTTATTATTAATGGCAACAG | |
| | CTCTTGACGTTCGTGTTCCAGAAGCATTAATTGGTGAATCTACTGAT | |
| | ATTGTAACATCTGAATTACGTGTACGTCAACGTGGCATTGCTGAAAT | |
| | TACAGAAATGATTCATGTAGCATCACTTCTTCACGATGACGTTCTTG | |
| | ACGATGCTGATACTCGTCGTGGTGTTGGTAGTCTTAATGTTGTAATG | |
| | GGAAACAAAATGTCAGTTTTAGCAGGTGACTTCTTACTTTCTCGTGC | |
| | TTGTGGTGCTCTTGCAGCTCTTAAAAACACAGAAGTTGTAGCATTAT | |
| | TAGCTACAGCAGTAGAACACTTAGTTACTGGTGAGACAATGGAAAT | |
| | AACTTCATCAACTGAACAACGTTATTCTATGGATTACTACATGCAGA | |
| | AAACTTATTACAAAACTGCTTCATTAATTTCAAATTCATGTAAAGCA | |
| | GTTGCTGTATTAACAGGTCAAACAGCTGAAGTTGCAGTATTAGCTTT | |
| | TGAATATGGTCGTAATTTAGGTTTAGCTTTCCAGTTAATTGACGACA | |
| | TTTTAGATTTCACAGGCACATCTGCTAGTTTAGGAAAAGGTTCTTTA | |
| | TCAGATATACGTCATGGTGTTATTACTGCTCCTATCTTATTTGCAATG | |
| | GAAGAATTTCCTCAATTAAGAGAAGTAGTAGATCAAGTAGAAAAAG | |
| | ATCCAAGAAATGTAGACATAGCTTTAGAATATTTAGGTAAAAGTAA | |
| | AGGTATTCAACGTGCTCGTGAATTAGCAATGGAACACGCAAATTTA | |
| | GCTGCTGCAGCTATTGGTTCTTTACCTGAAACAGATAACGAAGATGT | |
| | TAAACGTTCACGTCGTGCTTTAATTGATTTAACACACAGAGTAATTA | |
| | CACGTAACAAAGGTACCGGTGAGAATTTATACTTTCAAGGTAGTGGT | |
| | GGAGGAGGTAGTGACTATAAAGATGATGACGATAAAGGAACCGGTT | |
| | AA | |
| 90 | ATGGTACCAGTAGTTTCTGAACGTTTAAGACATTCTGTAACAACTGG | GPPS (C. reinhardtii) |
| | TATTCCAGCATTAAAAACAGCAGCTGAATATTTCTTTCGTCGTGGTA | |
| | TCGAAGGAAAACGTTTAAGACCTACATTAGCATTATTAATGAGTAGT | |
| | GCTTTATCACCAGCTGCTCCATCACCAGAGTATTTACAAGTTGATAC | |
| | AAGACCTGCTGCAGAACACCCTCATGAAATGCGTCGTCGTCAACAA | |
| | CGTTTAGCTGAAATTGCAGAATTAATCCATGTAGCTTCATTACTTCA | |
| | CGATGATGTTATTGATGACGCACAAACACGTCGTGGTGTTTTAAGTT | |
| | TAAATACATCTGTTGGTAATAAAACAGCTATCTTAGCAGGTGATTTC | |
| | TTATTAGCTCGTGCATCTGTAACATTAGCTAGTTTAAGAAACTCTGA | |
| | AATTGTAGAATTAATGTCACAGGTTTTAGAACACTTAGTATCTGGTG | |
| | AAATTATGCAAATGACTGCTACTTCAGAACAACTTTTAGATTTAGAA | |
| | CATTATTTAGCAAAAACATATTGTAAAACTGCTTCATTAATGGCTAA | |
| | TAGTTCTCGTTCTGTTGCAGTTCTTGCAGGTGCAGCTCCTGAAGTTTG | |
| | TGATATGGCATGGTCATACGGTCGTCATTTAGGTATTGCTTTCCAAG | |
| | TAGTTGACGATTTATTAGATTTAACAGGTTCATCTTCTGTTTTAGGTA | |
| | AACCTGCTTTAAACGATATGCGTTCTGGTTTAGCAACAGCACCAGTA | |
| | TTATTCGCTGCACAAGAAGAACCTGCATTACAGGCTCTTATATTACG | |
| | TCGTTTTAAACACGACGGTGACGTAACAAAAGCAATGTCATTAATTG | |
| | AACGTACACAAGGCTTACGTCGTGCTGAAGAACTTGCAGCACAACA | |
| | CGCAAAAGCTGCTGCTGATATGATTCGTTGCTTACCTACAGCTCAAT | |
| | CAGACCATGCAGAAATTGCTCGTGAAGCATTAATTCAAATTACACAT | |
| | CGTGTTTTAACACGTAAAAAAGGTACCGGTGAAAACTTATACTTTCA | |
| | AGGTTCTGGTGGTGGTGGATCAGATTATAAAGATGATGATGACAAA | |
| | GGAACCGGTTAA | |
| 91 | ATGGTACCAGATTTTCCACAACAATTAGAAGCATGTGTTAAACAAGC | FPP (E. coli) |
| | AAATCAAGCATTATCACGTTTCATCGCACCACTTCCATTCCAAAATA | |
| | CTCCTGTTGTTGAAACAATGCAATATGGTGCATTATTAGGAGGTAAA | |
| | AGATTAAGACCATTTCTTGTATATGCAACAGGTCACATGTTTGGAGT | |
| | ATCTACTAACACATTAGATGCTCCAGCTGCTGCAGTTGAATGTATTC | |
| | ATGCATATAGTTTAATTCATGATGATTTACCTGCAATGGATGATGAT | |
| | GACTTAAGAAGAGGTTTACCTACATGTCATGTTAAATTTGGTGAAGC | |
| | TAATGCTATTTTAGCTGGCGATGCACTTCAAACTCTTGCATTCAGTAT | |
| | TTTATCAGATGCTGATATGCCAGAAGTTTCAGATCGTGATCGTATTT | |
| | CTATGATATCTGAATTAGCTTCTGCTAGTGGTATTGCTGGTATGTGC | |
| | GGTGGCCAAGCTCTTGATTTAGACGCAGAAGGAAAACACGTTCCTTT | |
| | AGATGCTTTAGAGCGTATACATCGTCACAAAACAGGAGCTTTAATTA | |
| | GAGCTGCTGTTCGTCTTGGTGCTTTATCAGCTGGAGACAAAGGTCGT | |
| | CGTGCTTTACCAGTTTTAGACAAATACGCTGAAAGTATTGGTTTAGC | |
| | TTTTCAAGTTCAGGATGATATCTTAGATGTTGTAGGTGATACTGCTA | |
| | CTTTAGGTAAACGTCAAGGTGCTGATCAACAGTTAGGCAAATCTACA | |
| | TACCCAGCACTTTTAGGTTTAGAACAAGCTCGTAAAAAAGCAAGAG | |
| | ACTTAATTGACGATGCTCGTCAAAGTCTTAAACAATTAGCAGAACAA | |
| | TCACTTGATACAAGTGCTTTAGAAGCATTAGCAGATTACATTATTCA | |
| | ACGTAATAAAGGTACCGGTGAAAATTTATATTTTCAAGGTTCTGGTG | |
| | GTGGAGGTTCAGACTATAAAGATGACGATGATAAAGGAACCGGTTAA | |
| 92 | ATGGTACCAAGTGTTAGTTGTTGTTGTAGAAATTTAGGAAAAACTAT | FPP (A. thalania) |
| | CAAAAAAGCTATTCCAAGTCACCACTTACATTTACGTTCTTTAGGTG | |
| | GTAGTTTATATAGAAGACGTATTCAATCATCTTCAATGGAAACAGAC | |
| | TTAAAATCTACATTCTTAAATGTTTATTCAGTTCTTAAATCAGATTTA | |
| | TTACACGACCCATCATTTGAATTTACAAATGAAAGTCGTTTATGGGT | |
| | AGATAGAATGCTTGATTATAATGTTCGTGGCGGTAAACTTAATCGTG | |
| | GTCTTTCTGTAGTAGACTCTTTCAAATTACTTAAACAAGGTAATGAT | |
| | TTAACTGAACAAGAAGTTTTCTTATCTTGTGCATTAGGTTGGTGTATT | |
| | GAGTGGTTACAGGCTTACTTTTTAGTTCTTGATGATATTATGGATAAT | |
| | TCAGTTACACGTCGTGGTCAACCTTGTTGGTTTCGTGTACCACAAGT | |
| | TGGTATGGTAGCTATTAATGATGGCATTCTTCTTCGTAACCATATTCA | |
| | TCGTATTCTTAAAAAACACTTCCGTGATAAACCATATTATGTAGATT | |
| | TAGTTGACCTTTTCAATGAAGTAGAGTTACAAACTGCATGTGGACAA | |
| | ATGATTGATTTAATCACAACATTTGAAGGTGAAAAAGACTTAGCTAA | |
| | ATATAGTTTATCAATTCACCGTCGTATTGTTCAATACAAAACTGCAT | |
| | ATTACTCATTCTATTTACCAGTTGCATGTGCTCTTTTAATGGCTGGCG | |
| | AAAATTTAGAAAACCACATTGATGTTAAAAATGTATTAGTAGATATG | |
| | GGTATTTACTTTCAAGTTCAGGATGATTATTTAGACTGTTTTGCTGAT | |
| | CCTGAAACATTAGGTAAAATTGGCACTGATATTGAGGACTTTAAATG | |
| | TTCTTGGTTAGTTGTAAAAGCATTAGAACGTTGTAGTGAAGAACAAA | |
| | CAAAAATTCTTTACGAAAACTATGGCAAACCTGATCCATCTAATGTT | |
| | GCTAAAGTAAAAGATTTATACAAAGAATTAGATTTAGAAGGCGTTTT | |
| | CATGGAATATGAATCTAAATCATACGAGAAATTAACTGGTGCTATCG | |
| | AAGGTCACCAATCTAAAGCAATTCAAGCTGTTCTTAAATCTTTCTTA | |
| | GCAAAAATCTATAAACGTCAAAAAGGTACCGGTGAAAACTTATACT | |
| | TTCAAGGTAGTGGTGGCGGTGGTAGTGATTATAAAGATGATGATGA | |
| | TAAAGGAACCGGTTAA | |
| 93 | ATGGTACCAGCTGATCTTAAATCAACATTCTTAGATGTTTATTCAGT | FPP (A. thalania) |
| | ATTAAAAAGTGATTTATTACAAGATCCATCTTTTGAATTTACACACG | |
| | AAAGTCGTCAATGGTTAGAACGTATGTTAGATTATAATGTTCGTGGA | |
| | GGCAAATTAAACAGAGGTTTAAGTGTAGTAGACAGTTACAAACTTTT | |
| | AAAACAAGGTCAAGACTTAACAGAAAAAGAAACATTTTTATCTTGT | |
| | GCTTTAGGTTGGTGTATTGAATGGTTACAAGCATACTTCTTAGTTTTA | |
| | GACGATATTATGGATAATTCTGTAACTAGACGTGGTCAACCATGTTG | |
| | GTTTCGTAAACCAAAAGTAGGTATGATTGCTATTAATGATGGAATAC | |
| | TTCTTCGTAACCACATTCATCGTATTCTTAAAAAACACTTTCGTGAA | |
| | ATGCCTTATTATGTAGACCTTGTAGACTTATTTAACGAAGTAGAATT | |
| | TCAAACAGCTTGTGGTCAAATGATTGACTTAATTACAACATTTGATG | |
| | GTGAAAAAGACCTTTCAAAATATTCACTTCAGATTCACCGTCGTATT | |
| | GTTGAGTACAAAACAGCATACTACTCTTTCTATTTACCTGTAGCATG | |
| | TGCTTTACTTATGGCAGGTGAAAATTTAGAAAATCACACAGATGTTA | |
| | AAACTGTATTAGTTGATATGGGTATCTATTTCCAAGTTCAAGATGAT | |
| | TATTTAGATTGCTTCGCTGATCCAGAAACATTAGGTAAAATTGGTAC | |
| | AGATATTGAAGACTTTAAATGTAGTTGGTTAGTAGTAAAAGCATTAG | |
| | AACGTTGTAGTGAAGAACAAACAAAAATTCTTTACGAAAATTATGG | |
| | AAAAGCTGAACCTTCAAATGTAGCTAAAGTTAAAGCATTATACAAA | |
| | GAATTAGATTTAGAGGGTGCATTTATGGAATATGAAAAAGAATCAT | |
| | ACGAGAAACTTACAAAACTTATTGAAGCACATCAATCAAAAGCTAT | |
| | TCAAGCAGTTCTTAAATCTTTCTTAGCTAAAATTTATAAACGTCAAA | |
| | AAGGTACCGGTGAAAACTTATACTTTCAAGGCTCTGGAGGTGGTGGT | |
| | TCAGACTATAAAGATGATGATGATAAAGGAACCGGTTAA | |
| 94 | ATGGTACCAAGTGGCGAACCTACTCCAAAAAAAATGAAAGCAACAT | FPP (C. reinhardtii) |
| | ACGTTCACGACCGTGAAAACTTTACAAAAGTATACGAAACTCTTCGT | |
| | GACGAATTACTTAACGATGATTGTCTTAGTCCAGCTGGTTCACCTCA | |
| | GGCTCAAGCTGCTCAAGAGTGGTTTAAAGAAGTTAATGATTATAATG | |
| | TTCCTGGTGGAAAACTTAACCGTGGTATGGCTGTATATGACGTTTTA | |
| | GCTTCAGTTAAAGGTCCAGATGGTTTAAGTGAAGACGAAGTATTTAA | |
| | AGCTAACGCTCTTGGTTGGTGTATTGAGTGGTTACAAGCATTTTTCTT | |
| | AGTTGCTGATGATATAATGGATGGTTCAATTACACGTCGTGGCCAAC | |
| | CTTGTTGGTACAAACAACCTAAAGTTGGTATGATTGCTTGTAATGAT | |
| | TACATCTTATTAGAATGCTGTATTTACTCAATTCTTAAAAGACATTTT | |
| | AGAGGTCACGCTGCATACGCTCAACTTATGGACCTTTTCCATGAAAC | |
| | TACATTCCAGACTTCACACGGTCAATTATTAGATTTAACAACAGCAC | |
| | CTATCGGTTCTGTAGACTTATCAAAATATACAGAAGATAATTACCTT | |
| | CGTATTGTAACATATAAAACTGCATACTATTCTTTTTATTTACCTGTA | |
| | GCATGTGGTATGGTATTAGCTGGCATTACAGATCCAGCTGCTTTTGA | |
| | TCTTGCAAAAAATATTTGTGTTGAAATGGGTCAATATTTCCAGATTC | |
| | AAGACGATTATTTAGATTGCTATGGTGACCCTGAGGTTATTGGTAAA | |
| | ATCGGTACAGACATAGAAGACAACAAATGTAGTTGGTTAGTTTGCA | |
| | CAGCTCTTAAAATCGCAACAGAAGAACAAAAAGAGGTTATAAAAGC | |
| | TAATTATGGTCACAAAGAGGCTGAATCAGTAGCAGCAATTAAAGCA | |
| | TTATACGTTGAATTAGGTATTGAACAACGTTTTAAAGACTATGAAGC | |
| | TGCATCATACGCAAAATTAGAAGGTACAATTAGTGAACAAACTTTAT | |
| | TACCTAAAGCAGTATTTACTTCTTTATTAGCTAAAATCTATAAAAGA | |
| | AAAAAAGGTACCGGTGAGAACTTATACTTTCAAGGTAGTGGAGGTG | |
| | GTGGTTCAGACTATAAAGATGATGATGATAAAGGAACCGGTTAA | |
| 95 | ATGGTACCACAAACTGAACATGTTATCTTATTAAACGCTCAAGGTGT | IPP |
| | TCCTACAGGTACATTAGAAAAATATGCTGCACACACTGCTGATACTC | isomerase |
| | GTTTACACTTAGCTTTCTCATCTTGGTTATTCAATGCTAAAGGTCAAC | (E. coli) |
| | TTTTAGTTACAAGACGTGCATTAAGTAAAAAAGCATGGCCTGGTGTT | |
| | TGGACTAACTCAGTTTGTGGTCATCCACAATTAGGTGAAAGTAATGA | |
| | AGATGCAGTTATACGTCGTTGCAGATATGAATTAGGTGTTGAAATAA | |
| | CTCCACCAGAATCAATTTATCCAGATTTCCGTTATCGTGCAACTGAT | |
| | CCTAGTGGTATCGTTGAAAACGAAGTATGTCCTGTTTTTGCTGCACG | |
| | TACAACAAGTGCATTACAAATTAATGATGATGAAGTAATGGATTATC | |
| | AATGGTGTGACTTAGCTGATGTTTTACATGGTATTGATGCAACACCA | |
| | TGGGCATTTTCACCATGGATGGTAATGCAAGCAACAAATCGTGAAG | |
| | CACGTAAAAGATTAAGTGCTTTTACACAGTTAAAAGGTACCGGTGA | |
| | AAACTTATACTTTCAAGGTAGTGGAGGTGGTGGTTCTGACTATAAAG | |
| | ATGACGATGATAAAGGAACCGGTTAA | |
| 96 | ATGGTACCACTTCGTAGTTTATTAAGAGGTTTAACACACATTCCTCG | IPP |
| | TGTTAATAGTGCTCAGCAACCTTCTTGCGCTCACGCTCGTCTTCAATT | isomerase |
| | TAAACTTCGTTCTATGCAATTATTAGCAGAAAACCGTACAGATCACA | (H. pluvalis) |
| | TGCGTGGTGCTTCTACATGGGCAGGTGGTCAGTCTCAAGATGAATTA | |
| | ATGCTTAAAGATGAATGTATCTTAGTAGATGCTGATGATAACATTAC | |
| | TGGTCACGCTTCTAAATTAGAATGTCACAAATTTCTTCCACATCAAC | |
| | CAGCTGGATTATTACACCGTGCTTTTTCTGTATTTCTTTTCGACGATC | |
| | AAGGTCGTTTACTTTTACAACAACGTGCTCGTAGTAAAATTACATTT | |
| | CCATCTGTATGGGCTAATACATGTTGTAGTCATCCATTACATGGTCA | |
| | AACACCAGATGAAGTAGATCAACAATCACAAGTAGCAGACGGAACT | |
| | GTACCAGGTGCAAAAGCTGCTGCAATCAGAAAATTAGAACATGAAT | |
| | TAGGTATTCCAGCTCACCAATTACCAGCATCAGCTTTTCGTTTCTTAA | |
| | CACGTCTTCACTATTGTGCAGCTGACGTTCAACCTGCAGCAACACAA | |
| | TCTGCATTATGGGGTGAACACGAAATGGATTACATTTTATTCATTAG | |
| | AGCTAATGTTACACTTGCTCCTAATCCTGACGAAGTAGATGAGGTAC | |
| | GTTATGTAACTCAAGAAGAATTAAGACAAATGATGCAACCAGATAA | |
| | TGGTTTACAATGGTCACCATGGTTCCGTATTATTGCAGCAAGATTTTT | |
| | AGAACGTTGGTGGGCTGATTTAGATGCTGCATTAAATACAGATAAA | |
| | CATGAAGACTGGGGAACAGTTCATCACATTAACGAAGCTGGTACCG | |
| | GTGAAAACTTATACTTTCAAGGATCAGGAGGCGGTGGAAGTGATTA | |
| | TAAAGATGATGATGATAAAGGAACCGGTTAA | |
| 97 | ATGGTACCAAGAAGATCAGGCAATTATAACCCAACAGCATGGGACT | Limonene |
| | TCAATTATATCCAATCATTAGACAATCAATACAAAAAAGAACGTTAC | (L. angustifolia) |
| | TCTACTCGTCACGCTGAATTAACAGTTCAAGTTAAAAAATTATTAGA | |
| | AGAAGAAATGGAAGCTGTTCAAAAACTTGAACTTATAGAGGATCTT | |
| | AAAAACTTAGGCATTTCTTACCCATTTAAAGATAATATTCAACAAAT | |
| | CTTAAATCAAATTTACAATGAACACAAATGTTGTCACAACTCAGAAG | |
| | TTGAAGAAAAAGACCTTTATTTCACTGCTTTACGTTTTAGATTATTAC | |
| | GTCAACAAGGTTTTGAAGTAAGTCAAGAAGTATTTGATCACTTTAAA | |
| | AACGAAAAAGGTACAGATTTTAAACCTAATTTAGCAGATGATACTA | |
| | AAGGATTATTACAATTATATGAAGCATCATTCTTATTACGTGAAGCA | |
| | GAAGACACATTAGAACTTGCTCGTCAATTCTCTACTAAACTTTTACA | |
| | AAAAAAAGTTGATGAAAACGGTGACGATAAAATTGAAGATAACTTA | |
| | TTACTTTGGATTAGACGTAGTTTAGAATTACCATTACATTGGCGTGT | |
| | ACAAAGATTAGAAGCTCGTGGCTTTTTAGATGCTTACGTTCGTAGAC | |
| | CTGATATGAATCCTATTGTATTTGAATTAGCAAAATTAGACTTTAAC | |
| | ATTACTCAAGCAACACAACAAGAAGAACTTAAAGATTTATCAAGAT | |
| | GGTGGAATAGTACTGGCTTAGCTGAAAAACTTCCTTTTGCTCGTGAT | |
| | CGTGTAGTTGAATCATATTTCTGGGCTATGGGTACTTTTGAACCACA | |
| | TCAATACGGATACCAACGTGAATTAGTTGCTAAAATCATTGCACTTG | |
| | CTACAGTTGTAGACGATGTTTACGATGTATATGGTACTTTAGAGGAA | |
| | TTAGAACTTTTTACTGATGCTATTCGTCGTTGGGACCGTGAATCTATT | |
| | GACCAATTACCATATTACATGCAATTATGTTTTCTTACTGTAAACAA | |
| | CTTTGTTTTTGAGTTAGCTCACGACGTATTAAAAGATAAATCATTCA | |
| | ATTGTTTACCTCATTTACAAAGATCATGGTTAGATTTAGCTGAAGCA | |
| | TACCTTGTAGAAGCAAAATGGTATCATAGTCGTTATACACCTTCTTT | |
| | AGAAGAATATCTTAATATTGCTCGTGTTTCAGTAACATGTCCAACTA | |
| | TTGTTTCTCAAATGTATTTTGCATTACCAATTCCAATCGAAAAACCTG | |
| | TAATTGAGATCATGTACAAATATCACGATATCTTATACTTATCAGGT | |
| | ATGTTATTACGTTTACCAGATGACTTAGGAACAGCATCATTCGAACT | |
| | TAAACGTGGTGATGTACAAAAAGCAGTTCAATGTTATATGAAAGAA | |
| | CGTAATGTTCCTGAAAATGAAGCTCGTGAACATGTTAAATTCTTAAT | |
| | TCGTGAGGCTTCTAAACAAATTAATACAGCAATGGCAACAGACTGT | |
| | CCATTTACAGAAGATTTTGCAGTTGCAGCAGCAAACTTAGGTCGTGT | |
| | AGCAAATTTTGTATATGTTGATGGTGATGGTTTTGGAGTACAACACA | |
| | GTAAAATCTATGAGCAAATTGGTACACTTATGTTTGAACCATATCCA | |
| | GGTACCGGTGAAAACTTATACTTTCAAGGTAGTGGTGGTGGAGGTTC | |
| | TGATTACAAAGACGATGATGATAAAGGAACCGGTTAA | |
| 98 | ATGGTACCAAGAAGAAGTGGAAACTATAAACCTACAATGTGGGATT | Monoterpene |
| | TTCAATTTATTCAAAGTGTAAATAATCTTTACGCTGGTGATAAATAC | (S. lycopersicum) |
| | ATGGAACGTTTCGATGAAGTAAAAAAAGAAATGAAAAAAAACTTAA | |
| | TGATGATGGTTGAGGGTTTAATAGAGGAATTAGATGTTAAATTAGA | |
| | ATTAATAGATAATTTAGAAAGATTAGGTGTTAGTTATCATTTCAAAA | |
| | ATGAAATAATGCAAATCCTTAAATCTGTACACCAGCAAATCACTTGT | |
| | CGTGATAATTCATTATACTCTACTGCATTAAAATTTCGTTTATTACGT | |
| | CAACACGGATTCCACATTAGTCAAGACATCTTTAACGATTTTAAAGA | |
| | TATGAATGGCAATGTTAAACAAAGTATCTGTAACGATACTAAAGGTT | |
| | TATTAGAACTTTATGAAGCATCTTTCTTATCTACTGAATGTGAAACA | |
| | ACACTTAAAAACTTCACTGAAGCACACTTAAAAAATTATGTTTATAT | |
| | TAACCACTCATGTGGAGATCAATACAATAACATAATGATGGAATTA | |
| | GTTGTTCACGCTTTAGAATTACCACGTCACTGGATGATGCCTCGTTT | |
| | AGAGACACGTTGGTATATATCAATTTATGAACGTATGCCTAATGCTA | |
| | ATCCACTTTTACTTGAACTTGCTAAATTAGACTTCAATATTGTTCAAG | |
| | CTACACACCAACAAGACTTAAAATCATTATCACGTTGGTGGAAAAA | |
| | CATGTGTTTAGCTGAAAAATTATCATTTTCTCGTAACCGTTTAGTAG | |
| | AAAATCTTTTCTGGGCAGTTGGAACTAATTTTGAACCACAACACAGT | |
| | TATTTCCGTCGTTTAATCACTAAAATCATTGTTTTTGTTGGTATTATT | |
| | GATGATATTTATGATGTTTACGGCAAACTTGATGAGTTAGAATTATT | |
| | CACTTTAGCTGTACAACGTTGGGATACAAAAGCAATGGAAGACTTA | |
| | CCATATTACATGCAAGTTTGTTATTTAGCTTTAATTAATACAACAAA | |
| | TGATGTTGCTTATGAAGTTCTTCGTAAACATAACATTAATGTATTAC | |
| | CATACTTAACTAAATCTTGGACAGACTTATGTAAATCATATTTACAA | |
| | GAAGCTCGTTGGTACTACAATGGTTACAAACCTTCATTAGAGGAATA | |
| | TATGGATAATGGTTGGATTAGTATAGCAGTTCCTATGGTATTAGCAC | |
| | ATGCACTTTTCTTAGTTACAGATCCAATTACAAAAGAAGCATTAGAA | |
| | TCATTAACAAACTATCCTGATATTATTCGTTGCTCAGCTACAATATTC | |
| | CGTTTAAATGATGATCTTGGTACAAGTTCAGATGAATTAAAACGTGG | |
| | AGATGTACCAAAATCAATTCAATGCTATATGAACGAAAAAGGCGTT | |
| | TCAGAGGAAGAAGCTCGTGAACATATTCGTTTCTTAATCAAAGAAA | |
| | CATGGAAATTCATGAACACTGCACACCATAAAGAGAAAAGTTTATT | |
| | TTGTGAGACATTTGTAGAAATTGCAAAAAATATTGCAACAACAGCTC | |
| | ATTGTATGTACTTAAAAGGTGATTCTCACGGTATTCAAAACACTGAT | |
| | GTTAAAAACTCAATAAGTAATATACTTTTCCATCCAATTATTATCGG | |
| | TACCGGTGAAAACCTTTACTTTCAAGGTTCAGGTGGTGGCGGTTCAG | |
| | ACTATAAAGATGACGATGATAAAGGAACCGGTTAA | |
| 99 | ATGGTACCAAGACGTAGTGGAAATTATGAGCCATCTGCATGGGACT | Terpinolene |
| | TCAATTACTTACAATCTCTTAATAATTATCACCATAAAGAAGAACGT | (O. basilicum) |
| | TACTTACGTCGTCAAGCTGATTTAATTGAAAAAGTAAAAATGATTCT | |
| | TAAAGAAGAGAAAATGGAAGCATTACAGCAATTAGAACTTATAGAC | |
| | GATCTTCGTAATTTAGGTCTTTCATATTGTTTTGATGATCAAATTAAT | |
| | CATATTCTTACAACAATTTACAACCAACATTCTTGTTTCCATTATCAC | |
| | GAAGCTGCAACAAGTGAAGAAGCTAACTTATATTTCACAGCTTTAG | |
| | GTTTCCGTTTACTTCGTGAACACGGATTCAAAGTATCACAAGAAGTA | |
| | TTTGACCGTTTCAAAAATGAAAAAGGTACAGATTTTCGTCCAGATTT | |
| | AGTAGATGATACTCAAGGTTTATTACAACTTTATGAAGCATCTTTCC | |
| | TTCTTCGTGAAGGTGAAGACACTTTAGAATTTGCACGTCAATTTGCT | |
| | ACTAAATTTCTTCAAAAAAAAGTTGAGGAGAAAATGATAGAAGAGG | |
| | AAAATCTTTTATCTTGGACTTTACATTCACTTGAATTACCATTACATT | |
| | GGCGTATACAACGTTTAGAAGCTAAATGGTTTTTAGATGCTTATGCT | |
| | AGTCGTCCTGATATGAATCCAATAATCTTTGAATTAGCAAAATTAGA | |
| | ATTTAACATTGCTCAGGCACTTCAACAAGAAGAACTTAAAGATTTAT | |
| | CAAGATGGTGGAACGATACTGGTATTGCTGAAAAATTACCTTTCGCT | |
| | CGTGATAGAATCGTTGAATCTCATTATTGGGCAATTGGTACTTTAGA | |
| | ACCTTATCAATACCGTTATCAGCGTTCATTAATTGCAAAAATCATTG | |
| | CTTTAACTACAGTTGTTGATGATGTATATGATGTTTACGGTACATTA | |
| | GACGAATTACAGTTATTTACTGATGCAATTCGTCGTTGGGACATTGA | |
| | AAGTATAAATCAATTACCTTCTTATATGCAATTATGTTATTTAGCTAT | |
| | TTATAATTTCGTATCAGAATTAGCTTATGATATTTTCAGAGATAAAG | |
| | GTTTTAATTCTTTACCATATTTACACAAAAGTTGGCTTGACTTAGTTG | |
| | AGGCTTACTTTCAAGAAGCAAAATGGTATCATTCTGGCTACACACCA | |
| | TCATTAGAACAATACTTAAATATCGCTCAAATTTCTGTAGCAAGTCC | |
| | AGCTATATTAAGTCAAATTTACTTTACTATGGCTGGTTCAATTGATA | |
| | AACCAGTAATCGAATCAATGTACAAATATAGACACATTTTAAACTTA | |
| | TCTGGTATATTACTTAGATTACCAGATGACTTAGGTACTGCTAGTGA | |
| | TGAATTAGGTCGTGGTGATTTAGCAAAAGCAATGCAATGTTACATGA | |
| | AAGAGCGTAACGTTTCTGAAGAAGAAGCTCGTGATCATGTACGTTTC | |
| | TTAAATCGTGAGGTTTCAAAACAAATGAATCCTGCTCGTGCTGCTGA | |
| | TGATTGTCCATTCACTGATGATTTTGTAGTAGCTGCTGCTAATTTAGG | |
| | AAGAGTTGCAGATTTCATGTATGTTGAAGGCGATGGTTTAGGTTTAC | |
| | AATACCCAGCTATCCACCAACACATGGCAGAACTTTTATTTCACCCT | |
| | TACGCAGGTACCGGTGAAAACTTATACTTTCAAGGTTCAGGTGGTGG | |
| | AGGTTCTGACTATAAAGATGATGATGATAAAGGAACCGGTTAA | |
| 100 | ATGGTACCAAGAAGATCAGGAAATTATCAACCTAGTGCATGGGATT | Myrcene (O. basilicum) |
| | TTAACTATATCCAATCTCTTAATAACAACCATTCTAAAGAAGAACGT | |
| | CACTTAGAGCGTAAAGCAAAACTTATTGAAGAAGTAAAAATGTTAT | |
| | TAGAGCAAGAAATGGCTGCTGTACAACAATTAGAGCTTATTGAAGA | |
| | CCTTAAAAACTTAGGTTTATCTTACTTATTCCAAGATGAAATCAAAA | |
| | TAATCCTTAATTCTATTTACAATCATCATAAATGTTTTCATAATAATC | |
| | ACGAACAATGTATTCACGTTAATAGTGACTTATACTTTGTTGCATTA | |
| | GGCTTCCGTTTATTTCGTCAACATGGTTTCAAAGTTTCTCAAGAGGTT | |
| | TTTGACTGTTTTAAAAACGAAGAAGGATCAGACTTTAGTGCTAACTT | |
| | AGCAGATGATACTAAAGGTTTACTTCAATTATACGAGGCTTCATATT | |
| | TAGTTACAGAAGATGAAGACACATTAGAAATGGCACGTCAATTTTC | |
| | AACTAAAATCTTACAAAAAAAAGTAGAAGAGAAAATGATTGAGAA | |
| | AGAGAACTTATTAAGTTGGACTTTACATAGTTTAGAATTACCACTTC | |
| | ACTGGCGTATTCAACGTTTAGAAGCAAAATGGTTCCTTGATGCTTAT | |
| | GCTAGTCGTCCAGATATGAATCCAATTATTTTTGAATTAGCTAAATT | |
| | AGAGTTTAACATTGCTCAAGCATTACAACAAGAAGAATTAAAAGAT | |
| | TTAAGTAGATGGTGGAATGATACAGGCATTGCTGAAAAATTACCTTT | |
| | TGCTCGTGATAGAATAGTAGAGAGTCATTACTGGGCAATTGGTACTT | |
| | TAGAACCTTATCAATATAGATATCAACGTTCATTAATTGCTAAAATT | |
| | ATTGCTTTAACAACAGTTGTTGATGACGTTTACGACGTATATGGAAC | |
| | TTTAGATGAATTACAGTTATTTACAGACGCTATTCGTCGTTGGGATA | |
| | TTGAATCTATTAATCAATTACCAAGTTATATGCAATTATGCTATTTAG | |
| | CTATTTATAACTTTGTTTCTGAATTAGCATACGATATTTTTCGTGACA | |
| | AAGGATTCAATTCTTTACCTTACCTTCATAAATCATGGTTAGATTTAG | |
| | TAGAAGCATACTTTGTTGAAGCTAAATGGTTTCATGATGGTTATACT | |
| | CCAACTCTTGAAGAATATTTAAATAACTCAAAAATTACTATTATATG | |
| | TCCTGCTATTGTTAGTGAAATCTACTTCGCATTCGCTAATTCAATTGA | |
| | TAAAACAGAAGTTGAATCAATCTACAAATATCACGATATTTTATATT | |
| | TATCAGGAATGCTTGCACGTTTACCAGACGACTTAGGTACTTCATCA | |
| | TTTGAAATGAAAAGAGGTGATGTTGCTAAAGCTATTCAATGTTACAT | |
| | GAAAGAACATAATGCTTCAGAGGAAGAAGCTCGTGAACACATTCGT | |
| | TTCTTAATGCGTGAAGCATGGAAACACATGAATACTGCTGCAGCTGC | |
| | TGATGACTGTCCATTTGAATCTGATTTAGTAGTAGGTGCTGCATCAT | |
| | TAGGTAGAGTTGCAAACTTTGTATATGTTGAAGGTGACGGTTTTGGT | |
| | GTACAACATTCAAAAATACATCAACAAATGGCTGAATTACTTTTTTA | |
| | TCCATATCAAGGTACCGGTGAAAACTTATACTTTCAAGGTAGTGGAG | |
| | GTGGTGGTAGTGACTATAAAGACGATGACGATAAAGGAACCGGTTAA | |
| 101 | ATGGTACCAAGAAGAAGTGCTAATTATCAAGCAAGTATTTGGGATG | Zingiberene |
| | ATAATTTCATTCAAAGTCTTGCATCTCCTTATGCAGGAGAAAAATAT | (O. basilicum) |
| | GCAGAAAAAGCAGAAAAACTTAAAACAGAAGTTAAAACTATGATTG | |
| | ATCAAACAAGAGATGAACTTAAACAATTAGAACTTATTGATAACTT | |
| | ACAACGTTTAGGTATATGTCATCACTTTCAAGACCTTACAAAAAAAA | |
| | TTTTACAAAAAATTTATGGAGAAGAACGTAACGGAGATCACCAACA | |
| | TTACAAAGAAAAAGGCTTACATTTTACAGCATTACGTTTCCGTATTT | |
| | TACGTCAGGACGGTTATCATGTTCCACAAGATGTATTTTCATCATTT | |
| | ATGAATAAAGCTGGTGACTTTGAAGAATCTTTAAGTAAAGACACAA | |
| | AAGGTTTAGTTAGTTTATATGAGGCTTCTTACTTATCAATGGAAGGT | |
| | GAAACTATTTTAGATATGGCAAAAGACTTTTCATCTCACCATTTACA | |
| | TAAAATGGTTGAAGATGCTACTGACAAACGTGTAGCTAATCAAATT | |
| | ATCCATTCTCTTGAAATGCCACTTCACAGACGTGTTCAAAAACTTGA | |
| | AGCAATTTGGTTTATTCAATTCTACGAATGCGGCTCTGATGCTAATC | |
| | CAACTTTAGTAGAATTAGCAAAATTAGATTTCAACATGGTTCAGGCA | |
| | ACATACCAAGAAGAATTAAAACGTTTATCACGTTGGTATGAAGAAA | |
| | CAGGCTTACAAGAGAAACTTTCATTCGCTCGTCACCGTCTTGCTGAA | |
| | GCATTCTTATGGTCTATGGGTATTATTCCAGAAGGACACTTTGGTTA | |
| | TGGTCGTATGCACTTAATGAAAATTGGTGCTTACATTACATTACTTG | |
| | ATGATATTTATGATGTTTATGGTACTTTAGAAGAACTTCAAGTATTA | |
| | ACAGAAATTATTGAACGTTGGGATATTAACTTATTAGATCAATTACC | |
| | TGAATACATGCAAATCTTCTTTTTATACATGTTTAATTCTACAAATGA | |
| | ACTTGCTTATGAAATTTTACGTGATCAAGGTATCAATGTAATATCAA | |
| | ACTTAAAAGGATTATGGGTAGAGTTATCTCAGTGTTACTTTAAAGAA | |
| | GCTACTTGGTTCCATAACGGTTACACACCAACAACTGAAGAATATCT | |
| | TAATGTTGCTTGTATTTCTGCTAGTGGTCCTGTTATTTTATTTTCAGG | |
| | TTACTTTACTACTACTAATCCTATTAATAAACACGAATTACAATCTTT | |
| | AGAACGTCACGCACATTCATTATCTATGATATTACGTTTAGCTGATG | |
| | ATTTAGGTACATCAAGTGATGAAATGAAACGTGGAGATGTACCAAA | |
| | AGCTATTCAATGTTTTATGAATGACACTGGTTGTTGTGAAGAAGAAG | |
| | CACGTCAACACGTAAAAAGATTAATAGATGCTGAATGGAAAAAAAT | |
| | GAACAAAGACATCTTAATGGAGAAACCATTTAAAAATTTTTGTCCAA | |
| | CTGCTATGAATTTAGGTCGTATTTCTATGAGTTTTTATGAACACGGA | |
| | GATGGTTATGGAGGTCCTCACTCTGATACAAAAAAAAAAATGGTAT | |
| | CTTTATTTGTACAACCAATGAATATTACTATTGGTACCGGTGAAAAC | |
| | CTTTATTTTCAAGGTTCTGGTGGTGGCGGTTCAGATTATAAAGATGA | |
| | TGACGACAAAGGAACCGGTTAA | |
| 102 | ATGGTACCAAGACGTTCAGCTAACTATCAACCTAGTATTTGGAACCA | Myrcene (Q. ilex) |
| | CGATTACATTGAATCACTTCGTATCGAATATGTTGGTGAAACATGTA | |
| | CACGTCAAATTAACGTTTTAAAAGAACAAGTTCGTATGATGTTACAC | |
| | AAAGTTGTTAATCCATTAGAACAATTAGAATTAATTGAAATTTTACA | |
| | ACGTTTAGGTTTAAGTTACCATTTCGAAGAAGAAATAAAACGTATTT | |
| | TAGATGGTGTTTACAATAACGATCATGGTGGTGATACATGGAAAGC | |
| | AGAAAACCTTTATGCAACAGCTCTTAAATTCCGTCTTTTACGTCAGC | |
| | ACGGTTATTCTGTTTCTCAAGAAGTTTTCAACTCTTTTAAAGATGAGC | |
| | GTGGCAGTTTCAAAGCATGTTTATGTGAAGATACTAAAGGTATGTTA | |
| | TCACTTTATGAAGCATCTTTCTTTCTTATTGAAGGTGAAAACATTTTA | |
| | GAGGAAGCTAGAGACTTTAGTACAAAACATCTTGAAGAATATGTAA | |
| | AACAAAATAAAGAGAAAAACTTAGCTACTTTAGTTAATCACTCATTA | |
| | GAATTTCCATTACATTGGCGTATGCCTCGTTTAGAAGCTCGTTGGTTC | |
| | ATCAATATCTATCGTCATAATCAAGATGTAAATCCAATCCTTTTAGA | |
| | ATTTGCTGAACTTGACTTCAATATTGTACAAGCTGCTCACCAAGCAG | |
| | ATTTAAAACAAGTATCAACATGGTGGAAATCAACTGGTTTAGTAGA | |
| | AAATCTTTCATTCGCTCGTGATCGTCCTGTAGAAAACTTCTTTTGGAC | |
| | AGTTGGTCTTATTTTCCAACCACAATTCGGTTATTGTCGTAGAATGTT | |
| | TACTAAAGTATTCGCATTAATTACTACAATTGATGACGTATATGATG | |
| | TATATGGTACTTTAGATGAATTAGAACTTTTCACAGACGTTGTTGAA | |
| | AGATGGGATATTAATGCAATGGATCAATTACCTGATTATATGAAAAT | |
| | TTGCTTTTTAACATTACACAATAGTGTTAACGAAATGGCATTAGACA | |
| | CTATGAAAGAACAACGTTTTCACATCATTAAATACCTTAAAAAAGCA | |
| | TGGGTTGATCTTTGTCGTTATTACTTAGTTGAAGCTAAATGGTATAGT | |
| | AATAAATATAGACCTTCTTTACAAGAATACATTGAAAATGCATGGAT | |
| | TTCAATTGGTGCTCCAACTATTTTAGTTCATGCATATTTCTTCGTTAC | |
| | AAATCCAATTACAAAAGAAGCATTAGACTGTTTAGAAGAATATCCA | |
| | AACATTATTCGTTGGAGTAGTATTATTGCACGTTTAGCTGATGATTT | |
| | AGGTACTTCAACAGACGAATTAAAACGTGGTGACGTACCAAAAGCA | |
| | ATTCAATGTTATATGAATGAAACAGGTGCTTCAGAAGAAGGTGCTC | |
| | GTGAGTACATTAAATACTTAATTTCTGCTACTTGGAAAAAAATGAAC | |
| | AAAGATAGAGCAGCATCAAGTCCATTTTCACATATCTTCATTGAAAT | |
| | TGCTCTTAATTTAGCACGTATGGCACAATGTTTATATCAACACGGTG | |
| | ACGGCCACGGTTTAGGTAACCGTGAAACAAAAGATCGTATACTTTC | |
| | ATTACTTATTCAACCAATTCCATTAAACAAAGATGGTACCGGTGAGA | |
| | ACTTATACTTTCAAGGCTCAGGTGGTGGTGGTTCTGATTACAAAGAT | |
| | GATGATGATAAAGGAACCGGTTAA | |
| 103 | ATGGTACCAAGAAGAATTGGAGACTATCACTCAAACTTATGGAATG | Myrcene (P. abies) |
| | ATGACTTCATTCAATCATTAACAACACCATACGGTGCTCCATCATAT | |
| | ATTGAACGTGCTGATAGATTAATATCTGAAGTAAAAGAAATGTTTAA | |
| | TAGAATGTGTATGGAAGATGGTGAGTTAATGTCTCCATTAAATGATC | |
| | TTATTCAAAGATTATGGACTGTTGATAGTGTTGAACGTTTAGGTATA | |
| | GATCGTCACTTCAAAAATGAAATAAAAGCTAGTTTAGATTATGTATA | |
| | CTCATACTGGAACGAAAAAGGTATCGGTTGTGGTCGTCAATCAGTA | |
| | GTTACAGATTTAAACTCTACTGCTCTTGGATTAAGAATTTTACGTCA | |
| | ACATGGTTACACAGTTTCAAGTGAAGTTTTAAAAGTTTTTGAAGAAG | |
| | AAAACGGTCAATTTGCTTGTTCACCTTCACAGACTGAGGGCGAAATT | |
| | CGTTCATTCTTAAACTTATATCGTGCTTCATTAATTGCTTTTCCTGGT | |
| | GAAAAAGTAATGGAAGAAGCTCAAATCTTTTCTAGTCGTTACTTAAA | |
| | AGAAGCAGTTCAGAAAATTCCAGTTTCAGGTTTATCTCGTGAAATAG | |
| | GCGATGTTTTAGAATATGGTTGGCACACAAACTTACCTCGTTGGGAA | |
| | GCTCGTAACTATATGGACGTATTCGGTCAAGACACAAATACATCATT | |
| | CAACAAAAACAAAATGCAATATATGAATACAGAGAAAATTCTTCAA | |
| | TTAGTAAAATTAGAGTTTAATATCTTTCATTCATTACAACAACGTGA | |
| | ATTACAATGTTTATTACGTTGGTGGAAAGAAAGTGGTCTTCCACAAT | |
| | TAACATTTGCACGTCACCGTCACGTTGAATTTTACACTTTAGCTTCTT | |
| | GTATTGCATGTGAACCAAAACACAGTGCATTTCGTTTAGGTTTTGCA | |
| | AAAATGTGTCACTTAGTAACAGTTTTAGATGATGTATATGACACATT | |
| | TGGCAAAATGGATGAATTAGAACTTTTTACTGCAGCTGTTAAACGTT | |
| | GGGACTTATCAGAAACTGAGCGTTTACCTGAGTATATGAAAGGTTTA | |
| | TATGTTGTAGTTTTCGAGACTGTTAATGAATTAGCACAAGAAGCAGA | |
| | GAAAACTCAAGGACGTAATACATTAAATTACGTTCGTAAAGCATGG | |
| | GAAGCATACTTCGATAGTTATATGAAAGAAGCAGAATGGATCTCAA | |
| | CAGGCTATTTACCAACATTCGAAGAGTATTGTGAAAACGGTAAAGT | |
| | ATCAAGTGCATATAGAGTTGCTGCACTTCAACCTATTTTAACATTAG | |
| | ATGTACAACTTCCAGATGACATCTTAAAAGGTATTGATTTTCCATCT | |
| | CGTTTCAATGATTTAGCATCTTCATTTCTTCGTTTACGTGGAGATACT | |
| | AGATGTTACGAGGCTGATCGTGCTCGTGGTGAAGAAGCAAGTTGTA | |
| | TTTCTTGTTACATGAAAGACAATCCAGGTTCAACTGAAGAAGATGCA | |
| | TTAAATCACATTAATGCTATGATAAATGATATTATTCGTGAATTAAA | |
| | CTGGGAATTTCTTAAACCAGACTCAAATATCCCAATGCCAGCTCGTA | |
| | AACATGCTTTCGATATTACAAGAGCTTTACATCACTTATATATTTATC | |
| | GTGACGGTTTTTCTGTTGCTAACAAAGAGACTAAAAATCTTGTTGAG | |
| | AAAACTTTATTAGAATCAATGTTATTCGGTACCGGTGAGAACCTTTA | |
| | TTTTCAAGGTTCAGGTGGTGGTGGTTCAGATTATAAAGACGATGATG | |
| | ATAAAGGAACCGGTTAA | |
| 104 | ATGGTACCAAGAAGATCAGCTAATTATCAACCTAGTCGTTGGGATCA | Myrcene, |
| | TCATCACCTTTTAAGTGTAGAAAACAAATTCGCTAAAGATAAACGTG | ocimene (A. thalania) |
| | TAAGAGAACGTGACTTACTTAAAGAAAAAGTTCGTAAAATGTTAAA | |
| | TGACGAACAGAAAACTTACTTAGATCAATTAGAATTTATTGACGATC | |
| | TTCAAAAATTAGGTGTTAGTTATCACTTCGAAGCAGAAATAGATAAT | |
| | ATACTTACAAGTTCATACAAAAAAGATCGTACAAATATACAAGAAA | |
| | GTGATTTACACGCAACTGCATTAGAGTTTCGTCTTTTTCGTCAACAC | |
| | GGTTTTAACGTTTCAGAAGATGTATTTGATGTATTTATGGAAAATTG | |
| | TGGTAAATTCGACCGTGATGACATTTATGGTTTAATTTCATTATATG | |
| | AAGCTAGTTATCTTTCTACTAAACTTGACAAAAATCTTCAAATCTTT | |
| | ATCCGTCCATTTGCTACTCAACAATTACGTGATTTTGTAGATACTCAC | |
| | AGTAATGAAGATTTCGGTTCATGTGATATGGTAGAAATAGTTGTTCA | |
| | AGCATTAGACATGCCATACTATTGGCAAATGCGTCGTTTATCTACAC | |
| | GTTGGTATATTGATGTTTATGGTAAAAGACAAAATTACAAAAACTTA | |
| | GTAGTTGTTGAATTTGCAAAAATTGATTTCAATATTGTTCAAGCTATT | |
| | CACCAGGAAGAACTTAAAAATGTATCATCTTGGTGGATGGAAACTG | |
| | GTTTAGGTAAACAACTTTATTTTGCTCGTGATCGTATTGTAGAGAAC | |
| | TATTTTTGGACAATTGGTCAAATTCAAGAACCTCAATATGGATATGT | |
| | TAGACAAACAATGACTAAAATCAATGCTTTATTAACAACAATTGATG | |
| | ATATTTATGATATATACGGTACATTAGAAGAATTACAGTTATTCACA | |
| | GTTGCATTTGAGAATTGGGACATAAATCGTTTAGACGAATTACCAGA | |
| | ATATATGCGTTTATGTTTCTTAGTTATCTATAACGAAGTAAATAGTAT | |
| | AGCATGTGAAATTCTTAGAACAAAAAATATTAACGTTATTCCTTTCT | |
| | TAAAAAAATCTTGGACTGATGTAAGTAAAGCATACTTAGTTGAAGCT | |
| | AAATGGTATAAATCAGGCCATAAACCAAATTTAGAAGAGTATATGC | |
| | AAAATGCACGTATTTCTATTTCTTCACCAACAATCTTTGTTCACTTTT | |
| | ATTGTGTATTTTCAGACCAATTATCTATTCAAGTTTTAGAAACTTTAT | |
| | CACAACACCAACAAAATGTTGTAAGATGTAGTTCTTCTGTTTTCCGT | |
| | TTAGCTAATGACTTAGTAACTTCTCCAGATGAATTAGCTAGAGGTGA | |
| | TGTTTGTAAATCAATTCAATGTTATATGTCAGAAACTGGTGCAAGTG | |
| | AAGATAAAGCTAGATCACACGTTCGTCAAATGATTAATGATTTATGG | |
| | GACGAAATGAATTACGAGAAAATGGCACATTCAAGTAGTATCTTAC | |
| | ATCATGATTTTATGGAGACAGTAATCAATTTAGCTAGAATGTCTCAA | |
| | TGTATGTACCAATATGGTGACGGACACGGTTCTCCAGAAAAAGCTA | |
| | AAATTGTAGATCGTGTAATGAGTTTACTTTTCAACCCTATTCCTTTAG | |
| | ATGGTACCGGTGAGAATTTATATTTTCAAGGCTCTGGAGGTGGTGGT | |
| | TCAGATTATAAAGATGATGACGACAAAGGAACCGGTTAA | |
| 105 | ATGGTACCAAGAAGAAGTGCAAACTATCAACCTTCATTATGGCAAC | Myrcene, |
| | ATGAATACTTATTATCATTAGGCAACACTTATGTTAAAGAAGATAAT | ocimene (A. thalania) |
| | GTTGAAAGAGTAACTCTTTTAAAACAAGAAGTTTCTAAAATGTTAAA | |
| | CGAAACAGAAGGTTTACTTGAACAACTTGAATTAATTGACACTTTAC | |
| | AAAGATTAGGTGTTTCTTATCATTTTGAACAGGAGATTAAAAAAACA | |
| | TTAACTAATGTTCATGTTAAAAACGTACGTGCTCATAAAAATCGTAT | |
| | TGATCGTAACCGTTGGGGCGATTTATATGCAACTGCATTAGAATTTC | |
| | GTTTATTACGTCAACATGGTTTTTCTATTGCTCAAGACGTTTTTGATG | |
| | GTAATATTGGTGTTGACTTAGACGACAAAGACATTAAAGGTATTTTA | |
| | AGTTTATACGAAGCTAGTTACTTATCAACACGTATTGATACAAAACT | |
| | TAAAGAATCAATCTATTACACAACAAAACGTTTAAGAAAATTCGTA | |
| | GAGGTAAACAAAAACGAAACTAAAAGTTACACTCTTCGTCGTATGG | |
| | TTATTCACGCACTTGAGATGCCTTATCACCGTCGTGTTGGTCGTCTTG | |
| | AAGCTCGTTGGTATATCGAGGTATATGGAGAAAGACACGACATGAA | |
| | TCCTATTTTATTAGAATTAGCTAAATTAGATTTTAACTTTGTTCAGGC | |
| | TATCCACCAAGACGAATTAAAATCATTATCTAGTTGGTGGTCTAAAA | |
| | CAGGATTAACAAAACATTTAGACTTTGTTCGTGATCGTATTACAGAG | |
| | GGTTACTTCAGTAGTGTAGGTGTTATGTATGAACCAGAATTTGCATA | |
| | TCATCGTCAAATGCTTACAAAAGTATTTATGCTTATTACAACTATTG | |
| | ATGACATCTATGACATTTACGGTACACTTGAAGAATTACAATTATTC | |
| | ACAACTATCGTTGAAAAATGGGATGTTAATCGTTTAGAAGAACTTCC | |
| | TAACTATATGAAATTATGCTTCTTATGTTTAGTTAACGAAATAAATC | |
| | AAATTGGATATTTTGTATTAAGAGATAAAGGTTTTAATGTAATTCCT | |
| | TATCTTAAAGAGTCTTGGGCTGACATGTGTACTACATTTCTTAAAGA | |
| | AGCTAAATGGTACAAATCAGGTTATAAACCAAATTTTGAAGAGTAT | |
| | ATGCAAAATGGCTGGATTTCATCATCAGTTCCAACTATTCTTTTACA | |
| | CTTATTTTGTTTATTAAGTGACCAAACTTTAGACATTCTTGGTTCTTA | |
| | TAATCACAGTGTTGTTCGTAGTTCAGCAACAATTTTACGTCTTGCAA | |
| | ATGATTTAGCTACTTCTTCAGAAGAATTAGCAAGAGGAGATACAAT | |
| | GAAATCAGTTCAATGTCACATGCATGAAACTGGTGCTTCAGAAGCTG | |
| | AATCAAGAGCTTACATTCAAGGTATTATTGGCGTAGCTTGGGATGAC | |
| | CTTAATATGGAGAAAAAATCATGTCGTTTACACCAGGGATTCTTAGA | |
| | AGCAGCAGCAAATTTAGGACGTGTAGCACAATGCGTATATCAATAT | |
| | GGAGACGGTCACGGTTGTCCAGATAAAGCAAAAACAGTAAATCATG | |
| | TTCGTAGTTTATTAGTTCACCCATTACCATTAAACGGTACCGGTGAA | |
| | AACCTTTATTTTCAAGGTAGTGGTGGAGGTGGTTCTGATTATAAAGA | |
| | CGACGATGACAAAGGAACCGGTTAA | |
| 106 | ATGGTACCAGCTTCTCCACCTGCTCATCGTTCATCTAAAGCAGCAGA | Sesquiterpene |
| | CGAAGAGTTACCAAAAGCATCTTCTACATTCCATCCATCTCTTTGGG | (Z. mays; |
| | GTTCATTTTTCTTAACATATCAGCCACCTACAGCTCCACAACGTGCA | B73) |
| | AATATGAAAGAACGTGCTGAAGTTCTTCGTGAACGTGTTCGTAAAGT | |
| | ATTAAAAGGTTCAACAACAGATCAATTACCTGAAACAGTTAACTTA | |
| | ATTCTTACATTACAAAGACTTGGTTTAGGTTATTACTATGAAAATGA | |
| | AATTGACAAATTACTTCATCAAATTTACTCTAATTCAGATTATAACG | |
| | TAAAAGACTTAAACTTAGTTTCTCAACGTTTTTACTTACTTCGTAAAA | |
| | ACGGTTATGACGTACCTTCTGATGTTTTCTTATCTTTTAAAACTGAAG | |
| | AAGGTGGTTTCGCTTGTGCTGCAGCTGACACACGTTCACTTTTAAGT | |
| | TTATACAATGCTGCTTACCTTCGTAAACATGGTGAAGAAGTATTAGA | |
| | TGAAGCAATTTCATCAACACGTTTAAGATTACAAGACTTATTAGGTC | |
| | GTTTATTACCTGAATCACCATTCGCTAAAGAAGTATCAAGTTCACTT | |
| | CGTACACCTTTATTCCGTCGTGTAGGTATTTTAGAAGCTCGTAACTAT | |
| | ATTCCAATCTATGAAACTGAAGCTACAAGAAATGAAGCTGTATTAG | |
| | AGCTTGCTAAACTTAACTTCAATTTACAACAGCTTGATTTCTGTGAA | |
| | GAATTAAAACATTGTAGTGCATGGTGGAATGAGATGATTGCTAAAA | |
| | GTAAATTAACTTTTGTACGTGACCGTATAGTTGAAGAATACTTTTGG | |
| | ATGAATGGTGCATGTTATGATCCACCATATTCATTAAGTCGTATTAT | |
| | TCTTACAAAAATCACTGGTTTAATTACTATTATTGATGATATGTTCGA | |
| | TACTCATGGTACAACAGAGGATTGCATGAAATTCGCAGAAGCATTT | |
| | GGTCGTTGGGATGAATCAGCAATTCATCTTCTTCCAGAATACATGAA | |
| | AGATTTTTACATTTTAATGTTAGAAACTTTCCAGTCATTTGAAGATGC | |
| | ACTTGGTCCAGAAAAATCATACCGTGTATTATACTTAAAACAAGCAA | |
| | TGGAACGTTTAGTAGAGTTATATTCTAAAGAAATCAAATGGCGTGAT | |
| | GACGATTATGTTCCAACAATGTCAGAACATTTACAAGTTAGTGCTGA | |
| | AACAATTGCTACAATTGCTTTAACTTGCTCTGCTTATGCTGGTATGG | |
| | GTGATATGTCTATTCGTAAAGAAACATTTGAATGGGCATTATCTTTC | |
| | CCTCAATTCATTAGAACTTTTGGTTCATTTGTACGTTTATCAAATGAT | |
| | GTTGTATCAACAAAACGTGAACAAACTAAAGATCATTCACCTTCAAC | |
| | AGTTCACTGTTATATGAAAGAACACGGTACAACTATGGACGATGCTT | |
| | GTGAAAAAATCAAAGAATTAATTGAGGACTCATGGAAAGACATGTT | |
| | AGAACAATCTTTAGCTCTTAAAGGCTTACCTAAAGTAGTACCTCAAT | |
| | TAGTTTTTGATTTCTCTCGTACTACAGATAACATGTATCGTGACCGTG | |
| | ATGCTTTAACATCATCAGAAGCATTAAAAGAAATGATACAGTTATTA | |
| | TTCGTAGAACCTATACCTGAAGGTACCGGTGAGAATCTTTATTTTCA | |
| | AGGATCAGGTGGTGGAGGCTCAGATTACAAAGATGACGACGATAAA | |
| | GGAACCGGTTAA | |
| 107 | ATGGTACCAGAGGCTTTAGGAAATTTTGATTATGAGAGTTATACTAA | Sesquiterpene |
| | TTTTACAAAATTACCATCATCACAATGGGGTGATCAATTCCTTAAAT | (A. thalania) |
| | TTTCTATAGCAGATTCTGACTTCGATGTATTAGAAAGAGAAATAGAA | |
| | GTATTAAAACCAAAAGTAAGAGAGAACATTTTTGTTTCATCAAGTAC | |
| | TGATAAAGATGCAATGAAAAAAACAATTTTAAGTATTCATTTCTTAG | |
| | ATAGTTTAGGTTTATCTTATCACTTCGAAAAAGAAATAGAGGAGAGT | |
| | TTAAAACATGCTTTCGAGAAAATTGAAGACCTTATTGCTGATGAAAA | |
| | TAAACTTCATACAATAAGTACAATTTTCCGTGTATTCCGTACATACG | |
| | GCTATTATATGTCTTCTGATGTATTCAAAATTTTCAAAGGAGACGAT | |
| | GGTAAATTCAAAGAAAGTTTAATTGAAGACGTTAAAGGTATGCTTTC | |
| | TTTTTATGAAGCTGTTCATTTTGGAACAACTACTGATCACATTTTAGA | |
| | CGAAGCTCTTAGTTTTACATTAAACCACTTAGAGTCACTTGCAACAG | |
| | GCCGTCGTGCATCACCACCACATATTAGTAAATTAATCCAAAATGCT | |
| | TTACATATTCCTCAACATCGTAACATCCAGGCATTAGTAGCTCGTGA | |
| | ATACATTAGTTTTTACGAACACGAAGAAGATCACGATGAAACATTAT | |
| | TAAAATTAGCTAAATTAAACTTTAAATTCTTACAACTTCACTATTTTC | |
| | AAGAATTAAAAACAATTACAATGTGGTGGACTAAATTAGATCATAC | |
| | ATCTAATTTACCACCAAATTTTCGTGAACGTACAGTTGAAACATGGT | |
| | TTGCAGCTTTAATGATGTATTTCGAACCACAATTTAGTTTAGGTCGT | |
| | ATTATGAGTGCAAAATTATATTTAGTAATTACTTTCTTAGATGACGC | |
| | ATGTGATACATACGGATCAATATCTGAAGTAGAGTCATTAGCTGATT | |
| | GTTTAGAACGTTGGGACCCAGATTATATGGAAAATTTACAAGGTCAC | |
| | ATGAAAACAGCATTCAAATTCGTTATGTATTTATTCAAAGAATACGA | |
| | AGAAATTTTACGTTCACAAGGCCGTTCATTCGTATTAGAGAAAATGA | |
| | TTGAGGAGTTTAAAATTATCGCACGTAAAAACTTAGAACTTGTAAAA | |
| | TGGGCTCGTGGTGGTCACGTTCCTTCTTTTGACGAATATATAGAGAG | |
| | TGGTGGTGCTGAGATTGGTACTTATGCTACAATCGCTTGTTCAATTA | |
| | TGGGTCTTGGTGAAATTGGTAAAAAAGAAGCATTTGAGTGGTTAATC | |
| | TCTCGTCCTAAACTTGTTCGTATTTTAGGTGCTAAAACACGTTTAATG | |
| | GATGATATCGCAGACTTTGAAGAAGACATGGAAAAAGGCTATACAG | |
| | CTAATGCACTTAACTATTATATGAATGAACACGGAGTAACTAAAGA | |
| | AGAAGCTAGTCGTGAACTTGAGAAAATGAATGGTGATATGAACAAA | |
| | ATTGTAAACGAAGAATGTCTTAAAATTACAACTATGCCACGTCGTAT | |
| | CTTAATGCAAAGTGTTAACTACGCTCGTAGTTTAGATGTATTATACA | |
| | CAGCTGATGATGTATATAACCACCGTGAAGGCAAACTTAAAGAATA | |
| | TATGAGATTACTTTTAGTAGATCCAATTTTACTTGGTACCGGTGAAA | |
| | ATCTTTATTTTCAAGGTTCAGGTGGTGGTGGTTCTGATTATAAAGAT | |
| | GATGACGATAAAGGAACCGGTTAA | |
| 108 | ATGGTACCAGAGAGTCAAACAACATTCAAATACGAATCATTAGCAT | Sesquiterpene |
| | TTACAAAACTTAGTCACTGTCAATGGACAGACTATTTTCTTAGTGTT | (A. thalania) |
| | CCAATTGATGAAAGTGAATTAGATGTTATTACTCGTGAAATTGATAT | |
| | TCTTAAACCAGAAGTTATGGAGTTATTAAGTAGTCAAGGAGATGAT | |
| | GAAACAAGTAAAAGAAAAGTTCTTCTTATTCAGTTATTACTTTCTTT | |
| | AGGTTTAGCATTCCACTTTGAAAATGAGATTAAAAACATACTTGAAC | |
| | ACGCATTTCGTAAAATAGATGATATAACTGGTGACGAAAAAGACTT | |
| | ATCAACAATTAGTATTATGTTCCGTGTTTTCCGTACTTATGGACACA | |
| | ATCTTCCAAGTAGTGTTTTTAAACGTTTCACAGGTGATGATGGTAAA | |
| | TTTCAGCAAAGTTTAACAGAAGACGCAAAAGGTATTTTAAGTTTATA | |
| | TGAAGCTGCACATTTAGGTACTACTACAGATTACATTTTAGATGAAG | |
| | CTCTTAAATTCACATCTAGTCACTTAAAAAGTTTACTTGCTGGTGGT | |
| | ACATGTCGTCCTCACATCTTACGTTTAATCCGTAATACATTATACTTA | |
| | CCACAACGTTGGAACATGGAAGCTGTTATCGCTCGTGAATACATATC | |
| | ATTTTACGAGCAGGAAGAAGATCACGATAAAATGCTTTTACGTCTTG | |
| | CAAAACTTAACTTTAAACTTCTTCAATTACACTACATTAAAGAGCTT | |
| | AAAAGTTTCATTAAATGGTGGATGGAACTTGGTTTAACTTCTAAATG | |
| | GCCTTCTCAATTTCGTGAACGTATTGTTGAAGCATGGTTAGCTGGAT | |
| | TAATGATGTATTTTGAACCACAGTTCTCAGGTGGTCGTGTTATTGCT | |
| | GCAAAATTCAACTATTTACTTACAATATTAGACGACGCATGTGACCA | |
| | CTATTTTTCTATTCACGAATTAACACGTTTAGTTGCATGTGTAGAACG | |
| | TTGGTCACCAGATGGTATTGACACATTAGAAGATATTTCACGTTCTG | |
| | TATTCAAATTAATGTTAGATGTTTTCGACGATATTGGTAAAGGTGTA | |
| | CGTTCAGAAGGTTCTAGTTACCACTTAAAAGAAATGTTAGAGGAATT | |
| | AAACACTTTAGTTCGTGCTAATTTAGATTTAGTTAAATGGGCTCGTG | |
| | GAATACAAACAGCTGGTAAAGAGGCTTATGAATGGGTTCGTTCACG | |
| | TCCACGTTTAATCAAATCTTTAGCAGCTAAAGGTAGACTTATGGATG | |
| | ATATTACAGACTTTGACTCAGATATGAGTAATGGATTCGCAGCTAAT | |
| | GCTATTAACTACTATATGAAACAATTTGTTGTTACAAAAGAAGAAGC | |
| | TATTCTTGAATGTCAACGTATGATTGTAGACATTAACAAAACTATTA | |
| | ATGAAGAGTTATTAAAAACTACTTCAGTTCCAGGTCGTGTATTAAAA | |
| | CAAGCTCTTAACTTTGGCCGTTTATTAGAATTATTATATACAAAATCT | |
| | GACGATATTTACAATTGTTCTGAAGGCAAACTTAAAGAATACATTGT | |
| | AACTCTTTTAATTGATCCTATAAGACTTGGTACCGGTGAAAACTTAT | |
| | ACTTTCAAGGTTCAGGCGGTGGTGGTAGTGATTACAAAGATGATGAT | |
| | GACAAAGGAACCGGTTAA | |
| 109 | ATGGTACCAGAGAGTCAAACAAAATTCGACTACGAATCATTAGCTTT | Sesquiterpene |
| | TACAAAATTATCACATTCACAATGGACTGATTACTTTTTATCAGTAC | (A. thalania) |
| | CTATAGACGACTCTGAACTTGACGCAATTACTCGTGAAATCGACATT | |
| | ATCAAACCTGAAGTTCGTAAATTACTTTCAAGTAAAGGTGATGATGA | |
| | AACTTCTAAACGTAAAGTATTACTTATCCAAAGTTTATTATCATTAG | |
| | GTTTAGCATTTCATTTTGAAAACGAAATTAAAGATATTTTAGAAGAT | |
| | GCATTTAGACGTATTGATGACATTACAGGTGATGAAAACGACTTAA | |
| | GTACTATTAGTATTATGTTCCGTGTATTCCGTACATACGGTCACAATT | |
| | TACCAAGTAGTGTTTTTAAACGTTTCACTGGTGATGACGGTAAATTT | |
| | GAACGTTCTTTAACTGAAGATGCTAAAGGAATTTTATCATTATATGA | |
| | AGCTGCACATTTAGGAACAACTACTGATTATATTCTTGATGAAGCAT | |
| | TAGAATTTACTTCATCACACTTAAAATCTTTACTTGTTGGTGGTATGT | |
| | GTCGTCCACATATTTTACGTCTTATTAGAAATACTTTATATCTTCCAC | |
| | AACGTTGGAATATGGAAGCAGTAATTGCAAGAGAATACATTAGTTT | |
| | TTATGAACAAGAAGAAGATCACGATAAAATGTTACTTCGTTTAGCTA | |
| | AATTAAATTTCAAATTACTTCAATTACACTACATTAAAGAGTTAAAA | |
| | ACATTCATTAAATGGTGGATGGAATTAGGACTTACATCAAAATGGCC | |
| | TTCTCAATTTCGTGAACGTATTGTTGAAGCATGGTTAGCTGGTCTTAT | |
| | GATGTATTTTGAACCACAGTTTTCTGGAGGTCGTGTAATAGCTGCTA | |
| | AATTCAATTACTTATTAACAATTTTAGATGATGCATGTGATCACTATT | |
| | TCTCAATTCCAGAATTAACTCGTTTAGTTGATTGCGTAGAAAGATGG | |
| | AATCATGATGGTATACATACTTTAGAAGACATCTCACGTATCATCTT | |
| | TAAACTTGCATTAGATGTATTTGATGATATTGGTCGTGGTGTTCGTTC | |
| | TAAAGGTTGTTCTTATTACTTAAAAGAAATGTTAGAAGAGTTAAAAA | |
| | TCTTAGTTCGTGCAAACTTAGATTTAGTTAAATGGGCTCGTGGTAAT | |
| | CAATTACCTAGTTTTGAAGAACACGTTGAGGTAGGTGGTATTGCTCT | |
| | TACAACATACGCAACTTTAATGTACTCTTTTGTTGGCATGGGTGAAG | |
| | CAGTAGGTAAAGAAGCATACGAATGGGTACGTTCTCGTCCACGTTTA | |
| | ATCAAAAGTTTAGCAGCAAAAGGTCGTCTTATGGACGATATTACTGA | |
| | TTTCGAAGTAAAAATTATCAACTTATTTTTCGACCTTCTTTTATTTGT | |
| | ATTCGGTACCGGTGAAAACTTATATTTCCAGGGTAGTGGTGGAGGA | |
| | GGTTCAGACTACAAAGATGACGATGACAAAGGAACCGGTTAA | |
| 110 | ATGGTACCAGCAGCTTTCACAGCAAATGCAGTTGACATGCGTCCACC | Curcumene |
| | AGTTATTACAATTCACCCACGTTCAAAAGATATTTTCTCTCAATTTTC | (P. cablin) |
| | TTTAGATGATAAATTACAAAAACAATACGCTCAAGGAATCGAAGCT | |
| | CTTAAAGAAGAAGCTCGTTCTATGCTTATGGCTGCAAAATCTGCTAA | |
| | AGTAATGATCTTAATTGATACACTTGAACGTTTAGGATTAGGTTATC | |
| | ACTTTGAAAAAGAAATTGAAGAGAAATTAGAAGCTATTTACAAAAA | |
| | AGAGGATGGTGACGATTATGATCTTTTTACAACTGCTTTAAGATTCC | |
| | GTTTACTTAGACAACACCAACGTCGTGTACCATGTTCTGTTTTTGAC | |
| | AAATTTATGAATAAAGAGGGTAAATTCGAAGAAGAACCATTAATTT | |
| | CAGATGTTGAAGGTCTTCTTTCATTATATGACGCTGCTTATTTACAGA | |
| | TTCACGGTGAACACATTTTACAAGAGGCTTTAATTTTCACTACACAT | |
| | CATTTAACTCGTATTGAACCACAATTAGATGATCACTCTCCTTTAAA | |
| | ATTAAAATTAAACCGTGCTTTAGAATTTCCTTTTTACAGAGAAATCC | |
| | CTATAATCTATGCACATTTTTACATTTCAGTATATGAACGTGACGATT | |
| | CTCGTGATGAAGTATTATTAAAAATGGCTAAATTATCTTATAATTTC | |
| | TTACAAAACTTATACAAAAAAGAATTAAGTCAACTTTCTCGTTGGTG | |
| | GAACAAATTAGAACTTATTCCTAATTTACCTTATATTCGTGATTCTGT | |
| | AGCTGGAGCTTATTTATGGGCTGTTGCTTTATATTTCGAACCTCAATA | |
| | TTCAGACGTTCGTATGGCAATTGCTAAACTTATCCAAATTGCAGCAG | |
| | CTGTAGATGATACTTACGATAATTATGCTACTATACGTGAAGCTCAA | |
| | TTATTAACAGAAGCATTAGAACGTTTAAATGTACACGAAATTGACAC | |
| | ATTACCAGATTATATGAAAATTGTTTATCGTTTTGTAATGTCATGGA | |
| | GTGAAGATTTCGAACGTGATGCTACAATTAAAGAACAGATGTTAGC | |
| | TACACCTTATTTCAAAGCTGAAATGAAAAAACTTGGTCGTGCTTATA | |
| | ATCAAGAACTTAAATGGGTTATGGAACGTCAATTACCTAGTTTCGAA | |
| | GAATACATGAAAAACTCTGAAATCACTTCTGGTGTTTACATTATGTT | |
| | TACTGTAATTAGTCCTTACTTAAATAGTGCAACACAAAAAAACATTG | |
| | ACTGGTTATTATCACAACCTCGTTTAGCATCTTCAACTGCAATTGTTA | |
| | TGCGTTGTTGTAATGATTTAGGCTCTAATCAACGTGAATCTAAAGGA | |
| | GGAGAAGTTATGACATCTTTAGATTGCTATATGAAACAACACGGTGC | |
| | TAGTAAACAAGAAACAATTTCTAAATTCAAACTTATTATCGAAGATG | |
| | AATGGAAAAACTTAAATGAAGAATGGGCTGCAACAACATGTCTTCC | |
| | AAAAGTTATGGTAGAAATTTTTCGTAACTATGCACGTATTGCAGGCT | |
| | TTTGCTACAAAAATAACGGTGATGCTTATACATCTCCAAAAATTGTA | |
| | CAACAATGTTTTGACGCTTTATTTGTAAATCCATTAAGAATTGGTAC | |
| | CGGTGAGAATTTATACTTTCAAGGCTCAGGTGGAGGTGGTAGTGATT | |
| | ATAAAGATGATGATGATAAAGGAACCGGTTAA | |
| 111 | ATGGTACCAGAATTTAGAGTTCATTTACAGGCTGATAATGAACAGA | Farnesene |
| | AAATATTCCAGAACCAAATGAAACCTGAACCTGAAGCATCATATCTT | (M. domestica) |
| | ATTAATCAACGTAGATCAGCTAATTACAAACCTAATATTTGGAAAAA | |
| | TGACTTTTTAGATCAAAGTTTAATTAGTAAATACGACGGTGATGAAT | |
| | ATCGTAAATTAAGTGAGAAATTAATCGAGGAAGTAAAAATTTATAT | |
| | ATCTGCTGAGACAATGGACTTAGTAGCTAAATTAGAACTTATTGATT | |
| | CTGTTCGTAAATTAGGTTTAGCTAATCTTTTTGAAAAAGAAATTAAA | |
| | GAAGCATTAGATTCTATCGCAGCTATTGAGTCAGATAATTTAGGTAC | |
| | TCGTGATGACTTATATGGTACTGCTTTACACTTTAAAATTTTACGTCA | |
| | ACATGGTTATAAAGTTTCTCAAGATATTTTTGGTCGTTTCATGGATG | |
| | AAAAAGGTACATTAGAAAATCATCACTTCGCTCACTTAAAAGGTAT | |
| | GTTAGAATTATTTGAAGCATCTAATTTAGGTTTTGAAGGTGAAGATA | |
| | TTTTAGATGAAGCAAAAGCATCACTTACATTAGCTCTTCGTGATAGT | |
| | GGTCATATTTGTTATCCAGATTCTAACTTAAGTCGTGATGTAGTACA | |
| | CTCATTAGAATTACCTAGTCACCGTCGTGTTCAATGGTTTGATGTTA | |
| | AATGGCAAATTAATGCTTATGAAAAAGATATTTGTAGAGTTAATGCA | |
| | ACTCTTTTAGAATTAGCAAAATTAAATTTTAACGTAGTACAAGCACA | |
| | ACTTCAAAAAAACTTACGTGAAGCATCTCGTTGGTGGGCTAACTTAG | |
| | GTTTCGCTGATAACTTAAAATTCGCTCGTGATCGTTTAGTTGAATGTT | |
| | TTTCTTGCGCAGTAGGCGTAGCATTTGAACCTGAACACTCTTCTTTTC | |
| | GTATCTGTTTAACAAAAGTTATTAATTTAGTTTTAATAATTGATGAC | |
| | GTATACGACATATATGGAAGTGAAGAAGAATTAAAACACTTTACAA | |
| | ATGCTGTTGATCGTTGGGATTCTCGTGAAACAGAACAATTACCAGAA | |
| | TGTATGAAAATGTGCTTTCAAGTTTTATACAATACTACATGTGAAAT | |
| | TGCTCGTGAAATTGAAGAAGAAAATGGATGGAATCAAGTTTTACCT | |
| | CAATTAACTAAAGTATGGGCTGATTTTTGTAAAGCATTATTAGTAGA | |
| | AGCTGAATGGTACAATAAAAGTCACATCCCAACTTTAGAAGAATAT | |
| | CTTCGTAATGGCTGTATTTCATCAAGTGTTTCTGTATTATTAGTACAT | |
| | TCTTTCTTTAGTATTACACATGAAGGTACAAAAGAAATGGCAGATTT | |
| | CTTACACAAAAACGAAGACTTATTATACAACATCTCATTAATTGTAC | |
| | GTTTAAACAACGACTTAGGTACAAGTGCAGCTGAACAAGAACGTGG | |
| | TGATTCACCATCATCTATTGTATGTTACATGCGTGAAGTTAATGCTA | |
| | GTGAAGAAACAGCTCGTAAAAATATAAAAGGAATGATCGACAATGC | |
| | TTGGAAAAAAGTTAATGGTAAATGTTTTACAACTAATCAAGTTCCTT | |
| | TTCTTTCTTCTTTTATGAATAACGCTACTAATATGGCTCGTGTAGCTC | |
| | ATTCATTATATAAAGACGGAGACGGTTTTGGCGATCAGGAAAAAGG | |
| | TCCACGTACTCACATCTTATCTTTATTATTCCAACCATTAGTTAACGG | |
| | TACCGGTGAAAACTTATACTTTCAAGGTTCTGGTGGTGGTGGTTCTG | |
| | ACTACAAAGATGACGATGACAAAGGAACCGGTTAA | |
| 112 | ATGGTACCAAGTAGTAATGTATCAGCTATTCCTAATTCTTTTGAATT | Farnesene |
| | AATTCGTCGTTCAGCTCAATTTCAGGCTTCTGTATGGGGTGATTACTT | (C. sativus) |
| | TTTATCTTATCACTCTTTACCACCTGAGAAAGGTAATAAAGTAATGG | |
| | AAAAACAAACTGAAGAACTTAAAGAGGAAATCAAAATGGAATTAGT | |
| | TTCTACTACTAAAGATGAACCAGAGAAATTACGTTTAATTGACCTTA | |
| | TTCAACGTTTAGGTGTATGTTATCACTTTGAAAATGAAATTAACAAC | |
| | ATTTTACAACAATTACACCACATTACTATTACTTCTGAGAAAAACGG | |
| | TGACGATAATCCTTATAACATGACTTTATGTTTCCGTTTATTACGTCA | |
| | ACAAGGTTACAATGTATCTAGTGAACCTTTTGATCGTTTTCGTGGCA | |
| | AATGGGAATCTTCTTATGATAACAATGTAGAAGAACTTTTATCATTA | |
| | TATGAAGCATCTCAATTAAGAATGCAAGGTGAAGAAGCATTAGATG | |
| | AAGCATTCTGTTTTGCAACTGCACAATTAGAAGCTATTGTTCAAGAT | |
| | CCTACTACAGATCCAATGGTTGCAGCAGAAATCAGACAAGCATTAA | |
| | AATGGCCAATGTACAAAAACTTACCTCGTTTAAAAGCTCGTCATCAT | |
| | ATTGGTTTATATTCTGAGAAACCATGGCGTAATGAGTCATTACTTAA | |
| | TTTCGCAAAAATGGACTTCAATAAACTTCAAAATTTACATCAAACTG | |
| | AAATTGCATATATTTCTAAATGGTGGGACGATTACGGCTTTGCAGAA | |
| | AAACTTTCTTTCGCACGTAATCGTATTGTTGAAGGCTATTTCTTCGCA | |
| | TTAGGTATCTTTTTCGAACCTCAACTTTTAACAGCACGTCTTATAATG | |
| | ACAAAAGTAATCGCTATTGGTTCTATGTTAGATGACATTTATGATGT | |
| | TTATGGTACTTTTGAAGAGTTAAAACTTTTAACATTAGCTTTAGAAC | |
| | GTTGGGATAAATCAGAAACAAAACAATTACCTAATTACATGAAAAT | |
| | GTACTACGAAGCATTATTAGATGTTTTTGAAGAAATTGAGCAAGAA | |
| | ATGTCACAAAAAGAAACTGAAACAACACCATACTGTATTCATCACA | |
| | TGAAAGAAGCTACTAAAGAACTTGGACGTGTATTTTTAGTTGAAGCA | |
| | ACTTGGTGTAAAGAAGGTTATACTCCTAAAGTAGAGGAATACTTAG | |
| | ACATTGCTTTAATTTCTTTTGGTCATAAATTACTTATGGTAACTGCTT | |
| | TATTAGGTATGGGTTCTCACATGGCTACACAACAAATTGTACAATGG | |
| | ATTACATCTATGCCAAATATCTTAAAAGCATCTGCAGTAATATGTCG | |
| | TTTAATGAATGACATTGTATCTCATAAATTTGAACAAGAACGTGGTC | |
| | ATGTTGCTTCTGCTATCGAATGCTACATGGAACAAAACCACCTTAGT | |
| | GAATATGAAGCATTAATTGCTCTTCGTAAACAAATTGATGATTTATG | |
| | GAAAGACATGGTAGAAAATTACTGTGCAGTAATCACAGAAGACGAA | |
| | GTACCTCGTGGTGTTTTAATGCGTGTTTTAAATCTTACACGTTTATTC | |
| | AATGTTATTTACAAAGACGGTGATGGATACACACAAAGTCATGGTA | |
| | GTACAAAAGCTCACATTAAAAGTCTTTTAGTTGATAGTGTACCTCTT | |
| | GGTACCGGTGAAAATCTTTACTTTCAAGGTTCAGGTGGAGGTGGTTC | |
| | TGATTATAAAGATGATGATGACAAAGGAACCGGTTAA | |
| 113 | ATGGTACCAAAAGACATGAGTATTCCATTATTAGCAGCTGTATCTTC | Farnesene |
| | TAGTACAGAAGAAACAGTACGTCCTATCGCAGATTTTCATCCAACAC | (C. junos) |
| | TTTGGGGTAATCATTTTCTTAAATCTGCTGCTGACGTAGAAACTATT | |
| | GATGCAGCAACACAAGAGCAACACGCTGCATTAAAACAAGAAGTAC | |
| | GTCGTATGATTACTACAACAGCAAATAAACTTGCACAAAAACTTCAC | |
| | ATGATTGATGCTGTACAACGTTTAGGTGTTGCTTATCATTTTGAAAA | |
| | AGAAATTGAAGACGAATTAGGTAAAGTAAGTCACGATTTAGATTCA | |
| | GATGATTTATACGTTGTATCTTTACGTTTTCGTTTATTCCGTCAACAA | |
| | GGTGTAAAAATTAGTTGCGATGTTTTCGACAAATTCAAAGATGACGA | |
| | AGGAAAATTCAAAGAGTCTCTTATTAACGATATTAGAGGAATGTTAT | |
| | CATTATACGAAGCAGCTTACTTAGCTATTAGAGGTGAAGATATTTTA | |
| | GACGAAGCAATTGTTTTCACAACTACTCACTTAAAAAGTGTTATCTC | |
| | TATTAGTGATCATTCACATGCTAATAGTAATTTAGCTGAACAAATAC | |
| | GTCATAGTTTACAAATTCCACTTCGTAAAGCTGCTGCAAGATTAGAA | |
| | GCACGTTATTTCTTAGATATTTACTCTCGTGATGATTTACATGATGAA | |
| | ACATTACTTAAATTCGCTAAACTTGACTTTAACATTCTTCAAGCTGC | |
| | ACACCAAAAAGAAGCTAGTATTATGACTCGTTGGTGGAACGATTTA | |
| | GGTTTTCCTAAAAAAGTTCCTTATGCTCGTGACCGTATTATAGAAAC | |
| | TTATATTTGGATGTTATTAGGAGTTTCATACGAACCTAATTTAGCATT | |
| | TGGAAGAATTTTTGCAAGTAAAGTAGTATGTATGATTACAACAATTG | |
| | ATGATACATTTGATGCTTATGGTACATTTGAAGAGTTAACATTATTC | |
| | ACTGAAGCTGTTACACGTTGGGATATTGGTTTAATTGACACATTACC | |
| | TGAATATATGAAATTCATTGTAAAAGCTCTTTTAGACATTTACCGTG | |
| | AAGCTGAAGAAGAATTAGCTAAAGAAGGTAGATCATACGGTATTCC | |
| | ATACGCTAAACAAATGATGCAAGAGTTAATCATTTTATACTTTACTG | |
| | AGGCTAAATGGTTATACAAAGGTTACGTTCCTACATTTGACGAATAC | |
| | AAAAGTGTAGCTTTACGTTCTATTGGTCTTAGAACATTAGCAGTAGC | |
| | TTCATTTGTAGATTTAGGTGACTTTATTGCTACAAAAGACAATTTTG | |
| | AATGTATTCTTAAAAATGCAAAAAGTTTAAAAGCTACTGAAACAATT | |
| | GGCCGTTTAATGGATGATATAGCTGGTTACAAATTTGAACAGAAAC | |
| | GTGGTCATAACCCATCTGCTGTTGAGTGTTACAAAAATCAACACGGA | |
| | GTATCAGAAGAAGAAGCAGTTAAAGAGCTTTTATTAGAAGTTGCAA | |
| | ACAGTTGGAAAGATATTAACGAGGAACTTTTAAATCCAACTACAGTT | |
| | CCATTACCTATGTTACAGCGTTTATTATATTTTGCTCGTTCAGGTCAC | |
| | TTCATCTATGATGATGGACATGATCGTTATACACATTCTTTAATGAT | |
| | GAAAAGACAAGTTGCACTTTTATTAACTGAACCTTTAGCTATTGGTA | |
| | CCGGTGAAAACTTATACTTTCAAGGTTCAGGTGGTGGTGGATCTGAT | |
| | TATAAAGATGATGATGACAAAGGAACCGGTTAA | |
| 114 | ATGGTACCAGATTTAGCTGTTGAGATTGCAATGGACTTAGCTGTTGA | Farnesene |
| | TGACGTTGAGCGTCGTGTAGGTGACTATCATAGTAACCTTTGGGATG | (P. abies) |
| | ATGATTTTATTCAGAGTTTATCAACACCATACGGCGCATCATCATAT | |
| | CGTGAACGTGCTGAAAGATTAGTAGGAGAAGTTAAAGAAATGTTTA | |
| | CTTCTATTTCTATCGAAGATGGTGAACTTACATCTGATTTATTACAAC | |
| | GTTTATGGATGGTAGATAATGTAGAGCGTTTAGGCATTTCACGTCAT | |
| | TTCGAGAACGAAATAAAAGCAGCTATTGATTATGTTTATTCATATTG | |
| | GAGTGACAAAGGTATTGTACGTGGTCGTGATTCAGCTGTTCCTGACT | |
| | TAAATAGTATTGCTTTAGGTTTTCGTACATTACGTTTACACGGTTACA | |
| | CAGTTAGTAGTGATGTATTTAAAGTTTTCCAAGATCGTAAAGGTGAA | |
| | TTTGCTTGCAGTGCAATTCCAACTGAAGGAGATATTAAAGGAGTTTT | |
| | AAACTTACTTCGTGCAAGTTATATTGCATTCCCTGGTGAAAAAGTAA | |
| | TGGAAAAAGCTCAAACTTTTGCAGCAACATACCTTAAAGAAGCATT | |
| | ACAGAAAATTCAAGTAAGTAGTTTAAGTCGTGAAATCGAATATGTTC | |
| | TTGAATACGGTTGGTTAACTAACTTTCCTCGTTTAGAAGCACGTAAC | |
| | TATATTGACGTATTCGGTGAAGAAATTTGTCCATACTTCAAAAAACC | |
| | ATGTATTATGGTTGACAAACTTTTAGAATTAGCAAAATTAGAATTTA | |
| | ACTTATTTCACAGTCTTCAACAAACAGAGTTAAAACATGTTAGTCGT | |
| | TGGTGGAAAGATAGTGGTTTCTCTCAATTAACATTTACAAGACACCG | |
| | TCATGTTGAGTTTTATACATTAGCTAGTTGTATAGCAATTGAACCAA | |
| | AACACAGTGCTTTTCGTCTTGGTTTTGCTAAAGTTTGTTATTTAGGTA | |
| | TAGTTTTAGATGATATTTATGACACATTTGGTAAAATGAAAGAATTA | |
| | GAACTTTTTACTGCAGCAATCAAACGTTGGGACCCTTCTACTACAGA | |
| | ATGCTTACCTGAATACATGAAAGGTGTTTATATGGCTTTTTACAATT | |
| | GTGTTAATGAATTAGCACTTCAAGCAGAGAAAACACAAGGTCGTGA | |
| | TATGTTAAACTATGCACGTAAAGCATGGGAAGCTCTTTTTGATGCAT | |
| | TTTTAGAAGAAGCAAAATGGATCTCTTCTGGCTATTTACCAACATTC | |
| | GAAGAATACTTAGAAAATGGTAAAGTATCTTTTGGTTATCGTGCTGC | |
| | TACATTACAACCAATTTTAACATTAGATATTCCTTTACCTTTACATAT | |
| | TTTACAACAGATTGATTTTCCAAGTCGTTTTAATGATTTAGCTTCATC | |
| | TATTTTACGTTTAAGAGGTGATATCTGTGGTTACCAAGCTGAACGTA | |
| | GTCGTGGTGAAGAAGCATCATCAATTTCATGTTATATGAAAGATAAT | |
| | CCAGGTTCTACTGAAGAAGATGCATTATCTCACATTAATGCAATGAT | |
| | CTCAGACAATATTAACGAATTAAACTGGGAACTTTTAAAACCAAATT | |
| | CAAATGTACCAATTTCATCAAAAAAACATGCATTTGACATTCTTCGT | |
| | GCTTTCTATCACTTATACAAATATCGTGATGGCTTCTCTATCGCAAA | |
| | AATTGAAACTAAAAATCTTGTAATGCGTACAGTTTTAGAACCTGTAC | |
| | CAATGGGTACCGGTGAAAACTTATACTTTCAGGGTTCTGGTGGAGGT | |
| | GGTTCAGACTATAAAGATGATGATGATAAAGGAACCGGTTAA | |
| 115 | ATGGTACCAACAAGTGTATCAGTAGAATCAGGAACAGTATCTTGTTT | Bisabolene |
| | ATCATCAAACAACTTAATTAGACGTACAGCTAATCCACATCCTAACA | (P. abies) |
| | TTTGGGGATATGATTTTGTTCACTCACTTAAATCACCATATACACAC | |
| | GACTCATCATATCGTGAACGTGCTGAGACTTTAATTTCAGAAATAAA | |
| | AGTTATGCTTGGAGGTGGTGAATTAATGATGACTCCATCAGCTTATG | |
| | ATACAGCATGGGTAGCTCGTGTTCCATCAATTGACGGTAGTGCTTGT | |
| | CCACAATTTCCACAAACTGTTGAATGGATTCTTAAAAACCAATTAAA | |
| | AGATGGTAGTTGGGGAACTGAATCTCACTTCTTACTTAGTGACAGAT | |
| | TATTAGCTACATTAAGTTGTGTATTAGCATTATTAAAATGGAAAGTA | |
| | GCTGATGTTCAAGTAGAGCAAGGTATTGAGTTTATCAAACGTAATTT | |
| | ACAAGCTATTAAAGACGAACGTGATCAAGACAGTTTAGTAACTGAT | |
| | TTCGAGATTATTTTCCCATCACTTTTAAAAGAGGCTCAATCTTTAAAC | |
| | TTAGGCTTACCTTATGATTTACCATATATTAGATTATTACAAACAAA | |
| | ACGTCAAGAACGTCTTGCTAACTTAAGTATGGATAAAATTCACGGTG | |
| | GTACTTTATTATCATCTTTAGAGGGCATTCAAGATATAGTTGAATGG | |
| | GAAACAATTATGGATGTACAATCTCAAGATGGTTCTTTCTTATCATC | |
| | ACCAGCTTCTACAGCATGTGTATTCATGCATACAGGAGATATGAAAT | |
| | GTTTAGATTTCTTAAACAACGTATTAACTAAATTTGGTAGTAGTGTT | |
| | CCTTGTTTATACCCTGTAGATTTATTAGAACGTCTTTTAATTGTAGAT | |
| | AATGTAGAGCGTCTTGGTATTGACCGTCATTTTGAAAAAGAAATCAA | |
| | AGAGGCTTTAGATTATGTTTATCGTCATTGGAACGATCGTGGTATTG | |
| | GTTGGGGTCGTTTATCACCTATCGCAGACTTAGAAACAACAGCTTTA | |
| | GGTTTTCGTTTACTTCGTCTTCATCGTTACAATGTTTCTCCTGTAGTA | |
| | TTAGACAATTTCAAAGACGCAGATGGCGAGTTCTTCTGCAGTACAGG | |
| | TCAATTTAACAAAGATGTTGCAAGTATGTTATCTTTATACCGTGCTTC | |
| | TCAATTAGCTTTCCCTGAAGAATCAATTTTAGATGAAGCTAAATCAT | |
| | TCTCAACACAATATCTTCGTGAAGCATTAGAAAAATCAGAAACATTT | |
| | TCTTCTTGGAATCATCGTCAGAGTTTATCAGAAGAAATTAAATATGC | |
| | TTTAAAAACATCATGGCACGCTTCAGTTCCTCGTGTTGAAGCAAAAC | |
| | GTTATTGTCAGGTTTACCGTCAAGACTATGCTCATTTAGCAAAATCA | |
| | GTTTATAAACTTCCTAAAGTAAATAATGAGAAAATTCTTGAATTAGC | |
| | AAAATTAGATTTTAACATTATTCAATCTATCCATCAAAAAGAAATGA | |
| | AAAATGTTACATCATGGTTTCGTGATTCAGGCTTACCACTTTTCACAT | |
| | TTGCTCGTGAAAGACCTTTAGAGTTTTACTTTTTAATCGCTGGTGGA | |
| | ACATACGAACCTCAATACGCAAAATGTAGATTCTTATTTACAAAAGT | |
| | AGCTTGTTTACAAACTGTTTTAGACGATATGTACGATACTTACGGTA | |
| | CACCATCAGAGTTAAAATTATTTACTGAGGCAGTTCGTCGTTGGGAT | |
| | TTATCATTCACAGAAAACTTACCTGATTATATGAAATTATGCTACAA | |
| | AATTTACTATGATATTGTTCATGAAGTTGCTTGGGAAGTAGAAAAAG | |
| | AACAGGGACGTGAGCTTGTTTCATTTTTCCGTAAAGGTTGGGAAGAC | |
| | TATCTTTTAGGTTATTATGAAGAAGCTGAATGGTTAGCTGCTGAATA | |
| | CGTTCCTACTTTAGATGAATACATTAAAAACGGTATTACATCTATTG | |
| | GTCAACGTATTTTACTTTTATCAGGTGTACTTATTATGGAAGGTCAA | |
| | CTTTTATCACAAGAAGCTCTTGAAAAAGTAGATTATCCAGGTCGTCG | |
| | TGTTTTAACAGAATTAAACAGTTTAATTAGTCGTTTAGCAGACGATA | |
| | CTAAAACATACAAAGCAGAAAAAGCTCGTGGTGAACTTGCTAGTAG | |
| | TATTGAATGTTATATGAAAGACCACCCTGGTTGTCAAGAAGAAGAA | |
| | GCATTAAACCATATTTATGGCATTTTAGAACCAGCTGTTAAAGAATT | |
| | AACTCGTGAGTTTCTTAAAGCAGATCACGTACCATTCCCTTGCAAAA | |
| | AAATGTTATTTGATGAAACAAGAGTTACAATGGTAATTTTCAAAGAT | |
| | GGTGATGGTTTCGGTATTTCTAAATTAGAAGTAAAAGACCACATAAA | |
| | AGAATGTTTAATTGAGCCATTACCACTTGGTACCGGTGAAAATCTTT | |
| | ATTTTCAAGGTAGTGGTGGTGGCGGTTCTGACTACAAAGATGACGAC | |
| | GATAAAGGAACCGGTTAA | |
| 116 | ATGGTACCAGGTTCTGAAGTAAATAGACCTTTAGCAGACTTTCCAGC | Sesquiterpene |
| | AAACATTTGGGAAGACCCATTAACTTCTTTCTCAAAATCTGATCTTG | (A. thalania) |
| | GTACAGAAACATTTAAAGAGAAACATAGTACTTTAAAAGAAGCTGT | |
| | TAAAGAGGCATTTATGAGTTCTAAAGCTAATCCAATCGAAAATATCA | |
| | AATTCATAGATGCATTATGCCGTTTAGGAGTATCTTATCACTTTGAA | |
| | AAAGATATTGTAGAACAATTAGATAAATCATTTGATTGCTTAGATTT | |
| | TCCACAAATGGTACGTCAAGAAGGTTGCGATTTATATACAGTTGGTA | |
| | TTATCTTTCAAGTTTTTAGACAATTTGGTTTCAAATTAAGTGCTGATG | |
| | TTTTTGAAAAATTCAAAGATGAAAATGGTAAATTCAAAGGTCACTTA | |
| | GTAACTGATGCTTATGGTATGTTATCATTATACGAAGCTGCACAATG | |
| | GGGTACTCACGGTGAAGACATCATTGACGAAGCTCTTGCTTTTTCTC | |
| | GTAGTCACTTAGAAGAAATATCTAGTCGTAGTTCACCACACTTAGCA | |
| | ATTCGTATTAAAAACGCTTTAAAACATCCATATCATAAAGGTATTTC | |
| | ACGTATTGAAACACGTCAATACATTAGTTACTATGAAGAAGAAGAA | |
| | TCTTGTGATCCAACATTATTAGAGTTCGCTAAAATTGACTTTAACTTA | |
| | TTACAAATTTTACACCGTGAAGAGTTAGCTTGTGTAACTCGTTGGCA | |
| | TCATGAAATGGAATTTAAAAGTAAAGTAACTTACACACGTCATCGTA | |
| | TTACAGAAGCATATTTATGGAGTCTTGGAACATATTTTGAACCACAA | |
| | TACAGTCAAGCTCGTGTAATAACTACAATGGCATTAATCTTATTTAC | |
| | TGCTTTAGACGACATGTACGATGCTTACGGTACTATGGAGGAGTTAG | |
| | AGTTATTCACAGATGCTATGGACGAATGGTTACCAGTTGTTCCAGAT | |
| | GAAATTCCTATTCCAGATTCAATGAAATTCATTTACAATGTTACAGT | |
| | TGAATTTTACGATAAATTAGACGAAGAATTAGAAAAAGAAGGTCGT | |
| | TCTGGTTGTGGTTTCCATCTTAAAAAAAGTTTACAAAAAACAGCTAA | |
| | TGGATATATGCAAGAAGCAAAATGGCTTAAAAAAGATTACATTGCT | |
| | ACATTTGATGAGTATAAAGAAAATGCTATTTTATCTTCAGGTTATTA | |
| | TGCATTAATTGCAATGACATTTGTTCGTATGACTGATGTTGCTAAATT | |
| | AGATGCTTTTGAATGGTTAAGTAGTCACCCAAAAATTCGTGTAGCAA | |
| | GTGAAATCATTTCACGTTTTACAGACGATATTTCAAGTTATGAATTT | |
| | GAACACAAACGTGAACACGTTGCTACAGGTATTGATTGTTATATGCA | |
| | ACAATTCGGAGTTAGTAAAGAACGTGCTGTTGAAGTTATGGGCAAT | |
| | ATAGTTTCTGATGCATGGAAAGACTTAAATCAAGAACTTATGCGTCC | |
| | TCATGTTTTCCCATTTCCACTTCTTATGCGTGTTTTAAATCTTTCAAG | |
| | AGTAATTGATGTATTTTATCGTTACCAAGATGCATATACTAATCCAA | |
| | AATTACTTAAAGAGCACATTGTTTCTTTACTTATTGAAACTATTCCAA | |
| | TTGGTACCGGTGAAAACTTATACTTTCAAGGTAGTGGTGGAGGTGGT | |
| | TCTGATTATAAAGACGACGATGACAAAGGAACCGGTTAA | |
| 117 | ATGGTACCAGAGGCAATTAGAGTATTTGGCTTAAAACTTGGTTCAAA | Sesquiterpene |
| | ATTATCTATTCACTCACAAACAAATGCTTTTCCTGCATTCAAATTATC | (A. thalania) |
| | TCGTTTTCCATTAACATCTTTCCCTGGTAAACATGCTCACTTAGATCC | |
| | ATTAAAAGCAACAACTCATCCATTAGCTTTTGATGGTGAAGAAAATA | |
| | ACCGTGAGTTTAAAAACTTAGGTCCAAGTGAGTGGGGCCATCAATTT | |
| | CTTTCTGCTCATGTAGATTTATCTGAAATGGATGCATTAGAACGTGA | |
| | AATTGAAGCTCTTAAACCAAAAGTACGTGATATGTTAATATCAAGTG | |
| | AAAGTTCAAAAAAAAAAATCTTATTTCTTTATCTTTTAGTATCATTA | |
| | GGATTAGCTTATCACTTTGAAGATGAAATTAAAGAAAGTTTAGAGG | |
| | ATGGATTACAGAAAATTGAGGAAATGATGGCTTCAGAAGATGATCT | |
| | TCGTTTTAAAGGCGATAATGGTAAATTCAAAGAATGTTTAGCAAAA | |
| | GATGCTAAAGGTATTTTATCTCTTTATGAGGCTGCTCACATGGGTAC | |
| | AACAACTGATTATATTCTTGATGAGGCTTTATCATTTACTTTAACATA | |
| | TATGGAATCATTAGCAGCTTCAGGAACATGTAAAATCAACTTATCAC | |
| | GTCGTATTAGAAAAGCATTAGATCAACCTCAACACAAAAATATGGA | |
| | AATAATTGTAGCAATGAAATACATTCAATTTTATGAAGAAGAGGAA | |
| | GATTGCGATAAAACTTTACTTAAATTTGCTAAACTTAACTTTAAATT | |
| | CTTACAATTACACTATTTACAAGAACTTAAAATCTTATCTAAATGGT | |
| | ATAAAGACCAAGACTTTAAATCAAAATTACCTCCATATTTCCGTGAC | |
| | CGTCTTGTAGAATGTCATTTTGCATCATTAACATGTTTTGAGCCTAAA | |
| | TATGCTCGTGCACGTATTTTCTTATCTAAAATCTTCACTGTTCAAATT | |
| | TTCATTGACGATACTTGTGACCGTTACGCATCATTAGGTGAAGTTGA | |
| | GTCATTAGCTGACACTATCGAACGTTGGGACCCTGATGATCATGCTA | |
| | TGGACGGATTACCTGATTATCTTAAATCAGTAGTTAAATTTGTATTC | |
| | AATACATTTCAAGAATTTGAACGTAAATGTAAACGTTCACTTCGTAT | |
| | TAACTTACAAGTAGCAAAATGGGTTAAAGCTGGTCACTTACCATCTT | |
| | TTGATGAGTATCTTGATGTAGCTGGTTTAGAATTAGCTATTTCATTCA | |
| | CTTTCGCTGGTATCTTAATGGGCATGGAAAATGTTTGTAAACCTGAA | |
| | GCATACGAATGGTTAAAATCTCGTGACAAACTTGTTCGTGGTGTAAT | |
| | CACAAAAGTTCGTTTACTTAATGATATTTTTGGCTATGAAGATGATA | |
| | TGCGTCGTGGTTATGTAACAAATTCAATAAACTGCTACAAAAAACA | |
| | ATATGGAGTAACAGAGGAAGAAGCTATTCGTAAATTACATCAAATC | |
| | GTTGCTGATGGAGAGAAAATGATGAATGAAGAGTTCTTAAAACCTA | |
| | TTAATGTACCATATCAGGTTCCTAAAGTAGTTATTTTAGACACTTTAC | |
| | GTGCAGCTAATGTTTCATACGAAAAAGATGACGAATTTACACGTCCA | |
| | GGCGAACACCTTAAAAACTGCATTACATCTATTTACTTCGATTTAGG | |
| | TACCGGTGAAAACTTATACTTTCAAGGTAGTGGTGGCGGTGGTAGTG | |
| | ATTACAAAGATGATGATGATAAAGGAACCGGTTAA | |
| 118 | ATGGTACCAACTACAACATTATCATCTAACCTTAACTCACAATTCAT | GPP |
| | GCAGGTTTACGAGACTCTTAAATCAGAACTTATTCATGACCCATTAT | Chimera |
| | TTGAGTTCGATGACGATTCAAGACAATGGGTAGAACGTATGATTGAT | |
| | TATACTGTACCAGGTGGTAAAATGGTTCGTGGTTATAGTGTAGTAGA | |
| | TAGTTATCAATTACTTAAAGGTGAAGAACTTACAGAAGAAGAGGCA | |
| | TTTTTAGCTTGTGCACTTGGTTGGTGTACAGAATGGTTTCAAGCATTC | |
| | ATTCTTTTACATGATGATATGATGGATGGTAGTCACACAAGACGTGG | |
| | TCAACCATGTTGGTTTCGTTTACCTGAGGTTGGTGCTGTTGCTATTAA | |
| | TGATGGTGTTTTACTTCGTAATCACGTTCACCGTATTCTTAAAAAAC | |
| | ATTTTCAAGGTAAAGCATATTATGTTCATTTAGTTGATTTATTCAATG | |
| | AAACTGAATTTCAAACAATTAGTGGACAAATGATCGACTTAATTACA | |
| | ACATTAGTTGGTGAAAAAGACTTATCTAAATATTCATTAAGTATTCA | |
| | TCGTCGTATCGTTCAATACAAAACAGCATACTACTCATTTTACTTAC | |
| | CAGTTGCTTGTGCTTTACTTATGTTTGGTGAGGATCTTGATAAACATG | |
| | TAGAAGTTAAAAATGTTCTTGTTGAAATGGGTACATATTTTCAAGTT | |
| | CAAGATGATTATTTAGATTGTTTTGGTGCTCCAGAAGTTATTGGCAA | |
| | AATTGGTACTGATATTGAAGACTTTAAATGTTCATGGTTAGTAGTTA | |
| | AAGCATTAGAATTAGCAAATGAAGAACAGAAAAAAACTTTACACGA | |
| | AAATTATGGAAAAAAAGATCCAGCATCAGTTGCTAAAGTTAAAGAA | |
| | GTATACCACACACTTAATTTACAAGCTGTTTTCGAAGATTATGAAGC | |
| | AACATCATACAAAAAACTTATTACTTCTATTGAAAATCACCCATCTA | |
| | AAGCTGTTCAAGCTGTTTTAAAATCTTTCTTAGGCAAAATATACAAA | |
| | CGTCAAAAAGGTACCGGTGAAAACTTATACTTTCAAGGTTCTGGTGG | |
| | CGGTGGAAGTGATTACAAAGATGATGACGATAAAGGAACCGGTTAA | |
| 119 | ATGGTACCAAGTCAACCTTACTGGGCTGCAATTGAAGCAGACATTG | GPPS- |
| | AAAGATATTTAAAAAAATCAATTACAATTCGTCCACCAGAAACTGT | LSU + SSU |
| | ATTTGGTCCTATGCACCATTTAACATTTGCTGCTCCTGCTACTGCAGC | fusion |
| | TAGTACATTATGCCTTGCTGCTTGTGAATTAGTTGGCGGTGATCGTA | |
| | GTCAAGCTATGGCAGCTGCTGCTGCTATCCATTTAGTTCATGCAGCT | |
| | GCTTACGTTCACGAACATCTTCCTTTAACAGATGGATCACGTCCTGT | |
| | AAGTAAACCTGCTATTCAACATAAATATGGTCCAAACGTTGAACTTT | |
| | TAACAGGTGATGGTATCGTTCCTTTCGGTTTTGAGTTATTAGCAGGTT | |
| | CAGTAGATCCAGCACGTACTGATGACCCTGATCGTATTTTACGTGTA | |
| | ATTATTGAAATTTCTCGTGCTGGTGGACCAGAAGGCATGATTTCTGG | |
| | TTTACACCGTGAGGAAGAAATCGTAGATGGTAACACATCATTAGAC | |
| | TTTATAGAATATGTATGCAAAAAAAAATACGGTGAAATGCACGCAT | |
| | GTGGTGCAGCTTGCGGAGCTATTTTAGGTGGAGCTGCTGAAGAAGA | |
| | AATTCAAAAACTTCGTAACTTTGGTCTTTATCAAGGCACATTACGTG | |
| | GTATGATGGAAATGAAAAATAGTCATCAGTTAATTGACGAAAATAT | |
| | CATTGGAAAACTTAAAGAACTTGCTCTTGAAGAATTAGGTGGATTCC | |
| | ACGGTAAAAACGCTGAATTAATGAGTTCTTTAGTTGCTGAACCTAGT | |
| | TTATATGCAGCTTCATCAAATAACTTAGGTATCGAAGGTCGTTTTGA | |
| | CTTTGACGGTTACATGCTTCGTAAAGCAAAATCTGTAAATAAAGCAT | |
| | TAGAAGCTGCTGTTCAAATGAAAGAACCACTTAAAATTCACGAATC | |
| | AATGCGTTATTCATTATTAGCTGGTGGTAAACGTGTTCGTCCAATGT | |
| | TATGTATTGCAGCTTGTGAACTTGTTGGTGGTGACGAATCTACAGCA | |
| | ATGCCTGCAGCATGTGCTGTTGAAATGATTCACACAATGTCTTTAAT | |
| | GCATGATGACCTTCCATGTATGGATAACGATGACTTACGTCGTGGTA | |
| | AACCTACAAACCACATGGCTTTTGGTGAGTCTGTAGCTGTTCTTGCT | |
| | GGTGATGCATTACTTAGTTTTGCTTTTGAACATGTTGCTGCTGCAACA | |
| | AAAGGCGCACCACCTGAACGTATCGTACGTGTATTAGGTGAATTAG | |
| | CTGTTAGTATTGGTTCAGAAGGACTTGTAGCAGGTCAAGTTGTAGAC | |
| | GTTTGTTCTGAAGGCATGGCTGAAGTAGGATTAGATCATCTTGAATT | |
| | TATTCACCATCATAAAACTGCTGCATTATTACAAGGTTCAGTTGTTTT | |
| | AGGTGCAATATTAGGAGGCGGTAAAGAAGAAGAAGTAGCTAAACTT | |
| | CGTAAATTTGCTAACTGTATTGGTTTACTTTTCCAAGTTGTTGATGAT | |
| | ATTTTAGATGTTACTAAAAGTAGTAAAGAGTTAGGTAAAACTGCAG | |
| | GTAAAGACTTAGTAGCTGATAAAACTACATATCCTAAACTTATAGGC | |
| | GTTGAAAAATCAAAAGAATTTGCTGACCGTTTAAATCGTGAAGCAC | |
| | AAGAACAATTATTACATTTTCATCCTCACCGTGCTGCTCCATTAATC | |
| | GCTTTAGCTAACTACATCGCTTACCGTGATAATGGTACCGGTGAAAA | |
| | CTTATACTTCCAGGGTAGTGGTGGTGGCGGATCAGATTATAAAGATG | |
| | ACGATGATAAAGGAACCGGTTAA | |
| 120 | ATGGTACCAGTAACAGCAGCACGTGCAACACCAAAATTAAGTAATA | Geranyl- |
| | GAAAATTACGTGTTGCTGTAATTGGAGGCGGTCCAGCAGGAGGTGC | geranyl |
| | AGCTGCTGAAACATTAGCACAAGGAGGTATTGAAACAATTCTTATC | reductase |
| | GAACGTAAAATGGATAATTGTAAACCATGTGGTGGTGCTATTCCATT | (A. thalania) |
| | ATGTATGGTAGGAGAGTTCAATTTACCTTTAGACATTATTGACCGTC | |
| | GTGTAACAAAAATGAAAATGATCTCTCCTTCAAACATTGCAGTTGAT | |
| | ATCGGTCGTACACTTAAAGAACACGAATATATTGGTATGGTTCGTCG | |
| | TGAGGTACTTGATGCTTATCTTCGTGAACGTGCAGAAAAATCAGGTG | |
| | CTACTGTTATTAACGGTTTATTCTTAAAAATGGATCACCCAGAAAAT | |
| | TGGGATTCACCATATACACTTCACTACACAGAGTATGATGGAAAAA | |
| | CAGGTGCTACAGGAACTAAAAAAACTATGGAAGTAGATGCTGTTAT | |
| | TGGTGCTGATGGTGCTAATTCTCGTGTTGCAAAAAGTATTGACGCAG | |
| | GTGATTATGATTATGCTATTGCATTTCAAGAACGTATTCGTATACCT | |
| | GATGAGAAAATGACTTATTATGAGGACTTAGCTGAGATGTATGTAG | |
| | GTGATGATGTATCACCAGACTTCTACGGTTGGGTATTCCCAAAATGT | |
| | GATCATGTAGCTGTTGGTACAGGTACTGTAACACATAAAGGTGATAT | |
| | CAAAAAATTCCAGTTAGCTACACGTAATCGTGCTAAAGATAAAATTC | |
| | TTGGTGGCAAAATAATCCGTGTAGAGGCTCATCCTATTCCAGAGCAT | |
| | CCTAGACCACGTCGTTTATCAAAACGTGTTGCATTAGTAGGCGACGC | |
| | AGCAGGTTACGTTACTAAATGTTCAGGAGAAGGAATTTACTTCGCAG | |
| | CTAAATCTGGTCGTATGTGTGCTGAAGCTATCGTTGAAGGTTCACAA | |
| | AATGGCAAAAAAATGATAGATGAAGGCGATTTAAGAAAATACTTAG | |
| | AAAAATGGGATAAAACTTACTTACCAACTTATCGTGTTTTAGATGTA | |
| | CTTCAAAAAGTTTTCTATCGTTCTAACCCAGCTCGTGAGGCTTTTGTT | |
| | GAAATGTGTAACGATGAGTATGTACAGAAAATGACATTTGATTCTTA | |
| | CCTTTATAAACGTGTAGCTCCTGGTAGTCCATTAGAAGATATCAAAT | |
| | TAGCTGTAAATACTATTGGTTCACTTGTTCGTGCTAACGCATTACGTC | |
| | GTGAAATTGAGAAATTATCAGTAGGTACCGGTGAGAATCTTTACTTT | |
| | CAAGGATCAGGTGGTGGTGGTTCTGATTATAAAGATGACGATGATA | |
| | AAGGAACCGGTTAA | |
| 121 | ATGGTACCAGTAGCTGTTATTGGTGGTGGTCCAAGTGGCGCTTGTGC | Geranyl- |
| | AGCAGAAACTTTAGCAAAAGGTGGTGTAGAAACTTTCTTACTTGAGC | geranyl |
| | GTAAATTAGATAATTGTAAACCTTGTGGAGGTGCAATTCCATTATGT | reductase |
| | ATGGTTGAAGAATTTGATTTACCAATGGAAATAATTGACCGTCGTGT | (C. reinhardtii) |
| | TACTAAAATGAAAATGATATCACCTTCAAACCGTGAAGTTGATGTTG | |
| | GAAAAACTTTATCAGAAACTGAATGGATCGGTATGTGTCGTCGTGA | |
| | AGTATTTGACGATTACTTAAGAAACCGTGCACAGAAATTAGGTGCTA | |
| | ATATTGTTAACGGTTTATTCATGCGTTCAGAACAACAATCTGCAGAG | |
| | GGTCCATTCACAATTCACTATAATTCTTATGAAGACGGTAGTAAAAT | |
| | GGGAAAACCTGCTACTTTAGAAGTTGATATGATAATTGGTGCAGATG | |
| | GAGCAAATTCTCGTATTGCAAAAGAGATAGATGCAGGTGAATACGA | |
| | CTACGCTATAGCTTTTCAAGAACGTATTCGTATTCCTGATGATAAAA | |
| | TGAAATATTACGAAAACCTTGCTGAAATGTATGTAGGTGATGACGTA | |
| | TCTCCTGATTTCTATGGTTGGGTTTTTCCTAAATATGATCACGTTGCT | |
| | GTTGGTACAGGTACTGTTGTAAACAAAACAGCTATTAAACAATATCA | |
| | ACAGGCAACACGTGACAGATCAAAAGTTAAAACAGAAGGTGGCAA | |
| | AATTATACGTGTTGAAGCACACCCAATTCCAGAACATCCACGTCCAC | |
| | GTCGTTGTAAAGGTCGTGTTGCATTAGTAGGCGACGCAGCTGGTTAT | |
| | GTTACAAAATGTTCTGGCGAGGGCATTTACTTTGCTGCTAAATCTGG | |
| | TAGAATGGCTGCTGAAGCTATTGTAGAAGGTTCTGCTAACGGTACAA | |
| | AAATGTGTGGTGAGGATGCAATTCGTGTTTATTTAGATAAATGGGAT | |
| | CGTAAATATTGGACAACATACAAAGTATTAGACATTTTACAAAAAG | |
| | TATTTTATCGTAGTAATCCAGCACGTGAAGCATTTGTTGAATTATGT | |
| | GAAGATAGTTATGTACAGAAAATGACATTTGATTCATACTTATATAA | |
| | AACTGTTGTTCCAGGAAACCCATTAGACGACGTAAAATTACTTGTTC | |
| | GTACAGTATCTTCTATTTTACGTTCAAATGCTTTACGTTCTGTTAATT | |
| | CTAAATCTGTAAATGTTTCTTTCGGCTCTAAAGCAAATGAGGAACGT | |
| | GTTATGGCTGCAGGTACCGGTGAAAATCTTTATTTTCAAGGTTCAGG | |
| | AGGTGGTGGTTCAGATTATAAAGATGATGATGACAAAGGAACCGGT | |
| | TAA | |
| 122 | ATGGTACCAGCAATGGCAGTACCATTAGATGTAGTAATTACATATCC | Chlorophyllido |
| | TTCTTCAGGTGCTGCTGCTTATCCAGTACTTGTTATGTATAACGGTTT | hydrolase |
| | CCAAGCTAAAGCTCCATGGTATCGTGGTATTGTAGATCATGTTTCTA | (C. reinhardtii) |
| | GTTGGGGTTACACAGTTGTTCAATATACAAATGGTGGCTTATTTCCT | |
| | ATTGTTGTAGATCGTGTTGAGTTAACTTATTTAGAGCCATTATTAACT | |
| | TGGTTAGAAACACAAAGTGCTGATGCTAAATCTCCTTTATACGGTCG | |
| | TGCAGATGTTTCTCGTTTAGGTACAATGGGTCATTCACGTGGTGGTA | |
| | AATTAGCAGCTTTACAATTTGCTGGACGTACAGATGTAAGTGGTTGT | |
| | GTATTATTTGACCCTGTAGATGGAAGTCCAATGACACCAGAATCTGC | |
| | TGATTATCCTTCAGCTACAAAAGCATTAGCAGCAGCTGGTCGTTCTG | |
| | CTGGCTTAGTAGGTGCAGCTATTACAGGTTCATGTAATCCAGTAGGT | |
| | CAAAATTACCCAAAATTCTGGGGTGCTTTAGCTCCTGGTTCTTGGCA | |
| | AATGGTATTATCACAAGCTGGTCACATGCAATTTGCTCGTACTGGTA | |
| | ATCCATTCTTAGATTGGTCATTAGACCGTTTATGTGGTCGTGGTACA | |
| | ATGATGAGTTCAGATGTTATTACATATAGTGCAGCATTTACTGTTGC | |
| | TTGGTTTGAAGGTATTTTTCGTCCTGCTCAAAGTCAAATGGGTATTTC | |
| | TAATTTCAAAACTTGGGCTAATACTCAAGTTGCAGCTCGTAGTATCA | |
| | CTTTTGATATTAAACCTATGCAATCTCCTCAGGGTACCGGTGAAAAC | |
| | CTTTACTTTCAAGGTAGTGGTGGTGGAGGAAGTGATTATAAAGATGA | |
| | TGATGACAAAGGAACCGGTTAA | |
| 123 | ATGGTACCAGCACCACCAAAACCAGTTCGTATAACTTGTCCAACAGT | Chlorophyllido |
| | AGCTGGCACTTATCCTGTTGTTTTATTCTTTCACGGTTTTTATCTTCGT | hydrolase |
| | AACTATTTCTATTCAGATGTTTTAAATCATATTGCTAGTCATGGTTAC | (A. thalania) |
| | ATCTTAGTTGCACCACAATTATGTAAACTTTTACCTCCAGGTGGCCA | |
| | AGTAGAAGTTGATGACGCTGGTTCAGTTATTAACTGGGCTTCAGAGA | |
| | ATCTTAAAGCACACCTTCCAACTTCTGTTAATGCTAATGGTAAATAT | |
| | ACATCTTTAGTTGGACATTCACGTGGTGGCAAAACAGCTTTCGCAGT | |
| | TGCATTAGGTCACGCAGCTACATTAGATCCATCAATTACATTTTCAG | |
| | CATTAATTGGTATTGATCCAGTAGCAGGAACTAACAAATACATTCGT | |
| | ACAGATCCACACATCTTAACTTATAAACCTGAATCATTTGAATTAGA | |
| | TATTCCTGTAGCTGTTGTAGGCACTGGTCTTGGTCCAAAATGGAATA | |
| | ACGTAATGCCTCCATGCGCACCTACAGATTTAAACCACGAAGAATTT | |
| | TACAAAGAATGTAAAGCTACTAAAGCTCACTTTGTTGCTGCTGATTA | |
| | TGGTCACATGGACATGTTAGACGACGATCTTCCAGGTTTTGTAGGCT | |
| | TCATGGCTGGTTGTATGTGTAAAAATGGTCAACGTAAAAAATCAGA | |
| | AATGCGTTCTTTTGTAGGTGGTATAGTTGTAGCATTCTTAAAATATTC | |
| | TTTATGGGGTGAAAAAGCTGAAATAAGATTAATTGTTAAAGATCCTA | |
| | GTGTATCTCCTGCTAAATTAGACCCATCACCAGAATTAGAAGAAGCA | |
| | TCAGGTATTTTTGTTGGTACCGGTGAAAATCTTTATTTTCAAGGTTCA | |
| | GGTGGAGGTGGTTCTGATTATAAAGATGATGATGACAAAGGAACCG | |
| | GTTAA | |
| 124 | ATGGTACCAGCTACACCAGTTGAAGAAGGTGATTATCCAGTTGTAAT | Chlorophyllido |
| | GTTATTACATGGCTACCTTTTATATAATTCATTTTATTCACAATTAAT | hydrolase |
| | GTTACATGTATCATCTCACGGTTTCATCTTAATTGCTCCACAATTATA | (A. thalania) |
| | CTCAATTGCTGGTCCTGATACTATGGATGAAATTAAAAGTACTGCTG | |
| | AGATTATGGACTGGTTATCAGTTGGTTTAAATCACTTTTTACCAGCTC | |
| | AAGTTACACCTAATTTATCTAAATTTGCATTATCTGGTCATAGTCGTG | |
| | GTGGTAAAACTGCTTTTGCTGTAGCATTAAAAAAATTTGGTTATTCT | |
| | TCAAACTTAAAAATTAGTACTTTAATTGGTATTGATCCAGTAGACGG | |
| | AACAGGTAAAGGTAAACAAACTCCACCTCCTGTTTTAGCATATTTAC | |
| | CTAATAGTTTTGACTTAGACAAAACACCAATTTTAGTAATTGGTTCA | |
| | GGTTTAGGTGAAACTGCACGTAATCCTTTATTTCCTCCATGTGCTCCT | |
| | CCAGGTGTTAACCACCGTGAGTTTTTCCGTGAATGTCAAGGTCCAGC | |
| | ATGGCACTTTGTTGCTAAAGATTATGGTCATTTAGACATGCTTGATG | |
| | ATGATACAAAAGGTATTCGTGGCAAATCTAGTTACTGTTTATGCAAA | |
| | AATGGTGAAGAACGTCGTCCAATGCGTCGTTTCGTTGGTGGTTTAGT | |
| | TGTTAGTTTTCTTAAAGCATATCTTGAAGGTGATGATCGTGAATTAG | |
| | TAAAAATCAAAGATGGTTGTCATGAAGATGTACCTGTTGAAATTCAA | |
| | GAATTTGAAGTAATTATGGGTACCGGTGAAAATCTTTACTTTCAAGG | |
| | TTCAGGCGGTGGAGGTTCAGATTATAAAGATGATGATGACAAAGGA | |
| | ACCGGTTAA | |
| 125 | ATGGTACCAAGTCACAAAAAAAAAAACGTAATCTTCTTCGTAACTG | Phosphatase |
| | ATGGTATGGGTCCTGCTTCTCTTTCAATGGCTCGTTCATTTAATCAAC | (S. cerevisiae) |
| | ACGTTAATGATTTACCAATTGATGATATTTTAACATTAGATGAACAT | |
| | TTTATTGGAAGTTCAAGAACACGTTCATCAGATTCACTTGTAACTGA | |
| | CTCAGCTGCTGGAGCTACAGCTTTTGCTTGTGCACTTAAATCATACA | |
| | ATGGTGCTATAGGTGTAGATCCACACCATCGTCCATGTGGAACTGTT | |
| | TTAGAAGCTGCTAAATTAGCAGGTTATTTAACAGGATTAGTAGTTAC | |
| | TACACGTATTACTGATGCTACACCAGCTAGTTTCTCAAGTCACGTAG | |
| | ATTATCGTTGGCAAGAAGATTTAATTGCAACACACCAATTAGGTGAA | |
| | TATCCTTTAGGACGTGTTGTTGATCTTCTTATGGGTGGTGGTCGTTCT | |
| | CACTTTTATCCTCAAGGTGAAAAAGCTAGTCCATACGGTCACCACGG | |
| | TGCACGTAAAGATGGTCGTGATTTAATCGATGAAGCTCAAAGTAAT | |
| | GGCTGGCAGTATGTAGGAGATCGTAAAAATTTTGATTCTTTACTTAA | |
| | ATCACATGGTGAAAATGTTACTTTACCATTTTTAGGTTTATTTGCTGA | |
| | CAACGATATCCCATTTGAAATTGATCGTGATGAAAAAGAATATCCTA | |
| | GTTTAAAAGAACAAGTAAAAGTAGCATTAGGTGCTTTAGAAAAAGC | |
| | AAGTAACGAAGATAAAGATAGTAATGGTTTCTTTTTAATGGTAGAA | |
| | GGTTCTCGTATTGATCATGCTGGCCATCAAAACGATCCTGCATCTCA | |
| | AGTACGTGAAGTATTAGCATTTGATGAGGCTTTTCAATATGTATTAG | |
| | AATTTGCAGAAAACAGTGATACAGAAACAGTATTAGTAAGTACATC | |
| | AGATCATGAAACAGGTGGTTTAGTTACTTCAAGACAAGTAACAGCA | |
| | TCATACCCACAATATGTATGGTATCCTCAAGTATTAGCTAACGCTAC | |
| | ACATAGTGGAGAGTTTCTTAAACGTAAATTAGTTGATTTCGTTCATG | |
| | AACACAAAGGCGCATCATCAAAAATAGAAAACTTCATAAAACACGA | |
| | AATTCTTGAAAAAGATTTAGGTATTTATGATTATACAGATTCTGACT | |
| | TAGAAACACTTATTCATTTAGATGATAACGCTAATGCAATTCAAGAT | |
| | AAACTTAATGATATGGTAAGTTTTAGAGCTCAAATTGGTTGGACAAC | |
| | ACATGGTCATTCAGCAGTTGATGTAAACATATATGCTTACGCAAACA | |
| | AAAAAGCTACATGGTCTTATGTTCTTAATAACTTACAAGGTAATCAC | |
| | GAAAACACAGAAGTTGGTCAATTCTTAGAGAATTTCTTAGAATTAAA | |
| | CTTAAATGAAGTTACTGATTTAATCCGTGATACAAAACATACTTCTG | |
| | ATTTTGACGCAACAGAAATAGCAAGTGAGGTTCAACACTATGATGA | |
| | ATATTACCACGAATTAACAAATGGTACCGGTGAAAATCTTTATTTTC | |
| | AAGGTTCTGGTGGAGGTGGCAGTGATTATAAAGATGATGATGACAA | |
| | AGGAACCGGTTAA | |
| 126 | ATGGTACCACACAAGTTCACAGGTGTTAACGCTAAATTCCAGCAACC | FPP A118W |
| | AGCATTAAGAAATTTATCTCCAGTGGTAGTTGAGCGCGAACGTGAG | (G. gallus) |
| | GAATTTGTAGGATTCTTTCCACAAATTGTTCGTGACTTAACTGAAGA | |
| | TGGTATTGGTCATCCAGAAGTAGGTGACGCTGTAGCTCGTCTTAAAG | |
| | AAGTATTACAATACAACGCACCTGGTGGTAAATGCAATAGAGGTTT | |
| | AACAGTTGTTGCAGCTTACCGTGAACTTTCTGGACCAGGTCAAAAAG | |
| | ACGCTGAAAGTCTTCGTTGTGCTTTAGCAGTAGGATGGTGTATTGAA | |
| | TTATTCCAAGCCTTTTTCTTAGTTTGGGACGATATAATGGACCAGTC | |
| | ATTAACTAGACGTGGTCAATTATGTTGGTACAAGAAAGAAGGTGTT | |
| | GGTTTAGATGCAATAAATGATTCTTTTCTTTTAGAAAGCTCTGTGTAT | |
| | CGCGTTCTTAAAAAGTATTGCCGTCAACGTCCATATTATGTACATTT | |
| | ATTAGAGCTTTTTCTTCAAACAGCTTACCAAACAGAATTAGGACAAA | |
| | TGTTAGATTTAATCACTGCTCCTGTATCTAAGGTAGATTTAAGCCATT | |
| | TCTCAGAAGAACGTTACAAAGCTATTGTTAAGTATAAAACTGCTTTC | |
| | TATTCATTCTATTTACCAGTTGCAGCAGCTATGTATATGGTTGGTATA | |
| | GATTCTAAAGAAGAACATGAAAACGCAAAAGCTATTTTACTTGAGA | |
| | TGGGTGAATACTTCCAAATTCAAGATGATTATTTAGATTGTTTTGGC | |
| | GATCCTGCTTTAACAGGTAAAGTAGGTACTGATATTCAAGATAACAA | |
| | ATGTTCATGGTTAGTTGTGCAATGCTTACAAAGAGTAACACCAGAAC | |
| | AACGTCAACTTTTAGAAGATAATTACGGTCGTAAAGAACCAGAAAA | |
| | AGTTGCTAAAGTTAAAGAATTATATGAGGCTGTAGGTATGAGAGCC | |
| | GCCTTTCAACAATACGAAGAAAGTAGTTACCGTCGTCTTCAAGAGTT | |
| | AATTGAGAAACATTCTAATCGTTTACCAAAAGAAATTTTCTTAGGTT | |
| | TAGCTCAGAAAATATACAAACGTCAAAAAGGTACCGGTGAAAACTT | |
| | ATACTTTCAAGGCTCAGGTGGCGGTGGAAGTGATTACAAAGATGAT | |
| | GATGATAAAGGAACCGGTTAA | |

TABLE 6

Nucleic acids encoding exemplary isoprenoid producing
enzymes (with restriction enzyme sites

| | | Enzyme |
|---|---|---|
| SEQ | Codon-biased, Synthesized Gene Sequence | encoded |
| ID NO | w/ Cloning Sites | (synthetic) |
| 127 | CATATGGTACCAAGACGTTCAGGTAACTATAATCCTAGCCGTTGGGA | Limonene |
| | CGTAAATTTCATTCAATCTTTATTATCTGATTATAAAGAAGATAAAC | (M. spicata) |
| | ACGTTATTAGAGCTTCTGAATTAGTAACACTTGTTAAGATGGAATTA | |
| | GAAAAAGAAACAGATCAAATCCGTCAATTAGAATTAATTGACGATT | |
| | TACAACGTATGGGTTTATCTGATCATTTCCAAAACGAATTTAAAGAA | |
| | ATCTTATCAAGTATTTACTTAGATCATCATTATTACAAAAATCCATTT | |
| | CCAAAAGAAGAGCGTGATTTATACTCAACTAGCTTAGCTTTTCGTTT | |
| | ATTACGTGAACACGGTTTTCAAGTAGCACAAGAAGTTTTTGATTCAT | |
| | TCAAAAATGAAGAGGGTGAATTTAAGGAGAGCTTATCTGACGATAC | |
| | TCGTGGCTTATTACAATTATATGAAGCATCATTCTTATTAACAGAGG | |
| | GTGAAACAACCTTAGAAAGTGCACGCGAATTTGCTACAAAATTTTTA | |
| | GAAGAAAAAGTTAACGAAGGTGGCGTTGATGGTGACTTATTAACAA | |
| | GAATTGCTTACTCATTAGATATTCCCTTACATTGGCGCATTAAACGT | |
| | CCTAATGCCCCAGTTTGGATTGAATGGTATCGTAAACGTCCAGATAT | |
| | GAACCCAGTGGTTTTAGAATTAGCAATTTTAGACTTAAACATTGTAC | |
| | AAGCTCAATTTCAAGAGGAATTAAAAGAGTCTTTTCGCTGGTGGCGT | |
| | AATACTGGTTTTGTTGAGAAATTACCATTTGCACGTGATCGTTTAGTT | |
| | GAATGTTACTTTTGGAACACTGGTATTATTGAACCACGTCAACACGC | |
| | ATCAGCTCGTATTATGATGGGTAAAGTAAATGCATTAATTACAGTAA | |
| | TTGATGACATCTATGATGTTTATGGAACACTTGAAGAATTAGAACAA | |
| | TTCACTGATTTAATTCGCAGATGGGACATAAACTCAATAGATCAATT | |
| | ACCAGATTATATGCAATTATGTTTTCTTGCATTAAACAATTTCGTTGA | |
| | TGACACTTCATACGATGTTATGAAAGAAAAGGGTGTTAATGTTATTC | |
| | CTTACTTACGTCAATCTTGGGTAGACCTTGCAGACAAATATATGGTA | |
| | GAAGCACGTTGGTTCTACGGTGGCCATAAACCATCATTAGAAGAAT | |
| | ACTTAGAAAATTCTTGGCAATCTATCTCAGGTCCATGTATGTTAACT | |
| | CATATATTCTTTCGTGTAACAGATAGCTTTACTAAAGAAACTGTTGA | |
| | TTCTCTTTACAAATATCATGATTTAGTTAGATGGTCATCATTCGTGCT | |
| | TCGTCTTGCTGACGACTTAGGTACAAGCGTTGAAGAAGTATCTCGTG | |
| | GTGATGTGCCAAAATCTTTACAATGCTACATGAGTGATTATAACGCT | |
| | AGTGAGGCTGAAGCACGTAAACACGTAAAATGGTTAATTGCAGAAG | |
| | TATGGAAAAAGATGAATGCAGAACGTGTTTCTAAAGATAGTCCTTTT | |
| | GGTAAAGATTTTATAGGTTGTGCTGTTGATTTAGGTCGTATGGCTCA | |
| | ATTAATGTATCACAATGGAGATGGTCATGGTACTCAACACCCTATTA | |
| | TTCATCAACAAATGACACGTACTTTATTTGAACCATTCGCTGGTACC | |
| | GGTGAAAACTTATACTTTCAAGGCTCAGGTGGCGGTGGAAGTGATT | |
| | ACAAAGATGATGATGATAAAGGAACCGGTTAATCTAGACTCGAG | |
| 128 | CATATGGTACCAAGACGTACTGGTGGCTATCAACCTACACTTTGGGA | Cineole (S. officinalis) |
| | TTTTTCAACAATTCAATTATTTGATAGTGAATATAAAGAAGAAAAAC | |
| | ATCTTATGCGTGCTGCTGGTATGATTGCTCAAGTGAACATGTTACTT | |
| | CAAGAAGAAGTAGACAGCATCCAACGTCTTGAATTAATTGATGACT | |
| | TACGTCGTTTAGGTATATCTTGCCACTTTGATCGTGAAATTGTAGAG | |
| | ATTTTAAACAGTAAATACTACACCAACAATGAAATTGATGAATCAG | |
| | ATTTATACAGTACAGCACTTAGATTCAAACTTTTACGTCAATATGAT | |
| | TTTAGCGTTAGCCAAGAAGTTTTTGATTGTTTTAAAAATGACAAAGG | |
| | TACAGATTTCAAACCATCATTAGTTGACGATACACGTGGCTTATTAC | |
| | AATTATATGAAGCATCATTTTTATCAGCTCAGGGTGAAGAAACTTTA | |
| | CATTTAGCACGTGATTTTGCTACTAAATTCTTACATAAAAGAGTTTT | |
| | AGTAGATAAAGATATCAATTTATTATCTAGTATCGAGCGTGCTTTAG | |
| | AATTACCAACACACTGGCGTGTACAAATGCCTAACGCTAGATCATTC | |
| | ATCGACGCATATAAAAGAAGACCAGACATGAACCCTACAGTATTAG | |
| | AGTTAGCAAAACTTGACTTTAACATGGTTCAAGCACAGTTCCAACAA | |
| | GAATTAAAAGAAGCCAGTCGCTGGTGGAACTCTACAGGATTAGTAC | |
| | ATGAATTACCATTTGTACGTGATCGTATTGTGGAATGTTATTATTGG | |
| | ACTACTGGTGTAGTAGAACGTCGTGAACACGGTTACGAACGTATTAT | |
| | GTTAACAAAAATTAACGCTTTAGTTACAACAATCGATGATGTTTTTG | |
| | ACATTTATGGTACTTTAGAAGAATTACAACTTTTTACAACTGCTATTC | |
| | AAAGATGGGACATTGAGTCTATGAAACAACTTCCACCCTATATGCA | |
| | AATCTGCTACTTAGCTTTATTCAACTTCGTAAATGAGATGGCTTACG | |
| | ATACATTACGTGATAAAGGTTTTAATAGTACTCCATATTTACGCAAA | |
| | GCCTGGGTAGACTTAGTAGAAAGCTACTTAATTGAAGCTAAATGGT | |
| | ATTATATGGGTCACAAACCAAGTTTAGAAGAGTACATGAAAAACTC | |
| | ATGGATTTCTATCGGAGGTATTCCAATTTTATCACATTTATTCTTTCG | |
| | TTTAACAGACAGTATCGAAGAAGAAGACGCTGAATCAATGCATAAA | |
| | TATCACGATATAGTACGTGCCTCTTGTACTATTTTACGTTTAGCTGAT | |
| | GATATGGGTACATCATTAGATGAAGTTGAACGTGGCGATGTTCCTAA | |
| | ATCTGTACAATGCTATATGAATGAGAAAAACGCCTCTGAAGAAGAA | |
| | GCACGTGAACATGTTCGTAGTTTAATTGATCAGACATGGAAGATGAT | |
| | GAATAAAGAAATGATGACTTCATCATTTTCAAAATACTTCGTACAAG | |
| | TGTCTGCAAATCTTGCTCGTATGGCACAATGGATATATCAACATGAA | |
| | AGTGATGGTTTCGGTATGCAACACTCTTTAGTTAACAAAATGCTTCG | |
| | TGGTTTACTTTTTGACCGTTATGAAGGTACCGGTGAAAACTTATACT | |
| | TTCAAGGCTCAGGTGGCGGTGGAAGTGATTACAAAGATGATGATGA | |
| | TAAAGGAACCGGTTAATCTAGACTCGAG | |
| 129 | CATATGGTACCACGTCGCATGGGTGATTTTCATTCAAACTTATGGGA | Pinene (A. grandis) |
| | TGATGATGTAATTCAATCTTTACCCACAGCTTACGAAGAAAAATCTT | |
| | ATCTTGAACGTGCTGAGAAGTTAATTGGAGAAGTTGAAAATATGTTC | |
| | AACAGTATGAGTTTAGAAGATGGTGAACTTATGAGTCCATTAAATG | |
| | ATTTAATTCAACGCCTTTGGATTGTTGATTCTTTAGGTAGATTAGGTA | |
| | TCCATCGTCACTTTAAAGATGAGATTAAAAGTGCTTTAGATTATGTT | |
| | TACAGTTACTGGGGTGAAAACGGAATAGGTTGTGGTCGTGAAAGTG | |
| | CTGTAACTGATTTAAACAGTACAGCTTTAGGCTTTCGTACACTTCGTT | |
| | TACACGGTTATCCAGTTTCATCTGATGTATTTAAAGCATTTAAAGGT | |
| | CAAAATGGTCAATTCAGTTGTTCAGAAAATATCCAAACAGATGAAG | |
| | AAATTCGTGGTGTTCTTAACTTATTTAGAGCCAGTTTAATAGCCTTCC | |
| | CTGGTGAGAAAATAATGGACGAAGCTGAAATCTTCTCTACAAAATA | |
| | CTTAAAGGAAGCATTACAAAAGATCCCAGTTAGTTCATTATCACGTG | |
| | AAATCGGTGATGTACTTGAATATGGATGGCATACATACTTACCACGT | |
| | TTAGAAGCACGTAACTATATTCATGTTTTCGGACAAGATACAGAGAA | |
| | TACAAAAAGTTATGTAAAATCAAAGAAACTTTTAGAATTAGCTAAA | |
| | TTAGAATTTAACATTTTTCAGAGCTTACAAAAACGTGAATTAGAAAG | |
| | CCTTGTTCGTTGGTGGAAAGAATCTGGATTTCCTGAAATGACATTCT | |
| | GTAGACACAGACACGTGGAATATTACACACTTGCATCATGTATTGCA | |
| | TTCGAACCTCAGCATAGTGGTTTTCGTTTAGGTTTTGCTAAAACATGT | |
| | CACCTTATAACAGTTTTAGATGACATGTATGACACTTTCGGCACCGT | |
| | AGACGAATTAGAGTTATTTACAGCAACTATGAAACGTTGGGACCCA | |
| | AGTTCAATTGACTGCCTTCCAGAATACATGAAAGGAGTTTACATTGC | |
| | TGTGTATGATACAGTTAATGAAATGGCTCGTGAAGCTGAGGAAGCT | |
| | CAAGGTCGCGATACACTTACATACGCTCGTGAGGCCTGGGAGGCTT | |
| | ATATAGATTCTTATATGCAAGAAGCTCGCTGGATTGCTACTGGATAC | |
| | TTACCTTCTTTCGATGAATATTATGAAAATGGTAAGGTTTCATGTGG | |
| | TCACCGTATATCTGCTTTACAACCAATTCTTACTATGGATATTCCATT | |
| | TCCAGATCACATTTTAAAGGAAGTTGACTTTCCTTCTAAACTTAATG | |
| | ACTTAGCTTGTGCTATCTTACGCCTTCGCGGTGATACTCGTTGTTACA | |
| | AAGCAGACCGTGCACGTGGTGAAGAGGCTAGTTCTATTTCTTGTTAT | |
| | ATGAAAGATAATCCAGGTGTTTCTGAAGAAGATGCCTTAGATCATAT | |
| | TAACGCAATGATCAGTGATGTTATTAAGGGCTTAAACTGGGAATTAC | |
| | TTAAACCCGACATTAACGTACCTATTTCTGCTAAGAAACATGCTTTC | |
| | GACATTGCTCGTGCTTTTCACTACGGTTATAAATATCGTGATGGCTA | |
| | TTCAGTTGCTAATGTTGAAACAAAATCTTTAGTTACACGTACTTTACT | |
| | TGAATCAGTTCCATTAGGTACCGGTGAAAACTTATACTTTCAAGGCT | |
| | CAGGTGGCGGTGGAAGTGATTACAAAGATGATGATGATAAAGGAAC | |
| | CGGTTAATCTAGACTCGAG | |
| 130 | CATATGGTACCACGTAGAGTTGGTAATTATCATTCTAATCTTTGGGA | Camphene |
| | TGATGATTTTATACAAAGTTTAATTTCTACACCTTACGGTGCTCCTGA | (A. grandis) |
| | CTACCGTGAACGCGCTGATCGTCTTATTGGTGAAGTAAAAGATATTA | |
| | TGTTTAATTTCAAATCTTTAGAGGATGGTGGTAATGACTTATTACAA | |
| | CGTTTACTTTTAGTTGATGACGTAGAACGTTTAGGCATTGATCGTCA | |
| | TTTCAAAAAGGAAATTAAGACTGCATTAGATTATGTAAATAGTTATT | |
| | GGAATGAAAAAGGAATTGGTTGTGGTCGTGAGTCTGTAGTTACAGA | |
| | CTTAAATTCAACTGCTTTAGGCCTTCGTACCTTAAGATTACATGGTTA | |
| | TACTGTTAGCTCTGACGTTTTAAATGTTTTTAAAGATAAAAATGGTC | |
| | AATTTTCATCTACAGCTAATATTCAAATTGAAGGTGAAATTCGTGGT | |
| | GTTTTAAATCTTTTTCGTGCCTCTCTTGTAGCTTTTCCAGGTGAGAAA | |
| | GTGATGGATGAGGCTGAAACTTTTTCAACAAAATATCTTCGTGAAGC | |
| | ATTACAGAAAATTCCTGCCAGTTCAATTTTATCATTAGAAATACGTG | |
| | ATGTATTAGAATATGGATGGCATACTAATTTACCACGTTTAGAAGCA | |
| | CGTAATTACATGGATGTTTTCGGTCAGCACACCAAGAACAAAAATG | |
| | CAGCCGAAAAATTACTTGAATTAGCAAAATTAGAGTTCAATATCTTT | |
| | CACAGCTTACAAGAACGTGAATTAAAGCACGTTTCAAGATGGTGGA | |
| | AAGACTCTGGTAGTCCAGAGATGACTTTCTGTCGCCACCGCCATGTG | |
| | GAATATTATGCTTTAGCTTCTTGTATTGCTTTCGAACCCCAGCACAGT | |
| | GGTTTCCGTTTAGGTTTTACTAAAATGAGTCATTTAATCACAGTGTTA | |
| | GATGATATGTATGATGTATTCGGTACAGTTGATGAATTAGAGTTATT | |
| | TACCGCCACTATTAAACGTTGGGACCCTTCTGCTATGGAATGTTTAC | |
| | CAGAGTACATGAAAGGTGTTTACATGATGGTTTATCATACAGTTAAC | |
| | GAAATGGCTCGTGTGGCAGAAAAGGCTCAAGGTAGAGACACATTAA | |
| | ACTATGCTCGTCAAGCCTGGGAAGCATGTTTTGACTCTTATATGCAA | |
| | GAAGCAAAATGGATTGCAACAGGTTACTTACCTACATTCGAGGAAT | |
| | ATTTAGAAAATGGTAAAGTGAGTTCAGCACATCGTCCTTGTGCATTA | |
| | CAACCTATTTTAACTCTTGATATTCCATTTCCCGATCATATTCTTAAA | |
| | GAAGTGGATTTCCCAAGCAAACTTAATGACTTAATTTGTATTATCTT | |
| | ACGTCTTAGAGGAGACACACGTTGCTATAAAGCAGACCGTGCCCGT | |
| | GGTGAAGAAGCATCATCAATATCTTGTTATATGAAAGATAACCCAG | |
| | GTTTAACTGAAGAAGATGCTTTAAACCACATTAACTTTATGATTCGT | |
| | GACGCAATCCGCGAATTAAACTGGGAGTTACTTAAACCAGATAATA | |
| | GTGTTCCAATTACTTCAAAGAAACATGCTTTTGATATTTCACGTGTGT | |
| | GGCACCACGGATACCGTTATCGTGATGGTTACAGCTTTGCAAACGTG | |
| | GAAACTAAAAGTCTTGTAATGCGTACTGTAATAGAACCAGTACCATT | |
| | AGGTACCGGTGAAAACTTATACTTTCAAGGCTCAGGTGGCGGTGGA | |
| | AGTGATTACAAAGATGATGATGATAAAGGAACCGGTTAATCTAGAC | |
| | TCGAG | |
| 131 | CATATGGTACCACGTCGTTCAGGAGATTATCAACCAAGTTTATGGGA | Sabinene (S. officinalis) |
| | CTTTAATTACATTCAATCTTTAAACACACCTTACAAAGAACAACGTC | |
| | ATTTTAATCGTCAAGCTGAGTTAATTATGCAAGTTCGTATGTTATTA | |
| | AAGGTAAAAATGGAAGCAATTCAACAATTAGAGTTAATAGATGATT | |
| | TACAGTACTTAGGATTATCATATTTCTTTCAAGACGAAATTAAACAA | |
| | ATCTTAAGCTCTATTCACAATGAACCTCGTTATTTTCATAATAATGAC | |
| | CTTTATTTCACTGCTTTAGGTTTTAGAATTTTACGTCAACATGGTTTT | |
| | AATGTTTCAGAAGACGTATTTGACTGCTTTAAAATCGAAAAATGTTC | |
| | TGACTTTAATGCTAACTTAGCTCAGGACACAAAGGGTATGTTACAAT | |
| | TATATGAAGCTAGTTTCTTATTAAGAGAAGGAGAAGATACACTTGA | |
| | ATTAGCTCGTCGTTTTAGTACACGTTCTTTACGTGAAAAATTTGATG | |
| | AAGGTGGTGACGAGATAGATGAAGATTTAAGTAGTTGGATTCGTCA | |
| | TTCTTTAGATTTACCATTACACTGGCGTGTTCAAGGTTTAGAAGCTC | |
| | GTTGGTTTTTAGATGCCTATGCTCGTCGTCCAGATATGAACCCTCTTA | |
| | TTTTCAAATTAGCTAAATTAAATTTTAACATTGTTCAAGCTACATACC | |
| | AAGAAGAATTAAAAGACATCTCTCGTTGGTGGAACAGTAGTTGTTTA | |
| | GCAGAGAAATTACCCTTCGTTCGCGATCGTATTGTAGAATGTTTCTT | |
| | CTGGGCTATTGCTGCTTTCGAACCACACCAATACTCATATCAACGTA | |
| | AAATGGCCGCTGTAATTATTACATTTATTACTATTATTGATGATGTTT | |
| | ACGATGTATATGGTACTATTGAAGAATTAGAGTTATTAACAGATATG | |
| | ATTCGTAGATGGGATAATAAGAGTATTAGTCAACTTCCTTACTATAT | |
| | GCAAGTTTGTTATTTAGCTCTTTATAACTTCGTAAGTGAACGCGCAT | |
| | ACGACATCTTAAAAGATCAACACTTTAACAGTATTCCATACCTTCAA | |
| | AGAAGTTGGGTTTCATTAGTTGAGGGATACTTAAAAGAAGCATATTG | |
| | GTACTATAACGGTTACAAACCAAGTCTTGAAGAATATCTTAATAATG | |
| | CAAAAATTAGTATTAGTGCACCCACCATTATTTCACAATTATACTTT | |
| | ACTTTAGCAAATAGTATCGACGAAACTGCCATTGAAAGTTTATACCA | |
| | ATATCACAACATTTTATACTTATCAGGTACTATCTTACGTTTAGCTGA | |
| | TGATTTAGGAACTTCACAACATGAATTAGAACGTGGTGATGTTCCCA | |
| | AAGCTATTCAATGTTATATGAATGATACAAATGCATCAGAAAGAGA | |
| | AGCTGTAGAACATGTTAAATTTCTTATTCGTGAAGCCTGGAAAGAAA | |
| | TGAATACAGTTACTACCGCATCAGATTGTCCTTTTACAGACGATCTT | |
| | GTTGCCGCCGCAGCTAATTTAGCTCGTGCTGCTCAATTCATTTACTTA | |
| | GATGGTGATGGTCATGGTGTACAACATAGCGAAATTCATCAGCAAA | |
| | TGGGCGGTCTTCTTTTTCAACCATACGTTGGTACCGGTGAAAACTTA | |
| | TACTTTCAAGGCTCAGGTGGCGGTGGAAGTGATTACAAAGATGATG | |
| | ATGATAAAGGAACCGGTTAATCTAGACTCGAG | |
| 132 | CATATGGTACCACGCAGAATTGGTGATTACCATAGTAACATTTGGGA | Myrcene (A. grandis) |
| | TGATGATTTTATCCAGTCACTTTCTACCCCTTATGGTGAACCATCTTA | |
| | CCAAGAAAGAGCTGAACGTCTTATTGTAGAAGTGAAAAAGATTTTC | |
| | AACAGTATGTACTTAGATGACGGTCGTTTAATGAGTTCTTTTAATGA | |
| | CTTAATGCAACGTTTATGGATTGTAGACTCAGTAGAACGTTTAGGTA | |
| | TTGCCCGTCACTTCAAAAATGAAATTACATCTGCCCTTGACTATGTTT | |
| | TTCGTTATTGGGAAGAAAACGGTATAGGTTGTGGTCGTGATTCTATT | |
| | GTAACTGACTTAAATAGCACAGCTTTAGGTTTTCGTACACTTCGTTT | |
| | ACACGGTTATACAGTTTCTCCAGAGGTTTTAAAAGCATTTCAAGATC | |
| | AAAATGGTCAATTCGTTTGTTCACCAGGACAAACAGAAGGTGAAAT | |
| | TCGTTCAGTTTTAAATTTATATCGTGCAAGTTTAATTGCCTTTCCAGG | |
| | CGAAAAAGTTATGGAAGAAGCAGAAATCTTCTCTACTCGCTATTTAA | |
| | AAGAAGCTCTTCAAAAGATTCCAGTTAGCGCATTATCACAAGAAAT | |
| | CAAATTTGTTATGGAATATGGATGGCATACAAATTTACCTAGATTAG | |
| | AAGCACGTAACTATATTGATACTTTAGAAAAGGATACATCAGCTTGG | |
| | TTAAACAAAAATGCAGGTAAAAAGTTATTAGAATTAGCTAAATTAG | |
| | AATTTAACATCTTTAACTCATTACAACAAAAAGAATTACAATACTTA | |
| | CTTCGCTGGTGGAAAGAATCTGACTTACCTAAATTAACCTTTGCACG | |
| | TCATAGACACGTTGAATTTTACACATTAGCTTCTTGTATTGCTATTGA | |
| | TCCCAAACATTCAGCATTCCGTTTAGGATTCGCTAAAATGTGTCACT | |
| | TAGTTACAGTTCTTGACGATATTTATGATACTTTCGGTACTATTGATG | |
| | AACTTGAGTTATTTACTTCTGCAATTAAACGTTGGAATAGTTCTGAA | |
| | ATTGAACACTTACCAGAATATATGAAATGCGTGTATATGGTTGTTTT | |
| | TGAAACTGTTAATGAATTAACTCGTGAAGCTGAGAAAACACAAGGA | |
| | CGTAACACTTTAAACTATGTTCGTAAAGCATGGGAAGCATATTTTGA | |
| | TTCTTATATGGAGGAAGCAAAGTGGATCTCAAACGGATATTTACCAA | |
| | TGTTTGAAGAATACCACGAAAATGGTAAAGTGTCATCTGCATACCGT | |
| | GTAGCAACATTACAACCAATTTTAACTTTAAACGCTTGGTTACCCGA | |
| | CTACATTCTTAAAGGAATTGATTTCCCAAGTCGTTTTAACGATTTAG | |
| | CTAGTTCATTCTTACGTTTACGTGGCGATACTCGCTGTTACAAAGCT | |
| | GACCGTGATCGTGGTGAAGAAGCTAGCTGCATTTCTTGTTACATGAA | |
| | AGATAATCCAGGTTCTACCGAAGAAGATGCACTTAATCACATTAAC | |
| | GCTATGGTAAATGACATCATTAAAGAATTAAACTGGGAATTATTACG | |
| | CAGTAATGATAATATTCCTATGTTAGCTAAAAAGCACGCTTTTGATA | |
| | TTACTCGTGCACTTCACCACTTATACATTTATCGCGATGGTTTCAGTG | |
| | TTGCTAATAAAGAAACTAAAAAGTTAGTTATGGAGACATTACTTGA | |
| | ATCAATGTTATTTGGTACCGGTGAAAACTTATACTTTCAAGGCTCAG | |
| | GTGGCGGTGGAAGTGATTACAAAGATGATGATGATAAAGGAACCGG | |
| | TTAATCTAGACTCGAG | |
| 133 | CATATGGTACCACAATCTGCTGAAAAGAACGACTCTTTATCAAGTTC | Abietadiene |
| | TACATTAGTTAAGAGAGAATTTCCACCCGGTTTCTGGAAAGACGACT | (A. grandis) |
| | TAATCGACAGTTTAACTTCAAGTCACAAAGTAGCTGCTAGCGATGAA | |
| | AAACGTATCGAAACCTTAATTTCAGAAATTAAGAATATGTTTCGTTG | |
| | TATGGGTTATGGTGAGACAAATCCATCAGCTTATGATACTGCTTGGG | |
| | TAGCTCGCATCCCAGCAGTTGATGGATCAGATAATCCTCACTTTCCA | |
| | GAGACTGTGGAATGGATCTTACAAAATCAATTAAAAGATGGTTCTTG | |
| | GGGTGAAGGTTTTTACTTCCTTGCTTATGATCGCATTTTAGCCACTTT | |
| | AGCTTGTATTATCACACTTACACTTTGGCGTACTGGAGAAACACAAG | |
| | TACAGAAAGGTATCGAATTTTTCCGCACTCAAGCAGGTAAAATGGA | |
| | AGATGAAGCAGATTCACACCGTCCAAGTGGTTTTGAGATTGTATTTC | |
| | CTGCTATGTTAAAAGAGGCTAAGATTTTAGGCTTAGATTTACCTTAT | |
| | GATCTTCCTTTTCTTAAACAAATTATTGAAAAGAGAGAAGCTAAGTT | |
| | AAAACGTATTCCTACAGATGTTTTATATGCTTTACCAACTACTTTACT | |
| | TTATTCATTAGAAGGTTTACAAGAAATAGTAGACTGGCAAAAAATC | |
| | ATGAAATTACAAAGTAAAGATGGTAGTTTCTTATCTTCTCCTGCCTC | |
| | AACAGCAGCAGTATTTATGAGAACAGGTAACAAAAAGTGTTTAGAT | |
| | TTCTTAAATTTCGTGCTTAAAAAGTTCGGTAATCATGTTCCATGCCAC | |
| | TATCCTTTAGACCTTTTTGAGCGTCTTTGGGCAGTTGATACTGTTGAA | |
| | AGATTAGGTATTGACCGTCATTTTAAAGAAGAAATAAAAGAGGCTT | |
| | TAGACTATGTGTATTCACACTGGGACGAACGTGGTATTGGTTGGGCT | |
| | CGTGAAAACCCCGTTCCAGATATTGACGATACAGCAATGGGTCTTCG | |
| | TATTTTACGTCTTCATGGTTACAATGTTAGCAGCGATGTTCTTAAAAC | |
| | ATTTCGTGATGAAAATGGTGAGTTCTTTTGCTTTTTAGGACAAACAC | |
| | AAAGAGGTGTGACTGATATGTTAAATGTTAATCGTTGTAGCCATGTA | |
| | TCTTTCCCTGGTGAAACTATAATGGAAGAGGCAAAATTATGTACTGA | |
| | ACGTTACTTACGCAACGCATTAGAAAATGTAGACGCTTTTGATAAGT | |
| | GGGCATTTAAGAAAAACATTCGTGGTGAGGTAGAATATGCTCTTAA | |
| | ATATCCTTGGCATAAATCAATGCCACGTTTAGAAGCACGTTCATATA | |
| | TTGAAAATTACGGTCCAGATGATGTTTGGTTAGGTAAAACTGTTTAT | |
| | ATGATGCCTTACATTTCAAATGAAAAGTACTTAGAGTTAGCTAAACT | |
| | TGATTTTAACAAAGTTCAGTCAATCCACCAGACAGAACTTCAAGACT | |
| | TACGCCGTTGGTGGAAAAGTTCTGGTTTTACAGATTTAAACTTTACA | |
| | AGAGAACGTGTTACTGAAATTTACTTTTCACCTGCATCTTTTATCTTC | |
| | GAACCAGAATTTAGTAAATGTCGTGAGGTTTATACAAAAACTTCTAA | |
| | TTTTACTGTAATTTTAGACGATTTATATGACGCTCATGGCTCTTTAGA | |
| | TGACTTAAAACTTTTTACAGAGAGTGTTAAACGTTGGGATTTATCTT | |
| | TAGTTGACCAAATGCCCCAGCAGATGAAAATCTGTTTTGTAGGTTTC | |
| | TATAATACATTCAACGATATTGCTAAAGAAGGTAGAGAACGTCAAG | |
| | GTCGTGATGTTTTAGGTTATATTCAAAACGTATGGAAAGTACAACTT | |
| | GAAGCATATACTAAAGAAGCAGAATGGTCAGAAGCAAAATATGTTC | |
| | CTAGTTTTAACGAATACATTGAAAATGCTTCAGTTTCAATTGCCTTA | |
| | GGTACAGTAGTACTTATCAGTGCTTTATTTACCGGAGAAGTTTTAAC | |
| | AGATGAAGTTTTATCTAAAATTGACCGTGAAAGTAGATTCTTACAGT | |
| | TAATGGGCTTAACTGGACGTTTAGTAAATGATACTAAAACATATCAA | |
| | GCTGAGCGTGGTCAAGGTGAAGTTGCTAGTGCAATTCAATGTTATAT | |
| | GAAAGACCACCCTAAAATTAGTGAAGAAGAAGCATTACAACATGTA | |
| | TATTCTGTAATGGAAAATGCATTAGAAGAATTAAATCGTGAGTTCGT | |
| | TAACAACAAAATTCCAGACATCTATAAACGTCTTGTTTTCGAAACTG | |
| | CACGTATAATGCAATTATTTTACATGCAAGGTGATGGTTTAACATTA | |
| | AGTCACGATATGGAAATTAAAGAGCACGTAAAGAATTGTTTATTCC | |
| | AGCCAGTAGCTGGTACCGGTGAAAACTTATACTTTCAAGGCTCAGGT | |
| | GGCGGTGGAAGTGATTACAAAGATGATGATGATAAAGGAACCGGTT | |
| | AATCTAGACTCGAG | |
| 134 | CATATGGTACCATCTTCATCAACAGGCACTTCAAAAGTAGTAAGCGA | Taxadiene |
| | AACATCTTCAACTATTGTAGACGATATTCCACGTCTTTCAGCAAATT | (T. brevifolia) |
| | ATCATGGTGATTTATGGCATCACAACGTAATTCAGACTTTAGAAACA | |
| | CCATTTAGAGAAAGTTCAACTTATCAAGAGCGTGCAGATGAATTAGT | |
| | AGTGAAAATCAAAGATATGTTCAATGCATTAGGTGACGGTGACATC | |
| | TCACCTTCAGCTTATGATACTGCATGGGTAGCTCGTGTTGCTACCATT | |
| | TCTTCTGATGGTAGCGAAAAACCACGTTTTCCTCAAGCTCTTAATTG | |
| | GGTTTTTAACAATCAATTACAAGATGGATCATGGGGTATTGAATCAC | |
| | ATTTTAGTTTATGCGATCGTTTACTTAATACTACAAATTCAGTTATTG | |
| | CTTTATCAGTATGGAAAACTGGTCACTCACAGGTTCAACAAGGTGCC | |
| | GAATTTATTGCTGAAAATTTACGTCTTTTAAATGAAGAAGACGAATT | |
| | AAGTCCTGATTTTCAAATTATCTTCCCAGCTTTATTACAGAAAGCCA | |
| | AGGCTTTAGGAATCAATTTACCCTATGATTTACCATTCATCAAATAT | |
| | CTTAGTACAACACGCGAAGCTCGTTTAACAGATGTGTCAGCTGCTGC | |
| | TGACAACATACCAGCCAATATGCTTAATGCACTTGAAGGTTTAGAAG | |
| | AAGTGATTGATTGGAATAAAATCATGCGTTTTCAATCTAAAGATGGT | |
| | TCATTTTTATCTTCTCCAGCTAGTACAGCCTGTGTTTTAATGAATACA | |
| | GGTGATGAAAAATGTTTCACATTCTTAAATAACTTATTAGATAAATT | |
| | CGGCGGTTGTGTTCCATGTATGTATAGCATTGATTTATTAGAACGTTT | |
| | ATCTTTAGTGGACAACATTGAACACTTAGGTATTGGTCGTCACTTTA | |
| | AACAAGAAATCAAAGGTGCATTAGATTATGTATATCGTCATTGGTCT | |
| | GAACGCGGTATCGGTTGGGGTAGAGACTCTTTAGTTCCAGATTTAAA | |
| | CACCACAGCTTTAGGTTTACGCACATTAAGAATGCACGGTTATAACG | |
| | TGTCTAGTGATGTACTTAACAATTTCAAAGACGAAAATGGTCGTTTC | |
| | TTTAGTAGTGCTGGTCAAACACACGTAGAGTTACGTTCTGTTGTAAA | |
| | TCTTTTTCGCGCCTCAGATTTAGCCTTTCCAGACGAACGTGCAATGG | |
| | ATGATGCTCGTAAATTCGCAGAACCATATTTACGTGAAGCATTAGCT | |
| | ACAAAAATATCAACAAATACAAAGTTATTCAAAGAAATTGAATATG | |
| | TTGTTGAATACCCTTGGCACATGTCAATTCCACGTTTAGAAGCTCGT | |
| | AGTTATATTGACAGTTATGATGATAATTATGTATGGCAACGTAAGAC | |
| | TTTATATCGTATGCCATCATTAAGTAATTCAAAATGTTTAGAACTTG | |
| | CTAAATTAGATTTCAATATTGTTCAATCTTTACACCAAGAAGAACTT | |
| | AAACTTTTAACTCGTTGGTGGAAAGAATCTGGTATGGCAGACATAA | |
| | ATTTCACCCGCCATCGTGTAGCTGAAGTTTACTTTTCTAGTGCTACAT | |
| | TTGAGCCAGAATATAGTGCTACTCGTATTGCATTCACAAAAATTGGT | |
| | TGCTTACAAGTACTTTTCGATGATATGGCTGACATTTTCGCCACTTTA | |
| | GATGAGTTAAAAAGTTTTACTGAAGGTGTTAAACGCTGGGACACAT | |
| | CATTATTACATGAAATTCCCGAATGTATGCAAACTTGTTTTAAAGTA | |
| | TGGTTTAAACTTATGGAAGAAGTAAACAACGACGTAGTAAAAGTTC | |
| | AAGGAAGAGATATGTTAGCACATATTCGTAAACCCTGGGAATTATA | |
| | CTTTAATTGTTATGTTCAAGAACGTGAATGGTTAGAAGCTGGTTATA | |
| | TTCCTACATTCGAAGAATATCTTAAAACTTATGCTATTAGTGTAGGC | |
| | CTTGGTCCTTGTACCTTACAACCTATTCTTTTAATGGGTGAGTTAGTT | |
| | AAAGATGATGTAGTAGAAAAAGTTCATTACCCTTCTAACATGTTCGA | |
| | ATTAGTTTCTTTAAGCTGGCGTTTAACTAATGATACCAAAACATATC | |
| | AAGCAGAAAAAGTACGCGGTCAACAAGCTAGTGGCATTGCCTGTTA | |
| | TATGAAAGACAATCCAGGTGCTACTGAAGAAGATGCTATTAAACAC | |
| | ATTTGTCGTGTTGTTGATCGTGCATTAAAAGAAGCAAGTTTCGAATA | |
| | TTTCAAGCCTTCAAATGACATTCCTATGGGTTGTAAATCTTTTATCTT | |
| | TAACTTACGTTTATGTGTACAAATTTTCTATAAATTCATTGATGGTTA | |
| | TGGTATCGCAAACGAAGAAATTAAGGACTACATTCGTAAGGTTTAT | |
| | ATTGATCCAATTCAAGTTGGTACCGGTGAAAACTTATACTTTCAAGG | |
| | CTCAGGTGGCGGTGGAAGTGATTACAAAGATGATGATGATAAAGGA | |
| | ACCGGTTAATCTAGACTCGAG | |
| 135 | CATATGGTACCACACAAGTTCACAGGTGTTAACGCTAAATTCCAGCA | FPP (G. gallus) |
| | ACCAGCATTAAGAAATTTATCTCCAGTGGTAGTTGAGCGCGAACGTG | |
| | AGGAATTTGTAGGATTCTTTCCACAAATTGTTCGTGACTTAACTGAA | |
| | GATGGTATTGGTCATCCAGAAGTAGGTGACGCTGTAGCTCGTCTTAA | |
| | AGAAGTATTACAATACAACGCACCTGGTGGTAAATGCAATAGAGGT | |
| | TTAACAGTTGTTGCAGCTTACCGTGAACTTTCTGGACCAGGTCAAAA | |
| | AGACGCTGAAAGTCTTCGTTGTGCTTTAGCAGTAGGATGGTGTATTG | |
| | AATTATTCCAAGCCTTTTTCTTAGTTGCTGACGATATAATGGACCAG | |
| | TCATTAACTAGACGTGGTCAATTATGTTGGTACAAGAAAGAAGGTGT | |
| | TGGTTTAGATGCAATAAATGATTCTTTTCTTTTAGAAAGCTCTGTGTA | |
| | TCGCGTTCTTAAAAAGTATTGCCGTCAACGTCCATATTATGTACATTT | |
| | ATTAGAGCTTTTTCTTCAAACAGCTTACCAAACAGAATTAGGACAAA | |
| | TGTTAGATTTAATCACTGCTCCTGTATCTAAGGTAGATTTAAGCCATT | |
| | TCTCAGAAGAACGTTACAAAGCTATTGTTAAGTATAAAACTGCTTTC | |
| | TATTCATTCTATTTACCAGTTGCAGCAGCTATGTATATGGTTGGTATA | |
| | GATTCTAAAGAAGAACATGAAAACGCAAAAGCTATTTTACTTGAGA | |
| | TGGGTGAATACTTCCAAATTCAAGATGATTATTTAGATTGTTTTGGC | |
| | GATCCTGCTTTAACAGGTAAAGTAGGTACTGATATTCAAGATAACAA | |
| | ATGTTCATGGTTAGTTGTGCAATGCTTACAAAGAGTAACACCAGAAC | |
| | AACGTCAACTTTTAGAAGATAATTACGGTCGTAAAGAACCAGAAAA | |
| | AGTTGCTAAAGTTAAAGAATTATATGAGGCTGTAGGTATGAGAGCC | |
| | GCCTTTCAACAATACGAAGAAAGTAGTTACCGTCGTCTTCAAGAGTT | |
| | AATTGAGAAACATTCTAATCGTTTACCAAAAGAAATTTTCTTAGGTT | |
| | TAGCTCAGAAAATATACAAACGTCAAAAAGGTACCGGTGAAAACTT | |
| | ATACTTTCAAGGCTCAGGTGGCGGTGGAAGTGATTACAAAGATGAT | |
| | GATGATAAAGGAACCGGTTAATCTAGACTCGAG | |
| 136 | CATATGGTACCATCATTAACTGAAGAAAAACCAATTCGCCCAATCGC | Amorphadiene |
| | AAACTTTCCTCCAAGCATTTGGGGAGATCAATTCTTAATTTACGAAA | (A. annua) |
| | AACAAGTAGAACAAGGTGTTGAACAGATTGTTAACGACCTTAAGAA | |
| | AGAAGTGCGCCAACTTTTAAAAGAGGCTTTAGATATTCCAATGAAA | |
| | CACGCAAACCTTTTAAAACTTATTGACGAAATTCAACGTCTTGGTAT | |
| | TCCATATCACTTTGAACGTGAAATTGATCATGCATTACAATGTATCT | |
| | ATGAAACTTATGGTGATAATTGGAATGGTGATCGTTCTTCATTATGG | |
| | TTCCGTTTAATGCGTAAACAAGGTTATTATGTTACATGTGACGTGTTT | |
| | AACAATTACAAAGATAAAAATGGTGCATTTAAACAATCTTTAGCTA | |
| | ATGATGTTGAAGGTTTATTAGAATTATATGAAGCTACTTCAATGCGT | |
| | GTTCCAGGTGAAATTATTCTTGAAGATGCATTAGGTTTTACACGTTC | |
| | TCGTTTATCTATTATGACAAAAGACGCATTTAGTACAAATCCTGCTT | |
| | TATTTACTGAAATTCAGCGTGCCCTTAAACAGCCTTTATGGAAACGT | |
| | TTACCAAGAATTGAAGCTGCTCAATATATTCCATTTTATCAACAACA | |
| | AGATTCTCACAATAAGACATTACTTAAATTAGCCAAATTAGAATTTA | |
| | ATCTTTTACAATCATTACATAAAGAAGAATTAAGTCATGTGTGTAAA | |
| | TGGTGGAAAGCATTTGATATTAAGAAGAATGCTCCATGTTTACGTGA | |
| | TAGAATTGTAGAGTGTTACTTTTGGGGCCTTGGTAGTGGTTACGAGC | |
| | CACAATATTCACGTGCTCGTGTATTCTTTACAAAAGCTGTTGCAGTT | |
| | ATTACTTTAATTGACGATACCTATGATGCATACGGAACCTATGAGGA | |
| | GCTTAAAATTTTCACTGAAGCTGTAGAACGTTGGTCTATAACTTGTT | |
| | TAGATACTTTACCAGAATATATGAAACCCATCTACAAATTATTCATG | |
| | GACACATACACTGAAATGGAAGAATTTTTAGCAAAAGAAGGTCGCA | |
| | CAGACCTTTTTAACTGTGGTAAAGAATTTGTTAAAGAGTTTGTTCGT | |
| | AACTTAATGGTAGAAGCTAAGTGGGCTAATGAAGGTCACATTCCTA | |
| | CTACAGAAGAGCACGATCCAGTAGTAATAATTACAGGTGGAGCAAA | |
| | CTTACTTACCACAACTTGTTACTTAGGTATGTCTGACATTTTTACAAA | |
| | AGAATCAGTAGAGTGGGCAGTATCTGCACCACCATTATTCCGTTATT | |
| | CTGGCATACTTGGTCGTCGTCTTAATGATTTAATGACTCATAAAGCT | |
| | GAACAAGAGCGTAAACACTCATCAAGTAGTTTAGAAAGCTATATGA | |
| | AGGAATATAACGTTAACGAAGAGTATGCTCAAACACTTATTTACAA | |
| | AGAGGTTGAAGACGTTTGGAAGGACATTAACCGTGAATACTTAACA | |
| | ACTAAAAACATTCCACGTCCTCTTTTAATGGCTGTAATATACTTATGT | |
| | CAATTCTTAGAAGTACAATACGCTGGAAAAGATAACTTTACACGTAT | |
| | GGGTGATGAATATAAACACTTAATAAAGAGTTTATTAGTTTATCCTA | |
| | TGTCAATAGGTACCGGTGAAAACTTATACTTTCAAGGCTCAGGTGGC | |
| | GGTGGAAGTGATTACAAAGATGATGATGATAAAGGAACCGGTTAAT | |
| | CTAGACTCGAG | |
| 137 | CATATGGTACCAGCAGGTGTATCAGCTGTGTCAAAAGTTTCTTCATT | Bisabolene |
| | AGTATGTGACTTAAGTAGTACTAGCGGCTTAATTCGTAGAACTGCAA | (A. grandis) |
| | ATCCTCACCCTAATGTATGGGGTTATGACTTAGTTCATTCTTTAAAAT | |
| | CTCCATATATTGATAGTAGCTATCGTGAACGTGCTGAAGTGCTTGTA | |
| | AGTGAAATAAAAGCTATGTTAAATCCAGCAATTACTGGAGATGGTG | |
| | AATCAATGATTACACCTTCAGCTTATGACACTGCTTGGGTTGCACGT | |
| | GTACCAGCAATTGATGGTAGCGCACGTCCACAATTTCCACAAACAGT | |
| | AGATTGGATTTTAAAGAATCAATTAAAAGATGGTTCTTGGGGTATTC | |
| | AATCACACTTTTTACTTTCAGACCGTTTATTAGCTACTCTTAGCTGTG | |
| | TTTTAGTTTTACTTAAATGGAATGTTGGTGATTTACAGGTTGAGCAA | |
| | GGTATTGAGTTTATTAAGTCAAACCTTGAATTAGTAAAAGATGAAAC | |
| | TGATCAAGATTCTTTAGTGACTGATTTTGAGATTATTTTCCCTAGCTT | |
| | ACTTCGTGAGGCCCAAAGTTTACGTTTAGGTCTTCCATACGATTTAC | |
| | CTTACATCCACTTATTACAAACAAAACGTCAGGAACGTTTAGCAAAA | |
| | TTAAGCCGTGAAGAAATATATGCAGTTCCAAGTCCACTTTTATATTC | |
| | TTTAGAGGGTATTCAAGATATTGTTGAGTGGGAACGTATTATGGAAG | |
| | TACAATCTCAGGATGGATCATTTTTAAGTTCTCCAGCATCAACCGCA | |
| | TGTGTTTTTATGCATACAGGTGACGCTAAGTGTTTAGAATTTCTTAAC | |
| | AGTGTAATGATTAAGTTTGGTAATTTTGTACCATGCCTTTATCCTGTA | |
| | GATTTATTAGAACGTTTACTTATAGTAGATAATATAGTTCGTCTTGGT | |
| | ATTTACCGTCACTTCGAAAAAGAAATTAAAGAAGCATTAGATTATGT | |
| | ATATCGCCATTGGAATGAACGTGGTATTGGTTGGGGTCGTTTAAATC | |
| | CAATTGCTGACTTAGAAACAACTGCTTTAGGTTTTCGTTTATTACGTT | |
| | TACACCGTTATAATGTATCTCCAGCAATCTTTGATAATTTCAAAGAT | |
| | GCCAATGGCAAATTCATTTGTAGCACTGGTCAGTTTAATAAGGATGT | |
| | GGCTTCAATGTTAAACTTATACCGTGCATCACAATTAGCATTCCCAG | |
| | GCGAAAACATTTTAGATGAAGCTAAATCTTTTGCCACCAAATACTTA | |
| | CGTGAAGCCCTTGAAAAATCTGAAACTTCATCAGCTTGGAACAATA | |
| | AACAGAATTTAAGTCAAGAAATCAAGTATGCATTAAAAACTTCATG | |
| | GCACGCTTCTGTACCACGTGTTGAAGCAAAACGTTATTGTCAAGTTT | |
| | ATCGTCCTGATTACGCTCGTATTGCTAAGTGTGTATACAAATTACCA | |
| | TACGTTAACAACGAAAAATTCTTAGAATTAGGTAAATTAGATTTTAA | |
| | CATCATTCAATCAATTCATCAAGAAGAAATGAAAAATGTGACAAGT | |
| | TGGTTTCGTGATTCTGGCTTACCATTATTTACTTTCGCTCGCGAACGT | |
| | CCTTTAGAATTTTACTTCTTAGTTGCTGCTGGTACTTATGAACCTCAA | |
| | TATGCTAAATGTCGTTTCTTATTCACAAAAGTAGCTTGTCTTCAAAC | |
| | AGTATTAGACGATATGTACGATACTTACGGTACTTTAGACGAATTAA | |
| | AACTTTTTACCGAGGCTGTGCGTCGTTGGGATTTATCTTTTACAGAA | |
| | AATTTACCTGACTATATGAAATTATGTTATCAAATCTATTATGACAT | |
| | CGTTCATGAAGTGGCTTGGGAAGCTGAAAAAGAACAAGGTAGAGAA | |
| | TTAGTGTCATTCTTCCGTAAAGGCTGGGAAGACTACTTATTAGGTTA | |
| | CTATGAAGAAGCAGAATGGTTAGCAGCAGAATACGTTCCAACATTA | |
| | GATGAATACATTAAAAACGGTATTACATCAATCGGCCAACGTATCTT | |
| | ATTACTTTCAGGTGTGTTAATTATGGATGGCCAACTTTTATCACAAG | |
| | AAGCATTAGAAAAAGTTGATTACCCTGGTCGTCGTGTTTTAACTGAG | |
| | TTAAACTCACTTATTAGCCGTTTAGCTGACGACACTAAAACTTATAA | |
| | AGCAGAAAAAGCTCGTGGAGAATTAGCCTCATCAATTGAATGCTAC | |
| | ATGAAAGATCATCCTGAATGTACAGAAGAAGAAGCCTTAGACCACA | |
| | TTTATTCTATTCTTGAACCAGCCGTAAAAGAATTAACTCGTGAATTT | |
| | CTTAAACCAGACGACGTTCCATTTGCTTGTAAAAAGATGTTATTCGA | |
| | AGAAACTCGTGTTACAATGGTGATCTTTAAAGATGGTGATGGTTTTG | |
| | GTGTATCTAAGTTAGAAGTTAAAGATCACATCAAAGAATGCTTAATT | |
| | GAACCATTACCATTAGGTACCGGTGAAAACTTATACTTTCAAGGCTC | |
| | AGGTGGCGGTGGAAGTGATTACAAAGATGATGATGATAAAGGAACC | |
| | GGTTAATCTAGACTCGAG | |
| 138 | CATATGGTACCAACTATGATGAATATGAATTTTAAGTACTGTCACAA | Diapophytoene |
| | GATTATGAAGAAACATTCAAAATCATTCAGTTATGCTTTTGACTTAT | (S. aureus) |
| | TACCAGAAGACCAACGTAAAGCTGTTTGGGCAATTTACGCCGTGTGC | |
| | CGCAAAATTGATGATTCTATTGATGTATATGGTGATATTCAATTCTT | |
| | AAATCAGATTAAAGAAGACATACAAAGTATTGAAAAATATCCATAC | |
| | GAACATCATCATTTTCAATCTGACAGACGTATTATGATGGCCTTACA | |
| | GCATGTTGCTCAGCATAAAAACATTGCATTTCAATCATTCTACAATT | |
| | TAATTGACACAGTATATAAAGATCAACACTTTACAATGTTTGAAACA | |
| | GATGCTGAACTTTTTGGTTATTGTTACGGTGTAGCTGGTACTGTGGG | |
| | TGAAGTTTTAACTCCTATATTATCTGATCACGAAACACATCAAACTT | |
| | ATGACGTTGCCCGTCGTTTAGGAGAGTCATTACAGTTAATCAATATT | |
| | CTTAGAGATGTAGGTGAAGACTTTGACAACGAACGTATTTACTTCTC | |
| | TAAACAACGTTTAAAACAATACGAAGTAGATATTGCAGAAGTGTAC | |
| | CAAAATGGTGTAAACAATCACTATATTGATTTATGGGAATATTACGC | |
| | TGCAATTGCTGAAAAGGATTTTCAAGATGTTATGGACCAAATTAAAG | |
| | TTTTCTCTATTGAAGCTCAGCCAATTATTGAGTTAGCTGCACGTATTT | |
| | ATATCGAAATTTTAGATGAAGTACGTCAAGCTAACTACACATTACAT | |
| | GAACGTGTTTTTGTAGATAAACGTAAAAAGGCTAAACTTTTTCACGA | |
| | AAATAAAGGTACCGGTGAAAACTTATACTTTCAAGGCTCAGGTGGC | |
| | GGTGGAAGTGATTACAAAGATGATGATGATAAAGGAACCGGTTAAT | |
| | CTAGACTCGAG | |
| 139 | CATATGGTACCAAAAATTGCTGTTATTGGTGCTGGTGTTACCGGATT | Diapophytoene |
| | AGCTGCTGCTGCTCGTATTGCTAGCCAAGGTCATGAAGTTACAATCT | desaturase |
| | TCGAAAAAAACAATAATGTAGGTGGTCGTATGAATCAATTAAAAAA | (S. aureus) |
| | AGATGGTTTTACATTCGATATGGGACCTACAATTGTTATGATGCCAG | |
| | ATGTATATAAAGATGTATTTACTGCTTGCGGTAAAAACTATGAAGAT | |
| | TATATAGAGTTACGTCAACTTCGTTACATTTATGACGTATATTTCGAT | |
| | CACGATGATCGTATTACTGTTCCAACTGATTTAGCTGAATTACAACA | |
| | AATGTTAGAATCAATTGAACCTGGTAGTACACACGGATTTATGTCAT | |
| | TTTTAACAGATGTGTACAAGAAATATGAAATCGCTCGCAGATATTTC | |
| | TTAGAACGTACTTACCGTAAACCATCAGACTTCTACAATATGACCTC | |
| | TTTAGTACAAGGTGCTAAACTTAAAACTTTAAATCACGCTGATCAAC | |
| | TTATCGAACACTACATTGATAACGAAAAGATTCAAAAACTTTTAGCA | |
| | TTCCAAACTCTTTATATCGGCATTGATCCAAAGCGTGGTCCTAGTTT | |
| | ATATAGTATTATTCCTATGATTGAAATGATGTTCGGTGTACATTTTAT | |
| | CAAAGGTGGTATGTATGGTATGGCTCAAGGATTAGCTCAACTTAACA | |
| | AAGATTTAGGTGTTAATATTGAATTAAATGCTGAAATTGAACAAATC | |
| | ATTATCGATCCTAAATTCAAACGCGCAGATGCAATTAAAGTTAATGG | |
| | TGACATTCGCAAATTTGATAAGATTTTATGTACTGCTGACTTTCCTTC | |
| | AGTTGCCGAATCACTTATGCCAGATTTCGCACCTATCAAAAAGTACC | |
| | CTCCACATAAAATTGCAGATTTAGATTATTCTTGTTCAGCTTTTCTTA | |
| | TGTATATTGGTATTGACATCGACGTAACTGACCAAGTTCGTTTACAT | |
| | AACGTAATTTTTAGCGACGATTTTCGTGGAAATATTGAAGAAATTTT | |
| | CGAAGGTCGCTTAAGTTACGACCCATCAATCTATGTTTATGTACCAG | |
| | CTGTAGCCGATAAATCTTTAGCTCCTGAAGGTAAAACAGGCATTTAT | |
| | GTGTTAATGCCTACTCCTGAACTTAAAACAGGATCAGGTATTGACTG | |
| | GTCAGATGAGGCTTTAACTCAACAAATTAAAGAAATCATTTATCGTA | |
| | AATTAGCAACAATTGAAGTATTTGAAGACATTAAATCACACATTGTA | |
| | TCAGAAACAATTTTTACTCCTAATGACTTTGAACAAACCTATCACGC | |
| | TAAATTTGGTTCTGCTTTCGGTTTAATGCCCACCTTAGCACAATCTAA | |
| | TTATTACAGACCTCAAAATGTGTCACGTGATTATAAAGACTTATATT | |
| | TCGCAGGTGCATCAACACATCCAGGTGCTGGAGTTCCAATTGTATTA | |
| | ACAAGTGCCAAGATAACAGTAGACGAAATGATTAAAGATATTGAGC | |
| | GTGGTGTGGGTACCGGTGAAAACTTATACTTTCAAGGCTCAGGTGGC | |
| | GGTGGAAGTGATTACAAAGATGATGATGATAAAGGAACCGGTTAAT | |
| | CTAGACTCGAG | |
| 140 | CATATGGTACCAGCATTTGACTTCGATGGTTACATGCTTCGTAAAGC | GPPS-LSU |
| | TAAATCTGTAAATAAAGCTCTTGAAGCTGCAGTACAAATGAAAGAA | (M. spicata) |
| | CCATTAAAAATTCATGAAAGTATGCGTTATTCTTTATTAGCTGGTGG | |
| | TAAACGTGTACGTCCAATGTTATGTATTGCAGCTTGTGAATTAGTTG | |
| | GTGGTGACGAAAGTACTGCTATGCCTGCTGCTTGCGCTGTAGAAATG | |
| | ATTCATACTATGAGTTTAATGCATGATGATTTACCATGTATGGATAA | |
| | TGACGATTTACGTCGTGGTAAACCAACAAACCACATGGCATTTGGTG | |
| | AAAGTGTAGCAGTATTAGCAGGTGATGCATTATTATCTTTTGCTTTT | |
| | GAACATGTAGCAGCAGCAACAAAAGGTGCTCCTCCAGAACGTATTG | |
| | TTAGAGTTTTAGGTGAACTTGCAGTTTCTATTGGTTCAGAAGGTTTA | |
| | GTTGCTGGACAAGTAGTTGACGTTTGTTCTGAAGGTATGGCTGAGGT | |
| | TGGTTTAGATCATTTAGAATTTATTCATCACCACAAAACTGCTGCTTT | |
| | ATTACAAGGTTCTGTAGTATTAGGTGCAATATTAGGTGGTGGAAAAG | |
| | AAGAAGAGGTAGCAAAACTTCGTAAATTCGCTAACTGCATTGGTTTA | |
| | CTTTTCCAAGTAGTAGATGATATTCTTGATGTAACAAAATCATCTAA | |
| | AGAATTAGGTAAAACAGCAGGTAAAGATTTAGTTGCTGATAAAACT | |
| | ACTTATCCAAAATTAATCGGTGTTGAGAAAAGTAAAGAGTTCGCAG | |
| | ACCGTTTAAATCGTGAAGCTCAAGAACAACTTCTTCATTTTCATCCA | |
| | CATAGAGCAGCACCTTTAATCGCTTTAGCAAACTATATTGCTTATCG | |
| | TGATAATGGTACCGGTGAAAATTTATATTTTCAAGGTTCAGGTGGCG | |
| | GAGGTTCTGATTATAAAGATGATGATGATAAAGGAACCGGTTAATC | |
| | TAGACTCGAG | |
| 141 | CATATGGTACCAAGTCAACCTTACTGGGCAGCAATTGAGGCAGATA | GPPS-SSU |
| | TTGAACGTTACTTAAAAAAATCAATTACAATTCGTCCACCAGAAACT | (M. spicata) |
| | GTATTTGGTCCAATGCACCACTTAACTTTTGCTGCACCAGCTACAGC | |
| | TGCTAGTACTTTATGTTTAGCAGCATGTGAACTTGTAGGTGGTGATC | |
| | GTAGTCAAGCTATGGCTGCAGCAGCAGCAATCCATCTTGTTCATGCA | |
| | GCTGCTTATGTACATGAACATTTACCATTAACTGATGGTAGTCGTCC | |
| | AGTAAGTAAACCAGCTATCCAACATAAATATGGTCCAAATGTAGAA | |
| | TTACTTACAGGTGACGGTATTGTACCATTTGGTTTTGAATTATTAGCA | |
| | GGTTCTGTTGATCCAGCACGTACAGATGATCCAGACCGTATTTTACG | |
| | TGTAATAATTGAAATAAGTCGTGCTGGTGGTCCAGAAGGTATGATTA | |
| | GTGGTTTACATCGTGAAGAAGAGATTGTAGATGGTAATACTTCTCTT | |
| | GATTTTATTGAATACGTTTGCAAAAAAAAATATGGTGAAATGCACGC | |
| | ATGTGGTGCTGCATGCGGTGCAATTTTAGGTGGTGCAGCTGAAGAA | |
| | GAAATTCAAAAACTTCGTAACTTCGGATTATATCAAGGAACTTTACG | |
| | TGGTATGATGGAGATGAAAAACTCACACCAACTTATTGACGAAAAT | |
| | ATCATTGGCAAACTTAAAGAATTAGCTTTAGAAGAATTAGGTGGATT | |
| | TCATGGTAAAAATGCTGAATTAATGTCTAGTTTAGTAGCAGAACCAT | |
| | CATTATATGCTGCTGGTACCGGTGAAAATTTATACTTTCAAGGTTCT | |
| | GGTGGTGGTGGCAGTGATTATAAAGACGATGATGACAAAGGAACCG | |
| | GTTAATCTAGACTCGAG | |
| 142 | CATATGGTACCACTTTTATCTAACAAATTAAGAGAGATGGTTTTAGC | GPPS (A. thalania) |
| | AGAAGTTCCTAAATTAGCATCTGCTGCTGAATATTTCTTTAAACGTG | |
| | GTGTTCAGGGTAAACAATTCCGTTCAACAATTTTATTATTAATGGCA | |
| | ACAGCTCTTGACGTTCGTGTTCCAGAAGCATTAATTGGTGAATCTAC | |
| | TGATATTGTAACATCTGAATTACGTGTACGTCAACGTGGCATTGCTG | |
| | AAATTACAGAAATGATTCATGTAGCATCACTTCTTCACGATGACGTT | |
| | CTTGACGATGCTGATACTCGTCGTGGTGTTGGTAGTCTTAATGTTGT | |
| | AATGGGAAACAAAATGTCAGTTTTAGCAGGTGACTTCTTACTTTCTC | |
| | GTGCTTGTGGTGCTCTTGCAGCTCTTAAAAACACAGAAGTTGTAGCA | |
| | TTATTAGCTACAGCAGTAGAACACTTAGTTACTGGTGAGACAATGGA | |
| | AATAACTTCATCAACTGAACAACGTTATTCTATGGATTACTACATGC | |
| | AGAAAACTTATTACAAAACTGCTTCATTAATTTCAAATTCATGTAAA | |
| | GCAGTTGCTGTATTAACAGGTCAAACAGCTGAAGTTGCAGTATTAGC | |
| | TTTTGAATATGGTCGTAATTTAGGTTTAGCTTTCCAGTTAATTGACGA | |
| | CATTTTAGATTTCACAGGCACATCTGCTAGTTTAGGAAAAGGTTCTT | |
| | TATCAGATATACGTCATGGTGTTATTACTGCTCCTATCTTATTTGCAA | |
| | TGGAAGAATTTCCTCAATTAAGAGAAGTAGTAGATCAAGTAGAAAA | |
| | AGATCCAAGAAATGTAGACATAGCTTTAGAATATTTAGGTAAAAGT | |
| | AAAGGTATTCAACGTGCTCGTGAATTAGCAATGGAACACGCAAATT | |
| | TAGCTGCTGCAGCTATTGGTTCTTTACCTGAAACAGATAACGAAGAT | |
| | GTTAAACGTTCACGTCGTGCTTTAATTGATTTAACACACAGAGTAAT | |
| | TACACGTAACAAAGGTACCGGTGAGAATTTATACTTTCAAGGTAGTG | |
| | GTGGAGGAGGTAGTGACTATAAAGATGATGACGATAAAGGAACCGG | |
| | TTAATCTAGACTCGAG | |
| 143 | CATATGGTACCAGTAGTTTCTGAACGTTTAAGACATTCTGTAACAAC | GPPS (C. reinhardtii) |
| | TGGTATTCCAGCATTAAAAACAGCAGCTGAATATTTCTTTCGTCGTG | |
| | GTATCGAAGGAAAACGTTTAAGACCTACATTAGCATTATTAATGAGT | |
| | AGTGCTTTATCACCAGCTGCTCCATCACCAGAGTATTTACAAGTTGA | |
| | TACAAGACCTGCTGCAGAACACCCTCATGAAATGCGTCGTCGTCAAC | |
| | AACGTTTAGCTGAAATTGCAGAATTAATCCATGTAGCTTCATTACTT | |
| | CACGATGATGTTATTGATGACGCACAAACACGTCGTGGTGTTTTAAG | |
| | TTTAAATACATCTGTTGGTAATAAAACAGCTATCTTAGCAGGTGATT | |
| | TCTTATTAGCTCGTGCATCTGTAACATTAGCTAGTTTAAGAAACTCT | |
| | GAAATTGTAGAATTAATGTCACAGGTTTTAGAACACTTAGTATCTGG | |
| | TGAAATTATGCAAATGACTGCTACTTCAGAACAACTTTTAGATTTAG | |
| | AACATTATTTAGCAAAAACATATTGTAAAACTGCTTCATTAATGGCT | |
| | AATAGTTCTCGTTCTGTTGCAGTTCTTGCAGGTGCAGCTCCTGAAGTT | |
| | TGTGATATGGCATGGTCATACGGTCGTCATTTAGGTATTGCTTTCCA | |
| | AGTAGTTGACGATTTATTAGATTTAACAGGTTCATCTTCTGTTTTAGG | |
| | TAAACCTGCTTTAAACGATATGCGTTCTGGTTTAGCAACAGCACCAG | |
| | TATTATTCGCTGCACAAGAAGAACCTGCATTACAGGCTCTTATATTA | |
| | CGTCGTTTTAAACACGACGGTGACGTAACAAAAGCAATGTCATTAAT | |
| | TGAACGTACACAAGGCTTACGTCGTGCTGAAGAACTTGCAGCACAA | |
| | CACGCAAAAGCTGCTGCTGATATGATTCGTTGCTTACCTACAGCTCA | |
| | ATCAGACCATGCAGAAATTGCTCGTGAAGCATTAATTCAAATTACAC | |
| | ATCGTGTTTTAACACGTAAAAAAGGTACCGGTGAAAACTTATACTTT | |
| | CAAGGTTCTGGTGGTGGTGGATCAGATTATAAAGATGATGATGACA | |
| | AAGGAACCGGTTAATCTAGACTCGAG | |
| 144 | CATATGGTACCAGATTTTCCACAACAATTAGAAGCATGTGTTAAACA | FPP (E. coli) |
| | AGCAAATCAAGCATTATCACGTTTCATCGCACCACTTCCATTCCAAA | |
| | ATACTCCTGTTGTTGAAACAATGCAATATGGTGCATTATTAGGAGGT | |
| | AAAAGATTAAGACCATTTCTTGTATATGCAACAGGTCACATGTTTGG | |
| | AGTATCTACTAACACATTAGATGCTCCAGCTGCTGCAGTTGAATGTA | |
| | TTCATGCATATAGTTTAATTCATGATGATTTACCTGCAATGGATGAT | |
| | GATGACTTAAGAAGAGGTTTACCTACATGTCATGTTAAATTTGGTGA | |
| | AGCTAATGCTATTTTAGCTGGCGATGCACTTCAAACTCTTGCATTCA | |
| | GTATTTTATCAGATGCTGATATGCCAGAAGTTTCAGATCGTGATCGT | |
| | ATTTCTATGATATCTGAATTAGCTTCTGCTAGTGGTATTGCTGGTATG | |
| | TGCGGTGGCCAAGCTCTTGATTTAGACGCAGAAGGAAAACACGTTC | |
| | CTTTAGATGCTTTAGAGCGTATACATCGTCACAAAACAGGAGCTTTA | |
| | ATTAGAGCTGCTGTTCGTCTTGGTGCTTTATCAGCTGGAGACAAAGG | |
| | TCGTCGTGCTTTACCAGTTTTAGACAAATACGCTGAAAGTATTGGTT | |
| | TAGCTTTTCAAGTTCAGGATGATATCTTAGATGTTGTAGGTGATACT | |
| | GCTACTTTAGGTAAACGTCAAGGTGCTGATCAACAGTTAGGCAAATC | |
| | TACATACCCAGCACTTTTAGGTTTAGAACAAGCTCGTAAAAAAGCA | |
| | AGAGACTTAATTGACGATGCTCGTCAAAGTCTTAAACAATTAGCAG | |
| | AACAATCACTTGATACAAGTGCTTTAGAAGCATTAGCAGATTACATT | |
| | ATTCAACGTAATAAAGGTACCGGTGAAAATTTATATTTTCAAGGTTC | |
| | TGGTGGTGGAGGTTCAGACTATAAAGATGACGATGATAAAGGAACC | |
| | GGTTAATCTAGACTCGAG | |
| 145 | CATATGGTACCAAGTGTTAGTTGTTGTTGTAGAAATTTAGGAAAAAC | FPP (A. thalania) |
| | TATCAAAAAAGCTATTCCAAGTCACCACTTACATTTACGTTCTTTAG | |
| | GTGGTAGTTTATATAGAAGACGTATTCAATCATCTTCAATGGAAACA | |
| | GACTTAAAATCTACATTCTTAAATGTTTATTCAGTTCTTAAATCAGAT | |
| | TTATTACACGACCCATCATTTGAATTTACAAATGAAAGTCGTTTATG | |
| | GGTAGATAGAATGCTTGATTATAATGTTCGTGGCGGTAAACTTAATC | |
| | GTGGTCTTTCTGTAGTAGACTCTTTCAAATTACTTAAACAAGGTAAT | |
| | GATTTAACTGAACAAGAAGTTTTCTTATCTTGTGCATTAGGTTGGTG | |
| | TATTGAGTGGTTACAGGCTTACTTTTTAGTTCTTGATGATATTATGGA | |
| | TAATTCAGTTACACGTCGTGGTCAACCTTGTTGGTTTCGTGTACCAC | |
| | AAGTTGGTATGGTAGCTATTAATGATGGCATTCTTCTTCGTAACCAT | |
| | ATTCATCGTATTCTTAAAAAACACTTCCGTGATAAACCATATTATGT | |
| | AGATTTAGTTGACCTTTTCAATGAAGTAGAGTTACAAACTGCATGTG | |
| | GACAAATGATTGATTTAATCACAACATTTGAAGGTGAAAAAGACTT | |
| | AGCTAAATATAGTTTATCAATTCACCGTCGTATTGTTCAATACAAAA | |
| | CTGCATATTACTCATTCTATTTACCAGTTGCATGTGCTCTTTTAATGG | |
| | CTGGCGAAAATTTAGAAAACCACATTGATGTTAAAAATGTATTAGTA | |
| | GATATGGGTATTTACTTTCAAGTTCAGGATGATTATTTAGACTGTTTT | |
| | GCTGATCCTGAAACATTAGGTAAAATTGGCACTGATATTGAGGACTT | |
| | TAAATGTTCTTGGTTAGTTGTAAAAGCATTAGAACGTTGTAGTGAAG | |
| | AACAAACAAAAATTCTTTACGAAAACTATGGCAAACCTGATCCATCT | |
| | AATGTTGCTAAAGTAAAAGATTTATACAAAGAATTAGATTTAGAAG | |
| | GCGTTTTCATGGAATATGAATCTAAATCATACGAGAAATTAACTGGT | |
| | GCTATCGAAGGTCACCAATCTAAAGCAATTCAAGCTGTTCTTAAATC | |
| | TTTCTTAGCAAAAATCTATAAACGTCAAAAAGGTACCGGTGAAAAC | |
| | TTATACTTTCAAGGTAGTGGTGGCGGTGGTAGTGATTATAAAGATGA | |
| | TGATGATAAAGGAACCGGTTAATCTAGACTCGAG | |
| 146 | CATATGGTACCAGCTGATCTTAAATCAACATTCTTAGATGTTTATTC | FPP (A. thalania) |
| | AGTATTAAAAAGTGATTTATTACAAGATCCATCTTTTGAATTTACAC | |
| | ACGAAAGTCGTCAATGGTTAGAACGTATGTTAGATTATAATGTTCGT | |
| | GGAGGCAAATTAAACAGAGGTTTAAGTGTAGTAGACAGTTACAAAC | |
| | TTTTAAAACAAGGTCAAGACTTAACAGAAAAAGAAACATTTTTATCT | |
| | TGTGCTTTAGGTTGGTGTATTGAATGGTTACAAGCATACTTCTTAGTT | |
| | TTAGACGATATTATGGATAATTCTGTAACTAGACGTGGTCAACCATG | |
| | TTGGTTTCGTAAACCAAAAGTAGGTATGATTGCTATTAATGATGGAA | |
| | TACTTCTTCGTAACCACATTCATCGTATTCTTAAAAAACACTTTCGTG | |
| | AAATGCCTTATTATGTAGACCTTGTAGACTTATTTAACGAAGTAGAA | |
| | TTTCAAACAGCTTGTGGTCAAATGATTGACTTAATTACAACATTTGA | |
| | TGGTGAAAAAGACCTTTCAAAATATTCACTTCAGATTCACCGTCGTA | |
| | TTGTTGAGTACAAAACAGCATACTACTCTTTCTATTTACCTGTAGCAT | |
| | GTGCTTTACTTATGGCAGGTGAAAATTTAGAAAATCACACAGATGTT | |
| | AAAACTGTATTAGTTGATATGGGTATCTATTTCCAAGTTCAAGATGA | |
| | TTATTTAGATTGCTTCGCTGATCCAGAAACATTAGGTAAAATTGGTA | |
| | CAGATATTGAAGACTTTAAATGTAGTTGGTTAGTAGTAAAAGCATTA | |
| | GAACGTTGTAGTGAAGAACAAACAAAAATTCTTTACGAAAATTATG | |
| | GAAAAGCTGAACCTTCAAATGTAGCTAAAGTTAAAGCATTATACAA | |
| | AGAATTAGATTTAGAGGGTGCATTTATGGAATATGAAAAAGAATCA | |
| | TACGAGAAACTTACAAAACTTATTGAAGCACATCAATCAAAAGCTA | |
| | TTCAAGCAGTTCTTAAATCTTTCTTAGCTAAAATTTATAAACGTCAA | |
| | AAAGGTACCGGTGAAAACTTATACTTTCAAGGCTCTGGAGGTGGTG | |
| | GTTCAGACTATAAAGATGATGATGATAAAGGAACCGGTTAATCTAG | |
| | ACTCGAG | |
| 147 | CATATGGTACCAAGTGGCGAACCTACTCCAAAAAAAATGAAAGCAA | FPP (C. reinhardtii) |
| | CATACGTTCACGACCGTGAAAACTTTACAAAAGTATACGAAACTCTT | |
| | CGTGACGAATTACTTAACGATGATTGTCTTAGTCCAGCTGGTTCACC | |
| | TCAGGCTCAAGCTGCTCAAGAGTGGTTTAAAGAAGTTAATGATTATA | |
| | ATGTTCCTGGTGGAAAACTTAACCGTGGTATGGCTGTATATGACGTT | |
| | TTAGCTTCAGTTAAAGGTCCAGATGGTTTAAGTGAAGACGAAGTATT | |
| | TAAAGCTAACGCTCTTGGTTGGTGTATTGAGTGGTTACAAGCATTTT | |
| | TCTTAGTTGCTGATGATATAATGGATGGTTCAATTACACGTCGTGGC | |
| | CAACCTTGTTGGTACAAACAACCTAAAGTTGGTATGATTGCTTGTAA | |
| | TGATTACATCTTATTAGAATGCTGTATTTACTCAATTCTTAAAAGAC | |
| | ATTTTAGAGGTCACGCTGCATACGCTCAACTTATGGACCTTTTCCAT | |
| | GAAACTACATTCCAGACTTCACACGGTCAATTATTAGATTTAACAAC | |
| | AGCACCTATCGGTTCTGTAGACTTATCAAAATATACAGAAGATAATT | |
| | ACCTTCGTATTGTAACATATAAAACTGCATACTATTCTTTTTATTTAC | |
| | CTGTAGCATGTGGTATGGTATTAGCTGGCATTACAGATCCAGCTGCT | |
| | TTTGATCTTGCAAAAAATATTTGTGTTGAAATGGGTCAATATTTCCA | |
| | GATTCAAGACGATTATTTAGATTGCTATGGTGACCCTGAGGTTATTG | |
| | GTAAAATCGGTACAGACATAGAAGACAACAAATGTAGTTGGTTAGT | |
| | TTGCACAGCTCTTAAAATCGCAACAGAAGAACAAAAAGAGGTTATA | |
| | AAAGCTAATTATGGTCACAAAGAGGCTGAATCAGTAGCAGCAATTA | |
| | AAGCATTATACGTTGAATTAGGTATTGAACAACGTTTTAAAGACTAT | |
| | GAAGCTGCATCATACGCAAAATTAGAAGGTACAATTAGTGAACAAA | |
| | CTTTATTACCTAAAGCAGTATTTACTTCTTTATTAGCTAAAATCTATA | |
| | AAAGAAAAAAAGGTACCGGTGAGAACTTATACTTTCAAGGTAGTGG | |
| | AGGTGGTGGTTCAGACTATAAAGATGATGATGATAAAGGAACCGGT | |
| | TAATCTAGACTCGAG | |
| 148 | CATATGGTACCACAAACTGAACATGTTATCTTATTAAACGCTCAAGG | IPP |
| | TGTTCCTACAGGTACATTAGAAAAATATGCTGCACACACTGCTGATA | isomerase |
| | CTCGTTTACACTTAGCTTTCTCATCTTGGTTATTCAATGCTAAAGGTC | (E. coli) |
| | AACTTTTAGTTACAAGACGTGCATTAAGTAAAAAAGCATGGCCTGGT | |
| | GTTTGGACTAACTCAGTTTGTGGTCATCCACAATTAGGTGAAAGTAA | |
| | TGAAGATGCAGTTATACGTCGTTGCAGATATGAATTAGGTGTTGAAA | |
| | TAACTCCACCAGAATCAATTTATCCAGATTTCCGTTATCGTGCAACT | |
| | GATCCTAGTGGTATCGTTGAAAACGAAGTATGTCCTGTTTTTGCTGC | |
| | ACGTACAACAAGTGCATTACAAATTAATGATGATGAAGTAATGGAT | |
| | TATCAATGGTGTGACTTAGCTGATGTTTTACATGGTATTGATGCAAC | |
| | ACCATGGGCATTTTCACCATGGATGGTAATGCAAGCAACAAATCGT | |
| | GAAGCACGTAAAAGATTAAGTGCTTTTACACAGTTAAAAGGTACCG | |
| | GTGAAAACTTATACTTTCAAGGTAGTGGAGGTGGTGGTTCTGACTAT | |
| | AAAGATGACGATGATAAAGGAACCGGTTAATCTAGACTCGAG | |
| 149 | CATATGGTACCACTTCGTAGTTTATTAAGAGGTTTAACACACATTCC | IPP |
| | TCGTGTTAATAGTGCTCAGCAACCTTCTTGCGCTCACGCTCGTCTTCA | isomerase |
| | ATTTAAACTTCGTTCTATGCAATTATTAGCAGAAAACCGTACAGATC | (H. pluvalis) |
| | ACATGCGTGGTGCTTCTACATGGGCAGGTGGTCAGTCTCAAGATGAA | |
| | TTAATGCTTAAAGATGAATGTATCTTAGTAGATGCTGATGATAACAT | |
| | TACTGGTCACGCTTCTAAATTAGAATGTCACAAATTTCTTCCACATC | |
| | AACCAGCTGGATTATTACACCGTGCTTTTTCTGTATTTCTTTTCGACG | |
| | ATCAAGGTCGTTTACTTTTACAACAACGTGCTCGTAGTAAAATTACA | |
| | TTTCCATCTGTATGGGCTAATACATGTTGTAGTCATCCATTACATGGT | |
| | CAAACACCAGATGAAGTAGATCAACAATCACAAGTAGCAGACGGAA | |
| | CTGTACCAGGTGCAAAAGCTGCTGCAATCAGAAAATTAGAACATGA | |
| | ATTAGGTATTCCAGCTCACCAATTACCAGCATCAGCTTTTCGTTTCTT | |
| | AACACGTCTTCACTATTGTGCAGCTGACGTTCAACCTGCAGCAACAC | |
| | AATCTGCATTATGGGGTGAACACGAAATGGATTACATTTTATTCATT | |
| | AGAGCTAATGTTACACTTGCTCCTAATCCTGACGAAGTAGATGAGGT | |
| | ACGTTATGTAACTCAAGAAGAATTAAGACAAATGATGCAACCAGAT | |
| | AATGGTTTACAATGGTCACCATGGTTCCGTATTATTGCAGCAAGATT | |
| | TTTAGAACGTTGGTGGGCTGATTTAGATGCTGCATTAAATACAGATA | |
| | AACATGAAGACTGGGGAACAGTTCATCACATTAACGAAGCTGGTAC | |
| | CGGTGAAAACTTATACTTTCAAGGATCAGGAGGCGGTGGAAGTGAT | |
| | TATAAAGATGATGATGATAAAGGAACCGGTTAATCTAGACTCGAG | |
| 150 | CATATGGTACCAAGAAGATCAGGCAATTATAACCCAACAGCATGGG | Limonene |
| | ACTTCAATTATATCCAATCATTAGACAATCAATACAAAAAAGAACGT | (L. angustifolia) |
| | TACTCTACTCGTCACGCTGAATTAACAGTTCAAGTTAAAAAATTATT | |
| | AGAAGAAGAAATGGAAGCTGTTCAAAAACTTGAACTTATAGAGGAT | |
| | CTTAAAAACTTAGGCATTTCTTACCCATTTAAAGATAATATTCAACA | |
| | AATCTTAAATCAAATTTACAATGAACACAAATGTTGTCACAACTCAG | |
| | AAGTTGAAGAAAAAGACCTTTATTTCACTGCTTTACGTTTTAGATTA | |
| | TTACGTCAACAAGGTTTTGAAGTAAGTCAAGAAGTATTTGATCACTT | |
| | TAAAAACGAAAAAGGTACAGATTTTAAACCTAATTTAGCAGATGAT | |
| | ACTAAAGGATTATTACAATTATATGAAGCATCATTCTTATTACGTGA | |
| | AGCAGAAGACACATTAGAACTTGCTCGTCAATTCTCTACTAAACTTT | |
| | TACAAAAAAAAGTTGATGAAAACGGTGACGATAAAATTGAAGATAA | |
| | CTTATTACTTTGGATTAGACGTAGTTTAGAATTACCATTACATTGGC | |
| | GTGTACAAAGATTAGAAGCTCGTGGCTTTTTAGATGCTTACGTTCGT | |
| | AGACCTGATATGAATCCTATTGTATTTGAATTAGCAAAATTAGACTT | |
| | TAACATTACTCAAGCAACACAACAAGAAGAACTTAAAGATTTATCA | |
| | AGATGGTGGAATAGTACTGGCTTAGCTGAAAAACTTCCTTTTGCTCG | |
| | TGATCGTGTAGTTGAATCATATTTCTGGGCTATGGGTACTTTTGAAC | |
| | CACATCAATACGGATACCAACGTGAATTAGTTGCTAAAATCATTGCA | |
| | CTTGCTACAGTTGTAGACGATGTTTACGATGTATATGGTACTTTAGA | |
| | GGAATTAGAACTTTTTACTGATGCTATTCGTCGTTGGGACCGTGAAT | |
| | CTATTGACCAATTACCATATTACATGCAATTATGTTTTCTTACTGTAA | |
| | ACAACTTTGTTTTTGAGTTAGCTCACGACGTATTAAAAGATAAATCA | |
| | TTCAATTGTTTACCTCATTTACAAAGATCATGGTTAGATTTAGCTGA | |
| | AGCATACCTTGTAGAAGCAAAATGGTATCATAGTCGTTATACACCTT | |
| | CTTTAGAAGAATATCTTAATATTGCTCGTGTTTCAGTAACATGTCCA | |
| | ACTATTGTTTCTCAAATGTATTTTGCATTACCAATTCCAATCGAAAA | |
| | ACCTGTAATTGAGATCATGTACAAATATCACGATATCTTATACTTAT | |
| | CAGGTATGTTATTACGTTTACCAGATGACTTAGGAACAGCATCATTC | |
| | GAACTTAAACGTGGTGATGTACAAAAAGCAGTTCAATGTTATATGA | |
| | AAGAACGTAATGTTCCTGAAAATGAAGCTCGTGAACATGTTAAATTC | |
| | TTAATTCGTGAGGCTTCTAAACAAATTAATACAGCAATGGCAACAG | |
| | ACTGTCCATTTACAGAAGATTTTGCAGTTGCAGCAGCAAACTTAGGT | |
| | CGTGTAGCAAATTTTGTATATGTTGATGGTGATGGTTTTGGAGTACA | |
| | ACACAGTAAAATCTATGAGCAAATTGGTACACTTATGTTTGAACCAT | |
| | ATCCAGGTACCGGTGAAAACTTATACTTTCAAGGTAGTGGTGGTGGA | |
| | GGTTCTGATTACAAAGACGATGATGATAAAGGAACCGGTTAATCTA | |
| | GACTCGAG | |
| 151 | CATATGGTACCAAGAAGAAGTGGAAACTATAAACCTACAATGTGGG | Monoterpene |
| | ATTTTCAATTTATTCAAAGTGTAAATAATCTTTACGCTGGTGATAAA | (S. lycopersicum) |
| | TACATGGAACGTTTCGATGAAGTAAAAAAAGAAATGAAAAAAAACT | |
| | TAATGATGATGGTTGAGGGTTTAATAGAGGAATTAGATGTTAAATTA | |
| | GAATTAATAGATAATTTAGAAAGATTAGGTGTTAGTTATCATTTCAA | |
| | AAATGAAATAATGCAAATCCTTAAATCTGTACACCAGCAAATCACTT | |
| | GTCGTGATAATTCATTATACTCTACTGCATTAAAATTTCGTTTATTAC | |
| | GTCAACACGGATTCCACATTAGTCAAGACATCTTTAACGATTTTAAA | |
| | GATATGAATGGCAATGTTAAACAAAGTATCTGTAACGATACTAAAG | |
| | GTTTATTAGAACTTTATGAAGCATCTTTCTTATCTACTGAATGTGAAA | |
| | CAACACTTAAAAACTTCACTGAAGCACACTTAAAAAATTATGTTTAT | |
| | ATTAACCACTCATGTGGAGATCAATACAATAACATAATGATGGAATT | |
| | AGTTGTTCACGCTTTAGAATTACCACGTCACTGGATGATGCCTCGTT | |
| | TAGAGACACGTTGGTATATATCAATTTATGAACGTATGCCTAATGCT | |
| | AATCCACTTTTACTTGAACTTGCTAAATTAGACTTCAATATTGTTCAA | |
| | GCTACACACCAACAAGACTTAAAATCATTATCACGTTGGTGGAAAA | |
| | ACATGTGTTTAGCTGAAAAATTATCATTTTCTCGTAACCGTTTAGTA | |
| | GAAAATCTTTTCTGGGCAGTTGGAACTAATTTTGAACCACAACACAG | |
| | TTATTTCCGTCGTTTAATCACTAAAATCATTGTTTTTGTTGGTATTAT | |
| | TGATGATATTTATGATGTTTACGGCAAACTTGATGAGTTAGAATTAT | |
| | TCACTTTAGCTGTACAACGTTGGGATACAAAAGCAATGGAAGACTT | |
| | ACCATATTACATGCAAGTTTGTTATTTAGCTTTAATTAATACAACAA | |
| | ATGATGTTGCTTATGAAGTTCTTCGTAAACATAACATTAATGTATTA | |
| | CCATACTTAACTAAATCTTGGACAGACTTATGTAAATCATATTTACA | |
| | AGAAGCTCGTTGGTACTACAATGGTTACAAACCTTCATTAGAGGAAT | |
| | ATATGGATAATGGTTGGATTAGTATAGCAGTTCCTATGGTATTAGCA | |
| | CATGCACTTTTCTTAGTTACAGATCCAATTACAAAAGAAGCATTAGA | |
| | ATCATTAACAAACTATCCTGATATTATTCGTTGCTCAGCTACAATATT | |
| | CCGTTTAAATGATGATCTTGGTACAAGTTCAGATGAATTAAAACGTG | |
| | GAGATGTACCAAAATCAATTCAATGCTATATGAACGAAAAAGGCGT | |
| | TTCAGAGGAAGAAGCTCGTGAACATATTCGTTTCTTAATCAAAGAAA | |
| | CATGGAAATTCATGAACACTGCACACCATAAAGAGAAAAGTTTATT | |
| | TTGTGAGACATTTGTAGAAATTGCAAAAAATATTGCAACAACAGCTC | |
| | ATTGTATGTACTTAAAAGGTGATTCTCACGGTATTCAAAACACTGAT | |
| | GTTAAAAACTCAATAAGTAATATACTTTTCCATCCAATTATTATCGG | |
| | TACCGGTGAAAACCTTTACTTTCAAGGTTCAGGTGGTGGCGGTTCAG | |
| | ACTATAAAGATGACGATGATAAAGGAACCGGTTAATCTAGACTCGAG | |
| 152 | CATATGGTACCAAGACGTAGTGGAAATTATGAGCCATCTGCATGGG | Terpinolene |
| | ACTTCAATTACTTACAATCTCTTAATAATTATCACCATAAAGAAGAA | (O. basilicum) |
| | CGTTACTTACGTCGTCAAGCTGATTTAATTGAAAAAGTAAAAATGAT | |
| | TCTTAAAGAAGAGAAAATGGAAGCATTACAGCAATTAGAACTTATA | |
| | GACGATCTTCGTAATTTAGGTCTTTCATATTGTTTTGATGATCAAATT | |
| | AATCATATTCTTACAACAATTTACAACCAACATTCTTGTTTCCATTAT | |
| | CACGAAGCTGCAACAAGTGAAGAAGCTAACTTATATTTCACAGCTTT | |
| | AGGTTTCCGTTTACTTCGTGAACACGGATTCAAAGTATCACAAGAAG | |
| | TATTTGACCGTTTCAAAAATGAAAAAGGTACAGATTTTCGTCCAGAT | |
| | TTAGTAGATGATACTCAAGGTTTATTACAACTTTATGAAGCATCTTT | |
| | CCTTCTTCGTGAAGGTGAAGACACTTTAGAATTTGCACGTCAATTTG | |
| | CTACTAAATTTCTTCAAAAAAAAGTTGAGGAGAAAATGATAGAAGA | |
| | GGAAAATCTTTTATCTTGGACTTTACATTCACTTGAATTACCATTACA | |
| | TTGGCGTATACAACGTTTAGAAGCTAAATGGTTTTTAGATGCTTATG | |
| | CTAGTCGTCCTGATATGAATCCAATAATCTTTGAATTAGCAAAATTA | |
| | GAATTTAACATTGCTCAGGCACTTCAACAAGAAGAACTTAAAGATTT | |
| | ATCAAGATGGTGGAACGATACTGGTATTGCTGAAAAATTACCTTTCG | |
| | CTCGTGATAGAATCGTTGAATCTCATTATTGGGCAATTGGTACTTTA | |
| | GAACCTTATCAATACCGTTATCAGCGTTCATTAATTGCAAAAATCAT | |
| | TGCTTTAACTACAGTTGTTGATGATGTATATGATGTTTACGGTACATT | |
| | AGACGAATTACAGTTATTTACTGATGCAATTCGTCGTTGGGACATTG | |
| | AAAGTATAAATCAATTACCTTCTTATATGCAATTATGTTATTTAGCTA | |
| | TTTATAATTTCGTATCAGAATTAGCTTATGATATTTTCAGAGATAAA | |
| | GGTTTTAATTCTTTACCATATTTACACAAAAGTTGGCTTGACTTAGTT | |
| | GAGGCTTACTTTCAAGAAGCAAAATGGTATCATTCTGGCTACACACC | |
| | ATCATTAGAACAATACTTAAATATCGCTCAAATTTCTGTAGCAAGTC | |
| | CAGCTATATTAAGTCAAATTTACTTTACTATGGCTGGTTCAATTGAT | |
| | AAACCAGTAATCGAATCAATGTACAAATATAGACACATTTTAAACTT | |
| | ATCTGGTATATTACTTAGATTACCAGATGACTTAGGTACTGCTAGTG | |
| | ATGAATTAGGTCGTGGTGATTTAGCAAAAGCAATGCAATGTTACATG | |
| | AAAGAGCGTAACGTTTCTGAAGAAGAAGCTCGTGATCATGTACGTTT | |
| | CTTAAATCGTGAGGTTTCAAAACAAATGAATCCTGCTCGTGCTGCTG | |
| | ATGATTGTCCATTCACTGATGATTTTGTAGTAGCTGCTGCTAATTTAG | |
| | GAAGAGTTGCAGATTTCATGTATGTTGAAGGCGATGGTTTAGGTTTA | |
| | CAATACCCAGCTATCCACCAACACATGGCAGAACTTTTATTTCACCC | |
| | TTACGCAGGTACCGGTGAAAACTTATACTTTCAAGGTTCAGGTGGTG | |
| | GAGGTTCTGACTATAAAGATGATGATGATAAAGGAACCGGTTAATC | |
| | TAGACTCGAG | |
| 153 | CATATGGTACCAAGAAGATCAGGAAATTATCAACCTAGTGCATGGG | Myrcene (O. basilicum) |
| | ATTTTAACTATATCCAATCTCTTAATAACAACCATTCTAAAGAAGAA | |
| | CGTCACTTAGAGCGTAAAGCAAAACTTATTGAAGAAGTAAAAATGT | |
| | TATTAGAGCAAGAAATGGCTGCTGTACAACAATTAGAGCTTATTGA | |
| | AGACCTTAAAAACTTAGGTTTATCTTACTTATTCCAAGATGAAATCA | |
| | AAATAATCCTTAATTCTATTTACAATCATCATAAATGTTTTCATAATA | |
| | ATCACGAACAATGTATTCACGTTAATAGTGACTTATACTTTGTTGCA | |
| | TTAGGCTTCCGTTTATTTCGTCAACATGGTTTCAAAGTTTCTCAAGAG | |
| | GTTTTTGACTGTTTTAAAAACGAAGAAGGATCAGACTTTAGTGCTAA | |
| | CTTAGCAGATGATACTAAAGGTTTACTTCAATTATACGAGGCTTCAT | |
| | ATTTAGTTACAGAAGATGAAGACACATTAGAAATGGCACGTCAATT | |
| | TTCAACTAAAATCTTACAAAAAAAAGTAGAAGAGAAAATGATTGAG | |
| | AAAGAGAACTTATTAAGTTGGACTTTACATAGTTTAGAATTACCACT | |
| | TCACTGGCGTATTCAACGTTTAGAAGCAAAATGGTTCCTTGATGCTT | |
| | ATGCTAGTCGTCCAGATATGAATCCAATTATTTTTGAATTAGCTAAA | |
| | TTAGAGTTTAACATTGCTCAAGCATTACAACAAGAAGAATTAAAAG | |
| | ATTTAAGTAGATGGTGGAATGATACAGGCATTGCTGAAAAATTACCT | |
| | TTTGCTCGTGATAGAATAGTAGAGAGTCATTACTGGGCAATTGGTAC | |
| | TTTAGAACCTTATCAATATAGATATCAACGTTCATTAATTGCTAAAA | |
| | TTATTGCTTTAACAACAGTTGTTGATGACGTTTACGACGTATATGGA | |
| | ACTTTAGATGAATTACAGTTATTTACAGACGCTATTCGTCGTTGGGA | |
| | TATTGAATCTATTAATCAATTACCAAGTTATATGCAATTATGCTATTT | |
| | AGCTATTTATAACTTTGTTTCTGAATTAGCATACGATATTTTTCGTGA | |
| | CAAAGGATTCAATTCTTTACCTTACCTTCATAAATCATGGTTAGATTT | |
| | AGTAGAAGCATACTTTGTTGAAGCTAAATGGTTTCATGATGGTTATA | |
| | CTCCAACTCTTGAAGAATATTTAAATAACTCAAAAATTACTATTATA | |
| | TGTCCTGCTATTGTTAGTGAAATCTACTTCGCATTCGCTAATTCAATT | |
| | GATAAAACAGAAGTTGAATCAATCTACAAATATCACGATATTTTATA | |
| | TTTATCAGGAATGCTTGCACGTTTACCAGACGACTTAGGTACTTCAT | |
| | CATTTGAAATGAAAAGAGGTGATGTTGCTAAAGCTATTCAATGTTAC | |
| | ATGAAAGAACATAATGCTTCAGAGGAAGAAGCTCGTGAACACATTC | |
| | GTTTCTTAATGCGTGAAGCATGGAAACACATGAATACTGCTGCAGCT | |
| | GCTGATGACTGTCCATTTGAATCTGATTTAGTAGTAGGTGCTGCATC | |
| | ATTAGGTAGAGTTGCAAACTTTGTATATGTTGAAGGTGACGGTTTTG | |
| | GTGTACAACATTCAAAAATACATCAACAAATGGCTGAATTACTTTTT | |
| | TATCCATATCAAGGTACCGGTGAAAACTTATACTTTCAAGGTAGTGG | |
| | AGGTGGTGGTAGTGACTATAAAGACGATGACGATAAAGGAACCGGT | |
| | TAATCTAGACTCGAG | |
| 154 | CATATGGTACCAAGAAGAAGTGCTAATTATCAAGCAAGTATTTGGG | Zingiberene |
| | ATGATAATTTCATTCAAAGTCTTGCATCTCCTTATGCAGGAGAAAAA | (O. basilicum) |
| | TATGCAGAAAAAGCAGAAAAACTTAAAACAGAAGTTAAAACTATGA | |
| | TTGATCAAACAAGAGATGAACTTAAACAATTAGAACTTATTGATAA | |
| | CTTACAACGTTTAGGTATATGTCATCACTTTCAAGACCTTACAAAAA | |
| | AAATTTTACAAAAAATTTATGGAGAAGAACGTAACGGAGATCACCA | |
| | ACATTACAAAGAAAAAGGCTTACATTTTACAGCATTACGTTTCCGTA | |
| | TTTTACGTCAGGACGGTTATCATGTTCCACAAGATGTATTTTCATCAT | |
| | TTATGAATAAAGCTGGTGACTTTGAAGAATCTTTAAGTAAAGACACA | |
| | AAAGGTTTAGTTAGTTTATATGAGGCTTCTTACTTATCAATGGAAGG | |
| | TGAAACTATTTTAGATATGGCAAAAGACTTTTCATCTCACCATTTAC | |
| | ATAAAATGGTTGAAGATGCTACTGACAAACGTGTAGCTAATCAAAT | |
| | TATCCATTCTCTTGAAATGCCACTTCACAGACGTGTTCAAAAACTTG | |
| | AAGCAATTTGGTTTATTCAATTCTACGAATGCGGCTCTGATGCTAAT | |
| | CCAACTTTAGTAGAATTAGCAAAATTAGATTTCAACATGGTTCAGGC | |
| | AACATACCAAGAAGAATTAAAACGTTTATCACGTTGGTATGAAGAA | |
| | ACAGGCTTACAAGAGAAACTTTCATTCGCTCGTCACCGTCTTGCTGA | |
| | AGCATTCTTATGGTCTATGGGTATTATTCCAGAAGGACACTTTGGTT | |
| | ATGGTCGTATGCACTTAATGAAAATTGGTGCTTACATTACATTACTT | |
| | GATGATATTTATGATGTTTATGGTACTTTAGAAGAACTTCAAGTATT | |
| | AACAGAAATTATTGAACGTTGGGATATTAACTTATTAGATCAATTAC | |
| | CTGAATACATGCAAATCTTCTTTTTATACATGTTTAATTCTACAAATG | |
| | AACTTGCTTATGAAATTTTACGTGATCAAGGTATCAATGTAATATCA | |
| | AACTTAAAAGGATTATGGGTAGAGTTATCTCAGTGTTACTTTAAAGA | |
| | AGCTACTTGGTTCCATAACGGTTACACACCAACAACTGAAGAATATC | |
| | TTAATGTTGCTTGTATTTCTGCTAGTGGTCCTGTTATTTTATTTTCAG | |
| | GTTACTTTACTACTACTAATCCTATTAATAAACACGAATTACAATCTT | |
| | TAGAACGTCACGCACATTCATTATCTATGATATTACGTTTAGCTGAT | |
| | GATTTAGGTACATCAAGTGATGAAATGAAACGTGGAGATGTACCAA | |
| | AAGCTATTCAATGTTTTATGAATGACACTGGTTGTTGTGAAGAAGAA | |
| | GCACGTCAACACGTAAAAAGATTAATAGATGCTGAATGGAAAAAAA | |
| | TGAACAAAGACATCTTAATGGAGAAACCATTTAAAAATTTTTGTCCA | |
| | ACTGCTATGAATTTAGGTCGTATTTCTATGAGTTTTTATGAACACGG | |
| | AGATGGTTATGGAGGTCCTCACTCTGATACAAAAAAAAAAATGGTA | |
| | TCTTTATTTGTACAACCAATGAATATTACTATTGGTACCGGTGAAAA | |
| | CCTTTATTTTCAAGGTTCTGGTGGTGGCGGTTCAGATTATAAAGATG | |
| | ATGACGACAAAGGAACCGGTTAATCTAGACTCGAG | |
| 155 | CATATGGTACCAAGACGTTCAGCTAACTATCAACCTAGTATTTGGAA | Myrcene (Q. ilex) |
| | CCACGATTACATTGAATCACTTCGTATCGAATATGTTGGTGAAACAT | |
| | GTACACGTCAAATTAACGTTTTAAAAGAACAAGTTCGTATGATGTTA | |
| | CACAAAGTTGTTAATCCATTAGAACAATTAGAATTAATTGAAATTTT | |
| | ACAACGTTTAGGTTTAAGTTACCATTTCGAAGAAGAAATAAAACGT | |
| | ATTTTAGATGGTGTTTACAATAACGATCATGGTGGTGATACATGGAA | |
| | AGCAGAAAACCTTTATGCAACAGCTCTTAAATTCCGTCTTTTACGTC | |
| | AGCACGGTTATTCTGTTTCTCAAGAAGTTTTCAACTCTTTTAAAGATG | |
| | AGCGTGGCAGTTTCAAAGCATGTTTATGTGAAGATACTAAAGGTATG | |
| | TTATCACTTTATGAAGCATCTTTCTTTCTTATTGAAGGTGAAAACATT | |
| | TTAGAGGAAGCTAGAGACTTTAGTACAAAACATCTTGAAGAATATG | |
| | TAAAACAAAATAAAGAGAAAAACTTAGCTACTTTAGTTAATCACTC | |
| | ATTAGAATTTCCATTACATTGGCGTATGCCTCGTTTAGAAGCTCGTT | |
| | GGTTCATCAATATCTATCGTCATAATCAAGATGTAAATCCAATCCTT | |
| | TTAGAATTTGCTGAACTTGACTTCAATATTGTACAAGCTGCTCACCA | |
| | AGCAGATTTAAAACAAGTATCAACATGGTGGAAATCAACTGGTTTA | |
| | GTAGAAAATCTTTCATTCGCTCGTGATCGTCCTGTAGAAAACTTCTTT | |
| | TGGACAGTTGGTCTTATTTTCCAACCACAATTCGGTTATTGTCGTAG | |
| | AATGTTTACTAAAGTATTCGCATTAATTACTACAATTGATGACGTAT | |
| | ATGATGTATATGGTACTTTAGATGAATTAGAACTTTTCACAGACGTT | |
| | GTTGAAAGATGGGATATTAATGCAATGGATCAATTACCTGATTATAT | |
| | GAAAATTTGCTTTTTAACATTACACAATAGTGTTAACGAAATGGCAT | |
| | TAGACACTATGAAAGAACAACGTTTTCACATCATTAAATACCTTAAA | |
| | AAAGCATGGGTTGATCTTTGTCGTTATTACTTAGTTGAAGCTAAATG | |
| | GTATAGTAATAAATATAGACCTTCTTTACAAGAATACATTGAAAATG | |
| | CATGGATTTCAATTGGTGCTCCAACTATTTTAGTTCATGCATATTTCT | |
| | TCGTTACAAATCCAATTACAAAAGAAGCATTAGACTGTTTAGAAGA | |
| | ATATCCAAACATTATTCGTTGGAGTAGTATTATTGCACGTTTAGCTG | |
| | ATGATTTAGGTACTTCAACAGACGAATTAAAACGTGGTGACGTACC | |
| | AAAAGCAATTCAATGTTATATGAATGAAACAGGTGCTTCAGAAGAA | |
| | GGTGCTCGTGAGTACATTAAATACTTAATTTCTGCTACTTGGAAAAA | |
| | AATGAACAAAGATAGAGCAGCATCAAGTCCATTTTCACATATCTTCA | |
| | TTGAAATTGCTCTTAATTTAGCACGTATGGCACAATGTTTATATCAA | |
| | CACGGTGACGGCCACGGTTTAGGTAACCGTGAAACAAAAGATCGTA | |
| | TACTTTCATTACTTATTCAACCAATTCCATTAAACAAAGATGGTACC | |
| | GGTGAGAACTTATACTTTCAAGGCTCAGGTGGTGGTGGTTCTGATTA | |
| | CAAAGATGATGATGATAAAGGAACCGGTTAATCTAGACTCGAG | |
| 156 | CATATGGTACCAAGAAGAATTGGAGACTATCACTCAAACTTATGGA | Myrcene (P. abies) |
| | ATGATGACTTCATTCAATCATTAACAACACCATACGGTGCTCCATCA | |
| | TATATTGAACGTGCTGATAGATTAATATCTGAAGTAAAAGAAATGTT | |
| | TAATAGAATGTGTATGGAAGATGGTGAGTTAATGTCTCCATTAAATG | |
| | ATCTTATTCAAAGATTATGGACTGTTGATAGTGTTGAACGTTTAGGT | |
| | ATAGATCGTCACTTCAAAAATGAAATAAAAGCTAGTTTAGATTATGT | |
| | ATACTCATACTGGAACGAAAAAGGTATCGGTTGTGGTCGTCAATCA | |
| | GTAGTTACAGATTTAAACTCTACTGCTCTTGGATTAAGAATTTTACG | |
| | TCAACATGGTTACACAGTTTCAAGTGAAGTTTTAAAAGTTTTTGAAG | |
| | AAGAAAACGGTCAATTTGCTTGTTCACCTTCACAGACTGAGGGCGA | |
| | AATTCGTTCATTCTTAAACTTATATCGTGCTTCATTAATTGCTTTTCC | |
| | TGGTGAAAAAGTAATGGAAGAAGCTCAAATCTTTTCTAGTCGTTACT | |
| | TAAAAGAAGCAGTTCAGAAAATTCCAGTTTCAGGTTTATCTCGTGAA | |
| | ATAGGCGATGTTTTAGAATATGGTTGGCACACAAACTTACCTCGTTG | |
| | GGAAGCTCGTAACTATATGGACGTATTCGGTCAAGACACAAATACA | |
| | TCATTCAACAAAAACAAAATGCAATATATGAATACAGAGAAAATTC | |
| | TTCAATTAGTAAAATTAGAGTTTAATATCTTTCATTCATTACAACAA | |
| | CGTGAATTACAATGTTTATTACGTTGGTGGAAAGAAAGTGGTCTTCC | |
| | ACAATTAACATTTGCACGTCACCGTCACGTTGAATTTTACACTTTAG | |
| | CTTCTTGTATTGCATGTGAACCAAAACACAGTGCATTTCGTTTAGGT | |
| | TTTGCAAAAATGTGTCACTTAGTAACAGTTTTAGATGATGTATATGA | |
| | CACATTTGGCAAAATGGATGAATTAGAACTTTTTACTGCAGCTGTTA | |
| | AACGTTGGGACTTATCAGAAACTGAGCGTTTACCTGAGTATATGAAA | |
| | GGTTTATATGTTGTAGTTTTCGAGACTGTTAATGAATTAGCACAAGA | |
| | AGCAGAGAAAACTCAAGGACGTAATACATTAAATTACGTTCGTAAA | |
| | GCATGGGAAGCATACTTCGATAGTTATATGAAAGAAGCAGAATGGA | |
| | TCTCAACAGGCTATTTACCAACATTCGAAGAGTATTGTGAAAACGGT | |
| | AAAGTATCAAGTGCATATAGAGTTGCTGCACTTCAACCTATTTTAAC | |
| | ATTAGATGTACAACTTCCAGATGACATCTTAAAAGGTATTGATTTTC | |
| | CATCTCGTTTCAATGATTTAGCATCTTCATTTCTTCGTTTACGTGGAG | |
| | ATACTAGATGTTACGAGGCTGATCGTGCTCGTGGTGAAGAAGCAAG | |
| | TTGTATTTCTTGTTACATGAAAGACAATCCAGGTTCAACTGAAGAAG | |
| | ATGCATTAAATCACATTAATGCTATGATAAATGATATTATTCGTGAA | |
| | TTAAACTGGGAATTTCTTAAACCAGACTCAAATATCCCAATGCCAGC | |
| | TCGTAAACATGCTTTCGATATTACAAGAGCTTTACATCACTTATATA | |
| | TTTATCGTGACGGTTTTTCTGTTGCTAACAAAGAGACTAAAAATCTT | |
| | GTTGAGAAAACTTTATTAGAATCAATGTTATTCGGTACCGGTGAGAA | |
| | CCTTTATTTTCAAGGTTCAGGTGGTGGTGGTTCAGATTATAAAGACG | |
| | ATGATGATAAAGGAACCGGTTAATCTAGACTCGAG | |
| 157 | CATATGGTACCAAGAAGATCAGCTAATTATCAACCTAGTCGTTGGGA | Myrcene, |
| | TCATCATCACCTTTTAAGTGTAGAAAACAAATTCGCTAAAGATAAAC | ocimene (A. thalania) |
| | GTGTAAGAGAACGTGACTTACTTAAAGAAAAAGTTCGTAAAATGTT | |
| | AAATGACGAACAGAAAACTTACTTAGATCAATTAGAATTTATTGAC | |
| | GATCTTCAAAAATTAGGTGTTAGTTATCACTTCGAAGCAGAAATAGA | |
| | TAATATACTTACAAGTTCATACAAAAAAGATCGTACAAATATACAA | |
| | GAAAGTGATTTACACGCAACTGCATTAGAGTTTCGTCTTTTTCGTCA | |
| | ACACGGTTTTAACGTTTCAGAAGATGTATTTGATGTATTTATGGAAA | |
| | ATTGTGGTAAATTCGACCGTGATGACATTTATGGTTTAATTTCATTAT | |
| | ATGAAGCTAGTTATCTTTCTACTAAACTTGACAAAAATCTTCAAATC | |
| | TTTATCCGTCCATTTGCTACTCAACAATTACGTGATTTTGTAGATACT | |
| | CACAGTAATGAAGATTTCGGTTCATGTGATATGGTAGAAATAGTTGT | |
| | TCAAGCATTAGACATGCCATACTATTGGCAAATGCGTCGTTTATCTA | |
| | CACGTTGGTATATTGATGTTTATGGTAAAAGACAAAATTACAAAAAC | |
| | TTAGTAGTTGTTGAATTTGCAAAAATTGATTTCAATATTGTTCAAGCT | |
| | ATTCACCAGGAAGAACTTAAAAATGTATCATCTTGGTGGATGGAAA | |
| | CTGGTTTAGGTAAACAACTTTATTTTGCTCGTGATCGTATTGTAGAG | |
| | AACTATTTTTGGACAATTGGTCAAATTCAAGAACCTCAATATGGATA | |
| | TGTTAGACAAACAATGACTAAAATCAATGCTTTATTAACAACAATTG | |
| | ATGATATTTATGATATATACGGTACATTAGAAGAATTACAGTTATTC | |
| | ACAGTTGCATTTGAGAATTGGGACATAAATCGTTTAGACGAATTACC | |
| | AGAATATATGCGTTTATGTTTCTTAGTTATCTATAACGAAGTAAATA | |
| | GTATAGCATGTGAAATTCTTAGAACAAAAAATATTAACGTTATTCCT | |
| | TTCTTAAAAAAATCTTGGACTGATGTAAGTAAAGCATACTTAGTTGA | |
| | AGCTAAATGGTATAAATCAGGCCATAAACCAAATTTAGAAGAGTAT | |
| | ATGCAAAATGCACGTATTTCTATTTCTTCACCAACAATCTTTGTTCAC | |
| | TTTTATTGTGTATTTTCAGACCAATTATCTATTCAAGTTTTAGAAACT | |
| | TTATCACAACACCAACAAAATGTTGTAAGATGTAGTTCTTCTGTTTT | |
| | CCGTTTAGCTAATGACTTAGTAACTTCTCCAGATGAATTAGCTAGAG | |
| | GTGATGTTTGTAAATCAATTCAATGTTATATGTCAGAAACTGGTGCA | |
| | AGTGAAGATAAAGCTAGATCACACGTTCGTCAAATGATTAATGATTT | |
| | ATGGGACGAAATGAATTACGAGAAAATGGCACATTCAAGTAGTATC | |
| | TTACATCATGATTTTATGGAGACAGTAATCAATTTAGCTAGAATGTC | |
| | TCAATGTATGTACCAATATGGTGACGGACACGGTTCTCCAGAAAAA | |
| | GCTAAAATTGTAGATCGTGTAATGAGTTTACTTTTCAACCCTATTCCT | |
| | TTAGATGGTACCGGTGAGAATTTATATTTTCAAGGCTCTGGAGGTGG | |
| | TGGTTCAGATTATAAAGATGATGACGACAAAGGAACCGGTTAATCT | |
| | AGACTCGAG | |
| 158 | CATATGGTACCAAGAAGAAGTGCAAACTATCAACCTTCATTATGGC | Myrcene, |
| | AACATGAATACTTATTATCATTAGGCAACACTTATGTTAAAGAAGAT | ocimene (A. thalania) |
| | AATGTTGAAAGAGTAACTCTTTTAAAACAAGAAGTTTCTAAAATGTT | |
| | AAACGAAACAGAAGGTTTACTTGAACAACTTGAATTAATTGACACTT | |
| | TACAAAGATTAGGTGTTTCTTATCATTTTGAACAGGAGATTAAAAAA | |
| | ACATTAACTAATGTTCATGTTAAAAACGTACGTGCTCATAAAAATCG | |
| | TATTGATCGTAACCGTTGGGGCGATTTATATGCAACTGCATTAGAAT | |
| | TTCGTTTATTACGTCAACATGGTTTTTCTATTGCTCAAGACGTTTTTG | |
| | ATGGTAATATTGGTGTTGACTTAGACGACAAAGACATTAAAGGTATT | |
| | TTAAGTTTATACGAAGCTAGTTACTTATCAACACGTATTGATACAAA | |
| | ACTTAAAGAATCAATCTATTACACAACAAAACGTTTAAGAAAATTC | |
| | GTAGAGGTAAACAAAAACGAAACTAAAAGTTACACTCTTCGTCGTA | |
| | TGGTTATTCACGCACTTGAGATGCCTTATCACCGTCGTGTTGGTCGTC | |
| | TTGAAGCTCGTTGGTATATCGAGGTATATGGAGAAAGACACGACAT | |
| | GAATCCTATTTTATTAGAATTAGCTAAATTAGATTTTAACTTTGTTCA | |
| | GGCTATCCACCAAGACGAATTAAAATCATTATCTAGTTGGTGGTCTA | |
| | AAACAGGATTAACAAAACATTTAGACTTTGTTCGTGATCGTATTACA | |
| | GAGGGTTACTTCAGTAGTGTAGGTGTTATGTATGAACCAGAATTTGC | |
| | ATATCATCGTCAAATGCTTACAAAAGTATTTATGCTTATTACAACTA | |
| | TTGATGACATCTATGACATTTACGGTACACTTGAAGAATTACAATTA | |
| | TTCACAACTATCGTTGAAAAATGGGATGTTAATCGTTTAGAAGAACT | |
| | TCCTAACTATATGAAATTATGCTTCTTATGTTTAGTTAACGAAATAA | |
| | ATCAAATTGGATATTTTGTATTAAGAGATAAAGGTTTTAATGTAATT | |
| | CCTTATCTTAAAGAGTCTTGGGCTGACATGTGTACTACATTTCTTAA | |
| | AGAAGCTAAATGGTACAAATCAGGTTATAAACCAAATTTTGAAGAG | |
| | TATATGCAAAATGGCTGGATTTCATCATCAGTTCCAACTATTCTTTTA | |
| | CACTTATTTTGTTTATTAAGTGACCAAACTTTAGACATTCTTGGTTCT | |
| | TATAATCACAGTGTTGTTCGTAGTTCAGCAACAATTTTACGTCTTGC | |
| | AAATGATTTAGCTACTTCTTCAGAAGAATTAGCAAGAGGAGATACA | |
| | ATGAAATCAGTTCAATGTCACATGCATGAAACTGGTGCTTCAGAAGC | |
| | TGAATCAAGAGCTTACATTCAAGGTATTATTGGCGTAGCTTGGGATG | |
| | ACCTTAATATGGAGAAAAAATCATGTCGTTTACACCAGGGATTCTTA | |
| | GAAGCAGCAGCAAATTTAGGACGTGTAGCACAATGCGTATATCAAT | |
| | ATGGAGACGGTCACGGTTGTCCAGATAAAGCAAAAACAGTAAATCA | |
| | TGTTCGTAGTTTATTAGTTCACCCATTACCATTAAACGGTACCGGTG | |
| | AAAACCTTTATTTTCAAGGTAGTGGTGGAGGTGGTTCTGATTATAAA | |
| | GACGACGATGACAAAGGAACCGGTTAATCTAGACTCGAG | |
| 159 | CATATGGTACCAGCTTCTCCACCTGCTCATCGTTCATCTAAAGCAGC | Sesquiterpene |
| | AGACGAAGAGTTACCAAAAGCATCTTCTACATTCCATCCATCTCTTT | (Z. mays; |
| | GGGGTTCATTTTTCTTAACATATCAGCCACCTACAGCTCCACAACGT | B73) |
| | GCAAATATGAAAGAACGTGCTGAAGTTCTTCGTGAACGTGTTCGTAA | |
| | AGTATTAAAAGGTTCAACAACAGATCAATTACCTGAAACAGTTAAC | |
| | TTAATTCTTACATTACAAAGACTTGGTTTAGGTTATTACTATGAAAA | |
| | TGAAATTGACAAATTACTTCATCAAATTTACTCTAATTCAGATTATA | |
| | ACGTAAAAGACTTAAACTTAGTTTCTCAACGTTTTTACTTACTTCGTA | |
| | AAAACGGTTATGACGTACCTTCTGATGTTTTCTTATCTTTTAAAACTG | |
| | AAGAAGGTGGTTTCGCTTGTGCTGCAGCTGACACACGTTCACTTTTA | |
| | AGTTTATACAATGCTGCTTACCTTCGTAAACATGGTGAAGAAGTATT | |
| | AGATGAAGCAATTTCATCAACACGTTTAAGATTACAAGACTTATTAG | |
| | GTCGTTTATTACCTGAATCACCATTCGCTAAAGAAGTATCAAGTTCA | |
| | CTTCGTACACCTTTATTCCGTCGTGTAGGTATTTTAGAAGCTCGTAAC | |
| | TATATTCCAATCTATGAAACTGAAGCTACAAGAAATGAAGCTGTATT | |
| | AGAGCTTGCTAAACTTAACTTCAATTTACAACAGCTTGATTTCTGTG | |
| | AAGAATTAAAACATTGTAGTGCATGGTGGAATGAGATGATTGCTAA | |
| | AAGTAAATTAACTTTTGTACGTGACCGTATAGTTGAAGAATACTTTT | |
| | GGATGAATGGTGCATGTTATGATCCACCATATTCATTAAGTCGTATT | |
| | ATTCTTACAAAAATCACTGGTTTAATTACTATTATTGATGATATGTTC | |
| | GATACTCATGGTACAACAGAGGATTGCATGAAATTCGCAGAAGCAT | |
| | TTGGTCGTTGGGATGAATCAGCAATTCATCTTCTTCCAGAATACATG | |
| | AAAGATTTTTACATTTTAATGTTAGAAACTTTCCAGTCATTTGAAGA | |
| | TGCACTTGGTCCAGAAAAATCATACCGTGTATTATACTTAAAACAAG | |
| | CAATGGAACGTTTAGTAGAGTTATATTCTAAAGAAATCAAATGGCGT | |
| | GATGACGATTATGTTCCAACAATGTCAGAACATTTACAAGTTAGTGC | |
| | TGAAACAATTGCTACAATTGCTTTAACTTGCTCTGCTTATGCTGGTAT | |
| | GGGTGATATGTCTATTCGTAAAGAAACATTTGAATGGGCATTATCTT | |
| | TCCCTCAATTCATTAGAACTTTTGGTTCATTTGTACGTTTATCAAATG | |
| | ATGTTGTATCAACAAAACGTGAACAAACTAAAGATCATTCACCTTCA | |
| | ACAGTTCACTGTTATATGAAAGAACACGGTACAACTATGGACGATG | |
| | CTTGTGAAAAAATCAAAGAATTAATTGAGGACTCATGGAAAGACAT | |
| | GTTAGAACAATCTTTAGCTCTTAAAGGCTTACCTAAAGTAGTACCTC | |
| | AATTAGTTTTTGATTTCTCTCGTACTACAGATAACATGTATCGTGACC | |
| | GTGATGCTTTAACATCATCAGAAGCATTAAAAGAAATGATACAGTT | |
| | ATTATTCGTAGAACCTATACCTGAAGGTACCGGTGAGAATCTTTATT | |
| | TTCAAGGATCAGGTGGTGGAGGCTCAGATTACAAAGATGACGACGA | |
| | TAAAGGAACCGGTTAATCTAGACTCGAG | |
| 160 | CATATGGTACCAGAGGCTTTAGGAAATTTTGATTATGAGAGTTATAC | Sesquiterpene |
| | TAATTTTACAAAATTACCATCATCACAATGGGGTGATCAATTCCTTA | (A. thalania) |
| | AATTTTCTATAGCAGATTCTGACTTCGATGTATTAGAAAGAGAAATA | |
| | GAAGTATTAAAACCAAAAGTAAGAGAGAACATTTTTGTTTCATCAA | |
| | GTACTGATAAAGATGCAATGAAAAAAACAATTTTAAGTATTCATTTC | |
| | TTAGATAGTTTAGGTTTATCTTATCACTTCGAAAAAGAAATAGAGGA | |
| | GAGTTTAAAACATGCTTTCGAGAAAATTGAAGACCTTATTGCTGATG | |
| | AAAATAAACTTCATACAATAAGTACAATTTTCCGTGTATTCCGTACA | |
| | TACGGCTATTATATGTCTTCTGATGTATTCAAAATTTTCAAAGGAGA | |
| | CGATGGTAAATTCAAAGAAAGTTTAATTGAAGACGTTAAAGGTATG | |
| | CTTTCTTTTTATGAAGCTGTTCATTTTGGAACAACTACTGATCACATT | |
| | TTAGACGAAGCTCTTAGTTTTACATTAAACCACTTAGAGTCACTTGC | |
| | AACAGGCCGTCGTGCATCACCACCACATATTAGTAAATTAATCCAAA | |
| | ATGCTTTACATATTCCTCAACATCGTAACATCCAGGCATTAGTAGCT | |
| | CGTGAATACATTAGTTTTTACGAACACGAAGAAGATCACGATGAAA | |
| | CATTATTAAAATTAGCTAAATTAAACTTTAAATTCTTACAACTTCACT | |
| | ATTTTCAAGAATTAAAAACAATTACAATGTGGTGGACTAAATTAGAT | |
| | CATACATCTAATTTACCACCAAATTTTCGTGAACGTACAGTTGAAAC | |
| | ATGGTTTGCAGCTTTAATGATGTATTTCGAACCACAATTTAGTTTAG | |
| | GTCGTATTATGAGTGCAAAATTATATTTAGTAATTACTTTCTTAGATG | |
| | ACGCATGTGATACATACGGATCAATATCTGAAGTAGAGTCATTAGCT | |
| | GATTGTTTAGAACGTTGGGACCCAGATTATATGGAAAATTTACAAGG | |
| | TCACATGAAAACAGCATTCAAATTCGTTATGTATTTATTCAAAGAAT | |
| | ACGAAGAAATTTTACGTTCACAAGGCCGTTCATTCGTATTAGAGAAA | |
| | ATGATTGAGGAGTTTAAAATTATCGCACGTAAAAACTTAGAACTTGT | |
| | AAAATGGGCTCGTGGTGGTCACGTTCCTTCTTTTGACGAATATATAG | |
| | AGAGTGGTGGTGCTGAGATTGGTACTTATGCTACAATCGCTTGTTCA | |
| | ATTATGGGTCTTGGTGAAATTGGTAAAAAAGAAGCATTTGAGTGGTT | |
| | AATCTCTCGTCCTAAACTTGTTCGTATTTTAGGTGCTAAAACACGTTT | |
| | AATGGATGATATCGCAGACTTTGAAGAAGACATGGAAAAAGGCTAT | |
| | ACAGCTAATGCACTTAACTATTATATGAATGAACACGGAGTAACTA | |
| | AAGAAGAAGCTAGTCGTGAACTTGAGAAAATGAATGGTGATATGAA | |
| | CAAAATTGTAAACGAAGAATGTCTTAAAATTACAACTATGCCACGTC | |
| | GTATCTTAATGCAAAGTGTTAACTACGCTCGTAGTTTAGATGTATTA | |
| | TACACAGCTGATGATGTATATAACCACCGTGAAGGCAAACTTAAAG | |
| | AATATATGAGATTACTTTTAGTAGATCCAATTTTACTTGGTACCGGT | |
| | GAAAATCTTTATTTTCAAGGTTCAGGTGGTGGTGGTTCTGATTATAA | |
| | AGATGATGACGATAAAGGAACCGGTTAATCTAGACTCGAG | |
| 161 | CATATGGTACCAGAGAGTCAAACAACATTCAAATACGAATCATTAG | Sesquiterpene |
| | CATTTACAAAACTTAGTCACTGTCAATGGACAGACTATTTTCTTAGT | (A. thalania) |
| | GTTCCAATTGATGAAAGTGAATTAGATGTTATTACTCGTGAAATTGA | |
| | TATTCTTAAACCAGAAGTTATGGAGTTATTAAGTAGTCAAGGAGATG | |
| | ATGAAACAAGTAAAAGAAAAGTTCTTCTTATTCAGTTATTACTTTCT | |
| | TTAGGTTTAGCATTCCACTTTGAAAATGAGATTAAAAACATACTTGA | |
| | ACACGCATTTCGTAAAATAGATGATATAACTGGTGACGAAAAAGAC | |
| | TTATCAACAATTAGTATTATGTTCCGTGTTTTCCGTACTTATGGACAC | |
| | AATCTTCCAAGTAGTGTTTTTAAACGTTTCACAGGTGATGATGGTAA | |
| | ATTTCAGCAAAGTTTAACAGAAGACGCAAAAGGTATTTTAAGTTTAT | |
| | ATGAAGCTGCACATTTAGGTACTACTACAGATTACATTTTAGATGAA | |
| | GCTCTTAAATTCACATCTAGTCACTTAAAAAGTTTACTTGCTGGTGG | |
| | TACATGTCGTCCTCACATCTTACGTTTAATCCGTAATACATTATACTT | |
| | ACCACAACGTTGGAACATGGAAGCTGTTATCGCTCGTGAATACATAT | |
| | CATTTTACGAGCAGGAAGAAGATCACGATAAAATGCTTTTACGTCTT | |
| | GCAAAACTTAACTTTAAACTTCTTCAATTACACTACATTAAAGAGCT | |
| | TAAAAGTTTCATTAAATGGTGGATGGAACTTGGTTTAACTTCTAAAT | |
| | GGCCTTCTCAATTTCGTGAACGTATTGTTGAAGCATGGTTAGCTGGA | |
| | TTAATGATGTATTTTGAACCACAGTTCTCAGGTGGTCGTGTTATTGCT | |
| | GCAAAATTCAACTATTTACTTACAATATTAGACGACGCATGTGACCA | |
| | CTATTTTTCTATTCACGAATTAACACGTTTAGTTGCATGTGTAGAACG | |
| | TTGGTCACCAGATGGTATTGACACATTAGAAGATATTTCACGTTCTG | |
| | TATTCAAATTAATGTTAGATGTTTTCGACGATATTGGTAAAGGTGTA | |
| | CGTTCAGAAGGTTCTAGTTACCACTTAAAAGAAATGTTAGAGGAATT | |
| | AAACACTTTAGTTCGTGCTAATTTAGATTTAGTTAAATGGGCTCGTG | |
| | GAATACAAACAGCTGGTAAAGAGGCTTATGAATGGGTTCGTTCACG | |
| | TCCACGTTTAATCAAATCTTTAGCAGCTAAAGGTAGACTTATGGATG | |
| | ATATTACAGACTTTGACTCAGATATGAGTAATGGATTCGCAGCTAAT | |
| | GCTATTAACTACTATATGAAACAATTTGTTGTTACAAAAGAAGAAGC | |
| | TATTCTTGAATGTCAACGTATGATTGTAGACATTAACAAAACTATTA | |
| | ATGAAGAGTTATTAAAAACTACTTCAGTTCCAGGTCGTGTATTAAAA | |
| | CAAGCTCTTAACTTTGGCCGTTTATTAGAATTATTATATACAAAATCT | |
| | GACGATATTTACAATTGTTCTGAAGGCAAACTTAAAGAATACATTGT | |
| | AACTCTTTTAATTGATCCTATAAGACTTGGTACCGGTGAAAACTTAT | |
| | ACTTTCAAGGTTCAGGCGGTGGTGGTAGTGATTACAAAGATGATGAT | |
| | GACAAAGGAACCGGTTAATCTAGACTCGAG | |
| 162 | CATATGGTACCAGAGAGTCAAACAAAATTCGACTACGAATCATTAG | Sesquiterpene |
| | CTTTTACAAAATTATCACATTCACAATGGACTGATTACTTTTTATCAG | (A. thalania) |
| | TACCTATAGACGACTCTGAACTTGACGCAATTACTCGTGAAATCGAC | |
| | ATTATCAAACCTGAAGTTCGTAAATTACTTTCAAGTAAAGGTGATGA | |
| | TGAAACTTCTAAACGTAAAGTATTACTTATCCAAAGTTTATTATCAT | |
| | TAGGTTTAGCATTTCATTTTGAAAACGAAATTAAAGATATTTTAGAA | |
| | GATGCATTTAGACGTATTGATGACATTACAGGTGATGAAAACGACTT | |
| | AAGTACTATTAGTATTATGTTCCGTGTATTCCGTACATACGGTCACA | |
| | ATTTACCAAGTAGTGTTTTTAAACGTTTCACTGGTGATGACGGTAAA | |
| | TTTGAACGTTCTTTAACTGAAGATGCTAAAGGAATTTTATCATTATA | |
| | TGAAGCTGCACATTTAGGAACAACTACTGATTATATTCTTGATGAAG | |
| | CATTAGAATTTACTTCATCACACTTAAAATCTTTACTTGTTGGTGGTA | |
| | TGTGTCGTCCACATATTTTACGTCTTATTAGAAATACTTTATATCTTC | |
| | CACAACGTTGGAATATGGAAGCAGTAATTGCAAGAGAATACATTAG | |
| | TTTTTATGAACAAGAAGAAGATCACGATAAAATGTTACTTCGTTTAG | |
| | CTAAATTAAATTTCAAATTACTTCAATTACACTACATTAAAGAGTTA | |
| | AAAACATTCATTAAATGGTGGATGGAATTAGGACTTACATCAAAAT | |
| | GGCCTTCTCAATTTCGTGAACGTATTGTTGAAGCATGGTTAGCTGGT | |
| | CTTATGATGTATTTTGAACCACAGTTTTCTGGAGGTCGTGTAATAGC | |
| | TGCTAAATTCAATTACTTATTAACAATTTTAGATGATGCATGTGATC | |
| | ACTATTTCTCAATTCCAGAATTAACTCGTTTAGTTGATTGCGTAGAA | |
| | AGATGGAATCATGATGGTATACATACTTTAGAAGACATCTCACGTAT | |
| | CATCTTTAAACTTGCATTAGATGTATTTGATGATATTGGTCGTGGTGT | |
| | TCGTTCTAAAGGTTGTTCTTATTACTTAAAAGAAATGTTAGAAGAGT | |
| | TAAAAATCTTAGTTCGTGCAAACTTAGATTTAGTTAAATGGGCTCGT | |
| | GGTAATCAATTACCTAGTTTTGAAGAACACGTTGAGGTAGGTGGTAT | |
| | TGCTCTTACAACATACGCAACTTTAATGTACTCTTTTGTTGGCATGGG | |
| | TGAAGCAGTAGGTAAAGAAGCATACGAATGGGTACGTTCTCGTCCA | |
| | CGTTTAATCAAAAGTTTAGCAGCAAAAGGTCGTCTTATGGACGATAT | |
| | TACTGATTTCGAAGTAAAAATTATCAACTTATTTTTCGACCTTCTTTT | |
| | ATTTGTATTCGGTACCGGTGAAAACTTATATTTCCAGGGTAGTGGTG | |
| | GAGGAGGTTCAGACTACAAAGATGACGATGACAAAGGAACCGGTTA | |
| | ATCTAGACTCGAG | |
| 163 | CATATGGTACCAGCAGCTTTCACAGCAAATGCAGTTGACATGCGTCC | Curcumene |
| | ACCAGTTATTACAATTCACCCACGTTCAAAAGATATTTTCTCTCAATT | (P. cablin) |
| | TTCTTTAGATGATAAATTACAAAAACAATACGCTCAAGGAATCGAA | |
| | GCTCTTAAAGAAGAAGCTCGTTCTATGCTTATGGCTGCAAAATCTGC | |
| | TAAAGTAATGATCTTAATTGATACACTTGAACGTTTAGGATTAGGTT | |
| | ATCACTTTGAAAAAGAAATTGAAGAGAAATTAGAAGCTATTTACAA | |
| | AAAAGAGGATGGTGACGATTATGATCTTTTTACAACTGCTTTAAGAT | |
| | TCCGTTTACTTAGACAACACCAACGTCGTGTACCATGTTCTGTTTTTG | |
| | ACAAATTTATGAATAAAGAGGGTAAATTCGAAGAAGAACCATTAAT | |
| | TTCAGATGTTGAAGGTCTTCTTTCATTATATGACGCTGCTTATTTACA | |
| | GATTCACGGTGAACACATTTTACAAGAGGCTTTAATTTTCACTACAC | |
| | ATCATTTAACTCGTATTGAACCACAATTAGATGATCACTCTCCTTTA | |
| | AAATTAAAATTAAACCGTGCTTTAGAATTTCCTTTTTACAGAGAAAT | |
| | CCCTATAATCTATGCACATTTTTACATTTCAGTATATGAACGTGACG | |
| | ATTCTCGTGATGAAGTATTATTAAAAATGGCTAAATTATCTTATAAT | |
| | TTCTTACAAAACTTATACAAAAAAGAATTAAGTCAACTTTCTCGTTG | |
| | GTGGAACAAATTAGAACTTATTCCTAATTTACCTTATATTCGTGATTC | |
| | TGTAGCTGGAGCTTATTTATGGGCTGTTGCTTTATATTTCGAACCTCA | |
| | ATATTCAGACGTTCGTATGGCAATTGCTAAACTTATCCAAATTGCAG | |
| | CAGCTGTAGATGATACTTACGATAATTATGCTACTATACGTGAAGCT | |
| | CAATTATTAACAGAAGCATTAGAACGTTTAAATGTACACGAAATTG | |
| | ACACATTACCAGATTATATGAAAATTGTTTATCGTTTTGTAATGTCAT | |
| | GGAGTGAAGATTTCGAACGTGATGCTACAATTAAAGAACAGATGTT | |
| | AGCTACACCTTATTTCAAAGCTGAAATGAAAAAACTTGGTCGTGCTT | |
| | ATAATCAAGAACTTAAATGGGTTATGGAACGTCAATTACCTAGTTTC | |
| | GAAGAATACATGAAAAACTCTGAAATCACTTCTGGTGTTTACATTAT | |
| | GTTTACTGTAATTAGTCCTTACTTAAATAGTGCAACACAAAAAAACA | |
| | TTGACTGGTTATTATCACAACCTCGTTTAGCATCTTCAACTGCAATTG | |
| | TTATGCGTTGTTGTAATGATTTAGGCTCTAATCAACGTGAATCTAAA | |
| | GGAGGAGAAGTTATGACATCTTTAGATTGCTATATGAAACAACACG | |
| | GTGCTAGTAAACAAGAAACAATTTCTAAATTCAAACTTATTATCGAA | |
| | GATGAATGGAAAAACTTAAATGAAGAATGGGCTGCAACAACATGTC | |
| | TTCCAAAAGTTATGGTAGAAATTTTTCGTAACTATGCACGTATTGCA | |
| | GGCTTTTGCTACAAAAATAACGGTGATGCTTATACATCTCCAAAAAT | |
| | TGTACAACAATGTTTTGACGCTTTATTTGTAAATCCATTAAGAATTG | |
| | GTACCGGTGAGAATTTATACTTTCAAGGCTCAGGTGGAGGTGGTAGT | |
| | GATTATAAAGATGATGATGATAAAGGAACCGGTTAATCTAGACTCG | |
| | AG | |
| 164 | CATATGGTACCAGAATTTAGAGTTCATTTACAGGCTGATAATGAACA | Farnesene |
| | GAAAATATTCCAGAACCAAATGAAACCTGAACCTGAAGCATCATAT | (M. domestica) |
| | CTTATTAATCAACGTAGATCAGCTAATTACAAACCTAATATTTGGAA | |
| | AAATGACTTTTTAGATCAAAGTTTAATTAGTAAATACGACGGTGATG | |
| | AATATCGTAAATTAAGTGAGAAATTAATCGAGGAAGTAAAAATTTA | |
| | TATATCTGCTGAGACAATGGACTTAGTAGCTAAATTAGAACTTATTG | |
| | ATTCTGTTCGTAAATTAGGTTTAGCTAATCTTTTTGAAAAAGAAATT | |
| | AAAGAAGCATTAGATTCTATCGCAGCTATTGAGTCAGATAATTTAGG | |
| | TACTCGTGATGACTTATATGGTACTGCTTTACACTTTAAAATTTTACG | |
| | TCAACATGGTTATAAAGTTTCTCAAGATATTTTTGGTCGTTTCATGGA | |
| | TGAAAAAGGTACATTAGAAAATCATCACTTCGCTCACTTAAAAGGT | |
| | ATGTTAGAATTATTTGAAGCATCTAATTTAGGTTTTGAAGGTGAAGA | |
| | TATTTTAGATGAAGCAAAAGCATCACTTACATTAGCTCTTCGTGATA | |
| | GTGGTCATATTTGTTATCCAGATTCTAACTTAAGTCGTGATGTAGTA | |
| | CACTCATTAGAATTACCTAGTCACCGTCGTGTTCAATGGTTTGATGTT | |
| | AAATGGCAAATTAATGCTTATGAAAAAGATATTTGTAGAGTTAATGC | |
| | AACTCTTTTAGAATTAGCAAAATTAAATTTTAACGTAGTACAAGCAC | |
| | AACTTCAAAAAAACTTACGTGAAGCATCTCGTTGGTGGGCTAACTTA | |
| | GGTTTCGCTGATAACTTAAAATTCGCTCGTGATCGTTTAGTTGAATG | |
| | TTTTTCTTGCGCAGTAGGCGTAGCATTTGAACCTGAACACTCTTCTTT | |
| | TCGTATCTGTTTAACAAAAGTTATTAATTTAGTTTTAATAATTGATGA | |
| | CGTATACGACATATATGGAAGTGAAGAAGAATTAAAACACTTTACA | |
| | AATGCTGTTGATCGTTGGGATTCTCGTGAAACAGAACAATTACCAGA | |
| | ATGTATGAAAATGTGCTTTCAAGTTTTATACAATACTACATGTGAAA | |
| | TTGCTCGTGAAATTGAAGAAGAAAATGGATGGAATCAAGTTTTACCT | |
| | CAATTAACTAAAGTATGGGCTGATTTTTGTAAAGCATTATTAGTAGA | |
| | AGCTGAATGGTACAATAAAAGTCACATCCCAACTTTAGAAGAATAT | |
| | CTTCGTAATGGCTGTATTTCATCAAGTGTTTCTGTATTATTAGTACAT | |
| | TCTTTCTTTAGTATTACACATGAAGGTACAAAAGAAATGGCAGATTT | |
| | CTTACACAAAAACGAAGACTTATTATACAACATCTCATTAATTGTAC | |
| | GTTTAAACAACGACTTAGGTACAAGTGCAGCTGAACAAGAACGTGG | |
| | TGATTCACCATCATCTATTGTATGTTACATGCGTGAAGTTAATGCTA | |
| | GTGAAGAAACAGCTCGTAAAAATATAAAAGGAATGATCGACAATGC | |
| | TTGGAAAAAAGTTAATGGTAAATGTTTTACAACTAATCAAGTTCCTT | |
| | TTCTTTCTTCTTTTATGAATAACGCTACTAATATGGCTCGTGTAGCTC | |
| | ATTCATTATATAAAGACGGAGACGGTTTTGGCGATCAGGAAAAAGG | |
| | TCCACGTACTCACATCTTATCTTTATTATTCCAACCATTAGTTAACGG | |
| | TACCGGTGAAAACTTATACTTTCAAGGTTCTGGTGGTGGTGGTTCTG | |
| | ACTACAAAGATGACGATGACAAAGGAACCGGTTAATCTAGACTCGAG | |
| 165 | CATATGGTACCAAGTAGTAATGTATCAGCTATTCCTAATTCTTTTGA | Farnesene |
| | ATTAATTCGTCGTTCAGCTCAATTTCAGGCTTCTGTATGGGGTGATTA | (C. sativus) |
| | CTTTTTATCTTATCACTCTTTACCACCTGAGAAAGGTAATAAAGTAA | |
| | TGGAAAAACAAACTGAAGAACTTAAAGAGGAAATCAAAATGGAATT | |
| | AGTTTCTACTACTAAAGATGAACCAGAGAAATTACGTTTAATTGACC | |
| | TTATTCAACGTTTAGGTGTATGTTATCACTTTGAAAATGAAATTAAC | |
| | AACATTTTACAACAATTACACCACATTACTATTACTTCTGAGAAAAA | |
| | CGGTGACGATAATCCTTATAACATGACTTTATGTTTCCGTTTATTACG | |
| | TCAACAAGGTTACAATGTATCTAGTGAACCTTTTGATCGTTTTCGTG | |
| | GCAAATGGGAATCTTCTTATGATAACAATGTAGAAGAACTTTTATCA | |
| | TTATATGAAGCATCTCAATTAAGAATGCAAGGTGAAGAAGCATTAG | |
| | ATGAAGCATTCTGTTTTGCAACTGCACAATTAGAAGCTATTGTTCAA | |
| | GATCCTACTACAGATCCAATGGTTGCAGCAGAAATCAGACAAGCAT | |
| | TAAAATGGCCAATGTACAAAAACTTACCTCGTTTAAAAGCTCGTCAT | |
| | CATATTGGTTTATATTCTGAGAAACCATGGCGTAATGAGTCATTACT | |
| | TAATTTCGCAAAAATGGACTTCAATAAACTTCAAAATTTACATCAAA | |
| | CTGAAATTGCATATATTTCTAAATGGTGGGACGATTACGGCTTTGCA | |
| | GAAAAACTTTCTTTCGCACGTAATCGTATTGTTGAAGGCTATTTCTTC | |
| | GCATTAGGTATCTTTTTCGAACCTCAACTTTTAACAGCACGTCTTATA | |
| | ATGACAAAAGTAATCGCTATTGGTTCTATGTTAGATGACATTTATGA | |
| | TGTTTATGGTACTTTTGAAGAGTTAAAACTTTTAACATTAGCTTTAGA | |
| | ACGTTGGGATAAATCAGAAACAAAACAATTACCTAATTACATGAAA | |
| | ATGTACTACGAAGCATTATTAGATGTTTTTGAAGAAATTGAGCAAGA | |
| | AATGTCACAAAAAGAAACTGAAACAACACCATACTGTATTCATCAC | |
| | ATGAAAGAAGCTACTAAAGAACTTGGACGTGTATTTTTAGTTGAAGC | |
| | AACTTGGTGTAAAGAAGGTTATACTCCTAAAGTAGAGGAATACTTA | |
| | GACATTGCTTTAATTTCTTTTGGTCATAAATTACTTATGGTAACTGCT | |
| | TTATTAGGTATGGGTTCTCACATGGCTACACAACAAATTGTACAATG | |
| | GATTACATCTATGCCAAATATCTTAAAAGCATCTGCAGTAATATGTC | |
| | GTTTAATGAATGACATTGTATCTCATAAATTTGAACAAGAACGTGGT | |
| | CATGTTGCTTCTGCTATCGAATGCTACATGGAACAAAACCACCTTAG | |
| | TGAATATGAAGCATTAATTGCTCTTCGTAAACAAATTGATGATTTAT | |
| | GGAAAGACATGGTAGAAAATTACTGTGCAGTAATCACAGAAGACGA | |
| | AGTACCTCGTGGTGTTTTAATGCGTGTTTTAAATCTTACACGTTTATT | |
| | CAATGTTATTTACAAAGACGGTGATGGATACACACAAAGTCATGGT | |
| | AGTACAAAAGCTCACATTAAAAGTCTTTTAGTTGATAGTGTACCTCT | |
| | TGGTACCGGTGAAAATCTTTACTTTCAAGGTTCAGGTGGAGGTGGTT | |
| | CTGATTATAAAGATGATGATGACAAAGGAACCGGTTAATCTAGACT | |
| | CGAG | |
| 166 | CATATGGTACCAAAAGACATGAGTATTCCATTATTAGCAGCTGTATC | Farnesene |
| | TTCTAGTACAGAAGAAACAGTACGTCCTATCGCAGATTTTCATCCAA | (C. junos) |
| | CACTTTGGGGTAATCATTTTCTTAAATCTGCTGCTGACGTAGAAACT | |
| | ATTGATGCAGCAACACAAGAGCAACACGCTGCATTAAAACAAGAAG | |
| | TACGTCGTATGATTACTACAACAGCAAATAAACTTGCACAAAAACTT | |
| | CACATGATTGATGCTGTACAACGTTTAGGTGTTGCTTATCATTTTGA | |
| | AAAAGAAATTGAAGACGAATTAGGTAAAGTAAGTCACGATTTAGAT | |
| | TCAGATGATTTATACGTTGTATCTTTACGTTTTCGTTTATTCCGTCAA | |
| | CAAGGTGTAAAAATTAGTTGCGATGTTTTCGACAAATTCAAAGATGA | |
| | CGAAGGAAAATTCAAAGAGTCTCTTATTAACGATATTAGAGGAATG | |
| | TTATCATTATACGAAGCAGCTTACTTAGCTATTAGAGGTGAAGATAT | |
| | TTTAGACGAAGCAATTGTTTTCACAACTACTCACTTAAAAAGTGTTA | |
| | TCTCTATTAGTGATCATTCACATGCTAATAGTAATTTAGCTGAACAA | |
| | ATACGTCATAGTTTACAAATTCCACTTCGTAAAGCTGCTGCAAGATT | |
| | AGAAGCACGTTATTTCTTAGATATTTACTCTCGTGATGATTTACATG | |
| | ATGAAACATTACTTAAATTCGCTAAACTTGACTTTAACATTCTTCAA | |
| | GCTGCACACCAAAAAGAAGCTAGTATTATGACTCGTTGGTGGAACG | |
| | ATTTAGGTTTTCCTAAAAAAGTTCCTTATGCTCGTGACCGTATTATAG | |
| | AAACTTATATTTGGATGTTATTAGGAGTTTCATACGAACCTAATTTA | |
| | GCATTTGGAAGAATTTTTGCAAGTAAAGTAGTATGTATGATTACAAC | |
| | AATTGATGATACATTTGATGCTTATGGTACATTTGAAGAGTTAACAT | |
| | TATTCACTGAAGCTGTTACACGTTGGGATATTGGTTTAATTGACACA | |
| | TTACCTGAATATATGAAATTCATTGTAAAAGCTCTTTTAGACATTTA | |
| | CCGTGAAGCTGAAGAAGAATTAGCTAAAGAAGGTAGATCATACGGT | |
| | ATTCCATACGCTAAACAAATGATGCAAGAGTTAATCATTTTATACTT | |
| | TACTGAGGCTAAATGGTTATACAAAGGTTACGTTCCTACATTTGACG | |
| | AATACAAAAGTGTAGCTTTACGTTCTATTGGTCTTAGAACATTAGCA | |
| | GTAGCTTCATTTGTAGATTTAGGTGACTTTATTGCTACAAAAGACAA | |
| | TTTTGAATGTATTCTTAAAAATGCAAAAAGTTTAAAAGCTACTGAAA | |
| | CAATTGGCCGTTTAATGGATGATATAGCTGGTTACAAATTTGAACAG | |
| | AAACGTGGTCATAACCCATCTGCTGTTGAGTGTTACAAAAATCAACA | |
| | CGGAGTATCAGAAGAAGAAGCAGTTAAAGAGCTTTTATTAGAAGTT | |
| | GCAAACAGTTGGAAAGATATTAACGAGGAACTTTTAAATCCAACTA | |
| | CAGTTCCATTACCTATGTTACAGCGTTTATTATATTTTGCTCGTTCAG | |
| | GTCACTTCATCTATGATGATGGACATGATCGTTATACACATTCTTTA | |
| | ATGATGAAAAGACAAGTTGCACTTTTATTAACTGAACCTTTAGCTAT | |
| | TGGTACCGGTGAAAACTTATACTTTCAAGGTTCAGGTGGTGGTGGAT | |
| | CTGATTATAAAGATGATGATGACAAAGGAACCGGTTAATCTAGACT | |
| | CGAG | |
| 167 | CATATGGTACCAGATTTAGCTGTTGAGATTGCAATGGACTTAGCTGT | Farnesene |
| | TGATGACGTTGAGCGTCGTGTAGGTGACTATCATAGTAACCTTTGGG | (P. abies) |
| | ATGATGATTTTATTCAGAGTTTATCAACACCATACGGCGCATCATCA | |
| | TATCGTGAACGTGCTGAAAGATTAGTAGGAGAAGTTAAAGAAATGT | |
| | TTACTTCTATTTCTATCGAAGATGGTGAACTTACATCTGATTTATTAC | |
| | AACGTTTATGGATGGTAGATAATGTAGAGCGTTTAGGCATTTCACGT | |
| | CATTTCGAGAACGAAATAAAAGCAGCTATTGATTATGTTTATTCATA | |
| | TTGGAGTGACAAAGGTATTGTACGTGGTCGTGATTCAGCTGTTCCTG | |
| | ACTTAAATAGTATTGCTTTAGGTTTTCGTACATTACGTTTACACGGTT | |
| | ACACAGTTAGTAGTGATGTATTTAAAGTTTTCCAAGATCGTAAAGGT | |
| | GAATTTGCTTGCAGTGCAATTCCAACTGAAGGAGATATTAAAGGAG | |
| | TTTTAAACTTACTTCGTGCAAGTTATATTGCATTCCCTGGTGAAAAA | |
| | GTAATGGAAAAAGCTCAAACTTTTGCAGCAACATACCTTAAAGAAG | |
| | CATTACAGAAAATTCAAGTAAGTAGTTTAAGTCGTGAAATCGAATAT | |
| | GTTCTTGAATACGGTTGGTTAACTAACTTTCCTCGTTTAGAAGCACG | |
| | TAACTATATTGACGTATTCGGTGAAGAAATTTGTCCATACTTCAAAA | |
| | AACCATGTATTATGGTTGACAAACTTTTAGAATTAGCAAAATTAGAA | |
| | TTTAACTTATTTCACAGTCTTCAACAAACAGAGTTAAAACATGTTAG | |
| | TCGTTGGTGGAAAGATAGTGGTTTCTCTCAATTAACATTTACAAGAC | |
| | ACCGTCATGTTGAGTTTTATACATTAGCTAGTTGTATAGCAATTGAA | |
| | CCAAAACACAGTGCTTTTCGTCTTGGTTTTGCTAAAGTTTGTTATTTA | |
| | GGTATAGTTTTAGATGATATTTATGACACATTTGGTAAAATGAAAGA | |
| | ATTAGAACTTTTTACTGCAGCAATCAAACGTTGGGACCCTTCTACTA | |
| | CAGAATGCTTACCTGAATACATGAAAGGTGTTTATATGGCTTTTTAC | |
| | AATTGTGTTAATGAATTAGCACTTCAAGCAGAGAAAACACAAGGTC | |
| | GTGATATGTTAAACTATGCACGTAAAGCATGGGAAGCTCTTTTTGAT | |
| | GCATTTTTAGAAGAAGCAAAATGGATCTCTTCTGGCTATTTACCAAC | |
| | ATTCGAAGAATACTTAGAAAATGGTAAAGTATCTTTTGGTTATCGTG | |
| | CTGCTACATTACAACCAATTTTAACATTAGATATTCCTTTACCTTTAC | |
| | ATATTTTACAACAGATTGATTTTCCAAGTCGTTTTAATGATTTAGCTT | |
| | CATCTATTTTACGTTTAAGAGGTGATATCTGTGGTTACCAAGCTGAA | |
| | CGTAGTCGTGGTGAAGAAGCATCATCAATTTCATGTTATATGAAAGA | |
| | TAATCCAGGTTCTACTGAAGAAGATGCATTATCTCACATTAATGCAA | |
| | TGATCTCAGACAATATTAACGAATTAAACTGGGAACTTTTAAAACCA | |
| | AATTCAAATGTACCAATTTCATCAAAAAAACATGCATTTGACATTCT | |
| | TCGTGCTTTCTATCACTTATACAAATATCGTGATGGCTTCTCTATCGC | |
| | AAAAATTGAAACTAAAAATCTTGTAATGCGTACAGTTTTAGAACCTG | |
| | TACCAATGGGTACCGGTGAAAACTTATACTTTCAGGGTTCTGGTGGA | |
| | GGTGGTTCAGACTATAAAGATGATGATGATAAAGGAACCGGTTAAT | |
| | CTAGACTCGAG | |
| 168 | CATATGGTACCAACAAGTGTATCAGTAGAATCAGGAACAGTATCTT | Bisabolene |
| | GTTTATCATCAAACAACTTAATTAGACGTACAGCTAATCCACATCCT | (P. abies) |
| | AACATTTGGGGATATGATTTTGTTCACTCACTTAAATCACCATATAC | |
| | ACACGACTCATCATATCGTGAACGTGCTGAGACTTTAATTTCAGAAA | |
| | TAAAAGTTATGCTTGGAGGTGGTGAATTAATGATGACTCCATCAGCT | |
| | TATGATACAGCATGGGTAGCTCGTGTTCCATCAATTGACGGTAGTGC | |
| | TTGTCCACAATTTCCACAAACTGTTGAATGGATTCTTAAAAACCAAT | |
| | TAAAAGATGGTAGTTGGGGAACTGAATCTCACTTCTTACTTAGTGAC | |
| | AGATTATTAGCTACATTAAGTTGTGTATTAGCATTATTAAAATGGAA | |
| | AGTAGCTGATGTTCAAGTAGAGCAAGGTATTGAGTTTATCAAACGTA | |
| | ATTTACAAGCTATTAAAGACGAACGTGATCAAGACAGTTTAGTAACT | |
| | GATTTCGAGATTATTTTCCCATCACTTTTAAAAGAGGCTCAATCTTTA | |
| | AACTTAGGCTTACCTTATGATTTACCATATATTAGATTATTACAAAC | |
| | AAAACGTCAAGAACGTCTTGCTAACTTAAGTATGGATAAAATTCAC | |
| | GGTGGTACTTTATTATCATCTTTAGAGGGCATTCAAGATATAGTTGA | |
| | ATGGGAAACAATTATGGATGTACAATCTCAAGATGGTTCTTTCTTAT | |
| | CATCACCAGCTTCTACAGCATGTGTATTCATGCATACAGGAGATATG | |
| | AAATGTTTAGATTTCTTAAACAACGTATTAACTAAATTTGGTAGTAG | |
| | TGTTCCTTGTTTATACCCTGTAGATTTATTAGAACGTCTTTTAATTGT | |
| | AGATAATGTAGAGCGTCTTGGTATTGACCGTCATTTTGAAAAAGAAA | |
| | TCAAAGAGGCTTTAGATTATGTTTATCGTCATTGGAACGATCGTGGT | |
| | ATTGGTTGGGGTCGTTTATCACCTATCGCAGACTTAGAAACAACAGC | |
| | TTTAGGTTTTCGTTTACTTCGTCTTCATCGTTACAATGTTTCTCCTGTA | |
| | GTATTAGACAATTTCAAAGACGCAGATGGCGAGTTCTTCTGCAGTAC | |
| | AGGTCAATTTAACAAAGATGTTGCAAGTATGTTATCTTTATACCGTG | |
| | CTTCTCAATTAGCTTTCCCTGAAGAATCAATTTTAGATGAAGCTAAA | |
| | TCATTCTCAACACAATATCTTCGTGAAGCATTAGAAAAATCAGAAAC | |
| | ATTTTCTTCTTGGAATCATCGTCAGAGTTTATCAGAAGAAATTAAAT | |
| | ATGCTTTAAAAACATCATGGCACGCTTCAGTTCCTCGTGTTGAAGCA | |
| | AAACGTTATTGTCAGGTTTACCGTCAAGACTATGCTCATTTAGCAAA | |
| | ATCAGTTTATAAACTTCCTAAAGTAAATAATGAGAAAATTCTTGAAT | |
| | TAGCAAAATTAGATTTTAACATTATTCAATCTATCCATCAAAAAGAA | |
| | ATGAAAAATGTTACATCATGGTTTCGTGATTCAGGCTTACCACTTTT | |
| | CACATTTGCTCGTGAAAGACCTTTAGAGTTTTACTTTTTAATCGCTGG | |
| | TGGAACATACGAACCTCAATACGCAAAATGTAGATTCTTATTTACAA | |
| | AAGTAGCTTGTTTACAAACTGTTTTAGACGATATGTACGATACTTAC | |
| | GGTACACCATCAGAGTTAAAATTATTTACTGAGGCAGTTCGTCGTTG | |
| | GGATTTATCATTCACAGAAAACTTACCTGATTATATGAAATTATGCT | |
| | ACAAAATTTACTATGATATTGTTCATGAAGTTGCTTGGGAAGTAGAA | |
| | AAAGAACAGGGACGTGAGCTTGTTTCATTTTTCCGTAAAGGTTGGGA | |
| | AGACTATCTTTTAGGTTATTATGAAGAAGCTGAATGGTTAGCTGCTG | |
| | AATACGTTCCTACTTTAGATGAATACATTAAAAACGGTATTACATCT | |
| | ATTGGTCAACGTATTTTACTTTTATCAGGTGTACTTATTATGGAAGGT | |
| | CAACTTTTATCACAAGAAGCTCTTGAAAAAGTAGATTATCCAGGTCG | |
| | TCGTGTTTTAACAGAATTAAACAGTTTAATTAGTCGTTTAGCAGACG | |
| | ATACTAAAACATACAAAGCAGAAAAAGCTCGTGGTGAACTTGCTAG | |
| | TAGTATTGAATGTTATATGAAAGACCACCCTGGTTGTCAAGAAGAA | |
| | GAAGCATTAAACCATATTTATGGCATTTTAGAACCAGCTGTTAAAGA | |
| | ATTAACTCGTGAGTTTCTTAAAGCAGATCACGTACCATTCCCTTGCA | |
| | AAAAAATGTTATTTGATGAAACAAGAGTTACAATGGTAATTTTCAAA | |
| | GATGGTGATGGTTTCGGTATTTCTAAATTAGAAGTAAAAGACCACAT | |
| | AAAAGAATGTTTAATTGAGCCATTACCACTTGGTACCGGTGAAAATC | |
| | TTTATTTTCAAGGTAGTGGTGGTGGCGGTTCTGACTACAAAGATGAC | |
| | GACGATAAAGGAACCGGTTAATCTAGACTCGAG | |
| 169 | CATATGGTACCAGGTTCTGAAGTAAATAGACCTTTAGCAGACTTTCC | Sesquiterpene |
| | AGCAAACATTTGGGAAGACCCATTAACTTCTTTCTCAAAATCTGATC | (A. thalania) |
| | TTGGTACAGAAACATTTAAAGAGAAACATAGTACTTTAAAAGAAGC | |
| | TGTTAAAGAGGCATTTATGAGTTCTAAAGCTAATCCAATCGAAAATA | |
| | TCAAATTCATAGATGCATTATGCCGTTTAGGAGTATCTTATCACTTTG | |
| | AAAAAGATATTGTAGAACAATTAGATAAATCATTTGATTGCTTAGAT | |
| | TTTCCACAAATGGTACGTCAAGAAGGTTGCGATTTATATACAGTTGG | |
| | TATTATCTTTCAAGTTTTTAGACAATTTGGTTTCAAATTAAGTGCTGA | |
| | TGTTTTTGAAAAATTCAAAGATGAAAATGGTAAATTCAAAGGTCACT | |
| | TAGTAACTGATGCTTATGGTATGTTATCATTATACGAAGCTGCACAA | |
| | TGGGGTACTCACGGTGAAGACATCATTGACGAAGCTCTTGCTTTTTC | |
| | TCGTAGTCACTTAGAAGAAATATCTAGTCGTAGTTCACCACACTTAG | |
| | CAATTCGTATTAAAAACGCTTTAAAACATCCATATCATAAAGGTATT | |
| | TCACGTATTGAAACACGTCAATACATTAGTTACTATGAAGAAGAAG | |
| | AATCTTGTGATCCAACATTATTAGAGTTCGCTAAAATTGACTTTAAC | |
| | TTATTACAAATTTTACACCGTGAAGAGTTAGCTTGTGTAACTCGTTG | |
| | GCATCATGAAATGGAATTTAAAAGTAAAGTAACTTACACACGTCAT | |
| | CGTATTACAGAAGCATATTTATGGAGTCTTGGAACATATTTTGAACC | |
| | ACAATACAGTCAAGCTCGTGTAATAACTACAATGGCATTAATCTTAT | |
| | TTACTGCTTTAGACGACATGTACGATGCTTACGGTACTATGGAGGAG | |
| | TTAGAGTTATTCACAGATGCTATGGACGAATGGTTACCAGTTGTTCC | |
| | AGATGAAATTCCTATTCCAGATTCAATGAAATTCATTTACAATGTTA | |
| | CAGTTGAATTTTACGATAAATTAGACGAAGAATTAGAAAAAGAAGG | |
| | TCGTTCTGGTTGTGGTTTCCATCTTAAAAAAAGTTTACAAAAAACAG | |
| | CTAATGGATATATGCAAGAAGCAAAATGGCTTAAAAAAGATTACAT | |
| | TGCTACATTTGATGAGTATAAAGAAAATGCTATTTTATCTTCAGGTT | |
| | ATTATGCATTAATTGCAATGACATTTGTTCGTATGACTGATGTTGCTA | |
| | AATTAGATGCTTTTGAATGGTTAAGTAGTCACCCAAAAATTCGTGTA | |
| | GCAAGTGAAATCATTTCACGTTTTACAGACGATATTTCAAGTTATGA | |
| | ATTTGAACACAAACGTGAACACGTTGCTACAGGTATTGATTGTTATA | |
| | TGCAACAATTCGGAGTTAGTAAAGAACGTGCTGTTGAAGTTATGGG | |
| | CAATATAGTTTCTGATGCATGGAAAGACTTAAATCAAGAACTTATGC | |
| | GTCCTCATGTTTTCCCATTTCCACTTCTTATGCGTGTTTTAAATCTTTC | |
| | AAGAGTAATTGATGTATTTTATCGTTACCAAGATGCATATACTAATC | |
| | CAAAATTACTTAAAGAGCACATTGTTTCTTTACTTATTGAAACTATTC | |
| | CAATTGGTACCGGTGAAAACTTATACTTTCAAGGTAGTGGTGGAGGT | |
| | GGTTCTGATTATAAAGACGACGATGACAAAGGAACCGGTTAATCTA | |
| | GACTCGAG | |
| 170 | CATATGGTACCAGAGGCAATTAGAGTATTTGGCTTAAAACTTGGTTC | Sesquiterpene |
| | AAAATTATCTATTCACTCACAAACAAATGCTTTTCCTGCATTCAAAT | (A. thalania) |
| | TATCTCGTTTTCCATTAACATCTTTCCCTGGTAAACATGCTCACTTAG | |
| | ATCCATTAAAAGCAACAACTCATCCATTAGCTTTTGATGGTGAAGAA | |
| | AATAACCGTGAGTTTAAAAACTTAGGTCCAAGTGAGTGGGGCCATC | |
| | AATTTCTTTCTGCTCATGTAGATTTATCTGAAATGGATGCATTAGAA | |
| | CGTGAAATTGAAGCTCTTAAACCAAAAGTACGTGATATGTTAATATC | |
| | AAGTGAAAGTTCAAAAAAAAAAATCTTATTTCTTTATCTTTTAGTAT | |
| | CATTAGGATTAGCTTATCACTTTGAAGATGAAATTAAAGAAAGTTTA | |
| | GAGGATGGATTACAGAAAATTGAGGAAATGATGGCTTCAGAAGATG | |
| | ATCTTCGTTTTAAAGGCGATAATGGTAAATTCAAAGAATGTTTAGCA | |
| | AAAGATGCTAAAGGTATTTTATCTCTTTATGAGGCTGCTCACATGGG | |
| | TACAACAACTGATTATATTCTTGATGAGGCTTTATCATTTACTTTAAC | |
| | ATATATGGAATCATTAGCAGCTTCAGGAACATGTAAAATCAACTTAT | |
| | CACGTCGTATTAGAAAAGCATTAGATCAACCTCAACACAAAAATAT | |
| | GGAAATAATTGTAGCAATGAAATACATTCAATTTTATGAAGAAGAG | |
| | GAAGATTGCGATAAAACTTTACTTAAATTTGCTAAACTTAACTTTAA | |
| | ATTCTTACAATTACACTATTTACAAGAACTTAAAATCTTATCTAAAT | |
| | GGTATAAAGACCAAGACTTTAAATCAAAATTACCTCCATATTTCCGT | |
| | GACCGTCTTGTAGAATGTCATTTTGCATCATTAACATGTTTTGAGCCT | |
| | AAATATGCTCGTGCACGTATTTTCTTATCTAAAATCTTCACTGTTCAA | |
| | ATTTTCATTGACGATACTTGTGACCGTTACGCATCATTAGGTGAAGT | |
| | TGAGTCATTAGCTGACACTATCGAACGTTGGGACCCTGATGATCATG | |
| | CTATGGACGGATTACCTGATTATCTTAAATCAGTAGTTAAATTTGTA | |
| | TTCAATACATTTCAAGAATTTGAACGTAAATGTAAACGTTCACTTCG | |
| | TATTAACTTACAAGTAGCAAAATGGGTTAAAGCTGGTCACTTACCAT | |
| | CTTTTGATGAGTATCTTGATGTAGCTGGTTTAGAATTAGCTATTTCAT | |
| | TCACTTTCGCTGGTATCTTAATGGGCATGGAAAATGTTTGTAAACCT | |
| | GAAGCATACGAATGGTTAAAATCTCGTGACAAACTTGTTCGTGGTGT | |
| | AATCACAAAAGTTCGTTTACTTAATGATATTTTTGGCTATGAAGATG | |
| | ATATGCGTCGTGGTTATGTAACAAATTCAATAAACTGCTACAAAAAA | |
| | CAATATGGAGTAACAGAGGAAGAAGCTATTCGTAAATTACATCAAA | |
| | TCGTTGCTGATGGAGAGAAAATGATGAATGAAGAGTTCTTAAAACC | |
| | TATTAATGTACCATATCAGGTTCCTAAAGTAGTTATTTTAGACACTTT | |
| | ACGTGCAGCTAATGTTTCATACGAAAAAGATGACGAATTTACACGTC | |
| | CAGGCGAACACCTTAAAAACTGCATTACATCTATTTACTTCGATTTA | |
| | GGTACCGGTGAAAACTTATACTTTCAAGGTAGTGGTGGCGGTGGTA | |
| | GTGATTACAAAGATGATGATGATAAAGGAACCGGTTAATCTAGACT | |
| | CGAG | |
| 171 | CATATGGTACCAACTACAACATTATCATCTAACCTTAACTCACAATT | GPP |
| | CATGCAGGTTTACGAGACTCTTAAATCAGAACTTATTCATGACCCAT | Chimera |
| | TATTTGAGTTCGATGACGATTCAAGACAATGGGTAGAACGTATGATT | |
| | GATTATACTGTACCAGGTGGTAAAATGGTTCGTGGTTATAGTGTAGT | |
| | AGATAGTTATCAATTACTTAAAGGTGAAGAACTTACAGAAGAAGAG | |
| | GCATTTTTAGCTTGTGCACTTGGTTGGTGTACAGAATGGTTTCAAGC | |
| | ATTCATTCTTTTACATGATGATATGATGGATGGTAGTCACACAAGAC | |
| | GTGGTCAACCATGTTGGTTTCGTTTACCTGAGGTTGGTGCTGTTGCTA | |
| | TTAATGATGGTGTTTTACTTCGTAATCACGTTCACCGTATTCTTAAAA | |
| | AACATTTTCAAGGTAAAGCATATTATGTTCATTTAGTTGATTTATTCA | |
| | ATGAAACTGAATTTCAAACAATTAGTGGACAAATGATCGACTTAATT | |
| | ACAACATTAGTTGGTGAAAAAGACTTATCTAAATATTCATTAAGTAT | |
| | TCATCGTCGTATCGTTCAATACAAAACAGCATACTACTCATTTTACTT | |
| | ACCAGTTGCTTGTGCTTTACTTATGTTTGGTGAGGATCTTGATAAAC | |
| | ATGTAGAAGTTAAAAATGTTCTTGTTGAAATGGGTACATATTTTCAA | |
| | GTTCAAGATGATTATTTAGATTGTTTTGGTGCTCCAGAAGTTATTGG | |
| | CAAAATTGGTACTGATATTGAAGACTTTAAATGTTCATGGTTAGTAG | |
| | TTAAAGCATTAGAATTAGCAAATGAAGAACAGAAAAAAACTTTACA | |
| | CGAAAATTATGGAAAAAAAGATCCAGCATCAGTTGCTAAAGTTAAA | |
| | GAAGTATACCACACACTTAATTTACAAGCTGTTTTCGAAGATTATGA | |
| | AGCAACATCATACAAAAAACTTATTACTTCTATTGAAAATCACCCAT | |
| | CTAAAGCTGTTCAAGCTGTTTTAAAATCTTTCTTAGGCAAAATATAC | |
| | AAACGTCAAAAAGGTACCGGTGAAAACTTATACTTTCAAGGTTCTG | |
| | GTGGCGGTGGAAGTGATTACAAAGATGATGACGATAAAGGAACCGG | |
| | TTAATCTAGACTCGAG | |
| 172 | CATATGGTACCAAGTCAACCTTACTGGGCTGCAATTGAAGCAGACAT | GPPS- |
| | TGAAAGATATTTAAAAAAATCAATTACAATTCGTCCACCAGAAACT | LSU + SSU |
| | GTATTTGGTCCTATGCACCATTTAACATTTGCTGCTCCTGCTACTGCA | fusion |
| | GCTAGTACATTATGCCTTGCTGCTTGTGAATTAGTTGGCGGTGATCG | |
| | TAGTCAAGCTATGGCAGCTGCTGCTGCTATCCATTTAGTTCATGCAG | |
| | CTGCTTACGTTCACGAACATCTTCCTTTAACAGATGGATCACGTCCT | |
| | GTAAGTAAACCTGCTATTCAACATAAATATGGTCCAAACGTTGAACT | |
| | TTTAACAGGTGATGGTATCGTTCCTTTCGGTTTTGAGTTATTAGCAGG | |
| | TTCAGTAGATCCAGCACGTACTGATGACCCTGATCGTATTTTACGTG | |
| | TAATTATTGAAATTTCTCGTGCTGGTGGACCAGAAGGCATGATTTCT | |
| | GGTTTACACCGTGAGGAAGAAATCGTAGATGGTAACACATCATTAG | |
| | ACTTTATAGAATATGTATGCAAAAAAAAATACGGTGAAATGCACGC | |
| | ATGTGGTGCAGCTTGCGGAGCTATTTTAGGTGGAGCTGCTGAAGAA | |
| | GAAATTCAAAAACTTCGTAACTTTGGTCTTTATCAAGGCACATTACG | |
| | TGGTATGATGGAAATGAAAAATAGTCATCAGTTAATTGACGAAAAT | |
| | ATCATTGGAAAACTTAAAGAACTTGCTCTTGAAGAATTAGGTGGATT | |
| | CCACGGTAAAAACGCTGAATTAATGAGTTCTTTAGTTGCTGAACCTA | |
| | GTTTATATGCAGCTTCATCAAATAACTTAGGTATCGAAGGTCGTTTT | |
| | GACTTTGACGGTTACATGCTTCGTAAAGCAAAATCTGTAAATAAAGC | |
| | ATTAGAAGCTGCTGTTCAAATGAAAGAACCACTTAAAATTCACGAA | |
| | TCAATGCGTTATTCATTATTAGCTGGTGGTAAACGTGTTCGTCCAAT | |
| | GTTATGTATTGCAGCTTGTGAACTTGTTGGTGGTGACGAATCTACAG | |
| | CAATGCCTGCAGCATGTGCTGTTGAAATGATTCACACAATGTCTTTA | |
| | ATGCATGATGACCTTCCATGTATGGATAACGATGACTTACGTCGTGG | |
| | TAAACCTACAAACCACATGGCTTTTGGTGAGTCTGTAGCTGTTCTTG | |
| | CTGGTGATGCATTACTTAGTTTTGCTTTTGAACATGTTGCTGCTGCAA | |
| | CAAAAGGCGCACCACCTGAACGTATCGTACGTGTATTAGGTGAATT | |
| | AGCTGTTAGTATTGGTTCAGAAGGACTTGTAGCAGGTCAAGTTGTAG | |
| | ACGTTTGTTCTGAAGGCATGGCTGAAGTAGGATTAGATCATCTTGAA | |
| | TTTATTCACCATCATAAAACTGCTGCATTATTACAAGGTTCAGTTGTT | |
| | TTAGGTGCAATATTAGGAGGCGGTAAAGAAGAAGAAGTAGCTAAAC | |
| | TTCGTAAATTTGCTAACTGTATTGGTTTACTTTTCCAAGTTGTTGATG | |
| | ATATTTTAGATGTTACTAAAAGTAGTAAAGAGTTAGGTAAAACTGCA | |
| | GGTAAAGACTTAGTAGCTGATAAAACTACATATCCTAAACTTATAGG | |
| | CGTTGAAAAATCAAAAGAATTTGCTGACCGTTTAAATCGTGAAGCA | |
| | CAAGAACAATTATTACATTTTCATCCTCACCGTGCTGCTCCATTAATC | |
| | GCTTTAGCTAACTACATCGCTTACCGTGATAATGGTACCGGTGAAAA | |
| | CTTATACTTCCAGGGTAGTGGTGGTGGCGGATCAGATTATAAAGATG | |
| | ACGATGATAAAGGAACCGGTTAATCTAGACTCGAG | |
| 173 | CATATGGTACCAGTAACAGCAGCACGTGCAACACCAAAATTAAGTA | Geranyl- |
| | ATAGAAAATTACGTGTTGCTGTAATTGGAGGCGGTCCAGCAGGAGG | geranyl |
| | TGCAGCTGCTGAAACATTAGCACAAGGAGGTATTGAAACAATTCTT | reductase |
| | ATCGAACGTAAAATGGATAATTGTAAACCATGTGGTGGTGCTATTCC | (A. thalania) |
| | ATTATGTATGGTAGGAGAGTTCAATTTACCTTTAGACATTATTGACC | |
| | GTCGTGTAACAAAAATGAAAATGATCTCTCCTTCAAACATTGCAGTT | |
| | GATATCGGTCGTACACTTAAAGAACACGAATATATTGGTATGGTTCG | |
| | TCGTGAGGTACTTGATGCTTATCTTCGTGAACGTGCAGAAAAATCAG | |
| | GTGCTACTGTTATTAACGGTTTATTCTTAAAAATGGATCACCCAGAA | |
| | AATTGGGATTCACCATATACACTTCACTACACAGAGTATGATGGAAA | |
| | AACAGGTGCTACAGGAACTAAAAAAACTATGGAAGTAGATGCTGTT | |
| | ATTGGTGCTGATGGTGCTAATTCTCGTGTTGCAAAAAGTATTGACGC | |
| | AGGTGATTATGATTATGCTATTGCATTTCAAGAACGTATTCGTATAC | |
| | CTGATGAGAAAATGACTTATTATGAGGACTTAGCTGAGATGTATGTA | |
| | GGTGATGATGTATCACCAGACTTCTACGGTTGGGTATTCCCAAAATG | |
| | TGATCATGTAGCTGTTGGTACAGGTACTGTAACACATAAAGGTGATA | |
| | TCAAAAAATTCCAGTTAGCTACACGTAATCGTGCTAAAGATAAAATT | |
| | CTTGGTGGCAAAATAATCCGTGTAGAGGCTCATCCTATTCCAGAGCA | |
| | TCCTAGACCACGTCGTTTATCAAAACGTGTTGCATTAGTAGGCGACG | |
| | CAGCAGGTTACGTTACTAAATGTTCAGGAGAAGGAATTTACTTCGCA | |
| | GCTAAATCTGGTCGTATGTGTGCTGAAGCTATCGTTGAAGGTTCACA | |
| | AAATGGCAAAAAAATGATAGATGAAGGCGATTTAAGAAAATACTTA | |
| | GAAAAATGGGATAAAACTTACTTACCAACTTATCGTGTTTTAGATGT | |
| | ACTTCAAAAAGTTTTCTATCGTTCTAACCCAGCTCGTGAGGCTTTTGT | |
| | TGAAATGTGTAACGATGAGTATGTACAGAAAATGACATTTGATTCTT | |
| | ACCTTTATAAACGTGTAGCTCCTGGTAGTCCATTAGAAGATATCAAA | |
| | TTAGCTGTAAATACTATTGGTTCACTTGTTCGTGCTAACGCATTACGT | |
| | CGTGAAATTGAGAAATTATCAGTAGGTACCGGTGAGAATCTTTACTT | |
| | TCAAGGATCAGGTGGTGGTGGTTCTGATTATAAAGATGACGATGAT | |
| | AAAGGAACCGGTTAATCTAGACTCGAG | |
| 174 | CATATGGTACCAGTAGCTGTTATTGGTGGTGGTCCAAGTGGCGCTTG | Geranylgeranyl |
| | TGCAGCAGAAACTTTAGCAAAAGGTGGTGTAGAAACTTTCTTACTTG | reductase |
| | AGCGTAAATTAGATAATTGTAAACCTTGTGGAGGTGCAATTCCATTA | (C. reinhardtii) |
| | TGTATGGTTGAAGAATTTGATTTACCAATGGAAATAATTGACCGTCG | |
| | TGTTACTAAAATGAAAATGATATCACCTTCAAACCGTGAAGTTGATG | |
| | TTGGAAAAACTTTATCAGAAACTGAATGGATCGGTATGTGTCGTCGT | |
| | GAAGTATTTGACGATTACTTAAGAAACCGTGCACAGAAATTAGGTG | |
| | CTAATATTGTTAACGGTTTATTCATGCGTTCAGAACAACAATCTGCA | |
| | GAGGGTCCATTCACAATTCACTATAATTCTTATGAAGACGGTAGTAA | |
| | AATGGGAAAACCTGCTACTTTAGAAGTTGATATGATAATTGGTGCAG | |
| | ATGGAGCAAATTCTCGTATTGCAAAAGAGATAGATGCAGGTGAATA | |
| | CGACTACGCTATAGCTTTTCAAGAACGTATTCGTATTCCTGATGATA | |
| | AAATGAAATATTACGAAAACCTTGCTGAAATGTATGTAGGTGATGA | |
| | CGTATCTCCTGATTTCTATGGTTGGGTTTTTCCTAAATATGATCACGT | |
| | TGCTGTTGGTACAGGTACTGTTGTAAACAAAACAGCTATTAAACAAT | |
| | ATCAACAGGCAACACGTGACAGATCAAAAGTTAAAACAGAAGGTGG | |
| | CAAAATTATACGTGTTGAAGCACACCCAATTCCAGAACATCCACGTC | |
| | CACGTCGTTGTAAAGGTCGTGTTGCATTAGTAGGCGACGCAGCTGGT | |
| | TATGTTACAAAATGTTCTGGCGAGGGCATTTACTTTGCTGCTAAATC | |
| | TGGTAGAATGGCTGCTGAAGCTATTGTAGAAGGTTCTGCTAACGGTA | |
| | CAAAAATGTGTGGTGAGGATGCAATTCGTGTTTATTTAGATAAATGG | |
| | GATCGTAAATATTGGACAACATACAAAGTATTAGACATTTTACAAA | |
| | AAGTATTTTATCGTAGTAATCCAGCACGTGAAGCATTTGTTGAATTA | |
| | TGTGAAGATAGTTATGTACAGAAAATGACATTTGATTCATACTTATA | |
| | TAAAACTGTTGTTCCAGGAAACCCATTAGACGACGTAAAATTACTTG | |
| | TTCGTACAGTATCTTCTATTTTACGTTCAAATGCTTTACGTTCTGTTA | |
| | ATTCTAAATCTGTAAATGTTTCTTTCGGCTCTAAAGCAAATGAGGAA | |
| | CGTGTTATGGCTGCAGGTACCGGTGAAAATCTTTATTTTCAAGGTTC | |
| | AGGAGGTGGTGGTTCAGATTATAAAGATGATGATGACAAAGGAACC | |
| | GGTTAATCTAGACTCGAG | |
| 175 | CATATGGTACCAGCAATGGCAGTACCATTAGATGTAGTAATTACATA | Chlorophyllidohydrolase |
| | TCCTTCTTCAGGTGCTGCTGCTTATCCAGTACTTGTTATGTATAACGG | (C. reinhardtii) |
| | TTTCCAAGCTAAAGCTCCATGGTATCGTGGTATTGTAGATCATGTTT | |
| | CTAGTTGGGGTTACACAGTTGTTCAATATACAAATGGTGGCTTATTT | |
| | CCTATTGTTGTAGATCGTGTTGAGTTAACTTATTTAGAGCCATTATTA | |
| | ACTTGGTTAGAAACACAAAGTGCTGATGCTAAATCTCCTTTATACGG | |
| | TCGTGCAGATGTTTCTCGTTTAGGTACAATGGGTCATTCACGTGGTG | |
| | GTAAATTAGCAGCTTTACAATTTGCTGGACGTACAGATGTAAGTGGT | |
| | TGTGTATTATTTGACCCTGTAGATGGAAGTCCAATGACACCAGAATC | |
| | TGCTGATTATCCTTCAGCTACAAAAGCATTAGCAGCAGCTGGTCGTT | |
| | CTGCTGGCTTAGTAGGTGCAGCTATTACAGGTTCATGTAATCCAGTA | |
| | GGTCAAAATTACCCAAAATTCTGGGGTGCTTTAGCTCCTGGTTCTTG | |
| | GCAAATGGTATTATCACAAGCTGGTCACATGCAATTTGCTCGTACTG | |
| | GTAATCCATTCTTAGATTGGTCATTAGACCGTTTATGTGGTCGTGGT | |
| | ACAATGATGAGTTCAGATGTTATTACATATAGTGCAGCATTTACTGT | |
| | TGCTTGGTTTGAAGGTATTTTTCGTCCTGCTCAAAGTCAAATGGGTA | |
| | TTTCTAATTTCAAAACTTGGGCTAATACTCAAGTTGCAGCTCGTAGT | |
| | ATCACTTTTGATATTAAACCTATGCAATCTCCTCAGGGTACCGGTGA | |
| | AAACCTTTACTTTCAAGGTAGTGGTGGTGGAGGAAGTGATTATAAA | |
| | GATGATGATGACAAAGGAACCGGTTAATCTAGACTCGAG | |
| 176 | CATATGGTACCAGCACCACCAAAACCAGTTCGTATAACTTGTCCAAC | Chlorophyllido |
| | AGTAGCTGGCACTTATCCTGTTGTTTTATTCTTTCACGGTTTTTATCTT | hydrolase |
| | CGTAACTATTTCTATTCAGATGTTTTAAATCATATTGCTAGTCATGGT | (A. thalania) |
| | TACATCTTAGTTGCACCACAATTATGTAAACTTTTACCTCCAGGTGG | |
| | CCAAGTAGAAGTTGATGACGCTGGTTCAGTTATTAACTGGGCTTCAG | |
| | AGAATCTTAAAGCACACCTTCCAACTTCTGTTAATGCTAATGGTAAA | |
| | TATACATCTTTAGTTGGACATTCACGTGGTGGCAAAACAGCTTTCGC | |
| | AGTTGCATTAGGTCACGCAGCTACATTAGATCCATCAATTACATTTT | |
| | CAGCATTAATTGGTATTGATCCAGTAGCAGGAACTAACAAATACATT | |
| | CGTACAGATCCACACATCTTAACTTATAAACCTGAATCATTTGAATT | |
| | AGATATTCCTGTAGCTGTTGTAGGCACTGGTCTTGGTCCAAAATGGA | |
| | ATAACGTAATGCCTCCATGCGCACCTACAGATTTAAACCACGAAGA | |
| | ATTTTACAAAGAATGTAAAGCTACTAAAGCTCACTTTGTTGCTGCTG | |
| | ATTATGGTCACATGGACATGTTAGACGACGATCTTCCAGGTTTTGTA | |
| | GGCTTCATGGCTGGTTGTATGTGTAAAAATGGTCAACGTAAAAAATC | |
| | AGAAATGCGTTCTTTTGTAGGTGGTATAGTTGTAGCATTCTTAAAAT | |
| | ATTCTTTATGGGGTGAAAAAGCTGAAATAAGATTAATTGTTAAAGAT | |
| | CCTAGTGTATCTCCTGCTAAATTAGACCCATCACCAGAATTAGAAGA | |
| | AGCATCAGGTATTTTTGTTGGTACCGGTGAAAATCTTTATTTTCAAG | |
| | GTTCAGGTGGAGGTGGTTCTGATTATAAAGATGATGATGACAAAGG | |
| | AACCGGTTAATCTAGACTCGAG | |
| 177 | CATATGGTACCAGCTACACCAGTTGAAGAAGGTGATTATCCAGTTGT | Chlorophyllidohydrolase |
| | AATGTTATTACATGGCTACCTTTTATATAATTCATTTTATTCACAATT | (A. thalania) |
| | AATGTTACATGTATCATCTCACGGTTTCATCTTAATTGCTCCACAATT | |
| | ATACTCAATTGCTGGTCCTGATACTATGGATGAAATTAAAAGTACTG | |
| | CTGAGATTATGGACTGGTTATCAGTTGGTTTAAATCACTTTTTACCA | |
| | GCTCAAGTTACACCTAATTTATCTAAATTTGCATTATCTGGTCATAGT | |
| | CGTGGTGGTAAAACTGCTTTTGCTGTAGCATTAAAAAAATTTGGTTA | |
| | TTCTTCAAACTTAAAAATTAGTACTTTAATTGGTATTGATCCAGTAG | |
| | ACGGAACAGGTAAAGGTAAACAAACTCCACCTCCTGTTTTAGCATAT | |
| | TTACCTAATAGTTTTGACTTAGACAAAACACCAATTTTAGTAATTGG | |
| | TTCAGGTTTAGGTGAAACTGCACGTAATCCTTTATTTCCTCCATGTGC | |
| | TCCTCCAGGTGTTAACCACCGTGAGTTTTTCCGTGAATGTCAAGGTC | |
| | CAGCATGGCACTTTGTTGCTAAAGATTATGGTCATTTAGACATGCTT | |
| | GATGATGATACAAAAGGTATTCGTGGCAAATCTAGTTACTGTTTATG | |
| | CAAAAATGGTGAAGAACGTCGTCCAATGCGTCGTTTCGTTGGTGGTT | |
| | TAGTTGTTAGTTTTCTTAAAGCATATCTTGAAGGTGATGATCGTGAA | |
| | TTAGTAAAAATCAAAGATGGTTGTCATGAAGATGTACCTGTTGAAAT | |
| | TCAAGAATTTGAAGTAATTATGGGTACCGGTGAAAATCTTTACTTTC | |
| | AAGGTTCAGGCGGTGGAGGTTCAGATTATAAAGATGATGATGACAA | |
| | AGGAACCGGTTAATCTAGACTCGAG | |
| 178 | CATATGGTACCAAGTCACAAAAAAAAAAACGTAATCTTCTTCGTAA | Phosphatase |
| | CTGATGGTATGGGTCCTGCTTCTCTTTCAATGGCTCGTTCATTTAATC | (S. cerevisiae) |
| | AACACGTTAATGATTTACCAATTGATGATATTTTAACATTAGATGAA | |
| | CATTTTATTGGAAGTTCAAGAACACGTTCATCAGATTCACTTGTAAC | |
| | TGACTCAGCTGCTGGAGCTACAGCTTTTGCTTGTGCACTTAAATCAT | |
| | ACAATGGTGCTATAGGTGTAGATCCACACCATCGTCCATGTGGAACT | |
| | GTTTTAGAAGCTGCTAAATTAGCAGGTTATTTAACAGGATTAGTAGT | |
| | TACTACACGTATTACTGATGCTACACCAGCTAGTTTCTCAAGTCACG | |
| | TAGATTATCGTTGGCAAGAAGATTTAATTGCAACACACCAATTAGGT | |
| | GAATATCCTTTAGGACGTGTTGTTGATCTTCTTATGGGTGGTGGTCGT | |
| | TCTCACTTTTATCCTCAAGGTGAAAAAGCTAGTCCATACGGTCACCA | |
| | CGGTGCACGTAAAGATGGTCGTGATTTAATCGATGAAGCTCAAAGT | |
| | AATGGCTGGCAGTATGTAGGAGATCGTAAAAATTTTGATTCTTTACT | |
| | TAAATCACATGGTGAAAATGTTACTTTACCATTTTTAGGTTTATTTGC | |
| | TGACAACGATATCCCATTTGAAATTGATCGTGATGAAAAAGAATATC | |
| | CTAGTTTAAAAGAACAAGTAAAAGTAGCATTAGGTGCTTTAGAAAA | |
| | AGCAAGTAACGAAGATAAAGATAGTAATGGTTTCTTTTTAATGGTAG | |
| | AAGGTTCTCGTATTGATCATGCTGGCCATCAAAACGATCCTGCATCT | |
| | CAAGTACGTGAAGTATTAGCATTTGATGAGGCTTTTCAATATGTATT | |
| | AGAATTTGCAGAAAACAGTGATACAGAAACAGTATTAGTAAGTACA | |
| | TCAGATCATGAAACAGGTGGTTTAGTTACTTCAAGACAAGTAACAG | |
| | CATCATACCCACAATATGTATGGTATCCTCAAGTATTAGCTAACGCT | |
| | ACACATAGTGGAGAGTTTCTTAAACGTAAATTAGTTGATTTCGTTCA | |
| | TGAACACAAAGGCGCATCATCAAAAATAGAAAACTTCATAAAACAC | |
| | GAAATTCTTGAAAAAGATTTAGGTATTTATGATTATACAGATTCTGA | |
| | CTTAGAAACACTTATTCATTTAGATGATAACGCTAATGCAATTCAAG | |
| | ATAAACTTAATGATATGGTAAGTTTTAGAGCTCAAATTGGTTGGACA | |
| | ACACATGGTCATTCAGCAGTTGATGTAAACATATATGCTTACGCAAA | |
| | CAAAAAAGCTACATGGTCTTATGTTCTTAATAACTTACAAGGTAATC | |
| | ACGAAAACACAGAAGTTGGTCAATTCTTAGAGAATTTCTTAGAATTA | |
| | AACTTAAATGAAGTTACTGATTTAATCCGTGATACAAAACATACTTC | |
| | TGATTTTGACGCAACAGAAATAGCAAGTGAGGTTCAACACTATGAT | |
| | GAATATTACCACGAATTAACAAATGGTACCGGTGAAAATCTTTATTT | |
| | TCAAGGTTCTGGTGGAGGTGGCAGTGATTATAAAGATGATGATGAC | |
| | AAAGGAACCGGTTAATCTAGACTCGAG | |
| 179 | CATATGGTACCACACAAGTTCACAGGTGTTAACGCTAAATTCCAGCA | FPP A118W |
| | ACCAGCATTAAGAAATTTATCTCCAGTGGTAGTTGAGCGCGAACGTG | (G. gallus) |
| | AGGAATTTGTAGGATTCTTTCCACAAATTGTTCGTGACTTAACTGAA | |
| | GATGGTATTGGTCATCCAGAAGTAGGTGACGCTGTAGCTCGTCTTAA | |
| | AGAAGTATTACAATACAACGCACCTGGTGGTAAATGCAATAGAGGT | |
| | TTAACAGTTGTTGCAGCTTACCGTGAACTTTCTGGACCAGGTCAAAA | |
| | AGACGCTGAAAGTCTTCGTTGTGCTTTAGCAGTAGGATGGTGTATTG | |
| | AATTATTCCAAGCCTTTTTCTTAGTTTGGGACGATATAATGGACCAG | |
| | TCATTAACTAGACGTGGTCAATTATGTTGGTACAAGAAAGAAGGTGT | |
| | TGGTTTAGATGCAATAAATGATTCTTTTCTTTTAGAAAGCTCTGTGTA | |
| | TCGCGTTCTTAAAAAGTATTGCCGTCAACGTCCATATTATGTACATTT | |
| | ATTAGAGCTTTTTCTTCAAACAGCTTACCAAACAGAATTAGGACAAA | |
| | TGTTAGATTTAATCACTGCTCCTGTATCTAAGGTAGATTTAAGCCATT | |
| | TCTCAGAAGAACGTTACAAAGCTATTGTTAAGTATAAAACTGCTTTC | |
| | TATTCATTCTATTTACCAGTTGCAGCAGCTATGTATATGGTTGGTATA | |
| | GATTCTAAAGAAGAACATGAAAACGCAAAAGCTATTTTACTTGAGA | |
| | TGGGTGAATACTTCCAAATTCAAGATGATTATTTAGATTGTTTTGGC | |
| | GATCCTGCTTTAACAGGTAAAGTAGGTACTGATATTCAAGATAACAA | |
| | ATGTTCATGGTTAGTTGTGCAATGCTTACAAAGAGTAACACCAGAAC | |
| | AACGTCAACTTTTAGAAGATAATTACGGTCGTAAAGAACCAGAAAA | |
| | AGTTGCTAAAGTTAAAGAATTATATGAGGCTGTAGGTATGAGAGCC | |
| | GCCTTTCAACAATACGAAGAAAGTAGTTACCGTCGTCTTCAAGAGTT | |
| | AATTGAGAAACATTCTAATCGTTTACCAAAAGAAATTTTCTTAGGTT | |
| | TAGCTCAGAAAATATACAAACGTCAAAAAGGTACCGGTGAAAACTT | |
| | ATACTTTCAAGGCTCAGGTGGCGGTGGAAGTGATTACAAAGATGAT | |
| | GATGATAAAGGAACCGGTTAATCTAGACTCGAG | |

TABLE 7

Nucleic acids encoding exemplary isoprenoid producing
enzymes used to increase phytol production

| | | Enzyme |
|---|---|---|
| SEQ | | (synthase) |
| ID NO. | Codon-biased, Synthesized Gene Sequence | encoded |
| 180 | ATGGTACCAGCATTTGACTTCGATGGTTACATGCTTCGTAAAGCT | GPPS-LSU (M. spicata) |
| | AAATCTGTAAATAAAGCTCTTGAAGCTGCAGTACAAATGAAAGA | |
| | ACCATTAAAAATTCATGAAAGTATGCGTTATTCTTTATTAGCTGG | |
| | TGGTAAACGTGTACGTCCAATGTTATGTATTGCAGCTTGTGAATT | |
| | AGTTGGTGGTGACGAAAGTACTGCTATGCCTGCTGCTTGCGCTG | |
| | TAGAAATGATTCATACTATGAGTTTAATGCATGATGATTTACCAT | |
| | GTATGGATAATGACGATTTACGTCGTGGTAAACCAACAAACCAC | |
| | ATGGCATTTGGTGAAAGTGTAGCAGTATTAGCAGGTGATGCATT | |
| | ATTATCTTTTGCTTTTGAACATGTAGCAGCAGCAACAAAAGGTG | |
| | CTCCTCCAGAACGTATTGTTAGAGTTTTAGGTGAACTTGCAGTTT | |
| | CTATTGGTTCAGAAGGTTTAGTTGCTGGACAAGTAGTTGACGTTT | |
| | GTTCTGAAGGTATGGCTGAGGTTGGTTTAGATCATTTAGAATTTA | |
| | TTCATCACCACAAAACTGCTGCTTTATTACAAGGTTCTGTAGTAT | |
| | TAGGTGCAATATTAGGTGGTGGAAAAGAAGAAGAGGTAGCAAA | |
| | ACTTCGTAAATTCGCTAACTGCATTGGTTTACTTTTCCAAGTAGT | |
| | AGATGATATTCTTGATGTAACAAAATCATCTAAAGAATTAGGTA | |
| | AAACAGCAGGTAAAGATTTAGTTGCTGATAAAACTACTTATCCA | |
| | AAATTAATCGGTGTTGAGAAAAGTAAAGAGTTCGCAGACCGTTT | |
| | AAATCGTGAAGCTCAAGAACAACTTCTTCATTTTCATCCACATA | |
| | GAGCAGCACCTTTAATCGCTTTAGCAAACTATATTGCTTATCGTG | |
| | ATAATGGTACCGGTGAAAATTTATATTTTCAAGGTTCAGGTGGC | |
| | GGAGGTTCTGATTATAAAGATGATGATGATAAAGGAACCGGTTAA | |
| 181 | ATGGTACCAAGTCAACCTTACTGGGCAGCAATTGAGGCAGATAT | GPPS-SSU (M. spicata) |
| | TGAACGTTACTTAAAAAAATCAATTACAATTCGTCCACCAGAAA | |
| | CTGTATTTGGTCCAATGCACCACTTAACTTTTGCTGCACCAGCTA | |
| | CAGCTGCTAGTACTTTATGTTTAGCAGCATGTGAACTTGTAGGTG | |
| | GTGATCGTAGTCAAGCTATGGCTGCAGCAGCAGCAATCCATCTT | |
| | GTTCATGCAGCTGCTTATGTACATGAACATTTACCATTAACTGAT | |
| | GGTAGTCGTCCAGTAAGTAAACCAGCTATCCAACATAAATATGG | |
| | TCCAAATGTAGAATTACTTACAGGTGACGGTATTGTACCATTTG | |
| | GTTTTGAATTATTAGCAGGTTCTGTTGATCCAGCACGTACAGATG | |
| | ATCCAGACCGTATTTTACGTGTAATAATTGAAATAAGTCGTGCT | |
| | GGTGGTCCAGAAGGTATGATTAGTGGTTTACATCGTGAAGAAGA | |
| | GATTGTAGATGGTAATACTTCTCTTGATTTTATTGAATACGTTTG | |
| | CAAAAAAAAATATGGTGAAATGCACGCATGTGGTGCTGCATGC | |
| | GGTGCAATTTTAGGTGGTGCAGCTGAAGAAGAAATTCAAAAACT | |
| | TCGTAACTTCGGATTATATCAAGGAACTTTACGTGGTATGATGG | |
| | AGATGAAAAACTCACACCAACTTATTGACGAAAATATCATTGGC | |
| | AAACTTAAAGAATTAGCTTTAGAAGAATTAGGTGGATTTCATGG | |
| | TAAAAATGCTGAATTAATGTCTAGTTTAGTAGCAGAACCATCAT | |
| | TATATGCTGCTGGTACCGGTGAAAATTTATACTTTCAAGGTTCTG | |
| | GTGGTGGTGGCAGTGATTATAAAGACGATGATGACAAAGGAAC | |
| | CGGTTAA | |
| 182 | ATGGTACCACTTTTATCTAACAAATTAAGAGAGATGGTTTTAGC | GPPS (A. thaliana) |
| | AGAAGTTCCTAAATTAGCATCTGCTGCTGAATATTTCTTTAAACG | |
| | TGGTGTTCAGGGTAAACAATTCCGTTCAACAATTTTATTATTAAT | |
| | GGCAACAGCTCTTGACGTTCGTGTTCCAGAAGCATTAATTGGTG | |
| | AATCTACTGATATTGTAACATCTGAATTACGTGTACGTCAACGT | |
| | GGCATTGCTGAAATTACAGAAATGATTCATGTAGCATCACTTCT | |
| | TCACGATGACGTTCTTGACGATGCTGATACTCGTCGTGGTGTTGG | |
| | TAGTCTTAATGTTGTAATGGGAAACAAAATGTCAGTTTTAGCAG | |
| | GTGACTTCTTACTTTCTCGTGCTTGTGGTGCTCTTGCAGCTCTTA | |
| | AAAACACAGAAGTTGTAGCATTATTAGCTACAGCAGTAGAACAC | |
| | TTAGTTACTGGTGAGACAATGGAAATAACTTCATCAACTGAACA | |
| | ACGTTATTCTATGGATTACTACATGCAGAAAACTTATTACAAAA | |
| | CTGCTTCATTAATTTCAAATTCATGTAAAGCAGTTGCTGTATTAA | |
| | CAGGTCAAACAGCTGAAGTTGCAGTATTAGCTTTTGAATATGGT | |
| | CGTAATTTAGGTTTAGCTTTCCAGTTAATTGACGACATTTTAGAT | |
| | TTCACAGGCACATCTGCTAGTTTAGGAAAAGGTTCTTTATCAGA | |
| | TATACGTCATGGTGTTATTACTGCTCCTATCTTATTTGCAATGGA | |
| | AGAATTTCCTCAATTAAGAGAAGTAGTAGATCAAGTAGAAAAA | |
| | GATCCAAGAAATGTAGACATAGCTTTAGAATATTTAGGTAAAAG | |
| | TAAAGGTATTCAACGTGCTCGTGAATTAGCAATGGAACACGCAA | |
| | ATTTAGCTGCTGCAGCTATTGGTTCTTTACCTGAAACAGATAACG | |
| | AAGATGTTAAACGTTCACGTCGTGCTTTAATTGATTTAACACAC | |
| | AGAGTAATTACACGTAACAAAGGTACCGGTGAGAATTTATACTT | |
| | TCAAGGTAGTGGTGGAGGAGGTAGTGACTATAAAGATGATGAC | |
| | GATAAAGGAACCGGTTAA | |
| 183 | ATGGTACCAGTAGTTTCTGAACGTTTAAGACATTCTGTAACAAC | GPPS (C. reinhardtii) |
| | TGGTATTCCAGCATTAAAAACAGCAGCTGAATATTTCTTTCGTCG | |
| | TGGTATCGAAGGAAAACGTTTAAGACCTACATTAGCATTATTAA | |
| | TGAGTAGTGCTTTATCACCAGCTGCTCCATCACCAGAGTATTTAC | |
| | AAGTTGATACAAGACCTGCTGCAGAACACCCTCATGAAATGCGT | |
| | CGTCGTCAACAACGTTTAGCTGAAATTGCAGAATTAATCCATGT | |
| | AGCTTCATTACTTCACGATGATGTTATTGATGACGCACAAACAC | |
| | GTCGTGGTGTTTTAAGTTTAAATACATCTGTTGGTAATAAAACA | |
| | GCTATCTTAGCAGGTGATTTCTTATTAGCTCGTGCATCTGTAACA | |
| | TTAGCTAGTTTAAGAAACTCTGAAATTGTAGAATTAATGTCACA | |
| | GGTTTTAGAACACTTAGTATCTGGTGAAATTATGCAAATGACTG | |
| | CTACTTCAGAACAACTTTTAGATTTAGAACATTATTTAGCAAAA | |
| | ACATATTGTAAAACTGCTTCATTAATGGCTAATAGTTCTCGTTCT | |
| | GTTGCAGTTCTTGCAGGTGCAGCTCCTGAAGTTTGTGATATGGC | |
| | ATGGTCATACGGTCGTCATTTAGGTATTGCTTTCCAAGTAGTTGA | |
| | CGATTTATTAGATTTAACAGGTTCATCTTCTGTTTTAGGTAAACC | |
| | TGCTTTAAACGATATGCGTTCTGGTTTAGCAACAGCACCAGTATT | |
| | ATTCGCTGCACAAGAAGAACCTGCATTACAGGCTCTTATATTAC | |
| | GTCGTTTTAAACACGACGGTGACGTAACAAAAGCAATGTCATTA | |
| | ATTGAACGTACACAAGGCTTACGTCGTGCTGAAGAACTTGCAGC | |
| | ACAACACGCAAAAGCTGCTGCTGATATGATTCGTTGCTTACCTA | |
| | CAGCTCAATCAGACCATGCAGAAATTGCTCGTGAAGCATTAATT | |
| | CAAATTACACATCGTGTTTTAACACGTAAAAAAGGTACCGGTGA | |
| | AAACTTATACTTTCAAGGTTCTGGTGGTGGTGGATCAGATTATA | |
| | AAGATGATGATGACAAAGGAACCGGTTAA | |
| 184 | ATGGTACCAACTACAACATTATCATCTAACCTTAACTCACAATTC | GPP Chimera |
| | ATGCAGGTTTACGAGACTCTTAAATCAGAACTTATTCATGACCC | |
| | ATTATTTGAGTTCGATGACGATTCAAGACAATGGGTAGAACGTA | |
| | TGATTGATTATACTGTACCAGGTGGTAAAATGGTTCGTGGTTAT | |
| | AGTGTAGTAGATAGTTATCAATTACTTAAAGGTGAAGAACTTAC | |
| | AGAAGAAGAGGCATTTTTAGCTTGTGCACTTGGTTGGTGTACAG | |
| | AATGGTTTCAAGCATTCATTCTTTTACATGATGATATGATGGATG | |
| | GTAGTCACACAAGACGTGGTCAACCATGTTGGTTTCGTTTACCT | |
| | GAGGTTGGTGCTGTTGCTATTAATGATGGTGTTTTACTTCGTAAT | |
| | CACGTTCACCGTATTCTTAAAAAACATTTTCAAGGTAAAGCATA | |
| | TTATGTTCATTTAGTTGATTTATTCAATGAAACTGAATTTCAAAC | |
| | AATTAGTGGACAAATGATCGACTTAATTACAACATTAGTTGGTG | |
| | AAAAAGACTTATCTAAATATTCATTAAGTATTCATCGTCGTATCG | |
| | TTCAATACAAAACAGCATACTACTCATTTTACTTACCAGTTGCTT | |
| | GTGCTTTACTTATGTTTGGTGAGGATCTTGATAAACATGTAGAA | |
| | GTTAAAAATGTTCTTGTTGAAATGGGTACATATTTTCAAGTTCAA | |
| | GATGATTATTTAGATTGTTTTGGTGCTCCAGAAGTTATTGGCAAA | |
| | ATTGGTACTGATATTGAAGACTTTAAATGTTCATGGTTAGTAGTT | |
| | AAAGCATTAGAATTAGCAAATGAAGAACAGAAAAAAACTTTAC | |
| | ACGAAAATTATGGAAAAAAAGATCCAGCATCAGTTGCTAAAGTT | |
| | AAAGAAGTATACCACACACTTAATTTACAAGCTGTTTTCGAAGA | |
| | TTATGAAGCAACATCATACAAAAAACTTATTACTTCTATTGAAA | |
| | ATCACCCATCTAAAGCTGTTCAAGCTGTTTTAAAATCTTTCTTAG | |
| | GCAAAATATACAAACGTCAAAAAGGTACCGGTGAAAACTTATA | |
| | CTTTCAAGGTTCTGGTGGCGGTGGAAGTGATTACAAAGATGATG | |
| | ACGATAAAGGAACCGGTTAA | |
| 185 | ATGGTACCAAGTCAACCTTACTGGGCTGCAATTGAAGCAGACAT | IS-14-15 fusion |
| | TGAAAGATATTTAAAAAAATCAATTACAATTCGTCCACCAGAAA | |
| | CTGTATTTGGTCCTATGCACCATTTAACATTTGCTGCTCCTGCTA | |
| | CTGCAGCTAGTACATTATGCCTTGCTGCTTGTGAATTAGTTGGCG | |
| | GTGATCGTAGTCAAGCTATGGCAGCTGCTGCTGCTATCCATTTA | |
| | GTTCATGCAGCTGCTTACGTTCACGAACATCTTCCTTTAACAGAT | |
| | GGATCACGTCCTGTAAGTAAACCTGCTATTCAACATAAATATGG | |
| | TCCAAACGTTGAACTTTTAACAGGTGATGGTATCGTTCCTTTCGG | |
| | TTTTGAGTTATTAGCAGGTTCAGTAGATCCAGCACGTACTGATG | |
| | ACCCTGATCGTATTTTACGTGTAATTATTGAAATTTCTCGTGCTG | |
| | GTGGACCAGAAGGCATGATTTCTGGTTTACACCGTGAGGAAGAA | |
| | ATCGTAGATGGTAACACATCATTAGACTTTATAGAATATGTATG | |
| | CAAAAAAAAATACGGTGAAATGCACGCATGTGGTGCAGCTTGC | |
| | GGAGCTATTTTAGGTGGAGCTGCTGAAGAAGAAATTCAAAAACT | |
| | TCGTAACTTTGGTCTTTATCAAGGCACATTACGTGGTATGATGGA | |
| | AATGAAAAATAGTCATCAGTTAATTGACGAAAATATCATTGGAA | |
| | AACTTAAAGAACTTGCTCTTGAAGAATTAGGTGGATTCCACGGT | |
| | AAAAACGCTGAATTAATGAGTTCTTTAGTTGCTGAACCTAGTTT | |
| | ATATGCAGCTTCATCAAATAACTTAGGTATCGAAGGTCGTTTTG | |
| | ACTTTGACGGTTACATGCTTCGTAAAGCAAAATCTGTAAATAAA | |
| | GCATTAGAAGCTGCTGTTCAAATGAAAGAACCACTTAAAATTCA | |
| | CGAATCAATGCGTTATTCATTATTAGCTGGTGGTAAACGTGTTCG | |
| | TCCAATGTTATGTATTGCAGCTTGTGAACTTGTTGGTGGTGACGA | |
| | ATCTACAGCAATGCCTGCAGCATGTGCTGTTGAAATGATTCACA | |
| | CAATGTCTTTAATGCATGATGACCTTCCATGTATGGATAACGAT | |
| | GACTTACGTCGTGGTAAACCTACAAACCACATGGCTTTTGGTGA | |
| | GTCTGTAGCTGTTCTTGCTGGTGATGCATTACTTAGTTTTGCTTTT | |
| | GAACATGTTGCTGCTGCAACAAAAGGCGCACCACCTGAACGTAT | |
| | CGTACGTGTATTAGGTGAATTAGCTGTTAGTATTGGTTCAGAAG | |
| | GACTTGTAGCAGGTCAAGTTGTAGACGTTTGTTCTGAAGGCATG | |
| | GCTGAAGTAGGATTAGATCATCTTGAATTTATTCACCATCATAA | |
| | AACTGCTGCATTATTACAAGGTTCAGTTGTTTTAGGTGCAATATT | |
| | AGGAGGCGGTAAAGAAGAAGAAGTAGCTAAACTTCGTAAATTT | |
| | GCTAACTGTATTGGTTTACTTTTCCAAGTTGTTGATGATATTTTA | |
| | GATGTTACTAAAAGTAGTAAAGAGTTAGGTAAAACTGCAGGTA | |
| | AAGACTTAGTAGCTGATAAAACTACATATCCTAAACTTATAGGC | |
| | GTTGAAAAATCAAAAGAATTTGCTGACCGTTTAAATCGTGAAGC | |
| | ACAAGAACAATTATTACATTTTCATCCTCACCGTGCTGCTCCATT | |
| | AATCGCTTTAGCTAACTACATCGCTTACCGTGATAATGGTACCG | |
| | GTGAAAACTTATACTTCCAGGGTAGTGGTGGTGGCGGATCAGAT | |
| | TATAAAGATGACGATGATAAAGGAACCGGTTAA | |
| 186 | ATGGTACCACACAAGTTCACAGGTGTTAACGCTAAATTCCAGCA | IS-09 A118W |
| | ACCAGCATTAAGAAATTTATCTCCAGTGGTAGTTGAGCGCGAAC | (G. gallus) |
| | GTGAGGAATTTGTAGGATTCTTTCCACAAATTGTTCGTGACTTAA | |
| | CTGAAGATGGTATTGGTCATCCAGAAGTAGGTGACGCTGTAGCT | |
| | CGTCTTAAAGAAGTATTACAATACAACGCACCTGGTGGTAAATG | |
| | CAATAGAGGTTTAACAGTTGTTGCAGCTTACCGTGAACTTTCTG | |
| | GACCAGGTCAAAAAGACGCTGAAAGTCTTCGTTGTGCTTTAGCA | |
| | GTAGGATGGTGTATTGAATTATTCCAAGCCTTTTTCTTAGTTTGG | |
| | GACGATATAATGGACCAGTCATTAACTAGACGTGGTCAATTATG | |
| | TTGGTACAAGAAAGAAGGTGTTGGTTTAGATGCAATAAATGATT | |
| | CTTTTCTTTTAGAAAGCTCTGTGTATCGCGTTCTTAAAAAGTATT | |
| | GCCGTCAACGTCCATATTATGTACATTTATTAGAGCTTTTTCTTC | |
| | AAACAGCTTACCAAACAGAATTAGGACAAATGTTAGATTTAATC | |
| | ACTGCTCCTGTATCTAAGGTAGATTTAAGCCATTTCTCAGAAGA | |
| | ACGTTACAAAGCTATTGTTAAGTATAAAACTGCTTTCTATTCATT | |
| | CTATTTACCAGTTGCAGCAGCTATGTATATGGTTGGTATAGATTC | |
| | TAAAGAAGAACATGAAAACGCAAAAGCTATTTTACTTGAGATG | |
| | GGTGAATACTTCCAAATTCAAGATGATTATTTAGATTGTTTTGGC | |
| | GATCCTGCTTTAACAGGTAAAGTAGGTACTGATATTCAAGATAA | |
| | CAAATGTTCATGGTTAGTTGTGCAATGCTTACAAAGAGTAACAC | |
| | CAGAACAACGTCAACTTTTAGAAGATAATTACGGTCGTAAAGAA | |
| | CCAGAAAAAGTTGCTAAAGTTAAAGAATTATATGAGGCTGTAGG | |
| | TATGAGAGCCGCCTTTCAACAATACGAAGAAAGTAGTTACCGTC | |
| | GTCTTCAAGAGTTAATTGAGAAACATTCTAATCGTTTACCAAAA | |
| | GAAATTTTCTTAGGTTTAGCTCAGAAAATATACAAACGTCAAAA | |
| | AGGTACCGGTGAAAACTTATACTTTCAAGGCTCAGGTGGCGGTG | |
| | GAAGTGATTACAAAGATGATGATGATAAAGGAACCGGTTAA | |
| 187 | ATGGTACCACACAAGTTCACAGGTGTTAACGCTAAATTCCAGCA | FPP (G. gallus) |
| | ACCAGCATTAAGAAATTTATCTCCAGTGGTAGTTGAGCGCGAAC | |
| | GTGAGGAATTTGTAGGATTCTTTCCACAAATTGTTCGTGACTTAA | |
| | CTGAAGATGGTATTGGTCATCCAGAAGTAGGTGACGCTGTAGCT | |
| | CGTCTTAAAGAAGTATTACAATACAACGCACCTGGTGGTAAATG | |
| | CAATAGAGGTTTAACAGTTGTTGCAGCTTACCGTGAACTTTCTG | |
| | GACCAGGTCAAAAAGACGCTGAAAGTCTTCGTTGTGCTTTAGCA | |
| | GTAGGATGGTGTATTGAATTATTCCAAGCCTTTTTCTTAGTTGCT | |
| | GACGATATAATGGACCAGTCATTAACTAGACGTGGTCAATTATG | |
| | TTGGTACAAGAAAGAAGGTGTTGGTTTAGATGCAATAAATGATT | |
| | CTTTTCTTTTAGAAAGCTCTGTGTATCGCGTTCTTAAAAAGTATT | |
| | GCCGTCAACGTCCATATTATGTACATTTATTAGAGCTTTTTCTTC | |
| | AAACAGCTTACCAAACAGAATTAGGACAAATGTTAGATTTAATC | |
| | ACTGCTCCTGTATCTAAGGTAGATTTAAGCCATTTCTCAGAAGA | |
| | ACGTTACAAAGCTATTGTTAAGTATAAAACTGCTTTCTATTCATT | |
| | CTATTTACCAGTTGCAGCAGCTATGTATATGGTTGGTATAGATTC | |
| | TAAAGAAGAACATGAAAACGCAAAAGCTATTTTACTTGAGATG | |
| | GGTGAATACTTCCAAATTCAAGATGATTATTTAGATTGTTTTGGC | |
| | GATCCTGCTTTAACAGGTAAAGTAGGTACTGATATTCAAGATAA | |
| | CAAATGTTCATGGTTAGTTGTGCAATGCTTACAAAGAGTAACAC | |
| | CAGAACAACGTCAACTTTTAGAAGATAATTACGGTCGTAAAGAA | |
| | CCAGAAAAAGTTGCTAAAGTTAAAGAATTATATGAGGCTGTAGG | |
| | TATGAGAGCCGCCTTTCAACAATACGAAGAAAGTAGTTACCGTC | |
| | GTCTTCAAGAGTTAATTGAGAAACATTCTAATCGTTTACCAAAA | |
| | GAAATTTTCTTAGGTTTAGCTCAGAAAATATACAAACGTCAAAA | |
| | AGGTACCGGTGAAAACTTATACTTTCAAGGCTCAGGTGGCGGTG | |
| | GAAGTGATTACAAAGATGATGATGATAAAGGAACCGGTTAA | |
| 188 | ATGGTACCAGATTTTCCACAACAATTAGAAGCATGTGTTAAACA | FPP (E. coli) |
| | AGCAAATCAAGCATTATCACGTTTCATCGCACCACTTCCATTCCA | |
| | AAATACTCCTGTTGTTGAAACAATGCAATATGGTGCATTATTAG | |
| | GAGGTAAAAGATTAAGACCATTTCTTGTATATGCAACAGGTCAC | |
| | ATGTTTGGAGTATCTACTAACACATTAGATGCTCCAGCTGCTGC | |
| | AGTTGAATGTATTCATGCATATAGTTTAATTCATGATGATTTACC | |
| | TGCAATGGATGATGATGACTTAAGAAGAGGTTTACCTACATGTC | |
| | ATGTTAAATTTGGTGAAGCTAATGCTATTTTAGCTGGCGATGCA | |
| | CTTCAAACTCTTGCATTCAGTATTTTATCAGATGCTGATATGCCA | |
| | GAAGTTTCAGATCGTGATCGTATTTCTATGATATCTGAATTAGCT | |
| | TCTGCTAGTGGTATTGCTGGTATGTGCGGTGGCCAAGCTCTTGAT | |
| | TTAGACGCAGAAGGAAAACACGTTCCTTTAGATGCTTTAGAGCG | |
| | TATACATCGTCACAAAACAGGAGCTTTAATTAGAGCTGCTGTTC | |
| | GTCTTGGTGCTTTATCAGCTGGAGACAAAGGTCGTCGTGCTTTAC | |
| | CAGTTTTAGACAAATACGCTGAAAGTATTGGTTTAGCTTTTCAA | |
| | GTTCAGGATGATATCTTAGATGTTGTAGGTGATACTGCTACTTTA | |
| | GGTAAACGTCAAGGTGCTGATCAACAGTTAGGCAAATCTACATA | |
| | CCCAGCACTTTTAGGTTTAGAACAAGCTCGTAAAAAAGCAAGAG | |
| | ACTTAATTGACGATGCTCGTCAAAGTCTTAAACAATTAGCAGAA | |
| | CAATCACTTGATACAAGTGCTTTAGAAGCATTAGCAGATTACAT | |
| | TATTCAACGTAATAAAGGTACCGGTGAAAATTTATATTTTCAAG | |
| | GTTCTGGTGGTGGAGGTTCAGACTATAAAGATGACGATGATAAA | |
| | GGAACCGGTTAA | |
| 189 | ATGGTACCAAGTGTTAGTTGTTGTTGTAGAAATTTAGGAAAAAC | FPP (A. thaliana) |
| | TATCAAAAAAGCTATTCCAAGTCACCACTTACATTTACGTTCTTT | |
| | AGGTGGTAGTTTATATAGAAGACGTATTCAATCATCTTCAATGG | |
| | AAACAGACTTAAAATCTACATTCTTAAATGTTTATTCAGTTCTTA | |
| | AATCAGATTTATTACACGACCCATCATTTGAATTTACAAATGAA | |
| | AGTCGTTTATGGGTAGATAGAATGCTTGATTATAATGTTCGTGG | |
| | CGGTAAACTTAATCGTGGTCTTTCTGTAGTAGACTCTTTCAAATT | |
| | ACTTAAACAAGGTAATGATTTAACTGAACAAGAAGTTTTCTTAT | |
| | CTTGTGCATTAGGTTGGTGTATTGAGTGGTTACAGGCTTACTTTT | |
| | TAGTTCTTGATGATATTATGGATAATTCAGTTACACGTCGTGGTC | |
| | AACCTTGTTGGTTTCGTGTACCACAAGTTGGTATGGTAGCTATTA | |
| | ATGATGGCATTCTTCTTCGTAACCATATTCATCGTATTCTTAAAA | |
| | AACACTTCCGTGATAAACCATATTATGTAGATTTAGTTGACCTTT | |
| | TCAATGAAGTAGAGTTACAAACTGCATGTGGACAAATGATTGAT | |
| | TTAATCACAACATTTGAAGGTGAAAAAGACTTAGCTAAATATAG | |
| | TTTATCAATTCACCGTCGTATTGTTCAATACAAAACTGCATATTA | |
| | CTCATTCTATTTACCAGTTGCATGTGCTCTTTTAATGGCTGGCGA | |
| | AAATTTAGAAAACCACATTGATGTTAAAAATGTATTAGTAGATA | |
| | TGGGTATTTACTTTCAAGTTCAGGATGATTATTTAGACTGTTTTG | |
| | CTGATCCTGAAACATTAGGTAAAATTGGCACTGATATTGAGGAC | |
| | TTTAAATGTTCTTGGTTAGTTGTAAAAGCATTAGAACGTTGTAGT | |
| | GAAGAACAAACAAAAATTCTTTACGAAAACTATGGCAAACCTG | |
| | ATCCATCTAATGTTGCTAAAGTAAAAGATTTATACAAAGAATTA | |
| | GATTTAGAAGGCGTTTTCATGGAATATGAATCTAAATCATACGA | |
| | GAAATTAACTGGTGCTATCGAAGGTCACCAATCTAAAGCAATTC | |
| | AAGCTGTTCTTAAATCTTTCTTAGCAAAAATCTATAAACGTCAA | |
| | AAAGGTACCGGTGAAAACTTATACTTTCAAGGTAGTGGTGGCGG | |
| | TGGTAGTGATTATAAAGATGATGATGATAAAGGAACCGGTTAA | |
| 190 | ATGGTACCAGCTGATCTTAAATCAACATTCTTAGATGTTTATTCA | FPP (A. thaliana) |
| | GTATTAAAAAGTGATTTATTACAAGATCCATCTTTTGAATTTACA | |
| | CACGAAAGTCGTCAATGGTTAGAACGTATGTTAGATTATAATGT | |
| | TCGTGGAGGCAAATTAAACAGAGGTTTAAGTGTAGTAGACAGTT | |
| | ACAAACTTTTAAAACAAGGTCAAGACTTAACAGAAAAAGAAAC | |
| | ATTTTTATCTTGTGCTTTAGGTTGGTGTATTGAATGGTTACAAGC | |
| | ATACTTCTTAGTTTTAGACGATATTATGGATAATTCTGTAACTAG | |
| | ACGTGGTCAACCATGTTGGTTTCGTAAACCAAAAGTAGGTATGA | |
| | TTGCTATTAATGATGGAATACTTCTTCGTAACCACATTCATCGTA | |
| | TTCTTAAAAAACACTTTCGTGAAATGCCTTATTATGTAGACCTTG | |
| | TAGACTTATTTAACGAAGTAGAATTTCAAACAGCTTGTGGTCAA | |
| | ATGATTGACTTAATTACAACATTTGATGGTGAAAAAGACCTTTC | |
| | AAAATATTCACTTCAGATTCACCGTCGTATTGTTGAGTACAAAA | |
| | CAGCATACTACTCTTTCTATTTACCTGTAGCATGTGCTTTACTTA | |
| | TGGCAGGTGAAAATTTAGAAAATCACACAGATGTTAAAACTGTA | |
| | TTAGTTGATATGGGTATCTATTTCCAAGTTCAAGATGATTATTTA | |
| | GATTGCTTCGCTGATCCAGAAACATTAGGTAAAATTGGTACAGA | |
| | TATTGAAGACTTTAAATGTAGTTGGTTAGTAGTAAAAGCATTAG | |
| | AACGTTGTAGTGAAGAACAAACAAAAATTCTTTACGAAAATTAT | |
| | GGAAAAGCTGAACCTTCAAATGTAGCTAAAGTTAAAGCATTATA | |
| | CAAAGAATTAGATTTAGAGGGTGCATTTATGGAATATGAAAAAG | |
| | AATCATACGAGAAACTTACAAAACTTATTGAAGCACATCAATCA | |
| | AAAGCTATTCAAGCAGTTCTTAAATCTTTCTTAGCTAAAATTTAT | |
| | AAACGTCAAAAAGGTACCGGTGAAAACTTATACTTTCAAGGCTC | |
| | TGGAGGTGGTGGTTCAGACTATAAAGATGATGATGATAAAGGA | |
| | ACCGGTTAA | |
| 191 | ATGGTACCAAGTGGCGAACCTACTCCAAAAAAAATGAAAGCAA | FPP (C. reinhardtii) |
| | CATACGTTCACGACCGTGAAAACTTTACAAAAGTATACGAAACT | |
| | CTTCGTGACGAATTACTTAACGATGATTGTCTTAGTCCAGCTGGT | |
| | TCACCTCAGGCTCAAGCTGCTCAAGAGTGGTTTAAAGAAGTTAA | |
| | TGATTATAATGTTCCTGGTGGAAAACTTAACCGTGGTATGGCTG | |
| | TATATGACGTTTTAGCTTCAGTTAAAGGTCCAGATGGTTTAAGTG | |
| | AAGACGAAGTATTTAAAGCTAACGCTCTTGGTTGGTGTATTGAG | |
| | TGGTTACAAGCATTTTTCTTAGTTGCTGATGATATAATGGATGGT | |
| | TCAATTACACGTCGTGGCCAACCTTGTTGGTACAAACAACCTAA | |
| | AGTTGGTATGATTGCTTGTAATGATTACATCTTATTAGAATGCTG | |
| | TATTTACTCAATTCTTAAAAGACATTTTAGAGGTCACGCTGCATA | |
| | CGCTCAACTTATGGACCTTTTCCATGAAACTACATTCCAGACTTC | |
| | ACACGGTCAATTATTAGATTTAACAACAGCACCTATCGGTTCTG | |
| | TAGACTTATCAAAATATACAGAAGATAATTACCTTCGTATTGTA | |
| | ACATATAAAACTGCATACTATTCTTTTTATTTACCTGTAGCATGT | |
| | GGTATGGTATTAGCTGGCATTACAGATCCAGCTGCTTTTGATCTT | |
| | GCAAAAAATATTTGTGTTGAAATGGGTCAATATTTCCAGATTCA | |
| | AGACGATTATTTAGATTGCTATGGTGACCCTGAGGTTATTGGTA | |
| | AAATCGGTACAGACATAGAAGACAACAAATGTAGTTGGTTAGTT | |
| | TGCACAGCTCTTAAAATCGCAACAGAAGAACAAAAAGAGGTTA | |
| | TAAAAGCTAATTATGGTCACAAAGAGGCTGAATCAGTAGCAGC | |
| | AATTAAAGCATTATACGTTGAATTAGGTATTGAACAACGTTTTA | |
| | AAGACTATGAAGCTGCATCATACGCAAAATTAGAAGGTACAATT | |
| | AGTGAACAAACTTTATTACCTAAAGCAGTATTTACTTCTTTATTA | |
| | GCTAAAATCTATAAAAGAAAAAAAGGTACCGGTGAGAACTTAT | |
| | ACTTTCAAGGTAGTGGAGGTGGTGGTTCAGACTATAAAGATGAT | |
| | GATGATAAAGGAACCGGTTAA | |
| 192 | ATGGTACCAGTAACAGCAGCACGTGCAACACCAAAATTAAGTA | Geranylgeranyl |
| | ATAGAAAATTACGTGTTGCTGTAATTGGAGGCGGTCCAGCAGGA | reductase (A. thaliana) |
| | GGTGCAGCTGCTGAAACATTAGCACAAGGAGGTATTGAAACAA | |
| | TTCTTATCGAACGTAAAATGGATAATTGTAAACCATGTGGTGGT | |
| | GCTATTCCATTATGTATGGTAGGAGAGTTCAATTTACCTTTAGAC | |
| | ATTATTGACCGTCGTGTAACAAAAATGAAAATGATCTCTCCTTC | |
| | AAACATTGCAGTTGATATCGGTCGTACACTTAAAGAACACGAAT | |
| | ATATTGGTATGGTTCGTCGTGAGGTACTTGATGCTTATCTTCGTG | |
| | AACGTGCAGAAAAATCAGGTGCTACTGTTATTAACGGTTTATTC | |
| | TTAAAAATGGATCACCCAGAAAATTGGGATTCACCATATACACT | |
| | TCACTACACAGAGTATGATGGAAAAACAGGTGCTACAGGAACT | |
| | AAAAAAACTATGGAAGTAGATGCTGTTATTGGTGCTGATGGTGC | |
| | TAATTCTCGTGTTGCAAAAAGTATTGACGCAGGTGATTATGATT | |
| | ATGCTATTGCATTTCAAGAACGTATTCGTATACCTGATGAGAAA | |
| | ATGACTTATTATGAGGACTTAGCTGAGATGTATGTAGGTGATGA | |
| | TGTATCACCAGACTTCTACGGTTGGGTATTCCCAAAATGTGATC | |
| | ATGTAGCTGTTGGTACAGGTACTGTAACACATAAAGGTGATATC | |
| | AAAAAATTCCAGTTAGCTACACGTAATCGTGCTAAAGATAAAAT | |
| | TCTTGGTGGCAAAATAATCCGTGTAGAGGCTCATCCTATTCCAG | |
| | AGCATCCTAGACCACGTCGTTTATCAAAACGTGTTGCATTAGTA | |
| | GGCGACGCAGCAGGTTACGTTACTAAATGTTCAGGAGAAGGAA | |
| | TTTACTTCGCAGCTAAATCTGGTCGTATGTGTGCTGAAGCTATCG | |
| | TTGAAGGTTCACAAAATGGCAAAAAAATGATAGATGAAGGCGA | |
| | TTTAAGAAAATACTTAGAAAAATGGGATAAAACTTACTTACCAA | |
| | CTTATCGTGTTTTAGATGTACTTCAAAAAGTTTTCTATCGTTCTA | |
| | ACCCAGCTCGTGAGGCTTTTGTTGAAATGTGTAACGATGAGTAT | |
| | GTACAGAAAATGACATTTGATTCTTACCTTTATAAACGTGTAGCT | |
| | CCTGGTAGTCCATTAGAAGATATCAAATTAGCTGTAAATACTAT | |
| | TGGTTCACTTGTTCGTGCTAACGCATTACGTCGTGAAATTGAGA | |
| | AATTATCAGTAGGTACCGGTGAGAATCTTTACTTTCAAGGATCA | |
| | GGTGGTGGTGGTTCTGATTATAAAGATGACGATGATAAAGGAAC | |
| | CGGTTAA | |
| 193 | ATGGTACCAGTAGCTGTTATTGGTGGTGGTCCAAGTGGCGCTTG | Geranyl-geranyl |
| | TGCAGCAGAAACTTTAGCAAAAGGTGGTGTAGAAACTTTCTTAC | reductase (C. reinhardtii) |
| | TTGAGCGTAAATTAGATAATTGTAAACCTTGTGGAGGTGCAATT | |
| | CCATTATGTATGGTTGAAGAATTTGATTTACCAATGGAAATAAT | |
| | TGACCGTCGTGTTACTAAAATGAAAATGATATCACCTTCAAACC | |
| | GTGAAGTTGATGTTGGAAAAACTTTATCAGAAACTGAATGGATC | |
| | GGTATGTGTCGTCGTGAAGTATTTGACGATTACTTAAGAAACCG | |
| | TGCACAGAAATTAGGTGCTAATATTGTTAACGGTTTATTCATGC | |
| | GTTCAGAACAACAATCTGCAGAGGGTCCATTCACAATTCACTAT | |
| | AATTCTTATGAAGACGGTAGTAAAATGGGAAAACCTGCTACTTT | |
| | AGAAGTTGATATGATAATTGGTGCAGATGGAGCAAATTCTCGTA | |
| | TTGCAAAAGAGATAGATGCAGGTGAATACGACTACGCTATAGCT | |
| | TTTCAAGAACGTATTCGTATTCCTGATGATAAAATGAAATATTA | |
| | CGAAAACCTTGCTGAAATGTATGTAGGTGATGACGTATCTCCTG | |
| | ATTTCTATGGTTGGGTTTTTCCTAAATATGATCACGTTGCTGTTG | |
| | GTACAGGTACTGTTGTAAACAAAACAGCTATTAAACAATATCAA | |
| | CAGGCAACACGTGACAGATCAAAAGTTAAAACAGAAGGTGGCA | |
| | AAATTATACGTGTTGAAGCACACCCAATTCCAGAACATCCACGT | |
| | CCACGTCGTTGTAAAGGTCGTGTTGCATTAGTAGGCGACGCAGC | |
| | TGGTTATGTTACAAAATGTTCTGGCGAGGGCATTTACTTTGCTGC | |
| | TAAATCTGGTAGAATGGCTGCTGAAGCTATTGTAGAAGGTTCTG | |
| | CTAACGGTACAAAAATGTGTGGTGAGGATGCAATTCGTGTTTAT | |
| | TTAGATAAATGGGATCGTAAATATTGGACAACATACAAAGTATT | |
| | AGACATTTTACAAAAAGTATTTTATCGTAGTAATCCAGCACGTG | |
| | AAGCATTTGTTGAATTATGTGAAGATAGTTATGTACAGAAAATG | |
| | ACATTTGATTCATACTTATATAAAACTGTTGTTCCAGGAAACCCA | |
| | TTAGACGACGTAAAATTACTTGTTCGTACAGTATCTTCTATTTTA | |
| | CGTTCAAATGCTTTACGTTCTGTTAATTCTAAATCTGTAAATGTT | |
| | TCTTTCGGCTCTAAAGCAAATGAGGAACGTGTTATGGCTGCAGG | |
| | TACCGGTGAAAATCTTTATTTTCAAGGTTCAGGAGGTGGTGGTT | |
| | CAGATTATAAAGATGATGATGACAAAGGAACCGGTTAA | |
| 194 | ATGGTACCAGCAATGGCAGTACCATTAGATGTAGTAATTACATA | Chlorophyllido- |
| | TCCTTCTTCAGGTGCTGCTGCTTATCCAGTACTTGTTATGTATAA | hydrolase (C. reinhardtii) |
| | CGGTTTCCAAGCTAAAGCTCCATGGTATCGTGGTATTGTAGATC | |
| | ATGTTTCTAGTTGGGGTTACACAGTTGTTCAATATACAAATGGTG | |
| | GCTTATTTCCTATTGTTGTAGATCGTGTTGAGTTAACTTATTTAG | |
| | AGCCATTATTAACTTGGTTAGAAACACAAAGTGCTGATGCTAAA | |
| | TCTCCTTTATACGGTCGTGCAGATGTTTCTCGTTTAGGTACAATG | |
| | GGTCATTCACGTGGTGGTAAATTAGCAGCTTTACAATTTGCTGG | |
| | ACGTACAGATGTAAGTGGTTGTGTATTATTTGACCCTGTAGATG | |
| | GAAGTCCAATGACACCAGAATCTGCTGATTATCCTTCAGCTACA | |
| | AAAGCATTAGCAGCAGCTGGTCGTTCTGCTGGCTTAGTAGGTGC | |
| | AGCTATTACAGGTTCATGTAATCCAGTAGGTCAAAATTACCCAA | |
| | AATTCTGGGGTGCTTTAGCTCCTGGTTCTTGGCAAATGGTATTAT | |
| | CACAAGCTGGTCACATGCAATTTGCTCGTACTGGTAATCCATTCT | |
| | TAGATTGGTCATTAGACCGTTTATGTGGTCGTGGTACAATGATG | |
| | AGTTCAGATGTTATTACATATAGTGCAGCATTTACTGTTGCTTGG | |
| | TTTGAAGGTATTTTTCGTCCTGCTCAAAGTCAAATGGGTATTTCT | |
| | AATTTCAAAACTTGGGCTAATACTCAAGTTGCAGCTCGTAGTAT | |
| | CACTTTTGATATTAAACCTATGCAATCTCCTCAGGGTACCGGTGA | |
| | AAACCTTTACTTTCAAGGTAGTGGTGGTGGAGGAAGTGATTATA | |
| | AAGATGATGATGACAAAGGAACCGGTTAA | |
| 195 | ATGGTACCAGCACCACCAAAACCAGTTCGTATAACTTGTCCAAC | Chlorophyllido- |
| | AGTAGCTGGCACTTATCCTGTTGTTTTATTCTTTCACGGTTTTTAT | hydrolase (A. thaliana) |
| | CTTCGTAACTATTTCTATTCAGATGTTTTAAATCATATTGCTAGT | |
| | CATGGTTACATCTTAGTTGCACCACAATTATGTAAACTTTTACCT | |
| | CCAGGTGGCCAAGTAGAAGTTGATGACGCTGGTTCAGTTATTAA | |
| | CTGGGCTTCAGAGAATCTTAAAGCACACCTTCCAACTTCTGTTA | |
| | ATGCTAATGGTAAATATACATCTTTAGTTGGACATTCACGTGGT | |
| | GGCAAAACAGCTTTCGCAGTTGCATTAGGTCACGCAGCTACATT | |
| | AGATCCATCAATTACATTTTCAGCATTAATTGGTATTGATCCAGT | |
| | AGCAGGAACTAACAAATACATTCGTACAGATCCACACATCTTAA | |
| | CTTATAAACCTGAATCATTTGAATTAGATATTCCTGTAGCTGTTG | |
| | TAGGCACTGGTCTTGGTCCAAAATGGAATAACGTAATGCCTCCA | |
| | TGCGCACCTACAGATTTAAACCACGAAGAATTTTACAAAGAATG | |
| | TAAAGCTACTAAAGCTCACTTTGTTGCTGCTGATTATGGTCACAT | |
| | GGACATGTTAGACGACGATCTTCCAGGTTTTGTAGGCTTCATGG | |
| | CTGGTTGTATGTGTAAAAATGGTCAACGTAAAAAATCAGAAATG | |
| | CGTTCTTTTGTAGGTGGTATAGTTGTAGCATTCTTAAAATATTCT | |
| | TTATGGGGTGAAAAAGCTGAAATAAGATTAATTGTTAAAGATCC | |
| | TAGTGTATCTCCTGCTAAATTAGACCCATCACCAGAATTAGAAG | |
| | AAGCATCAGGTATTTTTGTTGGTACCGGTGAAAATCTTTATTTTC | |
| | AAGGTTCAGGTGGAGGTGGTTCTGATTATAAAGATGATGATGAC | |
| | AAAGGAACCGGTTAA | |
| 196 | ATGGTACCAGCTACACCAGTTGAAGAAGGTGATTATCCAGTTGT | Chlorophyllido- |
| | AATGTTATTACATGGCTACCTTTTATATAATTCATTTTATTCACA | hydrolase (A. thaliana) |
| | ATTAATGTTACATGTATCATCTCACGGTTTCATCTTAATTGCTCC | |
| | ACAATTATACTCAATTGCTGGTCCTGATACTATGGATGAAATTA | |
| | AAAGTACTGCTGAGATTATGGACTGGTTATCAGTTGGTTTAAAT | |
| | CACTTTTTACCAGCTCAAGTTACACCTAATTTATCTAAATTTGCA | |
| | TTATCTGGTCATAGTCGTGGTGGTAAAACTGCTTTTGCTGTAGCA | |
| | TTAAAAAAATTTGGTTATTCTTCAAACTTAAAAATTAGTACTTTA | |
| | ATTGGTATTGATCCAGTAGACGGAACAGGTAAAGGTAAACAAA | |
| | CTCCACCTCCTGTTTTAGCATATTTACCTAATAGTTTTGACTTAG | |
| | ACAAAACACCAATTTTAGTAATTGGTTCAGGTTTAGGTGAAACT | |
| | GCACGTAATCCTTTATTTCCTCCATGTGCTCCTCCAGGTGTTAAC | |
| | CACCGTGAGTTTTTCCGTGAATGTCAAGGTCCAGCATGGCACTTT | |
| | GTTGCTAAAGATTATGGTCATTTAGACATGCTTGATGATGATAC | |
| | AAAAGGTATTCGTGGCAAATCTAGTTACTGTTTATGCAAAAATG | |
| | GTGAAGAACGTCGTCCAATGCGTCGTTTCGTTGGTGGTTTAGTTG | |
| | TTAGTTTTCTTAAAGCATATCTTGAAGGTGATGATCGTGAATTAG | |
| | TAAAAATCAAAGATGGTTGTCATGAAGATGTACCTGTTGAAATT | |
| | CAAGAATTTGAAGTAATTATGGGTACCGGTGAAAATCTTTACTT | |
| | TCAAGGTTCAGGCGGTGGAGGTTCAGATTATAAAGATGATGATG | |
| | ACAAAGGAACCGGTTAA | |
| 197 | ATGGTACCAGCTGCTGCTGCACCTGCTGAGACAATGAATAAATC | Chloro-phyllido- |
| | TGCAGCTGGCGCTGAAGTACCAGAGGCTTTCACATCAGTTTTTC | hydrolase (T. Aestivum) |
| | AACCAGGTAAATTAGCAGTTGAAGCAATTCAAGTAGATGAAAA | |
| | TGCAGCTCCTACTCCACCTATTCCTGTTTTAATAGTTGCTCCAAA | |
| | AGATGCTGGTACATATCCAGTTGCTATGTTATTACACGGATTTTT | |
| | CTTACATAATCACTTTTATGAACACTTATTACGTCACGTTGCATC | |
| | TCATGGCTTTATCATTGTTGCTCCACAATTTTCTATTAGTATTATT | |
| | CCATCAGGAGATGCTGAAGACATCGCTGCTGCTGCAAAAGTAGC | |
| | AGATTGGTTACCTGACGGATTACCAAGTGTTTTACCAAAAGGTG | |
| | TTGAACCAGAGTTATCAAAACTTGCTTTAGCTGGACACAGTCGT | |
| | GGTGGTCACACAGCTTTTTCTTTAGCTTTAGGTCACGCTAAAACA | |
| | CAATTAACTTTCAGTGCATTAATTGGTTTAGATCCTGTTGCTGGA | |
| | ACAGGTAAATCATCTCAATTACAACCAAAAATTCTTACTTATGA | |
| | GCCAAGTTCATTTGGTATGGCTATGCCAGTTTTAGTTATTGGTAC | |
| | AGGTTTAGGAGAAGAAAAAAAAAACATTTTCTTTCCTCCATGTG | |
| | CTCCTAAAGACGTAAACCATGCAGAATTTTATCGTGAATGTAGA | |
| | CCACCATGTTACTATTTTGTAACTAAAGATTATGGCCATCTTGAT | |
| | ATGTTAGATGATGACGCTCCAAAATTTATCACATGTGTTTGTAA | |
| | AGACGGTAATGGATGTAAAGGAAAAATGCGTCGTTGTGTAGCTG | |
| | GCATCATGGTTGCTTTCTTAAACGCTGCTTTAGGTGAAAAAGAC | |
| | GCAGATTTAGAAGCTATTTTACGTGATCCAGCAGTTGCTCCTAC | |
| | AACATTAGACCCAGTTGAACACCGTGTTGCTGGTACCGGTGAGA | |
| | ATTTATACTTCCAGGGATCTGGTGGTGGTGGCAGTGATTATAAA | |
| | GATGATGATGATAAAGGAACCGGTTAA | |
| 198 | ATGGTACCAAGTCACAAAAAAAAAAACGTAATCTTCTTCGTAAC | Phosphatase (S. cerevisiae) |
| | TGATGGTATGGGTCCTGCTTCTCTTTCAATGGCTCGTTCATTTAA | |
| | TCAACACGTTAATGATTTACCAATTGATGATATTTTAACATTAGA | |
| | TGAACATTTTATTGGAAGTTCAAGAACACGTTCATCAGATTCAC | |
| | TTGTAACTGACTCAGCTGCTGGAGCTACAGCTTTTGCTTGTGCAC | |
| | TTAAATCATACAATGGTGCTATAGGTGTAGATCCACACCATCGT | |
| | CCATGTGGAACTGTTTTAGAAGCTGCTAAATTAGCAGGTTATTT | |
| | AACAGGATTAGTAGTTACTACACGTATTACTGATGCTACACCAG | |
| | CTAGTTTCTCAAGTCACGTAGATTATCGTTGGCAAGAAGATTTA | |
| | ATTGCAACACACCAATTAGGTGAATATCCTTTAGGACGTGTTGT | |
| | TGATCTTCTTATGGGTGGTGGTCGTTCTCACTTTTATCCTCAAGG | |
| | TGAAAAAGCTAGTCCATACGGTCACCACGGTGCACGTAAAGATG | |
| | GTCGTGATTTAATCGATGAAGCTCAAAGTAATGGCTGGCAGTAT | |
| | GTAGGAGATCGTAAAAATTTTGATTCTTTACTTAAATCACATGGT | |
| | GAAAATGTTACTTTACCATTTTTAGGTTTATTTGCTGACAACGAT | |
| | ATCCCATTTGAAATTGATCGTGATGAAAAAGAATATCCTAGTTT | |
| | AAAAGAACAAGTAAAAGTAGCATTAGGTGCTTTAGAAAAAGCA | |
| | AGTAACGAAGATAAAGATAGTAATGGTTTCTTTTTAATGGTAGA | |
| | AGGTTCTCGTATTGATCATGCTGGCCATCAAAACGATCCTGCAT | |
| | CTCAAGTACGTGAAGTATTAGCATTTGATGAGGCTTTTCAATAT | |
| | GTATTAGAATTTGCAGAAAACAGTGATACAGAAACAGTATTAGT | |
| | AAGTACATCAGATCATGAAACAGGTGGTTTAGTTACTTCAAGAC | |
| | AAGTAACAGCATCATACCCACAATATGTATGGTATCCTCAAGTA | |
| | TTAGCTAACGCTACACATAGTGGAGAGTTTCTTAAACGTAAATT | |
| | AGTTGATTTCGTTCATGAACACAAAGGCGCATCATCAAAAATAG | |
| | AAAACTTCATAAAACACGAAATTCTTGAAAAAGATTTAGGTATT | |
| | TATGATTATACAGATTCTGACTTAGAAACACTTATTCATTTAGAT | |
| | GATAACGCTAATGCAATTCAAGATAAACTTAATGATATGGTAAG | |
| | TTTTAGAGCTCAAATTGGTTGGACAACACATGGTCATTCAGCAG | |
| | TTGATGTAAACATATATGCTTACGCAAACAAAAAAGCTACATGG | |
| | TCTTATGTTCTTAATAACTTACAAGGTAATCACGAAAACACAGA | |
| | AGTTGGTCAATTCTTAGAGAATTTCTTAGAATTAAACTTAAATG | |
| | AAGTTACTGATTTAATCCGTGATACAAAACATACTTCTGATTTTG | |
| | ACGCAACAGAAATAGCAAGTGAGGTTCAACACTATGATGAATA | |
| | TTACCACGAATTAACAAATGGTACCGGTGAAAATCTTTATTTTC | |
| | AAGGTTCTGGTGGAGGTGGCAGTGATTATAAAGATGATGATGAC | |
| | AAAGGAACCGGTTAA | |
| 199 | ATGGTACCAGCTTTATACGACATTATTAACTATTTCTACGGTTCA | Phosphatase (C. albicans) |
| | AACTCTAAATTCAACCGTATTACATGGGGTTTTAAATCACCAAC | |
| | TTTCATCAAATGGAGAATTACTGATTTCATTTTAATCATCGTTTT | |
| | AATTGTTCTTTTCTTCGTAACTTCTCAAGCAGAGCCATTCCATCG | |
| | TCAATTTTATCTTAACGACATGACTATCCAACATCCTTTTGCAGA | |
| | ACATGAACGTGTAACTAATATTCAACTTGGTTTATATTCAACAGT | |
| | AATTCCTTTATCAGTTATTATCATTGTTGCTTTAATTAGTACATGT | |
| | CCACCTAAATACAAATTATACAACACTTGGGTTTCAAGTATTGG | |
| | TTTACTTTTATCAGTTTTAATCACATCTTTTGTTACAAACATCGTT | |
| | AAAAACTGGTTTGGACGTTTACGTCCTGACTTCTTAGATCGTTGC | |
| | CAACCAGCTAACGATACACCTAAAGATAAATTAGTTTCTATTGA | |
| | GGTTTGTACTACAGACAATTTAGACCGTTTAGCTGACGGTTTTCG | |
| | TACAACACCTTCTGGTCATTCTTCAATCTCATTTGCTGGTTTATTC | |
| | TATTTAACATTATTTCTTTTAGGTCAATCTCAGGCAAATAATGGT | |
| | AAAACATCTTCATGGCGTACAATGATCAGTTTTATACCTTGGTTA | |
| | ATGGCTTGTTATATCGCTTTAAGTCGTACACAAGACTACCGTCAT | |
| | CATTTCATTGACGTATTTGTTGGTAGTTGCTTAGGCTTAATTATC | |
| | GCAATTTGGCAATACTTCCGTTTATTCCCTTGGTTCGGTGGTAAC | |
| | CAAGCAAATGATTCATTTAACAACCGTATTATGATTGAAGAGAT | |
| | TAAACGTAAAGAGGAAATTAAACAAGATGAAAATAACTACCGT | |
| | CGTATTTCTGATATTTCTACTAATGTAGGTACCGGTGAAAACCTT | |
| | TACTTTCAAGGTTCAGGTGGCGGCGGTTCAGATTATAAAGATGA | |
| | TGACGACAAAGGAACCGGTTAA | |

TABLE 8

Nucleic acids encoding exemplary isoprenoid producing
enzymes used to increase phytol production
(with restriction enzyme sites)

| | | Enzyme |
|---|---|---|
| SEQ | | (synthase) |
| ID NO. | Codon-biased, Synthesized Gene Sequence w/Restriction Sites | encoded |
| 200 | CATATGGTACCAGCTTTATACGACATTATTAACTATTTCTACGGT | GPPS-LSU (M. spicata) |
| | TCAAACTCTAAATTCAACCGTATTACATGGGGTTTTAAATCACC | |
| | AACTTTCATCAAATGGAGAATTACTGATTTCATTTTAATCATCGT | |
| | TTTAATTGTTCTTTTCTTCGTAACTTCTCAAGCAGAGCCATTCCA | |
| | TCGTCAATTTTATCTTAACGACATGACTATCCAACATCCTTTTGC | |
| | AGAACATGAACGTGTAACTAATATTCAACTTGGTTTATATTCAA | |
| | CAGTAATTCCTTTATCAGTTATTATCATTGTTGCTTTAATTAGTA | |
| | CATGTCCACCTAAATACAAATTATACAACACTTGGGTTTCAAGT | |
| | ATTGGTTTACTTTTATCAGTTTTAATCACATCTTTTGTTACAAAC | |
| | ATCGTTAAAAACTGGTTTGGACGTTTACGTCCTGACTTCTTAGAT | |
| | CGTTGCCAACCAGCTAACGATACACCTAAAGATAAATTAGTTTC | |
| | TATTGAGGTTTGTACTACAGACAATTTAGACCGTTTAGCTGACG | |
| | GTTTTCGTACAACACCTTCTGGTCATTCTTCAATCTCATTTGCTG | |
| | GTTTATTCTATTTAACATTATTTCTTTTAGGTCAATCTCAGGCAA | |
| | ATAATGGTAAAACATCTTCATGGCGTACAATGATCAGTTTTATA | |
| | CCTTGGTTAATGGCTTGTTATATCGCTTTAAGTCGTACACAAGAC | |
| | TACCGTCATCATTTCATTGACGTATTTGTTGGTAGTTGCTTAGGC | |
| | TTAATTATCGCAATTTGGCAATACTTCCGTTTATTCCCTTGGTTC | |
| | GGTGGTAACCAAGCAAATGATTCATTTAACAACCGTATTATGAT | |
| | TGAAGAGATTAAACGTAAAGAGGAAATTAAACAAGATGAAAAT | |
| | AACTACCGTCGTATTTCTGATATTTCTACTAATGTAGGTACCGGT | |
| | GAAAACCTTTACTTTCAAGGTTCAGGTGGCGGCGGTTCAGATTA | |
| | TAAAGATGATGACGACAAAGGAACCGGTTAATCTAGACTCGAG | |
| 201 | CATATGGTACCAAGTCAACCTTACTGGGCAGCAATTGAGGCAGA | GPPS-SSU (M. spicata) |
| | TATTGAACGTTACTTAAAAAAATCAATTACAATTCGTCCACCAG | |
| | AAACTGTATTTGGTCCAATGCACCACTTAACTTTTGCTGCACCAG | |
| | CTACAGCTGCTAGTACTTTATGTTTAGCAGCATGTGAACTTGTAG | |
| | GTGGTGATCGTAGTCAAGCTATGGCTGCAGCAGCAGCAATCCAT | |
| | CTTGTTCATGCAGCTGCTTATGTACATGAACATTTACCATTAACT | |
| | GATGGTAGTCGTCCAGTAAGTAAACCAGCTATCCAACATAAATA | |
| | TGGTCCAAATGTAGAATTACTTACAGGTGACGGTATTGTACCAT | |
| | TTGGTTTTGAATTATTAGCAGGTTCTGTTGATCCAGCACGTACAG | |
| | ATGATCCAGACCGTATTTTACGTGTAATAATTGAAATAAGTCGT | |
| | GCTGGTGGTCCAGAAGGTATGATTAGTGGTTTACATCGTGAAGA | |
| | AGAGATTGTAGATGGTAATACTTCTCTTGATTTTATTGAATACGT | |
| | TTGCAAAAAAAAATATGGTGAAATGCACGCATGTGGTGCTGCAT | |
| | GCGGTGCAATTTTAGGTGGTGCAGCTGAAGAAGAAATTCAAAA | |
| | ACTTCGTAACTTCGGATTATATCAAGGAACTTTACGTGGTATGAT | |
| | GGAGATGAAAAACTCACACCAACTTATTGACGAAAATATCATTG | |
| | GCAAACTTAAAGAATTAGCTTTAGAAGAATTAGGTGGATTTCAT | |
| | GGTAAAAATGCTGAATTAATGTCTAGTTTAGTAGCAGAACCATC | |
| | ATTATATGCTGCTGGTACCGGTGAAAATTTATACTTTCAAGGTTC | |
| | TGGTGGTGGTGGCAGTGATTATAAAGACGATGATGACAAAGGA | |
| | ACCGGTTAATCTAGACTCGAG | |
| 202 | CATATGGTACCACTTTTATCTAACAAATTAAGAGAGATGGTTTT | GPPS (A. thaliana) |
| | AGCAGAAGTTCCTAAATTAGCATCTGCTGCTGAATATTTCTTTAA | |
| | ACGTGGTGTTCAGGGTAAACAATTCCGTTCAACAATTTTATTATT | |
| | AATGGCAACAGCTCTTGACGTTCGTGTTCCAGAAGCATTAATTG | |
| | GTGAATCTACTGATATTGTAACATCTGAATTACGTGTACGTCAA | |
| | CGTGGCATTGCTGAAATTACAGAAATGATTCATGTAGCATCACT | |
| | TCTTCACGATGACGTTCTTGACGATGCTGATACTCGTCGTGGTGT | |
| | TGGTAGTCTTAATGTTGTAATGGGAAACAAAATGTCAGTTTTAG | |
| | CAGGTGACTTCTTACTTTCTCGTGCTTGTGGTGCTCTTGCAGCTC | |
| | TTAAAAACACAGAAGTTGTAGCATTATTAGCTACAGCAGTAGAA | |
| | CACTTAGTTACTGGTGAGACAATGGAAATAACTTCATCAACTGA | |
| | ACAACGTTATTCTATGGATTACTACATGCAGAAAACTTATTACA | |
| | AAACTGCTTCATTAATTTCAAATTCATGTAAAGCAGTTGCTGTAT | |
| | TAACAGGTCAAACAGCTGAAGTTGCAGTATTAGCTTTTGAATAT | |
| | GGTCGTAATTTAGGTTTAGCTTTCCAGTTAATTGACGACATTTTA | |
| | GATTTCACAGGCACATCTGCTAGTTTAGGAAAAGGTTCTTTATC | |
| | AGATATACGTCATGGTGTTATTACTGCTCCTATCTTATTTGCAAT | |
| | GGAAGAATTTCCTCAATTAAGAGAAGTAGTAGATCAAGTAGAA | |
| | AAAGATCCAAGAAATGTAGACATAGCTTTAGAATATTTAGGTAA | |
| | AAGTAAAGGTATTCAACGTGCTCGTGAATTAGCAATGGAACACG | |
| | CAAATTTAGCTGCTGCAGCTATTGGTTCTTTACCTGAAACAGATA | |
| | ACGAAGATGTTAAACGTTCACGTCGTGCTTTAATTGATTTAACA | |
| | CACAGAGTAATTACACGTAACAAAGGTACCGGTGAGAATTTATA | |
| | CTTTCAAGGTAGTGGTGGAGGAGGTAGTGACTATAAAGATGATG | |
| | ACGATAAAGGAACCGGTTAATCTAGACTCGAG | |
| 203 | CATATGGTACCAGTAGTTTCTGAACGTTTAAGACATTCTGTAAC | GPPS (C. reinhardtii) |
| | AACTGGTATTCCAGCATTAAAAACAGCAGCTGAATATTTCTTTC | |
| | GTCGTGGTATCGAAGGAAAACGTTTAAGACCTACATTAGCATTA | |
| | TTAATGAGTAGTGCTTTATCACCAGCTGCTCCATCACCAGAGTAT | |
| | TTACAAGTTGATACAAGACCTGCTGCAGAACACCCTCATGAAAT | |
| | GCGTCGTCGTCAACAACGTTTAGCTGAAATTGCAGAATTAATCC | |
| | ATGTAGCTTCATTACTTCACGATGATGTTATTGATGACGCACAA | |
| | ACACGTCGTGGTGTTTTAAGTTTAAATACATCTGTTGGTAATAAA | |
| | ACAGCTATCTTAGCAGGTGATTTCTTATTAGCTCGTGCATCTGTA | |
| | ACATTAGCTAGTTTAAGAAACTCTGAAATTGTAGAATTAATGTC | |
| | ACAGGTTTTAGAACACTTAGTATCTGGTGAAATTATGCAAATGA | |
| | CTGCTACTTCAGAACAACTTTTAGATTTAGAACATTATTTAGCAA | |
| | AAACATATTGTAAAACTGCTTCATTAATGGCTAATAGTTCTCGTT | |
| | CTGTTGCAGTTCTTGCAGGTGCAGCTCCTGAAGTTTGTGATATGG | |
| | CATGGTCATACGGTCGTCATTTAGGTATTGCTTTCCAAGTAGTTG | |
| | ACGATTTATTAGATTTAACAGGTTCATCTTCTGTTTTAGGTAAAC | |
| | CTGCTTTAAACGATATGCGTTCTGGTTTAGCAACAGCACCAGTA | |
| | TTATTCGCTGCACAAGAAGAACCTGCATTACAGGCTCTTATATT | |
| | ACGTCGTTTTAAACACGACGGTGACGTAACAAAAGCAATGTCAT | |
| | TAATTGAACGTACACAAGGCTTACGTCGTGCTGAAGAACTTGCA | |
| | GCACAACACGCAAAAGCTGCTGCTGATATGATTCGTTGCTTACC | |
| | TACAGCTCAATCAGACCATGCAGAAATTGCTCGTGAAGCATTAA | |
| | TTCAAATTACACATCGTGTTTTAACACGTAAAAAAGGTACCGGT | |
| | GAAAACTTATACTTTCAAGGTTCTGGTGGTGGTGGATCAGATTA | |
| | TAAAGATGATGATGACAAAGGAACCGGTTAATCTAGACTCGAG | |
| 204 | CATATGGTACCAACTACAACATTATCATCTAACCTTAACTCACA | GPP Chimera |
| | ATTCATGCAGGTTTACGAGACTCTTAAATCAGAACTTATTCATG | |
| | ACCCATTATTTGAGTTCGATGACGATTCAAGACAATGGGTAGAA | |
| | CGTATGATTGATTATACTGTACCAGGTGGTAAAATGGTTCGTGG | |
| | TTATAGTGTAGTAGATAGTTATCAATTACTTAAAGGTGAAGAAC | |
| | TTACAGAAGAAGAGGCATTTTTAGCTTGTGCACTTGGTTGGTGT | |
| | ACAGAATGGTTTCAAGCATTCATTCTTTTACATGATGATATGATG | |
| | GATGGTAGTCACACAAGACGTGGTCAACCATGTTGGTTTCGTTT | |
| | ACCTGAGGTTGGTGCTGTTGCTATTAATGATGGTGTTTTACTTCG | |
| | TAATCACGTTCACCGTATTCTTAAAAAACATTTTCAAGGTAAAG | |
| | CATATTATGTTCATTTAGTTGATTTATTCAATGAAACTGAATTTC | |
| | AAACAATTAGTGGACAAATGATCGACTTAATTACAACATTAGTT | |
| | GGTGAAAAAGACTTATCTAAATATTCATTAAGTATTCATCGTCG | |
| | TATCGTTCAATACAAAACAGCATACTACTCATTTTACTTACCAGT | |
| | TGCTTGTGCTTTACTTATGTTTGGTGAGGATCTTGATAAACATGT | |
| | AGAAGTTAAAAATGTTCTTGTTGAAATGGGTACATATTTTCAAG | |
| | TTCAAGATGATTATTTAGATTGTTTTGGTGCTCCAGAAGTTATTG | |
| | GCAAAATTGGTACTGATATTGAAGACTTTAAATGTTCATGGTTA | |
| | GTAGTTAAAGCATTAGAATTAGCAAATGAAGAACAGAAAAAAA | |
| | CTTTACACGAAAATTATGGAAAAAAAGATCCAGCATCAGTTGCT | |
| | AAAGTTAAAGAAGTATACCACACACTTAATTTACAAGCTGTTTT | |
| | CGAAGATTATGAAGCAACATCATACAAAAAACTTATTACTTCTA | |
| | TTGAAAATCACCCATCTAAAGCTGTTCAAGCTGTTTTAAAATCTT | |
| | TCTTAGGCAAAATATACAAACGTCAAAAAGGTACCGGTGAAAA | |
| | CTTATACTTTCAAGGTTCTGGTGGCGGTGGAAGTGATTACAAAG | |
| | ATGATGACGATAAAGGAACCGGTTAATCTAGACTCGAG | |
| 205 | CATATGGTACCAAGTCAACCTTACTGGGCTGCAATTGAAGCAGA | IS-14-15 fusion |
| | CATTGAAAGATATTTAAAAAAATCAATTACAATTCGTCCACCAG | |
| | AAACTGTATTTGGTCCTATGCACCATTTAACATTTGCTGCTCCTG | |
| | CTACTGCAGCTAGTACATTATGCCTTGCTGCTTGTGAATTAGTTG | |
| | GCGGTGATCGTAGTCAAGCTATGGCAGCTGCTGCTGCTATCCAT | |
| | TTAGTTCATGCAGCTGCTTACGTTCACGAACATCTTCCTTTAACA | |
| | GATGGATCACGTCCTGTAAGTAAACCTGCTATTCAACATAAATA | |
| | TGGTCCAAACGTTGAACTTTTAACAGGTGATGGTATCGTTCCTTT | |
| | CGGTTTTGAGTTATTAGCAGGTTCAGTAGATCCAGCACGTACTG | |
| | ATGACCCTGATCGTATTTTACGTGTAATTATTGAAATTTCTCGTG | |
| | CTGGTGGACCAGAAGGCATGATTTCTGGTTTACACCGTGAGGAA | |
| | GAAATCGTAGATGGTAACACATCATTAGACTTTATAGAATATGT | |
| | ATGCAAAAAAAAATACGGTGAAATGCACGCATGTGGTGCAGCT | |
| | TGCGGAGCTATTTTAGGTGGAGCTGCTGAAGAAGAAATTCAAAA | |
| | ACTTCGTAACTTTGGTCTTTATCAAGGCACATTACGTGGTATGAT | |
| | GGAAATGAAAAATAGTCATCAGTTAATTGACGAAAATATCATTG | |
| | GAAAACTTAAAGAACTTGCTCTTGAAGAATTAGGTGGATTCCAC | |
| | GGTAAAAACGCTGAATTAATGAGTTCTTTAGTTGCTGAACCTAG | |
| | TTTATATGCAGCTTCATCAAATAACTTAGGTATCGAAGGTCGTTT | |
| | TGACTTTGACGGTTACATGCTTCGTAAAGCAAAATCTGTAAATA | |
| | AAGCATTAGAAGCTGCTGTTCAAATGAAAGAACCACTTAAAATT | |
| | CACGAATCAATGCGTTATTCATTATTAGCTGGTGGTAAACGTGTT | |
| | CGTCCAATGTTATGTATTGCAGCTTGTGAACTTGTTGGTGGTGAC | |
| | GAATCTACAGCAATGCCTGCAGCATGTGCTGTTGAAATGATTCA | |
| | CACAATGTCTTTAATGCATGATGACCTTCCATGTATGGATAACG | |
| | ATGACTTACGTCGTGGTAAACCTACAAACCACATGGCTTTTGGT | |
| | GAGTCTGTAGCTGTTCTTGCTGGTGATGCATTACTTAGTTTTGCT | |
| | TTTGAACATGTTGCTGCTGCAACAAAAGGCGCACCACCTGAACG | |
| | TATCGTACGTGTATTAGGTGAATTAGCTGTTAGTATTGGTTCAGA | |
| | AGGACTTGTAGCAGGTCAAGTTGTAGACGTTTGTTCTGAAGGCA | |
| | TGGCTGAAGTAGGATTAGATCATCTTGAATTTATTCACCATCATA | |
| | AAACTGCTGCATTATTACAAGGTTCAGTTGTTTTAGGTGCAATAT | |
| | TAGGAGGCGGTAAAGAAGAAGAAGTAGCTAAACTTCGTAAATT | |
| | TGCTAACTGTATTGGTTTACTTTTCCAAGTTGTTGATGATATTTT | |
| | AGATGTTACTAAAAGTAGTAAAGAGTTAGGTAAAACTGCAGGT | |
| | AAAGACTTAGTAGCTGATAAAACTACATATCCTAAACTTATAGG | |
| | CGTTGAAAAATCAAAAGAATTTGCTGACCGTTTAAATCGTGAAG | |
| | CACAAGAACAATTATTACATTTTCATCCTCACCGTGCTGCTCCAT | |
| | TAATCGCTTTAGCTAACTACATCGCTTACCGTGATAATGGTACCG | |
| | GTGAAAACTTATACTTCCAGGGTAGTGGTGGTGGCGGATCAGAT | |
| | TATAAAGATGACGATGATAAAGGAACCGGTTAATCTAGACTCGAG | |
| 206 | CATATGGTACCACACAAGTTCACAGGTGTTAACGCTAAATTCCA | IS-09 A118W |
| | GCAACCAGCATTAAGAAATTTATCTCCAGTGGTAGTTGAGCGCG | (G. gallus) |
| | AACGTGAGGAATTTGTAGGATTCTTTCCACAAATTGTTCGTGACT | |
| | TAACTGAAGATGGTATTGGTCATCCAGAAGTAGGTGACGCTGTA | |
| | GCTCGTCTTAAAGAAGTATTACAATACAACGCACCTGGTGGTAA | |
| | ATGCAATAGAGGTTTAACAGTTGTTGCAGCTTACCGTGAACTTT | |
| | CTGGACCAGGTCAAAAAGACGCTGAAAGTCTTCGTTGTGCTTTA | |
| | GCAGTAGGATGGTGTATTGAATTATTCCAAGCCTTTTTCTTAGTT | |
| | TGGGACGATATAATGGACCAGTCATTAACTAGACGTGGTCAATT | |
| | ATGTTGGTACAAGAAAGAAGGTGTTGGTTTAGATGCAATAAATG | |
| | ATTCTTTTCTTTTAGAAAGCTCTGTGTATCGCGTTCTTAAAAAGT | |
| | ATTGCCGTCAACGTCCATATTATGTACATTTATTAGAGCTTTTTC | |
| | TTCAAACAGCTTACCAAACAGAATTAGGACAAATGTTAGATTTA | |
| | ATCACTGCTCCTGTATCTAAGGTAGATTTAAGCCATTTCTCAGAA | |
| | GAACGTTACAAAGCTATTGTTAAGTATAAAACTGCTTTCTATTCA | |
| | TTCTATTTACCAGTTGCAGCAGCTATGTATATGGTTGGTATAGAT | |
| | TCTAAAGAAGAACATGAAAACGCAAAAGCTATTTTACTTGAGAT | |
| | GGGTGAATACTTCCAAATTCAAGATGATTATTTAGATTGTTTTGG | |
| | CGATCCTGCTTTAACAGGTAAAGTAGGTACTGATATTCAAGATA | |
| | ACAAATGTTCATGGTTAGTTGTGCAATGCTTACAAAGAGTAACA | |
| | CCAGAACAACGTCAACTTTTAGAAGATAATTACGGTCGTAAAGA | |
| | ACCAGAAAAAGTTGCTAAAGTTAAAGAATTATATGAGGCTGTAG | |
| | GTATGAGAGCCGCCTTTCAACAATACGAAGAAAGTAGTTACCGT | |
| | CGTCTTCAAGAGTTAATTGAGAAACATTCTAATCGTTTACCAAA | |
| | AGAAATTTTCTTAGGTTTAGCTCAGAAAATATACAAACGTCAAA | |
| | AAGGTACCGGTGAAAACTTATACTTTCAAGGCTCAGGTGGCGGT | |
| | GGAAGTGATTACAAAGATGATGATGATAAAGGAACCGGTTAAT | |
| | CTAGACTCGAG | |
| 207 | CATATGGTACCACACAAGTTCACAGGTGTTAACGCTAAATTCCA | FPP (G. gallus) |
| | GCAACCAGCATTAAGAAATTTATCTCCAGTGGTAGTTGAGCGCG | |
| | AACGTGAGGAATTTGTAGGATTCTTTCCACAAATTGTTCGTGACT | |
| | TAACTGAAGATGGTATTGGTCATCCAGAAGTAGGTGACGCTGTA | |
| | GCTCGTCTTAAAGAAGTATTACAATACAACGCACCTGGTGGTAA | |
| | ATGCAATAGAGGTTTAACAGTTGTTGCAGCTTACCGTGAACTTT | |
| | CTGGACCAGGTCAAAAAGACGCTGAAAGTCTTCGTTGTGCTTTA | |
| | GCAGTAGGATGGTGTATTGAATTATTCCAAGCCTTTTTCTTAGTT | |
| | GCTGACGATATAATGGACCAGTCATTAACTAGACGTGGTCAATT | |
| | ATGTTGGTACAAGAAAGAAGGTGTTGGTTTAGATGCAATAAATG | |
| | ATTCTTTTCTTTTAGAAAGCTCTGTGTATCGCGTTCTTAAAAAGT | |
| | ATTGCCGTCAACGTCCATATTATGTACATTTATTAGAGCTTTTTC | |
| | TTCAAACAGCTTACCAAACAGAATTAGGACAAATGTTAGATTTA | |
| | ATCACTGCTCCTGTATCTAAGGTAGATTTAAGCCATTTCTCAGAA | |
| | GAACGTTACAAAGCTATTGTTAAGTATAAAACTGCTTTCTATTCA | |
| | TTCTATTTACCAGTTGCAGCAGCTATGTATATGGTTGGTATAGAT | |
| | TCTAAAGAAGAACATGAAAACGCAAAAGCTATTTTACTTGAGAT | |
| | GGGTGAATACTTCCAAATTCAAGATGATTATTTAGATTGTTTTGG | |
| | CGATCCTGCTTTAACAGGTAAAGTAGGTACTGATATTCAAGATA | |
| | ACAAATGTTCATGGTTAGTTGTGCAATGCTTACAAAGAGTAACA | |
| | CCAGAACAACGTCAACTTTTAGAAGATAATTACGGTCGTAAAGA | |
| | ACCAGAAAAAGTTGCTAAAGTTAAAGAATTATATGAGGCTGTAG | |
| | GTATGAGAGCCGCCTTTCAACAATACGAAGAAAGTAGTTACCGT | |
| | CGTCTTCAAGAGTTAATTGAGAAACATTCTAATCGTTTACCAAA | |
| | AGAAATTTTCTTAGGTTTAGCTCAGAAAATATACAAACGTCAAA | |
| | AAGGTACCGGTGAAAACTTATACTTTCAAGGCTCAGGTGGCGGT | |
| | GGAAGTGATTACAAAGATGATGATGATAAAGGAACCGGTTAAT | |
| | CTAGACTCGAG | |
| | CATATGGTACCAGATTTTCCACAACAATTAGAAGCATGTGTTAA | FPP (E. coli) |
| | ACAAGCAAATCAAGCATTATCACGTTTCATCGCACCACTTCCAT | |
| | TCCAAAATACTCCTGTTGTTGAAACAATGCAATATGGTGCATTA | |
| | TTAGGAGGTAAAAGATTAAGACCATTTCTTGTATATGCAACAGG | |
| | TCACATGTTTGGAGTATCTACTAACACATTAGATGCTCCAGCTGC | |
| | TGCAGTTGAATGTATTCATGCATATAGTTTAATTCATGATGATTT | |
| | ACCTGCAATGGATGATGATGACTTAAGAAGAGGTTTACCTACAT | |
| | GTCATGTTAAATTTGGTGAAGCTAATGCTATTTTAGCTGGCGATG | |
| | CACTTCAAACTCTTGCATTCAGTATTTTATCAGATGCTGATATGC | |
| | CAGAAGTTTCAGATCGTGATCGTATTTCTATGATATCTGAATTAG | |
| | CTTCTGCTAGTGGTATTGCTGGTATGTGCGGTGGCCAAGCTCTTG | |
| | ATTTAGACGCAGAAGGAAAACACGTTCCTTTAGATGCTTTAGAG | |
| | CGTATACATCGTCACAAAACAGGAGCTTTAATTAGAGCTGCTGT | |
| | TCGTCTTGGTGCTTTATCAGCTGGAGACAAAGGTCGTCGTGCTTT | |
| | ACCAGTTTTAGACAAATACGCTGAAAGTATTGGTTTAGCTTTTCA | |
| | AGTTCAGGATGATATCTTAGATGTTGTAGGTGATACTGCTACTTT | |
| | AGGTAAACGTCAAGGTGCTGATCAACAGTTAGGCAAATCTACAT | |
| | ACCCAGCACTTTTAGGTTTAGAACAAGCTCGTAAAAAAGCAAGA | |
| | GACTTAATTGACGATGCTCGTCAAAGTCTTAAACAATTAGCAGA | |
| | ACAATCACTTGATACAAGTGCTTTAGAAGCATTAGCAGATTACA | |
| | TTATTCAACGTAATAAAGGTACCGGTGAAAATTTATATTTTCAA | |
| | GGTTCTGGTGGTGGAGGTTCAGACTATAAAGATGACGATGATAA | |
| | AGGAACCGGTTAATCTAGACTCGAG | |
| | CATATGGTACCAAGTGTTAGTTGTTGTTGTAGAAATTTAGGAAA | FPP (A. thaliana) |
| | AACTATCAAAAAAGCTATTCCAAGTCACCACTTACATTTACGTT | |
| | CTTTAGGTGGTAGTTTATATAGAAGACGTATTCAATCATCTTCAA | |
| | TGGAAACAGACTTAAAATCTACATTCTTAAATGTTTATTCAGTTC | |
| | TTAAATCAGATTTATTACACGACCCATCATTTGAATTTACAAATG | |
| | AAAGTCGTTTATGGGTAGATAGAATGCTTGATTATAATGTTCGT | |
| | GGCGGTAAACTTAATCGTGGTCTTTCTGTAGTAGACTCTTTCAAA | |
| | TTACTTAAACAAGGTAATGATTTAACTGAACAAGAAGTTTTCTT | |
| | ATCTTGTGCATTAGGTTGGTGTATTGAGTGGTTACAGGCTTACTT | |
| | TTTAGTTCTTGATGATATTATGGATAATTCAGTTACACGTCGTGG | |
| | TCAACCTTGTTGGTTTCGTGTACCACAAGTTGGTATGGTAGCTAT | |
| | TAATGATGGCATTCTTCTTCGTAACCATATTCATCGTATTCTTAA | |
| | AAAACACTTCCGTGATAAACCATATTATGTAGATTTAGTTGACC | |
| | TTTTCAATGAAGTAGAGTTACAAACTGCATGTGGACAAATGATT | |
| | GATTTAATCACAACATTTGAAGGTGAAAAAGACTTAGCTAAATA | |
| | TAGTTTATCAATTCACCGTCGTATTGTTCAATACAAAACTGCATA | |
| | TTACTCATTCTATTTACCAGTTGCATGTGCTCTTTTAATGGCTGG | |
| | CGAAAATTTAGAAAACCACATTGATGTTAAAAATGTATTAGTAG | |
| | ATATGGGTATTTACTTTCAAGTTCAGGATGATTATTTAGACTGTT | |
| | TTGCTGATCCTGAAACATTAGGTAAAATTGGCACTGATATTGAG | |
| | GACTTTAAATGTTCTTGGTTAGTTGTAAAAGCATTAGAACGTTGT | |
| | AGTGAAGAACAAACAAAAATTCTTTACGAAAACTATGGCAAAC | |
| | CTGATCCATCTAATGTTGCTAAAGTAAAAGATTTATACAAAGAA | |
| | TTAGATTTAGAAGGCGTTTTCATGGAATATGAATCTAAATCATA | |
| | CGAGAAATTAACTGGTGCTATCGAAGGTCACCAATCTAAAGCAA | |
| | TTCAAGCTGTTCTTAAATCTTTCTTAGCAAAAATCTATAAACGTC | |
| | AAAAAGGTACCGGTGAAAACTTATACTTTCAAGGTAGTGGTGGC | |
| | GGTGGTAGTGATTATAAAGATGATGATGATAAAGGAACCGGTTA | |
| | ATCTAGACTCGAG | |
| | CATATGGTACCAGCTGATCTTAAATCAACATTCTTAGATGTTTAT | FPP (A. thaliana) |
| | TCAGTATTAAAAAGTGATTTATTACAAGATCCATCTTTTGAATTT | |
| | ACACACGAAAGTCGTCAATGGTTAGAACGTATGTTAGATTATAA | |
| | TGTTCGTGGAGGCAAATTAAACAGAGGTTTAAGTGTAGTAGACA | |
| | GTTACAAACTTTTAAAACAAGGTCAAGACTTAACAGAAAAAGA | |
| | AACATTTTTATCTTGTGCTTTAGGTTGGTGTATTGAATGGTTACA | |
| | AGCATACTTCTTAGTTTTAGACGATATTATGGATAATTCTGTAAC | |
| | TAGACGTGGTCAACCATGTTGGTTTCGTAAACCAAAAGTAGGTA | |
| | TGATTGCTATTAATGATGGAATACTTCTTCGTAACCACATTCATC | |
| | GTATTCTTAAAAAACACTTTCGTGAAATGCCTTATTATGTAGACC | |
| | TTGTAGACTTATTTAACGAAGTAGAATTTCAAACAGCTTGTGGT | |
| | CAAATGATTGACTTAATTACAACATTTGATGGTGAAAAAGACCT | |
| | TTCAAAATATTCACTTCAGATTCACCGTCGTATTGTTGAGTACAA | |
| | AACAGCATACTACTCTTTCTATTTACCTGTAGCATGTGCTTTACT | |
| | TATGGCAGGTGAAAATTTAGAAAATCACACAGATGTTAAAACTG | |
| | TATTAGTTGATATGGGTATCTATTTCCAAGTTCAAGATGATTATT | |
| | TAGATTGCTTCGCTGATCCAGAAACATTAGGTAAAATTGGTACA | |
| | GATATTGAAGACTTTAAATGTAGTTGGTTAGTAGTAAAAGCATT | |
| | AGAACGTTGTAGTGAAGAACAAACAAAAATTCTTTACGAAAATT | |
| | ATGGAAAAGCTGAACCTTCAAATGTAGCTAAAGTTAAAGCATTA | |
| | TACAAAGAATTAGATTTAGAGGGTGCATTTATGGAATATGAAAA | |
| | AGAATCATACGAGAAACTTACAAAACTTATTGAAGCACATCAAT | |
| | CAAAAGCTATTCAAGCAGTTCTTAAATCTTTCTTAGCTAAAATTT | |
| | ATAAACGTCAAAAAGGTACCGGTGAAAACTTATACTTTCAAGGC | |
| | TCTGGAGGTGGTGGTTCAGACTATAAAGATGATGATGATAAAGG | |
| | AACCGGTTAATCTAGACTCGAG | |
| | CATATGGTACCAAGTGGCGAACCTACTCCAAAAAAAATGAAAG | FPP (C. reinhardtii) |
| | CAACATACGTTCACGACCGTGAAAACTTTACAAAAGTATACGAA | |
| | ACTCTTCGTGACGAATTACTTAACGATGATTGTCTTAGTCCAGCT | |
| | GGTTCACCTCAGGCTCAAGCTGCTCAAGAGTGGTTTAAAGAAGT | |
| | TAATGATTATAATGTTCCTGGTGGAAAACTTAACCGTGGTATGG | |
| | CTGTATATGACGTTTTAGCTTCAGTTAAAGGTCCAGATGGTTTAA | |
| | GTGAAGACGAAGTATTTAAAGCTAACGCTCTTGGTTGGTGTATT | |
| | GAGTGGTTACAAGCATTTTTCTTAGTTGCTGATGATATAATGGAT | |
| | GGTTCAATTACACGTCGTGGCCAACCTTGTTGGTACAAACAACC | |
| | TAAAGTTGGTATGATTGCTTGTAATGATTACATCTTATTAGAATG | |
| | CTGTATTTACTCAATTCTTAAAAGACATTTTAGAGGTCACGCTGC | |
| | ATACGCTCAACTTATGGACCTTTTCCATGAAACTACATTCCAGAC | |
| | TTCACACGGTCAATTATTAGATTTAACAACAGCACCTATCGGTTC | |
| | TGTAGACTTATCAAAATATACAGAAGATAATTACCTTCGTATTG | |
| | TAACATATAAAACTGCATACTATTCTTTTTATTTACCTGTAGCAT | |
| | GTGGTATGGTATTAGCTGGCATTACAGATCCAGCTGCTTTTGATC | |
| | TTGCAAAAAATATTTGTGTTGAAATGGGTCAATATTTCCAGATTC | |
| | AAGACGATTATTTAGATTGCTATGGTGACCCTGAGGTTATTGGT | |
| | AAAATCGGTACAGACATAGAAGACAACAAATGTAGTTGGTTAG | |
| | TTTGCACAGCTCTTAAAATCGCAACAGAAGAACAAAAAGAGGTT | |
| | ATAAAAGCTAATTATGGTCACAAAGAGGCTGAATCAGTAGCAG | |
| | CAATTAAAGCATTATACGTTGAATTAGGTATTGAACAACGTTTT | |
| | AAAGACTATGAAGCTGCATCATACGCAAAATTAGAAGGTACAA | |
| | TTAGTGAACAAACTTTATTACCTAAAGCAGTATTTACTTCTTTAT | |
| | TAGCTAAAATCTATAAAAGAAAAAAAGGTACCGGTGAGAACTT | |
| | ATACTTTCAAGGTAGTGGAGGTGGTGGTTCAGACTATAAAGATG | |
| | ATGATGATAAAGGAACCGGTTAATCTAGACTCGAG | |
| | CATATGGTACCAGTAACAGCAGCACGTGCAACACCAAAATTAA | Geranylgeranyl |
| | GTAATAGAAAATTACGTGTTGCTGTAATTGGAGGCGGTCCAGCA | reductase (A. thaliana) |
| | GGAGGTGCAGCTGCTGAAACATTAGCACAAGGAGGTATTGAAA | |
| | CAATTCTTATCGAACGTAAAATGGATAATTGTAAACCATGTGGT | |
| | GGTGCTATTCCATTATGTATGGTAGGAGAGTTCAATTTACCTTTA | |
| | GACATTATTGACCGTCGTGTAACAAAAATGAAAATGATCTCTCC | |
| | TTCAAACATTGCAGTTGATATCGGTCGTACACTTAAAGAACACG | |
| | AATATATTGGTATGGTTCGTCGTGAGGTACTTGATGCTTATCTTC | |
| | GTGAACGTGCAGAAAAATCAGGTGCTACTGTTATTAACGGTTTA | |
| | TTCTTAAAAATGGATCACCCAGAAAATTGGGATTCACCATATAC | |
| | ACTTCACTACACAGAGTATGATGGAAAAACAGGTGCTACAGGA | |
| | ACTAAAAAAACTATGGAAGTAGATGCTGTTATTGGTGCTGATGG | |
| | TGCTAATTCTCGTGTTGCAAAAAGTATTGACGCAGGTGATTATG | |
| | ATTATGCTATTGCATTTCAAGAACGTATTCGTATACCTGATGAGA | |
| | AAATGACTTATTATGAGGACTTAGCTGAGATGTATGTAGGTGAT | |
| | GATGTATCACCAGACTTCTACGGTTGGGTATTCCCAAAATGTGA | |
| | TCATGTAGCTGTTGGTACAGGTACTGTAACACATAAAGGTGATA | |
| | TCAAAAAATTCCAGTTAGCTACACGTAATCGTGCTAAAGATAAA | |
| | ATTCTTGGTGGCAAAATAATCCGTGTAGAGGCTCATCCTATTCC | |
| | AGAGCATCCTAGACCACGTCGTTTATCAAAACGTGTTGCATTAG | |
| | TAGGCGACGCAGCAGGTTACGTTACTAAATGTTCAGGAGAAGG | |
| | AATTTACTTCGCAGCTAAATCTGGTCGTATGTGTGCTGAAGCTAT | |
| | CGTTGAAGGTTCACAAAATGGCAAAAAAATGATAGATGAAGGC | |
| | GATTTAAGAAAATACTTAGAAAAATGGGATAAAACTTACTTACC | |
| | AACTTATCGTGTTTTAGATGTACTTCAAAAAGTTTTCTATCGTTC | |
| | TAACCCAGCTCGTGAGGCTTTTGTTGAAATGTGTAACGATGAGT | |
| | ATGTACAGAAAATGACATTTGATTCTTACCTTTATAAACGTGTA | |
| | GCTCCTGGTAGTCCATTAGAAGATATCAAATTAGCTGTAAATAC | |
| | TATTGGTTCACTTGTTCGTGCTAACGCATTACGTCGTGAAATTGA | |
| | GAAATTATCAGTAGGTACCGGTGAGAATCTTTACTTTCAAGGAT | |
| | CAGGTGGTGGTGGTTCTGATTATAAAGATGACGATGATAAAGGA | |
| | ACCGGTTAATCTAGACTCGAG | |
| | CATATGGTACCAGTAGCTGTTATTGGTGGTGGTCCAAGTGGCGC | Geranylgeranyl |
| | TTGTGCAGCAGAAACTTTAGCAAAAGGTGGTGTAGAAACTTTCT | reductase (C. reinhardtii) |
| | TACTTGAGCGTAAATTAGATAATTGTAAACCTTGTGGAGGTGCA | |
| | ATTCCATTATGTATGGTTGAAGAATTTGATTTACCAATGGAAAT | |
| | AATTGACCGTCGTGTTACTAAAATGAAAATGATATCACCTTCAA | |
| | ACCGTGAAGTTGATGTTGGAAAAACTTTATCAGAAACTGAATGG | |
| | ATCGGTATGTGTCGTCGTGAAGTATTTGACGATTACTTAAGAAA | |
| | CCGTGCACAGAAATTAGGTGCTAATATTGTTAACGGTTTATTCAT | |
| | GCGTTCAGAACAACAATCTGCAGAGGGTCCATTCACAATTCACT | |
| | ATAATTCTTATGAAGACGGTAGTAAAATGGGAAAACCTGCTACT | |
| | TTAGAAGTTGATATGATAATTGGTGCAGATGGAGCAAATTCTCG | |
| | TATTGCAAAAGAGATAGATGCAGGTGAATACGACTACGCTATAG | |
| | CTTTTCAAGAACGTATTCGTATTCCTGATGATAAAATGAAATATT | |
| | ACGAAAACCTTGCTGAAATGTATGTAGGTGATGACGTATCTCCT | |
| | GATTTCTATGGTTGGGTTTTTCCTAAATATGATCACGTTGCTGTT | |
| | GGTACAGGTACTGTTGTAAACAAAACAGCTATTAAACAATATCA | |
| | ACAGGCAACACGTGACAGATCAAAAGTTAAAACAGAAGGTGGC | |
| | AAAATTATACGTGTTGAAGCACACCCAATTCCAGAACATCCACG | |
| | TCCACGTCGTTGTAAAGGTCGTGTTGCATTAGTAGGCGACGCAG | |
| | CTGGTTATGTTACAAAATGTTCTGGCGAGGGCATTTACTTTGCTG | |
| | CTAAATCTGGTAGAATGGCTGCTGAAGCTATTGTAGAAGGTTCT | |
| | GCTAACGGTACAAAAATGTGTGGTGAGGATGCAATTCGTGTTTA | |
| | TTTAGATAAATGGGATCGTAAATATTGGACAACATACAAAGTAT | |
| | TAGACATTTTACAAAAAGTATTTTATCGTAGTAATCCAGCACGT | |
| | GAAGCATTTGTTGAATTATGTGAAGATAGTTATGTACAGAAAAT | |
| | GACATTTGATTCATACTTATATAAAACTGTTGTTCCAGGAAACCC | |
| | ATTAGACGACGTAAAATTACTTGTTCGTACAGTATCTTCTATTTT | |
| | ACGTTCAAATGCTTTACGTTCTGTTAATTCTAAATCTGTAAATGT | |
| | TTCTTTCGGCTCTAAAGCAAATGAGGAACGTGTTATGGCTGCAG | |
| | GTACCGGTGAAAATCTTTATTTTCAAGGTTCAGGAGGTGGTGGT | |
| | TCAGATTATAAAGATGATGATGACAAAGGAACCGGTTAATCTAG | |
| | ACTCGAG | |
| | CATATGGTACCAGCAATGGCAGTACCATTAGATGTAGTAATTAC | Chlorophyllido- |
| | ATATCCTTCTTCAGGTGCTGCTGCTTATCCAGTACTTGTTATGTA | hydrolase (C. reinhardtii) |
| | TAACGGTTTCCAAGCTAAAGCTCCATGGTATCGTGGTATTGTAG | |
| | ATCATGTTTCTAGTTGGGGTTACACAGTTGTTCAATATACAAATG | |
| | GTGGCTTATTTCCTATTGTTGTAGATCGTGTTGAGTTAACTTATT | |
| | TAGAGCCATTATTAACTTGGTTAGAAACACAAAGTGCTGATGCT | |
| | AAATCTCCTTTATACGGTCGTGCAGATGTTTCTCGTTTAGGTACA | |
| | ATGGGTCATTCACGTGGTGGTAAATTAGCAGCTTTACAATTTGCT | |
| | GGACGTACAGATGTAAGTGGTTGTGTATTATTTGACCCTGTAGA | |
| | TGGAAGTCCAATGACACCAGAATCTGCTGATTATCCTTCAGCTA | |
| | CAAAAGCATTAGCAGCAGCTGGTCGTTCTGCTGGCTTAGTAGGT | |
| | GCAGCTATTACAGGTTCATGTAATCCAGTAGGTCAAAATTACCC | |
| | AAAATTCTGGGGTGCTTTAGCTCCTGGTTCTTGGCAAATGGTATT | |
| | ATCACAAGCTGGTCACATGCAATTTGCTCGTACTGGTAATCCATT | |
| | CTTAGATTGGTCATTAGACCGTTTATGTGGTCGTGGTACAATGAT | |
| | GAGTTCAGATGTTATTACATATAGTGCAGCATTTACTGTTGCTTG | |
| | GTTTGAAGGTATTTTTCGTCCTGCTCAAAGTCAAATGGGTATTTC | |
| | TAATTTCAAAACTTGGGCTAATACTCAAGTTGCAGCTCGTAGTA | |
| | TCACTTTTGATATTAAACCTATGCAATCTCCTCAGGGTACCGGTG | |
| | AAAACCTTTACTTTCAAGGTAGTGGTGGTGGAGGAAGTGATTAT | |
| | AAAGATGATGATGACAAAGGAACCGGTTAATCTAGACTCGAG | |
| | CATATGGTACCAGCACCACCAAAACCAGTTCGTATAACTTGTCC | Chlorophyllido- |
| | AACAGTAGCTGGCACTTATCCTGTTGTTTTATTCTTTCACGGTTT | hydrolase (A. thaliana) |
| | TTATCTTCGTAACTATTTCTATTCAGATGTTTTAAATCATATTGCT | |
| | AGTCATGGTTACATCTTAGTTGCACCACAATTATGTAAACTTTTA | |
| | CCTCCAGGTGGCCAAGTAGAAGTTGATGACGCTGGTTCAGTTAT | |
| | TAACTGGGCTTCAGAGAATCTTAAAGCACACCTTCCAACTTCTG | |
| | TTAATGCTAATGGTAAATATACATCTTTAGTTGGACATTCACGTG | |
| | GTGGCAAAACAGCTTTCGCAGTTGCATTAGGTCACGCAGCTACA | |
| | TTAGATCCATCAATTACATTTTCAGCATTAATTGGTATTGATCCA | |
| | GTAGCAGGAACTAACAAATACATTCGTACAGATCCACACATCTT | |
| | AACTTATAAACCTGAATCATTTGAATTAGATATTCCTGTAGCTGT | |
| | TGTAGGCACTGGTCTTGGTCCAAAATGGAATAACGTAATGCCTC | |
| | CATGCGCACCTACAGATTTAAACCACGAAGAATTTTACAAAGAA | |
| | TGTAAAGCTACTAAAGCTCACTTTGTTGCTGCTGATTATGGTCAC | |
| | ATGGACATGTTAGACGACGATCTTCCAGGTTTTGTAGGCTTCAT | |
| | GGCTGGTTGTATGTGTAAAAATGGTCAACGTAAAAAATCAGAAA | |
| | TGCGTTCTTTTGTAGGTGGTATAGTTGTAGCATTCTTAAAATATT | |
| | CTTTATGGGGTGAAAAAGCTGAAATAAGATTAATTGTTAAAGAT | |
| | CCTAGTGTATCTCCTGCTAAATTAGACCCATCACCAGAATTAGA | |
| | AGAAGCATCAGGTATTTTTGTTGGTACCGGTGAAAATCTTTATTT | |
| | TCAAGGTTCAGGTGGAGGTGGTTCTGATTATAAAGATGATGATG | |
| | ACAAAGGAACCGGTTAATCTAGACTCGAG | |
| | CATATGGTACCAGCTACACCAGTTGAAGAAGGTGATTATCCAGT | Chlorophyllido- |
| | TGTAATGTTATTACATGGCTACCTTTTATATAATTCATTTTATTCA | hydrolase (A. thaliana) |
| | CAATTAATGTTACATGTATCATCTCACGGTTTCATCTTAATTGCT | |
| | CCACAATTATACTCAATTGCTGGTCCTGATACTATGGATGAAATT | |
| | AAAAGTACTGCTGAGATTATGGACTGGTTATCAGTTGGTTTAAA | |
| | TCACTTTTTACCAGCTCAAGTTACACCTAATTTATCTAAATTTGC | |
| | ATTATCTGGTCATAGTCGTGGTGGTAAAACTGCTTTTGCTGTAGC | |
| | ATTAAAAAAATTTGGTTATTCTTCAAACTTAAAAATTAGTACTTT | |
| | AATTGGTATTGATCCAGTAGACGGAACAGGTAAAGGTAAACAA | |
| | ACTCCACCTCCTGTTTTAGCATATTTACCTAATAGTTTTGACTTA | |
| | GACAAAACACCAATTTTAGTAATTGGTTCAGGTTTAGGTGAAAC | |
| | TGCACGTAATCCTTTATTTCCTCCATGTGCTCCTCCAGGTGTTAA | |
| | CCACCGTGAGTTTTTCCGTGAATGTCAAGGTCCAGCATGGCACT | |
| | TTGTTGCTAAAGATTATGGTCATTTAGACATGCTTGATGATGATA | |
| | CAAAAGGTATTCGTGGCAAATCTAGTTACTGTTTATGCAAAAAT | |
| | GGTGAAGAACGTCGTCCAATGCGTCGTTTCGTTGGTGGTTTAGTT | |
| | GTTAGTTTTCTTAAAGCATATCTTGAAGGTGATGATCGTGAATTA | |
| | GTAAAAATCAAAGATGGTTGTCATGAAGATGTACCTGTTGAAAT | |
| | TCAAGAATTTGAAGTAATTATGGGTACCGGTGAAAATCTTTACT | |
| | TTCAAGGTTCAGGCGGTGGAGGTTCAGATTATAAAGATGATGAT | |
| | GACAAAGGAACCGGTTAATCTAGACTCGAG | |
| | CATATGGTACCAGCTGCTGCTGCACCTGCTGAGACAATGAATAA | Chlorophyllido- |
| | ATCTGCAGCTGGCGCTGAAGTACCAGAGGCTTTCACATCAGTTT | hydrolase (T. Aestivum) |
| | TTCAACCAGGTAAATTAGCAGTTGAAGCAATTCAAGTAGATGAA | |
| | AATGCAGCTCCTACTCCACCTATTCCTGTTTTAATAGTTGCTCCA | |
| | AAAGATGCTGGTACATATCCAGTTGCTATGTTATTACACGGATTT | |
| | TTCTTACATAATCACTTTTATGAACACTTATTACGTCACGTTGCA | |
| | TCTCATGGCTTTATCATTGTTGCTCCACAATTTTCTATTAGTATTA | |
| | TTCCATCAGGAGATGCTGAAGACATCGCTGCTGCTGCAAAAGTA | |
| | GCAGATTGGTTACCTGACGGATTACCAAGTGTTTTACCAAAAGG | |
| | TGTTGAACCAGAGTTATCAAAACTTGCTTTAGCTGGACACAGTC | |
| | GTGGTGGTCACACAGCTTTTTCTTTAGCTTTAGGTCACGCTAAAA | |
| | CACAATTAACTTTCAGTGCATTAATTGGTTTAGATCCTGTTGCTG | |
| | GAACAGGTAAATCATCTCAATTACAACCAAAAATTCTTACTTAT | |
| | GAGCCAAGTTCATTTGGTATGGCTATGCCAGTTTTAGTTATTGGT | |
| | ACAGGTTTAGGAGAAGAAAAAAAAAACATTTTCTTTCCTCCATG | |
| | TGCTCCTAAAGACGTAAACCATGCAGAATTTTATCGTGAATGTA | |
| | GACCACCATGTTACTATTTTGTAACTAAAGATTATGGCCATCTTG | |
| | ATATGTTAGATGATGACGCTCCAAAATTTATCACATGTGTTTGTA | |
| | AAGACGGTAATGGATGTAAAGGAAAAATGCGTCGTTGTGTAGCT | |
| | GGCATCATGGTTGCTTTCTTAAACGCTGCTTTAGGTGAAAAAGA | |
| | CGCAGATTTAGAAGCTATTTTACGTGATCCAGCAGTTGCTCCTAC | |
| | AACATTAGACCCAGTTGAACACCGTGTTGCTGGTACCGGTGAGA | |
| | ATTTATACTTCCAGGGATCTGGTGGTGGTGGCAGTGATTATAAA | |
| | GATGATGATGATAAAGGAACCGGTTAATCTAGACTCGAG | |
| | CATATGGTACCAAGTCACAAAAAAAAAAACGTAATCTTCTTCGT | Phosphatase (S. cerevisiae) |
| | AACTGATGGTATGGGTCCTGCTTCTCTTTCAATGGCTCGTTCATT | |
| | TAATCAACACGTTAATGATTTACCAATTGATGATATTTTAACATT | |
| | AGATGAACATTTTATTGGAAGTTCAAGAACACGTTCATCAGATT | |
| | CACTTGTAACTGACTCAGCTGCTGGAGCTACAGCTTTTGCTTGTG | |
| | CACTTAAATCATACAATGGTGCTATAGGTGTAGATCCACACCAT | |
| | CGTCCATGTGGAACTGTTTTAGAAGCTGCTAAATTAGCAGGTTA | |
| | TTTAACAGGATTAGTAGTTACTACACGTATTACTGATGCTACACC | |
| | AGCTAGTTTCTCAAGTCACGTAGATTATCGTTGGCAAGAAGATT | |
| | TAATTGCAACACACCAATTAGGTGAATATCCTTTAGGACGTGTT | |
| | GTTGATCTTCTTATGGGTGGTGGTCGTTCTCACTTTTATCCTCAA | |
| | GGTGAAAAAGCTAGTCCATACGGTCACCACGGTGCACGTAAAG | |
| | ATGGTCGTGATTTAATCGATGAAGCTCAAAGTAATGGCTGGCAG | |
| | TATGTAGGAGATCGTAAAAATTTTGATTCTTTACTTAAATCACAT | |
| | GGTGAAAATGTTACTTTACCATTTTTAGGTTTATTTGCTGACAAC | |
| | GATATCCCATTTGAAATTGATCGTGATGAAAAAGAATATCCTAG | |
| | TTTAAAAGAACAAGTAAAAGTAGCATTAGGTGCTTTAGAAAAA | |
| | GCAAGTAACGAAGATAAAGATAGTAATGGTTTCTTTTTAATGGT | |
| | AGAAGGTTCTCGTATTGATCATGCTGGCCATCAAAACGATCCTG | |
| | CATCTCAAGTACGTGAAGTATTAGCATTTGATGAGGCTTTTCAAT | |
| | ATGTATTAGAATTTGCAGAAAACAGTGATACAGAAACAGTATTA | |
| | GTAAGTACATCAGATCATGAAACAGGTGGTTTAGTTACTTCAAG | |
| | ACAAGTAACAGCATCATACCCACAATATGTATGGTATCCTCAAG | |
| | TATTAGCTAACGCTACACATAGTGGAGAGTTTCTTAAACGTAAA | |
| | TTAGTTGATTTCGTTCATGAACACAAAGGCGCATCATCAAAAAT | |
| | AGAAAACTTCATAAAACACGAAATTCTTGAAAAAGATTTAGGTA | |
| | TTTATGATTATACAGATTCTGACTTAGAAACACTTATTCATTTAG | |
| | ATGATAACGCTAATGCAATTCAAGATAAACTTAATGATATGGTA | |
| | AGTTTTAGAGCTCAAATTGGTTGGACAACACATGGTCATTCAGC | |
| | AGTTGATGTAAACATATATGCTTACGCAAACAAAAAAGCTACAT | |
| | GGTCTTATGTTCTTAATAACTTACAAGGTAATCACGAAAACACA | |
| | GAAGTTGGTCAATTCTTAGAGAATTTCTTAGAATTAAACTTAAA | |
| | TGAAGTTACTGATTTAATCCGTGATACAAAACATACTTCTGATTT | |
| | TGACGCAACAGAAATAGCAAGTGAGGTTCAACACTATGATGAA | |
| | TATTACCACGAATTAACAAATGGTACCGGTGAAAATCTTTATTTT | |
| | CAAGGTTCTGGTGGAGGTGGCAGTGATTATAAAGATGATGATGA | |
| | CAAAGGAACCGGTTAATCTAGACTCGAG | |
| | CATATGGTACCAGCTTTATACGACATTATTAACTATTTCTACGGT | Phosphatase (C. albicans) |
| | TCAAACTCTAAATTCAACCGTATTACATGGGGTTTTAAATCACC | |
| | AACTTTCATCAAATGGAGAATTACTGATTTCATTTTAATCATCGT | |
| | TTTAATTGTTCTTTTCTTCGTAACTTCTCAAGCAGAGCCATTCCA | |
| | TCGTCAATTTTATCTTAACGACATGACTATCCAACATCCTTTTGC | |
| | AGAACATGAACGTGTAACTAATATTCAACTTGGTTTATATTCAA | |
| | CAGTAATTCCTTTATCAGTTATTATCATTGTTGCTTTAATTAGTA | |
| | CATGTCCACCTAAATACAAATTATACAACACTTGGGTTTCAAGT | |
| | ATTGGTTTACTTTTATCAGTTTTAATCACATCTTTTGTTACAAAC | |
| | ATCGTTAAAAACTGGTTTGGACGTTTACGTCCTGACTTCTTAGAT | |
| | CGTTGCCAACCAGCTAACGATACACCTAAAGATAAATTAGTTTC | |
| | TATTGAGGTTTGTACTACAGACAATTTAGACCGTTTAGCTGACG | |
| | GTTTTCGTACAACACCTTCTGGTCATTCTTCAATCTCATTTGCTG | |
| | GTTTATTCTATTTAACATTATTTCTTTTAGGTCAATCTCAGGCAA | |
| | ATAATGGTAAAACATCTTCATGGCGTACAATGATCAGTTTTATA | |
| | CCTTGGTTAATGGCTTGTTATATCGCTTTAAGTCGTACACAAGAC | |
| | TACCGTCATCATTTCATTGACGTATTTGTTGGTAGTTGCTTAGGC | |
| | TTAATTATCGCAATTTGGCAATACTTCCGTTTATTCCCTTGGTTC | |
| | GGTGGTAACCAAGCAAATGATTCATTTAACAACCGTATTATGAT | |
| | TGAAGAGATTAAACGTAAAGAGGAAATTAAACAAGATGAAAAT | |
| | AACTACCGTCGTATTTCTGATATTTCTACTAATGTAGGTACCGGT | |
| | GAAAACCTTTACTTTCAAGGTTCAGGTGGCGGCGGTTCAGATTA | |
| | TAAAGATGATGACGACAAAGGAACCGGTTAATCTAGACTCGAG | |

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of yeast genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Where a range of values is provided, it is understood that each intervening value, between the upper and lower limits of that range is also specifically disclosed. Each smaller range between any stated value or intervening value in a stated range and any other stated or intervening value in that stated range is encompassed. The upper and lower limits of these smaller ranges can independently be included or excluded in the range, and each range where either, neither or both limits are included in the smaller ranges is also encompassed, subject to any specifically excluded limit in the stated range. Where the stated range includes one or both of the limits, ranges excluding either or both of those included limits are also included.

1. An isolated vector comprising: (a) a nucleic acid encoding an enzyme that produces an isoprenoid with two phosphates; and (b) a promoter configured for expression of said nucleic acid in a chloroplast of a non-vascular, photosynthetic organism, wherein the vector does not comprise the entire genome of a chloroplast.

2. The vector of claim 1, wherein said isoprenoid with two phosphates is GPP, IPP, FPP, GGPP or DMAPP.

3-4. (canceled)

5. The vector of claim 1, further comprising a nucleic acid encoding a second enzyme which modifies the isoprenoid with two phosphates.

6. The vector of claim 5, wherein said second enzyme is botyrococcene synthase, limonene synthase, cineole synthase, pinene synthase, camphene synthase, sabinene synthase, myrcene synthase, abietadiene synthase, taxadiene synthase, bisabolene synthase, diapophytoene desaturase, diapophytoene synthase, monoterpene synthase, terpinolene synthase, zingiberene synthase, ocimene synthase, sesquiterpene synthase, curcumene synthase, farnesene synthase, geranylgeranyl reductase, chlorophyllidohydrolase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, gamma-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase.

7-8. (canceled)

9. The vector of claim 1, wherein said chloroplast is from a microalga.

10. The vector of claim 9, wherein said microalga is C. reinhardtii, D. salina, H. pluvalis, S. dimorphus, D. viridis, or D. tertiolecta.

11. The vector of claim 1, comprising any of the sequences in Table 5 or a sequence having at least 70% identity thereto.

12-31. (canceled)

32. A genetically modified chloroplast comprising the vector of claim 1.

33. A non-vascular, photosynthetic organism comprising the chloroplast of claim 32.

34. A method of producing an isoprenoid comprising: (a) transforming a chloroplast of a non-vascular, photosynthetic organism with a nucleic acid encoding an enzyme that produces an isoprenoid with two phosphates, and; (b) collecting at least one isoprenoid produced by said transformed organism.

35. The method of claim 34, further comprising growing said organism in an aqueous environment, wherein CO₂ is supplied to said organism.

36. The method of claim 35, wherein said CO₂ is at least partially derived from a burned fossil fuel.

37. (canceled)

38. The method of claim 34, wherein said isoprenoid with two phosphates is GPP, IPP, FPP, GGPP or DMAPP.

39. The method of claim 34, wherein said collecting step comprises one or more of the following steps: (a) harvesting said transformed organism; (b) harvesting said isoprenoid from a cell medium; (c) mechanically disrupting said organism; or (d) chemically disrupting said organism.

40-41. (canceled)

42. A method for producing an isoprenoid comprising: (a) transforming the chloroplast of a non-vascular, photosynthetic organism to produce said isoprenoid, wherein said organism is not transformed with isoprene synthase or a methyl-butenol synthase; and (b) collecting said isoprenoid.

43. The method of claim 42, further comprising growing said organism in an aqueous environment, wherein CO₂ is supplied to said organism.

44. The method of claim 43, wherein said CO₂ is at least partially derived from a burned fossil fuel.

45. (canceled)

46. The method of claim 42, wherein isoprenoid is GPP, IPP, FPP, GGPP or DMAPP.

47. The method of claim 42, wherein said chloroplast is transformed with a nucleic acid encoding botyrococcene synthase, limonene synthase, cineole synthase, pinene synthase, camphene synthase, sabinene synthase, myrcene synthase, abietadiene synthase, taxadiene synthase, bisabolene synthase, diapophytoene desaturase, diapophytoene synthase, monoterpene synthase, terpinolene synthase, zingiberene synthase, ocimene synthase, sesquiterpene synthase, curcumene synthase, farnesene synthase, geranylgeranyl reductase, chlorophyllidohydrolase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinenesynthase, β-phellandrene synthase, terpinolene synthase, (+)-3-carene synthase, syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, gamma-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase.

48. The method of claim 42, wherein said collecting step comprises one or more of the following steps: (a) harvesting said transformed organism; (b) harvesting said isoprenoid from a cell medium; (d) mechanically disrupting said organism; or (e) chemically disrupting said organism.

49-66. (canceled)

67. The organism of claim 33, wherein said organism comprises: a first nucleic acid encoding a botryococcene synthase, and a second nucleic acid encoding an FPP synthase.

68. The host cell of claim 67, wherein said first and second nucleic acids are integrated into a chloroplast genome.

69-127. (canceled)

128. The method of claim 34, wherein phytol production in said non-vascular photosynthetic organism is increased above a level produced by said organism not containing said nucleic acid.

129. The method of claim 128, wherein said nucleic acid encodes an enzyme selected from the group consisting of a GPP synthase, a FPP synthase, a geranylgeranyl reductase, a chlorophyllidohydrolase, and a pyrophosphatase.

130. (canceled)

131. The method of claim 129, wherein said enzyme is endogenous to said organism or is homologous to an endogenous enzyme of said organism.

132. The method of claim 131 wherein said enzyme is overexpressed.

133. The method of claim 129, wherein said enzyme is exogenous to said organism.

134. (canceled)

135. The method of claim 128, further comprising transformation of said organism with a nucleic acid which results in production of dimethylallyl alcohol, isopentyl alcohol, geraniol, farnesol or geranylgeraniol.

136-137. (canceled)

138. The method of claim 128, wherein said nucleic acid comprises a sequence from Table 7 or a sequence with 70% homology thereto.

139. A host cell comprising an introduced nucleic acid, wherein said nucleic acid results in an increase in production of phytol by said host cell above a level produced by a host cell not containing said nucleic acid, and wherein said host cell is a non-vascular photosynthetic organism.

140-142. (canceled)

143. The host cell of claim 139, wherein said nucleic acid is present in a chloroplast.

144. The host cell of claim 139, wherein said nucleic acid encodes an enzyme selected from the group consisting of a GPP synthase, a FPP synthase, a geranylgeranyl reductase, a chlorophyllidohydrolase, and a pyrophosphatase.

145. The host cell of claim 139, further comprising a nucleic acid which results in production of dimethylallyl alcohol, isopentyl alcohol, geraniol, farnesol or geranylgeraniol.

146-147. (canceled)

148. The method of claim 128, further comprising:
collecting said phytol from said organism.

149. The method of claim 148, wherein said nucleic acid encodes an enzyme selected from the group consisting of a GPP synthase, a FPP synthase, a geranylgeranyl reductase, a chlorophyllidohydrolase, and a pyrophosphatase.

150. (canceled)

151. The method of claim 148, wherein said nucleic acid encodes an enzyme endogenous to said organism or an enzyme homologous to an endogenous enzyme of said organism.

152-154. (canceled)

155. The method of claim 148, further comprising transformation of said organism with a nucleic acid which results in production of dimethylallyl alcohol, isopentyl alcohol, geraniol, farnesol or geranylgeraniol.

156-163. (canceled)


SEQUENCE LISTING (209 sequences)

SEQ ID NO: 1 - protein, 572 aa, Artificial Sequence
  Description of Artificial Sequence: Synthetic polypeptide
MVPRRSGNYNPSRWDVNFIQSLLSDYKEDKHVIRASELVTLVKMELEKETDQIRQLELIDDLQRMGLSDHFQNEFKEILSSIYLDHHYYKNPFPKEERDLYSTSLAFRLLREHGFQVAQEVFDSFKNEEGEFKESLSDDTRGLLQLYEASFLLTEGETTLESAREFATKFLEEKVNEGGVDGDLLTRIAYSLDIPLHWRIKRPNAPVWIEWYRKRPDMNPVVLELAILDLNIVQAQFQEELKESFRWWRNTGFVEKLPFARDRLVECYFWNTGIIEPRQHASARIMMGKVNALITVIDDIYDVYGTLEELEQFTDLIRRWDINSIDQLPDYMQLCFLALNNFVDDTSYDVMKEKGVNVIPYLRQSWVDLADKYMVEARWFYGGHKPSLEEYLENSWQSISGPCMLTHIFFRVTDSFTKETVDSLYKYHDLVRWSSFVLRLADDLGTSVEEVSRGDVPKSLQCYMSDYNASEAEARKHVKWLIAEVWKKMNAERVSKDSPFGKDFIGCAVDLGRMAQLMYHNGDGHGTQHPIIHQQMTRTLFEPFAGTGENLYFQGSGGGGSDYKDDDDKGTG

SEQ ID NO: 2 - protein, 565 aa, Artificial Sequence
  Description of Artificial Sequence: Synthetic polypeptide
MVPRRTGGYQPTLWDFSTIQLFDSEYKEEKHLMRAAGMIAQVNMLLQEEVDSIQRLELIDDLRRLGISCHFDREIVEILNSKYYTNNEIDESDLYSTALRFKLLRQYDFSVSQEVFDCFKNDKGTDFKPSLVDDTRGLLQLYEASFLSAQGEETLHLARDFATKFLHKRVLVDKDINLLSSIERALELPTHWRVQMPNARSFIDAYKRRPDMNPTVLELAKLDFNMVQAQFQQELKEASRWWNSTGLVHELPFVRDRIVECYYWTTGVVERREHGYERIMLTKINALVTTIDDVFDIYGTLEELQLFTTAIQRWDIESMKQLPPYMQICYLALFNFVNEMAYDTLRDKGFNSTPYLRKAWVDLVESYLIEAKWYYMGHKPSLEEYMKNSWISIGGIPILSHLFFRLTDSIEEEDAESMHKYHDIVRASCTILRLADDMGTSLDEVERGDVPKSVQCYMNEKNASEEEAREHVRSLIDQTWKMMNKEMMTSSFSKYFVQVSANLARMAQWIYQHESDGFGMQHSLVNKMLRGLLFDRYEGTGENLYFQGSGGGGSDYKDDDDKGTG

SEQ ID NO: 3 - protein, 595 aa, Artificial Sequence
  Description of Artificial Sequence: Synthetic polypeptide
MVPRRMGDFHSNLWDDDVIQSLPTAYEEKSYLERAEKLIGEVENMFNSMSLEDGELMSPLNDLIQRLWIVDSLGRLGIHRHFKDEIKSALDYVYSYWGENGIGCGRESAVTDLNSTALGFRTLRLHGYPVSSDVFKAFKGQNGQFSCSENIQTDEEIRGVLNLFRASLIAFPGEKIMDEAEIFSTKYLKEALQKIPVSSLSREIGDVLEYGWHTYLPRLEARNYIHVFGQDTENTKSYVKSKKLLELAKLEFNIFQSLQKRELESLVRWWKESGFPEMTFCRHRHVEYYTLASCIAFEPQHSGFRLGFAKTCHLITVLDDMYDTFGTVDELELFTATMKRWDPSSIDCLPEYMKGVYIAVYDTVNEMAREAEEAQGRDTLTYAREAWEAYIDSYMQEARWIATGYLPSFDEYYENGKVSCGHRISALQPILTMDIPFPDHILKEVDFPSKLNDLACAILRLRGDTRCYKADRARGEEASSISCYMKDNPGVSEEDALDHINAMISDVIKGLNWELLKPDINVPISAKKHAFDIARAFHYGYKYRDGYSVANVETKSLVTRTLLESVPLGTGENLYFQGSGGGGSDYKDDDDKGTG

SEQ ID NO: 4 - protein, 590 aa, Artificial Sequence
  Description of Artificial Sequence: Synthetic polypeptide
MVPRRVGNYHSNLWDDDFIQSLISTPYGAPDYRERADRLIGEVKDIMFNFKSLEDGGNDLLQRLLLVDDVERLGIDRHFKKEIKTALDYVNSYWNEKGIGCGRESVVTDLNSTALGLRTLRLHGYTVSSDVLNVFKDKNGQFSSTANIQIEGEIRGVLNLFRASLVAFPGEKVMDEAETFSTKYLREALQKIPASSILSLEIRDVLEYGWHTNLPRLEARNYMDVFGQHTKNKNAAEKLLELAKLEFNIFHSLQERELKHVSRWWKDSGSPEMTFCRHRHVEYYALASCIAFEPQHSGFRLGFTKMSHLITVLDDMYDVFGTVDELELFTATIKRWDPSAMECLPEYMKGVYMMVYHTVNEMARVAEKAQGRDTLNYARQAWEACFDSYMQEAKWIATGYLPTFEEYLENGKVSSAHRPCALQPILTLDIPFPDHILKEVDFPSKLNDLICIILRLRGDTRCYKADRARGEEASSISCYMKDNPGLTEEDALNHINFMIRDAIRELNWELLKPDNSVPITSKKHAFDISRVWHHGYRYRDGYSFANVETKSLVMRTVIEPVPLGTGENLYFQGSGGGGSDYKDDDDKGTG

SEQ ID NO: 5 - protein, 569 aa, Artificial Sequence
  Description of Artificial Sequence: Synthetic polypeptide
MVPRRSGDYQPSLWDFNYIQSLNTPYKEQRHFNRQAELIMQVRMLLKVKMEAIQQLELIDDLQYLGLSYFFQDEIKQILSSIHNEPRYFHNNDLYFTALGFRILRQHGFNVSEDVFDCFKIEKCSDFNANLAQDTKGMLQLYEASFLLREGEDTLELARRFSTRSLREKFDEGGDEIDEDLSSWIRHSLDLPLHWRVQGLEARWFLDAYARRPDMNPLIFKLAKLNFNIVQATYQEELKDISRWWNSSCLAEKLPFVRDRIVECFFWAIAAFEPHQYSYQRKMAAVIITFITIIDDVYDVYGTIEELELLTDMIRRWDNKSISQLPYYMQVCYLALYNFVSERAYDILKDQHFNSIPYLQRSWVSLVEGYLKEAYWYYNGYKPSLEEYLNNAKISISAPTIISQLYFTLANSIDETAIESLYQYHNILYLSGTILRLADDLGTSQHELERGDVPKAIQCYMNDTNASEREAVEHVKFLIREAWKEMNTVTTASDCPFTDDLVAAAANLARAAQFIYLDGDGHGVQHSEIHQQMGGLLFQPYVGTGENLYFQGSGGGGSDYKDDDDKGTG

SEQ ID NO: 6 - protein, 594 aa, Artificial Sequence
  Description of Artificial Sequence: Synthetic polypeptide
MVPRRIGDYHSNIWDDDFIQSLSTPYGEPSYQERAERLIVEVKKIFNSMYLDDGRLMSSFNDLMQRLWIVDSVERLGIARHFKNEITSALDYVFRYWEENGIGCGRDSIVTDLNSTALGFRTLRLHGYTVSPEVLKAFQDQNGQFVCSPGQTEGEIRSVLNLYRASLIAFPGEKVMEEAEIFSTRYLKEALQKIPVSALSQEIKFVMEYGWHTNLPRLEARNYIDTLEKDTSAWLNKNAGKKLLELAKLEFNIFNSLQQKELQYLLRWWKESDLPKLTFARHRHVEFYTLASCIAIDPKHSAFRLGFAKMCHLVTVLDDIYDTFGTIDELELFTSAIKRWNSSEIEHLPEYMKCVYMVVFETVNELTREAEKTQGRNTLNYVRKAWEAYFDSYMEEAKWISNGYLPMFEEYHENGKVSSAYRVATLQPILTLNAWLPDYILKGIDFPSRFNDLASSFLRLRGDTRCYKADRDRGEEASCISCYMKDNPGSTEEDALNHINAMVNDIIKELNWELLRSNDNIPMLAKKHAFDITRALHHLYIYRDGFSVANKETKKLVMETLLESMLFGTGENLYFQGSGGGGSDYKDDDDKGTG

SEQ ID NO: 7 - protein, 828 aa, Artificial Sequence
  Description of Artificial Sequence: Synthetic polypeptide
MVPQSAEKNDSLSSSTLVKREFPPGFWKDDLIDSLTSSHKVAASDEKRIETLISEIKNMFRCMGYGETNPSAYDTAWVARIPAVDGSDNPHFPETVEWILQNQLKDGSWGEGFYFLAYDRILATLACIITLTLWRTGETQVQKGIEFFRTQAGKMEDEADSHRPSGFEIVFPAMLKEAKILGLDLPYDLPFLKQIIEKREAKLKRIPTDVLYALPTTLLYSLEGLQEIVDWQKIMKLQSKDGSFLSSPASTAAVFMRTGNKKCLDFLNFVLKKFGNHVPCHYPLDLFERLWAVDTVERLGIDRHFKEEIKEALDYVYSHWDERGIGWARENPVPDIDDTAMGLRILRLHGYNVSSDVLKTFRDENGEFFCFLGQTQRGVTDMLNVNRCSHVSFPGETIMEEAKLCTERYLRNALENVDAFDKWAFKKNIRGEVEYALKYPWHKSMPRLEARSYIENYGPDDVWLGKTVYMMPYISNEKYLELAKLDFNKVQSIHQTELQDLRRWWKSSGFTDLNFTRERVTEIYFSPASFIFEPEFSKCREVYTKTSNFTVILDDLYDAHGSLDDLKLFTESVKRWDLSLVDQMPQQMKICFVGFYNTFNDIAKEGRERQGRDVLGYIQNVWKVQLEAYTKEAEWSEAKYVPSFNEYIENASVSIALGTVVLISALFTGEVLTDEVLSKIDRESRFLQLMGLTGRLVNDTKTYQAERGQGEVASAIQCYMKDHPKISEEEALQHVYSVMENALEELNREFVNNKIPDIYKRLVFETARIMQLFYMQGDGLTLSHDMEIKEHVKNCLFQPVAGTGENLYFQGSGGGGSDYKDDDDKGTG

SEQ ID NO: 8 - protein, 831 aa, Artificial Sequence
  Description of Artificial Sequence: Synthetic polypeptide
MVPSSSTGTSKVVSETSSTIVDDIPRLSANYHGDLWHHNVIQTLETPFRESSTYQERADELVVKIKDMFNALGDGDISPSAYDTAWVARVATISSDGSEKPRFPQALNWVFNNQLQDGSWGIESHFSLCDRLLNTTNSVIALSVWKTGHSQVQQGAEFIAENLRLLNEEDELSPDFQIIFPALLQKAKALGINLPYDLPFIKYLSTTREARLTDVSAAADNIPANMLNALEGLEEVIDWNKIMRFQSKDGSFLSSPASTACVLMNTGDEKCFTFLNNLLDKFGGCVPCMYSIDLLERLSLVDNIEHLGIGRHFKQEIKGALDYVYRHWSERGIGWGRDSLVPDLNTTALGLRTLRMHGYNVSSDVLNNFKDENGRFFSSAGQTHVELRSVVNLFRASDLAFPDERAMDDARKFAEPYLREALATKISTNTKLFKEIEYVVEYPWHMSIPRLEARSYIDSYDDNYVWQRKTLYRMPSLSNSKCLELAKLDFNIVQSLHQEELKLLTRWWKESGMADINFTRHRVAEVYFSSATFEPEYSATRIAFTKIGCLQVLFDDMADIFATLDELKSFTEGVKRWDTSLLHEIPECMQTCFKVWFKLMEEVNNDVVKVQGRDMLAHIRKPWELYFNCYVQEREWLEAGYIPTFEEYLKTYAISVGLGPCTLQPILLMGELVKDDVVEKVHYPSNMFELVSLSWRLTNDTKTYQAEKVRGQQASGIACYMKDNPGATEEDAIKHICRVVDRALKEASFEYFKPSNDIPMGCKSFIFNLRLCVQIFYKFIDGYGIANEEIKDYIRKVYIDPIQVGTGENLYFQGSGGGGSDYKDDDDKGTG

SEQ ID NO: 9 - protein, 396 aa, Artificial Sequence
  Description of Artificial Sequence: Synthetic polypeptide
MVPHKFTGVNAKFQQPALRNLSPVVVEREREEFVGFFPQIVRDLTEDGIGHPEVGDAVARLKEVLQYNAPGGKCNRGLTVVAAYRELSGPGQKDAESLRCALAVGWCIELFQAFFLVADDIMDQSLTRRGQLCWYKKEGVGLDAINDSFLLESSVYRVLKKYCRQRPYYVHLLELFLQTAYQTELGQMLDLITAPVSKVDLSHFSEERYKAIVKYKTAFYSFYLPVAAAMYMVGIDSKEEHENAKAILLEMGEYFQIQDDYLDCFGDPALTGKVGTDIQDNKCSWLVVQCLQRVTPEQRQLLEDNYGRKEPEKVAKVKELYEAVGMRAAFQQYEESSYRRLQELIEKHSNRLPKEIFLGLAQKIYKRQKGTGENLYFQGSGGGGSDYKDDDDKGTG

SEQ ID NO: 10 - protein, 575 aa, Artificial Sequence
  Description of Artificial Sequence: Synthetic polypeptide
MVPSLTEEKPIRPIANFPPSIWGDQFLIYEKQVEQGVEQIVNDLKKEVRQLLKEALDIPMKHANLLKLIDEIQRLGIPYHFEREIDHALQCIYETYGDNWNGDRSSLWFRLMRKQGYYVTCDVFNNYKDKNGAFKQSLANDVEGLLELYEATSMRVPGEIILEDALGFTRSRLSIMTKDAFSTNPALFTEIQRALKQPLWKRLPRIEAAQYIPFYQQQDSHNKTLLKLAKLEFNLLQSLHKEELSHVCKWWKAFDIKKNAPCLRDRIVECYFWGLGSGYEPQYSRARVFFTKAVAVITLIDDTYDAYGTYEELKIFTEAVERWSITCLDTLPEYMKPIYKLFMDTYTEMEEFLAKEGRTDLFNCGKEFVKEFVRNLMVEAKWANEGHIPTTEEHDPVVIITGGANLLTTTCYLGMSDIFTKESVEWAVSAPPLFRYSGILGRRLNDLMTHKAEQERKHSSSSLESYMKEYNVNEEYAQTLIYKEVEDVWKDINREYLTTKNIPRPLLMAVIYLCQFLEVQYAGKDNFTRMGDEYKHLIKSLLVYPMSIGTGENLYFQGSGGGGSDYKDDDDKGTG

SEQ ID NO: 11 - skipped

SEQ ID NO: 12 - protein, 846 aa, Artificial Sequence
  Description of Artificial Sequence: Synthetic polypeptide
MVPAGVSAVSKVSSLVCDLSSTSGLIRRTANPHPNVWGYDLVHSLKSPYIDSSYRERAEVLVSEIKAMLNPAITGDGESMITPSAYDTAWVARVPAIDGSARPQFPQTVDWILKNQLKDGSWGIQSHFLLSDRLLATLSCVLVLLKWNVGDLQVEQGIEFIKSNLELVKDETDQDSLVTDFEIIFPSLLREAQSLRLGLPYDLPYIHLLQTKRQERLAKLSREEIYAVPSPLLYSLEGIQDIVEWERIMEVQSQDGSFLSSPASTACVFMHTGDAKCLEFLNSVMIKFGNFVPCLYPVDLLERLLIVDNIVRLGIYRHFEKEIKEALDYVYRHWNERGIGWGRLNPIADLETTALGFRLLRLHRYNVSPAIFDNFKDANGKFICSTGQFNKDVASMLNLYRASQLAFPGENILDEAKSFATKYLREALEKSETSSAWNNKQNLSQEIKYALKTSWHASVPRVEAKRYCQVYRPDYARIAKCVYKLPYVNNEKFLELGKLDFNIIQSIHQEEMKNVTSWFRDSGLPLFTFARERPLEFYFLVAAGTYEPQYAKCRFLFTKVACLQTVLDDMYDTYGTLDELKLFTEAVRRWDLSFTENLPDYMKLCYQIYYDIVHEVAWEAEKEQGRELVSFFRKGWEDYLLGYYEEAEWLAAEYVPTLDEYIKNGITSIGQRILLLSGVLIMDGQLLSQEALEKVDYPGRRVLTELNSLISRLADDTKTYKAEKARGELASSIECYMKDHPECTEEEALDHIYSILEPAVKELTREFLKPDDVPFACKKMLFEETRVTMVIFKDGDGFGVSKLEVKDHIKECLIEPLPLGTGENLYFQGSGGGGSDYKDDDDKGTG

SEQ ID NO: 13 - protein, 310 aa, Artificial Sequence
  Description of Artificial Sequence: Synthetic polypeptide
MVPTMMNMNFKYCHKIMKKHSKSFSYAFDLLPEDQRKAVWAIYAVCRKIDDSIDVYGDIQFLNQIKEDIQSIEKYPYEHHHFQSDRRIMMALQHVAQHKNIAFQSFYNLIDTVYKDQHFTMFETDAELFGYCYGVAGTVGEVLTPILSDHETHQTYDVARRLGESLQLINILRDVGEDFDNERIYFSKQRLKQYEVDIAEVYQNGVNNHYIDLWEYYAAIAEKDFQDVMDQIKVFSIEAQPIIELAARIYIEILDEVRQANYTLHERVFVDKRKKAKLFHENKGTGENLYFQGSGGGGSDYKDDDDKGTG

SEQ ID NO: 14 - protein, 531 aa, Artificial Sequence
  Description of Artificial Sequence: Synthetic polypeptide
MVPKIAVIGAGVTGLAAAARIASQGHEVTIFEKNNNVGGRMNQLKKDGFTFDMGPTIVMMPDVYKDVFTACGKNYEDYIELRQLRYIYDVYFDHDDRITVPTDLAELQQMLESIEPGSTHGFMSFLTDVYKKYEIARRYFLERTYRKPSDFYNMTSLVQGAKLKTLNHADQLIEHYIDNEKIQKLLAFQTLYIGIDPKRGPSLYSIIPMIEMMFGVHFIKGGMYGMAQGLAQLNKDLGVNIELNAEIEQIIIDPKFKRADAIKVNGDIRKFDKILCTADFPSVAESLMPDFAPIKKYPPHKIADLDYSCSAFLMYIGIDIDVTDQVRLHNVIFSDDFRGNIEEIFEGRLSYDPSIYVYVPAVADKSLAPEGKTGIYVLMPTPELKTGSGIDWSDEALTQQIKEIIYRKLATIEVFEDIKSHIVSETIFTPNDFEQTYHAKFGSAFGLMPTLAQSNYYRPQNVSRDYKDLYFAGASTHPGAGVPIVLTSAKITVDEMIKDIERGVGTGENLYFQGSGGGGSDYKDDDDKGTG

SEQ ID NO: 15 - protein, 325 aa, Artificial Sequence
  Description of Artificial Sequence: Synthetic polypeptide
MVPAFDFDGYMLRKAKSVNKALEAAVQMKEPLKIHESMRYSLLAGGKRVRPMLCIAACELVGGDESTAMPAACAVEMIHTMSLMHDDLPCMDNDDLRRGKPTNHMAFGESVAVLAGDALLSFAFEHVAAATKGAPPERIVRVLGELAVSIGSEGLVAGQVVDVCSEGMAEVGLDHLEFIHHHKTAALLQGSVVLGAILGGGKEEEVAKLRKFANCIGLLFQVVDDILDVTKSSKELGKTAGKDLVADKTTYPKLIGVEKSKEFADRLNREAQEQLLHFHPHRAAPLIALANYIAYRDNGTGENLYFQGSGGGGSDYKDDDDKGTG

SEQ ID NO: 16 - protein, 296 aa, Artificial Sequence
  Description of Artificial Sequence: Synthetic polypeptide
MVPSQPYWAAIEADIERYLKKSITIRPPETVFGPMHHLTFAAPATAASTLCLAACELVGGDRSQAMAAAAAIHLVHAAAYVHEHLPLTDGSRPVSKPAIQHKYGPNVELLTGDGIVPFGFELLAGSVDPARTDDPDRILRVIIEISRAGGPEGMISGLHREEEIVDGNTSLDFIEYVCKKKYGEMHACGAACGAILGGAAEEEIQKLRNFGLYQGTLRGMMEMKNSHQLIDENIIGKLKELALEELGGFHGKNAELMSSLVAEPSLYAAGTGENLYFQGSGGGGSDYKDDDDKGTG

SEQ ID NO: 17 - protein, 359 aa, Artificial Sequence
  Description of Artificial Sequence: Synthetic polypeptide
MVPLLSNKLREMVLAEVPKLASAAEYFFKRGVQGKQFRSTILLLMATALDVRVPEALIGESTDIVTSELRVRQRGIAEITEMIHVASLLHDDVLDDADTRRGVGSLNVVMGNKMSVLAGDFLLSRACGALAALKNTEVVALLATAVEHLVTGETMEITSSTEQRYSMDYYMQKTYYKTASLISNSCKAVAVLTGQTAEVAVLAFEYGRNLGLAFQLIDDILDFTGTSASLGKGSLSDIRHGVITAPILFAMEEFPQLREVVDQVEKDPRNVDIALEYLGKSKGIQRARELAMEHANLAAAAIGSLPETDNEDVKRSRRALIDLTHRVITRNKGTGENLYFQGSGGGGSDYKDDDDKGTG

SEQ ID NO: 18 - protein, 363 aa, Artificial Sequence
  Description of Artificial Sequence: Synthetic polypeptide
MVPVVSERLRHSVTTGIPALKTAAEYFFRRGIEGKRLRPTLALLMSSALSPAAPSPEYLQVDTRPAAEHPHEMRRRQQRLAEIAELIHVASLLHDDVIDDAQTRRGVLSLNTSVGNKTAILAGDFLLARASVTLASLRNSEIVELMSQVLEHLVSGEIMQMTATSEQLLDLEHYLAKTYCKTASLMANSSRSVAVLAGAAPEVCDMAWSYGRHLGIAFQVVDDLLDLTGSSSVLGKPALNDMRSGLATAPVLFAAQEEPALQALILRRFKHDGDVTKAMSLIERTQGLRRAEELAAQHAKAAADMIRCLPTAQSDHAEIAREALIQITHRVLTRKKGTGENLYFQGSGGGGSDYKDDDDKGTG

SEQ ID NO: 19 - protein, 328 aa, Artificial Sequence
  Description of Artificial Sequence: Synthetic polypeptide
MVPDFPQQLEACVKQANQALSRFIAPLPFQNTPVVETMQYGALLGGKRLRPFLVYATGHMFGVSTNTLDAPAAAVECIHAYSLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNKGTGENLYFQGSGGGGSDYKDDDDKGTG

SEQ ID NO: 20 - protein, 413 aa, Artificial Sequence
  Description of Artificial Sequence: Synthetic polypeptide
MVPSVSCCCRNLGKTIKKAIPSHHLHLRSLGGSLYRRRIQSSSMETDLKSTFLNVYSVLKSDLLHDPSFEFTNESRLWVDRMLDYNVRGGKLNRGLSVVDSFKLLKQGNDLTEQEVFLSCALGWCIEWLQAYFLVLDDIMDNSVTRRGQPCWFRVPQVGMVAINDGILLRNHIHRILKKHFRDKPYYVDLVDLFNEVELQTACGQMIDLITTFEGEKDLAKYSLSIHRRIVQYKTAYYSFYLPVACALLMAGENLENHIDVKNVLVDMGIYFQVQDDYLDCFADPETLGKIGTDIEDFKCSWLVVKALERCSEEQTKILYENYGKPDPSNVAKVKDLYKELDLEGVFMEYESKSYEKLTGAIEGHQSKAIQAVLKSFLAKIYKRQKGTGENLYFQGSGGGGSDYKDDDDKGTG

SEQ ID NO: 21 - protein, 371 aa, Artificial Sequence
  Description of Artificial Sequence: Synthetic polypeptide
MVPADLKSTFLDVYSVLKSDLLQDPSFEFTHESRQWLERMLDYNVRGGKLNRGLSVVDSYKLLKQGQDLTEKETFLSCALGWCIEWLQAYFLVLDDIMDNSVTRRGQPCWFRKPKVGMIAINDGILLRNHIHRILKKHFREMPYYVDLVDLFNEVEFQTACGQMIDLITTFDGEKDLSKYSLQIHRRIVEYKTAYYSFYLPVACALLMAGENLENHTDVKTVLVDMGIYFQVQDDYLDCFADPETLGKIGTDIEDFKCSWLVVKALERCSEEQTKILYENYGKAEPSNVAKVKALYKELDLEGAFMEYEKESYEKLTKLIEAHQSKAIQAVLKSFLAKIYKRQKGTGENLYFQGSGGGGSDYKDDDDKGTG

SEQ ID NO: 22 - protein, 389 aa, Artificial Sequence
  Description of Artificial Sequence: Synthetic polypeptide
MVPSGEPTPKKMKATYVHDRENFTKVYETLRDELLNDDCLSPAGSPQAQAAQEWFKEVNDYNVPGGKLNRGMAVYDVLASVKGPDGLSEDEVFKANALGWCIEWLQAFFLVADDIMDGSITRRGQPCWYKQPKVGMIACNDYILLECCIYSILKRHFRGHAAYAQLMDLFHETTFQTSHGQLLDLTTAPIGSVDLSKYTEDNYLRIVTYKTAYYSFYLPVACGMVLAGITDPAAFDLAKNICVEMGQYFQIQDDYLDCYGDPEVIGKIGTDIEDNKCSWLVCTALKIATEEQKEVIKANYGHKEAESVAAIKALYVELGIEQRFKDYEAASYAKLEGTISEQTLLPKAVFTSLLAKIYKRKKGTGENLYFQGSGGGGSDYKDDDDKGTG

SEQ ID NO: 23 - protein, 211 aa, Artificial Sequence
  Description of Artificial Sequence: Synthetic polypeptide
MVPQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLKGTGENLYFQGSGGGGSDYKDDDDKGTG

SEQ ID NO: 24 - protein, 322 aa, Artificial Sequence
  Description of Artificial Sequence: Synthetic polypeptide
MVPLRSLLRGLTHIPRVNSAQQPSCAHARLQFKLRSMQLLAENRTDHMRGASTWAGGQSQDELMLKDECILVDADDNITGHASKLECHKFLPHQPAGLLHRAFSVFLFDDQGRLLLQQRARSKITFPSVWANTCCSHPLHGQTPDEVDQQSQVADGTVPGAKAAAIRKLEHELGIPAHQLPASAFRFLTRLHYCAADVQPAATQSALWGEHEMDYILFIRANVTLAPNPDEVDEVRYVTQEELRQMMQPDNGLQWSPWFRIIAARFLERWWADLDAALNTDKHEDWGTVHHINEAGTGENLYFQGSGGGGSDYKDDDDKGTG

SEQ ID NO: 25 - protein, 574 aa, Artificial Sequence
  Description of Artificial Sequence: Synthetic polypeptide
MVPRRSGNYNPTAWDFNYIQSLDNQYKKERYSTRHAELTVQVKKLLEEEMEAVQKLELIEDLKNLGISYPFKDNIQQILNQIYNEHKCCHNSEVEEKDLYFTALRFRLLRQQGFEVSQEVFDHFKNEKGTDFKPNLADDTKGLLQLYEASFLLREAEDTLELARQFSTKLLQKKVDENGDDKIEDNLLLWIRRSLELPLHWRVQRLEARGFLDAYVRRPDMNPIVFELAKLDFNITQATQQEELKDLSRWWNSTGLAEKLPFARDRVVESYFWAMGTFEPHQYGYQRELVAKIIALATVVDDVYDVYGTLEELELFTDAIRRWDRESIDQLPYYMQLCFLTVNNFVFELAHDVLKDKSFNCLPHLQRSWLDLAEAYLVEAKWYHSRYTPSLEEYLNIARVSVTCPTIVSQMYFALPIPIEKPVIEIMYKYHDILYLSGMLLRLPDDLGTASFELKRGDVQKAVQCYMKERNVPENEAREHVKFLIREASKQINTAMATDCPFTEDFAVAAANLGRVANFVYVDGDGFGVQHSKIYEQIGTLMFEPYPGTGENLYFQGSGGGGSDYKDDDDKGTG

SEQ ID NO: 26 - protein, 573 aa, Artificial Sequence
  Description of Artificial Sequence: Synthetic polypeptide
MVPRRSGNYKPTMWDFQFIQSVNNLYAGDKYMERFDEVKKEMKKNLMMMVEGLIEELDVKLELIDNLERLGVSYHFKNEIMQILKSVHQQITCRDNSLYSTALKFRLLRQHGFHISQDIFNDFKDMNGNVKQSICNDTKGLLELYEASFLSTECETTLKNFTEAHLKNYVYINHSCGDQYNNIMMELVVHALELPRHWMMPRLETRWYISIYERMPNANPLLLELAKLDFNIVQATHQQDLKSLSRWWKNMCLAEKLSFSRNRLVENLFWAVGTNFEPQHSYFRRLITKIIVFVGIIDDIYDVYGKLDELELFTLAVQRWDTKAMEDLPYYMQVCYLALINTTNDVAYEVLRKHNINVLPYLTKSWTDLCKSYLQEARWYYNGYKPSLEEYMDNGWISIAVPMVLAHALFLVTDPITKEALESLTNYPDIIRCSATIFRLNDDLGTSSDELKRGDVPKSIQCYMNEKGVSEEEAREHIRFLIKETWKFMNTAHHKEKSLFCETFVEIAKNIATTAHCMYLKGDSHGIQNTDVKNSISNILFHPIIIGTGENLYFQGSGGGGSDYKDDDDKGTG

SEQ ID NO: 27 - protein, 578 aa, Artificial Sequence
  Description of Artificial Sequence: Synthetic polypeptide
MVPRRSGNYEPSAWDFNYLQSLNNYHHKEERYLRRQADLIEKVKMILKEEKMEALQQLELIDDLRNLGLSYCFDDQINHILTTIYNQHSCFHYHEAATSEEANLYFTALGFRLLREHGFKVSQEVFDRFKNEKGTDFRPDLVDDTQGLLQLYEASFLLREGEDTLEFARQFATKFLQKKVEEKMIEEENLLSWTLHSLELPLHWRIQRLEAKWFLDAYASRPDMNPIIFELAKLEFNIAQALQQEELKDLSRWWNDTGIAEKLPFARDRIVESHYWAIGTLEPYQYRYQRSLIAKIIALTTVVDDVYDVYGTLDELQLFTDAIRRWDIESINQLPSYMQLCYLAIYNFVSELAYDIFRDKGFNSLPYLHKSWLDLVEAYFQEAKWYHSGYTPSLEQYLNIAQISVASPAILSQIYFTMAGSIDKPVIESMYKYRHILNLSGILLRLPDDLGTASDELGRGDLAKAMQCYMKERNVSEEEARDHVRFLNREVSKQMNPARAADDCPFTDDFVVAAANLGRVADFMYVEGDGLGLQYPAIHQHMAELLFHPYAGTGENLYFQGSGGGGSDYKDDDDKGTG

SEQ ID NO: 28 - protein, 578 aa, Artificial Sequence
  Description of Artificial Sequence: Synthetic polypeptide
MVPRRSGNYQPSAWDFNYIQSLNNNHSKEERHLERKAKLIEEVKMLLEQEMAAVQQLELIEDLKNLGLSYLFQDEIKIILNSIYNHHKCFHNNHEQCIHVNSDLYFVALGFRLFRQHGFKVSQEVFDCFKNEEGSDFSANLADDTKGLLQLYEASYLVTEDEDTLEMARQFSTKILQKKVEEKMIEKENLLSWTLHSLELPLHWRIQRLEAKWFLDAYASRPDMNPIIFELAKLEFNIAQALQQEELKDLSRWWNDTGIAEKLPFARDRIVESHYWAIGTLEPYQYRYQRSLIAKIIALTTVVDDVYDVYGTLDELQLFTDAIRRWDIESINQLPSYMQLCYLAIYNFVSELAYDIFRDKGFNSLPYLHKSWLDLVEAYFVEAKWFHDGYTPTLEEYLNNSKITIICPAIVSEIYFAFANSIDKTEVESIYKYHDILYLSGMLARLPDDLGTSSFEMKRGDVAKAIQCYMKEHNASEEEAREHIRFLMREAWKHMNTAAAADDCPFESDLVVGAASLGRVANFVYVEGDGFGVQHSKIHQQMAELLFYPYQGTGENLYFQGSGGGGSDYKDDDDKGTG

SEQ ID NO: 29 - protein, 568 aa, Artificial Sequence
  Description of Artificial Sequence: Synthetic polypeptide
MVPRRSANYQASIWDDNFIQSLASPYAGEKYAEKAEKLKTEVKTMIDQTRDELKQLELIDNLQRLGICHHFQDLTKKILQKIYGEERNGDHQHYKEKGLHFTALRFRILRQDGYHVPQDVFSSFMNKAGDFEESLSKDTKGLVSLYEASYLSMEGETILDMAKDFSSHHLHKMVEDATDKRVANQIIHSLEMPLHRRVQKLEAIWFIQFYECGSDANPTLVELAKLDFNMVQATYQEELKRLSRWYEETGLQEKLSFARHRLAEAFLWSMGIIPEGHFGYGRMHLMKIGAYITLLDDIYDVYGTLEELQVLTEIIERWDINLLDQLPEYMQIFFLYMFNSTNELAYEILRDQGINVISNLKGLWVELSQCYFKEATWFHNGYTPTTEEYLNVACISASGPVILFSGYFTTTNPINKHELQSLERHAHSLSMILRLADDLGTSSDEMKRGDVPKAIQCFMNDTGCCEEEARQHVKRLIDAEWKKMNKDILMEKPFKNFCPTAMNLGRISMSFYEHGDGYGGPHSDTKKKMVSLFVQPMNITIGTGENLYFQGSGGGGSDYKDDDDKGTG

SEQ ID NO: 30 - protein, 571 aa, Artificial Sequence
  Description of Artificial Sequence: Synthetic polypeptide
MVPRRSANYQPSIWNHDYIESLRIEYVGETCTRQINVLKEQVRMMLHKVVNPLEQLELIEILQRLGLSYHFEEEIKRILDGVYNNDHGGDTWKAENLYATALKFRLLRQHGYSVSQEVFNSFKDERGSFKACLCEDTKGMLSLYEASFFLIEGENILEEARDFSTKHLEEYVKQNKEKNLATLVNHSLEFPLHWRMPRLEARWFINIYRHNQDVNPILLEFAELDFNIVQAAHQADLKQVSTWWKSTGLVENLSFARDRPVENFFWTVGLIFQPQFGYCRRMFTKVFALITTIDDVYDVYGTLDELELFTDVVERWDINAMDQLPDYMKICFLTLHNSVNEMALDTMKEQRFHIIKYLKKAWVDLCRYYLVEAKWYSNKYRPSLQEYIENAWISIGAPTILVHAYFFVTNPITKEALDCLEEYPNIIRWSSIIARLADDLGTSTDELKRGDVPKAIQCYMNETGASEEGAREYIKYLISATWKKMNKDRAASSPFSHIFIEIALNLARMAQCLYQHGDGHGLGNRETKDRILSLLIQPIPLNKDGTGENLYFQGSGGGGSDYKDDDDKGTG

SEQ ID NO: 31 - protein, 599 aa, Artificial Sequence
  Description of Artificial Sequence: Synthetic polypeptide
MVPRRIGDYHSNLWNDDFIQSLTTPYGAPSYIERADRLISEVKEMFNRMCMEDGELMSPLNDLIQRLWTVDSVERLGIDRHFKNEIKASLDYVYSYWNEKGIGCGRQSVVTDLNSTALGLRILRQHGYTVSSEVLKVFEEENGQFACSPSQTEGEIRSFLNLYRASLIAFPGEKVMEEAQIFSSRYLKEAVQKIPVSGLSREIGDVLEYGWHTNLPRWEARNYMDVFGQDTNTSFNKNKMQYMNTEKILQLVKLEFNIFHSLQQRELQCLLRWWKESGLPQLTFARHRHVEFYTLASCIACEPKHSAFRLGFAKMCHLVTVLDDVYDTFGKMDELELFTAAVKRWDLSETERLPEYMKGLYVVVFETVNELAQEAEKTQGRNTLNYVRKAWEAYFDSYMKEAEWISTGYLPTFEEYCENGKVSSAYRVAALQPILTLDVQLPDDILKGIDFPSRFNDLASSFLRLRGDTRCYEADRARGEEASCISCYMKDNPGSTEEDALNHINAMINDIIRELNWEFLKPDSNIPMPARKHAFDITRALHHLYIYRDGFSVANKETKNLVEKTLLESMLFGTGENLYFQGSGGGGSDYKDDDDKGTG

SEQ ID NO: 32 - protein, 576 aa, Artificial Sequence
  Description of Artificial Sequence: Synthetic polypeptide
MVPRRSANYQPSRWDHHHLLSVENKFAKDKRVRERDLLKEKVRKMLNDEQKTYLDQLEFIDDLQKLGVSYHFEAEIDNILTSSYKKDRTNIQESDLHATALEFRLFRQHGFNVSEDVFDVFMENCGKFDRDDIYGLISLYEASYLSTKLDKNLQIFIRPFATQQLRDFVDTHSNEDFGSCDMVEIVVQALDMPYYWQMRRLSTRWYIDVYGKRQNYKNLVVVEFAKIDFNIVQAIHQEELKNVSSWWMETGLGKQLYFARDRIVENYFWTIGQIQEPQYGYVRQTMTKINALLTTIDDIYDIYGTLEELQLFTVAFENWDINRLDELPEYMRLCFLVIYNEVNSIACEILRTKNINVIPFLKKSWTDVSKAYLVEAKWYKSGHKPNLEEYMQNARISISSPTIFVHFYCVFSDQLSIQVLETLSQHQQNVVRCSSSVFRLANDLVTSPDELARGDVCKSIQCYMSETGASEDKARSHVRQMINDLWDEMNYEKMAHSSSILHHDFMETVINLARMSQCMYQYGDGHGSPEKAKIVDRVMSLLFNPIPLDGTGENLYFQGSGGGGSDYKDDDDKGTG

SEQ ID NO: 33 - protein, 570 aa, Artificial Sequence
  Description of Artificial Sequence: Synthetic polypeptide
MVPRRSANYQPSLWQHEYLLSLGNTYVKEDNVERVTLLKQEVSKMLNETEGLLEQLELIDTLQRLGVSYHFEQEIKKTLTNVHVKNVRAHKNRIDRNRWGDLYATALEFRLLRQHGFSIAQDVFDGNIGVDLDDKDIKGILSLYEASYLSTRIDTKLKESIYYTTKRLRKFVEVNKNETKSYTLRRMVIHALEMPYHRRVGRLEARWYIEVYGERHDMNPILLELAKLDFNFVQAIHQDELKSLSSWWSKTGLTKHLDFVRDRITEGYFSSVGVMYEPEFAYHRQMLTKVFMLITTIDDIYDIYGTLEELQLFTTIVEKWDVNRLEELPNYMKLCFLCLVNEINQIGYFVLRDKGFNVIPYLKESWADMCTTFLKEAKWYKSGYKPNFEEYMQNGWISSSVPTILLHLFCLLSDQTLDILGSYNHSVVRSSATILRLANDLATSSEELARGDTMKSVQCHMHETGASEAESRAYIQGIIGVAWDDLNMEKKSCRLHQGFLEAAANLGRVAQCVYQYGDGHGCPDKAKTVNHVRSLLVHPLPLNGTGENLYFQGSGGGGSDYKDDDDKGTG

SEQ ID NO: 34 - protein, 583 aa, Artificial Sequence
  Description of Artificial Sequence: Synthetic polypeptide
MVPASPPAHRSSKAADEELPKASSTFHPSLWGSFFLTYQPPTAPQRANMKERAEVLRERVRKVLKGSTTDQLPETVNLILTLQRLGLGYYYENEIDKLLHQIYSNSDYNVKDLNLVSQRFYLLRKNGYDVPSDVFLSFKTEEGGFACAAADTRSLLSLYNAAYLRKHGEEVLDEAISSTRLRLQDLLGRLLPESPFAKEVSSSLRTPLFRRVGILEARNYIPIYETEATRNEAVLELAKLNFNLQQLDFCEELKHCSAWWNEMIAKSKLTFVRDRIVEEYFWMNGACYDPPYSLSRIILTKITGLITIIDDMFDTHGTTEDCMKFAEAFGRWDESAIHLLPEYMKDFYILMLETFQSFEDALGPEKSYRVLYLKQAMERLVELYSKEIKWRDDDYVPTMSEHLQVSAETIATIALTCSAYAGMGDMSIRKETFEWALSFPQFIRTFGSFVRLSNDVVSTKREQTKDHSPSTVHCYMKEHGTTMDDACEKIKELIEDSWKDMLEQSLALKGLPKVVPQLVFDFSRTTDNMYRDRDALTSSEALKEMIQLLFVEPIPEGTGENLYFQGSGGGGSDYKDDDDKGTG

SEQ ID NO: 35 - protein, 586 aa, Artificial Sequence
  Description of Artificial Sequence: Synthetic polypeptide
MVPEALGNFDYESYTNFTKLPSSQWGDQFLKFSIADSDFDVLEREIEVLKPKVRENIFVSSSTDKDAMKKTILSIHFLDSLGLSYHFEKEIEESLKHAFEKIEDLIADENKLHTISTIFRVFRTYGYYMSSDVFKIFKGDDGKFKESLIEDVKGMLSFYEAVHFGTTTDHILDEALSFTLNHLESLATGRRASPPHISKLIQNALHIPQHRNIQALVAREYISFYEHEEDHDETLLKLAKLNFKFLQLHYFQELKTITMWWTKLDHTSNLPPNFRERTVETWFAALMMYFEPQFSLGRIMSAKLYLVITFLDDACDTYGSISEVESLADCLERWDPDYMENLQGHMKTAFKFVMYLFKEYEEILRSQGRSFVLEKMIEEFKIIARKNLELVKWARGGHVPSFDEYIESGGAEIGTYATIACSIMGLGEIGKKEAFEWLISRPKLVRILGAKTRLMDDIADFEEDMEKGYTANALNYYMNEHGVTKEEASRELEKMNGDMNKIVNEECLKITTMPRRILMQSVNYARSLDVLYTADDVYNHREGKLKEYMRLLLVDPILLGTGENLYFQGSGGGGSDYKDDDDKGTG

SEQ ID NO: 36 - protein, 553 aa, Artificial Sequence
  Description of Artificial Sequence: Synthetic polypeptide
MVPESQTTFKYESLAFTKLSHCQWTDYFLSVPIDESELDVITREIDILKPEVMELLSSQGDDETSKRKVLLIQLLLSLGLAFHFENEIKNILEHAFRKIDDITGDEKDLSTISIMFRVFRTYGHNLPSSVFKRFTGDDGKFQQSLTEDAKGILSLYEAAHLGTTTDYILDEALKFTSSHLKSLLAGGTCRPHILRLIRNTLYLPQRWNMEAVIAREYISFYEQEEDHDKMLLRLAKLNFKLLQLHYIKELKSFIKWWMELGLTSKWPSQFRERIVEAWLAGLMMYFEPQFSGGRVIAAKFNYLLTILDDACDHYFSIHELTRLVACVERWSPDGIDTLEDISRSVFKLMLDVFDDIGKGVRSEGSSYHLKEMLEELNTLVRANLDLVKWARGIQTAGKEAYEWVRSRPRLIKSLAAKGRLMDDITDFDSDMSNGFAANAINYYMKQFVVTKEEAILECQRMIVDINKTINEELLKTTSVPGRVLKQALNFGRLLELLYTKSDDIYNCSEGKLKEYIVTLLIDPIRLGTGENLYFQGSGGGGSDYKDDDDKGTG

SEQ ID NO: 37 - protein, 500 aa, Artificial Sequence
  Description of Artificial Sequence: Synthetic polypeptide
MVPESQTKFDYESLAFTKLSHSQWTDYFLSVPIDDSELDAITREIDIIKPEVRKLLSSKGDDETSKRKVLLIQSLLSLGLAFHFENEIKDILEDAFRRIDDITGDENDLSTISIMFRVFRTYGHNLPSSVFKRFTGDDGKFERSLTEDAKGILSLYEAAHLGTTTDYILDEALEFTSSHLKSLLVGGMCRPHILRLIRNTLYLPQRWNMEAVIAREYISFYEQEEDHDKMLLRLAKLNFKLLQLHYIKELKTFIKWWMELGLTSKWPSQFRERIVEAWLAGLMMYFEPQFSGGRVIAAKFNYLLTILDDACDHYFSIPELTRLVDCVERWNHDGIHTLEDISRIIFKLALDVFDDIGRGVRSKGCSYYLKEMLEELKILVRANLDLVKWARGNQLPSFEEHVEVGGIALTTYATLMYSFVGMGEAVGKEAYEWVRSRPRLIKSLAAKGRLMDDITDFEVKIINLFFDLLLFVFGTGENLYFQGSGGGGSDYKDDDDKGTG

SEQ ID NO: 38 - protein, 574 aa, Artificial Sequence
  Description of Artificial Sequence: Synthetic polypeptide
MVPAAFTANAVDMRPPVITIHPRSKDIFSQFSLDDKLQKQYAQGIEALKEEARSMLMAAKSAKVMILIDTLERLGLGYHFEKEIEEKLEAIYKKEDGDDYDLFTTALRFRLLRQHQRRVPCSVFDKFMNKEGKFEEEPLISDVEGLLSLYDAAYLQIHGEHILQEALIFTTHHLTRIEPQLDDHSPLKLKLNRALEFPFYREIPIIYAHFYISVYERDDSRDEVLLKMAKLSYNFLQNLYKKELSQLSRWWNKLELIPNLPYIRDSVAGAYLWAVALYFEPQYSDVRMAIAKLIQIAAAVDDTYDNYATIREAQLLTEALERLNVHEIDTLPDYMKIVYRFVMSWSEDFERDATIKEQMLATPYFKAEMKKLGRAYNQELKWVMERQLPSFEEYMKNSEITSGVYIMFTVISPYLNSATQKNIDWLLSQPRLASSTAIVMRCCNDLGSNQRESKGGEVMTSLDCYMKQHGASKQETISKFKLIIEDEWKNLNEEWAATTCLPKVMVEIFRNYARIAGFCYKNNGDAYTSPKIVQQCFDALFVNPLRIGTGENLYFQGSGGGGSDYKDDDDKGTG

SEQ ID NO: 39 - protein, 605 aa, Artificial Sequence
  Description of Artificial Sequence: Synthetic polypeptide
MVPEFRVHLQADNEQKIFQNQMKPEPEASYLINQRRSANYKPNIWKNDFLDQSLISKYDGDEYRKLSEKLIEEVKIYISAETMDLVAKLELIDSVRKLGLANLFEKEIKEALDSIAAIESDNLGTRDDLYGTALHFKILRQHGYKVSQDIFGRFMDEKGTLENHHFAHLKGMLELFEASNLGFEGEDILDEAKASLTLALRDSGHICYPDSNLSRDVVHSLELPSHRRVQWFDVKWQINAYEKDICRVNATLLELAKLNFNVVQAQLQKNLREASRWWANLGFADNLKFARDRLVECFSCAVGVAFEPEHSSFRICLTKVINLVLIIDDVYDIYGSEEELKHFTNAVDRWDSRETEQLPECMKMCFQVLYNTTCEIAREIEEENGWNQVLPQLTKVWADFCKALLVEAEWYNKSHIPTLEEYLRNGCISSSVSVLLVHSFFSITHEGTKEMADFLHKNEDLLYNISLIVRLNNDLGTSAAEQERGDSPSSIVCYMREVNASEETARKNIKGMIPRTWKKVNGKCFTTNQVPFLSSFMNNATNMARVAHSLYKDGDGFGDQEKGPRTHILSLLFQPLVNGTGENLYFQGSGGGGSDYKDDDDKGTG

SEQ ID NO: 40 - protein, 590 aa, Artificial Sequence
  Description of Artificial Sequence: Synthetic polypeptide
MVPSSNVSAIPNSFELIRRSAQFQASVWGDYFLSYHSLPPEKGNKVMEKQTEELKEEIKMELVSTTKDEPEKLRLIDLIQRLGVCYHFENEINNILQQLHHITITSEKNGDDNPYNMTLCFRLLRQQGYNVSSEPFDRFRGKWESSYDNNVEELLSLYEASQLRMQGEEALDEAFCFATAQLEAIVQDPTTDPMVAAEIRQALKWPMYKNLPRLKARHHIGLYSEKPWRNESLLNFAKMDFNKLQNLHQTEIAYISKWWDDYGFAEKLSFARNRIVEGYFFALGIFFEPQLLTARLIMTKVIAIGSMLDDIYDVYGTFEELKLLTLALERWDKSETKQLPNYMKMYYEALLDVFEEIEQEMSQKETETTPYCIHHMKEATKELGRVFLVEATWCKEGYTPKVEEYLDIALISFGHKLLMVTALLGMGSHMATQQIVQWITSMPNILKASAVICRLMNDIVSHKFEQERGHVASAIECYMEQNHLSEYEALIALRKQIDDLWKDMVENYCAVITEDEVPRGVLMRVLNLTRLFNVIYKDGDGYTQSHGSTKAHIKSLLVDSVPLGTGENLYFQGSGGGGSDYKDDDDKGTG

SEQ ID NO: 41 - protein, 589 aa, Artificial Sequence
  Description of Artificial Sequence: Synthetic polypeptide
MVPKDMSIPLLAAVSSSTEETVRPIADFHPTLWGNHFLKSAADVETIDAATQEQHAALKQEVRRMITTTANKLAQKLHMIDAVQRLGVAYHFEKEIEDELGKVSHDLDSDDLYVVSLRFRLFRQQGVKISCDVFDKFKDDEGKFKESLINDIRGMLSLYEAAYLAIRGEDILDEAIVFTTTHLKSVISISDHSHANSNLAEQIRHSLQIPLRKAAARLEARYFLDIYSRDDLHDETLLKFAKLDFNILQAAHQKEASIMTRWWNDLGFPKKVPYARDRIIETYIWMLLGVSYEPNLAFGRIFASKVVCMITTIDDTFDAYGTFEELTLFTEAVTRWDIGLIDTLPEYMKFIVKALLDIYREAEEELAKEGRSYGIPYAKQMMQELIILYFTEAKWLYKGYVPTFDEYKSVALRSIGLRTLAVASFVDLGDFIATKDNFECILKNAKSLKATETIGRLMDDIAGYKFEQKRGHNPSAVECYKNQHGVSEEEAVKELLLEVANSWKDINEELLNPTTVPLPMLQRLLYFARSGHFIYDDGHDRYTHSLMMKRQVALLLTEPLAIGTGENLYFQGSGGGGSDYKDDDDKGTG

SEQ ID NO: 42 - protein, 609 aa, Artificial Sequence
  Description of Artificial Sequence: Synthetic polypeptide
MVPDLAVEIAMDLAVDDVERRVGDYHSNLWDDDFIQSLSTPYGASSYRERAERLVGEVKEMFTSISIEDGELTSDLLQRLWMVDNVERLGISRHFENEIKAAIDYVYSYWSDKGIVRGRDSAVPDLNSIALGFRTLRLHGYTVSSDVFKVFQDRKGEFACSAIPTEGDIKGVLNLLRASYIAFPGEKVMEKAQTFAATYLKEALQKIQVSSLSREIEYVLEYGWLTNFPRLEARNYIDVFGEEICPYFKKPCIMVDKLLELAKLEFNLFHSLQQTELKHVSRWWKDSGFSQLTFTRHRHVEFYTLASCIAIEPKHSAFRLGFAKVCYLGIVLDDIYDTFGKMKELELFTAAIKRWDPSTTECLPEYMKGVYMAFYNCVNELALQAEKTQGRDMLNYARKAWEALFDAFLEEAKWISSGYLPTFEEYLENGKVSFGYRAATLQPILTLDIPLPLHILQQIDFPSRFNDLASSILRLRGDICGYQAERSRGEEASSISCYMKDNPGSTEEDALSHINAMISDNINELNWELLKPNSNVPISSKKHAFDILRAFYHLYKYRDGFSIAKIETKNLVMRTVLEPVPMGTGENLYFQGSGGGGSDYKDDDDKGTG

SEQ ID NO: 43 - protein, 836 aa, Artificial Sequence
  Description of Artificial Sequence: Synthetic polypeptide
MVPTSVSVESGTVSCLSSNNLIRRTANPHPNIWGYDFVHSLKSPYTHDSSYRERAETLISEIKVMLGGGELMMTPSAYDTAWVARVPSIDGSACPQFPQTVEWILKNQLKDGSWGTESHFLLSDRLLATLSCVLALLKWKVADVQVEQGIEFIKRNLQAIKDERDQDSLVTDFEIIFPSLLKEAQSLNLGLPYDLPYIRLLQTKRQERLANLSMDKIHGGTLLSSLEGIQDIVEWETIMDVQSQDGSFLSSPASTACVFMHTGDMKCLDFLNNVLTKFGSSVPCLYPVDLLERLLIVDNVERLGIDRHFEKEIKEALDYVYRHWNDRGIGWGRLSPIADLETTALGFRLLRLHRYNVSPVVLDNFKDADGEFFCSTGQFNKDVASMLSLYRASQLAFPEESILDEAKSFSTQYLREALEKSETFSSWNHRQSLSEEIKYALKTSWHASVPRVEAKRYCQVYRQDYAHLAKSVYKLPKVNNEKILELAKLDFNIIQSIHQKEMKNVTSWFRDSGLPLFTFARERPLEFYFLIAGGTYEPQYAKCRFLFTKVACLQTVLDDMYDTYGTPSELKLFTEAVRRWDLSFTENLPDYMKLCYKIYYDIVHEVAWEVEKEQGRELVSFFRKGWEDYLLGYYEEAEWLAAEYVPTLDEYIKNGITSIGQRILLLSGVLIMEGQLLSQEALEKVDYPGRRVLTELNSLISRLADDTKTYKAEKARGELASSIECYMKDHPGCQEEEALNHIYGILEPAVKELTREFLKADHVPFPCKKMLFDETRVTMVIFKDGDGFGISKLEVKDHIKECLIEPLPLGTGENLYFQGSGGGGSDYKDDDDKGTG

SEQ ID NO: 44 - protein, 576 aa, Artificial Sequence
  Description of Artificial Sequence: Synthetic polypeptide
MVPGSEVNRPLADFPANIWEDPLTSFSKSDLGTETFKEKHSTLKEAVKEAFMSSKANPIENIKFIDALCRLGVSYHFEKDIVEQLDKSFDCLDFPQMVRQEGCDLYTVGIIFQVFRQFGFKLSADVFEKFKDENGKFKGHLVTDAYGMLSLYEAAQWGTHGEDIIDEALAFSRSHLEEISSRSSPHLAIRIKNALKHPYHKGISRIETRQYISYYEEEESCDPTLLEFAKIDFNLLQILHREELACVTRWHHEMEFKSKVTYTRHRITEAYLWSLGTYFEPQYSQARVITTMALILFTALDDMYDAYGTMEELELFTDAMDEWLPVVPDEIPIPDSMKFIYNVTVEFYDKLDEELEKEGRSGCGFHLKKSLQKTANGYMQEAKWLKKDYIATFDEYKENAILSSGYYALIAMTFVRMTDVAKLDAFEWLSSHPKIRVASEIISRFTDDISSYEFEHKREHVATGIDCYMQQFGVSKERAVEVMGNIVSDAWKDLNQELMRPHVFPFPLLMRVLNLSRVIDVFYRYQDAYTNPKLLKEHIVSLLIETIPIGTGENLYFQGSGGGGSDYKDDDDKGTG

SEQ ID NO: 45 - protein, 590 aa, Artificial Sequence
  Description of Artificial Sequence: Synthetic polypeptide
MVPEAIRVFGLKLGSKLSIHSQTNAFPAFKLSRFPLTSFPGKHAHLDPLKATTHPLAFDGEENNREFKNLGPSEWGHQFLSAHVDLSEMDALEREIEALKPKVRDMLISSESSKKKILFLYLLVSLGLAYHFEDEIKESLEDGLQKIEEMMASEDDLRFKGDNGKFKECLAKDAKGILSLYEAAHMGTTTDYILDEALSFTLTYMESLAASGTCKINLSRRIRKALDQPQHKNMEIIVAMKYIQFYEEEEDCDKTLLKFAKLNFKFLQLHYLQELKILSKWYKDQDFKSKLPPYFRDRLVECHFASLTCFEPKYARARIFLSKIFTVQIFIDDTCDRYASLGEVESLADTIERWDPDDHAMDGLPDYLKSVVKFVFNTFQEFERKCKRSLRINLQVAKWVKAGHLPSFDEYLDVAGLELAISFTFAGILMGMENVCKPEAYEWLKSRDKLVRGVITKVRLLNDIFGYEDDMRRGYVTNSINCYKKQYGVTEEEAIRKLHQIVADGEKMMNEEFLKPINVPYQVPKVVILDTLRAANVSYEKDDEFTRPGEHLKNCITSIYFDLGTGENLYFQGSGGGGSDYKDDDDKGTG

SEQ ID NO: 46 - protein, 375 aa, Artificial Sequence
  Description of Artificial Sequence: Synthetic polypeptide
MVPTTTLSSNLNSQFMQVYETLKSELIHDPLFEFDDDSRQWVERMIDYTVPGGKMVRGYSVVDSYQLLKGEELTEEEAFLACALGWCTEWFQAFILLHDDMMDGSHTRRGQPCWFRLPEVGAVAINDGVLLRNHVHRILKKHFQGKAYYVHLVDLFNETEFQTISGQMIDLITTLVGEKDLSKYSLSIHRRIVQYKTAYYSFYLPVACALLMFGEDLDKHVEVKNVLVEMGTYFQVQDDYLDCFGAPEVIGKIGTDIEDFKCSWLVVKALELANEEQKKTLHENYGKKDPASVAKVKEVYHTLNLQAVFEDYEATSYKKLITSIENHPSKAVQAVLKSFLGKIYKRQKGTGENLYFQGSGGGGSDYKDDDDKGTG

SEQ ID NO: 47 - protein, 600 aa, Artificial Sequence
  Description of Artificial Sequence: Synthetic polypeptide
MVPSQPYWAAIEADIERYLKKSITIRPPETVFGPMHHLTFAAPATAASTLCLAACELVGGDRSQAMAAAAAIHLVHAAAYVHEHLPLTDGSRPVSKPAIQHKYGPNVELLTGDGIVPFGFELLAGSVDPARTDDPDRILRVIIEISRAGGPEGMISGLHREEEIVDGNTSLDFIEYVCKKKYGEMHACGAACGAILGGAAEEEIQKLRNFGLYQGTLRGMMEMKNSHQLIDENIIGKLKELALEELGGFHGKNAELMSSLVAEPSLYAASSNNLGIEGRFDFDGYMLRKAKSVNKALEAAVQMKEPLKIHESMRYSLLAGGKRVRPMLCIAACELVGGDESTAMPAACAVEMIHTMSLMHDDLPCMDNDDLRRGKPTNHMAFGESVAVLAGDALLSFAFEHVAAATKGAPPERIVRVLGELAVSIGSEGLVAGQVVDVCSEGMAEVGLDHLEFIHHHKTAALLQGSVVLGAILGGGKEEEVAKLRKFANCIGLLFQVVDDILDVTKSSKELGKTAGKDLVADKTTYPKLIGVEKSKEFADRLNREAQEQLLHFHPHRAAPLIALANYIAYRDNGTGENLYFQGSGGGGSDYKDDDDKGTG

SEQ ID NO: 48 - protein, 456 aa, Artificial Sequence
  Description of Artificial Sequence: Synthetic polypeptide
MVPVTAARATPKLSNRKLRVAVIGGGPAGGAAAETLAQGGIETILIERKMDNCKPCGGAIPLCMVGEFNLPLDIIDRRVTKMKMISPSNIAVDIGRTLKEHEYIGMVRREVLDAYLRERAEKSGATVINGLFLKMDHPENWDSPYTLHYTEYDGKTGATGTKKTMEVDAVIGADGANSRVAKSIDAGDYDYAIAFQERIRIPDEKMTYYEDLAEMYVGDDVSPDFYGWVFPKCDHVAVGTGTVTHKGDIKKFQLATRNRAKDKILGGKIIRVEAHPIPEHPRPRRLSKRVALVGDAAGYVTKCSGEGIYFAAKSGRMCAEAIVEGSQNGKKMIDEGDLRKYLEKWDKTYLPTYRVLDVLQKVFYRSNPAREAFVEMCNDEYVQKMTFDSYLYKRVAPGSPLEDIKLAVNTIGSLVRANALRREIEKLSVGTGENLYFQGSGGGGSDYKDDDDKGTG

SEQ ID NO: 49 - protein, 453 aa, Artificial Sequence
  Description of Artificial Sequence: Synthetic polypeptide
MVPVAVIGGGPSGACAAETLAKGGVETFLLERKLDNCKPCGGAIPLCMVEEFDLPMEIIDRRVTKMKMISPSNREVDVGKTLSETEWIGMCRREVFDDYLRNRAQKLGANIVNGLFMRSEQQSAEGPFTIHYNSYEDGSKMGKPATLEVDMIIGADGANSRIAKEIDAGEYDYAIAFQERIRIPDDKMKYYENLAEMYVGDDVSPDFYGWVFPKYDHVAVGTGTVVNKTAIKQYQQATRDRSKVKTEGGKIIRVEAHPIPEHPRPRRCKGRVALVGDAAGYVTKCSGEGIYFAAKSGRMAAEAIVEGSANGTKMCGEDAIRVYLDKWDRKYWTTYKVLDILQKVFYRSNPAREAFVELCEDSYVQKMTFDSYLYKTVVPGNPLDDVKLLVRTVSSILRSNALRSVNSKSVNVSFGSKANEERVMAAGTGENLYFQGSGGGGSDYKDDDDKGTG

SEQ ID NO: 50 - protein, 305 aa, Artificial Sequence
  Description of Artificial Sequence: Synthetic polypeptide
MVPAMAVPLDVVITYPSSGAAAYPVLVMYNGFQAKAPWYRGIVDHVSSWGYTVVQYTNGGLFPIVVDRVELTYLEPLLTWLETQSADAKSPLYGRADVSRLGTMGHSRGGKLAALQFAGRTDVSGCVLFDPVDGSPMTPESADYPSATKALAAAGRSAGLVGAAITGSCNPVGQNYPKFWGALAPGSWQMVLSQAGHMQFARTGNPFLDWSLDRLCGRGTMMSSDVITYSAAFTVAWFEGIFRPAQSQMGISNFKTWANTQVAARSITFDIKPMQSPQGTGENLYFQGSGGGGSDYKDDDDKGTG

SEQ ID NO: 51 - protein, 315 aa, Artificial Sequence
  Description of Artificial Sequence: Synthetic polypeptide
MVPAPPKPVRITCPTVAGTYPVVLFFHGFYLRNYFYSDVLNHIASHGYILVAPQLCKLLPPGGQVEVDDAGSVINWASENLKAHLPTSVNANGKYTSLVGHSRGGKTAFAVALGHAATLDPSITFSALIGIDPVAGTNKYIRTDPHILTYKPESFELDIPVAVVGTGLGPKWNNVMPPCAPTDLNHEEFYKECKATKAHFVAADYGHMDMLDDDLPGFVGFMAGCMCKNGQRKKSEMRSFVGGIVVAFLKYSLWGEKAEIRLIVKDPSVSPAKLDPSPELEEASGIFVGTGENLYFQGSGGGGSDYKDDDDKGTG

SEQ ID NO: 52 - protein, 301 aa, Artificial Sequence
  Description of Artificial Sequence: Synthetic polypeptide
MVPATPVEEGDYPVVMLLHGYLLYNSFYSQLMLHVSSHGFILIAPQLYSIAGPDTMDEIKSTAEIMDWLSVGLNHFLPAQVTPNLSKFALSGHSRGGKTAFAVALKKFGYSSNLKISTLIGIDPVDGTGKGKQTPPPVLAYLPNSFDLDKTPILVIGSGLGETARNPLFPPCAPPGVNHREFFRECQGPAWHFVAKDYGHLDMLDDDTKGIRGKSSYCLCKNGEERRPMRRFVGGLVVSFLKAYLEGDDRELVKIKDGCHEDVPVEIQEFEVIMGTGENLYFQGSGGGGSDYKDDDDKGTG

SEQ ID NO: 53 - protein, 534 aa, Artificial Sequence
  Description of Artificial Sequence: Synthetic polypeptide
MVPSHKKKNVIFFVTDGMGPASLSMARSFNQHVNDLPIDDILTLDEHFIGSSRTRSSDSLVTDSAAGATAFACALKSYNGAIGVDPHHRPCGTVLEAAKLAGYLTGLVVTTRITDATPASFSSHVDYRWQEDLIATHQLGEYPLGRVVDLLMGGGRSHFYPQGEKASPYGHHGARKDGRDLIDEAQSNGWQYVGDRKNFDSLLKSHGENVTLPFLGLFADNDIPFEIDRDEKEYPSLKEQVKVALGALEKASNEDKDSNGFFLMVEGSRIDHAGHQNDPASQVREVLAFDEAFQYVLEFAENSDTETVLVSTSDHETGGLVTSRQVTASYPQYVWYPQVLANATHSGEFLKRKLVDFVHEHKGASSKIENFIKHEILEKDLGIYDYTDSDLETLIHLDPRTNAIQDKLNDMVSFRAQIGWTTHGHSAVDVNIYAYANKKATWSYVLNNLQGNHENTEVGQFLENFLELNLNEVTDLIRDTKHTSDFDATEIASEVQHYDEYYHELTNGTGENLYFQGSGGGGSDYKDDDDKGTG

SEQ ID NO: 54 - protein, 396 aa, Artificial Sequence
  Description of Artificial Sequence: Synthetic polypeptide
MVPHKFTGVNAKFQQPALRNLSPVVVEREREEFVGFFPQIVRDLTEDGIGHPEVGDAVARLKEVLQYNAPGGKCNRGLTVVAAYRELSGPGQKDAESLRCALAVGWCIELFQAFFLVWDDIMDQSLTRRGQLCWYKKEGVGLDAINDSFLLESSVYRVLKKYCRQRPYYVHLLELFLQTAYQTELGQMLDLITAPVSKVDLSHFSEERYKAIVKYKTAFYSFYLPVAAAMYMVGIDSKEEHENAKAILLEMGEYFQIQDDYLDCFGDPALTGKVGTDIQDNKCSWLVVQCLQRVTPEQRQLLEDNYGRKEPEKVAKVKELYEAVGMRAAFQQYEESSYRRLQELIEKHSNRLPKEIFLGLAQKIYKRQKGTGENLYFQGSGGGGSDYKDDDDKGTG

SEQ ID NO: 55 - DNA, 27 bp, Artificial Sequence
  Description of Artificial Sequence: Synthetic primer
gtgctaggtaactaacgtttgattttt

SEQ ID NO: 56 - DNA, 20 bp, Artificial Sequence
  Description of Artificial Sequence: Synthetic primer
tgggttcatatctggacgtt

SEQ ID NO: 57 - DNA, 23 bp, Artificial Sequence
  Description of Artificial Sequence: Synthetic primer
ggaaggggacgtaggtacataaa

SEQ ID NO: 58 - DNA, 23 bp, Artificial Sequence
  Description of Artificial Sequence: Synthetic primer
ttagaacgtgttttgttcccaat

SEQ ID NO: 59 - DNA, 20 bp, Artificial Sequence
  Description of Artificial Sequence: Synthetic primer
ccgaactgaggttgggttta

SEQ ID NO: 60 - DNA, 20 bp, Artificial Sequence
  Description of Artificial Sequence: Synthetic primer
gggggagcgaataggattag

SEQ ID NO: 61 - DNA, 21 bp, Artificial Sequence
  Description of Artificial Sequence: Synthetic primer
gcaacttcagctgtttgacct

SEQ ID NO: 62 - DNA, 23 bp, Artificial Sequence
  Description of Artificial Sequence: Synthetic primer
aaatttaacgtaacgatgagttg

SEQ ID NO: 63 - DNA, 23 bp, Artificial Sequence
  Description of Artificial Sequence: Synthetic primer
ttagaacgtgttttgttcccaat

SEQ ID NO: 64 - DNA, 24 bp, Artificial Sequence
  Description of Artificial Sequence: Synthetic primer
tggtacaagaggatttttgttgtt

SEQ ID NO: 65 - DNA, 23 bp, Artificial Sequence
  Description of Artificial Sequence: Synthetic primer
aaatttaacgtaacgatgagttg

SEQ ID NO: 66 - DNA, 22 bp, Artificial Sequence
  Description of Artificial Sequence: Synthetic primer
cgttcttctgagaaatggctta

SEQ ID NO: 67 - DNA, 20 bp, Artificial Sequence
  Description of Artificial Sequence: Synthetic primer
attagcatcagagccgcatt

SEQ ID NO: 68 - DNA, 20 bp, Artificial Sequence
  Description of Artificial Sequence: Synthetic primer
ctcgcctatcggctaacaag

SEQ ID NO: 69 - DNA, 20 bp, Artificial Sequence
  Description of Artificial Sequence: Synthetic primer
cacaagaagcaaccccttga

SEQ ID NO: 70 - DNA, 21 bp, Artificial Sequence
  Description of Artificial Sequence: Synthetic primer
cgacggaataaaggtgtacga

SEQ ID NO: 71 - DNA, 24 bp, Artificial Sequence
  Description of Artificial Sequence: Synthetic primer
tggtacaagaggatttttgttgtt

SEQ ID NO: 72 - DNA, 20 bp, Artificial Sequence
  Description of Artificial Sequence: Synthetic primer
tcagcaattgcagcgtaata

SEQ ID NO: 73 - DNA, 22 bp, Artificial Sequence
  Description of Artificial Sequence: Synthetic primer
gacgttcttgacgttttgtttg

SEQ ID NO: 74 - DNA, 1718 bp, Artificial Sequence
  Description of Artificial Sequence: Synthetic polynucleotide
atggtaccaagacgttcaggtaactataatcctagccgttgggacgtaaatttcattcaatcttattatctgattataaagaagataaacacgttattagagcttctgaattagtaacacttgttaagatggaattagaaaaagaaacagatcaaatccgtcaattagaattaattgacgatttacaacgtatgggtttatctgatcatttccaaaacgaatttaaagaaatcttatcaagtatttacttagatcatcattattacaaaaatccatttccaaaagaagagcgtgatttatactcaactagcttagcttttcgtttattacgtgaacacggttttcaagtagcacaagaagtttttgattcattcaaaaatgaagagggtgaatttaaggagagcttatctgacgatactcgtggcttattacaattatatgaagcatcattcttattaacagagggtgaaacaaccttagaaagtgcacgcgaatttgctacaaaatttttagaagaaaaagttaacgaaggtggcgttgatggtgacttattaacaagaattgcttactcattagatattcccttacattggcgcattaaacgtcctaatgccccagtttggattgaatggtatcgtaaacgtccagatatgaacccagtggttttagaattagcaattttagacttaaacattgtacaagctcaatttcaagaggaattaaaagagtcttttcgctggtggcgtaatactggttttgttgagaaattaccatttgcacgtgatcgtttagttgaatgttacttttggaacactggtattattgaaccacgtcaacacgcatcagctcgtattatgatgggtaaagtaaatgcattaattacagtaattgatgacatctatgatgtttatggaacacttgaagaattagaacaattcactgatttaattcgcagatgggacataaactcaatagatcaattaccagattatatgcaattatgttttcttgcattaaacaatttcgttgatgacacttcatacgatgttatgaaagaaaagggtgttaatgttattccttacttacgtcaatcttgggtagaccttgcagacaaatatatggtagaagcacgttggttctacggtggccataaaccatcattagaagaatacttagaaaattcttggcaatctatctcaggtccatgtatgttaactcatatattctttcgtgtaacagatagctttactaaagaaactgttgattctctttacaaatatcatgatttagttagatggtcatcattcgtgcttcgtcttgctgacgacttaggtacaagcgttgaagaagtatctcgtggtgatgtgccaaaatctttacaatgctacatgagtgattataacgctagtgaggctgaagcacgtaaacacgtaaaatggttaattgcagaagtatggaaaaagatgaatgcagaacgtgtttctaaagatagtccttttggtaaagattttataggttgtgctgttgatttaggtcgtatggctcaattaatgtatcacaatggagatggtcatggtactcaacaccctattattcatcaacaaatgacacgtactttatttgaaccattcgctggtaccggtgaaaacttatactttcaaggctcaggtggcggtggaagtgattacaaagatgatgatgataaaggaaccggttaa

SEQ ID NO: 75 - DNA, 1698 bp, Artificial Sequence
  Description of Artificial Sequence: Synthetic polynucleotide
atggtaccaagacgtactggtggctatcaacctacactttgggatttttcaacaattcaattatttgatagtgaatataaagaagaaaaacatcttatgcgtgctgctggtatgattgctcaagtgaacatgttacttcaagaagaagtagacagcatccaacgtcttgaattaattgatgacttacgtcgtttaggtatatcttgccactttgatcgtgaaattgtagagattttaaacagtaaatactacaccaacaatgaaattgatgaatcagatttatacagtacagcacttagattcaaacttttacgtcaatatgattttagcgttagccaagaagtttttgattgttttaaaaatgacaaaggtacagatttcaaaccatcattagttgacgatacacgtggcttattacaattatatgaagcatcatttttatcagctcagggtgaagaaactttacatttagcacgtgattttgctactaaattcttacataaaagagttttagtagataaagatatcaatttattatctagtatcgagcgtgctttagaattaccaacacactggcgtgtacaaatgcctaacgctagatcattcatcgacgcatataaaagaagaccagacatgaaccctacagtattagagttagcaaaacttgactttaacatggttcaagcacagttccaacaagaattaaaagaagccagtcgctggtggaactctacaggattagtacatgaattaccatttgtacgtgatcgtattgtggaatgttattattggactactggtgtagtagaacgtcgtgaacacggttacgaacgtattatgttaacaaaaattaacgctttagttacaacaatcgatgatgtttttgacatttatggtactttagaagaattacaactttttacaactgctattcaaagatgggacattgagtctatgaaacaacttccaccctatatgcaaatctgctacttagctttattcaacttcgtaaatgagatggcttacgatacattacgtgataaaggttttaatagtactccatatttacgcaaagcctgggtagacttagtagaaagctacttaattgaagctaaatggtattatatgggtcacaaaccaagtttagaagagtacatgaaaaactcatggatttctatcggaggtattccaattttatcacatttattctttcgtttaacagacagtatcgaagaagaagacgctgaatcaatgcataaatatcacgatatagtacgtgcctcttgtactattttacgtttagctgatgatatgggtacatcattagatgaagttgaacgtggcgatgttcctaaatctgtacaatgctatatgaatgagaaaaacgcctctgaagaagaagcacgtgaacatgttcgtagtttaattgatcagacatggaagatgatgaataaagaaatgatgacttcatcattttcaaaatacttcgtacaagtgtctgcaaatcttgctcgtatggcacaatggatatatcaacatgaaagtgatggtttcggtatgcaacactctttagttaacaaaatgcttcgtggtttactttttgaccgttatgaaggtaccggtgaaaacttatactttcaaggctcaggtggcggtggaagtgattacaaagatgatgatgataaaggaaccggttaa

SEQ ID NO: 76 - DNA, 1788 bp, Artificial Sequence
  Description of Artificial Sequence: Synthetic polynucleotide
atggtaccacgtcgcatgggtgattttcattcaaacttatgggatgatgatgtaattcaatctttacccacagcttacgaagaaaaatcttatcttgaacgtgctgagaagttaattggagaagttgaaaatatgttcaacagtatgagtttagaagatggtgaacttatgagtccattaaatgatttaattcaacgcctttggattgttgattctttaggtagattaggtatccatcgtcactttaaagatgagattaaaagtgctttagattatgtttacagttactggggtgaaaacggaataggttgtggtcgtgaaagtgctgtaactgatttaaacagtacagctttaggctttcgtacacttcgtttacacggttatccagtttcatctgatgtatttaaagcatttaaaggtcaaaatggtcaattcagttgttcagaaaatatccaaacagatgaagaaattcgtggtgttcttaacttatttagagccagtttaatagccttccctggtgagaaaataatggacgaagctgaaatcttctctacaaaatacttaaaggaagcattacaaaagatcccagttagttcattatcacgtgaaatcggtgatgtacttgaatatggatggcatacatacttaccacgtttagaagcacgtaactatattcatgttttcggacaagatacagagaatacaaaaagttatgtaaaatcaaagaaacttttagaattagctaaattagaatttaacatttttcagagcttacaaaaacgtgaattagaaagccttgttcgttggtggaaagaatctggatttcctgaaatgacattctgtagacacagacacgtggaatattacacacttgcatcatgtattgcattcgaacctcagcatagtggttttcgtttaggttttgctaaaacatgtcaccttataacagttttagatgacatgtatgacactttcggcaccgtagacgaattagagttatttacagcaactatgaaacgttgggacccaagttcaattgactgccttccagaatacatgaaaggagtttacattgctgtgtatgatacagttaatgaaatggctcgtgaagctgaggaagctcaaggtcgcgatacacttacatacgctcgtgaggcctgggaggcttatatagattcttatatgcaagaagctcgctggattgctactggatacttaccttctttcgatgaatattatgaaaatggtaaggtttcatgtggtcaccgtatatctgctttacaaccaattcttactatggatattccatttccagatcacattttaaaggaagttgactttccttctaaacttaatgacttagcttgtgctatcttacgccttcgcggtgatactcgttgttacaaagcagaccgtgcacgtggtgaagaggctagttctatttcttgttatatgaaagataatccaggtgtttctgaagaagatgccttagatcatattaacgcaatgatcagtgatgttattaagggcttaaactgggaattacttaaacccgacattaacgtacctatttctgctaagaaacatgctttcgacattgctcgtgcttttcactacggttataaatatcgtgatggctattcagttgctaatgttgaaacaaaatctttagttacacgtactttacttgaatcagttccattaggtaccggtgaaaacttatactttcaaggctcaggtggcggtggaagtgattacaaagatgatgatgataaaggaaccggttaa

SEQ ID NO: 77 - DNA, 1773 bp, Artificial Sequence
  Description of Artificial Sequence: Synthetic polynucleotide
atggtaccacgtagagttggtaattatcattctaatctttgggatgatgattttatacaaagtttaatttctacaccttacggtgctcctgactaccgtgaacgcgctgatcgtcttattggtgaagtaaaagatattatgtttaatttcaaatctttagaggatggtggtaatgacttattacaacgtttacttttagttgatgacgtagaacgtttaggcattgatcgtcatttcaaaaaggaaattaagactgcattagattatgtaaatagttattggaatgaaaaaggaattggttgtggtcgtgagtctgtagttacagacttaaattcaactgctttaggccttcgtaccttaagattacatggttatactgttagctctgacgttttaaatgtttttaaagataaaaatggtcaattttcatctacagctaatattcaaattgaaggtgaaattcgtggtgttttaaatctttttcgtgcctctcttgtagcttttccaggtgagaaagtgatggatgaggctgaaactttttcaacaaaatatcttcgtgaagcattacagaaaattcctgccagttcaattttatcattagaaatacgtgatgtattagaatatggatggcatactaatttaccacgtttagaagcacgtaattacatggatgttttcggtcagcacaccaagaacaaaaatgcagccgaaaaattacttgaattagcaaaattagagttcaatatctttcacagcttacaagaacgtgaattaaagcacgtttcaagatggtggaaagactctggtagtccagagatgactttctgtcgccaccgccatgtggaatattatgctttagcttcttgtattgctttcgaaccccagcacagtggtttccgtttaggttttactaaaatgagtcatttaatcacagtgttagatgatatgtatgatgtattcggtacagttgatgaattagagttatttaccgccactattaaacgttgggacccttctgctatggaatgtttaccagagtacatgaaaggtgtttacatgatggtttatcatacagttaacgaaatggctcgtgtggcagaaaaggctcaaggtagagacacattaaactatgctcgtcaagcctgggaagcatgttttgactcttatatgcaagaagcaaaatggattgcaacaggttacttacctacattcgaggaatatttagaaaatggtaaagtgagttcagcacatcgtccttgtgcattacaacctattttaactcttgatattccatttcccgatcatattcttaaagaagtggatttcccaagcaaacttaatgacttaatttgtattatcttacgtcttagaggagacacacgttgctataaagcagaccgtgcccgtggtgaagaagcatcatcaatatcttgttatatgaaagataacccaggtttaactgaagaagatgctttaaaccacattaactttatgattcgtgacgcaatccgcgaattaaactgggagttacttaaaccagataatagtgttccaattacttcaaagaaacatgcttttgatatttcacgtgtgtggcaccacggataccgttatcgtgatggttacagctttgcaaacgtggaaactaaaagtcttgtaatgcgtactgtaatagaaccagtaccattaggtaccggtgaaaacttatactttcaaggctcaggtggcggtggaagtgattacaaagatgatgatgataaaggaaccggttaa

SEQ ID NO: 78 - DNA, 1710 bp, Artificial Sequence
  Description of Artificial Sequence: Synthetic polynucleotide
atggtaccacgtcgttcaggagattatcaaccaagtttatgggactttaattacattcaatctttaaacacaccttacaaagaacaacgtcattttaatcgtcaagctgagttaattatgcaagttcgtatgttattaaaggtaaaaatggaagcaattcaacaattagagttaatagatgatttacagtacttaggattatcatatttctttcaagacgaaattaaacaaatcttaagctctattcacaatgaacctcgttattttcataataatgacctttatttcactgctttaggttttagaattttacgtcaacatggttttaatgtttcagaagacgtatttgactgctttaaaatcgaaaaatgttctgactttaatgctaacttagctcaggacacaaagggtatgttacaattatatgaagctagtttcttattaagagaaggagaagatacacttgaattagctcgtcgttttagtacacgttctttacgtgaaaaatttgatgaaggtggtgacgagatagatgaagatttaagtagttggattcgtcattctttagatttaccattacactggcgtgttcaaggtttagaagctcgttggtttttagatgcctatgctcgtcgtccagatatgaaccctcttattttcaaattagctaaattaaattttaacattgttcaagctacataccaagaagaattaaaagacatctctcgttggtggaacagtagttgtttagcagagaaattacccttcgttcgcgatcgtattgtagaatgtttcttctgggctattgctgctttcgaaccacaccaatactcatatcaacgtaaaatggccgctgtaattattacatttattactattattgatgatgtttacgatgtatatggtactattgaagaattagagttattaacagatatgattcgtagatgggataataagagtattagtcaacttccttactatatgcaagtttgttatttagctctttataacttcgtaagtgaacgcgcatacgacatcttaaaagatcaacactttaacagtattccataccttcaaagaagttgggtttcattagttgagggatacttaaaagaagcatattggtactataacggttacaaaccaagtcttgaagaatatcttaataatgcaaaaattagtattagtgcacccaccattatttcacaattatactttactttagcaaatagtatcgacgaaactgccattgaaagtttataccaatatcacaacattttatacttatcaggtactatcttacgtttagctgatgatttaggaacttcacaacatgaattagaacgtggtgatgttcccaaagctattcaatgttatatgaatgatacaaatgcatcagaaagagaagctgtagaacatgttaaatttcttattcgtgaagcctggaaagaaatgaatacagttactaccgcatcagattgtccttttacagacgatcttgttgccgccgcagctaatttagctcgtgctgctcaattcatttacttagatggtgatggtcatggtgtacaacatagcgaaattcatcagcaaatgggcggtcttctttttcaaccatacgttggtaccggtgaaaacttatactttcaaggctcaggtggcggtggaagtgattacaaagatgatgatgataaaggaaccggttaa

SEQ ID NO: 79 - DNA, 1785 bp, Artificial Sequence
  Description of Artificial Sequence: Synthetic polynucleotide
atggtaccacgcagaattggtgattaccatagtaacatttgggatgatgattttatccagtcactttctaccccttatggtgaaccatcttaccaagaaagagctgaacgtcttattgtagaagtgaaaaagattttcaacagtatgtacttagatgacggtcgtttaatgagttcttttaatgacttaatgcaacgtttatggattgtagactcagtagaacgtttaggtattgcccgtcacttcaaaaatgaaattacatctgcccttgactatgtttttcgttattgggaagaaaacggtataggttgtggtcgtgattctattgtaactgacttaaatagcacagctttaggttttcgtacacttcgtttacacggttatacagtttctccagaggttttaaaagcatttcaagatcaaaatggtcaattcgtttgttcaccaggacaaacagaaggtgaaattcgttcagttttaaatttatatcgtgcaagtttaattgcctttccaggcgaaaaagttatggaagaagcagaaatcttctctactcgctatttaaaagaagctcttcaaaagattccagttagcgcattatcacaagaaatcaaatttgttatggaatatggatggcatacaaatttacctagattagaagcacgtaactatattgatactttagaaaaggatacatcagcttggttaaacaaaaatgcaggtaaaaagttattagaattagctaaattagaatttaacatctttaactcattacaacaaaaagaattacaatacttacttcgctggtggaaagaatctgacttacctaaattaacctttgcacgtcatagacacgttgaattttacacattagcttcttgtattgctattgatcccaaacattcagcattccgtttaggattcgctaaaatgtgtcacttagttacagttcttgacgatatttatgatactttcggtactattgatgaacttgagttatttacttctgcaattaaacgttggaatagttctgaaattgaacacttaccagaatatatgaaatgcgtgtatatggttgtttttgaaactgttaatgaattaactcgtgaagctgagaaaacacaaggacgtaacactttaaactatgttcgtaaagcatgggaagcatattttgattcttatatggaggaagcaaagtggatctcaaacggatatttaccaatgtttgaagaataccacgaaaatggtaaagtgtcatctgcataccgtgtagcaacattacaaccaattttaactttaaacgcttggttacccgactacattcttaaaggaattgatttcccaagtcgttttaacgatttagctagttcattcttacgtttacgtggcgatactcgctgttacaaagctgaccgtgatcgtggtgaagaagctagctgcatttcttgttacatgaaagataatccaggttctaccgaagaagatgcacttaatcacattaacgctatggtaaatgacatcattaaagaattaaactgggaattattacgcagtaatgataatattcctatgttagctaaaaagcacgcttttgatattactcgtgcacttcaccacttatacatttatcgcgatggtttcagtgttgctaataaagaaactaaaaagttagttatggagacattacttgaatcaatgttatttggtaccggtgaaaacttatactttcaaggctcaggtggcggtggaagtgattacaaagatgatgatgataaaggaaccggttaa

SEQ ID NO: 80 - DNA, 2487 bp, Artificial Sequence
  Description of Artificial Sequence: Synthetic polynucleotide
atggtaccacaatctgctgaaaagaacgactctttatcaagttctacattagttaagagagaatttccacccggtttctggaaagacgacttaatcgacagtttaacttcaagtcacaaagtagctgctagcgatgaaaaacgtatcgaaaccttaatttcagaaattaagaatatgtttcgttgtatgggttatggtgagacaaatccatcagcttatgatactgcttgggtagctcgcatcccagcagttgatggatcagataatcctcactttccagagactgtggaatggatcttacaaaatcaattaaaagatggttcttggggtgaaggtttttacttccttgcttatgatcgcattttagccactttagcttgtattatcacacttacactttggcgtactggagaaacacaagtacagaaaggtatcgaatttttccgcactcaagcaggtaaaatggaagatgaagcagattcacaccgtccaagtggttttgagattgtatttcctgctatgttaaaagaggctaagattttaggcttagatttaccttatgatcttccttttcttaaacaaattattgaaaagagagaagctaagttaaaacgtattcctacagatgttttatatgctttaccaactactttactttattcattagaaggtttacaagaaatagtagactggcaaaaaatcatgaaattacaaagtaaagatggtagtttcttatcttctcctgcctcaacagcagcagtatttatgagaacaggtaacaaaaagtgtttagatttcttaaatttcgtgcttaaaaagttcggtaatcatgttccatgccactatcctttagacctttttgagcgtctttgggcagttgatactgttgaaagattaggtattgaccgtcattttaaagaagaaataaaagaggctttagactatgtgtattcacactgggacgaacgtggtattggttgggctcgtgaaaaccccgttccagatattgacgatacagcaatgggtcttcgtattttacgtcttcatggttacaatgttagcagcgatgttcttaaaacatttcgtgatgaaaatggtgagttcttttgctttttaggacaaacacaaagaggtgtgactgatatgttaaatgttaatcgttgtagccatgtatctttccctggtgaaactataatggaagaggcaaaattatgtactgaacgttacttacgcaacgcattagaaaatgtagacgcttttgataagtgggcatttaagaaaaacattcgtggtgaggtagaatatgctcttaaatatccttggcataaatcaatgccacgtttagaagcacgttcatatattgaaaattacggtccagatgatgtttggttaggtaaaactgtttatatgatgccttacatttcaaatgaaaagtacttagagttagctaaacttgattttaacaaagttcagtcaatccaccagacagaacttcaagacttacgccgttggtggaaaagttctggttttacagatttaaactttacaagagaacgtgttactgaaatttacttttcacctgcatcttttatcttcgaaccagaatttagtaaatgtcgtgaggtttatacaaaaacttctaattttactgtaattttagacgatttatatgacgctcatggctctttagatgacttaaaactttttacagagagtgttaaacgttgggatttatctttagttgaccaaatgccccagcagatgaaaatctgttttgtaggtttctataatacattcaacgatattgctaaagaaggtagagaacgtcaaggtcgtgatgttttaggttatattcaaaacgtatggaaagtacaacttgaagcatatactaaagaagcagaatggtcagaagcaaaatatgttcctagttttaacgaatacattgaaaatgcttcagtttcaattgccttaggtacagtagtacttatcagtgctttatttaccggagaagttttaacagatgaagttttatctaaaattgaccgtgaaagtagattcttacagttaatgggcttaactggacgtttagtaaatgatactaaaacatatcaagctgagcgtggtcaaggtgaagttgctagtgcaattcaatgttatatgaaagaccaccctaaaattagtgaagaagaagcattacaacatgtatattctgtaatggaaaatgcattagaagaattaaatcgtgagttcgttaacaacaaaattccagacatctataaacgtcttgttttcgaaactgcacgtataatgcaattattttacatgcaaggtgatggtttaacattaagtcacgatatggaaattaaagagcacgtaaagaattgtttattccagccagtagctggtaccggtgaaaacttatactttcaaggctcaggtggcggtggaagtgattacaaagatgatgatgataaaggaaccggttaa

SEQ ID NO: 81 - DNA, 2496 bp, Artificial Sequence
  Description of Artificial Sequence: Synthetic polynucleotide
atggtaccatcttcatcaacaggcacttcaaaagtagtaagcgaaacatcttcaactattgtagacgatattccacgtctttcagcaaattatcatggtgatttatggcatcacaacgtaattcagactttagaaacaccatttagagaaagttcaacttatcaagagcgtgcagatgaattagtagtgaaaatcaaagatatgttcaatgcattaggtgacggtgacatctcaccttcagcttatgatactgcatgggtagctcgtgttgctaccatttcttctgatggtagcgaaaaaccacgttttcctcaagctcttaattgggtttttaacaatcaattacaagatggatcatggggtattgaatcacattttagtttatgcgatcgtttacttaatactacaaattcagttattgctttatcagtatggaaaactggtcactcacaggttcaacaaggtgccgaatttattgctgaaaatttacgtcttttaaatgaagaagacgaattaagtcctgattttcaaattatcttcccagctttattacagaaagccaaggctttaggaatcaatttaccctatgatttaccattcatcaaatatcttagtacaacacgcgaagctcgtttaacagatgtgtcagctgctgctgacaacataccagccaatatgcttaatgcacttgaaggtttagaagaagtgattgattggaataaaatcatgcgttttcaatctaaagatggttcatttttatcttctccagctagtacagcctgtgttttaatgaatacaggtgatgaaaaatgtttcacattcttaaataacttattagataaattcggcggttgtgttccatgtatgtatagcattgatttattagaacgtttatctttagtggacaacattgaacacttaggtattggtcgtcactttaaacaagaaatcaaaggtgcattagattatgtatatcgtcattggtctgaacgcggtatcggttggggtagagactctttagttccagatttaaacaccacagctttaggtttacgcacattaagaatgcacggttataacgtgtctagtgatgtacttaacaatttcaaagacgaaaatggtcgtttctttagtagtgctggtcaaacacacgtagagttacgttctgttgtaaatctttttcgcgcctcagatttagcctttccagacgaacgtgcaatggatgatgctcgtaaattcgcagaaccatatttacgtgaagcattagctacaaaaatatcaacaaatacaaagttattcaaagaaattgaatatgttgttgaatacccttggcacatgtcaattccacgtttagaagctcgtagttatattgacagttatgatgataattatgtatggcaacgtaagactttatatcgtatgccatcattaagtaattcaaaatgtttagaacttgctaaattagatttcaatattgttcaatctttacaccaagaagaacttaaacttttaactcgttggtggaaagaatctggtatggcagacataaatttcacccgccatcgtgtagctgaagtttacttttctagtgctacatttgagccagaatatagtgctactcgtattgcattcacaaaaattggttgcttacaagtacttttcgatgatatggctgacattttcgccactttagatgagttaaaaagttttactgaaggtgttaaacgctgggacacatcattattacatgaaattcccgaatgtatgcaaacttgttttaaagtatggtttaaacttatggaagaagtaaacaacgacgtagtaaaagttcaaggaagagatatgttagcacatattcgtaaaccctgggaattatactttaattgttatgttcaagaacgtgaatggttagaagctggttatattcctacattcgaagaatatcttaaaacttatgctattagtgtaggccttggtccttgtaccttacaacctattcttttaatgggtgagttagttaaagatgatgtagtagaaaaagttcattacccttctaacatgttcgaattagtttctttaagctggcgtttaactaatgataccaaaacatatcaagcagaaaaagtacgcggtcaacaagctagtggcattgcctgttatatgaaagacaatccaggtgctactgaagaagatgctattaaacacatttgtcgtgttgttgatcgtgcattaaaagaagcaagtttcgaatatttcaagccttcaaatgacattcctatgggttgtaaatcttttatctttaacttacgtttatgtgtacaaattttctataaattcattgatggttatggtatcgcaaacgaagaaattaaggactacattcgtaaggtttatattgatccaattcaagttggtaccggtgaaaacttatactttcaaggctcaggtggcggtggaagtgattacaaagatgatgatgataaaggaaccggttaa

SEQ ID NO: 82 - DNA, 1191 bp, Artificial Sequence
  Description of Artificial Sequence: Synthetic polynucleotide
atggtaccacacaagttcacaggtgttaacgctaaattccagcaaccagcattaagaaatttatctccagtggtagttgagcgcgaacgtgaggaatttgtaggattctttccacaaattgttcgtgacttaactgaagatggtattggtcatccagaagtaggtgacgctgtagctcgtcttaaagaagtattacaatacaacgcacctggtggtaaatgcaatagaggtttaacagttgttgcagcttaccgtgaactttctggaccaggtcaaaaagacgctgaaagtcttcgttgtgctttagcagtaggatggtgtattgaattattccaagcctttttcttagttgctgacgatataatggaccagtcattaactagacgtggtcaattatgttggtacaagaaagaaggtgttggtttagatgcaataaatgattcttttcttttagaaagctctgtgtatcgcgttcttaaaaagtattgccgtcaacgtccatattatgtacatttattagagctttttcttcaaacagcttaccaaacagaattaggacaaatgttagatttaatcactgctcctgtatctaaggtagatttaagccatttctcagaagaacgttacaaagctattgttaagtataaaactgctttctattcattctatttaccagttgcagcagctatgtatatggttggtatagattctaaagaagaacatgaaaacgcaaaagctattttacttgagatgggtgaatacttccaaattcaagatgattatttagattgttttggcgatcctgctttaacaggtaaagtaggtactgatattcaagataacaaatgttcatggttagttgtgcaatgcttacaaagagtaacaccagaacaacgtcaacttttagaagataattacggtcgtaaagaaccagaaaaagttgctaaagttaaagaattatatgaggctgtaggtatgagagccgcctttcaacaatacgaagaaagtagttaccgtcgtcttcaagagttaattgagaaacattctaatcgtttaccaaaagaaattttcttaggtttagctcagaaaatatacaaacgtcaaaaaggtaccggtgaaaacttatactttcaaggctcaggtggcggtggaagtgattacaaagatgatgatgataaaggaaccggttaa

SEQ ID NO: 83 - DNA, 1728 bp, Artificial Sequence
  Description of Artificial Sequence: Synthetic polynucleotide
atggtaccatcattaactgaagaaaaaccaattcgcccaatcgcaaactttcctccaagcatttggggagatcaattcttaatttacgaaaaacaagtagaacaaggtgttgaacagattgttaacgaccttaagaaagaagtgcgccaacttttaaaagaggctttagatattccaatgaaacacgcaaaccttttaaaacttattgacgaaattcaacgtcttggtattccatatcactttgaacgtgaaattgatcatgcattacaatgtatctatgaaacttatggtgataattggaatggtgatcgttcttcattatggttccgtttaatgcgtaaacaaggttattatgttacatgtgacgtgtttaacaattacaaagataaaaatggtgcatttaaacaatctttagctaatgatgttgaaggtttattagaattatatgaagctacttcaatgcgtgttccaggtgaaattattcttgaagatgcattaggttttacacgttctcgtttatctattatgacaaaagacgcatttagtacaaatcctgctttatttactgaaattcagcgtgcccttaaacagcctttatggaaacgtttaccaagaattgaagctgctcaatatattccattttatcaacaacaagattctcacaataagacattacttaaattagccaaattagaatttaatcttttacaatcattacataaagaagaattaagtcatgtgtgtaaatggtggaaagcatttgatattaagaagaatgctccatgtttacgtgatagaattgtagagtgttacttttggggccttggtagtggttacgagccacaatattcacgtgctcgtgtattctttacaaaagctgttgcagttattactttaattgacgatacctatgatgcatacggaacctatgaggagcttaaaattttcactgaagctgtagaacgttggtctataacttgtttagatactttaccagaatatatgaaacccatctacaaattattcatggacacatacactgaaatggaagaatttttagcaaaagaaggtcgcacagacctttttaactgtggtaaagaatttgttaaagagtttgttcgtaacttaatggtagaagctaagtgggctaatgaaggtcacattcctactacagaagagcacgatccagtagtaataattacaggtggagcaaacttacttaccacaacttgttacttaggtatgtctgacatttttacaaaagaatcagtagagtgggcagtatctgcaccaccattattccgttattctggcatacttggtcgtcgtcttaatgatttaatgactcataaagctgaacaagagcgtaaacactcatcaagtagtttagaaagctatatgaaggaatataacgttaacgaagagtatgctcaaacacttatttacaaagaggttgaagacgtttggaaggacattaaccgtgaatacttaacaactaaaaacattccacgtcctcttttaatggctgtaatatacttatgtcaattcttagaagtacaatacgctggaaaagataactttacacgtatgggtgatgaatataaacacttaataaagagtttattagtttatcctatgtcaataggtaccggtgaaaacttatactttcaaggctcaggtggcggtggaagtgattacaaagatgatgatgataaaggaaccggttaa

SEQ ID NO: 84 - DNA, 2541 bp, Artificial Sequence
  Description of Artificial Sequence: Synthetic polynucleotide
atggtaccagcaggtgtatcagctgtgtcaaaagtttcttcattagtatgtgacttaagtagtactagcggcttaattcgtagaactgcaaatcctcaccctaatgtatggggttatgacttagttcattctttaaaatctccatatattgatagtagctatcgtgaacgtgctgaagtgcttgtaagtgaaataaaagctatgttaaatccagcaattactggagatggtgaatcaatgattacaccttcagcttatgacactgcttgggttgcacgtgtaccagcaattgatggtagcgcacgtccacaatttccacaaacagtagattggattttaaagaatcaattaaaagatggttcttggggtattcaatcacactttttactttcagaccgtttattagctactcttagctgtgttttagttttacttaaatggaatgttggtgatttacaggttgagcaaggtattgagtttattaagtcaaaccttgaattagtaaaagatgaaactgatcaagattctttagtgactgattttgagattattttccctagcttacttcgtgaggcccaaagtttacgtttaggtcttccatacgatttaccttacatccacttattacaaacaaaacgtcaggaacgtttagcaaaattaagccgtgaagaaatatatgcagttccaagtccacttttatattctttagagggtattcaagatattgttgagtgggaacgtattatggaagtacaatctcaggatggatcatttttaagttctccagcatcaaccgcatgtgtttttatgcatacaggtgacgctaagtgtttagaatttcttaacagtgtaatgattaagtttggtaattttgtaccatgcctttatcctgtagatttattagaacgtttacttatagtagataatatagttcgtcttggtatttaccgtcacttcgaaaaagaaattaaagaagcattagattatgtatatcgccattggaatgaacgtggtattggttggggtcgtttaaatccaattgctgacttagaaacaactgctttaggttttcgtttattacgtttacaccgttataatgtatctccagcaatctttgataatttcaaagatgccaatggcaaattcatttgtagcactggtcagtttaataaggatgtggcttcaatgttaaacttataccgtgcatcacaattagcattcccaggcgaaaacattttagatgaagctaaatcttttgccaccaaatacttacgtgaagcccttgaaaaatctgaaacttcatcagcttggaacaataaacagaatttaagtcaagaaatcaagtatgcattaaaaacttcatggcacgcttctgtaccacgtgttgaagcaaaacgttattgtcaagtttatcgtcctgattacgctcgtattgctaagtgtgtatacaaattaccatacgttaacaacgaaaaattcttagaattaggtaaattagattttaacatcattcaatcaattcatcaagaagaaatgaaaaatgtgacaagttggtttcgtgattctggcttaccattatttactttcgctcgcgaacgtcctttagaattttacttcttagttgctgctggtacttatgaacctcaatatgctaaatgtcgtttcttattcacaaaagtagcttgtcttcaaacagtattagacgatatgtacgatacttacggtactttagacgaattaaaactttttaccgaggctgtgcgtcgttgggatttatcttttacagaaaatttacctgactatatgaaattatgttatcaaatctattatgacatcgttcatgaagtggcttgggaagctgaaaaagaacaaggtagagaattagtgtcattcttccgtaaaggctgggaagactacttattaggttactatgaagaagcagaatggttagcagcagaatacgttccaacattagatgaatacattaaaaacggtattacatcaatcggccaacgtatcttattactttcaggtgtgttaattatggatggccaacttttatcacaagaagcattagaaaaagttgattaccctggtcgtcgtgttttaactgagttaaactcacttattagccgtttagctgacgacactaaaacttataaagcagaaaaagctcgtggagaattagcctcatcaattgaatgctacatgaaagatcatcctgaatgtacagaagaagaagccttagaccacatttattctattcttgaaccagccgtaaaagaattaactcgtgaatttcttaaaccagacgacgttccatttgcttgtaaaaagatgttattcgaagaaactcgtgttacaatggtgatctttaaagatggtgatggttttggtgtatctaagttagaagttaaagatcacatcaaagaatgcttaattgaaccattaccattaggtaccggtgaaaacttatactttcaaggctcaggtggcggtggaagtgattacaaagatgatgatgataaaggaaccggttaa

SEQ ID NO: 85 - DNA, 933 bp, Artificial Sequence
  Description of Artificial Sequence: Synthetic polynucleotide
atggtaccaactatgatgaatatgaattttaagtactgtcacaagattatgaagaaacattcaaaatcattcagttatgcttttgacttattaccagaagaccaacgtaaagctgtttgggcaatttacgccgtgtgccgcaaaattgatgattctattgatgtatatggtgatattcaattcttaaatcagattaaagaagacatacaaagtattgaaaaatatccatacgaacatcatcattttcaatctgacagacgtattatgatggccttacagcatgttgctcagcataaaaacattgcatttcaatcattctacaatttaattgacacagtatataaagatcaacactttacaatgtttgaaacagatgctgaactttttggttattgttacggtgtagctggtactgtgggtgaagttttaactcctatattatctgatcacgaaacacatcaaacttatgacgttgcccgtcgtttaggagagtcattacagttaatcaatattcttagagatgtaggtgaagactttgacaacgaacgtatttacttctctaaacaacgtttaaaacaatacgaagtagatattgcagaagtgtaccaaaatggtgtaaacaatcactatattgatttatgggaatattacgctgcaattgctgaaaaggattttcaagatgttatggaccaaattaaagttttctctattgaagctcagccaattattgagttagctgcacgtatttatatcgaaattttagatgaagtacgtcaagctaactacacattacatgaacgtgtttttgtagataaacgtaaaaaggctaaactttttcacgaaaataaaggtaccggtgaaaacttatactttcaaggctcaggtggcggtggaagtgattacaaagatgatgatgataaaggaaccggttaa

SEQ ID NO: 86 - DNA, 1596 bp, Artificial Sequence
  Description of Artificial Sequence: Synthetic polynucleotide
atggtaccaaaaattgctgttattggtgctggtgttaccggattagctgctgctgctcgtattgctagccaaggtcatgaagttacaatcttcgaaaaaaacaataatgtaggtggtcgtatgaatcaattaaaaaaagatggttttacattcgatatgggacctacaattgttatgatgccagatgtatataaagatgtatttactgcttgcggtaaaaactatgaagattatatagagttacgtcaacttcgttacatttatgacgtatatttcgatcacgatgatcgtattactgttccaactgatttagctgaattacaacaaatgttagaatcaattgaacctggtagtacacacggatttatgtcatttttaacagatgtgtacaagaaatatgaaatcgctcgcagatatttcttagaacgtacttaccgtaaaccatcagacttctacaatatgacctctttagtacaaggtgctaaacttaaaactttaaatcacgctgatcaacttatcgaacactacattgataacgaaaagattcaaaaacttttagcattccaaactctttatatcggcattgatccaaagcgtggtcctagtttatatagtattattcctatgattgaaatgatgttcggtgtacattttatcaaaggtggtatgtatggtatggctcaaggattagctcaacttaacaaagatttaggtgttaatattgaattaaatgctgaaattgaacaaatcattatcgatcctaaattcaaacgcgcagatgcaattaaagttaatggtgacattcgcaaatttgataagattttatgtactgctgactttccttcagttgccgaatcacttatgccagatttcgcacctatcaaaaagtaccctccacataaaattgcagatttagattattcttgttcagcttttcttatgtatattggtattgacatcgacgtaactgaccaagttcgtttacataacgtaatttttagcgacgattttcgtggaaatattgaagaaattttcgaaggtcgcttaagttacgacccatcaatctatgtttatgtaccagctgtagccgataaatctttagctcctgaaggtaaaacaggcatttatgtgttaatgcctactcctgaacttaaaacaggatcaggtattgactggtcagatgaggctttaactcaacaaattaaagaaatcatttatcgtaaattagcaacaattgaagtatttgaagacattaaatcacacattgtatcagaaacaatttttactcctaatgactttgaacaaacctatcacgctaaatttggttctgctttcggtttaatgcccaccttagcacaatctaattattacagacctcaaaatgtgtcacgtgattataaagacttatatttcgcaggtgcatcaacacatccaggtgctggagttccaattgtattaacaagtgccaagataacagtagacgaaatgattaaagatattgagcgtggtgtgggtaccggtgaaaacttatactttcaaggctcaggtggcggtggaagtgattacaaagatgatgatgataaaggaaccggttaa

SEQ ID NO: 87 - DNA, 978 bp, Artificial Sequence
  Description of Artificial Sequence: Synthetic polynucleotide
atggtaccagcatttgacttcgatggttacatgcttcgtaaagctaaatctgtaaataaagctcttgaagctgcagtacaaatgaaagaaccattaaaaattcatgaaagtatgcgttattctttattagctggtggtaaacgtgtacgtccaatgttatgtattgcagcttgtgaattagttggtggtgacgaaagtactgctatgcctgctgcttgcgctgtagaaatgattcatactatgagtttaatgcatgatgatttaccatgtatggataatgacgatttacgtcgtggtaaaccaacaaaccacatggcatttggtgaaagtgtagcagtattagcaggtgatgcattattatcttttgcttttgaacatgtagcagcagcaacaaaaggtgctcctccagaacgtattgttagagttttaggtgaacttgcagtttctattggttcagaaggtttagttgctggacaagtagttgacgtttgttctgaaggtatggctgaggttggtttagatcatttagaatttattcatcaccacaaaactgctgctttattacaaggttctgtagtattaggtgcaatattaggtggtggaaaagaagaagaggtagcaaaacttcgtaaattcgctaactgcattggtttacttttccaagtagtagatgatattcttgatgtaacaaaatcatctaaagaattaggtaaaacagcaggtaaagatttagttgctgataaaactacttatccaaaattaatcggtgttgagaaaagtaaagagttcgcagaccgtttaaatcgtgaagctcaagaacaacttcttcattttcatccacatagagcagcacctttaatcgctttagcaaactatattgcttatcgtgataatggtaccggtgaaaatttatattttcaaggttcaggtggcggaggttctgattataaagatgatgatgataaaggaaccggttaa

SEQ ID NO: 88 - DNA, 891 bp, Artificial Sequence
  Description of Artificial Sequence: Synthetic polynucleotide
atggtaccaagtcaaccttactgggcagcaattgaggcagatattgaacgttacttaaaaaaatcaattacaattcgtccaccagaaactgtatttggtccaatgcaccacttaacttttgctgcaccagctacagctgctagtactttatgtttagcagcatgtgaacttgtaggtggtgatcgtagtcaagctatggctgcagcagcagcaatccatcttgttcatgcagctgcttatgtacatgaacatttaccattaactgatggtagtcgtccagtaagtaaaccagctatccaacataaatatggtccaaatgtagaattacttacaggtgacggtattgtaccatttggttttgaattattagcaggttctgttgatccagcacgtacagatgatccagaccgtattttacgtgtaataattgaaataagtcgtgctggtggtccagaaggtatgattagtggtttacatcgtgaagaagagattgtagatggtaatacttctcttgattttattgaatacgtttgcaaaaaaaaatatggtgaaatgcacgcatgtggtgctgcatgcggtgcaattttaggtggtgcagctgaagaagaaattcaaaaacttcgtaacttcggattatatcaaggaactttacgtggtatgatggagatgaaaaactcacaccaacttattgacgaaaatatcattggcaaacttaaagaattagctttagaagaattaggtggatttcatggtaaaaatgctgaattaatgtctagtttagtagcagaaccatcattatatgctgctggtaccggtgaaaatttatactttcaaggttctggtggtggtggcagtgattataaagacgatgatgacaaaggaaccggttaa

SEQ ID NO: 89 - DNA, 1080 bp, Artificial Sequence
  Description of Artificial Sequence: Synthetic polynucleotide
atggtaccacttttatctaacaaattaagagagatggttttagcagaagttcctaaattagcatctgctgctgaatatttctttaaacgtggtgttcagggtaaacaattccgttcaacaattttattattaatggcaacagctcttgacgttcgtgttccagaagcattaattggtgaatctactgatattgtaacatctgaattacgtgtacgtcaacgtggcattgctgaaattacagaaatgattcatgtagcatcacttcttcacgatgacgttcttgacgatgctgatactcgtcgtggtgttggtagtcttaatgttgtaatgggaaacaaaatgtcagttttagcaggtgacttcttactttctcgtgcttgtggtgctcttgcagctcttaaaaacacagaagttgtagcattattagctacagcagtagaacacttagttactggtgagacaatggaaataacttcatcaactgaacaacgttattctatggattactacatgcagaaaacttattacaaaactgcttcattaatttcaaattcatgtaaagcagttgctgtattaacaggtcaaacagctgaagttgcagtattagcttttgaatatggtcgtaatttaggtttagctttccagttaattgacgacattttagatttcacaggcacatctgctagtttaggaaaaggttctttatcagatatacgtcatggtgttattactgctcctatcttatttgcaatggaagaatttcctcaattaagagaagtagtagatcaagtagaaaaagatccaagaaatgtagacatagctttagaatatttaggtaaaagtaaaggtattcaacgtgctcgtgaattagcaatggaacacgcaaatttagctgctgcagctattggttctttacctgaaacagataacgaagatgttaaacgttcacgtcgtgctttaattgatttaacacacagagtaattacacgtaacaaaggtaccggtgagaatttatactttcaaggtagtggtggaggaggtagtgactataaagatgatgacgataaaggaaccggttaa

SEQ ID NO: 90 - DNA, 1092 bp, Artificial Sequence
  Description of Artificial Sequence: Synthetic polynucleotide
atggtaccagtagtttctgaacgtttaagacattctgtaacaactggtattccagcattaaaaacagcagctgaatatttctttcgtcgtggtatcgaaggaaaacgtttaagacctacattagcattattaatgagtagtgctttatcaccagctgctccatcaccagagtatttacaagttgatacaagacctgctgcagaacaccctcatgaaatgcgtcgtcgtcaacaacgtttagctgaaattgcagaattaatccatgtagcttcattacttcacgatgatgttattgatgacgcacaaacacgtcgtggtgttttaagtttaaatacatctgttggtaataaaacagctatcttagcaggtgatttcttattagctcgtgcatctgtaacattagctagtttaagaaactctgaaattgtagaattaatgtcacaggttttagaacacttagtatctggtgaaattatgcaaatgactgctacttcagaacaacttttagatttagaacattatttagcaaaaacatattgtaaaactgcttcattaatggctaatagttctcgttctgttgcagttcttgcaggtgcagctcctgaagtttgtgatatggcatggtcatacggtcgtcatttaggtattgctttccaagtagttgacgatttattagatttaacaggttcatcttctgttttaggtaaacctgctttaaacgatatgcgttctggtttagcaacagcaccagtattattcgctgcacaagaagaacctgcattacaggctcttatattacgtcgttttaaacacgacggtgacgtaacaaaagcaatgtcattaattgaacgtacacaaggcttacgtcgtgctgaagaacttgcagcacaacacgcaaaagctgctgctgatatgattcgttgcttacctacagctcaatcagaccatgcagaaattgctcgtgaagcattaattcaaattacacatcgtgttttaacacgtaaaaaaggtaccggtgaaaacttatactttcaaggttctggtggtggtggatcagattataaagatgatgatgacaaaggaaccggttaa

SEQ ID NO: 91 - DNA, 987 bp, Artificial Sequence
  Description of Artificial Sequence: Synthetic polynucleotide
atggtaccagattttccacaacaattagaagcatgtgttaaacaagcaaatcaagcattatcacgtttcatcgcaccacttccattccaaaatactcctgttgttgaaacaatgcaatatggtgcattattaggaggtaaaagattaagaccatttcttgtatatgcaacaggtcacatgtttggagtatctactaacacattagatgctccagctgctgcagttgaatgtattcatgcatatagtttaattcatgatgatttacctgcaatggatgatgatgacttaagaagaggtttacctacatgtcatgttaaatttggtgaagctaatgctattttagctggcgatgcacttcaaactcttgcattcagtattttatcagatgctgatatgccagaagtttcagatcgtgatcgtatttctatgatatctgaattagcttctgctagtggtattgctggtatgtgcggtggccaagctcttgatttagacgcagaaggaaaacacgttcctttagatgctttagagcgtatacatcgtcacaaaacaggagctttaattagagctgctgttcgtcttggtgctttatcagctggagacaaaggtcgtcgtgctttaccagttttagacaaatacgctgaaagtattggtttagcttttcaagttcaggatgatatcttagatgttgtaggtgatactgctactttaggtaaacgtcaaggtgctgatcaacagttaggcaaatctacatacccagcacttttaggtttagaacaagctcgtaaaaaagcaagagacttaattgacgatgctcgtcaaagtcttaaacaattagcagaacaatcacttgatacaagtgctttagaagcattagcagattacattattcaacgtaataaaggtaccggtgaaaatttatattttcaaggttctggtggtggaggttcagactataaagatgacgatgataaaggaaccggttaa

SEQ ID NO: 92 - DNA, 1242 bp, Artificial Sequence
  Description of Artificial Sequence: Synthetic polynucleotide
atggtaccaagtgttagttgttgttgtagaaatttaggaaaaactatcaaaaaagctattccaagtcaccacttacatttacgttctttaggtggtagtttatatagaagacgtattcaatcatcttcaatggaaacagacttaaaatctacattcttaaatgtttattcagttcttaaatcagatttattacacgacccatcatttgaatttacaaatgaaagtcgtttatgggtagatagaatgcttgattataatgttcgtggcggtaaacttaatcgtggtctttctgtagtagactctttcaaattacttaaacaaggtaatgatttaactgaacaagaagttttcttatcttgtgcattaggttggtgtattgagtggttacaggcttactttttagttcttgatgatattatggataattcagttacacgtcgtggtcaaccttgttggtttcgtgtaccacaagttggtatggtagctattaatgatggcattcttcttcgtaaccatattcatcgtattcttaaaaaacacttccgtgataaaccatattatgtagatttagttgaccttttcaatgaagtagagttacaaactgcatgtggacaaatgattgatttaatcacaacatttgaaggtgaaaaagacttagctaaatatagtttatcaattcaccgtcgtattgttcaatacaaaactgcatattactcattctatttaccagttgcatgtgctcttttaatggctggcgaaaatttagaaaaccacattgatgttaaaaatgtattagtagatatgggtatttactttcaagttcaggatgattatttagactgttttgctgatcctgaaacattaggtaaaattggcactgatattgaggactttaaatgttcttggttagttgtaaaagcattagaacgttgtagtgaagaacaaacaaaaattctttacgaaaactatggcaaacctgatccatctaatgttgctaaagtaaaagatttatacaaagaattagatttagaaggcgttttcatggaatatgaatctaaatcatacgagaaattaactggtgctatcgaaggtcaccaatctaaagcaattcaagctgttcttaaatctttcttagcaaaaatctataaacgtcaaaaaggtaccggtgaaaacttatactttcaaggtagtggtggcggtggtagtgattataaagatgatgatgataaaggaaccggttaa

SEQ ID NO: 93 - DNA, 1116 bp, Artificial Sequence
  Description of Artificial Sequence: Synthetic polynucleotide
atggtaccagctgatcttaaatcaacattcttagatgtttattcagtattaaaaagtgatttattacaagatccatcttttgaatttacacacgaaagtcgtcaatggttagaacgtatgttagattataatgttcgtggaggcaaattaaacagaggtttaagtgtagtagacagttacaaacttttaaaacaaggtcaagacttaacagaaaaagaaacatttttatcttgtgctttaggttggtgtattgaatggttacaagcatacttcttagttttagacgatattatggataattctgtaactagacgtggtcaaccatgttggtttcgtaaaccaaaagtaggtatgattgctattaatgatggaatacttcttcgtaaccacattcatcgtattcttaaaaaacactttcgtgaaatgccttattatgtagaccttgtagacttatttaacgaagtagaatttcaaacagcttgtggtcaaatgattgacttaattacaacatttgatggtgaaaaagacctttcaaaatattcacttcagattcaccgtcgtattgttgagtacaaaacagcatactactctttctatttacctgtagcatgtgctttacttatggcaggtgaaaatttagaaaatcacacagatgttaaaactgtattagttgatatgggtatctatttccaagttcaagatgattatttagattgcttcgctgatccagaaacattaggtaaaattggtacagatattgaagactttaaatgtagttggttagtagtaaaagcattagaacgttgtagtgaagaacaaacaaaaattctttacgaaaattatggaaaagctgaaccttcaaatgtagctaaagttaaagcattatacaaagaattagatttagagggtgcatttatggaatatgaaaaagaatcatacgagaaacttacaaaacttattgaagcacatcaatcaaaagctattcaagcagttcttaaatctttcttagctaaaatttataaacgtcaaaaaggtaccggtgaaaacttatactttcaaggctctggaggtggtggttcagactataaagatgatgatgataaaggaaccggttaa

SEQ ID NO: 94 - DNA, 1170 bp, Artificial Sequence
  Description of Artificial Sequence: Synthetic polynucleotide
atggtaccaagtggcgaacctactccaaaaaaaatgaaagcaacatacgttcacgaccgtgaaaactttacaaaagtatacgaaactcttcgtgacgaattacttaacgatgattgtcttagtccagctggttcacctcaggctcaagctgctcaagagtggtttaaagaagttaatgattataatgttcctggtggaaaacttaaccgtggtatggctgtatatgacgttttagcttcagttaaaggtccagatggtttaagtgaagacgaagtatttaaagctaacgctcttggttggtgtattgagtggttacaagcatttttcttagttgctgatgatataatggatggttcaattacacgtcgtggccaaccttgttggtacaaacaacctaaagttggtatgattgcttgtaatgattacatcttattagaatgctgtatttactcaattcttaaaagacattttagaggtcacgctgcatacgctcaacttatggaccttttccatgaaactacattccagacttcacacggtcaattattagatttaacaacagcacctatcggttctgtagacttatcaaaatatacagaagataattaccttcgtattgtaacatataaaactgcatactattctttttatttacctgtagcatgtggtatggtattagctggcattacagatccagctgcttttgatcttgcaaaaaatatttgtgttgaaatgggtcaatatttccagattcaagacgattatttagattgctatggtgaccctgaggttattggtaaaatcggtacagacatagaagacaacaaatgtagttggttagtttgcacagctcttaaaatcgcaacagaagaacaaaaagaggttataaaagctaattatggtcacaaagaggctgaatcagtagcagcaattaaagcattatacgttgaattaggtattgaacaacgttttaaagactatgaagctgcatcatacgcaaaattagaaggtacaattagtgaacaaactttattacctaaagcagtatttacttctttattagctaaaatctataaaagaaaaaaaggtaccggtgagaacttatactttcaaggtagtggaggtggtggttcagactataaagatgatgatgataaaggaaccggttaa

SEQ ID NO: 95 - DNA, 636 bp, Artificial Sequence
  Description of Artificial Sequence: Synthetic polynucleotide
atggtaccacaaactgaacatgttatcttattaaacgctcaaggtgttcctacaggtacattagaaaaatatgctgcacacactgctgatactcgtttacacttagctttctcatcttggttattcaatgctaaaggtcaacttttagttacaagacgtgcattaagtaaaaaagcatggcctggtgtttggactaactcagtttgtggtcatccacaattaggtgaaagtaatgaagatgcagttatacgtcgttgcagatatgaattaggtgttgaaataactccaccagaatcaatttatccagatttccgttatcgtgcaactgatcctagtggtatcgttgaaaacgaagtatgtcctgtttttgctgcacgtacaacaagtgcattacaaattaatgatgatgaagtaatggattatcaatggtgtgacttagctgatgttttacatggtattgatgcaacaccatgggcattttcaccatggatggtaatgcaagcaacaaatcgtgaagcacgtaaaagattaagtgcttttacacagttaaaaggtaccggtgaaaacttatactttcaaggtagtggaggtggtggttctgactataaagatgacgatgataaaggaaccggttaa

SEQ ID NO: 96 - DNA, 969 bp, Artificial Sequence
  Description of Artificial Sequence: Synthetic polynucleotide
atggtaccacttcgtagtttattaagaggtttaacacacattcctcgtgttaatagtgctcagcaaccttcttgcgctcacgctcgtcttcaatttaaacttcgttctatgcaattattagcagaaaaccgtacagatcacatgcgtggtgcttctacatgggcaggtggtcagtctcaagatgaattaatgcttaaagatgaatgtatcttagtagatgctgatgataacattactggtcacgcttctaaattagaatgtcacaaatttcttccacatcaaccagctggattattacaccgtgctttttctgtatttcttttcgacgatcaaggtcgtttacttttacaacaacgtgctcgtagtaaaattacatttccatctgtatgggctaatacatgttgtagtcatccattacatggtcaaacaccagatgaagtagatcaacaatcacaagtagcagacggaactgtaccaggtgcaaaagctgctgcaatcagaaaattagaacatgaattaggtattccagctcaccaattaccagcatcagcttttcgtttcttaacacgtcttcactattgtgcagctgacgttcaacctgcagcaacacaatctgcattatggggtgaacacgaaatggattacattttattcattagagctaatgttacacttgctcctaatcctgacgaagtagatgaggtacgttatgtaactcaagaagaattaagacaaatgatgcaaccagataatggtttacaatggtcaccatggttccgtattattgcagcaagatttttagaacgttggtgggctgatttagatgctgcattaaatacagataaacatgaagactggggaacagttcatcacattaacgaagctggtaccggtgaaaacttatactttcaaggatcaggaggcggtggaagtgattataaagatgatgatgataaaggaaccggttaa

SEQ ID NO: 97 - DNA, 1725 bp, Artificial Sequence
  Description of Artificial Sequence: Synthetic polynucleotide
atggtaccaagaagatcaggcaattataacccaacagcatgggacttcaattatatccaatcattagacaatcaatacaaaaaagaacgttactctactcgtcacgctgaattaacagttcaagttaaaaaattattagaagaagaaatggaagctgttcaaaaacttgaacttatagaggatcttaaaaacttaggcatttcttacccatttaaagataatattcaacaaatcttaaatcaaatttacaatgaacacaaatgttgtcacaactcagaagttgaagaaaaagacctttatttcactgctttacgttttagattattacgtcaacaaggttttgaagtaagtcaagaagtatttgatcactttaaaaacgaaaaaggtacagattttaaacctaatttagcagatgatactaaaggattattacaattatatgaagcatcattcttattacgtgaagcagaagacacattagaacttgctcgtcaattctctactaaacttttacaaaaaaaagttgatgaaaacggtgacgataaaattgaagataacttattactttggattagacgtagtttagaattaccattacattggcgtgtacaaagattagaagctcgtggctttttagatgcttacgttcgtagacctgatatgaatcctattgtatttgaattagcaaaattagactttaacattactcaagcaacacaacaagaagaacttaaagatttatcaagatggtggaatagtactggcttagctgaaaaacttccttttgctcgtgatcgtgtagttgaatcatatttctgggctatgggtacttttgaaccacatcaatacggataccaacgtgaattagttgctaaaatcattgcacttgctacagttgtagacgatgtttacgatgtatatggtactttagaggaattagaactttttactgatgctattcgtcgttgggaccgtgaatctattgaccaattaccatattacatgcaattatgttttcttactgtaaacaactttgtttttgagttagctcacgacgtattaaaagataaatcattcaattgtttacctcatttacaaagatcatggttagatttagctgaagcataccttgtagaagcaaaatggtatcatagtcgttatacaccttctttagaagaatatcttaatattgctcgtgtttcagtaacatgtccaactattgtttctcaaatgtattttgcattaccaattccaatcgaaaaacctgtaattgagatcatgtacaaatatcacgatatcttatacttatcaggtatgttattacgtttaccagatgacttaggaacagcatcattcgaacttaaacgtggtgatgtacaaaaagcagttcaatgttatatgaaagaacgtaatgttcctgaaaatgaagctcgtgaacatgttaaattcttaattcgtgaggcttctaaacaaattaatacagcaatggcaacagactgtccatttacagaagattttgcagttgcagcagcaaacttaggtcgtgtagcaaattttgtatatgttgatggtgatggttttggagtacaacacagtaaaatctatgagcaaattggtacacttatgtttgaaccatatccaggtaccggtgaaaacttatactttcaaggtagtggtggtggaggttctgattacaaagacgatgatgataaaggaaccggttaa

SEQ ID NO: 98 - DNA, 1722 bp, Artificial Sequence
  Description of Artificial Sequence: Synthetic polynucleotide
atggtaccaagaagaagtggaaactataaacctacaatgtgggattttcaatttattcaaagtgtaaataatctttacgctggtgataaatacatggaacgtttcgatgaagtaaaaaaagaaatgaaaaaaaacttaatgatgatggttgagggtttaatagaggaattagatgttaaattagaattaatagataatttagaaagattaggtgttagttatcatttcaaaaatgaaataatgcaaatccttaaatctgtacaccagcaaatcacttgtcgtgataattcattatactctactgcattaaaatttcgtttattacgtcaacacggattccacattagtcaagacatctttaacgattttaaagatatgaatggcaatgttaaacaaagtatctgtaacgatactaaaggtttattagaactttatgaagcatctttcttatctactgaatgtgaaacaacacttaaaaacttcactgaagcacacttaaaaaattatgtttatattaaccactcatgtggagatcaatacaataacataatgatggaattagttgttcacgctttagaattaccacgtcactggatgatgcctcgtttagagacacgttggtatatatcaatttatgaacgtatgcctaatgctaatccacttttacttgaacttgctaaattagacttcaatattgttcaagctacacaccaacaagacttaaaatcattatcacgttggtggaaaaacatgtgtttagctgaaaaattatcattttctcgtaaccgtttagtagaaaatcttttctgggcagttggaactaattttgaaccacaacacagttatttccgtcgtttaatcactaaaatcattgtttttgttggtattattgatgatatttatgatgtttacggcaaacttgatgagttagaattattcactttagctgtacaacgttgggatacaaaagcaatggaagacttaccatattacatgcaagtttgttatttagctttaattaatacaacaaatgatgttgcttatgaagttcttcgtaaacataacattaatgtattaccatacttaactaaatcttggacagacttatgtaaatcatatttacaagaagctcgttggtactacaatggttacaaaccttcattagaggaatatatggataatggttggattagtatagcagttcctatggtattagcacatgcacttttcttagttacagatccaattacaaaagaagcattagaatcattaacaaactatcctgatattattcgttgctcagctacaatattccgtttaaatgatgatcttggtacaagttcagatgaattaaaacgtggagatgtaccaaaatcaattcaatgctatatgaacgaaaaaggcgtttcagaggaagaagctcgtgaacatattcgtttcttaatcaaagaaacatggaaattcatgaacactgcacaccataaagagaaaagtttattttgtgagacatttgtagaaattgcaaaaaatattgcaacaacagctcattgtatgtacttaaaaggtgattctcacggtattcaaaacactgatgttaaaaactcaataagtaatatacttttccatccaattattatcggtaccggtgaaaacctttactttcaaggttcaggtggtggcggttcagactataaagatgacgatgataaaggaaccggttaa

SEQ ID NO: 99 - DNA, 1737 bp, Artificial Sequence
  Description of Artificial Sequence: Synthetic polynucleotide
atggtaccaagacgtagtggaaattatgagccatctgcatgggacttcaattacttacaatctcttaataattatcaccataaagaagaacgttacttacgtcgtcaagctgatttaattgaaaaagtaaaaatgattcttaaagaagagaaaatggaagcattacagcaattagaacttatagacgatcttcgtaatttaggtctttcatattgttttgatgatcaaattaatcatattcttacaacaatttacaaccaacattcttgtttccattatcacgaagctgcaacaagtgaagaagctaacttatatttcacagctttaggtttccgtttacttcgtgaacacggattcaaagtatcacaagaagtatttgaccgtttcaaaaatgaaaaaggtacagattttcgtccagatttagtagatgatactcaaggtttattacaactttatgaagcatctttccttcttcgtgaaggtgaagacactttagaatttgcacgtcaatttgctactaaatttcttcaaaaaaaagttgaggagaaaatgatagaagaggaaaatcttttatcttggactttacattcacttgaattaccattacattggcgtatacaacgtttagaagctaaatggtttttagatgcttatgctagtcgtcctgatatgaatccaataatctttgaattagcaaaattagaatttaacattgctcaggcacttcaacaagaagaacttaaagatttatcaagatggtggaacgatactggtattgctgaaaaattacctttcgctcgtgatagaatcgttgaatctcattattgggcaattggtactttagaaccttatcaataccgttatcagcgttcattaattgcaaaaatcattgctttaactacagttgttgatgatgtatatgatgtttacggtacattagacgaattacagttatttactgatgcaattcgtcgttgggacattgaaagtataaatcaattaccttcttatatgcaattatgttatttagctatttataatttcgtatcagaattagcttatgatattttcagagataaaggttttaattctttaccatatttacacaaaagttggcttgacttagttgaggcttactttcaagaagcaaaatggtatcattctggctacacaccatcattagaacaatacttaaatatcgctcaaatttctgtagcaagtccagctatattaagtcaaatttactttactatggctggttcaattgataaaccagtaatcgaatcaatgtacaaatatagacacattttaaacttatctggtatattacttagattaccagatgacttaggtactgctagtgatgaattaggtcgtggtgatttagcaaaagcaatgcaatgttacatgaaagagcgtaacgtttctgaagaagaagctcgtgatcatgtacgtttcttaaatcgtgaggtttcaaaacaaatgaatcctgctcgtgctgctgatgattgtccattcactgatgattttgtagtagctgctgctaatttaggaagagttgcagatttcatgtatgttgaaggcgatggtttaggtttacaatacccagctatccaccaacacatggcagaacttttatttcacccttacgcaggtaccggtgaaaacttatactttcaaggttcaggtggtggaggttctgactataaagatgatgatgataaaggaaccggttaa

SEQ ID NO: 100 - DNA, 1737 bp, Artificial Sequence
  Description of Artificial Sequence: Synthetic polynucleotide
atggtaccaagaagatcaggaaattatcaacctagtgcatgggattttaactatatccaatctcttaataacaaccattctaaagaagaacgtcacttagagcgtaaagcaaaacttattgaagaagtaaaaatgttattagagcaagaaatggctgctgtacaacaattagagcttattgaagaccttaaaaacttaggtttatcttacttattccaagatgaaatcaaaataatccttaattctatttacaatcatcataaatgttttcataataatcacgaacaatgtattcacgttaatagtgacttatactttgttgcattaggcttccgtttatttcgtcaacatggtttcaaagtttctcaagaggtttttgactgttttaaaaacgaagaaggatcagactttagtgctaacttagcagatgatactaaaggtttacttcaattatacgaggcttcatatttagttacagaagatgaagacacattagaaatggcacgtcaattttcaactaaaatcttacaaaaaaaagtagaagagaaaatgattgagaaagagaacttattaagttggactttacatagtttagaattaccacttcactggcgtattcaacgtttagaagcaaaatggttccttgatgcttatgctagtcgtccagatatgaatccaattatttttgaattagctaaattagagtttaacattgctcaagcattacaacaagaagaattaaaagatttaagtagatggtggaatgatacaggcattgctgaaaaattaccttttgctcgtgatagaatagtagagagtcattactgggcaattggtactttagaaccttatcaatatagatatcaacgttcattaattgctaaaattattgctttaacaacagttgttgatgacgtttacgacgtatatggaactttagatgaattacagttatttacagacgctattcgtcgttgggatattgaatctattaatcaattaccaagttatatgcaattatgctatttagctatttataactttgtttctgaattagcatacgatatttttcgtgacaaaggattcaattctttaccttaccttcataaatcatggttagatttagtagaagcatactttgttgaagctaaatggtttcatgatggttatactccaactcttgaagaatatttaaataactcaaaaattactattatatgtcctgctattgttagtgaaatctacttcgcattcgctaattcaattgataaaacagaagttgaatcaatctacaaatatcacgatattttatatttatcaggaatgcttgcacgtttaccagacgacttaggtacttcatcatttgaaatgaaaagaggtgatgttgctaaagctattcaatgttacatgaaagaacataatgcttcagaggaagaagctcgtgaacacattcgtttcttaatgcgtgaagcatggaaacacatgaatactgctgcagctgctgatgactgtccatttgaatctgatttagtagtaggtgctgcatcattaggtagagttgcaaactttgtatatgttgaaggtgacggttttggtgtacaacattcaaaaatacatcaacaaatggctgaattacttttttatccatatcaaggtaccggtgaaaacttatactttcaaggtagtggaggtggtggtagtgactataaagacgatgacgataaaggaaccggttaa

SEQ ID NO: 101 - DNA, 1707 bp, Artificial Sequence
  Description of Artificial Sequence: Synthetic polynucleotide
atggtaccaagaagaagtgctaattatcaagcaagtatttgggatgataatttcattcaaagtcttgcatctccttatgcaggagaaaaatatgcagaaaaagcagaaaaacttaaaacagaagttaaaactatgattgatcaaacaagagatgaacttaaacaattagaacttattgataacttacaacgtttaggtatatgtcatcactttcaagaccttacaaaaaaaattttacaaaaaatttatggagaagaacgtaacggagatcaccaacattacaaagaaaaaggcttacattttacagcattacgtttccgtattttacgtcaggacggttatcatgttccacaagatgtattttcatcatttatgaataaagctggtgactttgaagaatctttaagtaaagacacaaaaggtttagttagtttatatgaggcttcttacttatcaatggaaggtgaaactattttagatatggcaaaagacttttcatctcaccatttacataaaatggttgaagatgctactgacaaacgtgtagctaatcaaattatccattctcttgaaatgccacttcacagacgtgttcaaaaacttgaagcaatttggtttattcaattctacgaatgcggctctgatgctaatccaactttagtagaattagcaaaattagatttcaacatggttcaggcaacataccaagaagaattaaaacgtttatcacgttggtatgaagaaacaggcttacaagagaaactttcattcgctcgtcaccgtcttgctgaagcattcttatggtctatgggtattattccagaaggacactttggttatggtcgtatgcacttaatgaaaattggtgcttacattacattacttgatgatatttatgatgtttatggtactttagaagaacttcaagtattaacagaaattattgaacgttgggatattaacttattagatcaattacctgaatacatgcaaatcttctttttatacatgtttaattctacaaatgaacttgcttatgaaattttacgtgatcaaggtatcaatgtaatatcaaacttaaaaggattatgggtagagttatctcagtgttactttaaagaagctacttggttccataacggttacacaccaacaactgaagaatatcttaatgttgcttgtatttctgctagtggtcctgttattttattttcaggttactttactactactaatcctattaataaacacgaattacaatctttagaacgtcacgcacattcattatctatgatattacgtttagctgatgatttaggtacatcaagtgatgaaatgaaacgtggagatgtaccaaaagctattcaatgttttatgaatgacactggttgttgtgaagaagaagcacgtcaacacgtaaaaagattaatagatgctgaatggaaaaaaatgaacaaagacatcttaatggagaaaccatttaaaaatttttgtccaactgctatgaatttaggtcgtatttctatgagtttttatgaacacggagatggttatggaggtcctcactctgatacaaaaaaaaaaatggtatctttatttgtacaaccaatgaatattactattggtaccggtgaaaacctttattttcaaggttctggtggtggcggttcagattataaagatgatgacgacaaaggaaccggttaa

SEQ ID NO: 102 - DNA, 1716 bp, Artificial Sequence
  Description of Artificial Sequence: Synthetic polynucleotide
atggtaccaagacgttcagctaactatcaacctagtatttggaaccacgattacattgaatcacttcgtatcgaatatgttggtgaaacatgtacacgtcaaattaacgttttaaaagaacaagttcgtatgatgttacacaaagttgttaatccattagaacaattagaattaattgaaattttacaacgtttaggtttaagttaccatttcgaagaagaaataaaacgtattttagatggtgtttacaataacgatcatggtggtgatacatggaaagcagaaaacctttatgcaacagctcttaaattccgtcttttacgtcagcacggttattctgtttctcaagaagttttcaactcttttaaagatgagcgtggcagtttcaaagcatgtttatgtgaagatactaaaggtatgttatcactttatgaagcatctttctttcttattgaaggtgaaaacattttagaggaagctagagactttagtacaaaacatcttgaagaatatgtaaaacaaaataaagagaaaaacttagctactttagttaatcactcattagaatttccattacattggcgtatgcctcgtttagaagctcgttggttcatcaatatctatcgtcataatcaagatgtaaatccaatccttttagaatttgctgaacttgacttcaatattgtacaagctgctcaccaagcagatttaaaacaagtatcaacatggtggaaatcaactggtttagtagaaaatctttcattcgctcgtgatcgtcctgtagaaaacttcttttggacagttggtcttattttccaaccacaattcggttattgtcgtagaatgtttactaaagtattcgcattaattactacaattgatgacgtatatgatgtatatggtactttagatgaattagaacttttcacagacgttgttgaaagatgggatattaatgcaatggatcaattacctgattatatgaaaatttgctttttaacattacacaatagtgttaacgaaatggcattagacactatgaaagaacaacgttttcacatcattaaataccttaaaaaagcatgggttgatctttgtcgttattacttagttgaagctaaatggtatagtaataaatatagaccttctttacaagaatacattgaaaatgcatggatttcaattggtgctccaactattttagttcatgcatatttcttcgttacaaatccaattacaaaagaagcattagactgtttagaagaatatccaaacattattcgttggagtagtattattgcacgtttagctgatgatttaggtacttcaacagacgaattaaaacgtggtgacgtaccaaaagcaattcaatgttatatgaatgaaacaggtgcttcagaagaaggtgctcgtgagtacattaaatacttaatttctgctacttggaaaaaaatgaacaaagatagagcagcatcaagtccattttcacatatcttcattgaaattgctcttaatttagcacgtatggcacaatgtttatatcaacacggtgacggccacggtttaggtaaccgtgaaacaaaagatcgtatactttcattacttattcaaccaattccattaaacaaagatggtaccggtgagaacttatactttcaaggctcaggtggtggtggttctgattacaaagatgatgatgataaaggaaccggttaa

SEQ ID NO: 103 - DNA, 1800 bp, Artificial Sequence
  Description of Artificial Sequence: Synthetic polynucleotide
atggtaccaagaagaattggagactatcactcaaacttatggaatgatgacttcattcaatcattaacaacaccatacggtgctccatcatatattgaacgtgctgatagattaatatctgaagtaaaagaaatgtttaatagaatgtgtatggaagatggtgagttaatgtctccattaaatgatcttattcaaagattatggactgttgatagtgttgaacgtttaggtatagatcgtcacttcaaaaatgaaataaaagctagtttagattatgtatactcatactggaacgaaaaaggtatcggttgtggtcgtcaatcagtagttacagatttaaactctactgctcttggattaagaattttacgtcaacatggttacacagtttcaagtgaagttttaaaagtttttgaagaagaaaacggtcaatttgcttgttcaccttcacagactgagggcgaaattcgttcattcttaaacttatatcgtgcttcattaattgcttttcctggtgaaaaagtaatggaagaagctcaaatcttttctagtcgttacttaaaagaagcagttcagaaaattccagtttcaggtttatctcgtgaaataggcgatgttttagaatatggttggcacacaaacttacctcgttgggaagctcgtaactatatggacgtattcggtcaagacacaaatacatcattcaacaaaaacaaaatgcaatatatgaatacagagaaaattcttcaattagtaaaattagagtttaatatctttcattcattacaacaacgtgaattacaatgtttattacgttggtggaaagaaagtggtcttccacaattaacatttgcacgtcaccgtcacgttgaattttacactttagcttcttgtattgcatgtgaaccaaaacacagtgcatttcgtttaggttttgcaaaaatgtgtcacttagtaacagttttagatgatgtatatgacacatttggcaaaatggatgaattagaactttttactgcagctgttaaacgttgggacttatcagaaactgagcgtttacctgagtatatgaaaggtttatatgttgtagttttcgagactgttaatgaattagcacaagaagcagagaaaactcaaggacgtaatacattaaattacgttcgtaaagcatgggaagcatacttcgatagttatatgaaagaagcagaatggatctcaacaggctatttaccaacattcgaagagtattgtgaaaacggtaaagtatcaagtgcatatagagttgctgcacttcaacctattttaacattagatgtacaacttccagatgacatcttaaaaggtattgattttccatctcgtttcaatgatttagcatcttcatttcttcgtttacgtggagatactagatgttacgaggctgatcgtgctcgtggtgaagaagcaagttgtatttcttgttacatgaaagacaatccaggttcaactgaagaagatgcattaaatcacattaatgctatgataaatgatattattcgtgaattaaactgggaatttcttaaaccagactcaaatatcccaatgccagctcgtaaacatgctttcgatattacaagagctttacatcacttatatatttatcgtgacggtttttctgttgctaacaaagagactaaaaatcttgttgagaaaactttattagaatcaatgttattcggtaccggtgagaacctttattttcaaggttcaggtggtggtggttcagattataaagacgatgatgataaaggaaccggttaa

SEQ ID NO: 104 - DNA, 1731 bp, Artificial Sequence
  Description of Artificial Sequence: Synthetic polynucleotide
atggtaccaagaagatcagctaattatcaacctagtcgttgggatcatcatcaccttttaagtgtagaaaacaaattcgctaaagataaacgtgtaagagaacgtgacttacttaaagaaaaagttcgtaaaatgttaaatgacgaacagaaaacttacttagatcaattagaatttattgacgatcttcaaaaattaggtgttagttatcacttcgaagcagaaatagataatatacttacaagttcatacaaaaaagatcgtacaaatatacaagaaagtgatttacacgcaactgcattagagtttcgtctttttcgtcaacacggttttaacgtttcagaagatgtatttgatgtatttatggaaaattgtggtaaattcgaccgtgatgacatttatggtttaatttcattatatgaagctagttatctttctactaaacttgacaaaaatcttcaaatctttatccgtccatttgctactcaacaattacgtgattttgtagatactcacagtaatgaagatttcggttcatgtgatatggtagaaatagttgttcaagcattagacatgccatactattggcaaatgcgtcgtttatctacacgttggtatattgatgtttatggtaaaagacaaaattacaaaaacttagtagttgttgaatttgcaaaaattgatttcaatattgttcaagctattcaccaggaagaacttaaaaatgtatcatcttggtggatggaaactggtttaggtaaacaactttattttgctcgtgatcgtattgtagagaactatttttggacaattggtcaaattcaagaacctcaatatggatatgttagacaaacaatgactaaaatcaatgctttattaacaacaattgatgatatttatgatatatacggtacattagaagaattacagttattcacagttgcatttgagaattgggacataaatcgtttagacgaattaccagaatatatgcgtttatgtttcttagttatctataacgaagtaaatagtatagcatgtgaaattcttagaacaaaaaatattaacgttattcctttcttaaaaaaatcttggactgatgtaagtaaagcatacttagttgaagctaaatggtataaatcaggccataaaccaaatttagaagagtatatgcaaaatgcacgtatttctatttcttcaccaacaatctttgttcacttttattgtgtattttcagaccaattatctattcaagttttagaaactttatcacaacaccaacaaaatgttgtaagatgtagttcttctgttttccgtttagctaatgacttagtaacttctccagatgaattagctagaggtgatgtttgtaaatcaattcaatgttatatgtcagaaactggtgcaagtgaagataaagctagatcacacgttcgtcaaatgattaatgatttatgggacgaaatgaattacgagaaaatggcacattcaagtagtatcttacatcatgattttatggagacagtaatcaatttagctagaatgtctcaatgtatgtaccaatatggtgacggacacggttctccagaaaaagctaaaattgtagatcgtgtaatgagtttacttttcaaccctattcctttagatggtaccggtgagaatttatattttcaaggctctggaggtggtggttcagattataaagatgatgacgacaaaggaaccggttaa

SEQ ID NO: 105 - DNA, 1713 bp, Artificial Sequence
  Description of Artificial Sequence: Synthetic polynucleotide
atggtaccaagaagaagtgcaaactatcaaccttcattatggcaacatgaatacttattatcattaggcaacacttatgttaaagaagataatgttgaaagagtaactcttttaaaacaagaagtttctaaaatgttaaacgaaacagaaggtttacttgaacaacttgaattaattgacactttacaaagattaggtgtttcttatcattttgaacaggagattaaaaaaacattaactaatgttcatgttaaaaacgtacgtgctcataaaaatcgtattgatcgtaaccgttggggcgatttatatgcaactgcattagaatttcgtttattacgtcaacatggtttttctattgctcaagacgtttttgatggtaatattggtgttgacttagacgacaaagacattaaaggtattttaagtttatacgaagctagttacttatcaacacgtattgatacaaaacttaaagaatcaatctattacacaacaaaacgtttaagaaaattcgtagaggtaaacaaaaacgaaactaaaagttacactcttcgtcgtatggttattcacgcacttgagatgccttatcaccgtcgtgttggtcgtcttgaagctcgttggtatatcgaggtatatggagaaagacacgacatgaatcctattttattagaattagctaaattagattttaactttgttcaggctatccaccaagacgaattaaaatcattatctagttggtggtctaaaacaggattaacaaaacatttagactttgttcgtgatcgtattacagagggttacttcagtagtgtaggtgttatgtatgaaccagaatttgcatatcatcgtcaaatgcttacaaaagtatttatgcttattacaactattgatgacatctatgacatttacggtacacttgaagaattacaattattcacaactatcgttgaaaaatgggatgttaatcgtttagaagaacttcctaactatatgaaattatgcttcttatgtttagttaacgaaataaatcaaattggatattttgtattaagagataaaggttttaatgtaattccttatcttaaagagtcttgggctgacatgtgtactacatttcttaaagaagctaaatggtacaaatcaggttataaaccaaattttgaagagtatatgcaaaatggctggatttcatcatcagttccaactattcttttacacttattttgtttattaagtgaccaaactttagacattcttggttcttataatcacagtgttgttcgtagttcagcaacaattttacgtcttgcaaatgatttagctacttcttcagaagaattagcaagaggagatacaatgaaatcagttcaatgtcacatgcatgaaactggtgcttcagaagctgaatcaagagcttacattcaaggtattattggcgtagcttgggatgaccttaatatggagaaaaaatcatgtcgtttacaccagggattcttagaagcagcagcaaatttaggacgtgtagcacaatgcgtatatcaatatggagacggtcacggttgtccagataaagcaaaaacagtaaatcatgttcgtagtttattagttcacccattaccattaaacggtaccggtgaaaacctttattttcaaggtagtggtggaggtggttctgattataaagacgacgatgacaaaggaaccggttaa

SEQ ID NO: 106 - DNA, 1752 bp, Artificial Sequence
  Description of Artificial Sequence: Synthetic polynucleotide
atggtaccagcttctccacctgctcatcgttcatctaaagcagcagacgaagagttaccaaaagcatcttctacattccatccatctctttggggttcatttttcttaacatatcagccacctacagctccacaacgtgcaaatatgaaagaacgtgctgaagttcttcgtgaacgtgttcgtaaagtattaaaaggttcaacaacagatcaattacctgaaacagttaacttaattcttacattacaaagacttggtttaggttattactatgaaaatgaaattgacaaattacttcatcaaatttactctaattcagattataacgtaaaagacttaaacttagtttctcaacgtttttacttacttcgtaaaaacggttatgacgtaccttctgatgttttcttatcttttaaaactgaagaaggtggtttcgcttgtgctgcagctgacacacgttcacttttaagtttatacaatgctgcttaccttcgtaaacatggtgaagaagtattagatgaagcaatttcatcaacacgtttaagattacaagacttattaggtcgtttattacctgaatcaccattcgctaaagaagtatcaagttcacttcgtacacctttattccgtcgtgtaggtattttagaagctcgtaactatattccaatctatgaaactgaagctacaagaaatgaagctgtattagagcttgctaaacttaacttcaatttacaacagcttgatttctgtgaagaattaaaacattgtagtgcatggtggaatgagatgattgctaaaagtaaattaacttttgtacgtgaccgtatagttgaagaatacttttggatgaatggtgcatgttatgatccaccatattcattaagtcgtattattcttacaaaaatcactggtttaattactattattgatgatatgttcgatactcatggtacaacagaggattgcatgaaattcgcagaagcatttggtcgttgggatgaatcagcaattcatcttcttccagaatacatgaaagatttttacattttaatgttagaaactttccagtcatttgaagatgcacttggtccagaaaaatcataccgtgtattatacttaaaacaagcaatggaacgtttagtagagttatattctaaagaaatcaaatggcgtgatgacgattatgttccaacaatgtcagaacatttacaagttagtgctgaaacaattgctacaattgctttaacttgctctgcttatgctggtatgggtgatatgtctattcgtaaagaaacatttgaatgggcattatctttccctcaattcattagaacttttggttcatttgtacgtttatcaaatgatgttgtatcaacaaaacgtgaacaaactaaagatcattcaccttcaacagttcactgttatatgaaagaacacggtacaactatggacgatgcttgtgaaaaaatcaaagaattaattgaggactcatggaaagacatgttagaacaatctttagctcttaaaggcttacctaaagtagtacctcaattagtttttgatttctctcgtactacagataacatgtatcgtgaccgtgatgctttaacatcatcagaagcattaaaagaaatgatacagttattattcgtagaacctatacctgaaggtaccggtgagaatctttattttcaaggatcaggtggtggaggctcagattacaaagatgacgacgataaaggaaccggttaa

SEQ ID NO: 107 - DNA, 1761 bp, Artificial Sequence
  Description of Artificial Sequence: Synthetic polynucleotide
atggtaccagaggctttaggaaattttgattatgagagttatactaattttacaaaattaccatcatcacaatggggtgatcaattccttaaattttctatagcagattctgacttcgatgtattagaaagagaaatagaagtattaaaaccaaaagtaagagagaacatttttgtttcatcaagtactgataaagatgcaatgaaaaaaacaattttaagtattcatttcttagatagtttaggtttatcttatcacttcgaaaaagaaatagaggagagtttaaaacatgctttcgagaaaattgaagaccttattgctgatgaaaataaacttcatacaataagtacaattttccgtgtattccgtacatacggctattatatgtcttctgatgtattcaaaattttcaaaggagacgatggtaaattcaaagaaagtttaattgaagacgttaaaggtatgctttctttttatgaagctgttcattttggaacaactactgatcacattttagacgaagctcttagttttacattaaaccacttagagtcacttgcaacaggccgtcgtgcatcaccaccacatattagtaaattaatccaaaatgctttacatattcctcaacatcgtaacatccaggcattagtagctcgtgaatacattagtttttacgaacacgaagaagatcacgatgaaacattattaaaattagctaaattaaactttaaattcttacaacttcactattttcaagaattaaaaacaattacaatgtggtggactaaattagatcatacatctaatttaccaccaaattttcgtgaacgtacagttgaaacatggtttgcagctttaatgatgtatttcgaaccacaatttagtttaggtcgtattatgagtgcaaaattatatttagtaattactttcttagatgacgcatgtgatacatacggatcaatatctgaagtagagtcattagctgattgtttagaacgttgggacccagattatatggaaaatttacaaggtcacatgaaaacagcattcaaattcgttatgtatttattcaaagaatacgaagaaattttacgttcacaaggccgttcattcgtattagagaaaatgattgaggagtttaaaattatcgcacgtaaaaacttagaacttgtaaaatgggctcgtggtggtcacgttccttcttttgacgaatatatagagagtggtggtgctgagattggtacttatgctacaatcgcttgttcaattatgggtcttggtgaaattggtaaaaaagaagcatttgagtggttaatctctcgtcctaaacttgttcgtattttaggtgctaaaacacgtttaatggatgatatcgcagactttgaagaagacatggaaaaaggctatacagctaatgcacttaactattatatgaatgaacacggagtaactaaagaagaagctagtcgtgaacttgagaaaatgaatggtgatatgaacaaaattgtaaacgaagaatgtcttaaaattacaactatgccacgtcgtatcttaatgcaaagtgttaactacgctcgtagtttagatgtattatacacagctgatgatgtatataaccaccgtgaaggcaaacttaaagaatatatgagattacttttagtagatccaattttacttggtaccggtgaaaatctttattttcaaggttcaggtggtggtggttctgattataaagatgatgacgataaaggaaccggttaa

SEQ ID NO: 108 - DNA, 1662 bp, Artificial Sequence
  Description of Artificial Sequence: Synthetic polynucleotide
atggtaccagagagtcaaacaacattcaaatacgaatcattagcatttacaaaacttagtcactgtcaatggacagactattttcttagtgttccaattgatgaaagtgaattagatgttattactcgtgaaattgatattcttaaaccagaagttatggagttattaagtagtcaaggagatgatgaaacaagtaaaagaaaagttcttcttattcagttattactttctttaggtttagcattccactttgaaaatgagattaaaaacatacttgaacacgcatttcgtaaaatagatgatataactggtgacgaaaaagacttatcaacaattagtattatgttccgtgttttccgtacttatggacacaatcttccaagtagtgtttttaaacgtttcacaggtgatgatggtaaatttcagcaaagtttaacagaagacgcaaaaggtattttaagtttatatgaagctgcacatttaggtactactacagattacattttagatgaagctcttaaattcacatctagtcacttaaaaagtttacttgctggtggtacatgtcgtcctcacatcttacgtttaatccgtaatacattatacttaccacaacgttggaacatggaagctgttatcgctcgtgaatacatatcattttacgagcaggaagaagatcacgataaaatgcttttacgtcttgcaaaacttaactttaaacttcttcaattacactacattaaagagcttaaaagtttcattaaatggtggatggaacttggtttaacttctaaatggccttctcaatttcgtgaacgtattgttgaagcatggttagctggattaatgatgtattttgaaccacagttctcaggtggtcgtgttattgctgcaaaattcaactatttacttacaatattagacgacgcatgtgaccactatttttctattcacgaattaacacgtttagttgcatgtgtagaacgttggtcaccagatggtattgacacattagaagatatttcacgttctgtattcaaattaatgttagatgttttcgacgatattggtaaaggtgtacgttcagaaggttctagttaccacttaaaagaaatgttagaggaattaaacactttagttcgtgctaatttagatttagttaaatgggctcgtggaatacaaacagctggtaaagaggcttatgaatgggttcgttcacgtccacgtttaatcaaatctttagcagctaaaggtagacttatggatgatattacagactttgactcagatatgagtaatggattcgcagctaatgctattaactactatatgaaacaatttgttgttacaaaagaagaagctattcttgaatgtcaacgtatgattgtagacattaacaaaactattaatgaagagttattaaaaactacttcagttccaggtcgtgtattaaaacaagctcttaactttggccgtttattagaattattatatacaaaatctgacgatatttacaattgttctgaaggcaaacttaaagaatacattgtaactcttttaattgatcctataagacttggtaccggtgaaaacttatactttcaaggttcaggcggtggtggtagtgattacaaagatgatgatgacaaaggaaccggttaa

SEQ ID NO: 109 - DNA, 1503 bp, Artificial Sequence
  Description of Artificial Sequence: Synthetic polynucleotide
atggtaccagagagtcaaacaaaattcgactacgaatcattagcttttacaaaattatcacattcacaatggactgattactttttatcagtacctatagacgactctgaacttgacgcaattactcgtgaaatcgacattatcaaacctgaagttcgtaaattactttcaagtaaaggtgatgatgaaacttctaaacgtaaagtattacttatccaaagtttattatcattaggtttagcatttcattttgaaaacgaaattaaagatattttagaagatgcatttagacgtattgatgacattacaggtgatgaaaacgacttaagtactattagtattatgttccgtgtattccgtacatacggtcacaatttaccaagtagtgtttttaaacgtttcactggtgatgacggtaaatttgaacgttctttaactgaagatgctaaaggaattttatcattatatgaagctgcacatttaggaacaactactgattatattcttgatgaagcattagaatttacttcatcacacttaaaatctttacttgttggtggtatgtgtcgtccacatattttacgtcttattagaaatactttatatcttccacaacgttggaatatggaagcagtaattgcaagagaatacattagtttttatgaacaagaagaagatcacgataaaatgttacttcgtttagctaaattaaatttcaaattacttcaattacactacattaaagagttaaaaacattcattaaatggtggatggaattaggacttacatcaaaatggccttctcaatttcgtgaacgtattgttgaagcatggttagctggtcttatgatgtattttgaaccacagttttctggaggtcgtgtaatagctgctaaattcaattacttattaacaattttagatgatgcatgtgatcactatttctcaattccagaattaactcgtttagttgattgcgtagaaagatggaatcatgatggtatacatactttagaagacatctcacgtatcatctttaaacttgcattagatgtatttgatgatattggtcgtggtgttcgttctaaaggttgttcttattacttaaaagaaatgttagaagagttaaaaatcttagttcgtgcaaacttagatttagttaaatgggctcgtggtaatcaattacctagttttgaagaacacgttgaggtaggtggtattgctcttacaacatacgcaactttaatgtactcttttgttggcatgggtgaagcagtaggtaaagaagcatacgaatgggtacgttctcgtccacgtttaatcaaaagtttagcagcaaaaggtcgtcttatggacgatattactgatttcgaagtaaaaattatcaacttatttttcgaccttcttttatttgtattcggtaccggtgaaaacttatatttccagggtagtggtggaggaggttcagactacaaagatgacgatgacaaaggaaccggttaa

SEQ ID NO: 110 - DNA, 1725 bp, Artificial Sequence
  Description of Artificial Sequence: Synthetic polynucleotide
atggtaccagcagctttcacagcaaatgcagttgacatgcgtccaccagttattacaattcacccacgttcaaaagatattttctctcaattttctttagatgataaattacaaaaacaatacgctcaaggaatcgaagctcttaaagaagaagctcgttctatgcttatggctgcaaaatctgctaaagtaatgatcttaattgatacacttgaacgtttaggattaggttatcactttgaaaaagaaattgaagagaaattagaagctatttacaaaaaagaggatggtgacgattatgatctttttacaactgctttaagattccgtttacttagacaacaccaacgtcgtgtaccatgttctgtttttgacaaatttatgaataaagagggtaaattcgaagaagaaccattaatttcagatgttgaaggtcttctttcattatatgacgctgcttatttacagattcacggtgaacacattttacaagaggctttaattttcactacacatcatttaactcgtattgaaccacaattagatgatcactctcctttaaaattaaaattaaaccgtgctttagaatttcctttttacagagaaatccctataatctatgcacatttttacatttcagtatatgaacgtgacgattctcgtgatgaagtattattaaaaatggctaaattatcttataatttcttacaaaacttatacaaaaaagaattaagtcaactttctcgttggtggaacaaattagaacttattcctaatttaccttatattcgtgattctgtagctggagcttatttatgggctgttgctttatatttcgaacctcaatattcagacgttcgtatggcaattgctaaacttatccaaattgcagcagctgtagatgatacttacgataattatgctactatacgtgaagctcaattattaacagaagcattagaacgtttaaatgtacacgaaattgacacattaccagattatatgaaaattgtttatcgttttgtaatgtcatggagtgaagatttcgaacgtgatgctacaattaaagaacagatgttagctacaccttatttcaaagctgaaatgaaaaaacttggtcgtgcttataatcaagaacttaaatgggttatggaacgtcaattacctagtttcgaagaatacatgaaaaactctgaaatcacttctggtgtttacattatgtttactgtaattagtccttacttaaatagtgcaacacaaaaaaacattgactggttattatcacaacctcgtttagcatcttcaactgcaattgttatgcgttgttgtaatgatttaggctctaatcaacgtgaatctaaaggaggagaagttatgacatctttagattgctatatgaaacaacacggtgctagtaaacaagaaacaatttctaaattcaaacttattatcgaagatgaatggaaaaacttaaatgaagaatgggctgcaacaacatgtcttccaaaagttatggtagaaatttttcgtaactatgcacgtattgcaggcttttgctacaaaaataacggtgatgcttatacatctccaaaaattgtacaacaatgttttgacgctttatttgtaaatccattaagaattggtaccggtgagaatttatactttcaaggctcaggtggaggtggtagtgattataaagatgatgatgataaaggaaccggttaa

SEQ ID NO: 111 - DNA, 1818 bp, Artificial Sequence
  Description of Artificial Sequence: Synthetic polynucleotide
atggtaccagaatttagagttcatttacaggctgataatgaacagaaaatattccagaaccaaatgaaacctgaacctgaagcatcatatcttattaatcaacgtagatcagctaattacaaacctaatatttggaaaaatgactttttagatcaaagtttaattagtaaatacgacggtgatgaatatcgtaaattaagtgagaaattaatcgaggaagtaaaaatttatatatctgctgagacaatggacttagtagctaaattagaacttattgattctgttcgtaaattaggtttagctaatctttttgaaaaagaaattaaagaagcattagattctatcgcagctattgagtcagataatttaggtactcgtgatgacttatatggtactgctttacactttaaaattttacgtcaacatggttataaagtttctcaagatatttttggtcgtttcatggatgaaaaaggtacattagaaaatcatcacttcgctcacttaaaaggtatgttagaattatttgaagcatctaatttaggttttgaaggtgaagatattttagatgaagcaaaagcatcacttacattagctcttcgtgatagtggtcatatttgttatccagattctaacttaagtcgtgatgtagtacactcattagaattacctagtcaccgtcgtgttcaatggtttgatgttaaatggcaaattaatgcttatgaaaaagatatttgtagagttaatgcaactcttttagaattagcaaaattaaattttaacgtagtacaagcacaacttcaaaaaaacttacgtgaagcatctcgttggtgggctaacttaggtttcgctgataacttaaaattcgctcgtgatcgtttagttgaatgtttttcttgcgcagtaggcgtagcatttgaacctgaacactcttcttttcgtatctgtttaacaaaagttattaatttagttttaataattgatgacgtatacgacatatatggaagtgaagaagaattaaaacactttacaaatgctgttgatcgttgggattctcgtgaaacagaacaattaccagaatgtatgaaaatgtgctttcaagttttatacaatactacatgtgaaattgctcgtgaaattgaagaagaaaatggatggaatcaagttttacctcaattaactaaagtatgggctgatttttgtaaagcattattagtagaagctgaatggtacaataaaagtcacatcccaactttagaagaatatcttcgtaatggctgtatttcatcaagtgtttctgtattattagtacattctttctttagtattacacatgaaggtacaaaagaaatggcagatttcttacacaaaaacgaagacttattatacaacatctcattaattgtacgtttaaacaacgacttaggtacaagtgcagctgaacaagaacgtggtgattcaccatcatctattgtatgttacatgcgtgaagttaatgctagtgaagaaacagctcgtaaaaatataaaaggaatgatcgacaatgcttggaaaaaagttaatggtaaatgttttacaactaatcaagttccttttctttcttcttttatgaataacgctactaatatggctcgtgtagctcattcattatataaagacggagacggttttggcgatcaggaaaaaggtccacgtactcacatcttatctttattattccaaccattagttaacggtaccggtgaaaacttatactttcaaggttctggtggtggtggttctgactacaaagatgacgatgacaaaggaaccggttaa

SEQ ID NO: 112 - DNA, 1773 bp, Artificial Sequence
  Description of Artificial Sequence: Synthetic polynucleotide
atggtaccaagtagtaatgtatcagctattcctaattcttttgaattaattcgtcgttcagctcaatttcaggcttctgtatggggtgattactttttatcttatcactctttaccacctgagaaaggtaataaagtaatggaaaaacaaactgaagaacttaaagaggaaatcaaaatggaattagtttctactactaaagatgaaccagagaaattacgtttaattgaccttattcaacgtttaggtgtatgttatcactttgaaaatgaaattaacaacattttacaacaattacaccacattactattacttctgagaaaaacggtgacgataatccttataacatgactttatgtttccgtttattacgtcaacaaggttacaatgtatctagtgaaccttttgatcgttttcgtggcaaatgggaatcttcttatgataacaatgtagaagaacttttatcattatatgaagcatctcaattaagaatgcaaggtgaagaagcattagatgaagcattctgttttgcaactgcacaattagaagctattgttcaagatcctactacagatccaatggttgcagcagaaatcagacaagcattaaaatggccaatgtacaaaaacttacctcgtttaaaagctcgtcatcatattggtttatattctgagaaaccatggcgtaatgagtcattacttaatttcgcaaaaatggacttcaataaacttcaaaatttacatcaaactgaaattgcatatatttctaaatggtgggacgattacggctttgcagaaaaactttctttcgcacgtaatcgtattgttgaaggctatttcttcgcattaggtatctttttcgaacctcaacttttaacagcacgtcttataatgacaaaagtaatcgctattggttctatgttagatgacatttatgatgtttatggtacttttgaagagttaaaacttttaacattagctttagaacgttgggataaatcagaaacaaaacaattacctaattacatgaaaatgtactacgaagcattattagatgtttttgaagaaattgagcaagaaatgtcacaaaaagaaactgaaacaacaccatactgtattcatcacatgaaagaagctactaaagaacttggacgtgtatttttagttgaagcaacttggtgtaaagaaggttatactcctaaagtagaggaatacttagacattgctttaatttcttttggtcataaattacttatggtaactgctttattaggtatgggttctcacatggctacacaacaaattgtacaatggattacatctatgccaaatatcttaaaagcatctgcagtaatatgtcgtttaatgaatgacattgtatctcataaatttgaacaagaacgtggtcatgttgcttctgctatcgaatgctacatggaacaaaaccaccttagtgaatatgaagcattaattgctcttcgtaaacaaattgatgatttatggaaagacatggtagaaaattactgtgcagtaatcacagaagacgaagtacctcgtggtgttttaatgcgtgttttaaatcttacacgtttattcaatgttatttacaaagacggtgatggatacacacaaagtcatggtagtacaaaagctcacattaaaagtcttttagttgatagtgtacctcttggtaccggtgaaaatctttactttcaaggttcaggtggaggtggttctgattataaagatgatgatgacaaaggaaccggttaa

SEQ ID NO: 113 - DNA, 1770 bp, Artificial Sequence
  Description of Artificial Sequence: Synthetic polynucleotide
atggtaccaaaagacatgagtattccattattagcagctgtatcttctagtacagaagaaacagtacgtcctatcgcagattttcatccaacactttggggtaatcattttcttaaatctgctgctgacgtagaaactattgatgcagcaacacaagagcaacacgctgcattaaaacaagaagtacgtcgtatgattactacaacagcaaataaacttgcacaaaaacttcacatgattgatgctgtacaacgtttaggtgttgcttatcattttgaaaaagaaattgaagacgaattaggtaaagtaagtcacgatttagattcagatgatttatacgttgtatctttacgttttcgtttattccgtcaacaaggtgtaaaaattagttgcgatgttttcgacaaattcaaagatgacgaaggaaaattcaaagagtctcttattaacgatattagaggaatgttatcattatacgaagcagcttacttagctattagaggtgaagatattttagacgaagcaattgttttcacaactactcacttaaaaagtgttatctctattagtgatcattcacatgctaatagtaatttagctgaacaaatacgtcatagtttacaaattccacttcgtaaagctgctgcaagattagaagcacgttatttcttagatatttactctcgtgatgatttacatgatgaaacattacttaaattcgctaaacttgactttaacattcttcaagctgcacaccaaaaagaagctagtattatgactcgttggtggaacgatttaggttttcctaaaaaagttccttatgctcgtgaccgtattatagaaacttatatttggatgttattaggagtttcatacgaacctaatttagcatttggaagaatttttgcaagtaaagtagtatgtatgattacaacaattgatgatacatttgatgcttatggtacatttgaagagttaacattattcactgaagctgttacacgttgggatattggtttaattgacacattacctgaatatatgaaattcattgtaaaagctcttttagacatttaccgtgaagctgaagaagaattagctaaagaaggtagatcatacggtattccatacgctaaacaaatgatgcaagagttaatcattttatactttactgaggctaaatggttatacaaaggttacgttcctacatttgacgaatacaaaagtgtagctttacgttctattggtcttagaacattagcagtagcttcatttgtagatttaggtgactttattgctacaaaagacaattttgaatgtattcttaaaaatgcaaaaagtttaaaagctactgaaacaattggccgtttaatggatgatatagctggttacaaatttgaacagaaacgtggtcataacccatctgctgttgagtgttacaaaaatcaacacggagtatcagaagaagaagcagttaaagagcttttattagaagttgcaaacagttggaaagatattaacgaggaacttttaaatccaactacagttccattacctatgttacagcgtttattatattttgctcgttcaggtcacttcatctatgatgatggacatgatcgttatacacattctttaatgatgaaaagacaagttgcacttttattaactgaacctttagctattggtaccggtgaaaacttatactttcaaggttcaggtggtggtggatctgattataaagatgatgatgacaaaggaaccggttaa

SEQ ID NO: 114 - DNA, 1830 bp, Artificial Sequence
  Description of Artificial Sequence: Synthetic polynucleotide
atggtaccagatttagctgttgagattgcaatggacttagctgttgatgacgttgagcgtcgtgtaggtgactatcatagtaacctttgggatgatgattttattcagagtttatcaacaccatacggcgcatcatcatatcgtgaacgtgctgaaagattagtaggagaagttaaagaaatgtttacttctatttctatcgaagatggtgaacttacatctgatttattacaacgtttatggatggtagataatgtagagcgtttaggcatttcacgtcatttcgagaacgaaataaaagcagctattgattatgtttattcatattggagtgacaaaggtattgtacgtggtcgtgattcagctgttcctgacttaaatagtattgctttaggttttcgtacattacgtttacacggttacacagttagtagtgatgtatttaaagttttccaagatcgtaaaggtgaatttgcttgcagtgcaattccaactgaaggagatattaaaggagttttaaacttacttcgtgcaagttatattgcattccctggtgaaaaagtaatggaaaaagctcaaacttttgcagcaacataccttaaagaagcattacagaaaattcaagtaagtagtttaagtcgtgaaatcgaatatgttcttgaatacggttggttaactaactttcctcgtttagaagcacgtaactatattgacgtattcggtgaagaaatttgtccatacttcaaaaaaccatgtattatggttgacaaacttttagaattagcaaaattagaatttaacttatttcacagtcttcaacaaacagagttaaaacatgttagtcgttggtggaaagatagtggtttctctcaattaacatttacaagacaccgtcatgttgagttttatacattagctagttgtatagcaattgaaccaaaacacagtgcttttcgtcttggttttgctaaagtttgttatttaggtatagttttagatgatatttatgacacatttggtaaaatgaaagaattagaactttttactgcagcaatcaaacgttgggacccttctactacagaatgcttacctgaatacatgaaaggtgtttatatggctttttacaattgtgttaatgaattagcacttcaagcagagaaaacacaaggtcgtgatatgttaaactatgcacgtaaagcatgggaagctctttttgatgcatttttagaagaagcaaaatggatctcttctggctatttaccaacattcgaagaatacttagaaaatggtaaagtatcttttggttatcgtgctgctacattacaaccaattttaacattagatattcctttacctttacatattttacaacagattgattttccaagtcgttttaatgatttagcttcatctattttacgtttaagaggtgatatctgtggttaccaagctgaacgtagtcgtggtgaagaagcatcatcaatttcatgttatatgaaagataatccaggttctactgaagaagatgcattatctcacattaatgcaatgatctcagacaatattaacgaattaaactgggaacttttaaaaccaaattcaaatgtaccaatttcatcaaaaaaacatgcatttgacattcttcgtgctttctatcacttatacaaatatcgtgatggcttctctatcgcaaaaattgaaactaaaaatcttgtaatgcgtacagttttagaacctgtaccaatgggtaccggtgaaaacttatactttcagggttctggtggaggtggttcagactataaagatgatgatgataaaggaaccggttaa

SEQ ID NO: 115 - DNA, 2511 bp, Artificial Sequence
  Description of Artificial Sequence: Synthetic polynucleotide
atggtaccaacaagtgtatcagtagaatcaggaacagtatcttgtttatcatcaaacaacttaattagacgtacagctaatccacatcctaacatttggggatatgattttgttcactcacttaaatcaccatatacacacgactcatcatatcgtgaacgtgctgagactttaatttcagaaataaaagttatgcttggaggtggtgaattaatgatgactccatcagcttatgatacagcatgggtagctcgtgttccatcaattgacggtagtgcttgtccacaatttccacaaactgttgaatggattcttaaaaaccaattaaaagatggtagttggggaactgaatctcacttcttacttagtgacagattattagctacattaagttgtgtattagcattattaaaatggaaagtagctgatgttcaagtagagcaaggtattgagtttatcaaacgtaatttacaagctattaaagacgaacgtgatcaagacagtttagtaactgatttcgagattattttcccatcacttttaaaagaggctcaatctttaaacttaggcttaccttatgatttaccatatattagattattacaaacaaaacgtcaagaacgtcttgctaacttaagtatggataaaattcacggtggtactttattatcatctttagagggcattcaagatatagttgaatgggaaacaattatggatgtacaatctcaagatggttctttcttatcatcaccagcttctacagcatgtgtattcatgcatacaggagatatgaaatgtttagatttcttaaacaacgtattaactaaatttggtagtagtgttccttgtttataccctgtagatttattagaacgtcttttaattgtagataatgtagagcgtcttggtattgaccgtcattttgaaaaagaaatcaaagaggctttagattatgtttatcgtcattggaacgatcgtggtattggttggggtcgtttatcacctatcgcagacttagaaacaacagctttaggttttcgtttacttcgtcttcatcgttacaatgtttctcctgtagtattagacaatttcaaagacgcagatggcgagttcttctgcagtacaggtcaatttaacaaagatgttgcaagtatgttatctttataccgtgcttctcaattagctttccctgaagaatcaattttagatgaagctaaatcattctcaacacaatatcttcgtgaagcattagaaaaatcagaaacattttcttcttggaatcatcgtcagagtttatcagaagaaattaaatatgctttaaaaacatcatggcacgcttcagttcctcgtgttgaagcaaaacgttattgtcaggtttaccgtcaagactatgctcatttagcaaaatcagtttataaacttcctaaagtaaataatgagaaaattcttgaattagcaaaattagattttaacattattcaatctatccatcaaaaagaaatgaaaaatgttacatcatggtttcgtgattcaggcttaccacttttcacatttgctcgtgaaagacctttagagttttactttttaatcgctggtggaacatacgaacctcaatacgcaaaatgtagattcttatttacaaaagtagcttgtttacaaactgttttagacgatatgtacgatacttacggtacaccatcagagttaaaattatttactgaggcagttcgtcgttgggatttatcattcacagaaaacttacctgattatatgaaattatgctacaaaatttactatgatattgttcatgaagttgcttgggaagtagaaaaagaacagggacgtgagcttgtttcatttttccgtaaaggttgggaagactatcttttaggttattatgaagaagctgaatggttagctgctgaatacgttcctactttagatgaatacattaaaaacggtattacatctattggtcaacgtattttacttttatcaggtgtacttattatggaaggtcaacttttatcacaagaagctcttgaaaaagtagattatccaggtcgtcgtgttttaacagaattaaacagtttaattagtcgtttagcagacgatactaaaacatacaaagcagaaaaagctcgtggtgaacttgctagtagtattgaatgttatatgaaagaccaccctggttgtcaagaagaagaagcattaaaccatatttatggcattttagaaccagctgttaaagaattaactcgtgagtttcttaaagcagatcacgtaccattcccttgcaaaaaaatgttatttgatgaaacaagagttacaatggtaattttcaaagatggtgatggtttcggtatttctaaattagaagtaaaagaccacataaaagaatgtttaattgagccattaccacttggtaccggtgaaaatctttattttcaaggtagtggtggtggcggttctgactacaaagatgacgacgataaaggaaccggttaa

SEQ ID NO: 116 - DNA, 1731 bp, Artificial Sequence
  Description of Artificial Sequence: Synthetic polynucleotide
atggtaccaggttctgaagtaaatagacctttagcagactttccagcaaacatttgggaagacccattaacttctttctcaaaatctgatcttggtacagaaacatttaaagagaaacatagtactttaaaagaagctgttaaagaggcatttatgagttctaaagctaatccaatcgaaaatatcaaattcatagatgcattatgccgtttaggagtatcttatcactttgaaaaagatattgtagaacaattagataaatcatttgattgcttagattttccacaaatggtacgtcaagaaggttgcgatttatatacagttggtattatctttcaagtttttagacaatttggtttcaaattaagtgctgatgtttttgaaaaattcaaagatgaaaatggtaaattcaaaggtcacttagtaactgatgcttatggtatgttatcattatacgaagctgcacaatggggtactcacggtgaagacatcattgacgaagctcttgctttttctcgtagtcacttagaagaaatatctagtcgtagttcaccacacttagcaattcgtattaaaaacgctttaaaacatccatatcataaaggtatttcacgtattgaaacacgtcaatacattagttactatgaagaagaagaatcttgtgatccaacattattagagttcgctaaaattgactttaacttattacaaattttacaccgtgaagagttagcttgtgtaactcgttggcatcatgaaatggaatttaaaagtaaagtaacttacacacgtcatcgtattacagaagcatatttatggagtcttggaacatattttgaaccacaatacagtcaagctcgtgtaataactacaatggcattaatcttatttactgctttagacgacatgtacgatgcttacggtactatggaggagttagagttattcacagatgctatggacgaatggttaccagttgttccagatgaaattcctattccagattcaatgaaattcatttacaatgttacagttgaattttacgataaattagacgaagaattagaaaaagaaggtcgttctggttgtggtttccatcttaaaaaaagtttacaaaaaacagctaatggatatatgcaagaagcaaaatggcttaaaaaagattacattgctacatttgatgagtataaagaaaatgctattttatcttcaggttattatgcattaattgcaatgacatttgttcgtatgactgatgttgctaaattagatgcttttgaatggttaagtagtcacccaaaaattcgtgtagcaagtgaaatcatttcacgttttacagacgatatttcaagttatgaatttgaacacaaacgtgaacacgttgctacaggtattgattgttatatgcaacaattcggagttagtaaagaacgtgctgttgaagttatgggcaatatagtttctgatgcatggaaagacttaaatcaagaacttatgcgtcctcatgttttcccatttccacttcttatgcgtgttttaaatctttcaagagtaattgatgtattttatcgttaccaagatgcatatactaatccaaaattacttaaagagcacattgtttctttacttattgaaactattccaattggtaccggtgaaaacttatactttcaaggtagtggtggaggtggttctgattataaagacgacgatgacaaaggaaccggttaa

SEQ ID NO: 117 - DNA, 1773 bp, Artificial Sequence
  Description of Artificial Sequence: Synthetic polynucleotide
atggtaccagaggcaattagagtatttggcttaaaacttggttcaaaattatctattcactcacaaacaaatgcttttcctgcattcaaattatctcgttttccattaacatctttccctggtaaacatgctcacttagatccattaaaagcaacaactcatccattagcttttgatggtgaagaaaataaccgtgagtttaaaaacttaggtccaagtgagtggggccatcaatttctttctgctcatgtagatttatctgaaatggatgcattagaacgtgaaattgaagctcttaaaccaaaagtacgtgatatgttaatatcaagtgaaagttcaaaaaaaaaaatcttatttctttatcttttagtatcattaggattagcttatcactttgaagatgaaattaaagaaagtttagaggatggattacagaaaattgaggaaatgatggcttcagaagatgatcttcgttttaaaggcgataatggtaaattcaaagaatgtttagcaaaagatgctaaaggtattttatctctttatgaggctgctcacatgggtacaacaactgattatattcttgatgaggctttatcatttactttaacatatatggaatcattagcagcttcaggaacatgtaaaatcaacttatcacgtcgtattagaaaagcattagatcaacctcaacacaaaaatatggaaataattgtagcaatgaaatacattcaattttatgaagaagaggaagattgcgataaaactttacttaaatttgctaaacttaactttaaattcttacaattacactatttacaagaacttaaaatcttatctaaatggtataaagaccaagactttaaatcaaaattacctccatatttccgtgaccgtcttgtagaatgtcattttgcatcattaacatgttttgagcctaaatatgctcgtgcacgtattttcttatctaaaatcttcactgttcaaattttcattgacgatacttgtgaccgttacgcatcattaggtgaagttgagtcattagctgacactatcgaacgttgggaccctgatgatcatgctatggacggattacctgattatcttaaatcagtagttaaatttgtattcaatacatttcaagaatttgaacgtaaatgtaaacgttcacttcgtattaacttacaagtagcaaaatgggttaaagctggtcacttaccatcttttgatgagtatcttgatgtagctggtttagaattagctatttcattcactttcgctggtatcttaatgggcatggaaaatgtttgtaaacctgaagcatacgaatggttaaaatctcgtgacaaacttgttcgtggtgtaatcacaaaagttcgtttacttaatgatatttttggctatgaagatgatatgcgtcgtggttatgtaacaaattcaataaactgctacaaaaaacaatatggagtaacagaggaagaagctattcgtaaattacatcaaatcgttgctgatggagagaaaatgatgaatgaagagttcttaaaacctattaatgtaccatatcaggttcctaaagtagttattttagacactttacgtgcagctaatgtttcatacgaaaaagatgacgaatttacacgtccaggcgaacaccttaaaaactgcattacatctatttacttcgatttaggtaccggtgaaaacttatactttcaaggtagtggtggcggtggtagtgattacaaagatgatgatgataaaggaaccggttaa

SEQ ID NO: 118 - DNA, 1128 bp, Artificial Sequence
  Description of Artificial Sequence: Synthetic polynucleotide
atggtaccaactacaacattatcatctaaccttaactcacaattcatgcaggtttacgagactcttaaatcagaacttattcatgacccattatttgagttcgatgacgattcaagacaatgggtagaacgtatgattgattatactgtaccaggtggtaaaatggttcgtggttatagtgtagtagatagttatcaattacttaaaggtgaagaacttacagaagaagaggcatttttagcttgtgcacttggttggtgtacagaatggtttcaagcattcattcttttacatgatgatatgatggatggtagtcacacaagacgtggtcaaccatgttggtttcgtttacctgaggttggtgctgttgctattaatgatggtgttttacttcgtaatcacgttcaccgtattcttaaaaaacattttcaaggtaaagcatattatgttcatttagttgatttattcaatgaaactgaatttcaaacaattagtggacaaatgatcgacttaattacaacattagttggtgaaaaagacttatctaaatattcattaagtattcatcgtcgtatcgttcaatacaaaacagcatactactcattttacttaccagttgcttgtgctttacttatgtttggtgaggatcttgataaacatgtagaagttaaaaatgttcttgttgaaatgggtacatattttcaagttcaagatgattatttagattgttttggtgctccagaagttattggcaaaattggtactgatattgaagactttaaatgttcatggttagtagttaaagcattagaattagcaaatgaagaacagaaaaaaactttacacgaaaattatggaaaaaaagatccagcatcagttgctaaagttaaagaagtataccacacacttaatttacaagctgttttcgaagattatgaagcaacatcatacaaaaaacttattacttctattgaaaatcacccatctaaagctgttcaagctgttttaaaatctttcttaggcaaaatatacaaacgtcaaaaaggtaccggtgaaaacttatactttcaaggttctggtggcggtggaagtgattacaaagatgatgacgataaaggaaccggttaa

SEQ ID NO: 119 - DNA, 1803 bp, Artificial Sequence
  Description of Artificial Sequence: Synthetic polynucleotide
atggtaccaagtcaaccttactgggctgcaattgaagcagacattgaaagatatttaaaaaaatcaattacaattcgtccaccagaaactgtatttggtcctatgcaccatttaacatttgctgctcctgctactgcagctagtacattatgccttgctgcttgtgaattagttggcggtgatcgtagtcaagctatggcagctgctgctgctatccatttagttcatgcagctgcttacgttcacgaacatcttcctttaacagatggatcacgtcctgtaagtaaacctgctattcaacataaatatggtccaaacgttgaacttttaacaggtgatggtatcgttcctttcggttttgagttattagcaggttcagtagatccagcacgtactgatgaccctgatcgtattttacgtgtaattattgaaatttctcgtgctggtggaccagaaggcatgatttctggtttacaccgtgaggaagaaatcgtagatggtaacacatcattagactttatagaatatgtatgcaaaaaaaaatacggtgaaatgcacgcatgtggtgcagcttgcggagctattttaggtggagctgctgaagaagaaattcaaaaacttcgtaactttggtctttatcaaggcacattacgtggtatgatggaaatgaaaaatagtcatcagttaattgacgaaaatatcattggaaaacttaaagaacttgctcttgaagaattaggtggattccacggtaaaaacgctgaattaatgagttctttagttgctgaacctagtttatatgcagcttcatcaaataacttaggtatcgaaggtcgttttgactttgacggttacatgcttcgtaaagcaaaatctgtaaataaagcattagaagctgctgttcaaatgaaagaaccacttaaaattcacgaatcaatgcgttattcattattagctggtggtaaacgtgttcgtccaatgttatgtattgcagcttgtgaacttgttggtggtgacgaatctacagcaatgcctgcagcatgtgctgttgaaatgattcacacaatgtctttaatgcatgatgaccttccatgtatggataacgatgacttacgtcgtggtaaacctacaaaccacatggcttttggtgagtctgtagctgttcttgctggtgatgcattacttagttttgcttttgaacatgttgctgctgcaacaaaaggcgcaccacctgaacgtatcgtacgtgtattaggtgaattagctgttagtattggttcagaaggacttgtagcaggtcaagttgtagacgtttgttctgaaggcatggctgaagtaggattagatcatcttgaatttattcaccatcataaaactgctgcattattacaaggttcagttgttttaggtgcaatattaggaggcggtaaagaagaagaagtagctaaacttcgtaaatttgctaactgtattggtttacttttccaagttgttgatgatattttagatgttactaaaagtagtaaagagttaggtaaaactgcaggtaaagacttagtagctgataaaactacatatcctaaacttataggcgttgaaaaatcaaaagaatttgctgaccgtttaaatcgtgaagcacaagaacaattattacattttcatcctcaccgtgctgctccattaatcgctttagctaactacatcgcttaccgtgataatggtaccggtgaaaacttatacttccagggtagtggtggtggcggatcagattataaagatgacgatgataaaggaaccggttaa

SEQ ID NO: 120 - DNA, 1371 bp, Artificial Sequence
  Description of Artificial Sequence: Synthetic polynucleotide
atggtaccagtaacagcagcacgtgcaacaccaaaattaagtaatagaaaattacgtgttgctgtaattggaggcggtccagcaggaggtgcagctgctgaaacattagcacaaggaggtattgaaacaattcttatcgaacgtaaaatggataattgtaaaccatgtggtggtgctattccattatgtatggtaggagagttcaatttacctttagacattattgaccgtcgtgtaacaaaaatgaaaatgatctctccttcaaacattgcagttgatatcggtcgtacacttaaagaacacgaatatattggtatggttcgtcgtgaggtacttgatgcttatcttcgtgaacgtgcagaaaaatcaggtgctactgttattaacggtttattcttaaaaatggatcacccagaaaattgggattcaccatatacacttcactacacagagtatgatggaaaaacaggtgctacaggaactaaaaaaactatggaagtagatgctgttattggtgctgatggtgctaattctcgtgttgcaaaaagtattgacgcaggtgattatgattatgctattgcatttcaagaacgtattcgtatacctgatgagaaaatgacttattatgaggacttagctgagatgtatgtaggtgatgatgtatcaccagacttctacggttgggtattcccaaaatgtgatcatgtagctgttggtacaggtactgtaacacataaaggtgatatcaaaaaattccagttagctacacgtaatcgtgctaaagataaaattcttggtggcaaaataatccgtgtagaggctcatcctattccagagcatcctagaccacgtcgtttatcaaaacgtgttgcattagtaggcgacgcagcaggttacgttactaaatgttcaggagaaggaatttacttcgcagctaaatctggtcgtatgtgtgctgaagctatcgttgaaggttcacaaaatggcaaaaaaatgatagatgaaggcgatttaagaaaatacttagaaaaatgggataaaacttacttaccaacttatcgtgttttagatgtacttcaaaaagttttctatcgttctaacccagctcgtgaggcttttgttgaaatgtgtaacgatgagtatgtacagaaaatgacatttgattcttacctttataaacgtgtagctcctggtagtccattagaagatatcaaattagctgtaaatactattggttcacttgttcgtgctaacgcattacgtcgtgaaattgagaaattatcagtaggtaccggtgagaatctttactttcaaggatcaggtggtggtggttctgattataaagatgacgatgataaaggaaccggttaa

SEQ ID NO: 121 - DNA, 1362 bp, Artificial Sequence
  Description of Artificial Sequence: Synthetic polynucleotide
atggtaccagtagctgttattggtggtggtccaagtggcgcttgtgcagcagaaactttagcaaaaggtggtgtagaaactttcttacttgagcgtaaattagataattgtaaaccttgtggaggtgcaattccattatgtatggttgaagaatttgatttaccaatggaaataattgaccgtcgtgttactaaaatgaaaatgatatcaccttcaaaccgtgaagttgatgttggaaaaactttatcagaaactgaatggatcggtatgtgtcgtcgtgaagtatttgacgattacttaagaaaccgtgcacagaaattaggtgctaatattgttaacggtttattcatgcgttcagaacaacaatctgcagagggtccattcacaattcactataattcttatgaagacggtagtaaaatgggaaaacctgctactttagaagttgatatgataattggtgcagatggagcaaattctcgtattgcaaaagagatagatgcaggtgaatacgactacgctatagcttttcaagaacgtattcgtattcctgatgataaaatgaaatattacgaaaaccttgctgaaatgtatgtaggtgatgacgtatctcctgatttctatggttgggtttttcctaaatatgatcacgttgctgttggtacaggtactgttgtaaacaaaacagctattaaacaatatcaacaggcaacacgtgacagatcaaaagttaaaacagaaggtggcaaaattatacgtgttgaagcacacccaattccagaacatccacgtccacgtcgttgtaaaggtcgtgttgcattagtaggcgacgcagctggttatgttacaaaatgttctggcgagggcatttactttgctgctaaatctggtagaatggctgctgaagctattgtagaaggttctgctaacggtacaaaaatgtgtggtgaggatgcaattcgtgtttatttagataaatgggatcgtaaatattggacaacatacaaagtattagacattttacaaaaagtattttatcgtagtaatccagcacgtgaagcatttgttgaattatgtgaagatagttatgtacagaaaatgacatttgattcatacttatataaaactgttgttccaggaaacccattagacgacgtaaaattacttgttcgtacagtatcttctattttacgttcaaatgctttacgttctgttaattctaaatctgtaaatgtttctttcggctctaaagcaaatgaggaacgtgttatggctgcaggtaccggtgaaaatctttattttcaaggttcaggaggtggtggttcagattataaagatgatgatgacaaaggaaccggttaa

SEQ ID NO: 122 - DNA, 918 bp, Artificial Sequence
  Description of Artificial Sequence: Synthetic polynucleotide
atggtaccagcaatggcagtaccattagatgtagtaattacatatccttcttcaggtgctgctgcttatccagtacttgttatgtataacggtttccaagctaaagctccatggtatcgtggtattgtagatcatgtttctagttggggttacacagttgttcaatatacaaatggtggcttatttcctattgttgtagatcgtgttgagttaacttatttagagccattattaacttggttagaaacacaaagtgctgatgctaaatctcctttatacggtcgtgcagatgtttctcgtttaggtacaatgggtcattcacgtggtggtaaattagcagctttacaatttgctggacgtacagatgtaagtggttgtgtattatttgaccctgtagatggaagtccaatgacaccagaatctgctgattatccttcagctacaaaagcattagcagcagctggtcgttctgctggcttagtaggtgcagctattacaggttcatgtaatccagtaggtcaaaattacccaaaattctggggtgctttagctcctggttcttggcaaatggtattatcacaagctggtcacatgcaatttgctcgtactggtaatccattcttagattggtcattagaccgtttatgtggtcgtggtacaatgatgagttcagatgttattacatatagtgcagcatttactgttgcttggtttgaaggtatttttcgtcctgctcaaagtcaaatgggtatttctaatttcaaaacttgggctaatactcaagttgcagctcgtagtatcacttttgatattaaacctatgcaatctcctcagggtaccggtgaaaacctttactttcaaggtagtggtggtggaggaagtgattataaagatgatgatgacaaaggaaccggttaa

SEQ ID NO: 123 - DNA, 948 bp, Artificial Sequence
  Description of Artificial Sequence: Synthetic polynucleotide
atggtaccagcaccaccaaaaccagttcgtataacttgtccaacagtagctggcacttatcctgttgttttattctttcacggtttttatcttcgtaactatttctattcagatgttttaaatcatattgctagtcatggttacatcttagttgcaccacaattatgtaaacttttacctccaggtggccaagtagaagttgatgacgctggttcagttattaactgggcttcagagaatcttaaagcacaccttccaacttctgttaatgctaatggtaaatatacatctttagttggacattcacgtggtggcaaaacagctttcgcagttgcattaggtcacgcagctacattagatccatcaattacattttcagcattaattggtattgatccagtagcaggaactaacaaatacattcgtacagatccacacatcttaacttataaacctgaatcatttgaattagatattcctgtagctgttgtaggcactggtcttggtccaaaatggaataacgtaatgcctccatgcgcacctacagatttaaaccacgaagaattttacaaagaatgtaaagctactaaagctcactttgttgctgctgattatggtcacatggacatgttagacgacgatcttccaggttttgtaggcttcatggctggttgtatgtgtaaaaatggtcaacgtaaaaaatcagaaatgcgttcttttgtaggtggtatagttgtagcattcttaaaatattctttatggggtgaaaaagctgaaataagattaattgttaaagatcctagtgtatctcctgctaaattagacccatcaccagaattagaagaagcatcaggtatttttgttggtaccggtgaaaatctttattttcaaggttcaggtggaggtggttctgattataaagatgatgatgacaaaggaaccggttaa

SEQ ID NO: 124 - DNA, 906 bp, Artificial Sequence
  Description of Artificial Sequence: Synthetic polynucleotide
atggtaccagctacaccagttgaagaaggtgattatccagttgtaatgttattacatggctaccttttatataattcattttattcacaattaatgttacatgtatcatctcacggtttcatcttaattgctccacaattatactcaattgctggtcctgatactatggatgaaattaaaagtactgctgagattatggactggttatcagttggtttaaatcactttttaccagctcaagttacacctaatttatctaaatttgcattatctggtcatagtcgtggtggtaaaactgcttttgctgtagcattaaaaaaatttggttattcttcaaacttaaaaattagtactttaattggtattgatccagtagacggaacaggtaaaggtaaacaaactccacctcctgttttagcatatttacctaatagttttgacttagacaaaacaccaattttagtaattggttcaggtttaggtgaaactgcacgtaatcctttatttcctccatgtgctcctccaggtgttaaccaccgtgagtttttccgtgaatgtcaaggtccagcatggcactttgttgctaaagattatggtcatttagacatgcttgatgatgatacaaaaggtattcgtggcaaatctagttactgtttatgcaaaaatggtgaagaacgtcgtccaatgcgtcgtttcgttggtggtttagttgttagttttcttaaagcatatcttgaaggtgatgatcgtgaattagtaaaaatcaaagatggttgtcatgaagatgtacctgttgaaattcaagaatttgaagtaattatgggtaccggtgaaaatctttactttcaaggttcaggcggtggaggttcagattataaagatgatgatgacaaaggaaccggttaa

SEQ ID NO: 125 - DNA, 1605 bp, Artificial Sequence
  Description of Artificial Sequence: Synthetic polynucleotide
atggtaccaagtcacaaaaaaaaaaacgtaatcttcttcgtaactgatggtatgggtcctgcttctctttcaatggctcgttcatttaatcaacacgttaatgatttaccaattgatgatattttaacattagatgaacattttattggaagttcaagaacacgttcatcagattcacttgtaactgactcagctgctggagctacagcttttgcttgtgcacttaaatcatacaatggtgctataggtgtagatccacaccatcgtccatgtggaactgttttagaagctgctaaattagcaggttatttaacaggattagtagttactacacgtattactgatgctacaccagctagtttctcaagtcacgtagattatcgttggcaagaagatttaattgcaacacaccaattaggtgaatatcctttaggacgtgttgttgatcttcttatgggtggtggtcgttctcacttttatcctcaaggtgaaaaagctagtccatacggtcaccacggtgcacgtaaagatggtcgtgatttaatcgatgaagctcaaagtaatggctggcagtatgtaggagatcgtaaaaattttgattctttacttaaatcacatggtgaaaatgttactttaccatttttaggtttatttgctgacaacgatatcccatttgaaattgatcgtgatgaaaaagaatatcctagtttaaaagaacaagtaaaagtagcattaggtgctttagaaaaagcaagtaacgaagataaagatagtaatggtttctttttaatggtagaaggttctcgtattgatcatgctggccatcaaaacgatcctgcatctcaagtacgtgaagtattagcatttgatgaggcttttcaatatgtattagaatttgcagaaaacagtgatacagaaacagtattagtaagtacatcagatcatgaaacaggtggtttagttacttcaagacaagtaacagcatcatacccacaatatgtatggtatcctcaagtattagctaacgctacacatagtggagagtttcttaaacgtaaattagttgatttcgttcatgaacacaaaggcgcatcatcaaaaatagaaaacttcataaaacacgaaattcttgaaaaagatttaggtatttatgattatacagattctgacttagaaacacttattcatttagatgataacgctaatgcaattcaagataaacttaatgatatggtaagttttagagctcaaattggttggacaacacatggtcattcagcagttgatgtaaacatatatgcttacgcaaacaaaaaagctacatggtcttatgttcttaataacttacaaggtaatcacgaaaacacagaagttggtcaattcttagagaatttcttagaattaaacttaaatgaagttactgatttaatccgtgatacaaaacatacttctgattttgacgcaacagaaatagcaagtgaggttcaacactatgatgaatattaccacgaattaacaaatggtaccggtgaaaatctttattttcaaggttctggtggaggtggcagtgattataaagatgatgatgacaaaggaaccggttaa

SEQ ID NO: 126 - DNA, 1191 bp, Artificial Sequence
  Description of Artificial Sequence: Synthetic polynucleotide
atggtaccacacaagttcacaggtgttaacgctaaattccagcaaccagcattaagaaatttatctccagtggtagttgagcgcgaacgtgaggaatttgtaggattctttccacaaattgttcgtgacttaactgaagatggtattggtcatccagaagtaggtgacgctgtagctcgtcttaaagaagtattacaatacaacgcacctggtggtaaatgcaatagaggtttaacagttgttgcagcttaccgtgaactttctggaccaggtcaaaaagacgctgaaagtcttcgttgtgctttagcagtaggatggtgtattgaattattccaagcctttttcttagtttgggacgatataatggaccagtcattaactagacgtggtcaattatgttggtacaagaaagaaggtgttggtttagatgcaataaatgattcttttcttttagaaagctctgtgtatcgcgttcttaaaaagtattgccgtcaacgtccatattatgtacatttattagagctttttcttcaaacagcttaccaaacagaattaggacaaatgttagatttaatcactgctcctgtatctaaggtagatttaagccatttctcagaagaacgttacaaagctattgttaagtataaaactgctttctattcattctatttaccagttgcagcagctatgtatatggttggtatagattctaaagaagaacatgaaaacgcaaaagctattttacttgagatgggtgaatacttccaaattcaagatgattatttagattgttttggcgatcctgctttaacaggtaaagtaggtactgatattcaagataacaaatgttcatggttagttgtgcaatgcttacaaagagtaacaccagaacaacgtcaacttttagaagataattacggtcgtaaagaaccagaaaaagttgctaaagttaaagaattatatgaggctgtaggtatgagagccgcctttcaacaatacgaagaaagtagttaccgtcgtcttcaagagttaattgagaaacattctaatcgtttaccaaaagaaattttcttaggtttagctcagaaaatatacaaacgtcaaaaaggtaccggtgaaaacttatactttcaaggctcaggtggcggtggaagtgattacaaagatgatgatgataaaggaaccggttaa

SEQ ID NO: 127 - DNA, 1734 bp, Artificial Sequence
  Description of Artificial Sequence: Synthetic polynucleotide
catatggtaccaagacgttcaggtaactataatcctagccgttgggacgtaaatttcattcaatctttattatctgattataaagaagataaacacgttattagagcttctgaattagtaacacttgttaagatggaattagaaaaagaaacagatcaaatccgtcaattagaattaattgacgatttacaacgtatgggtttatctgatcatttccaaaacgaatttaaagaaatcttatcaagtatttacttagatcatcattattacaaaaatccatttccaaaagaagagcgtgatttatactcaactagcttagcttttcgtttattacgtgaacacggttttcaagtagcacaagaagtttttgattcattcaaaaatgaagagggtgaatttaaggagagcttatctgacgatactcgtggcttattacaattatatgaagcatcattcttattaacagagggtgaaacaaccttagaaagtgcacgcgaatttgctacaaaatttttagaagaaaaagttaacgaaggtggcgttgatggtgacttattaacaagaattgcttactcattagatattcccttacattggcgcattaaacgtcctaatgccccagtttggattgaatggtatcgtaaacgtccagatatgaacccagtggttttagaattagcaattttagacttaaacattgtacaagctcaatttcaagaggaattaaaagagtcttttcgctggtggcgtaatactggttttgttgagaaattaccatttgcacgtgatcgtttagttgaatgttacttttggaacactggtattattgaaccacgtcaacacgcatcagctcgtattatgatgggtaaagtaaatgcattaattacagtaattgatgacatctatgatgtttatggaacacttgaagaattagaacaattcactgatttaattcgcagatgggacataaactcaatagatcaattaccagattatatgcaattatgttttcttgcattaaacaatttcgttgatgacacttcatacgatgttatgaaagaaaagggtgttaatgttattccttacttacgtcaatcttgggtagaccttgcagacaaatatatggtagaagcacgttggttctacggtggccataaaccatcattagaagaatacttagaaaattcttggcaatctatctcaggtccatgtatgttaactcatatattctttcgtgtaacagatagctttactaaagaaactgttgattctctttacaaatatcatgatttagttagatggtcatcattcgtgcttcgtcttgctgacgacttaggtacaagcgttgaagaagtatctcgtggtgatgtgccaaaatctttacaatgctacatgagtgattataacgctagtgaggctgaagcacgtaaacacgtaaaatggttaattgcagaagtatggaaaaagatgaatgcagaacgtgtttctaaagatagtccttttggtaaagattttataggttgtgctgttgatttaggtcgtatggctcaattaatgtatcacaatggagatggtcatggtactcaacaccctattattcatcaacaaatgacacgtactttatttgaaccattcgctggtaccggtgaaaacttatactttcaaggctcaggtggcggtggaagtgattacaaagatgatgatgataaaggaaccggttaatctagactcgag

SEQ ID NO: 128 - DNA, 1713 bp, Artificial Sequence
  Description of Artificial Sequence: Synthetic polynucleotide
catatggtaccaagacgtactggtggctatcaacctacactttgggatttttcaacaattcaattatttgatagtgaatataaagaagaaaaacatcttatgcgtgctgctggtatgattgctcaagtgaacatgttacttcaagaagaagtagacagcatccaacgtcttgaattaattgatgacttacgtcgtttaggtatatcttgccactttgatcgtgaaattgtagagattttaaacagtaaatactacaccaacaatgaaattgatgaatcagatttatacagtacagcacttagattcaaacttttacgtcaatatgattttagcgttagccaagaagtttttgattgttttaaaaatgacaaaggtacagatttcaaaccatcattagttgacgatacacgtggcttattacaattatatgaagcatcatttttatcagctcagggtgaagaaactttacatttagcacgtgattttgctactaaattcttacataaaagagttttagtagataaagatatcaatttattatctagtatcgagcgtgctttagaattaccaacacactggcgtgtacaaatgcctaacgctagatcattcatcgacgcatataaaagaagaccagacatgaaccctacagtattagagttagcaaaacttgactttaacatggttcaagcacagttccaacaagaattaaaagaagccagtcgctggtggaactctacaggattagtacatgaattaccatttgtacgtgatcgtattgtggaatgttattattggactactggtgtagtagaacgtcgtgaacacggttacgaacgtattatgttaacaaaaattaacgctttagttacaacaatcgatgatgtttttgacatttatggtactttagaagaattacaactttttacaactgctattcaaagatgggacattgagtctatgaaacaacttccaccctatatgcaaatctgctacttagctttattcaacttcgtaaatgagatggcttacgatacattacgtgataaaggttttaatagtactccatatttacgcaaagcctgggtagacttagtagaaagctacttaattgaagctaaatggtattatatgggtcacaaaccaagtttagaagagtacatgaaaaactcatggatttctatcggaggtattccaattttatcacatttattctttcgtttaacagacagtatcgaagaagaagacgctgaatcaatgcataaatatcacgatatagtacgtgcctcttgtactattttacgtttagctgatgatatgggtacatcattagatgaagttgaacgtggcgatgttcctaaatctgtacaatgctatatgaatgagaaaaacgcctctgaagaagaagcacgtgaacatgttcgtagtttaattgatcagacatggaagatgatgaataaagaaatgatgacttcatcattttcaaaatacttcgtacaagtgtctgcaaatcttgctcgtatggcacaatggatatatcaacatgaaagtgatggtttcggtatgcaacactctttagttaacaaaatgcttcgtggtttactttttgaccgttatgaaggtaccggtgaaaacttatactttcaaggctcaggtggcggtggaagtgattacaaagatgatgatgataaaggaaccggttaatctagactcgag

SEQ ID NO: 129 - DNA, 1803 bp, Artificial Sequence
  Description of Artificial Sequence: Synthetic polynucleotide
catatggtaccacgtcgcatgggtgattttcattcaaacttatgggatgatgatgtaattcaatctttacccacagcttacgaagaaaaatcttatcttgaacgtgctgagaagttaattggagaagttgaaaatatgttcaacagtatgagtttagaagatggtgaacttatgagtccattaaatgatttaattcaacgcctttggattgttgattctttaggtagattaggtatccatcgtcactttaaagatgagattaaaagtgctttagattatgtttacagttactggggtgaaaacggaataggttgtggtcgtgaaagtgctgtaactgatttaaacagtacagctttaggctttcgtacacttcgtttacacggttatccagtttcatctgatgtatttaaagcatttaaaggtcaaaatggtcaattcagttgttcagaaaatatccaaacagatgaagaaattcgtggtgttcttaacttatttagagccagtttaatagccttccctggtgagaaaataatggacgaagctgaaatcttctctacaaaatacttaaaggaagcattacaaaagatcccagttagttcattatcacgtgaaatcggtgatgtacttgaatatggatggcatacatacttaccacgtttagaagcacgtaactatattcatgttttcggacaagatacagagaatacaaaaagttatgtaaaatcaaagaaacttttagaattagctaaattagaatttaacatttttcagagcttacaaaaacgtgaattagaaagccttgttcgttggtggaaagaatctggatttcctgaaatgacattctgtagacacagacacgtggaatattacacacttgcatcatgtattgcattcgaacctcagcatagtggttttcgtttaggttttgctaaaacatgtcaccttataacagttttagatgacatgtatgacactttcggcaccgtagacgaattagagttatttacagcaactatgaaacgttgggacccaagttcaattgactgccttccagaatacatgaaaggagtttacattgctgtgtatgatacagttaatgaaatggctcgtgaagctgaggaagctcaaggtcgcgatacacttacatacgctcgtgaggcctgggaggcttatatagattcttatatgcaagaagctcgctggattgctactggatacttaccttctttcgatgaatattatgaaaatggtaaggtttcatgtggtcaccgtatatctgctttacaaccaattcttactatggatattccatttccagatcacattttaaaggaagttgactttccttctaaacttaatgacttagcttgtgctatcttacgccttcgcggtgatactcgttgttacaaagcagaccgtgcacgtggtgaagaggctagttctatttcttgttatatgaaagataatccaggtgtttctgaagaagatgccttagatcatattaacgcaatgatcagtgatgttattaagggcttaaactgggaattacttaaacccgacattaacgtacctatttctgctaagaaacatgctttcgacattgctcgtgcttttcactacggttataaatatcgtgatggctattcagttgctaatgttgaaacaaaatctttagttacacgtactttacttgaatcagttccattaggtaccggtgaaaacttatactttcaaggctcaggtggcggtggaagtgattacaaagatgatgatgataaaggaaccggttaatctagactcgag

SEQ ID NO: 130 - DNA, 1788 bp, Artificial Sequence
  Description of Artificial Sequence: Synthetic polynucleotide
catatggtaccacgtagagttggtaattatcattctaatctttgggatgatgattttatacaaagtttaatttctacaccttacggtgctcctgactaccgtgaacgcgctgatcgtcttattggtgaagtaaaagatattatgtttaatttcaaatctttagaggatggtggtaatgacttattacaacgtttacttttagttgatgacgtagaacgtttaggcattgatcgtcatttcaaaaaggaaattaagactgcattagattatgtaaatagttattggaatgaaaaaggaattggttgtggtcgtgagtctgtagttacagacttaaattcaactgctttaggccttcgtaccttaagattacatggttatactgttagctctgacgttttaaatgtttttaaagataaaaatggtcaattttcatctacagctaatattcaaattgaaggtgaaattcgtggtgttttaaatctttttcgtgcctctcttgtagcttttccaggtgagaaagtgatggatgaggctgaaactttttcaacaaaatatcttcgtgaagcattacagaaaattcctgccagttcaattttatcattagaaatacgtgatgtattagaatatggatggcatactaatttaccacgtttagaagcacgtaattacatggatgttttcggtcagcacaccaagaacaaaaatgcagccgaaaaattacttgaattagcaaaattagagttcaatatctttcacagcttacaagaacgtgaattaaagcacgtttcaagatggtggaaagactctggtagtccagagatgactttctgtcgccaccgccatgtggaatattatgctttagcttcttgtattgctttcgaaccccagcacagtggtttccgtttaggttttactaaaatgagtcatttaatcacagtgttagatgatatgtatgatgtattcggtacagttgatgaattagagttatttaccgccactattaaacgttgggacccttctgctatggaatgtttaccagagtacatgaaaggtgtttacatgatggtttatcatacagttaacgaaatggctcgtgtggcagaaaaggctcaaggtagagacacattaaactatgctcgtcaagcctgggaagcatgttttgactcttatatgcaagaagcaaaatggattgcaacaggttacttacctacattcgaggaatatttagaaaatggtaaagtgagttcagcacatcgtccttgtgcattacaacctattttaactcttgatattccatttcccgatcatattcttaaagaagtggatttcccaagcaaacttaatgacttaatttgtattatcttacgtcttagaggagacacacgttgctataaagcagaccgtgcccgtggtgaagaagcatcatcaatatcttgttatatgaaagataacccaggtttaactgaagaagatgctttaaaccacattaactttatgattcgtgacgcaatccgcgaattaaactgggagttacttaaaccagataatagtgttccaattacttcaaagaaacatgcttttgatatttcacgtgtgtggcaccacggataccgttatcgtgatggttacagctttgcaaacgtggaaactaaaagtcttgtaatgcgtactgtaatagaaccagtaccattaggtaccggtgaaaacttatactttcaaggctcaggtggcggtggaagtgattacaaagatgatgatgataaaggaaccggttaatctagactcgag

SEQ ID NO: 131 - DNA, 1725 bp, Artificial Sequence
  Description of Artificial Sequence: Synthetic polynucleotide
catatggtaccacgtcgttcaggagattatcaaccaagtttatgggactttaattacattcaatctttaaacacaccttacaaagaacaacgtcattttaatcgtcaagctgagttaattatgcaagttcgtatgttattaaaggtaaaaatggaagcaattcaacaattagagttaatagatgatttacagtacttaggattatcatatttctttcaagacgaaattaaacaaatcttaagctctattcacaatgaacctcgttattttcataataatgacctttatttcactgctttaggttttagaattttacgtcaacatggttttaatgtttcagaagacgtatttgactgctttaaaatcgaaaaatgttctgactttaatgctaacttagctcaggacacaaagggtatgttacaattatatgaagctagtttcttattaagagaaggagaagatacacttgaattagctcgtcgttttagtacacgttctttacgtgaaaaatttgatgaaggtggtgacgagatagatgaagatttaagtagttggattcgtcattctttagatttaccattacactggcgtgttcaaggtttagaagctcgttggtttttagatgcctatgctcgtcgtccagatatgaaccctcttattttcaaattagctaaattaaattttaacattgttcaagctacataccaagaagaattaaaagacatctctcgttggtggaacagtagttgtttagcagagaaattacccttcgttcgcgatcgtattgtagaatgtttcttctgggctattgctgctttcgaaccacaccaatactcatatcaacgtaaaatggccgctgtaattattacatttattactattattgatgatgtttacgatgtatatggtactattgaagaattagagttattaacagatatgattcgtagatgggataataagagtattagtcaacttccttactatatgcaagtttgttatttagctctttataacttcgtaagtgaacgcgcatacgacatcttaaaagatcaacactttaacagtattccataccttcaaagaagttgggtttcattagttgagggatacttaaaagaagcatattggtactataacggttacaaaccaagtcttgaagaatatcttaataatgcaaaaattagtattagtgcacccaccattatttcacaattatactttactttagcaaatagtatcgacgaaactgccattgaaagtttataccaatatcacaacattttatacttatcaggtactatcttacgtttagctgatgatttaggaacttcacaacatgaattagaacgtggtgatgttcccaaagctattcaatgttatatgaatgatacaaatgcatcagaaagagaagctgtagaacatgttaaatttcttattcgtgaagcctggaaagaaatgaatacagttactaccgcatcagattgtccttttacagacgatcttgttgccgccgcagctaatttagctcgtgctgctcaattcatttacttagatggtgatggtcatggtgtacaacatagcgaaattcatcagcaaatgggcggtcttctttttcaaccatacgttggtaccggtgaaaacttatactttcaaggctcaggtggcggtggaagtgattacaaagatgatgatgataaaggaaccggttaatctagactcgag

SEQ ID NO: 132 - DNA, 1800 bp, Artificial Sequence
  Description of Artificial Sequence: Synthetic polynucleotide
catatggtaccacgcagaattggtgattaccatagtaacatttgggatgatgattttatccagtcactttctaccccttatggtgaaccatcttaccaagaaagagctgaacgtcttattgtagaagtgaaaaagattttcaacagtatgtacttagatgacggtcgtttaatgagttcttttaatgacttaatgcaacgtttatggattgtagactcagtagaacgtttaggtattgcccgtcacttcaaaaatgaaattacatctgcccttgactatgtttttcgttattgggaagaaaacggtataggttgtggtcgtgattctattgtaactgacttaaatagcacagctttaggttttcgtacacttcgtttacacggttatacagtttctccagaggttttaaaagcatttcaagatcaaaatggtcaattcgtttgttcaccaggacaaacagaaggtgaaattcgttcagttttaaatttatatcgtgcaagtttaattgcctttccaggcgaaaaagttatggaagaagcagaaatcttctctactcgctatttaaaagaagctcttcaaaagattccagttagcgcattatcacaagaaatcaaatttgttatggaatatggatggcatacaaatttacctagattagaagcacgtaactatattgatactttagaaaaggatacatcagcttggttaaacaaaaatgcaggtaaaaagttattagaattagctaaattagaatttaacatctttaactcattacaacaaaaagaattacaatacttacttcgctggtggaaagaatctgacttacctaaattaacctttgcacgtcatagacacgttgaattttacacattagcttcttgtattgctattgatcccaaacattcagcattccgtttaggattcgctaaaatgtgtcacttagttacagttcttgacgatatttatgatactttcggtactattgatgaacttgagttatttacttctgcaattaaacgttggaatagttctgaaattgaacacttaccagaatatatgaaatgcgtgtatatggttgtttttgaaactgttaatgaattaactcgtgaagctgagaaaacacaaggacgtaacactttaaactatgttcgtaaagcatgggaagcatattttgattcttatatggaggaagcaaagtggatctcaaacggatatttaccaatgtttgaagaataccacgaaaatggtaaagtgtcatctgcataccgtgtagcaacattacaaccaattttaactttaaacgcttggttacccgactacattcttaaaggaattgatttcccaagtcgttttaacgatttagctagttcattcttacgtttacgtggcgatactcgctgttacaaagctgaccgtgatcgtggtgaagaagctagctgcatttcttgttacatgaaagataatccaggttctaccgaagaagatgcacttaatcacattaacgctatggtaaatgacatcattaaagaattaaactgggaattattacgcagtaatgataatattcctatgttagctaaaaagcacgcttttgatattactcgtgcacttcaccacttatacatttatcgcgatggtttcagtgttgctaataaagaaactaaaaagttagttatggagacattacttgaatcaatgttatttggtaccggtgaaaacttatactttcaaggctcaggtggcggtggaagtgattacaaagatgatgatgataaaggaaccggttaatctagactcgag

SEQ ID NO: 133 - DNA, 2502 bp, Artificial Sequence
  Description of Artificial Sequence: Synthetic polynucleotide
catatggtaccacaatctgctgaaaagaacgactctttatcaagttctacattagttaagagagaatttccacccggtttctggaaagacgacttaatcgacagtttaacttcaagtcacaaagtagctgctagcgatgaaaaacgtatcgaaaccttaatttcagaaattaagaatatgtttcgttgtatgggttatggtgagacaaatccatcagcttatgatactgcttgggtagctcgcatcccagcagttgatggatcagataatcctcactttccagagactgtggaatggatcttacaaaatcaattaaaagatggttcttggggtgaaggtttttacttccttgcttatgatcgcattttagccactttagcttgtattatcacacttacactttggcgtactggagaaacacaagtacagaaaggtatcgaatttttccgcactcaagcaggtaaaatggaagatgaagcagattcacaccgtccaagtggttttgagattgtatttcctgctatgttaaaagaggctaagattttaggcttagatttaccttatgatcttccttttcttaaacaaattattgaaaagagagaagctaagttaaaacgtattcctacagatgttttatatgctttaccaactactttactttattcattagaaggtttacaagaaatagtagactggcaaaaaatcatgaaattacaaagtaaagatggtagtttcttatcttctcctgcctcaacagcagcagtatttatgagaacaggtaacaaaaagtgtttagatttcttaaatttcgtgcttaaaaagttcggtaatcatgttccatgccactatcctttagacctttttgagcgtctttgggcagttgatactgttgaaagattaggtattgaccgtcattttaaagaagaaataaaagaggctttagactatgtgtattcacactgggacgaacgtggtattggttgggctcgtgaaaaccccgttccagatattgacgatacagcaatgggtcttcgtattttacgtcttcatggttacaatgttagcagcgatgttcttaaaacatttcgtgatgaaaatggtgagttcttttgctttttaggacaaacacaaagaggtgtgactgatatgttaaatgttaatcgttgtagccatgtatctttccctggtgaaactataatggaagaggcaaaattatgtactgaacgttacttacgcaacgcattagaaaatgtagacgcttttgataagtgggcatttaagaaaaacattcgtggtgaggtagaatatgctcttaaatatccttggcataaatcaatgccacgtttagaagcacgttcatatattgaaaattacggtccagatgatgtttggttaggtaaaactgtttatatgatgccttacatttcaaatgaaaagtacttagagttagctaaacttgattttaacaaagttcagtcaatccaccagacagaacttcaagacttacgccgttggtggaaaagttctggttttacagatttaaactttacaagagaacgtgttactgaaatttacttttcacctgcatcttttatcttcgaaccagaatttagtaaatgtcgtgaggtttatacaaaaacttctaattttactgtaattttagacgatttatatgacgctcatggctctttagatgacttaaaactttttacagagagtgttaaacgttgggatttatctttagttgaccaaatgccccagcagatgaaaatctgttttgtaggtttctataatacattcaacgatattgctaaagaaggtagagaacgtcaaggtcgtgatgttttaggttatattcaaaacgtatggaaagtacaacttgaagcatatactaaagaagcagaatggtcagaagcaaaatatgttcctagttttaacgaatacattgaaaatgcttcagtttcaattgccttaggtacagtagtacttatcagtgctttatttaccggagaagttttaacagatgaagttttatctaaaattgaccgtgaaagtagattcttacagttaatgggcttaactggacgtttagtaaatgatactaaaacatatcaagctgagcgtggtcaaggtgaagttgctagtgcaattcaatgttatatgaaagaccaccctaaaattagtgaagaagaagcattacaacatgtatattctgtaatggaaaatgcattagaagaattaaatcgtgagttcgttaacaacaaaattccagacatctataaacgtcttgttttcgaaactgcacgtataatgcaattattttacatgcaaggtgatggtttaacattaagtcacgatatggaaattaaagagcacgtaaagaattgtttattccagccagtagctggtaccggtgaaaacttatactttcaaggctcaggtggcggtggaagtgattacaaagatgatgatgataaaggaaccggttaatctagactcgag

SEQ ID NO: 134 - DNA, 2511 bp, Artificial Sequence
  Description of Artificial Sequence: Synthetic polynucleotide
catatggtaccatcttcatcaacaggcacttcaaaagtagtaagcgaaacatcttcaactattgtagacgatattccacgtctttcagcaaattatcatggtgatttatggcatcacaacgtaattcagactttagaaacaccatttagagaaagttcaacttatcaagagcgtgcagatgaattagtagtgaaaatcaaagatatgttcaatgcattaggtgacggtgacatctcaccttcagcttatgatactgcatgggtagctcgtgttgctaccatttcttctgatggtagcgaaaaaccacgttttcctcaagctcttaattgggtttttaacaatcaattacaagatggatcatggggtattgaatcacattttagtttatgcgatcgtttacttaatactacaaattcagttattgctttatcagtatggaaaactggtcactcacaggttcaacaaggtgccgaatttattgctgaaaatttacgtcttttaaatgaagaagacgaattaagtcctgattttcaaattatcttcccagctttattacagaaagccaaggctttaggaatcaatttaccctatgatttaccattcatcaaatatcttagtacaacacgcgaagctcgtttaacagatgtgtcagctgctgctgacaacataccagccaatatgcttaatgcacttgaaggtttagaagaagtgattgattggaataaaatcatgcgttttcaatctaaagatggttcatttttatcttctccagctagtacagcctgtgttttaatgaatacaggtgatgaaaaatgtttcacattcttaaataacttattagataaattcggcggttgtgttccatgtatgtatagcattgatttattagaacgtttatctttagtggacaacattgaacacttaggtattggtcgtcactttaaacaagaaatcaaaggtgcattagattatgtatatcgtcattggtctgaacgcggtatcggttggggtagagactctttagttccagatttaaacaccacagctttaggtttacgcacattaagaatgcacggttataacgtgtctagtgatgtacttaacaatttcaaagacgaaaatggtcgtttctttagtagtgctggtcaaacacacgtagagttacgttctgttgtaaatctttttcgcgcctcagatttagcctttccagacgaacgtgcaatggatgatgctcgtaaattcgcagaaccatatttacgtgaagcattagctacaaaaatatcaacaaatacaaagttattcaaagaaattgaatatgttgttgaatacccttggcacatgtcaattccacgtttagaagctcgtagttatattgacagttatgatgataattatgtatggcaacgtaagactttatatcgtatgccatcattaagtaattcaaaatgtttagaacttgctaaattagatttcaatattgttcaatctttacaccaagaagaacttaaacttttaactcgttggtggaaagaatctggtatggcagacataaatttcacccgccatcgtgtagctgaagtttacttttctagtgctacatttgagccagaatatagtgctactcgtattgcattcacaaaaattggttgcttacaagtacttttcgatgatatggctgacattttcgccactttagatgagttaaaaagttttactgaaggtgttaaacgctgggacacatcattattacatgaaattcccgaatgtatgcaaacttgttttaaagtatggtttaaacttatggaagaagtaaacaacgacgtagtaaaagttcaaggaagagatatgttagcacatattcgtaaaccctgggaattatactttaattgttatgttcaagaacgtgaatggttagaagctggttatattcctacattcgaagaatatcttaaaacttatgctattagtgtaggccttggtccttgtaccttacaacctattcttttaatgggtgagttagttaaagatgatgtagtagaaaaagttcattacccttctaacatgttcgaattagtttctttaagctggcgtttaactaatgataccaaaacatatcaagcagaaaaagtacgcggtcaacaagctagtggcattgcctgttatatgaaagacaatccaggtgctactgaagaagatgctattaaacacatttgtcgtgttgttgatcgtgcattaaaagaagcaagtttcgaatatttcaagccttcaaatgacattcctatgggttgtaaatcttttatctttaacttacgtttatgtgtacaaattttctataaattcattgatggttatggtatcgcaaacgaagaaattaaggactacattcgtaaggtttatattgatccaattcaagttggtaccggtgaaaacttatactttcaaggctcaggtggcggtggaagtgattacaaagatgatgatgataaaggaaccggttaatctagactcgag

SEQ ID NO: 135 - DNA, 1206 bp, Artificial Sequence
  Description of Artificial Sequence: Synthetic polynucleotide
catatggtaccacacaagttcacaggtgttaacgctaaattccagcaaccagcattaagaaatttatctccagtggtagttgagcgcgaacgtgaggaatttgtaggattctttccacaaattgttcgtgacttaactgaagatggtattggtcatccagaagtaggtgacgctgtagctcgtcttaaagaagtattacaatacaacgcacctggtggtaaatgcaatagaggtttaacagttgttgcagcttaccgtgaactttctggaccaggtcaaaaagacgctgaaagtcttcgttgtgctttagcagtaggatggtgtattgaattattccaagcctttttcttagttgctgacgatataatggaccagtcattaactagacgtggtcaattatgttggtacaagaaagaaggtgttggtttagatgcaataaatgattcttttcttttagaaagctctgtgtatcgcgttcttaaaaagtattgccgtcaacgtccatattatgtacatttattagagctttttcttcaaacagcttaccaaacagaattaggacaaatgttagatttaatcactgctcctgtatctaaggtagatttaagccatttctcagaagaacgttacaaagctattgttaagtataaaactgctttctattcattctatttaccagttgcagcagctatgtatatggttggtatagattctaaagaagaacatgaaaacgcaaaagctattttacttgagatgggtgaatacttccaaattcaagatgattatttagattgttttggcgatcctgctttaacaggtaaagtaggtactgatattcaagataacaaatgttcatggttagttgtgcaatgcttacaaagagtaacaccagaacaacgtcaacttttagaagataattacggtcgtaaagaaccagaaaaagttgctaaagttaaagaattatatgaggctgtaggtatgagagccgcctttcaacaatacgaagaaagtagttaccgtcgtcttcaagagttaattgagaaacattctaatcgtttaccaaaagaaattttcttaggtttagctcagaaaatatacaaacgtcaaaaaggtaccggtgaaaacttatactttcaaggctcaggtggcggtggaagtgattacaaagatgatgatgataaaggaaccggttaatctagactcgag

SEQ ID NO: 136 - DNA, 1743 bp, Artificial Sequence
  Description of Artificial Sequence: Synthetic polynucleotide
catatggtaccatcattaactgaagaaaaaccaattcgcccaatcgcaaactttcctccaagcatttggggagatcaattcttaatttacgaaaaacaagtagaacaaggtgttgaacagattgttaacgaccttaagaaagaagtgcgccaacttttaaaagaggctttagatattccaatgaaacacgcaaaccttttaaaacttattgacgaaattcaacgtcttggtattccatatcactttgaacgtgaaattgatcatgcattacaatgtatctatgaaacttatggtgataattggaatggtgatcgttcttcattatggttccgtttaatgcgtaaacaaggttattatgttacatgtgacgtgtttaacaattacaaagataaaaatggtgcatttaaacaatctttagctaatgatgttgaaggtttattagaattatatgaagctacttcaatgcgtgttccaggtgaaattattcttgaagatgcattaggttttacacgttctcgtttatctattatgacaaaagacgcatttagtacaaatcctgctttatttactgaaattcagcgtgcccttaaacagcctttatggaaacgtttaccaagaattgaagctgctcaatatattccattttatcaacaacaagattctcacaataagacattacttaaattagccaaattagaatttaatcttttacaatcattacataaagaagaattaagtcatgtgtgtaaatggtggaaagcatttgatattaagaagaatgctccatgtttacgtgatagaattgtagagtgttacttttggggccttggtagtggttacgagccacaatattcacgtgctcgtgtattctttacaaaagctgttgcagttattactttaattgacgatacctatgatgcatacggaacctatgaggagcttaaaattttcactgaagctgtagaacgttggtctataacttgtttagatactttaccagaatatatgaaacccatctacaaattattcatggacacatacactgaaatggaagaatttttagcaaaagaaggtcgcacagacctttttaactgtggtaaagaatttgttaaagagtttgttcgtaacttaatggtagaagctaagtgggctaatgaaggtcacattcctactacagaagagcacgatccagtagtaataattacaggtggagcaaacttacttaccacaacttgttacttaggtatgtctgacatttttacaaaagaatcagtagagtgggcagtatctgcaccaccattattccgttattctggcatacttggtcgtcgtcttaatgatttaatgactcataaagctgaacaagagcgtaaacactcatcaagtagtttagaaagctatatgaaggaatataacgttaacgaagagtatgctcaaacacttatttacaaagaggttgaagacgtttggaaggacattaaccgtgaatacttaacaactaaaaacattccacgtcctcttttaatggctgtaatatacttatgtcaattcttagaagtacaatacgctggaaaagataactttacacgtatgggtgatgaatataaacacttaataaagagtttattagtttatcctatgtcaataggtaccggtgaaaacttatactttcaaggctcaggtggcggtggaagtgattacaaagatgatgatgataaaggaaccggttaatctagactcgag

SEQ ID NO: 137 - DNA, 2556 bp, Artificial Sequence
  Description of Artificial Sequence: Synthetic polynucleotide
catatggtaccagcaggtgtatcagctgtgtcaaaagtttcttcattagtatgtgacttaagtagtactagcggcttaattcgtagaactgcaaatcctcaccctaatgtatggggttatgacttagttcattctttaaaatctccatatattgatagtagctatcgtgaacgtgctgaagtgcttgtaagtgaaataaaagctatgttaaatccagcaattactggagatggtgaatcaatgattacaccttcagcttatgacactgcttgggttgcacgtgtaccagcaattgatggtagcgcacgtccacaatttccacaaacagtagattggattttaaagaatcaattaaaagatggttcttggggtattcaatcacactttttactttcagaccgtttattagctactcttagctgtgttttagttttacttaaatggaatgttggtgatttacaggttgagcaaggtattgagtttattaagtcaaaccttgaattagtaaaagatgaaactgatcaagattctttagtgactgattttgagattattttccctagcttacttcgtgaggcccaaagtttacgtttaggtcttccatacgatttaccttacatccacttattacaaacaaaacgtcaggaacgtttagcaaaattaagccgtgaagaaatatatgcagttccaagtccacttttatattctttagagggtattcaagatattgttgagtgggaacgtattatggaagtacaatctcaggatggatcatttttaagttctccagcatcaaccgcatgtgtttttatgcatacaggtgacgctaagtgtttagaatttcttaacagtgtaatgattaagtttggtaattttgtaccatgcctttatcctgtagatttattagaacgtttacttatagtagataatatagttcgtcttggtatttaccgtcacttcgaaaaagaaattaaagaagcattagattatgtatatcgccattggaatgaacgtggtattggttggggtcgtttaaatccaattgctgacttagaaacaactgctttaggttttcgtttattacgtttacaccgttataatgtatctccagcaatctttgataatttcaaagatgccaatggcaaattcatttgtagcactggtcagtttaataaggatgtggcttcaatgttaaacttataccgtgcatcacaattagcattcccaggcgaaaacattttagatgaagctaaatcttttgccaccaaatacttacgtgaagcccttgaaaaatctgaaacttcatcagcttggaacaataaacagaatttaagtcaagaaatcaagtatgcattaaaaacttcatggcacgcttctgtaccacgtgttgaagcaaaacgttattgtcaagtttatcgtcctgattacgctcgtattgctaagtgtgtatacaaattaccatacgttaacaacgaaaaattcttagaattaggtaaattagattttaacatcattcaatcaattcatcaagaagaaatgaaaaatgtgacaagttggtttcgtgattctggcttaccattatttactttcgctcgcgaacgtcctttagaattttacttcttagttgctgctggtacttatgaacctcaatatgctaaatgtcgtttcttattcacaaaagtagcttgtcttcaaacagtattagacgatatgtacgatacttacggtactttagacgaattaaaactttttaccgaggctgtgcgtcgttgggatttatcttttacagaaaatttacctgactatatgaaattatgttatcaaatctattatgacatcgttcatgaagtggcttgggaagctgaaaaagaacaaggtagagaattagtgtcattcttccgtaaaggctgggaagactacttattaggttactatgaagaagcagaatggttagcagcagaatacgttccaacattagatgaatacattaaaaacggtattacatcaatcggccaacgtatcttattactttcaggtgtgttaattatggatggccaacttttatcacaagaagcattagaaaaagttgattaccctggtcgtcgtgttttaactgagttaaactcacttattagccgtttagctgacgacactaaaacttataaagcagaaaaagctcgtggagaattagcctcatcaattgaatgctacatgaaagatcatcctgaatgtacagaagaagaagccttagaccacatttattctattcttgaaccagccgtaaaagaattaactcgtgaatttcttaaaccagacgacgttccatttgcttgtaaaaagatgttattcgaagaaactcgtgttacaatggtgatctttaaagatggtgatggttttggtgtatctaagttagaagttaaagatcacatcaaagaatgcttaattgaaccattaccattaggtaccggtgaaaacttatactttcaaggctcaggtggcggtggaagtgattacaaagatgatgatgataaaggaaccggttaatctagactcgag

SEQ ID NO: 138 - DNA, 948 bp, Artificial Sequence
  Description of Artificial Sequence: Synthetic polynucleotide
catatggtaccaactatgatgaatatgaattttaagtactgtcacaagattatgaagaaacattcaaaatcattcagttatgcttttgacttattaccagaagaccaacgtaaagctgtttgggcaatttacgccgtgtgccgcaaaattgatgattctattgatgtatatggtgatattcaattcttaaatcagattaaagaagacatacaaagtattgaaaaatatccatacgaacatcatcattttcaatctgacagacgtattatgatggccttacagcatgttgctcagcataaaaacattgcatttcaatcattctacaatttaattgacacagtatataaagatcaacactttacaatgtttgaaacagatgctgaactttttggttattgttacggtgtagctggtactgtgggtgaagttttaactcctatattatctgatcacgaaacacatcaaacttatgacgttgcccgtcgtttaggagagtcattacagttaatcaatattcttagagatgtaggtgaagactttgacaacgaacgtatttacttctctaaacaacgtttaaaacaatacgaagtagatattgcagaagtgtaccaaaatggtgtaaacaatcactatattgatttatgggaatattacgctgcaattgctgaaaaggattttcaagatgttatggaccaaattaaagttttctctattgaagctcagccaattattgagttagctgcacgtatttatatcgaaattttagatgaagtacgtcaagctaactacacattacatgaacgtgtttttgtagataaacgtaaaaaggctaaactttttcacgaaaataaaggtaccggtgaaaacttatactttcaaggctcaggtggcggtggaagtgattacaaagatgatgatgataaaggaaccggttaatctagactcgag

SEQ ID NO: 139 - DNA, 1611 bp, Artificial Sequence
  Description of Artificial Sequence: Synthetic polynucleotide
catatggtaccaaaaattgctgttattggtgctggtgttaccggattagctgctgctgctcgtattgctagccaaggtcatgaagttacaatcttcgaaaaaaacaataatgtaggtggtcgtatgaatcaattaaaaaaagatggttttacattcgatatgggacctacaattgttatgatgccagatgtatataaagatgtatttactgcttgcggtaaaaactatgaagattatatagagttacgtcaacttcgttacatttatgacgtatatttcgatcacgatgatcgtattactgttccaactgatttagctgaattacaacaaatgttagaatcaattgaacctggtagtacacacggatttatgtcatttttaacagatgtgtacaagaaatatgaaatcgctcgcagatatttcttagaacgtacttaccgtaaaccatcagacttctacaatatgacctctttagtacaaggtgctaaacttaaaactttaaatcacgctgatcaacttatcgaacactacattgataacgaaaagattcaaaaacttttagcattccaaactctttatatcggcattgatccaaagcgtggtcctagtttatatagtattattcctatgattgaaatgatgttcggtgtacattttatcaaaggtggtatgtatggtatggctcaaggattagctcaacttaacaaagatttaggtgttaatattgaattaaatgctgaaattgaacaaatcattatcgatcctaaattcaaacgcgcagatgcaattaaagttaatggtgacattcgcaaatttgataagattttatgtactgctgactttccttcagttgccgaatcacttatgccagatttcgcacctatcaaaaagtaccctccacataaaattgcagatttagattattcttgttcagcttttcttatgtatattggtattgacatcgacgtaactgaccaagttcgtttacataacgtaatttttagcgacgattttcgtggaaatattgaagaaattttcgaaggtcgcttaagttacgacccatcaatctatgtttatgtaccagctgtagccgataaatctttagctcctgaaggtaaaacaggcatttatgtgttaatgcctactcctgaacttaaaacaggatcaggtattgactggtcagatgaggctttaactcaacaaattaaagaaatcatttatcgtaaattagcaacaattgaagtatttgaagacattaaatcacacattgtatcagaaacaatttttactcctaatgactttgaacaaacctatcacgctaaatttggttctgctttcggtttaatgcccaccttagcacaatctaattattacagacctcaaaatgtgtcacgtgattataaagacttatatttcgcaggtgcatcaacacatccaggtgctggagttccaattgtattaacaagtgccaagataacagtagacgaaatgattaaagatattgagcgtggtgtgggtaccggtgaaaacttatactttcaaggctcaggtggcggtggaagtgattacaaagatgatgatgataaaggaaccggttaatctagactcgag

SEQ ID NO: 140 - DNA, 993 bp, Artificial Sequence
  Description of Artificial Sequence: Synthetic polynucleotide
catatggtaccagcatttgacttcgatggttacatgcttcgtaaagctaaatctgtaaataaagctcttgaagctgcagtacaaatgaaagaaccattaaaaattcatgaaagtatgcgttattctttattagctggtggtaaacgtgtacgtccaatgttatgtattgcagcttgtgaattagttggtggtgacgaaagtactgctatgcctgctgcttgcgctgtagaaatgattcatactatgagtttaatgcatgatgatttaccatgtatggataatgacgatttacgtcgtggtaaaccaacaaaccacatggcatttggtgaaagtgtagcagtattagcaggtgatgcattattatcttttgcttttgaacatgtagcagcagcaacaaaaggtgctcctccagaacgtattgttagagttttaggtgaacttgcagtttctattggttcagaaggtttagttgctggacaagtagttgacgtttgttctgaaggtatggctgaggttggtttagatcatttagaatttattcatcaccacaaaactgctgctttattacaaggttctgtagtattaggtgcaatattaggtggtggaaaagaagaagaggtagcaaaacttcgtaaattcgctaactgcattggtttacttttccaagtagtagatgatattcttgatgtaacaaaatcatctaaagaattaggtaaaacagcaggtaaagatttagttgctgataaaactacttatccaaaattaatcggtgttgagaaaagtaaagagttcgcagaccgtttaaatcgtgaagctcaagaacaacttcttcattttcatccacatagagcagcacctttaatcgctttagcaaactatattgcttatcgtgataatggtaccggtgaaaatttatattttcaaggttcaggtggcggaggttctgattataaagatgatgatgataaaggaaccggttaatctagactcgag

SEQ ID NO: 141 - DNA, 906 bp, Artificial Sequence
  Description of Artificial Sequence: Synthetic polynucleotide
catatggtaccaagtcaaccttactgggcagcaattgaggcagatattgaacgttacttaaaaaaatcaattacaattcgtccaccagaaactgtatttggtccaatgcaccacttaacttttgctgcaccagctacagctgctagtactttatgtttagcagcatgtgaacttgtaggtggtgatcgtagtcaagctatggctgcagcagcagcaatccatcttgttcatgcagctgcttatgtacatgaacatttaccattaactgatggtagtcgtccagtaagtaaaccagctatccaacataaatatggtccaaatgtagaattacttacaggtgacggtattgtaccatttggttttgaattattagcaggttctgttgatccagcacgtacagatgatccagaccgtattttacgtgtaataattgaaataagtcgtgctggtggtccagaaggtatgattagtggtttacatcgtgaagaagagattgtagatggtaatacttctcttgattttattgaatacgtttgcaaaaaaaaatatggtgaaatgcacgcatgtggtgctgcatgcggtgcaattttaggtggtgcagctgaagaagaaattcaaaaacttcgtaacttcggattatatcaaggaactttacgtggtatgatggagatgaaaaactcacaccaacttattgacgaaaatatcattggcaaacttaaagaattagctttagaagaattaggtggatttcatggtaaaaatgctgaattaatgtctagtttagtagcagaaccatcattatatgctgctggtaccggtgaaaatttatactttcaaggttctggtggtggtggcagtgattataaagacgatgatgacaaaggaaccggttaatctagactcgag

SEQ ID NO: 142 - DNA, 1095 bp, Artificial Sequence
  Description of Artificial Sequence: Synthetic polynucleotide
catatggtaccacttttatctaacaaattaagagagatggttttagcagaagttcctaaattagcatctgctgctgaatatttctttaaacgtggtgttcagggtaaacaattccgttcaacaattttattattaatggcaacagctcttgacgttcgtgttccagaagcattaattggtgaatctactgatattgtaacatctgaattacgtgtacgtcaacgtggcattgctgaaattacagaaatgattcatgtagcatcacttcttcacgatgacgttcttgacgatgctgatactcgtcgtggtgttggtagtcttaatgttgtaatgggaaacaaaatgtcagttttagcaggtgacttcttactttctcgtgcttgtggtgctcttgcagctcttaaaaacacagaagttgtagcattattagctacagcagtagaacacttagttactggtgagacaatggaaataacttcatcaactgaacaacgttattctatggattactacatgcagaaaacttattacaaaactgcttcattaatttcaaattcatgtaaagcagttgctgtattaacaggtcaaacagctgaagttgcagtattagcttttgaatatggtcgtaatttaggtttagctttccagttaattgacgacattttagatttcacaggcacatctgctagtttaggaaaaggttctttatcagatatacgtcatggtgttattactgctcctatcttatttgcaatggaagaatttcctcaattaagagaagtagtagatcaagtagaaaaagatccaagaaatgtagacatagctttagaatatttaggtaaaagtaaaggtattcaacgtgctcgtgaattagcaatggaacacgcaaatttagctgctgcagctattggttctttacctgaaacagataacgaagatgttaaacgttcacgtcgtgctttaattgatttaacacacagagtaattacacgtaacaaaggtaccggtgagaatttatactttcaaggtagtggtggaggaggtagtgactataaagatgatgacgataaaggaaccggttaatctagactcgag

SEQ ID NO: 143 - DNA, 1107 bp, Artificial Sequence
  Description of Artificial Sequence: Synthetic polynucleotide
catatggtaccagtagtttctgaacgtttaagacattctgtaacaactggtattccagcattaaaaacagcagctgaatatttctttcgtcgtggtatcgaaggaaaacgtttaagacctacattagcattattaatgagtagtgctttatcaccagctgctccatcaccagagtatttacaagttgatacaagacctgctgcagaacaccctcatgaaatgcgtcgtcgtcaacaacgtttagctgaaattgcagaattaatccatgtagcttcattacttcacgatgatgttattgatgacgcacaaacacgtcgtggtgttttaagtttaaatacatctgttggtaataaaacagctatcttagcaggtgatttcttattagctcgtgcatctgtaacattagctagtttaagaaactctgaaattgtagaattaatgtcacaggttttagaacacttagtatctggtgaaattatgcaaatgactgctacttcagaacaacttttagatttagaacattatttagcaaaaacatattgtaaaactgcttcattaatggctaatagttctcgttctgttgcagttcttgcaggtgcagctcctgaagtttgtgatatggcatggtcatacggtcgtcatttaggtattgctttccaagtagttgacgatttattagatttaacaggttcatcttctgttttaggtaaacctgctttaaacgatatgcgttctggtttagcaacagcaccagtattattcgctgcacaagaagaacctgcattacaggctcttatattacgtcgttttaaacacgacggtgacgtaacaaaagcaatgtcattaattgaacgtacacaaggcttacgtcgtgctgaagaacttgcagcacaacacgcaaaagctgctgctgatatgattcgttgcttacctacagctcaatcagaccatgcagaaattgctcgtgaagcattaattcaaattacacatcgtgttttaacacgtaaaaaaggtaccggtgaaaacttatactttcaaggttctggtggtggtggatcagattataaagatgatgatgacaaaggaaccggttaatctagactcgag

SEQ ID NO: 144 - DNA, 1002 bp, Artificial Sequence
  Description of Artificial Sequence: Synthetic polynucleotide
catatggtaccagattttccacaacaattagaagcatgtgttaaacaagcaaatcaagcattatcacgtttcatcgcaccacttccattccaaaatactcctgttgttgaaacaatgcaatatggtgcattattaggaggtaaaagattaagaccatttcttgtatatgcaacaggtcacatgtttggagtatctactaacacattagatgctccagctgctgcagttgaatgtattcatgcatatagtttaattcatgatgatttacctgcaatggatgatgatgacttaagaagaggtttacctacatgtcatgttaaatttggtgaagctaatgctattttagctggcgatgcacttcaaactcttgcattcagtattttatcagatgctgatatgccagaagtttcagatcgtgatcgtatttctatgatatctgaattagcttctgctagtggtattgctggtatgtgcggtggccaagctcttgatttagacgcagaaggaaaacacgttcctttagatgctttagagcgtatacatcgtcacaaaacaggagctttaattagagctgctgttcgtcttggtgctttatcagctggagacaaaggtcgtcgtgctttaccagttttagacaaatacgctgaaagtattggtttagcttttcaagttcaggatgatatcttagatgttgtaggtgatactgctactttaggtaaacgtcaaggtgctgatcaacagttaggcaaatctacatacccagcacttttaggtttagaacaagctcgtaaaaaagcaagagacttaattgacgatgctcgtcaaagtcttaaacaattagcagaacaatcacttgatacaagtgctttagaagcattagcagattacattattcaacgtaataaaggtaccggtgaaaatttatattttcaaggttctggtggtggaggttcagactataaagatgacgatgataaaggaaccggttaatctagactcgag

SEQ ID NO: 145 - DNA, 1257 bp, Artificial Sequence
  Description of Artificial Sequence: Synthetic polynucleotide
catatggtaccaagtgttagttgttgttgtagaaatttaggaaaaactatcaaaaaagctattccaagtcaccacttacatttacgttctttaggtggtagtttatatagaagacgtattcaatcatcttcaatggaaacagacttaaaatctacattcttaaatgtttattcagttcttaaatcagatttattacacgacccatcatttgaatttacaaatgaaagtcgtttatgggtagatagaatgcttgattataatgttcgtggcggtaaacttaatcgtggtctttctgtagtagactctttcaaattacttaaacaaggtaatgatttaactgaacaagaagttttcttatcttgtgcattaggttggtgtattgagtggttacaggcttactttttagttcttgatgatattatggataattcagttacacgtcgtggtcaaccttgttggtttcgtgtaccacaagttggtatggtagctattaatgatggcattcttcttcgtaaccatattcatcgtattcttaaaaaacacttccgtgataaaccatattatgtagatttagttgaccttttcaatgaagtagagttacaaactgcatgtggacaaatgattgatttaatcacaacatttgaaggtgaaaaagacttagctaaatatagtttatcaattcaccgtcgtattgttcaatacaaaactgcatattactcattctatttaccagttgcatgtgctcttttaatggctggcgaaaatttagaaaaccacattgatgttaaaaatgtattagtagatatgggtatttactttcaagttcaggatgattatttagactgttttgctgatcctgaaacattaggtaaaattggcactgatattgaggactttaaatgttcttggttagttgtaaaagcattagaacgttgtagtgaagaacaaacaaaaattctttacgaaaactatggcaaacctgatccatctaatgttgctaaagtaaaagatttatacaaagaattagatttagaaggcgttttcatggaatatgaatctaaatcatacgagaaattaactggtgctatcgaaggtcaccaatctaaagcaattcaagctgttcttaaatctttcttagcaaaaatctataaacgtcaaaaaggtaccggtgaaaacttatactttcaaggtagtggtggcggtggtagtgattataaagatgatgatgataaaggaaccggttaatctagactcgag

SEQ ID NO: 146 - DNA, 1131 bp, Artificial Sequence
  Description of Artificial Sequence: Synthetic polynucleotide
catatggtaccagctgatcttaaatcaacattcttagatgtttattcagtattaaaaagtgatttattacaagatccatcttttgaatttacacacgaaagtcgtcaatggttagaacgtatgttagattataatgttcgtggaggcaaattaaacagaggtttaagtgtagtagacagttacaaacttttaaaacaaggtcaagacttaacagaaaaagaaacatttttatcttgtgctttaggttggtgtattgaatggttacaagcatacttcttagttttagacgatattatggataattctgtaactagacgtggtcaaccatgttggtttcgtaaaccaaaagtaggtatgattgctattaatgatggaatacttcttcgtaaccacattcatcgtattcttaaaaaacactttcgtgaaatgccttattatgtagaccttgtagacttatttaacgaagtagaatttcaaacagcttgtggtcaaatgattgacttaattacaacatttgatggtgaaaaagacctttcaaaatattcacttcagattcaccgtcgtattgttgagtacaaaacagcatactactctttctatttacctgtagcatgtgctttacttatggcaggtgaaaatttagaaaatcacacagatgttaaaactgtattagttgatatgggtatctatttccaagttcaagatgattatttagattgcttcgctgatccagaaacattaggtaaaattggtacagatattgaagactttaaatgtagttggttagtagtaaaagcattagaacgttgtagtgaagaacaaacaaaaattctttacgaaaattatggaaaagctgaaccttcaaatgtagctaaagttaaagcattatacaaagaattagatttagagggtgcatttatggaatatgaaaaagaatcatacgagaaacttacaaaacttattgaagcacatcaatcaaaagctattcaagcagttcttaaatctttcttagctaaaatttataaacgtcaaaaaggtaccggtgaaaacttatactttcaaggctctggaggtggtggttcagactataaagatgatgatgataaaggaaccggttaatctagactcgag

SEQ ID NO: 147 - DNA, 1185 bp, Artificial Sequence
  Description of Artificial Sequence: Synthetic polynucleotide
catatggtaccaagtggcgaacctactccaaaaaaaatgaaagcaacatacgttcacgaccgtgaaaactttacaaaagtatacgaaactcttcgtgacgaattacttaacgatgattgtcttagtccagctggttcacctcaggctcaagctgctcaagagtggtttaaagaagttaatgattataatgttcctggtggaaaacttaaccgtggtatggctgtatatgacgttttagcttcagttaaaggtccagatggtttaagtgaagacgaagtatttaaagctaacgctcttggttggtgtattgagtggttacaagcatttttcttagttgctgatgatataatggatggttcaattacacgtcgtggccaaccttgttggtacaaacaacctaaagttggtatgattgcttgtaatgattacatcttattagaatgctgtatttactcaattcttaaaagacattttagaggtcacgctgcatacgctcaacttatggaccttttccatgaaactacattccagacttcacacggtcaattattagatttaacaacagcacctatcggttctgtagacttatcaaaatatacagaagataattaccttcgtattgtaacatataaaactgcatactattctttttatttacctgtagcatgtggtatggtattagctggcattacagatccagctgcttttgatcttgcaaaaaatatttgtgttgaaatgggtcaatatttccagattcaagacgattatttagattgctatggtgaccctgaggttattggtaaaatcggtacagacatagaagacaacaaatgtagttggttagtttgcacagctcttaaaatcgcaacagaagaacaaaaagaggttataaaagctaattatggtcacaaagaggctgaatcagtagcagcaattaaagcattatacgttgaattaggtattgaacaacgttttaaagactatgaagctgcatcatacgcaaaattagaaggtacaattagtgaacaaactttattacctaaagcagtatttacttctttattagctaaaatctataaaagaaaaaaaggtaccggtgagaacttatactttcaaggtagtggaggtggtggttcagactataaagatgatgatgataaaggaaccggttaatctagactcgag

SEQ ID NO: 148 - DNA, 651 bp, Artificial Sequence
  Description of Artificial Sequence: Synthetic polynucleotide
catatggtaccacaaactgaacatgttatcttattaaacgctcaaggtgttcctacaggtacattagaaaaatatgctgcacacactgctgatactcgtttacacttagctttctcatcttggttattcaatgctaaaggtcaacttttagttacaagacgtgcattaagtaaaaaagcatggcctggtgtttggactaactcagtttgtggtcatccacaattaggtgaaagtaatgaagatgcagttatacgtcgttgcagatatgaattaggtgttgaaataactccaccagaatcaatttatccagatttccgttatcgtgcaactgatcctagtggtatcgttgaaaacgaagtatgtcctgtttttgctgcacgtacaacaagtgcattacaaattaatgatgatgaagtaatggattatcaatggtgtgacttagctgatgttttacatggtattgatgcaacaccatgggcattttcaccatggatggtaatgcaagcaacaaatcgtgaagcacgtaaaagattaagtgcttttacacagttaaaaggtaccggtgaaaacttatactttcaaggtagtggaggtggtggttctgactataaagatgacgatgataaaggaaccggttaatctagactcgag

SEQ ID NO: 149 - DNA, 984 bp, Artificial Sequence
  Description of Artificial Sequence: Synthetic polynucleotide
catatggtaccacttcgtagtttattaagaggtttaacacacattcctcgtgttaatagtgctcagcaaccttcttgcgctcacgctcgtcttcaatttaaacttcgttctatgcaattattagcagaaaaccgtacagatcacatgcgtggtgcttctacatgggcaggtggtcagtctcaagatgaattaatgcttaaagatgaatgtatcttagtagatgctgatgataacattactggtcacgcttctaaattagaatgtcacaaatttcttccacatcaaccagctggattattacaccgtgctttttctgtatttcttttcgacgatcaaggtcgtttacttttacaacaacgtgctcgtagtaaaattacatttccatctgtatgggctaatacatgttgtagtcatccattacatggtcaaacaccagatgaagtagatcaacaatcacaagtagcagacggaactgtaccaggtgcaaaagctgctgcaatcagaaaattagaacatgaattaggtattccagctcaccaattaccagcatcagcttttcgtttcttaacacgtcttcactattgtgcagctgacgttcaacctgcagcaacacaatctgcattatggggtgaacacgaaatggattacattttattcattagagctaatgttacacttgctcctaatcctgacgaagtagatgaggtacgttatgtaactcaagaagaattaagacaaatgatgcaaccagataatggtttacaatggtcaccatggttccgtattattgcagcaagatttttagaacgttggtgggctgatttagatgctgcattaaatacagataaacatgaagactggggaacagttcatcacattaacgaagctggtaccggtgaaaacttatactttcaaggatcaggaggcggtggaagtgattataaagatgatgatgataaaggaaccggttaatctagactcgag

SEQ ID NO: 150 - DNA, 1740 bp, Artificial Sequence
  Description of Artificial Sequence: Synthetic polynucleotide
catatggtaccaagaagatcaggcaattataacccaacagcatgggacttcaattatatccaatcattagacaatcaatacaaaaaagaacgttactctactcgtcacgctgaattaacagttcaagttaaaaaattattagaagaagaaatggaagctgttcaaaaacttgaacttatagaggatcttaaaaacttaggcatttcttacccatttaaagataatattcaacaaatcttaaatcaaatttacaatgaacacaaatgttgtcacaactcagaagttgaagaaaaagacctttatttcactgctttacgttttagattattacgtcaacaaggttttgaagtaagtcaagaagtatttgatcactttaaaaacgaaaaaggtacagattttaaacctaatttagcagatgatactaaaggattattacaattatatgaagcatcattcttattacgtgaagcagaagacacattagaacttgctcgtcaattctctactaaacttttacaaaaaaaagttgatgaaaacggtgacgataaaattgaagataacttattactttggattagacgtagtttagaattaccattacattggcgtgtacaaagattagaagctcgtggctttttagatgcttacgttcgtagacctgatatgaatcctattgtatttgaattagcaaaattagactttaacattactcaagcaacacaacaagaagaacttaaagatttatcaagatggtggaatagtactggcttagctgaaaaacttccttttgctcgtgatcgtgtagttgaatcatatttctgggctatgggtacttttgaaccacatcaatacggataccaacgtgaattagttgctaaaatcattgcacttgctacagttgtagacgatgtttacgatgtatatggtactttagaggaattagaactttttactgatgctattcgtcgttgggaccgtgaatctattgaccaattaccatattacatgcaattatgttttcttactgtaaacaactttgtttttgagttagctcacgacgtattaaaagataaatcattcaattgtttacctcatttacaaagatcatggttagatttagctgaagcataccttgtagaagcaaaatggtatcatagtcgttatacaccttctttagaagaatatcttaatattgctcgtgtttcagtaacatgtccaactattgtttctcaaatgtattttgcattaccaattccaatcgaaaaacctgtaattgagatcatgtacaaatatcacgatatcttatacttatcaggtatgttattacgtttaccagatgacttaggaacagcatcattcgaacttaaacgtggtgatgtacaaaaagcagttcaatgttatatgaaagaacgtaatgttcctgaaaatgaagctcgtgaacatgttaaattcttaattcgtgaggcttctaaacaaattaatacagcaatggcaacagactgtccatttacagaagattttgcagttgcagcagcaaacttaggtcgtgtagcaaattttgtatatgttgatggtgatggttttggagtacaacacagtaaaatctatgagcaaattggtacacttatgtttgaaccatatccaggtaccggtgaaaacttatactttcaaggtagtggtggtggaggttctgattacaaagacgatgatgataaaggaaccggttaatctagactcgag

SEQ ID NO: 151 - DNA, 1737 bp, Artificial Sequence
  Description of Artificial Sequence: Synthetic polynucleotide
catatggtaccaagaagaagtggaaactataaacctacaatgtgggattttcaatttattcaaagtgtaaataatctttacgctggtgataaatacatggaacgtttcgatgaagtaaaaaaagaaatgaaaaaaaacttaatgatgatggttgagggtttaatagaggaattagatgttaaattagaattaatagataatttagaaagattaggtgttagttatcatttcaaaaatgaaataatgcaaatccttaaatctgtacaccagcaaatcacttgtcgtgataattcattatactctactgcattaaaatttcgtttattacgtcaacacggattccacattagtcaagacatctttaacgattttaaagatatgaatggcaatgttaaacaaagtatctgtaacgatactaaaggtttattagaactttatgaagcatctttcttatctactgaatgtgaaacaacacttaaaaacttcactgaagcacacttaaaaaattatgtttatattaaccactcatgtggagatcaatacaataacataatgatggaattagttgttcacgctttagaattaccacgtcactggatgatgcctcgtttagagacacgttggtatatatcaatttatgaacgtatgcctaatgctaatccacttttacttgaacttgctaaattagacttcaatattgttcaagctacacaccaacaagacttaaaatcattatcacgttggtggaaaaacatgtgtttagctgaaaaattatcattttctcgtaaccgtttagtagaaaatcttttctgggcagttggaactaattttgaaccacaacacagttatttccgtcgtttaatcactaaaatcattgtttttgttggtattattgatgatatttatgatgtttacggcaaacttgatgagttagaattattcactttagctgtacaacgttgggatacaaaagcaatggaagacttaccatattacatgcaagtttgttatttagctttaattaatacaacaaatgatgttgcttatgaagttcttcgtaaacataacattaatgtattaccatacttaactaaatcttggacagacttatgtaaatcatatttacaagaagctcgttggtactacaatggttacaaaccttcattagaggaatatatggataatggttggattagtatagcagttcctatggtattagcacatgcacttttcttagttacagatccaattacaaaagaagcattagaatcattaacaaactatcctgatattattcgttgctcagctacaatattccgtttaaatgatgatcttggtacaagttcagatgaattaaaacgtggagatgtaccaaaatcaattcaatgctatatgaacgaaaaaggcgtttcagaggaagaagctcgtgaacatattcgtttcttaatcaaagaaacatggaaattcatgaacactgcacaccataaagagaaaagtttattttgtgagacatttgtagaaattgcaaaaaatattgcaacaacagctcattgtatgtacttaaaaggtgattctcacggtattcaaaacactgatgttaaaaactcaataagtaatatacttttccatccaattattatcggtaccggtgaaaacctttactttcaaggttcaggtggtggcggttcagactataaagatgacgatgataaaggaaccggttaatctagactcgag

SEQ ID NO: 152 - DNA, 1752 bp, Artificial Sequence
  Description of Artificial Sequence: Synthetic polynucleotide
catatggtaccaagacgtagtggaaattatgagccatctgcatgggacttcaattacttacaatctcttaataattatcaccataaagaagaacgttacttacgtcgtcaagctgatttaattgaaaaagtaaaaatgattcttaaagaagagaaaatggaagcattacagcaattagaacttatagacgatcttcgtaatttaggtctttcatattgttttgatgatcaaattaatcatattcttacaacaatttacaaccaacattcttgtttccattatcacgaagctgcaacaagtgaagaagctaacttatatttcacagctttaggtttccgtttacttcgtgaacacggattcaaagtatcacaagaagtatttgaccgtttcaaaaatgaaaaaggtacagattttcgtccagatttagtagatgatactcaaggtttattacaactttatgaagcatctttccttcttcgtgaaggtgaagacactttagaatttgcacgtcaatttgctactaaatttcttcaaaaaaaagttgaggagaaaatgatagaagaggaaaatcttttatcttggactttacattcacttgaattaccattacattggcgtatacaacgtttagaagctaaatggtttttagatgcttatgctagtcgtcctgatatgaatccaataatctttgaattagcaaaattagaatttaacattgctcaggcacttcaacaagaagaacttaaagatttatcaagatggtggaacgatactggtattgctgaaaaattacctttcgctcgtgatagaatcgttgaatctcattattgggcaattggtactttagaaccttatcaataccgttatcagcgttcattaattgcaaaaatcattgctttaactacagttgttgatgatgtatatgatgtttacggtacattagacgaattacagttatttactgatgcaattcgtcgttgggacattgaaagtataaatcaattaccttcttatatgcaattatgttatttagctatttataatttcgtatcagaattagcttatgatattttcagagataaaggttttaattctttaccatatttacacaaaagttggcttgacttagttgaggcttactttcaagaagcaaaatggtatcattctggctacacaccatcattagaacaatacttaaatatcgctcaaatttctgtagcaagtccagctatattaagtcaaatttactttactatggctggttcaattgataaaccagtaatcgaatcaatgtacaaatatagacacattttaaacttatctggtatattacttagattaccagatgacttaggtactgctagtgatgaattaggtcgtggtgatttagcaaaagcaatgcaatgttacatgaaagagcgtaacgtttctgaagaagaagctcgtgatcatgtacgtttcttaaatcgtgaggtttcaaaacaaatgaatcctgctcgtgctgctgatgattgtccattcactgatgattttgtagtagctgctgctaatttaggaagagttgcagatttcatgtatgttgaaggcgatggtttaggtttacaatacccagctatccaccaacacatggcagaacttttatttcacccttacgcaggtaccggtgaaaacttatactttcaaggttcaggtggtggaggttctgactataaagatgatgatgataaaggaaccggttaatctagactcgag

SEQ ID NO: 153 - DNA, 1752 bp, Artificial Sequence
  Description of Artificial Sequence: Synthetic polynucleotide
catatggtaccaagaagatcaggaaattatcaacctagtgcatgggattttaactatatccaatctcttaataacaaccattctaaagaagaacgtcacttagagcgtaaagcaaaacttattgaagaagtaaaaatgttattagagcaagaaatggctgctgtacaacaattagagcttattgaagaccttaaaaacttaggtttatcttacttattccaagatgaaatcaaaataatccttaattctatttacaatcatcataaatgttttcataataatcacgaacaatgtattcacgttaatagtgacttatactttgttgcattaggcttccgtttatttcgtcaacatggtttcaaagtttctcaagaggtttttgactgttttaaaaacgaagaaggatcagactttagtgctaacttagcagatgatactaaaggtttacttcaattatacgaggcttcatatttagttacagaagatgaagacacattagaaatggcacgtcaattttcaactaaaatcttacaaaaaaaagtagaagagaaaatgattgagaaagagaacttattaagttggactttacatagtttagaattaccacttcactggcgtattcaacgtttagaagcaaaatggttccttgatgcttatgctagtcgtccagatatgaatccaattatttttgaattagctaaattagagtttaacattgctcaagcattacaacaagaagaattaaaagatttaagtagatggtggaatgatacaggcattgctgaaaaattaccttttgctcgtgatagaatagtagagagtcattactgggcaattggtactttagaaccttatcaatatagatatcaacgttcattaattgctaaaattattgctttaacaacagttgttgatgacgtttacgacgtatatggaactttagatgaattacagttatttacagacgctattcgtcgttgggatattgaatctattaatcaattaccaagttatatgcaattatgctatttagctatttataactttgtttctgaattagcatacgatatttttcgtgacaaaggattcaattctttaccttaccttcataaatcatggttagatttagtagaagcatactttgttgaagctaaatggtttcatgatggttatactccaactcttgaagaatatttaaataactcaaaaattactattatatgtcctgctattgttagtgaaatctacttcgcattcgctaattcaattgataaaacagaagttgaatcaatctacaaatatcacgatattttatatttatcaggaatgcttgcacgtttaccagacgacttaggtacttcatcatttgaaatgaaaagaggtgatgttgctaaagctattcaatgttacatgaaagaacataatgcttcagaggaagaagctcgtgaacacattcgtttcttaatgcgtgaagcatggaaacacatgaatactgctgcagctgctgatgactgtccatttgaatctgatttagtagtaggtgctgcatcattaggtagagttgcaaactttgtatatgttgaaggtgacggttttggtgtacaacattcaaaaatacatcaacaaatggctgaattacttttttatccatatcaaggtaccggtgaaaacttatactttcaaggtagtggaggtggtggtagtgactataaagacgatgacgataaaggaaccggttaatctagactcgag

SEQ ID NO: 154 - DNA, 1722 bp, Artificial Sequence
  Description of Artificial Sequence: Synthetic polynucleotide
catatggtaccaagaagaagtgctaattatcaagcaagtatttgggatgataatttcattcaaagtcttgcatctccttatgcaggagaaaaatatgcagaaaaagcagaaaaacttaaaacagaagttaaaactatgattgatcaaacaagagatgaacttaaacaattagaacttattgataacttacaacgtttaggtatatgtcatcactttcaagaccttacaaaaaaaattttacaaaaaatttatggagaagaacgtaacggagatcaccaacattacaaagaaaaaggcttacattttacagcattacgtttccgtattttacgtcaggacggttatcatgttccacaagatgtattttcatcatttatgaataaagctggtgactttgaagaatctttaagtaaagacacaaaaggtttagttagtttatatgaggcttcttacttatcaatggaaggtgaaactattttagatatggcaaaagacttttcatctcaccatttacataaaatggttgaagatgctactgacaaacgtgtagctaatcaaattatccattctcttgaaatgccacttcacagacgtgttcaaaaacttgaagcaatttggtttattcaattctacgaatgcggctctgatgctaatccaactttagtagaattagcaaaattagatttcaacatggttcaggcaacataccaagaagaattaaaacgtttatcacgttggtatgaagaaacaggcttacaagagaaactttcattcgctcgtcaccgtcttgctgaagcattcttatggtctatgggtattattccagaaggacactttggttatggtcgtatgcacttaatgaaaattggtgcttacattacattacttgatgatatttatgatgtttatggtactttagaagaacttcaagtattaacagaaattattgaacgttgggatattaacttattagatcaattacctgaatacatgcaaatcttctttttatacatgtttaattctacaaatgaacttgcttatgaaattttacgtgatcaaggtatcaatgtaatatcaaacttaaaaggattatgggtagagttatctcagtgttactttaaagaagctacttggttccataacggttacacaccaacaactgaagaatatcttaatgttgcttgtatttctgctagtggtcctgttattttattttcaggttactttactactactaatcctattaataaacacgaattacaatctttagaacgtcacgcacattcattatctatgatattacgtttagctgatgatttaggtacatcaagtgatgaaatgaaacgtggagatgtaccaaaagctattcaatgttttatgaatgacactggttgttgtgaagaagaagcacgtcaacacgtaaaaagattaatagatgctgaatggaaaaaaatgaacaaagacatcttaatggagaaaccatttaaaaatttttgtccaactgctatgaatttaggtcgtatttctatgagtttttatgaacacggagatggttatggaggtcctcactctgatacaaaaaaaaaaatggtatctttatttgtacaaccaatgaatattactattggtaccggtgaaaacctttattttcaaggttctggtggtggcggttcagattataaagatgatgacgacaaaggaaccggttaatctagactcgag

SEQ ID NO: 155 - DNA, 1731 bp, Artificial Sequence
  Description of Artificial Sequence: Synthetic polynucleotide
catatggtaccaagacgttcagctaactatcaacctagtatttggaaccacgattacattgaatcacttcgtatcgaatatgttggtgaaacatgtacacgtcaaattaacgttttaaaagaacaagttcgtatgatgttacacaaagttgttaatccattagaacaattagaattaattgaaattttacaacgtttaggtttaagttaccatttcgaagaagaaataaaacgtattttagatggtgtttacaataacgatcatggtggtgatacatggaaagcagaaaacctttatgcaacagctcttaaattccgtcttttacgtcagcacggttattctgtttctcaagaagttttcaactcttttaaagatgagcgtggcagtttcaaagcatgtttatgtgaagatactaaaggtatgttatcactttatgaagcatctttctttcttattgaaggtgaaaacattttagaggaagctagagactttagtacaaaacatcttgaagaatatgtaaaacaaaataaagagaaaaacttagctactttagttaatcactcattagaatttccattacattggcgtatgcctcgtttagaagctcgttggttcatcaatatctatcgtcataatcaagatgtaaatccaatccttttagaatttgctgaacttgacttcaatattgtacaagctgctcaccaagcagatttaaaacaagtatcaacatggtggaaatcaactggtttagtagaaaatctttcattcgctcgtgatcgtcctgtagaaaacttcttttggacagttggtcttattttccaaccacaattcggttattgtcgtagaatgtttactaaagtattcgcattaattactacaattgatgacgtatatgatgtatatggtactttagatgaattagaacttttcacagacgttgttgaaagatgggatattaatgcaatggatcaattacctgattatatgaaaatttgctttttaacattacacaatagtgttaacgaaatggcattagacactatgaaagaacaacgttttcacatcattaaataccttaaaaaagcatgggttgatctttgtcgttattacttagttgaagctaaatggtatagtaataaatatagaccttctttacaagaatacattgaaaatgcatggatttcaattggtgctccaactattttagttcatgcatatttcttcgttacaaatccaattacaaaagaagcattagactgtttagaagaatatccaaacattattcgttggagtagtattattgcacgtttagctgatgatttaggtacttcaacagacgaattaaaacgtggtgacgtaccaaaagcaattcaatgttatatgaatgaaacaggtgcttcagaagaaggtgctcgtgagtacattaaatacttaatttctgctacttggaaaaaaatgaacaaagatagagcagcatcaagtccattttcacatatcttcattgaaattgctcttaatttagcacgtatggcacaatgtttatatcaacacggtgacggccacggtttaggtaaccgtgaaacaaaagatcgtatactttcattacttattcaaccaattccattaaacaaagatggtaccggtgagaacttatactttcaaggctcaggtggtggtggttctgattacaaagatgatgatgataaaggaaccggttaatctagactcgag

SEQ ID NO: 156 - DNA, 1815 bp, Artificial Sequence
  Description of Artificial Sequence: Synthetic polynucleotide
catatggtaccaagaagaattggagactatcactcaaacttatggaatgatgacttcattcaatcattaacaacaccatacggtgctccatcatatattgaacgtgctgatagattaatatctgaagtaaaagaaatgtttaatagaatgtgtatggaagatggtgagttaatgtctccattaaatgatcttattcaaagattatggactgttgatagtgttgaacgtttaggtatagatcgtcacttcaaaaatgaaataaaagctagtttagattatgtatactcatactggaacgaaaaaggtatcggttgtggtcgtcaatcagtagttacagatttaaactctactgctcttggattaagaattttacgtcaacatggttacacagtttcaagtgaagttttaaaagtttttgaagaagaaaacggtcaatttgcttgttcaccttcacagactgagggcgaaattcgttcattcttaaacttatatcgtgcttcattaattgcttttcctggtgaaaaagtaatggaagaagctcaaatcttttctagtcgttacttaaaagaagcagttcagaaaattccagtttcaggtttatctcgtgaaataggcgatgttttagaatatggttggcacacaaacttacctcgttgggaagctcgtaactatatggacgtattcggtcaagacacaaatacatcattcaacaaaaacaaaatgcaatatatgaatacagagaaaattcttcaattagtaaaattagagtttaatatctttcattcattacaacaacgtgaattacaatgtttattacgttggtggaaagaaagtggtcttccacaattaacatttgcacgtcaccgtcacgttgaattttacactttagcttcttgtattgcatgtgaaccaaaacacagtgcatttcgtttaggttttgcaaaaatgtgtcacttagtaacagttttagatgatgtatatgacacatttggcaaaatggatgaattagaactttttactgcagctgttaaacgttgggacttatcagaaactgagcgtttacctgagtatatgaaaggtttatatgttgtagttttcgagactgttaatgaattagcacaagaagcagagaaaactcaaggacgtaatacattaaattacgttcgtaaagcatgggaagcatacttcgatagttatatgaaagaagcagaatggatctcaacaggctatttaccaacattcgaagagtattgtgaaaacggtaaagtatcaagtgcatatagagttgctgcacttcaacctattttaacattagatgtacaacttccagatgacatcttaaaaggtattgattttccatctcgtttcaatgatttagcatcttcatttcttcgtttacgtggagatactagatgttacgaggctgatcgtgctcgtggtgaagaagcaagttgtatttcttgttacatgaaagacaatccaggttcaactgaagaagatgcattaaatcacattaatgctatgataaatgatattattcgtgaattaaactgggaatttcttaaaccagactcaaatatcccaatgccagctcgtaaacatgctttcgatattacaagagctttacatcacttatatatttatcgtgacggtttttctgttgctaacaaagagactaaaaatcttgttgagaaaactttattagaatcaatgttattcggtaccggtgagaacctttattttcaaggttcaggtggtggtggttcagattataaagacgatgatgataaaggaaccggttaatctagactcgag

SEQ ID NO: 157 - DNA, 1746 bp, Artificial Sequence
  Description of Artificial Sequence: Synthetic polynucleotide
catatggtaccaagaagatcagctaattatcaacctagtcgttgggatcatcatcaccttttaagtgtagaaaacaaattcgctaaagataaacgtgtaagagaacgtgacttacttaaagaaaaagttcgtaaaatgttaaatgacgaacagaaaacttacttagatcaattagaatttattgacgatcttcaaaaattaggtgttagttatcacttcgaagcagaaatagataatatacttacaagttcatacaaaaaagatcgtacaaatatacaagaaagtgatttacacgcaactgcattagagtttcgtctttttcgtcaacacggttttaacgtttcagaagatgtatttgatgtatttatggaaaattgtggtaaattcgaccgtgatgacatttatggtttaatttcattatatgaagctagttatctttctactaaacttgacaaaaatcttcaaatctttatccgtccatttgctactcaacaattacgtgattttgtagatactcacagtaatgaagatttcggttcatgtgatatggtagaaatagttgttcaagcattagacatgccatactattggcaaatgcgtcgtttatctacacgttggtatattgatgtttatggtaaaagacaaaattacaaaaacttagtagttgttgaatttgcaaaaattgatttcaatattgttcaagctattcaccaggaagaacttaaaaatgtatcatcttggtggatggaaactggtttaggtaaacaactttattttgctcgtgatcgtattgtagagaactatttttggacaattggtcaaattcaagaacctcaatatggatatgttagacaaacaatgactaaaatcaatgctttattaacaacaattgatgatatttatgatatatacggtacattagaagaattacagttattcacagttgcatttgagaattgggacataaatcgtttagacgaattaccagaatatatgcgtttatgtttcttagttatctataacgaagtaaatagtatagcatgtgaaattcttagaacaaaaaatattaacgttattcctttcttaaaaaaatcttggactgatgtaagtaaagcatacttagttgaagctaaatggtataaatcaggccataaaccaaatttagaagagtatatgcaaaatgcacgtatttctatttcttcaccaacaatctttgttcacttttattgtgtattttcagaccaattatctattcaagttttagaaactttatcacaacaccaacaaaatgttgtaagatgtagttcttctgttttccgtttagctaatgacttagtaacttctccagatgaattagctagaggtgatgtttgtaaatcaattcaatgttatatgtcagaaactggtgcaagtgaagataaagctagatcacacgttcgtcaaatgattaatgatttatgggacgaaatgaattacgagaaaatggcacattcaagtagtatcttacatcatgattttatggagacagtaatcaatttagctagaatgtctcaatgtatgtaccaatatggtgacggacacggttctccagaaaaagctaaaattgtagatcgtgtaatgagtttacttttcaaccctattcctttagatggtaccggtgagaatttatattttcaaggctctggaggtggtggttcagattataaagatgatgacgacaaaggaaccggttaatctagactcgag

SEQ ID NO: 158 - DNA, 1728 bp, Artificial Sequence
  Description of Artificial Sequence: Synthetic polynucleotide
catatggtaccaagaagaagtgcaaactatcaaccttcattatggcaacatgaatacttattatcattaggcaacacttatgttaaagaagataatgttgaaagagtaactcttttaaaacaagaagtttctaaaatgttaaacgaaacagaaggtttacttgaacaacttgaattaattgacactttacaaagattaggtgtttcttatcattttgaacaggagattaaaaaaacattaactaatgttcatgttaaaaacgtacgtgctcataaaaatcgtattgatcgtaaccgttggggcgatttatatgcaactgcattagaatttcgtttattacgtcaacatggtttttctattgctcaagacgtttttgatggtaatattggtgttgacttagacgacaaagacattaaaggtattttaagtttatacgaagctagttacttatcaacacgtattgatacaaaacttaaagaatcaatctattacacaacaaaacgtttaagaaaattcgtagaggtaaacaaaaacgaaactaaaagttacactcttcgtcgtatggttattcacgcacttgagatgccttatcaccgtcgtgttggtcgtcttgaagctcgttggtatatcgaggtatatggagaaagacacgacatgaatcctattttattagaattagctaaattagattttaactttgttcaggctatccaccaagacgaattaaaatcattatctagttggtggtctaaaacaggattaacaaaacatttagactttgttcgtgatcgtattacagagggttacttcagtagtgtaggtgttatgtatgaaccagaatttgcatatcatcgtcaaatgcttacaaaagtatttatgcttattacaactattgatgacatctatgacatttacggtacacttgaagaattacaattattcacaactatcgttgaaaaatgggatgttaatcgtttagaagaacttcctaactatatgaaattatgcttcttatgtttagttaacgaaataaatcaaattggatattttgtattaagagataaaggttttaatgtaattccttatcttaaagagtcttgggctgacatgtgtactacatttcttaaagaagctaaatggtacaaatcaggttataaaccaaattttgaagagtatatgcaaaatggctggatttcatcatcagttccaactattcttttacacttattttgtttattaagtgaccaaactttagacattcttggttcttataatcacagtgttgttcgtagttcagcaacaattttacgtcttgcaaatgatttagctacttcttcagaagaattagcaagaggagatacaatgaaatcagttcaatgtcacatgcatgaaactggtgcttcagaagctgaatcaagagcttacattcaaggtattattggcgtagcttgggatgaccttaatatggagaaaaaatcatgtcgtttacaccagggattcttagaagcagcagcaaatttaggacgtgtagcacaatgcgtatatcaatatggagacggtcacggttgtccagataaagcaaaaacagtaaatcatgttcgtagtttattagttcacccattaccattaaacggtaccggtgaaaacctttattttcaaggtagtggtggaggtggttctgattataaagacgacgatgacaaaggaaccggttaatctagactcgag

SEQ ID NO: 159 - DNA, 1767 bp, Artificial Sequence
  Description of Artificial Sequence: Synthetic polynucleotide
catatggtaccagcttctccacctgctcatcgttcatctaaagcagcagacgaagagttaccaaaagcatcttctacattccatccatctctttggggttcatttttcttaacatatcagccacctacagctccacaacgtgcaaatatgaaagaacgtgctgaagttcttcgtgaacgtgttcgtaaagtattaaaaggttcaacaacagatcaattacctgaaacagttaacttaattcttacattacaaagacttggtttaggttattactatgaaaatgaaattgacaaattacttcatcaaatttactctaattcagattataacgtaaaagacttaaacttagtttctcaacgtttttacttacttcgtaaaaacggttatgacgtaccttctgatgttttcttatcttttaaaactgaagaaggtggtttcgcttgtgctgcagctgacacacgttcacttttaagtttatacaatgctgcttaccttcgtaaacatggtgaagaagtattagatgaagcaatttcatcaacacgtttaagattacaagacttattaggtcgtttattacctgaatcaccattcgctaaagaagtatcaagttcacttcgtacacctttattccgtcgtgtaggtattttagaagctcgtaactatattccaatctatgaaactgaagctacaagaaatgaagctgtattagagcttgctaaacttaacttcaatttacaacagcttgatttctgtgaagaattaaaacattgtagtgcatggtggaatgagatgattgctaaaagtaaattaacttttgtacgtgaccgtatagttgaagaatacttttggatgaatggtgcatgttatgatccaccatattcattaagtcgtattattcttacaaaaatcactggtttaattactattattgatgatatgttcgatactcatggtacaacagaggattgcatgaaattcgcagaagcatttggtcgttgggatgaatcagcaattcatcttcttccagaatacatgaaagatttttacattttaatgttagaaactttccagtcatttgaagatgcacttggtccagaaaaatcataccgtgtattatacttaaaacaagcaatggaacgtttagtagagttatattctaaagaaatcaaatggcgtgatgacgattatgttccaacaatgtcagaacatttacaagttagtgctgaaacaattgctacaattgctttaacttgctctgcttatgctggtatgggtgatatgtctattcgtaaagaaacatttgaatgggcattatctttccctcaattcattagaacttttggttcatttgtacgtttatcaaatgatgttgtatcaacaaaacgtgaacaaactaaagatcattcaccttcaacagttcactgttatatgaaagaacacggtacaactatggacgatgcttgtgaaaaaatcaaagaattaattgaggactcatggaaagacatgttagaacaatctttagctcttaaaggcttacctaaagtagtacctcaattagtttttgatttctctcgtactacagataacatgtatcgtgaccgtgatgctttaacatcatcagaagcattaaaagaaatgatacagttattattcgtagaacctatacctgaaggtaccggtgagaatctttattttcaaggatcaggtggtggaggctcagattacaaagatgacgacgataaaggaaccggttaatctagactcgag

SEQ ID NO: 160 - DNA, 1776 bp, Artificial Sequence
  Description of Artificial Sequence: Synthetic polynucleotide
catatggtaccagaggctttaggaaattttgattatgagagttatactaattttacaaaattaccatcatcacaatggggtgatcaattccttaaattttctatagcagattctgacttcgatgtattagaaagagaaatagaagtattaaaaccaaaagtaagagagaacatttttgtttcatcaagtactgataaagatgcaatgaaaaaaacaattttaagtattcatttcttagatagtttaggtttatcttatcacttcgaaaaagaaatagaggagagtttaaaacatgctttcgagaaaattgaagaccttattgctgatgaaaataaacttcatacaataagtacaattttccgtgtattccgtacatacggctattatatgtcttctgatgtattcaaaattttcaaaggagacgatggtaaattcaaagaaagtttaattgaagacgttaaaggtatgctttctttttatgaagctgttcattttggaacaactactgatcacattttagacgaagctcttagttttacattaaaccacttagagtcacttgcaacaggccgtcgtgcatcaccaccacatattagtaaattaatccaaaatgctttacatattcctcaacatcgtaacatccaggcattagtagctcgtgaatacattagtttttacgaacacgaagaagatcacgatgaaacattattaaaattagctaaattaaactttaaattcttacaacttcactattttcaagaattaaaaacaattacaatgtggtggactaaattagatcatacatctaatttaccaccaaattttcgtgaacgtacagttgaaacatggtttgcagctttaatgatgtatttcgaaccacaatttagtttaggtcgtattatgagtgcaaaattatatttagtaattactttcttagatgacgcatgtgatacatacggatcaatatctgaagtagagtcattagctgattgtttagaacgttgggacccagattatatggaaaatttacaaggtcacatgaaaacagcattcaaattcgttatgtatttattcaaagaatacgaagaaattttacgttcacaaggccgttcattcgtattagagaaaatgattgaggagtttaaaattatcgcacgtaaaaacttagaacttgtaaaatgggctcgtggtggtcacgttccttcttttgacgaatatatagagagtggtggtgctgagattggtacttatgctacaatcgcttgttcaattatgggtcttggtgaaattggtaaaaaagaagcatttgagtggttaatctctcgtcctaaacttgttcgtattttaggtgctaaaacacgtttaatggatgatatcgcagactttgaagaagacatggaaaaaggctatacagctaatgcacttaactattatatgaatgaacacggagtaactaaagaagaagctagtcgtgaacttgagaaaatgaatggtgatatgaacaaaattgtaaacgaagaatgtcttaaaattacaactatgccacgtcgtatcttaatgcaaagtgttaactacgctcgtagtttagatgtattatacacagctgatgatgtatataaccaccgtgaaggcaaacttaaagaatatatgagattacttttagtagatccaattttacttggtaccggtgaaaatctttattttcaaggttcaggtggtggtggttctgattataaagatgatgacgataaaggaaccggttaatctagactcgag

SEQ ID NO: 161 - DNA, 1677 bp, Artificial Sequence
  Description of Artificial Sequence: Synthetic polynucleotide
catatggtaccagagagtcaaacaacattcaaatacgaatcattagcatttacaaaacttagtcactgtcaatggacagactattttcttagtgttccaattgatgaaagtgaattagatgttattactcgtgaaattgatattcttaaaccagaagttatggagttattaagtagtcaaggagatgatgaaacaagtaaaagaaaagttcttcttattcagttattactttctttaggtttagcattccactttgaaaatgagattaaaaacatacttgaacacgcatttcgtaaaatagatgatataactggtgacgaaaaagacttatcaacaattagtattatgttccgtgttttccgtacttatggacacaatcttccaagtagtgtttttaaacgtttcacaggtgatgatggtaaatttcagcaaagtttaacagaagacgcaaaaggtattttaagtttatatgaagctgcacatttaggtactactacagattacattttagatgaagctcttaaattcacatctagtcacttaaaaagtttacttgctggtggtacatgtcgtcctcacatcttacgtttaatccgtaatacattatacttaccacaacgttggaacatggaagctgttatcgctcgtgaatacatatcattttacgagcaggaagaagatcacgataaaatgcttttacgtcttgcaaaacttaactttaaacttcttcaattacactacattaaagagcttaaaagtttcattaaatggtggatggaacttggtttaacttctaaatggccttctcaatttcgtgaacgtattgttgaagcatggttagctggattaatgatgtattttgaaccacagttctcaggtggtcgtgttattgctgcaaaattcaactatttacttacaatattagacgacgcatgtgaccactatttttctattcacgaattaacacgtttagttgcatgtgtagaacgttggtcaccagatggtattgacacattagaagatatttcacgttctgtattcaaattaatgttagatgttttcgacgatattggtaaaggtgtacgttcagaaggttctagttaccacttaaaagaaatgttagaggaattaaacactttagttcgtgctaatttagatttagttaaatgggctcgtggaatacaaacagctggtaaagaggcttatgaatgggttcgttcacgtccacgtttaatcaaatctttagcagctaaaggtagacttatggatgatattacagactttgactcagatatgagtaatggattcgcagctaatgctattaactactatatgaaacaatttgttgttacaaaagaagaagctattcttgaatgtcaacgtatgattgtagacattaacaaaactattaatgaagagttattaaaaactacttcagttccaggtcgtgtattaaaacaagctcttaactttggccgtttattagaattattatatacaaaatctgacgatatttacaattgttctgaaggcaaacttaaagaatacattgtaactcttttaattgatcctataagacttggtaccggtgaaaacttatactttcaaggttcaggcggtggtggtagtgattacaaagatgatgatgacaaaggaaccggttaatctagactcgag

SEQ ID NO: 162 - DNA, 1518 bp, Artificial Sequence
  Description of Artificial Sequence: Synthetic polynucleotide
catatggtaccagagagtcaaacaaaattcgactacgaatcattagcttttacaaaattatcacattcacaatggactgattactttttatcagtacctatagacgactctgaacttgacgcaattactcgtgaaatcgacattatcaaacctgaagttcgtaaattactttcaagtaaaggtgatgatgaaacttctaaacgtaaagtattacttatccaaagtttattatcattaggtttagcatttcattttgaaaacgaaattaaagatattttagaagatgcatttagacgtattgatgacattacaggtgatgaaaacgacttaagtactattagtattatgttccgtgtattccgtacatacggtcacaatttaccaagtagtgtttttaaacgtttcactggtgatgacggtaaatttgaacgttctttaactgaagatgctaaaggaattttatcattatatgaagctgcacatttaggaacaactactgattatattcttgatgaagcattagaatttacttcatcacacttaaaatctttacttgttggtggtatgtgtcgtccacatattttacgtcttattagaaatactttatatcttccacaacgttggaatatggaagcagtaattgcaagagaatacattagtttttatgaacaagaagaagatcacgataaaatgttacttcgtttagctaaattaaatttcaaattacttcaattacactacattaaagagttaaaaacattcattaaatggtggatggaattaggacttacatcaaaatggccttctcaatttcgtgaacgtattgttgaagcatggttagctggtcttatgatgtattttgaaccacagttttctggaggtcgtgtaatagctgctaaattcaattacttattaacaattttagatgatgcatgtgatcactatttctcaattccagaattaactcgtttagttgattgcgtagaaagatggaatcatgatggtatacatactttagaagacatctcacgtatcatctttaaacttgcattagatgtatttgatgatattggtcgtggtgttcgttctaaaggttgttcttattacttaaaagaaatgttagaagagttaaaaatcttagttcgtgcaaacttagatttagttaaatgggctcgtggtaatcaattacctagttttgaagaacacgttgaggtaggtggtattgctcttacaacatacgcaactttaatgtactcttttgttggcatgggtgaagcagtaggtaaagaagcatacgaatgggtacgttctcgtccacgtttaatcaaaagtttagcagcaaaaggtcgtcttatggacgatattactgatttcgaagtaaaaattatcaacttatttttcgaccttcttttatttgtattcggtaccggtgaaaacttatatttccagggtagtggtggaggaggttcagactacaaagatgacgatgacaaaggaaccggttaatctagactcgag

SEQ ID NO: 163 - DNA, 1740 bp, Artificial Sequence
  Description of Artificial Sequence: Synthetic polynucleotide
catatggtaccagcagctttcacagcaaatgcagttgacatgcgtccaccagttattacaattcacccacgttcaaaagatattttctctcaattttctttagatgataaattacaaaaacaatacgctcaaggaatcgaagctcttaaagaagaagctcgttctatgcttatggctgcaaaatctgctaaagtaatgatcttaattgatacacttgaacgtttaggattaggttatcactttgaaaaagaaattgaagagaaattagaagctatttacaaaaaagaggatggtgacgattatgatctttttacaactgctttaagattccgtttacttagacaacaccaacgtcgtgtaccatgttctgtttttgacaaatttatgaataaagagggtaaattcgaagaagaaccattaatttcagatgttgaaggtcttctttcattatatgacgctgcttatttacagattcacggtgaacacattttacaagaggctttaattttcactacacatcatttaactcgtattgaaccacaattagatgatcactctcctttaaaattaaaattaaaccgtgctttagaatttcctttttacagagaaatccctataatctatgcacatttttacatttcagtatatgaacgtgacgattctcgtgatgaagtattattaaaaatggctaaattatcttataatttcttacaaaacttatacaaaaaagaattaagtcaactttctcgttggtggaacaaattagaacttattcctaatttaccttatattcgtgattctgtagctggagcttatttatgggctgttgctttatatttcgaacctcaatattcagacgttcgtatggcaattgctaaacttatccaaattgcagcagctgtagatgatacttacgataattatgctactatacgtgaagctcaattattaacagaagcattagaacgtttaaatgtacacgaaattgacacattaccagattatatgaaaattgtttatcgttttgtaatgtcatggagtgaagatttcgaacgtgatgctacaattaaagaacagatgttagctacaccttatttcaaagctgaaatgaaaaaacttggtcgtgcttataatcaagaacttaaatgggttatggaacgtcaattacctagtttcgaagaatacatgaaaaactctgaaatcacttctggtgtttacattatgtttactgtaattagtccttacttaaatagtgcaacacaaaaaaacattgactggttattatcacaacctcgtttagcatcttcaactgcaattgttatgcgttgttgtaatgatttaggctctaatcaacgtgaatctaaaggaggagaagttatgacatctttagattgctatatgaaacaacacggtgctagtaaacaagaaacaatttctaaattcaaacttattatcgaagatgaatggaaaaacttaaatgaagaatgggctgcaacaacatgtcttccaaaagttatggtagaaatttttcgtaactatgcacgtattgcaggcttttgctacaaaaataacggtgatgcttatacatctccaaaaattgtacaacaatgttttgacgctttatttgtaaatccattaagaattggtaccggtgagaatttatactttcaaggctcaggtggaggtggtagtgattataaagatgatgatgataaaggaaccggttaatctagactcgag

SEQ ID NO: 164 - DNA, 1833 bp, Artificial Sequence
  Description of Artificial Sequence: Synthetic polynucleotide
catatggtaccagaatttagagttcatttacaggctgataatgaacagaaaatattccagaaccaaatgaaacctgaacctgaagcatcatatcttattaatcaacgtagatcagctaattacaaacctaatatttggaaaaatgactttttagatcaaagtttaattagtaaatacgacggtgatgaatatcgtaaattaagtgagaaattaatcgaggaagtaaaaatttatatatctgctgagacaatggacttagtagctaaattagaacttattgattctgttcgtaaattaggtttagctaatctttttgaaaaagaaattaaagaagcattagattctatcgcagctattgagtcagataatttaggtactcgtgatgacttatatggtactgctttacactttaaaattttacgtcaacatggttataaagtttctcaagatatttttggtcgtttcatggatgaaaaaggtacattagaaaatcatcacttcgctcacttaaaaggtatgttagaattatttgaagcatctaatttaggttttgaaggtgaagatattttagatgaagcaaaagcatcacttacattagctcttcgtgatagtggtcatatttgttatccagattctaacttaagtcgtgatgtagtacactcattagaattacctagtcaccgtcgtgttcaatggtttgatgttaaatggcaaattaatgcttatgaaaaagatatttgtagagttaatgcaactcttttagaattagcaaaattaaattttaacgtagtacaagcacaacttcaaaaaaacttacgtgaagcatctcgttggtgggctaacttaggtttcgctgataacttaaaattcgctcgtgatcgtttagttgaatgtttttcttgcgcagtaggcgtagcatttgaacctgaacactcttcttttcgtatctgtttaacaaaagttattaatttagttttaataattgatgacgtatacgacatatatggaagtgaagaagaattaaaacactttacaaatgctgttgatcgttgggattctcgtgaaacagaacaattaccagaatgtatgaaaatgtgctttcaagttttatacaatactacatgtgaaattgctcgtgaaattgaagaagaaaatggatggaatcaagttttacctcaattaactaaagtatgggctgatttttgtaaagcattattagtagaagctgaatggtacaataaaagtcacatcccaactttagaagaatatcttcgtaatggctgtatttcatcaagtgtttctgtattattagtacattctttctttagtattacacatgaaggtacaaaagaaatggcagatttcttacacaaaaacgaagacttattatacaacatctcattaattgtacgtttaaacaacgacttaggtacaagtgcagctgaacaagaacgtggtgattcaccatcatctattgtatgttacatgcgtgaagttaatgctagtgaagaaacagctcgtaaaaatataaaaggaatgatcgacaatgcttggaaaaaagttaatggtaaatgttttacaactaatcaagttccttttctttcttcttttatgaataacgctactaatatggctcgtgtagctcattcattatataaagacggagacggttttggcgatcaggaaaaaggtccacgtactcacatcttatctttattattccaaccattagttaacggtaccggtgaaaacttatactttcaaggttctggtggtggtggttctgactacaaagatgacgatgacaaaggaaccggttaatctagactcgag

SEQ ID NO: 165 - DNA, 1788 bp, Artificial Sequence
  Description of Artificial Sequence: Synthetic polynucleotide
catatggtaccaagtagtaatgtatcagctattcctaattcttttgaattaattcgtcgttcagctcaatttcaggcttctgtatggggtgattactttttatcttatcactctttaccacctgagaaaggtaataaagtaatggaaaaacaaactgaagaacttaaagaggaaatcaaaatggaattagtttctactactaaagatgaaccagagaaattacgtttaattgaccttattcaacgtttaggtgtatgttatcactttgaaaatgaaattaacaacattttacaacaattacaccacattactattacttctgagaaaaacggtgacgataatccttataacatgactttatgtttccgtttattacgtcaacaaggttacaatgtatctagtgaaccttttgatcgttttcgtggcaaatgggaatcttcttatgataacaatgtagaagaacttttatcattatatgaagcatctcaattaagaatgcaaggtgaagaagcattagatgaagcattctgttttgcaactgcacaattagaagctattgttcaagatcctactacagatccaatggttgcagcagaaatcagacaagcattaaaatggccaatgtacaaaaacttacctcgtttaaaagctcgtcatcatattggtttatattctgagaaaccatggcgtaatgagtcattacttaatttcgcaaaaatggacttcaataaacttcaaaatttacatcaaactgaaattgcatatatttctaaatggtgggacgattacggctttgcagaaaaactttctttcgcacgtaatcgtattgttgaaggctatttcttcgcattaggtatctttttcgaacctcaacttttaacagcacgtcttataatgacaaaagtaatcgctattggttctatgttagatgacatttatgatgtttatggtacttttgaagagttaaaacttttaacattagctttagaacgttgggataaatcagaaacaaaacaattacctaattacatgaaaatgtactacgaagcattattagatgtttttgaagaaattgagcaagaaatgtcacaaaaagaaactgaaacaacaccatactgtattcatcacatgaaagaagctactaaagaacttggacgtgtatttttagttgaagcaacttggtgtaaagaaggttatactcctaaagtagaggaatacttagacattgctttaatttcttttggtcataaattacttatggtaactgctttattaggtatgggttctcacatggctacacaacaaattgtacaatggattacatctatgccaaatatcttaaaagcatctgcagtaatatgtcgtttaatgaatgacattgtatctcataaatttgaacaagaacgtggtcatgttgcttctgctatcgaatgctacatggaacaaaaccaccttagtgaatatgaagcattaattgctcttcgtaaacaaattgatgatttatggaaagacatggtagaaaattactgtgcagtaatcacagaagacgaagtacctcgtggtgttttaatgcgtgttttaaatcttacacgtttattcaatgttatttacaaagacggtgatggatacacacaaagtcatggtagtacaaaagctcacattaaaagtcttttagttgatagtgtacctcttggtaccggtgaaaatctttactttcaaggttcaggtggaggtggttctgattataaagatgatgatgacaaaggaaccggttaatctagactcgag

SEQ ID NO: 166 - DNA, 1785 bp, Artificial Sequence
  Description of Artificial Sequence: Synthetic polynucleotide
catatggtaccaaaagacatgagtattccattattagcagctgtatcttctagtacagaagaaacagtacgtcctatcgcagattttcatccaacactttggggtaatcattttcttaaatctgctgctgacgtagaaactattgatgcagcaacacaagagcaacacgctgcattaaaacaagaagtacgtcgtatgattactacaacagcaaataaacttgcacaaaaacttcacatgattgatgctgtacaacgtttaggtgttgcttatcattttgaaaaagaaattgaagacgaattaggtaaagtaagtcacgatttagattcagatgatttatacgttgtatctttacgttttcgtttattccgtcaacaaggtgtaaaaattagttgcgatgttttcgacaaattcaaagatgacgaaggaaaattcaaagagtctcttattaacgatattagaggaatgttatcattatacgaagcagcttacttagctattagaggtgaagatattttagacgaagcaattgttttcacaactactcacttaaaaagtgttatctctattagtgatcattcacatgctaatagtaatttagctgaacaaatacgtcatagtttacaaattccacttcgtaaagctgctgcaagattagaagcacgttatttcttagatatttactctcgtgatgatttacatgatgaaacattacttaaattcgctaaacttgactttaacattcttcaagctgcacaccaaaaagaagctagtattatgactcgttggtggaacgatttaggttttcctaaaaaagttccttatgctcgtgaccgtattatagaaacttatatttggatgttattaggagtttcatacgaacctaatttagcatttggaagaatttttgcaagtaaagtagtatgtatgattacaacaattgatgatacatttgatgcttatggtacatttgaagagttaacattattcactgaagctgttacacgttgggatattggtttaattgacacattacctgaatatatgaaattcattgtaaaagctcttttagacatttaccgtgaagctgaagaagaattagctaaagaaggtagatcatacggtattccatacgctaaacaaatgatgcaagagttaatcattttatactttactgaggctaaatggttatacaaaggttacgttcctacatttgacgaatacaaaagtgtagctttacgttctattggtcttagaacattagcagtagcttcatttgtagatttaggtgactttattgctacaaaagacaattttgaatgtattcttaaaaatgcaaaaagtttaaaagctactgaaacaattggccgtttaatggatgatatagctggttacaaatttgaacagaaacgtggtcataacccatctgctgttgagtgttacaaaaatcaacacggagtatcagaagaagaagcagttaaagagcttttattagaagttgcaaacagttggaaagatattaacgaggaacttttaaatccaactacagttccattacctatgttacagcgtttattatattttgctcgttcaggtcacttcatctatgatgatggacatgatcgttatacacattctttaatgatgaaaagacaagttgcacttttattaactgaacctttagctattggtaccggtgaaaacttatactttcaaggttcaggtggtggtggatctgattataaagatgatgatgacaaaggaaccggttaatctagactcgag

SEQ ID NO: 167 - DNA, 1845 bp, Artificial Sequence
  Description of Artificial Sequence: Synthetic polynucleotide
catatggtaccagatttagctgttgagattgcaatggacttagctgttgatgacgttgagcgtcgtgtaggtgactatcatagtaacctttgggatgatgattttattcagagtttatcaacaccatacggcgcatcatcatatcgtgaacgtgctgaaagattagtaggagaagttaaagaaatgtttacttctatttctatcgaagatggtgaacttacatctgatttattacaacgtttatggatggtagataatgtagagcgtttaggcatttcacgtcatttcgagaacgaaataaaagcagctattgattatgtttattcatattggagtgacaaaggtattgtacgtggtcgtgattcagctgttcctgacttaaatagtattgctttaggttttcgtacattacgtttacacggttacacagttagtagtgatgtatttaaagttttccaagatcgtaaaggtgaatttgcttgcagtgcaattccaactgaaggagatattaaaggagttttaaacttacttcgtgcaagttatattgcattccctggtgaaaaagtaatggaaaaagctcaaacttttgcagcaacataccttaaagaagcattacagaaaattcaagtaagtagtttaagtcgtgaaatcgaatatgttcttgaatacggttggttaactaactttcctcgtttagaagcacgtaactatattgacgtattcggtgaagaaatttgtccatacttcaaaaaaccatgtattatggttgacaaacttttagaattagcaaaattagaatttaacttatttcacagtcttcaacaaacagagttaaaacatgttagtcgttggtggaaagatagtggtttctctcaattaacatttacaagacaccgtcatgttgagttttatacattagctagttgtatagcaattgaaccaaaacacagtgcttttcgtcttggttttgctaaagtttgttatttaggtatagttttagatgatatttatgacacatttggtaaaatgaaagaattagaactttttactgcagcaatcaaacgttgggacccttctactacagaatgcttacctgaatacatgaaaggtgtttatatggctttttacaattgtgttaatgaattagcacttcaagcagagaaaacacaaggtcgtgatatgttaaactatgcacgtaaagcatgggaagctctttttgatgcatttttagaagaagcaaaatggatctcttctggctatttaccaacattcgaagaatacttagaaaatggtaaagtatcttttggttatcgtgctgctacattacaaccaattttaacattagatattcctttacctttacatattttacaacagattgattttccaagtcgttttaatgatttagcttcatctattttacgtttaagaggtgatatctgtggttaccaagctgaacgtagtcgtggtgaagaagcatcatcaatttcatgttatatgaaagataatccaggttctactgaagaagatgcattatctcacattaatgcaatgatctcagacaatattaacgaattaaactgggaacttttaaaaccaaattcaaatgtaccaatttcatcaaaaaaacatgcatttgacattcttcgtgctttctatcacttatacaaatatcgtgatggcttctctatcgcaaaaattgaaactaaaaatcttgtaatgcgtacagttttagaacctgtaccaatgggtaccggtgaaaacttatactttcagggttctggtggaggtggttcagactataaagatgatgatgataaaggaaccggttaatctagactcgag

SEQ ID NO: 168 - DNA, 2526 bp, Artificial Sequence
  Description of Artificial Sequence: Synthetic polynucleotide
catatggtaccaacaagtgtatcagtagaatcaggaacagtatcttgtttatcatcaaacaacttaattagacgtacagctaatccacatcctaacatttggggatatgattttgttcactcacttaaatcaccatatacacacgactcatcatatcgtgaacgtgctgagactttaatttcagaaataaaagttatgcttggaggtggtgaattaatgatgactccatcagcttatgatacagcatgggtagctcgtgttccatcaattgacggtagtgcttgtccacaatttccacaaactgttgaatggattcttaaaaaccaattaaaagatggtagttggggaactgaatctcacttcttacttagtgacagattattagctacattaagttgtgtattagcattattaaaatggaaagtagctgatgttcaagtagagcaaggtattgagtttatcaaacgtaatttacaagctattaaagacgaacgtgatcaagacagtttagtaactgatttcgagattattttcccatcacttttaaaagaggctcaatctttaaacttaggcttaccttatgatttaccatatattagattattacaaacaaaacgtcaagaacgtcttgctaacttaagtatggataaaattcacggtggtactttattatcatctttagagggcattcaagatatagttgaatgggaaacaattatggatgtacaatctcaagatggttctttcttatcatcaccagcttctacagcatgtgtattcatgcatacaggagatatgaaatgtttagatttcttaaacaacgtattaactaaatttggtagtagtgttccttgtttataccctgtagatttattagaacgtcttttaattgtagataatgtagagcgtcttggtattgaccgtcattttgaaaaagaaatcaaagaggctttagattatgtttatcgtcattggaacgatcgtggtattggttggggtcgtttatcacctatcgcagacttagaaacaacagctttaggttttcgtttacttcgtcttcatcgttacaatgtttctcctgtagtattagacaatttcaaagacgcagatggcgagttcttctgcagtacaggtcaatttaacaaagatgttgcaagtatgttatctttataccgtgcttctcaattagctttccctgaagaatcaattttagatgaagctaaatcattctcaacacaatatcttcgtgaagcattagaaaaatcagaaacattttcttcttggaatcatcgtcagagtttatcagaagaaattaaatatgctttaaaaacatcatggcacgcttcagttcctcgtgttgaagcaaaacgttattgtcaggtttaccgtcaagactatgctcatttagcaaaatcagtttataaacttcctaaagtaaataatgagaaaattcttgaattagcaaaattagattttaacattattcaatctatccatcaaaaagaaatgaaaaatgttacatcatggtttcgtgattcaggcttaccacttttcacatttgctcgtgaaagacctttagagttttactttttaatcgctggtggaacatacgaacctcaatacgcaaaatgtagattcttatttacaaaagtagcttgtttacaaactgttttagacgatatgtacgatacttacggtacaccatcagagttaaaattatttactgaggcagttcgtcgttgggatttatcattcacagaaaacttacctgattatatgaaattatgctacaaaatttactatgatattgttcatgaagttgcttgggaagtagaaaaagaacagggacgtgagcttgtttcatttttccgtaaaggttgggaagactatcttttaggttattatgaagaagctgaatggttagctgctgaatacgttcctactttagatgaatacattaaaaacggtattacatctattggtcaacgtattttacttttatcaggtgtacttattatggaaggtcaacttttatcacaagaagctcttgaaaaagtagattatccaggtcgtcgtgttttaacagaattaaacagtttaattagtcgtttagcagacgatactaaaacatacaaagcagaaaaagctcgtggtgaacttgctagtagtattgaatgttatatgaaagaccaccctggttgtcaagaagaagaagcattaaaccatatttatggcattttagaaccagctgttaaagaattaactcgtgagtttcttaaagcagatcacgtaccattcccttgcaaaaaaatgttatttgatgaaacaagagttacaatggtaattttcaaagatggtgatggtttcggtatttctaaattagaagtaaaagaccacataaaagaatgtttaattgagccattaccacttggtaccggtgaaaatctttattttcaaggtagtggtggtggcggttctgactacaaagatgacgacgataaaggaaccggttaatctagactcgag

SEQ ID NO: 169 - DNA, 1746 bp, Artificial Sequence
  Description of Artificial Sequence: Synthetic polynucleotide
catatggtaccaggttctgaagtaaatagacctttagcagactttccagcaaacatttgggaagacccattaacttctttctcaaaatctgatcttggtacagaaacatttaaagagaaacatagtactttaaaagaagctgttaaagaggcatttatgagttctaaagctaatccaatcgaaaatatcaaattcatagatgcattatgccgtttaggagtatcttatcactttgaaaaagatattgtagaacaattagataaatcatttgattgcttagattttccacaaatggtacgtcaagaaggttgcgatttatatacagttggtattatctttcaagtttttagacaatttggtttcaaattaagtgctgatgtttttgaaaaattcaaagatgaaaatggtaaattcaaaggtcacttagtaactgatgcttatggtatgttatcattatacgaagctgcacaatggggtactcacggtgaagacatcattgacgaagctcttgctttttctcgtagtcacttagaagaaatatctagtcgtagttcaccacacttagcaattcgtattaaaaacgctttaaaacatccatatcataaaggtatttcacgtattgaaacacgtcaatacattagttactatgaagaagaagaatcttgtgatccaacattattagagttcgctaaaattgactttaacttattacaaattttacaccgtgaagagttagcttgtgtaactcgttggcatcatgaaatggaatttaaaagtaaagtaacttacacacgtcatcgtattacagaagcatatttatggagtcttggaacatattttgaaccacaatacagtcaagctcgtgtaataactacaatggcattaatcttatttactgctttagacgacatgtacgatgcttacggtactatggaggagttagagttattcacagatgctatggacgaatggttaccagttgttccagatgaaattcctattccagattcaatgaaattcatttacaatgttacagttgaattttacgataaattagacgaagaattagaaaaagaaggtcgttctggttgtggtttccatcttaaaaaaagtttacaaaaaacagctaatggatatatgcaagaagcaaaatggcttaaaaaagattacattgctacatttgatgagtataaagaaaatgctattttatcttcaggttattatgcattaattgcaatgacatttgttcgtatgactgatgttgctaaattagatgcttttgaatggttaagtagtcacccaaaaattcgtgtagcaagtgaaatcatttcacgttttacagacgatatttcaagttatgaatttgaacacaaacgtgaacacgttgctacaggtattgattgttatatgcaacaattcggagttagtaaagaacgtgctgttgaagttatgggcaatatagtttctgatgcatggaaagacttaaatcaagaacttatgcgtcctcatgttttcccatttccacttcttatgcgtgttttaaatctttcaagagtaattgatgtattttatcgttaccaagatgcatatactaatccaaaattacttaaagagcacattgtttctttacttattgaaactattccaattggtaccggtgaaaacttatactttcaaggtagtggtggaggtggttctgattataaagacgacgatgacaaaggaaccggttaatctagactcgag

SEQ ID NO: 170 - DNA, 1788 bp, Artificial Sequence
  Description of Artificial Sequence: Synthetic polynucleotide
catatggtaccagaggcaattagagtatttggcttaaaacttggttcaaaattatctattcactcacaaacaaatgcttttcctgcattcaaattatctcgttttccattaacatctttccctggtaaacatgctcacttagatccattaaaagcaacaactcatccattagcttttgatggtgaagaaaataaccgtgagtttaaaaacttaggtccaagtgagtggggccatcaatttctttctgctcatgtagatttatctgaaatggatgcattagaacgtgaaattgaagctcttaaaccaaaagtacgtgatatgttaatatcaagtgaaagttcaaaaaaaaaaatcttatttctttatcttttagtatcattaggattagcttatcactttgaagatgaaattaaagaaagtttagaggatggattacagaaaattgaggaaatgatggcttcagaagatgatcttcgttttaaaggcgataatggtaaattcaaagaatgtttagcaaaagatgctaaaggtattttatctctttatgaggctgctcacatgggtacaacaactgattatattcttgatgaggctttatcatttactttaacatatatggaatcattagcagcttcaggaacatgtaaaatcaacttatcacgtcgtattagaaaagcattagatcaacctcaacacaaaaatatggaaataattgtagcaatgaaatacattcaattttatgaagaagaggaagattgcgataaaactttacttaaatttgctaaacttaactttaaattcttacaattacactatttacaagaacttaaaatcttatctaaatggtataaagaccaagactttaaatcaaaattacctccatatttccgtgaccgtcttgtagaatgtcattttgcatcattaacatgttttgagcctaaatatgctcgtgcacgtattttcttatctaaaatcttcactgttcaaattttcattgacgatacttgtgaccgttacgcatcattaggtgaagttgagtcattagctgacactatcgaacgttgggaccctgatgatcatgctatggacggattacctgattatcttaaatcagtagttaaatttgtattcaatacatttcaagaatttgaacgtaaatgtaaacgttcacttcgtattaacttacaagtagcaaaatgggttaaagctggtcacttaccatcttttgatgagtatcttgatgtagctggtttagaattagctatttcattcactttcgctggtatcttaatgggcatggaaaatgtttgtaaacctgaagcatacgaatggttaaaatctcgtgacaaacttgttcgtggtgtaatcacaaaagttcgtttacttaatgatatttttggctatgaagatgatatgcgtcgtggttatgtaacaaattcaataaactgctacaaaaaacaatatggagtaacagaggaagaagctattcgtaaattacatcaaatcgttgctgatggagagaaaatgatgaatgaagagttcttaaaacctattaatgtaccatatcaggttcctaaagtagttattttagacactttacgtgcagctaatgtttcatacgaaaaagatgacgaatttacacgtccaggcgaacaccttaaaaactgcattacatctatttacttcgatttaggtaccggtgaaaacttatactttcaaggtagtggtggcggtggtagtgattacaaagatgatgatgataaaggaaccggttaatctagactcgag

SEQ ID NO: 171 - DNA, 1143 bp, Artificial Sequence
  Description of Artificial Sequence: Synthetic polynucleotide
catatggtaccaactacaacattatcatctaaccttaactcacaattcatgcaggtttacgagactcttaaatcagaacttattcatgacccattatttgagttcgatgacgattcaagacaatgggtagaacgtatgattgattatactgtaccaggtggtaaaatggttcgtggttatagtgtagtagatagttatcaattacttaaaggtgaagaacttacagaagaagaggcatttttagcttgtgcacttggttggtgtacagaatggtttcaagcattcattcttttacatgatgatatgatggatggtagtcacacaagacgtggtcaaccatgttggtttcgtttacctgaggttggtgctgttgctattaatgatggtgttttacttcgtaatcacgttcaccgtattcttaaaaaacattttcaaggtaaagcatattatgttcatttagttgatttattcaatgaaactgaatttcaaacaattagtggacaaatgatcgacttaattacaacattagttggtgaaaaagacttatctaaatattcattaagtattcatcgtcgtatcgttcaatacaaaacagcatactactcattttacttaccagttgcttgtgctttacttatgtttggtgaggatcttgataaacatgtagaagttaaaaatgttcttgttgaaatgggtacatattttcaagttcaagatgattatttagattgttttggtgctccagaagttattggcaaaattggtactgatattgaagactttaaatgttcatggttagtagttaaagcattagaattagcaaatgaagaacagaaaaaaactttacacgaaaattatggaaaaaaagatccagcatcagttgctaaagttaaagaagtataccacacacttaatttacaagctgttttcgaagattatgaagcaacatcatacaaaaaacttattacttctattgaaaatcacccatctaaagctgttcaagctgttttaaaatctttcttaggcaaaatatacaaacgtcaaaaaggtaccggtgaaaacttatactttcaaggttctggtggcggtggaagtgattacaaagatgatgacgataaaggaaccggttaatctagactcgag

SEQ ID NO: 172 - DNA, 1818 bp, Artificial Sequence
  Description of Artificial Sequence: Synthetic polynucleotide
catatggtaccaagtcaaccttactgggctgcaattgaagcagacattgaaagatatttaaaaaaatcaattacaattcgtccaccagaaactgtatttggtcctatgcaccatttaacatttgctgctcctgctactgcagctagtacattatgccttgctgcttgtgaattagttggcggtgatcgtagtcaagctatggcagctgctgctgctatccatttagttcatgcagctgcttacgttcacgaacatcttcctttaacagatggatcacgtcctgtaagtaaacctgctattcaacataaatatggtccaaacgttgaacttttaacaggtgatggtatcgttcctttcggttttgagttattagcaggttcagtagatccagcacgtactgatgaccctgatcgtattttacgtgtaattattgaaatttctcgtgctggtggaccagaaggcatgatttctggtttacaccgtgaggaagaaatcgtagatggtaacacatcattagactttatagaatatgtatgcaaaaaaaaatacggtgaaatgcacgcatgtggtgcagcttgcggagctattttaggtggagctgctgaagaagaaattcaaaaacttcgtaactttggtctttatcaaggcacattacgtggtatgatggaaatgaaaaatagtcatcagttaattgacgaaaatatcattggaaaacttaaagaacttgctcttgaagaattaggtggattccacggtaaaaacgctgaattaatgagttctttagttgctgaacctagtttatatgcagcttcatcaaataacttaggtatcgaaggtcgttttgactttgacggttacatgcttcgtaaagcaaaatctgtaaataaagcattagaagctgctgttcaaatgaaagaaccacttaaaattcacgaatcaatgcgttattcattattagctggtggtaaacgtgttcgtccaatgttatgtattgcagcttgtgaacttgttggtggtgacgaatctacagcaatgcctgcagcatgtgctgttgaaatgattcacacaatgtctttaatgcatgatgaccttccatgtatggataacgatgacttacgtcgtggtaaacctacaaaccacatggcttttggtgagtctgtagctgttcttgctggtgatgcattacttagttttgcttttgaacatgttgctgctgcaacaaaaggcgcaccacctgaacgtatcgtacgtgtattaggtgaattagctgttagtattggttcagaaggacttgtagcaggtcaagttgtagacgtttgttctgaaggcatggctgaagtaggattagatcatcttgaatttattcaccatcataaaactgctgcattattacaaggttcagttgttttaggtgcaatattaggaggcggtaaagaagaagaagtagctaaacttcgtaaatttgctaactgtattggtttacttttccaagttgttgatgatattttagatgttactaaaagtagtaaagagttaggtaaaactgcaggtaaagacttagtagctgataaaactacatatcctaaacttataggcgttgaaaaatcaaaagaatttgctgaccgtttaaatcgtgaagcacaagaacaattattacattttcatcctcaccgtgctgctccattaatcgctttagctaactacatcgcttaccgtgataatggtaccggtgaaaacttatacttccagggtagtggtggtggcggatcagattataaagatgacgatgataaaggaaccggttaatctagactcgag

SEQ ID NO: 173 - DNA, 1386 bp, Artificial Sequence
  Description of Artificial Sequence: Synthetic polynucleotide
catatggtaccagtaacagcagcacgtgcaacaccaaaattaagtaatagaaaattacgtgttgctgtaattggaggcggtccagcaggaggtgcagctgctgaaacattagcacaaggaggtattgaaacaattcttatcgaacgtaaaatggataattgtaaaccatgtggtggtgctattccattatgtatggtaggagagttcaatttacctttagacattattgaccgtcgtgtaacaaaaatgaaaatgatctctccttcaaacattgcagttgatatcggtcgtacacttaaagaacacgaatatattggtatggttcgtcgtgaggtacttgatgcttatcttcgtgaacgtgcagaaaaatcaggtgctactgttattaacggtttattcttaaaaatggatcacccagaaaattgggattcaccatatacacttcactacacagagtatgatggaaaaacaggtgctacaggaactaaaaaaactatggaagtagatgctgttattggtgctgatggtgctaattctcgtgttgcaaaaagtattgacgcaggtgattatgattatgctattgcatttcaagaacgtattcgtatacctgatgagaaaatgacttattatgaggacttagctgagatgtatgtaggtgatgatgtatcaccagacttctacggttgggtattcccaaaatgtgatcatgtagctgttggtacaggtactgtaacacataaaggtgatatcaaaaaattccagttagctacacgtaatcgtgctaaagataaaattcttggtggcaaaataatccgtgtagaggctcatcctattccagagcatcctagaccacgtcgtttatcaaaacgtgttgcattagtaggcgacgcagcaggttacgttactaaatgttcaggagaaggaatttacttcgcagctaaatctggtcgtatgtgtgctgaagctatcgttgaaggttcacaaaatggcaaaaaaatgatagatgaaggcgatttaagaaaatacttagaaaaatgggataaaacttacttaccaacttatcgtgttttagatgtacttcaaaaagttttctatcgttctaacccagctcgtgaggcttttgttgaaatgtgtaacgatgagtatgtacagaaaatgacatttgattcttacctttataaacgtgtagctcctggtagtccattagaagatatcaaattagctgtaaatactattggttcacttgttcgtgctaacgcattacgtcgtgaaattgagaaattatcagtaggtaccggtgagaatctttactttcaaggatcaggtggtggtggttctgattataaagatgacgatgataaaggaaccggttaatctagactcgag

SEQ ID NO: 174 - DNA, 1377 bp, Artificial Sequence
  Description of Artificial Sequence: Synthetic polynucleotide
catatggtaccagtagctgttattggtggtggtccaagtggcgcttgtgcagcagaaactttagcaaaaggtggtgtagaaactttcttacttgagcgtaaattagataattgtaaaccttgtggaggtgcaattccattatgtatggttgaagaatttgatttaccaatggaaataattgaccgtcgtgttactaaaatgaaaatgatatcaccttcaaaccgtgaagttgatgttggaaaaactttatcagaaactgaatggatcggtatgtgtcgtcgtgaagtatttgacgattacttaagaaaccgtgcacagaaattaggtgctaatattgttaacggtttattcatgcgttcagaacaacaatctgcagagggtccattcacaattcactataattcttatgaagacggtagtaaaatgggaaaacctgctactttagaagttgatatgataattggtgcagatggagcaaattctcgtattgcaaaagagatagatgcaggtgaatacgactacgctatagcttttcaagaacgtattcgtattcctgatgataaaatgaaatattacgaaaaccttgctgaaatgtatgtaggtgatgacgtatctcctgatttctatggttgggtttttcctaaatatgatcacgttgctgttggtacaggtactgttgtaaacaaaacagctattaaacaatatcaacaggcaacacgtgacagatcaaaagttaaaacagaaggtggcaaaattatacgtgttgaagcacacccaattccagaacatccacgtccacgtcgttgtaaaggtcgtgttgcattagtaggcgacgcagctggttatgttacaaaatgttctggcgagggcatttactttgctgctaaatctggtagaatggctgctgaagctattgtagaaggttctgctaacggtacaaaaatgtgtggtgaggatgcaattcgtgtttatttagataaatgggatcgtaaatattggacaacatacaaagtattagacattttacaaaaagtattttatcgtagtaatccagcacgtgaagcatttgttgaattatgtgaagatagttatgtacagaaaatgacatttgattcatacttatataaaactgttgttccaggaaacccattagacgacgtaaaattacttgttcgtacagtatcttctattttacgttcaaatgctttacgttctgttaattctaaatctgtaaatgtttctttcggctctaaagcaaatgaggaacgtgttatggctgcaggtaccggtgaaaatctttattttcaaggttcaggaggtggtggttcagattataaagatgatgatgacaaaggaaccggttaatctagactcgag

SEQ ID NO: 175 - DNA, 933 bp, Artificial Sequence
  Description of Artificial Sequence: Synthetic polynucleotide
catatggtaccagcaatggcagtaccattagatgtagtaattacatatccttcttcaggtgctgctgcttatccagtacttgttatgtataacggtttccaagctaaagctccatggtatcgtggtattgtagatcatgtttctagttggggttacacagttgttcaatatacaaatggtggcttatttcctattgttgtagatcgtgttgagttaacttatttagagccattattaacttggttagaaacacaaagtgctgatgctaaatctcctttatacggtcgtgcagatgtttctcgtttaggtacaatgggtcattcacgtggtggtaaattagcagctttacaatttgctggacgtacagatgtaagtggttgtgtattatttgaccctgtagatggaagtccaatgacaccagaatctgctgattatccttcagctacaaaagcattagcagcagctggtcgttctgctggcttagtaggtgcagctattacaggttcatgtaatccagtaggtcaaaattacccaaaattctggggtgctttagctcctggttcttggcaaatggtattatcacaagctggtcacatgcaatttgctcgtactggtaatccattcttagattggtcattagaccgtttatgtggtcgtggtacaatgatgagttcagatgttattacatatagtgcagcatttactgttgcttggtttgaaggtatttttcgtcctgctcaaagtcaaatgggtatttctaatttcaaaacttgggctaatactcaagttgcagctcgtagtatcacttttgatattaaacctatgcaatctcctcagggtaccggtgaaaacctttactttcaaggtagtggtggtggaggaagtgattataaagatgatgatgacaaaggaaccggttaatctagactcgag

SEQ ID NO: 176 - DNA, 963 bp, Artificial Sequence
  Description of Artificial Sequence: Synthetic polynucleotide
catatggtaccagcaccaccaaaaccagttcgtataacttgtccaacagtagctggcacttatcctgttgttttattctttcacggtttttatcttcgtaactatttctattcagatgttttaaatcatattgctagtcatggttacatcttagttgcaccacaattatgtaaacttttacctccaggtggccaagtagaagttgatgacgctggttcagttattaactgggcttcagagaatcttaaagcacaccttccaacttctgttaatgctaatggtaaatatacatctttagttggacattcacgtggtggcaaaacagctttcgcagttgcattaggtcacgcagctacattagatccatcaattacattttcagcattaattggtattgatccagtagcaggaactaacaaatacattcgtacagatccacacatcttaacttataaacctgaatcatttgaattagatattcctgtagctgttgtaggcactggtcttggtccaaaatggaataacgtaatgcctccatgcgcacctacagatttaaaccacgaagaattttacaaagaatgtaaagctactaaagctcactttgttgctgctgattatggtcacatggacatgttagacgacgatcttccaggttttgtaggcttcatggctggttgtatgtgtaaaaatggtcaacgtaaaaaatcagaaatgcgttcttttgtaggtggtatagttgtagcattcttaaaatattctttatggggtgaaaaagctgaaataagattaattgttaaagatcctagtgtatctcctgctaaattagacccatcaccagaattagaagaagcatcaggtatttttgttggtaccggtgaaaatctttattttcaaggttcaggtggaggtggttctgattataaagatgatgatgacaaaggaaccggttaatctagactcgag

SEQ ID NO: 177 - DNA, 921 bp, Artificial Sequence
  Description of Artificial Sequence: Synthetic polynucleotide
catatggtaccagctacaccagttgaagaaggtgattatccagttgtaatgttattacatggctaccttttatataattcattttattcacaattaatgttacatgtatcatctcacggtttcatcttaattgctccacaattatactcaattgctggtcctgatactatggatgaaattaaaagtactgctgagattatggactggttatcagttggtttaaatcactttttaccagctcaagttacacctaatttatctaaatttgcattatctggtcatagtcgtggtggtaaaactgcttttgctgtagcattaaaaaaatttggttattcttcaaacttaaaaattagtactttaattggtattgatccagtagacggaacaggtaaaggtaaacaaactccacctcctgttttagcatatttacctaatagttttgacttagacaaaacaccaattttagtaattggttcaggtttaggtgaaactgcacgtaatcctttatttcctccatgtgctcctccaggtgttaaccaccgtgagtttttccgtgaatgtcaaggtccagcatggcactttgttgctaaagattatggtcatttagacatgcttgatgatgatacaaaaggtattcgtggcaaatctagttactgtttatgcaaaaatggtgaagaacgtcgtccaatgcgtcgtttcgttggtggtttagttgttagttttcttaaagcatatcttgaaggtgatgatcgtgaattagtaaaaatcaaagatggttgtcatgaagatgtacctgttgaaattcaagaatttgaagtaattatgggtaccggtgaaaatctttactttcaaggttcaggcggtggaggttcagattataaagatgatgatgacaaaggaaccggttaatctagactcgag

SEQ ID NO: 178 - DNA, 1620 bp, Artificial Sequence
  Description of Artificial Sequence: Synthetic polynucleotide
catatggtaccaagtcacaaaaaaaaaaacgtaatcttcttcgtaactgatggtatgggtcctgcttctctttcaatggctcgttcatttaatcaacacgttaatgatttaccaattgatgatattttaacattagatgaacattttattggaagttcaagaacacgttcatcagattcacttgtaactgactcagctgctggagctacagcttttgcttgtgcacttaaatcatacaatggtgctataggtgtagatccacaccatcgtccatgtggaactgttttagaagctgctaaattagcaggttatttaacaggattagtagttactacacgtattactgatgctacaccagctagtttctcaagtcacgtagattatcgttggcaagaagatttaattgcaacacaccaattaggtgaatatcctttaggacgtgttgttgatcttcttatgggtggtggtcgttctcacttttatcctcaaggtgaaaaagctagtccatacggtcaccacggtgcacgtaaagatggtcgtgatttaatcgatgaagctcaaagtaatggctggcagtatgtaggagatcgtaaaaattttgattctttacttaaatcacatggtgaaaatgttactttaccatttttaggtttatttgctgacaacgatatcccatttgaaattgatcgtgatgaaaaagaatatcctagtttaaaagaacaagtaaaagtagcattaggtgctttagaaaaagcaagtaacgaagataaagatagtaatggtttctttttaatggtagaaggttctcgtattgatcatgctggccatcaaaacgatcctgcatctcaagtacgtgaagtattagcatttgatgaggcttttcaatatgtattagaatttgcagaaaacagtgatacagaaacagtattagtaagtacatcagatcatgaaacaggtggtttagttacttcaagacaagtaacagcatcatacccacaatatgtatggtatcctcaagtattagctaacgctacacatagtggagagtttcttaaacgtaaattagttgatttcgttcatgaacacaaaggcgcatcatcaaaaatagaaaacttcataaaacacgaaattcttgaaaaagatttaggtatttatgattatacagattctgacttagaaacacttattcatttagatgataacgctaatgcaattcaagataaacttaatgatatggtaagttttagagctcaaattggttggacaacacatggtcattcagcagttgatgtaaacatatatgcttacgcaaacaaaaaagctacatggtcttatgttcttaataacttacaaggtaatcacgaaaacacagaagttggtcaattcttagagaatttcttagaattaaacttaaatgaagttactgatttaatccgtgatacaaaacatacttctgattttgacgcaacagaaatagcaagtgaggttcaacactatgatgaatattaccacgaattaacaaatggtaccggtgaaaatctttattttcaaggttctggtggaggtggcagtgattataaagatgatgatgacaaaggaaccggttaatctagactcgag

SEQ ID NO: 179 - DNA, 1206 bp, Artificial Sequence
  Description of Artificial Sequence: Synthetic polynucleotide
catatggtaccacacaagttcacaggtgttaacgctaaattccagcaaccagcattaagaaatttatctccagtggtagttgagcgcgaacgtgaggaatttgtaggattctttccacaaattgttcgtgacttaactgaagatggtattggtcatccagaagtaggtgacgctgtagctcgtcttaaagaagtattacaatacaacgcacctggtggtaaatgcaatagaggtttaacagttgttgcagcttaccgtgaactttctggaccaggtcaaaaagacgctgaaagtcttcgttgtgctttagcagtaggatggtgtattgaattattccaagcctttttcttagtttgggacgatataatggaccagtcattaactagacgtggtcaattatgttggtacaagaaagaaggtgttggtttagatgcaataaatgattcttttcttttagaaagctctgtgtatcgcgttcttaaaaagtattgccgtcaacgtccatattatgtacatttattagagctttttcttcaaacagcttaccaaacagaattaggacaaatgttagatttaatcactgctcctgtatctaaggtagatttaagccatttctcagaagaacgttacaaagctattgttaagtataaaactgctttctattcattctatttaccagttgcagcagctatgtatatggttggtatagattctaaagaagaacatgaaaacgcaaaagctattttacttgagatgggtgaatacttccaaattcaagatgattatttagattgttttggcgatcctgctttaacaggtaaagtaggtactgatattcaagataacaaatgttcatggttagttgtgcaatgcttacaaagagtaacaccagaacaacgtcaacttttagaagataattacggtcgtaaagaaccagaaaaagttgctaaagttaaagaattatatgaggctgtaggtatgagagccgcctttcaacaatacgaagaaagtagttaccgtcgtcttcaagagttaattgagaaacattctaatcgtttaccaaaagaaattttcttaggtttagctcagaaaatatacaaacgtcaaaaaggtaccggtgaaaacttatactttcaaggctcaggtggcggtggaagtgattacaaagatgatgatgataaaggaaccggttaatctagactcgag

SEQ ID NO: 180 - DNA, 977 bp, Artificial Sequence
  Description of Artificial Sequence: Synthetic polynucleotide
atggtaccagcatttgacttcgatggttacatgcttcgtaaagctaaatctgtaaataaagctttgaagctgcagtacaaatgaaagaaccattaaaaattcatgaaagtatgcgttattctttattagctggtggtaaacgtgtacgtccaatgttatgtattgcagcttgtgaattagttggtggtgacgaaagtactgctatgcctgctgcttgcgctgtagaaatgattcatactatgagtttaatgcatgatgatttaccatgtatggataatgacgatttacgtcgtggtaaaccaacaaaccacatggcatttggtgaaagtgtagcagtattagcaggtgatgcattattatcttttgcttttgaacatgtagcagcagcaacaaaaggtgctcctccagaacgtattgttagagttttaggtgaacttgcagtttctattggttcagaaggtttagttgctggacaagtagttgacgtttgttctgaaggtatggctgaggttggtttagatcatttagaatttattcatcaccacaaaactgctgctttattacaaggttctgtagtattaggtgcaatattaggtggtggaaaagaagaagaggtagcaaaacttcgtaaattcgctaactgcattggtttacttttccaagtagtagatgatattcttgatgtaacaaaatcatctaaagaattaggtaaaacagcaggtaaagatttagttgctgataaaactacttatccaaaattaatcggtgttgagaaaagtaaagagttcgcagaccgtttaaatcgtgaagctcaagaacaacttcttcattttcatccacatagagcagcacctttaatcgctttagcaaactatattgcttatcgtgataatggtaccggtgaaaatttatattttcaaggttcaggtggcggaggttctgattataaagatgatgatgataaaggaaccggttaa

SEQ ID NO: 181 - DNA, 891 bp, Artificial Sequence; identical to SEQ ID NO: 88
  Description of Artificial Sequence: Synthetic polynucleotide

SEQ ID NO: 182 - DNA, 1080 bp, Artificial Sequence; identical to SEQ ID NO: 89
  Description of Artificial Sequence: Synthetic polynucleotide

SEQ ID NO: 183 - DNA, 1092 bp, Artificial Sequence; identical to SEQ ID NO: 90
  Description of Artificial Sequence: Synthetic polynucleotide

SEQ ID NO: 184 - DNA, 1128 bp, Artificial Sequence; identical to SEQ ID NO: 118
  Description of Artificial Sequence: Synthetic polynucleotide

SEQ ID NO: 185 - DNA, 1803 bp, Artificial Sequence; identical to SEQ ID NO: 119
  Description of Artificial Sequence: Synthetic polynucleotide

SEQ ID NO: 186 - DNA, 1191 bp, Artificial Sequence; identical to SEQ ID NO: 126
  Description of Artificial Sequence: Synthetic polynucleotide

SEQ ID NO: 187 - DNA, 1191 bp, Artificial Sequence; identical to SEQ ID NO: 82
  Description of Artificial Sequence: Synthetic polynucleotide

SEQ ID NO: 188 - DNA, 987 bp, Artificial Sequence; identical to SEQ ID NO: 91
  Description of Artificial Sequence: Synthetic polynucleotide

SEQ ID NO: 189 - DNA, 1242 bp, Artificial Sequence; identical to SEQ ID NO: 92
  Description of Artificial Sequence: Synthetic polynucleotide

SEQ ID NO: 190 - DNA, 1116 bp, Artificial Sequence; identical to SEQ ID NO: 93
  Description of Artificial Sequence: Synthetic polynucleotide

SEQ ID NO: 191 - DNA, 1170 bp, Artificial Sequence; identical to SEQ ID NO: 94
  Description of Artificial Sequence: Synthetic polynucleotide

SEQ ID NO: 192 - DNA, 1371 bp, Artificial Sequence; identical to SEQ ID NO: 120
  Description of Artificial Sequence: Synthetic polynucleotide

SEQ ID NO: 193 - DNA, 1362 bp, Artificial Sequence; identical to SEQ ID NO: 121
  Description of Artificial Sequence: Synthetic polynucleotide

SEQ ID NO: 194 - DNA, 918 bp, Artificial Sequence; identical to SEQ ID NO: 122
  Description of Artificial Sequence: Synthetic polynucleotide

SEQ ID NO: 195 - DNA, 948 bp, Artificial Sequence; identical to SEQ ID NO: 123
  Description of Artificial Sequence: Synthetic polynucleotide

SEQ ID NO: 196 - DNA, 906 bp, Artificial Sequence; identical to SEQ ID NO: 124
  Description of Artificial Sequence: Synthetic polynucleotide

SEQ ID NO: 197 - DNA, 1047 bp, Artificial Sequence
  Description of Artificial Sequence: Synthetic polynucleotide
atggtaccagctgctgctgcacctgctgagacaatgaataaatctgcagctggcgctgaagtaccagaggctttcacatcagtttttcaaccaggtaaattagcagttgaagcaattcaagtagatgaaaatgcagctcctactccacctattcctgttttaatagttgctccaaaagatgctggtacatatccagttgctatgttattacacggatttttcttacataatcacttttatgaacacttattacgtcacgttgcatctcatggctttatcattgttgctccacaattttctattagtattattccatcaggagatgctgaagacatcgctgctgctgcaaaagtagcagattggttacctgacggattaccaagtgttttaccaaaaggtgttgaaccagagttatcaaaacttgctttagctggacacagtcgtggtggtcacacagctttttctttagctttaggtcacgctaaaacacaattaactttcagtgcattaattggtttagatcctgttgctggaacaggtaaatcatctcaattacaaccaaaaattcttacttatgagccaagttcatttggtatggctatgccagttttagttattggtacaggtttaggagaagaaaaaaaaaacattttctttcctccatgtgctcctaaagacgtaaaccatgcagaattttatcgtgaatgtagaccaccatgttactattttgtaactaaagattatggccatcttgatatgttagatgatgacgctccaaaatttatcacatgtgtttgtaaagacggtaatggatgtaaaggaaaaatgcgtcgttgtgtagctggcatcatggttgctttcttaaacgctgctttaggtgaaaaagacgcagatttagaagctattttacgtgatccagcagttgctcctacaacattagacccagttgaacaccgtgttgctggtaccggtgagaatttatacttccagggatctggtggtggtggcagtgattataaagatgatgatgataaaggaaccggttaa

SEQ ID NO: 198 - DNA, 1605 bp, Artificial Sequence; identical to SEQ ID NO: 125
  Description of Artificial Sequence: Synthetic polynucleotide

SEQ ID NO: 199 - DNA, 1008 bp, Artificial Sequence
  Description of Artificial Sequence: Synthetic polynucleotide
atggtaccagctttatacgacattattaactatttctacggttcaaactctaaattcaaccgtattacatggggttttaaatcaccaactttcatcaaatggagaattactgatttcattttaatcatcgttttaattgttcttttcttcgtaacttctcaagcagagccattccatcgtcaattttatcttaacgacatgactatccaacatccttttgcagaacatgaacgtgtaactaatattcaacttggtttatattcaacagtaattcctttatcagttattatcattgttgctttaattagtacatgtccacctaaatacaaattatacaacacttgggtttcaagtattggtttacttttatcagttttaatcacatcttttgttacaaacatcgttaaaaactggtttggacgtttacgtcctgacttcttagatcgttgccaaccagctaacgatacacctaaagataaattagtttctattgaggtttgtactacagacaatttagaccgtttagctgacggttttcgtacaacaccttctggtcattcttcaatctcatttgctggtttattctatttaacattatttcttttaggtcaatctcaggcaaataatggtaaaacatcttcatggcgtacaatgatcagttttataccttggttaatggcttgttatatcgctttaagtcgtacacaagactaccgtcatcatttcattgacgtatttgttggtagttgcttaggcttaattatcgcaatttggcaatacttccgtttattcccttggttcggtggtaaccaagcaaatgattcatttaacaaccgtattatgattgaagagattaaacgtaaagaggaaattaaacaagatgaaaataactaccgtcgtatttctgatatttctactaatgtaggtaccggtgaaaacctttactttcaaggttcaggtggcggcggttcagattataaagatgatgacgacaaaggaaccggttaa

SEQ ID NO: 200 - DNA, 1023 bp, Artificial Sequence
  Description of Artificial Sequence: Synthetic polynucleotide
catatggtaccagctttatacgacattattaactatttctacggttcaaactctaaattcaaccgtattacatggggttttaaatcaccaactttcatcaaatggagaattactgatttcattttaatcatcgttttaattgttcttttcttcgtaacttctcaagcagagccattccatcgtcaattttatcttaacgacatgactatccaacatccttttgcagaacatgaacgtgtaactaatattcaacttggtttatattcaacagtaattcctttatcagttattatcattgttgctttaattagtacatgtccacctaaatacaaattatacaacacttgggtttcaagtattggtttacttttatcagttttaatcacatcttttgttacaaacatcgttaaaaactggtttggacgtttacgtcctgacttcttagatcgttgccaaccagctaacgatacacctaaagataaattagtttctattgaggtttgtactacagacaatttagaccgtttagctgacggttttcgtacaacaccttctggtcattcttcaatctcatttgctggtttattctatttaacattatttcttttaggtcaatctcaggcaaataatggtaaaacatcttcatggcgtacaatgatcagttttataccttggttaatggcttgttatatcgctttaagtcgtacacaagactaccgtcatcatttcattgacgtatttgttggtagttgcttaggcttaattatcgcaatttggcaatacttccgtttattcccttggttcggtggtaaccaagcaaatgattcatttaacaaccgtattatgattgaagagattaaacgtaaagaggaaattaaacaagatgaaaataactaccgtcgtatttctgatatttctactaatgtaggtaccggtgaaaacctttactttcaaggttcaggtggcggcggttcagattataaagatgatgacgacaaaggaaccggttaatctagactcgag

SEQ ID NO: 201 - DNA, 906 bp, Artificial Sequence; identical to SEQ ID NO: 141
  Description of Artificial Sequence: Synthetic polynucleotide

SEQ ID NO: 202 - DNA, 1095 bp, Artificial Sequence; identical to SEQ ID NO: 142
  Description of Artificial Sequence: Synthetic polynucleotide

SEQ ID NO: 203 - DNA, 1107 bp, Artificial Sequence; identical to SEQ ID NO: 143
  Description of Artificial Sequence: Synthetic polynucleotide

SEQ ID NO: 204 - DNA, 1143 bp, Artificial Sequence; identical to SEQ ID NO: 171
  Description of Artificial Sequence: Synthetic polynucleotide

SEQ ID NO: 205 - DNA, 1818 bp, Artificial Sequence; identical to SEQ ID NO: 172
  Description of Artificial Sequence: Synthetic polynucleotide

SEQ ID NO: 206 - DNA, 1206 bp, Artificial Sequence; identical to SEQ ID NO: 179
  Description of Artificial Sequence: Synthetic polynucleotide

SEQ ID NO: 207 - DNA, 1206 bp, Artificial Sequence; identical to SEQ ID NO: 135
  Description of Artificial Sequence: Synthetic polynucleotide

SEQ ID NO: 208 - DNA, 1062 bp, Artificial Sequence
  Description of Artificial Sequence: Synthetic polynucleotide
catatggtaccagctgctgctgcacctgctgagacaatgaataaatctgcagctggcgctgaagtaccagaggctttcacatcagtttttcaaccaggtaaattagcagttgaagcaattcaagtagatgaaaatgcagctcctactccacctattcctgttttaatagttgctccaaaagatgctggtacatatccagttgctatgttattacacggatttttcttacataatcacttttatgaacacttattacgtcacgttgcatctcatggctttatcattgttgctccacaattttctattagtattattccatcaggagatgctgaagacatcgctgctgctgcaaaagtagcagattggttacctgacggattaccaagtgttttaccaaaaggtgttgaaccagagttatcaaaacttgctttagctggacacagtcgtggtggtcacacagctttttctttagctttaggtcacgctaaaacacaattaactttcagtgcattaattggtttagatcctgttgctggaacaggtaaatcatctcaattacaaccaaaaattcttacttatgagccaagttcatttggtatggctatgccagttttagttattggtacaggtttaggagaagaaaaaaaaaacattttctttcctccatgtgctcctaaagacgtaaaccatgcagaattttatcgtgaatgtagaccaccatgttactattttgtaactaaagattatggccatcttgatatgttagatgatgacgctccaaaatttatcacatgtgtttgtaaagacggtaatggatgtaaaggaaaaatgcgtcgttgtgtagctggcatcatggttgctttcttaaacgctgctttaggtgaaaaagacgcagatttagaagctattttacgtgatccagcagttgctcctacaacattagacccagttgaacaccgtgttgctggtaccggtgagaatttatacttccagggatctggtggtggtggcagtgattataaagatgatgatgataaaggaaccggttaatctagactcgag

SEQ ID NO: 209 - protein, 6 aa, Artificial Sequence
  Description of Artificial Sequence: Synthetic 6x His tag
HHHHHH